(19) United States
(12) Patent Application Publication
(10) Pub. No.: US 2025/0034159 A1
(43) Pub. Date: Jan. 30, 2025

(54) INHIBITORS OF EPIDERMAL GROWTH FACTOR RECEPTOR

(71) Applicant: ORIC Pharmaceuticals, Inc., South San Francisco, CA (US)

(72) Inventors: Brian R. BLANK, Daly City, CA (US); Paul Anthony GIBBONS, San Francisco, CA (US); Chudi NDUBAKU, Montréal, Québec (CA); F. Anthony ROMERO, Redwood City, CA (US); Jae Hyuk CHANG, Alameda, CA (US); Johnny D. PHAM, Vista, CA (US); Tucker Curran ROBERTS, San Francisco, CA (US); Rakesh Harsukhlal VEKARIYA, Santa Clara, CA (US); Alexandria Daria Maria JEANNERET, Montréal, Québec (CA); Hugo de Almeida SILVA, Montréal, Québec (CA); Raphael Oluwagbemiga DADA, Laval, Québec (CA); Kyle Conner McINTOSH, Laval, Québec (CA); Estelle NEIGER, Dollard des Ormeaux, Québec (CA); William BROWN, Montréal, Québec (CA); Harumi TAKASAKI, Montréal, Québec (CA); Cynthia Charlene CRIFAR, Montréal, Québec (CA); Dilan Emine POLAT, Montréal, Québec (CA)

(21) Appl. No.: 18/703,801

(22) PCT Filed: Oct. 24, 2022
(86) PCT No.: PCT/US2022/078570

§ 371 Date: Apr. 23, 2024

Related U.S. Application Data
(60) Provisional application No. 63/343,290, filed on May 18, 2022.
(60) Provisional application No. 63/322,101, filed on Mar. 21, 2022.
(60) Provisional application No. 63/271,568, filed on Oct. 25, 2021.

(51) Int. Cl. C07D 491/048; A61K 31/519; A61K 31/5377; A61P 35/00; C07D 519/00

(52) U.S. Cl. CPC C07D 491/048; A61K 31/519; A61K 31/5377; A61P 35/00; C07D 519/00

(57) ABSTRACT

Disclosed herein are compounds of Formula (I), or a pharmaceutically acceptable salt thereof, that are inhibitors of inhibitors of epidermal growth factor receptor (EGFR), including EGFR C797S mutants. Also disclosed herein are pharmaceutical compositions comprising the compounds of Formula (I), or pharmaceutically acceptable salts thereof, and one or more pharmaceutically acceptable excipients. Further disclosed herein are methods of treating cancer in a subject in need thereof, comprising administering to the subject an amount of a compound of Formula (I), or a pharmaceutically acceptable salt thereof. Formula (I)

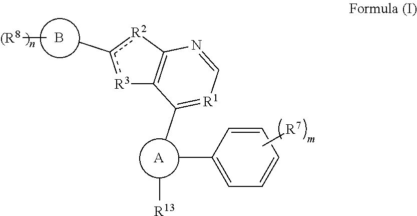

This application claims the benefit of U.S. Provisional Application Ser. No. 63/271,568 filed Oct. 25, 2021; U.S. Provisional Application Ser. No. 63/322,101 filed Mar. 21, 2022; and U.S. Provisional Application Ser. No. 63/343,290 filed May 18, 2022 which are hereby incorporated by reference in their entirety.

### BACKGROUND OF THE INVENTION

Activating mutations of epidermal growth factor receptor (EGFR) are one of the most common oncogenic drivers in cancers, such as non-small cell lung cancer (NSCLC). Three generations of EGFR tyrosine kinase inhibitors (TKIs) are used for the first-line treatment of subjects having NSCLC tumors that carrying EGFR-activating mutations, including gefitinib, erlotinib, and afatinib. These TKIs have been shown to be effective in the treatment of NSCLC tumors in subjects comprising certain EGFR mutations, including exon 19 deletion (Del19 or del19) and exon 21 point mutations, including the L858R point mutation. However, resistance to these TKIs develops in patients to which these drugs are administered, typically by development of an EGFR T790M point mutation in exon 20.

To overcome the resistance caused by T790M, third-generation TKIs that irreversibly interact with the cysteine 797 residue of EGFR (C797) and have more specificity for targeting T790M were developed, including osimertinib, CO-1686 (rociletinib), EGF816, WZ4002, and ASP8273. Osimertinib was approved as a second-line treatment to treat subjects having NSCLC whose tumors comprise the T790M mutation, and as a first-line treatment to treat subjects having NSCLC whose tumors comprise EGFR-activating mutations. Tumors in subjects treated with osimertinib may developed resistance by developing an EGFR C797S point mutation. The EGFR C797S mutation often appears in tumors in subjects in conjunction with the EGFR L858R and/or del19 mutations (e.g., L858R/C797S mutations and/or del19/C797S mutations). Therefore, there remains a need to develop inhibitors of EGFR comprising the L858R/C797S mutations and/or del19/C797S mutations for the treatment of subjects having cancer that is resistant to treatment with other TKIs such as osimertinib.

### SUMMARY OF THE INVENTION

Provided herein are compounds of Formula (XII):

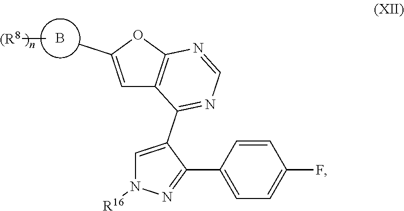

wherein:
B is aryl, heteroaryl, heterocycloalkyl, or C₃-C₁₂ cycloalkyl;
each R⁸ is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆ deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R¹⁶ is selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
n is 0, 1, 2, 3, 4, or 5; or a pharmaceutically acceptable salt thereof.

Also provided herein are compounds of Formula (I), (II), (IIa), (IIb), (IIc), (IId), (IIe), (III), (IV), (V), (VI), (VII), (VIII), (VIIIa), (IX), and (X), as defined herein.

In other embodiments are provided methods treating cancer in a subject, wherein the cancer has been determined to comprise a C797S mutation in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

In other embodiments are provided methods of treating metastatic cancer in a subject, wherein the metastatic cancer has been determined to comprise a C797S mutation in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

In other embodiments are provided methods of treating cancer in a subject, wherein the cancer has been determined to comprise L858R and C797S mutations in the epidermal growth factor receptor (EGFR) protein, comprising administering to the subject a composition comprising an effective amount of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

In other embodiments are provided methods of treating metastatic cancer in a subject, wherein the metastatic cancer has been determined to comprise exon 19 deletion and C797S mutations in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

Further provided herein are uses of a compound of Formula (I), or a pharmaceutically acceptable salt, solvate, or stereoisomer thereof, in the manufacture of a medicament for the treatment of cancer in a subject in need thereof. In some embodiments, the cancer in the subject expresses a C797S mutation in the epidermal growth factor receptor (EGFR) protein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

The present disclosure provides compounds according to Formula (I), or a pharmaceutically acceptable salt thereof,

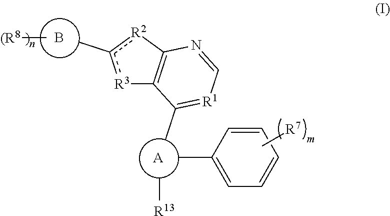

wherein:
A is heteroaryl;
B is C₆-C₁₀aryl, heteroaryl, heterocycloalkyl, or C₃-C₁₂ cycloalkyl;
R¹ is C—R⁹ or N;
R² is C—R¹⁰, N—R¹¹, O, or S;
R³ is C—R¹² or N—R⁴;
R⁷ and R⁸ are independently selected from deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹⁰, R¹¹, R¹², R¹³, and R¹⁴ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —S(O)(NH)R^{a}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a}; or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5;
provided that the compound of Formula (I) does not have formula

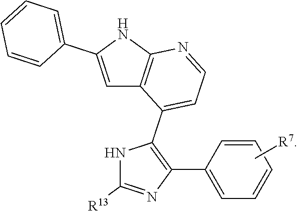

In other embodiments are provided compounds of Formula (I), wherein B is C₆-C₁₀aryl, or a pharmaceutically acceptable salt thereof. In other embodiments are provided compounds of Formula (I), or a pharmaceutically acceptable salt thereof. In other embodiments are provided compounds of Formula (I), wherein B is heteroaryl, or a pharmaceutically acceptable salt thereof. In other embodiments are provided compounds of Formula (I), wherein B is 5-membered or 6-membered heteroaryl, or a pharmaceutically acceptable salt thereof. In other embodiments are provided compounds of Formula (I), wherein B is 5-membered heteroaryl, or a pharmaceutically acceptable salt thereof. In other embodiments are provided compounds of Formula (I), wherein B is 6-membered heteroaryl, or a pharmaceutically acceptable salt thereof. In other embodiments are provided compounds of Formula (I), wherein B is heterocycloalkyl or C₃-C₁₂ cycloalkyl, or a pharmaceutically acceptable salt thereof. In other embodiments are provided compounds of Formula (I), or a pharmaceutically acceptable salt thereof, wherein R₁ is C—R₉. In other embodiments are provided compounds of Formula (I), or a pharmaceutically acceptable salt thereof, wherein R₁ is C—H. In other embodiments are provided compounds of Formula (I), or a pharmaceutically acceptable salt thereof, wherein R₁ is N. In other embodiments are provided compounds of Formula (I), or a pharmaceutically acceptable salt thereof, wherein A is 5-membered or 6-membered heteroaryl. In other embodiments are provided compounds of Formula (I), or a pharmaceutically acceptable salt thereof, wherein A is 5-membered heteroaryl. In other embodiments are provided compounds of Formula (I), or a pharmaceutically acceptable salt thereof, wherein A is selected from 1H-pyrazolyl, 1H-1,2,4-triazolyl, 1,3,4-oxadiazolyl, 2H-1,2,3-triazolyl, 1,3-oxazolyl, and 1,3-thiazolyl. In other embodiments are provided compounds of Formula (I), or a pharmaceutically acceptable salt thereof, wherein A is 6-membered heteroaryl. In other embodiments are provided compounds of Formula (I), or a pharmaceutically acceptable salt thereof, wherein A is pyridinyl or pyrimidinyl. In other embodiments are provided compounds of Formula (I), or a pharmaceutically acceptable salt thereof, wherein m is 1, 2, 3, 4, or 5. In other embodiments are provided compounds of Formula (I), or a pharmaceutically acceptable salt thereof, wherein m is 1. In other embodiments are provided compounds of Formula (I), or a pharmaceutically acceptable salt thereof, wherein R⁷ is halogen. In other embodiments are provided compounds of Formula (I), or a pharmaceutically acceptable salt thereof, wherein R¹ is fluoro, chloro, or bromo. In other embodiments are provided compounds of Formula (I), or a pharmaceutically acceptable salt thereof, wherein R⁷ is fluoro. In other embodiments are provided compounds of Formula (I), or a pharmaceutically acceptable salt thereof, wherein R⁷ is chloro. In other embodiments are provided compounds of Formula (I), or a pharmaceutically acceptable salt thereof, wherein R⁷ is bromo. In other embodiments are provided compounds of Formula (I), or a pharmaceutically acceptable salt thereof, wherein R⁷ is halogen in the 2-position. In other embodiments are provided compounds of Formula (I), wherein R⁷ is fluoro, chloro, or bromo. In other embodiments are provided compounds of Formula (I), or a pharmaceutically acceptable salt thereof, wherein m is 1 and R⁷ is fluoro. In other embodiments are provided compounds of Formula (I), or a pharmaceutically acceptable salt thereof, wherein R⁷ is halogen in the 3-position. In other embodiments are provided compounds of Formula (I), wherein R⁷ is fluoro, chloro, or bromo. In other embodiments are provided compounds of Formula (I), or a pharmaceutically acceptable salt thereof, wherein m is 1 and R⁷ is fluoro. In other embodiments are provided compounds of Formula (I), or a pharmaceutically acceptable salt thereof, wherein R⁷ is halogen in the 4-position. In other embodiments are provided compounds of Formula (I), wherein R⁷ is fluoro, chloro, or bromo. In other embodiments are provided compounds of Formula (I), or a pharmaceutically acceptable salt thereof, wherein m is 1 and R⁷ is fluoro. In other embodiments are provided compounds of Formula (I), or a pharmaceutically acceptable salt thereof, wherein R² is N—R¹¹, and compounds of Formula (I) wherein R¹¹ is hydrogen. In other embodiments are provided compounds of Formula (I), or a pharmaceutically acceptable salt thereof, wherein R² is O. In other embodiments are provided compounds of Formula (I), or a pharmaceutically acceptable salt thereof, wherein R² is S. In other embodiments are provided compounds of Formula (I), or a pharmaceutically acceptable salt thereof, wherein R² is N—R¹¹ and R³ is C—R¹², and compounds of Formula (I), wherein R¹¹ and R¹² are hydrogen. In other embodiments are provided compounds of Formula (I), or a pharmaceutically acceptable salt thereof, wherein R² is N—R¹¹ and R³ is N, including compounds of Formula (I) wherein R¹¹ is hydrogen.

In other embodiments are provided compounds of Formula (I), or a pharmaceutically acceptable salt thereof, wherein: A is 5-membered or 6-membered heteroaryl; B is C₆-C₁₀aryl or heteroaryl; R¹ is CH or N; R² is N—R¹¹, O, or S; R⁷ is halogen; R¹³ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; m is 1; and n is 0 or 1. In other embodiments are provided compounds of Formula (I), or a pharmaceutically acceptable salt thereof, wherein A is 5-membered heteroaryl. In other embodiments are provided compounds of Formula (I), or a pharmaceutically acceptable salt thereof, wherein A is selected from 1H-pyrazolyl, 1H-1,2,4-triazolyl, 1,3,4-oxadiazolyl, 2H-1,2,3-triazolyl, 1,3-oxazolyl, and 1,3-thiazolyl. In other embodiments are provided compounds of Formula (I), or a pharmaceutically acceptable salt thereof, wherein A is 6-membered heteroaryl. In other embodiments are provided compounds of Formula (I), or a pharmaceutically acceptable salt thereof, wherein A is pyridinyl or pyrimidinyl.

In other embodiments are provided compounds of Formula (I), or a pharmaceutically acceptable salt thereof, wherein: A is 5-membered or 6-membered heteroaryl; B is heteroaryl; R¹ is N; R² is O or S; and R³ is CH. In other embodiments are provided compounds of Formula (I), or a pharmaceutically acceptable salt thereof, wherein A is 5-membered heteroaryl. In other embodiments are provided compounds of Formula (I), or a pharmaceutically acceptable salt thereof, wherein A is selected from 1H-pyrazolyl, 1H-1,2,4-triazolyl, 1,3,4-oxadiazolyl, 2H-1,2,3-triazolyl, 1,3-oxazolyl, and 1,3-thiazolyl. In other embodiments are provided compounds of Formula (I), or a pharmaceutically acceptable salt thereof, wherein A is 6-membered heteroaryl. In other embodiments are provided compounds of Formula (I), or a pharmaceutically acceptable salt thereof, wherein R² is O. In other embodiments are provided compounds of Formula (I), or a pharmaceutically acceptable salt thereof, wherein R² is S.

In other embodiments are provided compounds of Formula (I), or a pharmaceutically acceptable salt thereof, wherein: A is 5-membered or 6-membered heteroaryl; B is heterocycloalkyl; R¹ is N; R² is O or S; and R³ is CH. In other embodiments are provided compounds of Formula (I), or a pharmaceutically acceptable salt thereof, wherein A is 5-membered heteroaryl. In other embodiments are provided compounds of Formula (I), or a pharmaceutically acceptable salt thereof, wherein A is selected from 1H-pyrazolyl, 1H-1,2,4-triazolyl, 1,3,4-oxadiazolyl, 2H-1,2,3-triazolyl, 1,3-oxazolyl, and 1,3-thiazolyl. In other embodiments are provided compounds of Formula (I), or a pharmaceutically acceptable salt thereof, wherein A is 6-membered heteroaryl. In other embodiments are provided compounds of Formula (I), or a pharmaceutically acceptable salt thereof, wherein R² is O. In other embodiments are provided compounds of Formula (I), or a pharmaceutically acceptable salt thereof, wherein R² is S. In other embodiments are provided compounds of Formula (I), or a pharmaceutically acceptable salt thereof, wherein: A is 5-membered heteroaryl; B is C₆-C₁₀aryl; R¹ is N; R² is NH or 0; R³ is CH or N; and R⁷ is 4-fluoro, 4-chloro, or 4-bromo. In other embodiments are provided compounds of Formula (I), or a pharmaceutically acceptable salt thereof, wherein: A is pyrazolyl; B is phenyl; R² is NH; and R³ is CH. In other embodiments are provided compounds of Formula (I), or a pharmaceutically acceptable salt thereof, wherein: A is pyrazolyl; B is phenyl; R² is NH; and R³ is CH. In other embodiments are provided compounds of Formula (I), or a pharmaceutically acceptable salt thereof, wherein: A is pyrazolyl; B is phenyl; R² is 0; and R³ is N. In other embodiments are provided compounds of Formula (I), or a pharmaceutically acceptable salt thereof, wherein n is 0. In other embodiments are provided compounds of Formula (I), or a pharmaceutically acceptable salt thereof, wherein n is 1.

In other embodiments are provided compounds of Formula (I), or a pharmaceutically acceptable salt thereof, wherein: A is 5-membered or 6-membered heteroaryl; B is phenyl; R¹ is N; R² is O or S; and R³ is CH. In other embodiments are provided compounds of Formula (I), or a pharmaceutically acceptable salt thereof, wherein A is 5-membered heteroaryl. In other embodiments are provided compounds of Formula (I), or a pharmaceutically acceptable salt thereof, wherein A is selected from 1H-pyrazolyl, 1H-1,2,4-triazolyl, 1,3,4-oxadiazolyl, 2H-1,2,3-triazolyl, 1,3-oxazolyl, and 1,3-thiazolyl. In other embodiments are provided compounds of Formula (I), or a pharmaceutically acceptable salt thereof, wherein A is 6-membered heteroaryl. In other embodiments are provided compounds of Formula (I), or a pharmaceutically acceptable salt thereof, wherein R² is 0. In other embodiments are provided compounds of Formula (I), or a pharmaceutically acceptable salt thereof, wherein R² is S.

In other embodiments are provided compounds of Formula (Ia), or a pharmaceutically acceptable salt thereof,

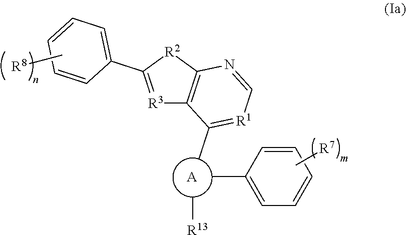

wherein:
A is heteroaryl;
R¹ is C—R⁹ or N;
R² is N—R¹¹, O, or S;
R³ is C—R¹² or N;
R⁷ and R⁸ are independently selected from deuterium, halogen, —CN, NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², and R¹³ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —S(O)(NH)R^{a}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5;
provided that the compound of Formula (Ia) does not have formula

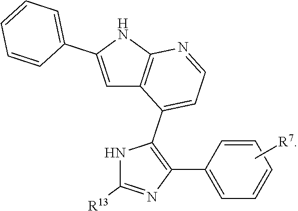

In other embodiments are provided compounds of Formula (Ia), or a pharmaceutically acceptable salt thereof, wherein R¹ is C—R⁹. In other embodiments are provided compounds of Formula (Ia), or a pharmaceutically acceptable salt thereof, wherein R¹ is C—H. In other embodiments are provided compounds of Formula (Ia), or a pharmaceutically acceptable salt thereof, wherein R¹ is N. In other embodiments are provided compounds of Formula (Ia), or a pharmaceutically acceptable salt thereof, wherein A is 5-membered or 6-membered heteroaryl. In other embodiments are provided compounds of Formula (Ia), or a pharmaceutically acceptable salt thereof, wherein A is 5-membered heteroaryl. In other embodiments are provided compounds of Formula (Ia), or a pharmaceutically acceptable salt thereof, wherein A is selected from 1H-pyrazolyl, 1H-1,2,4-triazolyl, 1,3,4-oxadiazolyl, 2H-1,2,3-triazolyl, 1,3-oxazolyl, and 1,3-thiazolyl.

In other embodiments are provided compounds of Formula (Ia), or a pharmaceutically acceptable salt thereof, wherein A is 6-membered heteroaryl. In other embodiments are provided compounds of Formula (Ia), or a pharmaceutically acceptable salt thereof, wherein A is pyridinyl or pyrimidinyl. In other embodiments are provided compounds of Formula (Ia), or a pharmaceutically acceptable salt thereof, wherein m is 1, 2, 3, 4, or 5. In other embodiments are provided compounds of Formula (Ia), or a pharmaceutically acceptable salt thereof, wherein m is 1. In other embodiments are provided compounds of Formula (Ia), or a pharmaceutically acceptable salt thereof, wherein R⁷ is halogen. In other embodiments are provided compounds of Formula (Ia), or a pharmaceutically acceptable salt thereof, wherein R⁷ is fluoro, chloro, or bromo. In other embodiments are provided compounds of Formula (Ia), or a pharmaceutically acceptable salt thereof, wherein R⁷ is fluoro. In other embodiments are provided compounds of Formula (Ia), or a pharmaceutically acceptable salt thereof, wherein R⁷ is chloro. In other embodiments are provided compounds of Formula (Ia), or a pharmaceutically acceptable salt thereof, wherein R⁷ is bromo. In other embodiments are provided compounds of Formula (Ia), or a pharmaceutically acceptable salt thereof, wherein R⁷ is halogen in the 2-position. In other embodiments are provided compounds of Formula (Ia), wherein R⁷ is fluoro, chloro, or bromo. In other embodiments are provided compounds of Formula (Ia), or a pharmaceutically acceptable salt thereof, wherein m is 1 and R⁷ is fluoro. In other embodiments are provided compounds of Formula (Ia), or a pharmaceutically acceptable salt thereof, wherein R⁷ is halogen in the 3-position. In other embodiments are provided compounds of Formula (Ia), wherein R⁷ is fluoro, chloro, or bromo. In other embodiments are provided compounds of Formula (Ia), or a pharmaceutically acceptable salt thereof, wherein m is 1 and R⁷ is fluoro. In other embodiments are provided compounds of Formula (Ia), or a pharmaceutically acceptable salt thereof, wherein R⁷ is halogen in the 4-position. In other embodiments are provided compounds of Formula (Ia), wherein R⁷ is fluoro, chloro, or bromo. In other embodiments are provided compounds of Formula (Ia), or a pharmaceutically acceptable salt thereof, wherein m is 1 and R⁷ is fluoro. In other embodiments are provided compounds of Formula (Ia), or a pharmaceutically acceptable salt thereof, wherein R² is N—R¹¹. In other embodiments are provided compounds of Formula (Ia), or a pharmaceutically acceptable salt thereof, wherein R¹¹ is hydrogen. In other embodiments are provided compounds of Formula (Ia), or a pharmaceutically acceptable salt thereof, wherein R² is O. In other embodiments are provided compounds of Formula (Ia), or a pharmaceutically acceptable salt thereof, wherein R² is S. In other embodiments are provided compounds of Formula (Ia), or a pharmaceutically acceptable salt thereof, wherein R² is N—R¹¹ and R³ is C—R¹². In other embodiments are provided compounds of Formula (Ia), or a pharmaceutically acceptable salt thereof, wherein R¹¹ and R¹² are hydrogen. In other embodiments are provided compounds of Formula (Ia), or a pharmaceutically acceptable salt thereof, wherein R² is N—R¹¹ and R³ is N. In other embodiments are provided compounds of Formula (Ia), or a pharmaceutically acceptable salt thereof, wherein R¹¹ is hydrogen.

In other embodiments are provided compounds of Formula (Ia), or a pharmaceutically acceptable salt thereof, wherein: A is 5-membered or 6-membered heteroaryl; R¹ is CH or N; R² is N—R¹¹, O, or S; R⁷ is halogen; R¹³ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; m is 1; and n is 0 or 1. In other embodiments are provided compounds of Formula (Ia), or a pharmaceutically acceptable salt thereof, wherein A is 5-membered heteroaryl. In other embodiments are provided compounds of Formula (Ia), or a pharmaceutically acceptable salt thereof, wherein A is selected from 1H-pyrazolyl, 1H-1,2,4-triazolyl, 1,3,4-oxadiazolyl, 2H-1,2,3-triazolyl, 1,3-oxazolyl, and 1,3-thiazolyl. In other embodiments are provided compounds of Formula (Ia), or a pharmaceutically acceptable salt thereof, wherein A is 6-membered heteroaryl. In other embodiments are provided compounds of Formula (Ia), or a pharmaceutically acceptable salt thereof, wherein A is pyridinyl or pyrimidinyl.

In other embodiments are provided compounds of Formula (Ia), or a pharmaceutically acceptable salt thereof, wherein: A is 5-membered heteroaryl; R¹ is N; R² is NH or 0; R³ is CH or N; and R⁷ is 4-fluoro, 4-chloro, or 4-bromo. In other embodiments are provided compounds of Formula (Ia), or a pharmaceutically acceptable salt thereof, wherein: A is pyrazolyl; and R³ is CH. In other embodiments are provided compounds of Formula (Ia), or a pharmaceutically acceptable salt thereof, wherein: A is pyrazolyl; and R³ is N. In other embodiments are provided compounds of Formula (Ia), or a pharmaceutically acceptable salt thereof, wherein n is 0. In other embodiments are provided compounds of Formula (Ia), or a pharmaceutically acceptable salt thereof, wherein n is 1.

In other embodiments are provided compounds of Formula (Ia), or a pharmaceutically acceptable salt thereof, wherein: A is 5-membered or 6-membered heteroaryl; R¹ is N; R² is O or S; and R³ is CH. In other embodiments are provided compounds of Formula (Ia), or a pharmaceutically acceptable salt thereof, wherein A is 5-membered heteroaryl. In other embodiments are provided compounds of Formula (Ia), or a pharmaceutically acceptable salt thereof, wherein A is selected from 1H-pyrazolyl, 1H-1,2,4-triazolyl, 1,3,4-oxadiazolyl, 2H-1,2,3-triazolyl, 1,3-oxazolyl, and 1,3-thiazolyl. In other embodiments are provided compounds of Formula (Ia), or a pharmaceutically acceptable salt thereof, wherein A is 6-membered heteroaryl. In other embodiments are provided compounds of Formula (Ia), or a pharmaceutically acceptable salt thereof, wherein R² is 0. In other embodiments are provided compounds of Formula (Ia), or a pharmaceutically acceptable salt thereof, wherein R² is S.

In other embodiments are provided compounds of Formula (II), or a pharmaceutically acceptable salt thereof,

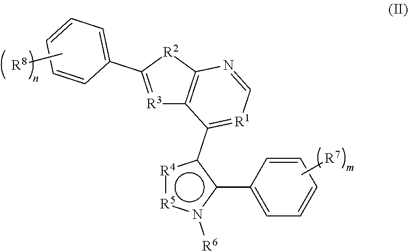

wherein:
R¹ is C—R⁹ or N;
R² is N—R¹¹, O, or S;
R³ is C—R¹² or N;
R⁴ is C—R¹³, N, N—R¹⁴, O, or S;
R³ is C—R¹¹, N, or N—R¹⁷;
R⁶ is absent or when present is R^{a};
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², R¹³, R¹⁴, R¹, and R¹⁷ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5;
provided that the compound of Formula (II) does not have formula

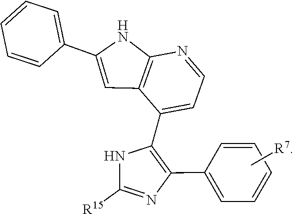

In other embodiments are provided compounds of Formula (II), or a pharmaceutically acceptable salt thereof, wherein R¹ is N, R² is O or S; and R³ is CH. In other embodiments are provided compounds of Formula (II), or a pharmaceutically acceptable salt thereof, wherein R¹ is N, R² is O; and R³ is CH. In other embodiments are provided compounds of Formula (II), or a pharmaceutically acceptable salt thereof, wherein R¹ is N, R² is S; and R³ is CH.

In other embodiments are provided compounds of Formula (II), or a pharmaceutically acceptable salt thereof, wherein m is 1, 2, 3, 4, or 5. In other embodiments are provided compounds of Formula (II), or a pharmaceutically acceptable salt thereof, wherein m is 1. In other embodiments are provided compounds of Formula (II), or a pharmaceutically acceptable salt thereof, wherein R⁷ is halogen. In other embodiments are provided compounds of Formula (II), or a pharmaceutically acceptable salt thereof, wherein R⁷ is fluoro, chloro, or bromo. In other embodiments are provided compounds of Formula (II), or a pharmaceutically acceptable salt thereof, wherein R⁷ is fluoro. In other embodiments are provided compounds of Formula (II), or a pharmaceutically acceptable salt thereof, wherein R⁷ is chloro. In other embodiments are provided compounds of Formula (II), or a pharmaceutically acceptable salt thereof, wherein R⁷ is bromo. In other embodiments are provided compounds of Formula (II), or a pharmaceutically acceptable salt thereof, wherein R⁷ is halogen in the 2-position. In other embodiments are provided compounds of Formula (II), wherein R⁷ is fluoro, chloro, or bromo. In other embodiments are provided compounds of Formula (II), or a pharmaceutically acceptable salt thereof, wherein m is 1 and R⁷ is fluoro. In other embodiments are provided compounds of Formula (II), or a pharmaceutically acceptable salt thereof, wherein R⁷ is halogen in the 3-position. In other embodiments are provided compounds of Formula (II), wherein R⁷ is fluoro, chloro, or bromo. In other embodiments are provided compounds of Formula (II), or a pharmaceutically acceptable salt thereof, wherein m is 1 and R⁷ is fluoro. In other embodiments are provided compounds of Formula (II), or a pharmaceutically acceptable salt thereof, wherein R⁷ is halogen in the 4-position. In other embodiments are provided compounds of Formula (II), wherein R⁷ is fluoro, chloro, or bromo. In other embodiments are provided compounds of Formula (II), or a pharmaceutically acceptable salt thereof, wherein m is 1 and R⁷ is fluoro. In other embodiments are provided compounds of Formula (II), or a pharmaceutically acceptable salt thereof, wherein R² is N—R¹¹. In other embodiments are provided compounds of Formula (II), or a pharmaceutically acceptable salt thereof, wherein R¹¹ is hydrogen. In other embodiments are provided compounds of Formula (II), or a pharmaceutically acceptable salt thereof, wherein R² is O. In other embodiments are provided compounds of Formula (II), or a pharmaceutically acceptable salt thereof, wherein R² is S. In other embodiments are provided compounds of Formula (II), or a pharmaceutically acceptable salt thereof, wherein R² is N—R¹¹ and R³ is C—R¹². In other embodiments are provided compounds of Formula (II), or a pharmaceutically acceptable salt thereof, wherein R¹¹ and R¹² are hydrogen. In other embodiments are provided compounds of Formula (II), or a pharmaceutically acceptable salt thereof, wherein R² is N—R¹¹ and R³ is N. In other embodiments are provided compounds of Formula (II), or a pharmaceutically acceptable salt thereof, wherein R¹¹ is hydrogen.

In other embodiments are provided compounds of Formula (II), or a pharmaceutically acceptable salt thereof, wherein: R¹ is CH or N; R² is NH, O, or S; R⁷ is halogen; R⁶ is R^{a}; m is 1; and n is 0 or 1.

In other embodiments are provided compounds of Formula (II), or a pharmaceutically acceptable salt thereof, wherein: R¹ is CH or N; R² is NH, O, or S; R⁷ is halogen; R⁶ is absent; m is 1; and n is 0 or 1.

In other embodiments are provided compounds of Formula (II), or a pharmaceutically acceptable salt thereof, wherein: R¹ is N; R² is NH or O; R³ is CH or N; and R⁷ is 4-fluoro, 4-chloro, or 4-bromo. In other embodiments are provided compounds of Formula (II), or a pharmaceutically acceptable salt thereof, wherein R² is NH, and R³ is CH. In other embodiments are provided compounds of Formula (II), or a pharmaceutically acceptable salt thereof, wherein R² is NH, and R³ is N. In other embodiments are provided compounds of Formula (II), or a pharmaceutically acceptable salt thereof, wherein R² is O, and R³ is CH. In other embodiments are provided compounds of Formula (II), or a pharmaceutically acceptable salt thereof, wherein R² is O, and R³ is N.

In other embodiments are provided compounds of Formula (II), or a pharmaceutically acceptable salt thereof, wherein n is 0.

In other embodiments are provided compounds of Formula (II), or a pharmaceutically acceptable salt thereof, wherein n is 1.

Also provided herein are compounds of Formula (IIa),

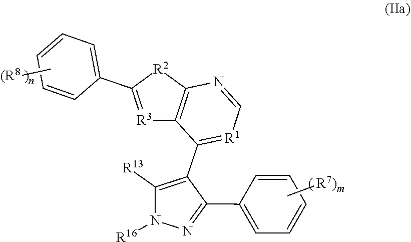

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², R¹³, and R¹⁶ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5;
or a pharmaceutically acceptable salt thereof.

In other embodiments are provided compounds of Formula (IIa), or a pharmaceutically acceptable salt thereof, wherein R¹ is N, R² is 0 or S; and R³ is CH. In other embodiments are provided compounds of Formula (IIa), or a pharmaceutically acceptable salt thereof, wherein R¹ is N, R² is 0; and R³ is CH. In other embodiments are provided compounds of Formula (IIa), or a pharmaceutically acceptable salt thereof, wherein R¹ is N, R² is S; and R³ is CH.

In other embodiments are provided compounds of Formula (IIa), or a pharmaceutically acceptable salt thereof, wherein R¹ is C—R⁹. In other embodiments are provided compounds of Formula (IIa), or a pharmaceutically acceptable salt thereof, wherein R¹ is C—H. In other embodiments are provided compounds of Formula (IIa), or a pharmaceutically acceptable salt thereof, wherein R¹ is N. In other embodiments are provided compounds of Formula (IIa), or a pharmaceutically acceptable salt thereof, wherein m is 1, 2, 3, 4, or 5. In other embodiments are provided compounds of Formula (IIa), or a pharmaceutically acceptable salt thereof, wherein m is 1. In other embodiments are provided compounds of Formula (IIa), or a pharmaceutically acceptable salt thereof, wherein R⁷ is halogen. In other embodiments are provided compounds of Formula (IIa), or a pharmaceutically acceptable salt thereof, wherein R⁷ is fluoro, chloro, or bromo. In other embodiments are provided compounds of Formula (IIa), or a pharmaceutically acceptable salt thereof, wherein R⁷ is fluoro. In other embodiments are provided compounds of Formula (IIa), or a pharmaceutically acceptable salt thereof, wherein R⁷ is chloro. In other embodiments are provided compounds of Formula (IIa), or a pharmaceutically acceptable salt thereof, wherein R⁷ is bromo. In other embodiments are provided compounds of Formula (IIa), or a pharmaceutically acceptable salt thereof, wherein R⁷ is halogen in the 2-position. In other embodiments are provided compounds of Formula (IIa), wherein R⁷ is fluoro, chloro, or bromo. In other embodiments are provided compounds of Formula (IIa), or a pharmaceutically acceptable salt thereof, wherein m is 1 and R⁷ is fluoro. In other embodiments are provided compounds of Formula (IIa), or a pharmaceutically acceptable salt thereof, wherein R⁷ is halogen in the 3-position. In other embodiments are provided compounds of Formula (IIa), wherein R⁷ is fluoro, chloro, or bromo. In other embodiments are provided compounds of Formula (IIa), or a pharmaceutically acceptable salt thereof, wherein m is 1 and R⁷ is fluoro. In other embodiments are provided compounds of Formula (IIa), or a pharmaceutically acceptable salt thereof, wherein R⁷ is halogen in the 4-position. In other embodiments are provided compounds of Formula (IIa), wherein R⁷ is fluoro, chloro, or bromo. In other embodiments are provided compounds of Formula (IIa), or a pharmaceutically acceptable salt thereof, wherein m is 1 and R⁷ is fluoro. In other embodiments are provided compounds of Formula (IIa), or a pharmaceutically acceptable salt thereof, wherein R² is N—R¹¹. In other embodiments are provided compounds of Formula (IIa), or a pharmaceutically acceptable salt thereof, wherein R¹¹ is hydrogen. In other embodiments are provided compounds of Formula (IIa), or a pharmaceutically acceptable salt thereof, wherein R² is O. In other embodiments are provided compounds of Formula (IIa), or a pharmaceutically acceptable salt thereof, wherein R² is S. In other embodiments are provided compounds of Formula (IIa), or a pharmaceutically acceptable salt thereof, wherein R² is N—R¹¹ and R³ is C—R¹². In other embodiments are provided compounds of Formula (IIa), or a pharmaceutically acceptable salt thereof, wherein R¹¹ and R¹² are hydrogen. In other embodiments are provided compounds of Formula (IIa), or a pharmaceutically acceptable salt thereof, wherein R² is N—R¹¹ and R³ is N. In other embodiments are provided compounds of Formula (IIa), or a pharmaceutically acceptable salt thereof, wherein R¹¹ is hydrogen.

In other embodiments are provided compounds of Formula (IIa), or a pharmaceutically acceptable salt thereof, wherein: R¹ is CH or N; R² is N—R¹¹, O, or S; R⁷ is halogen; R¹³ is hydrogen; R¹⁶ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; m is 1; and n is 0 or 1.

In other embodiments are provided compounds of Formula (IIa), or a pharmaceutically acceptable salt thereof, wherein: R¹ is N; R² is NH or 0; R³ is CH or N; and R⁷ is 4-fluoro, 4-chloro, or 4-bromo.

In other embodiments are provided compounds of Formula (IIa), or a pharmaceutically acceptable salt thereof, wherein: R¹ is N; R² is NH; and R³ is CH.

In other embodiments are provided compounds of Formula (IIa), or a pharmaceutically acceptable salt thereof, wherein: R¹ is CH; R² is NH; and R³ is CH.

In other embodiments are provided compounds of Formula (IIa), or a pharmaceutically acceptable salt thereof, wherein: R¹ is N; R² is NH; and R³ is N.

In other embodiments are provided compounds of Formula (IIa), or a pharmaceutically acceptable salt thereof, wherein: R¹ is 0; R² is NH; and R³ is CH.

In other embodiments are provided compounds of Formula (IIa), or a pharmaceutically acceptable salt thereof, wherein: R¹ is 0; R² is NH; and R³ is CH.

In other embodiments are provided compounds of Formula (IIa), or a pharmaceutically acceptable salt thereof, wherein: R¹ is 0; R² is NH; and R³ is N.

In other embodiments are provided compounds of Formula (IIa), or a pharmaceutically acceptable salt thereof, wherein n is 0.

In other embodiments are provided compounds of Formula (IIa), or a pharmaceutically acceptable salt thereof, wherein n is 1.

A compound according to Formula (IIb), or a pharmaceutically acceptable salt thereof,

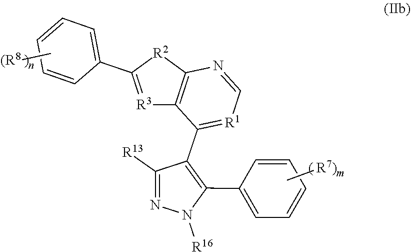

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁶, R⁹, R¹¹, R¹², and R¹³ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5.

In other embodiments are provided compounds of Formula (IIb), or a pharmaceutically acceptable salt thereof, wherein R¹ is N, R² is O or S; and R³ is CH. In other embodiments are provided compounds of Formula (IIb), or a pharmaceutically acceptable salt thereof, wherein R¹ is N, R² is O; and R³ is CH. In other embodiments are provided compounds of Formula (IIb), or a pharmaceutically acceptable salt thereof, wherein R¹ is N, R² is S; and R³ is CH.

In other embodiments are provided compounds of Formula (IIb), or a pharmaceutically acceptable salt thereof, wherein R¹ is C—R⁹. In other embodiments are provided compounds of Formula (IIb), or a pharmaceutically acceptable salt thereof, wherein R¹ is C—H. In other embodiments are provided compounds of Formula (IIb), or a pharmaceutically acceptable salt thereof, wherein R¹ is N. In other embodiments are provided compounds of Formula (IIb), or a pharmaceutically acceptable salt thereof, wherein m is 1, 2, 3, 4, or 5. In other embodiments are provided compounds of Formula (IIb), or a pharmaceutically acceptable salt thereof, wherein m is 1. In other embodiments are provided compounds of Formula (IIb), or a pharmaceutically acceptable salt thereof, wherein R⁷ is halogen. In other embodiments are provided compounds of Formula (IIb), or a pharmaceutically acceptable salt thereof, wherein R⁷ is fluoro, chloro, or bromo. In other embodiments are provided compounds of Formula (IIb), or a pharmaceutically acceptable salt thereof, wherein R⁷ is fluoro. In other embodiments are provided compounds of Formula (IIb), or a pharmaceutically acceptable salt thereof, wherein R⁷ is chloro. In other embodiments are provided compounds of Formula (IIb), or a pharmaceutically acceptable salt thereof, wherein R⁷ is bromo. In other embodiments are provided compounds of Formula (IIb), or a pharmaceutically acceptable salt thereof, wherein R⁷ is halogen in the 2-position. In other embodiments are provided compounds of Formula (IIb), wherein R⁷ is fluoro, chloro, or bromo. In other embodiments are provided compounds of Formula (IIb), or a pharmaceutically acceptable salt thereof, wherein m is 1 and R⁷ is fluoro. In other embodiments are provided compounds of Formula (IIb), or a pharmaceutically acceptable salt thereof, wherein R⁷ is halogen in the 3-position. In other embodiments are provided compounds of Formula (IIb), wherein R⁷ is fluoro, chloro, or bromo. In other embodiments are provided compounds of Formula (IIb), or a pharmaceutically acceptable salt thereof, wherein m is 1 and R⁷ is fluoro. In other embodiments are provided compounds of Formula (IIb), or a pharmaceutically acceptable salt thereof, wherein R⁷ is halogen in the 4-position. In other embodiments are provided compounds of Formula (IIb), wherein R⁷ is fluoro, chloro, or bromo. In other embodiments are provided compounds of Formula (IIb), or a pharmaceutically acceptable salt thereof, wherein m is 1 and R⁷ is fluoro. In other embodiments are provided compounds of Formula (IIb), or a pharmaceutically acceptable salt thereof, wherein R² is N—R¹¹. In other embodiments are provided compounds of Formula (IIb), or a pharmaceutically acceptable salt thereof, wherein R¹¹ is hydrogen. In other embodiments are provided compounds of Formula (IIb), or a pharmaceutically acceptable salt thereof, wherein R² is O. In other embodiments are provided compounds of Formula (IIb), or a pharmaceutically acceptable salt thereof, wherein R² is S. In other embodiments are provided compounds of Formula (IIb), or a pharmaceutically acceptable salt thereof, wherein R² is N—R¹¹ and R³ is C—R¹². In other embodiments are provided compounds of Formula (IIb), or a pharmaceutically acceptable salt thereof, wherein R¹¹ and R¹² are hydrogen. In other embodiments are provided compounds of Formula (IIb), or a pharmaceutically acceptable salt thereof, wherein R² is N—R¹¹ and R³ is N. In other embodiments are provided compounds of Formula (IIb), or a pharmaceutically acceptable salt thereof, wherein R¹¹ is hydrogen.

In other embodiments are provided compounds of Formula (IIb), or a pharmaceutically acceptable salt thereof, wherein: R¹ is CH or N; R² is NH, O, or S; R⁶ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; R⁷ is halogen; R¹³ is hydrogen; m is 1; and n is 0 or 1.

In other embodiments are provided compounds of Formula (IIb), or a pharmaceutically acceptable salt thereof, wherein: R¹ is N; R² is NH or 0; and R³ is CH or N.

In other embodiments are provided compounds of Formula (IIb), or a pharmaceutically acceptable salt thereof, wherein: R¹ is N; R² is NH; and R³ is CH.

In other embodiments are provided compounds of Formula (IIb), or a pharmaceutically acceptable salt thereof, wherein: R¹ is CH; R² is NH; and R³ is CH.

In other embodiments are provided compounds of Formula (IIb), or a pharmaceutically acceptable salt thereof, wherein: R¹ is N; R² is NH; and R³ is N.

In other embodiments are provided compounds of Formula (IIb), or a pharmaceutically acceptable salt thereof, wherein: R¹ is 0; R² is NH; and R³ is CH.

In other embodiments are provided compounds of Formula (IIb), or a pharmaceutically acceptable salt thereof, wherein: R¹ is 0; R² is NH; and R³ is N.

In other embodiments are provided compounds of Formula (IIb), or a pharmaceutically acceptable salt thereof, wherein n is 0.

In other embodiments are provided compounds of Formula (IIb), or a pharmaceutically acceptable salt thereof, wherein n is 1.

Also provided herein are compounds according to Formula (IIc), or a pharmaceutically acceptable salt thereof,

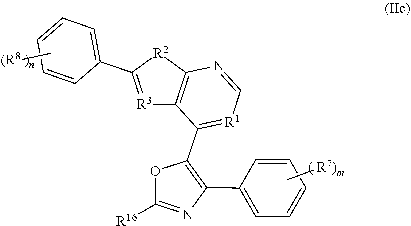

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², and R¹¹ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5.

In other embodiments are provided compounds of Formula (IIc), or a pharmaceutically acceptable salt thereof, wherein R¹ is N, R² is 0 or S; and R³ is CH. In other embodiments are provided compounds of Formula (IIc), or a pharmaceutically acceptable salt thereof, wherein R¹ is N, R² is 0; and R³ is CH. In other embodiments are provided compounds of Formula (IIc), or a pharmaceutically acceptable salt thereof, wherein R¹ is N, R² is S; and R³ is CH.

In other embodiments are provided compounds of Formula (IIc), or a pharmaceutically acceptable salt thereof, wherein R¹ is C—R⁹. In other embodiments are provided compounds of Formula (IIc), or a pharmaceutically acceptable salt thereof, wherein R¹ is C—H. In other embodiments are provided compounds of Formula (IIc), or a pharmaceutically acceptable salt thereof, wherein R¹ is N. In other embodiments are provided compounds of Formula (IIc), or a pharmaceutically acceptable salt thereof, wherein m is 1, 2, 3, 4, or 5. In other embodiments are provided compounds of Formula (IIc), or a pharmaceutically acceptable salt thereof, wherein m is 1. In other embodiments are provided compounds of Formula (IIc), or a pharmaceutically acceptable salt thereof, wherein R⁷ is halogen. In other embodiments are provided compounds of Formula (IIc), or a pharmaceutically acceptable salt thereof, wherein R⁷ is fluoro, chloro, or bromo. In other embodiments are provided compounds of Formula (IIc), or a pharmaceutically acceptable salt thereof, wherein R⁷ is fluoro. In other embodiments are provided compounds of Formula (IIc), or a pharmaceutically acceptable salt thereof, wherein R⁷ is chloro. In other embodiments are provided compounds of Formula (IIc), or a pharmaceutically acceptable salt thereof, wherein R⁷ is bromo. In other embodiments are provided compounds of Formula (IIc), or a pharmaceutically acceptable salt thereof, wherein R⁷ is halogen in the 2-position. In other embodiments are provided compounds of Formula (IIc), wherein R⁷ is fluoro, chloro, or bromo. In other embodiments are provided compounds of Formula (IIc), or a pharmaceutically acceptable salt thereof, wherein m is 1 and R⁷ is fluoro. In other embodiments are provided compounds of Formula (IIc), or a pharmaceutically acceptable salt thereof, wherein R⁷ is halogen in the 3-position. In other embodiments are provided compounds of Formula (IIc), wherein R⁷ is fluoro, chloro, or bromo. In other embodiments are provided compounds of Formula (IIc), or a pharmaceutically acceptable salt thereof, wherein m is 1 and R⁷ is fluoro. In other embodiments are provided compounds of Formula (IIc), or a pharmaceutically acceptable salt thereof, wherein R⁷ is halogen in the 4-position. In other embodiments are provided compounds of Formula (IIc), wherein R⁷ is fluoro, chloro, or bromo. In other embodiments are provided compounds of Formula (IIc), or a pharmaceutically acceptable salt thereof, wherein m is 1 and R⁷ is fluoro. In other embodiments are provided compounds of Formula (IIc), or a pharmaceutically acceptable salt thereof, wherein R² is N—R¹¹. In other embodiments are provided compounds of Formula (IIc), or a pharmaceutically acceptable salt thereof, wherein R¹¹ is hydrogen. In other embodiments are provided compounds of Formula (IIc), or a pharmaceutically acceptable salt thereof, wherein R² is O. In other embodiments are provided compounds of Formula (IIc), or a pharmaceutically acceptable salt thereof, wherein R² is S. In other embodiments are provided compounds of Formula (IIc), or a pharmaceutically acceptable salt thereof, wherein R² is N—R¹¹ and R³ is C—R¹². In other embodiments are provided compounds of Formula (IIc), or a pharmaceutically acceptable salt thereof, wherein R¹¹ and R¹² are hydrogen. In other embodiments are provided compounds of Formula (IIc), or a pharmaceutically acceptable salt thereof, wherein R² is N—R¹¹ and R³ is N. In other embodiments are provided compounds of Formula (IIc), or a pharmaceutically acceptable salt thereof, wherein R¹¹ is hydrogen.

In other embodiments are provided compounds of Formula (IIc), or a pharmaceutically acceptable salt thereof, wherein: R¹ is CH or N; R² is NH, O, or S; R⁷ is halogen; R¹¹ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; m is 1; and n is 0 or 1.

In other embodiments are provided compounds of Formula (IIc), or a pharmaceutically acceptable salt thereof, wherein: R¹ is N; R² is NH or 0; and R³ is CH or N.

In other embodiments are provided compounds of Formula (IIc), or a pharmaceutically acceptable salt thereof, wherein: R¹ is N; R² is NH; and R³ is CH.

In other embodiments are provided compounds of Formula (IIc), or a pharmaceutically acceptable salt thereof, wherein: R¹ is CH; R² is NH; and R³ is CH.

In other embodiments are provided compounds of Formula (IIc), or a pharmaceutically acceptable salt thereof, wherein: R¹ is N; R² is NH; and R³ is N.

In other embodiments are provided compounds of Formula (IIc), or a pharmaceutically acceptable salt thereof, wherein: R¹ is 0; R² is NH; and R³ is CH.

In other embodiments are provided compounds of Formula (IIc), or a pharmaceutically acceptable salt thereof, wherein: R¹ is 0; R² is NH; and R³ is N.

In other embodiments are provided compounds of Formula (IIc), or a pharmaceutically acceptable salt thereof, wherein n is 0.

In other embodiments are provided compounds of Formula (IIc), or a pharmaceutically acceptable salt thereof, wherein n is 1.

Also provided herein are compounds of Formula (IId), or a pharmaceutically acceptable salt thereof,

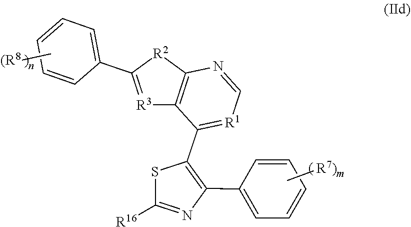

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², and R¹⁵ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5.

In other embodiments are provided compounds of Formula (IId), or a pharmaceutically acceptable salt thereof, wherein R¹ is N, R² is O or S; and R³ is CH. In other embodiments are provided compounds of Formula (IId), or a pharmaceutically acceptable salt thereof, wherein R¹ is N, R² is O; and R³ is CH. In other embodiments are provided compounds of Formula (IId), or a pharmaceutically acceptable salt thereof, wherein R¹ is N, R² is S; and R³ is CH.

In other embodiments are provided compounds of Formula (IId), or a pharmaceutically acceptable salt thereof, wherein R¹ is C—R⁹. In other embodiments are provided compounds of Formula (IId), or a pharmaceutically acceptable salt thereof, wherein R¹ is C—H. In other embodiments are provided compounds of Formula (IId), or a pharmaceutically acceptable salt thereof, wherein R¹ is N. In other embodiments are provided compounds of Formula (IId), or a pharmaceutically acceptable salt thereof, wherein m is 1, 2, 3, 4, or 5. In other embodiments are provided compounds of Formula (IId), or a pharmaceutically acceptable salt thereof, wherein m is 1. In other embodiments are provided compounds of Formula (IId), or a pharmaceutically acceptable salt thereof, wherein R⁷ is halogen. In other embodiments are provided compounds of Formula (IId), or a pharmaceutically acceptable salt thereof, wherein R⁷ is fluoro, chloro, or bromo. In other embodiments are provided compounds of Formula (IId), or a pharmaceutically acceptable salt thereof, wherein R⁷ is fluoro. In other embodiments are provided compounds of Formula (IId), or a pharmaceutically acceptable salt thereof, wherein R⁷ is chloro. In other embodiments are provided compounds of Formula (IId), or a pharmaceutically acceptable salt thereof, wherein R⁷ is bromo. In other embodiments are provided compounds of Formula (IId), or a pharmaceutically acceptable salt thereof, wherein R⁷ is halogen in the 2-position. In other embodiments are provided compounds of Formula (IId), wherein R⁷ is fluoro, chloro, or bromo. In other embodiments are provided compounds of Formula (IId), or a pharmaceutically acceptable salt thereof, wherein m is 1 and R⁷ is fluoro. In other embodiments are provided compounds of Formula (IId), or a pharmaceutically acceptable salt thereof, wherein R⁷ is halogen in the 3-position. In other embodiments are provided compounds of Formula (IId), wherein R⁷ is fluoro, chloro, or bromo. In other embodiments are provided compounds of Formula (IId), or a pharmaceutically acceptable salt thereof, wherein m is 1 and R⁷ is fluoro. In other embodiments are provided compounds of Formula (IId), or a pharmaceutically acceptable salt thereof, wherein R⁷ is halogen in the 4-position. In other embodiments are provided compounds of Formula (IId), wherein R⁷ is fluoro, chloro, or bromo. In other embodiments are provided compounds of Formula (IId), or a pharmaceutically acceptable salt thereof, wherein m is 1 and R⁷ is fluoro. In other embodiments are provided compounds of Formula (IId), or a pharmaceutically acceptable salt thereof, wherein R² is N—R¹¹. In other embodiments are provided compounds of Formula (IId), or a pharmaceutically acceptable salt thereof, wherein R¹¹ is hydrogen. In other embodiments are provided compounds of Formula (IId), or a pharmaceutically acceptable salt thereof, wherein R² is O. In other embodiments are provided compounds of Formula (IId), or a pharmaceutically acceptable salt thereof, wherein R² is S. In other embodiments are provided compounds of Formula (IId), or a pharmaceutically acceptable salt thereof, wherein R² is N—R¹¹ and R³ is C—R¹². In other embodiments are provided compounds of Formula (IId), or a pharmaceutically acceptable salt thereof, wherein R¹¹ and R¹² are hydrogen. In other embodiments are provided compounds of Formula (IId), or a pharmaceutically acceptable salt thereof, wherein R² is N—R¹¹ and R³ is N. In other embodiments are provided compounds of Formula (IId), or a pharmaceutically acceptable salt thereof, wherein R¹¹ is hydrogen.

In other embodiments are provided compounds of Formula (IId), or a pharmaceutically acceptable salt thereof, wherein: R¹ is CH or N; R² is NH, O, or S; R⁷ is halogen; R¹⁵ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; m is 1; and n is 0 or 1.

In other embodiments are provided compounds of Formula (IId), or a pharmaceutically acceptable salt thereof, wherein: R¹ is N; R² is NH or 0; and R³ is CH or N.

In other embodiments are provided compounds of Formula (IId), or a pharmaceutically acceptable salt thereof, wherein: R¹ is N; R² is NH; and R³ is CH.

In other embodiments are provided compounds of Formula (IId), or a pharmaceutically acceptable salt thereof, wherein: R¹ is CH; R² is NH; and R³ is CH.

In other embodiments are provided compounds of Formula (IId), or a pharmaceutically acceptable salt thereof, wherein: R¹ is N; R² is NH; and R³ is N.

In other embodiments are provided compounds of Formula (IId), or a pharmaceutically acceptable salt thereof, wherein: R¹ is 0; R² is NH; and R³ is CH.

In other embodiments are provided compounds of Formula (IId), or a pharmaceutically acceptable salt thereof, wherein: R¹ is 0; R² is NH; and R³ is N.

In other embodiments are provided compounds of Formula (IId), or a pharmaceutically acceptable salt thereof, wherein n is 0.

In other embodiments are provided compounds of Formula (IId), or a pharmaceutically acceptable salt thereof, wherein n is 1.

Also provided herein are compounds of Formula (IIe), or a pharmaceutically acceptable salt thereof,

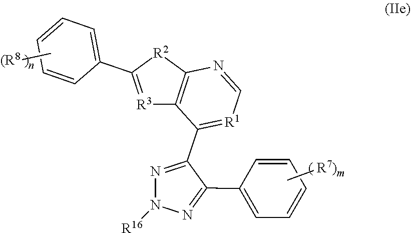

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², and R¹⁶ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5.

In other embodiments are provided compounds of Formula (IIe), or a pharmaceutically acceptable salt thereof, wherein R¹ is N, R² is O or S; and R³ is CH. In other embodiments are provided compounds of Formula (IIe), or a pharmaceutically acceptable salt thereof, wherein R¹ is N, R² is O; and R³ is CH. In other embodiments are provided compounds of Formula (IIe), or a pharmaceutically acceptable salt thereof, wherein R¹ is N, R² is S; and R³ is CH.

In other embodiments are provided compounds of Formula (IIe), or a pharmaceutically acceptable salt thereof, wherein R¹ is C—R⁹. In other embodiments are provided compounds of Formula (IIe), or a pharmaceutically acceptable salt thereof, wherein R¹ is C—H. In other embodiments are provided compounds of Formula (IIe), or a pharmaceutically acceptable salt thereof, wherein R¹ is N. In other embodiments are provided compounds of Formula (IIe), or a pharmaceutically acceptable salt thereof, wherein m is 1, 2, 3, 4, or 5. In other embodiments are provided compounds of Formula (IIe), or a pharmaceutically acceptable salt thereof, wherein m is 1. In other embodiments are provided compounds of Formula (IIe), or a pharmaceutically acceptable salt thereof, wherein R⁷ is halogen. In other embodiments are provided compounds of Formula (IIe), or a pharmaceutically acceptable salt thereof, wherein R⁷ is fluoro, chloro, or bromo. In other embodiments are provided compounds of Formula (IIe), or a pharmaceutically acceptable salt thereof, wherein R⁷ is fluoro. In other embodiments are provided compounds of Formula (IIe), or a pharmaceutically acceptable salt thereof, wherein R⁷ is chloro. In other embodiments are provided compounds of Formula (IIe), or a pharmaceutically acceptable salt thereof, wherein R⁷ is bromo. In other embodiments are provided compounds of Formula (IIe), or a pharmaceutically acceptable salt thereof, wherein R⁷ is halogen in the 2-position. In other embodiments are provided compounds of Formula (IIe), wherein R⁷ is fluoro, chloro, or bromo. In other embodiments are provided compounds of Formula (IIe), or a pharmaceutically acceptable salt thereof, wherein m is 1 and R⁷ is fluoro. In other embodiments are provided compounds of Formula (IIe), or a pharmaceutically acceptable salt thereof, wherein R⁷ is halogen in the 3-position. In other embodiments are provided compounds of Formula (IIe), wherein R⁷ is fluoro, chloro, or bromo. In other embodiments are provided compounds of Formula (IIe), or a pharmaceutically acceptable salt thereof, wherein m is 1 and R⁷ is fluoro. In other embodiments are provided compounds of Formula (IIe), or a pharmaceutically acceptable salt thereof, wherein R⁷ is halogen in the 4-position. In other embodiments are provided compounds of Formula (IIe), wherein R⁷ is fluoro, chloro, or bromo. In other embodiments are provided compounds of Formula (IIe), or a pharmaceutically acceptable salt thereof, wherein m is 1 and R⁷ is fluoro. In other embodiments are provided compounds of Formula (IIe), or a pharmaceutically acceptable salt thereof, wherein R² is N—R¹¹. In other embodiments are provided compounds of Formula (IIe), or a pharmaceutically acceptable salt thereof, wherein R¹¹ is hydrogen. In other embodiments are provided compounds of Formula (IIe), or a pharmaceutically acceptable salt thereof, wherein R² is O. In other embodiments are provided compounds of Formula (IIe), or a pharmaceutically acceptable salt thereof, wherein R² is S. In other embodiments are provided compounds of Formula (IIe), or a pharmaceutically acceptable salt thereof, wherein R² is N—R¹¹ and R³ is C—R¹². In other embodiments are provided compounds of Formula (IIe), or a pharmaceutically acceptable salt thereof, wherein R¹¹ and R¹² are hydrogen. In other embodiments are provided compounds of Formula (IIe), or a pharmaceutically acceptable salt thereof, wherein R² is N—R¹¹ and R³ is N. In other embodiments are provided compounds of Formula (IIe), or a pharmaceutically acceptable salt thereof, wherein R¹¹ is hydrogen.

In other embodiments are provided compounds of Formula (IIe), or a pharmaceutically acceptable salt thereof, wherein: R¹ is CH or N; R² is NH, O, or S; R⁷ is halogen; R¹⁶ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; m is 1; and n is 0 or 1.

In other embodiments are provided compounds of Formula (IIe), or a pharmaceutically acceptable salt thereof, wherein: R¹ is N; R² is NH or O; and R³ is CH or N.

In other embodiments are provided compounds of Formula (IIe), or a pharmaceutically acceptable salt thereof, wherein: R¹ is N; R² is NH; and R³ is CH.

In other embodiments are provided compounds of Formula (IIe), or a pharmaceutically acceptable salt thereof, wherein: R¹ is CH; R² is NH; and R³ is CH.

In other embodiments are provided compounds of Formula (IIe), or a pharmaceutically acceptable salt thereof, wherein: R¹ is N; R² is NH; and R³ is N.

In other embodiments are provided compounds of Formula (IIe), or a pharmaceutically acceptable salt thereof, wherein: R¹ is O; R² is NH; and R³ is CH.

In other embodiments are provided compounds of Formula (IIe), or a pharmaceutically acceptable salt thereof, wherein: R¹ is O; R² is NH; and R³ is N.

In other embodiments are provided compounds of Formula (IIe), or a pharmaceutically acceptable salt thereof, wherein n is 0.

In other embodiments are provided compounds of Formula (IIe), or a pharmaceutically acceptable salt thereof, wherein n is 1.

Also provided herein are compounds of Formula (III), or a pharmaceutically acceptable salt thereof,

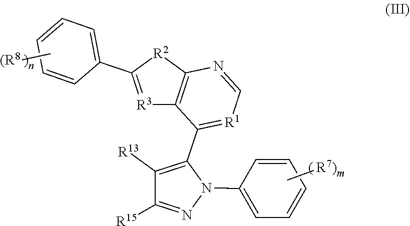

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², R¹³, and R¹¹ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5.

In other embodiments are provided compounds of Formula (III), or a pharmaceutically acceptable salt thereof, wherein R¹ is N, R² is 0 or S; and R³ is CH. In other embodiments are provided compounds of Formula (III), or a pharmaceutically acceptable salt thereof, wherein R¹ is N, R² is 0; and R³ is CH. In other embodiments are provided compounds of Formula (III), or a pharmaceutically acceptable salt thereof, wherein R¹ is N, R² is S; and R³ is CH.

In other embodiments are provided compounds of Formula (III), or a pharmaceutically acceptable salt thereof, wherein R¹ is C—R⁹. In other embodiments are provided compounds of Formula (III), or a pharmaceutically acceptable salt thereof, wherein R¹ is C—H. In other embodiments are provided compounds of Formula (III), or a pharmaceutically acceptable salt thereof, wherein R¹ is N. In other embodiments are provided compounds of Formula (III), or a pharmaceutically acceptable salt thereof, wherein m is 1, 2, 3, 4, or 5. In other embodiments are provided compounds of Formula (III), or a pharmaceutically acceptable salt thereof, wherein m is 1. In other embodiments are provided compounds of Formula (III), or a pharmaceutically acceptable salt thereof, wherein R⁷ is halogen. In other embodiments are provided compounds of Formula (III), or a pharmaceutically acceptable salt thereof, wherein R⁷ is fluoro, chloro, or bromo. In other embodiments are provided compounds of Formula (III), or a pharmaceutically acceptable salt thereof, wherein R⁷ is fluoro. In other embodiments are provided compounds of Formula (III), or a pharmaceutically acceptable salt thereof, wherein R⁷ is chloro. In other embodiments are provided compounds of Formula (III), or a pharmaceutically acceptable salt thereof, wherein R⁷ is bromo. In other embodiments are provided compounds of Formula (III), or a pharmaceutically acceptable salt thereof, wherein R⁷ is halogen in the 2-position. In other embodiments are provided compounds of Formula (III), wherein R⁷ is fluoro, chloro, or bromo. In other embodiments are provided compounds of Formula (III), or a pharmaceutically acceptable salt thereof, wherein m is 1 and R⁷ is fluoro. In other embodiments are provided compounds of Formula (III), or a pharmaceutically acceptable salt thereof, wherein R⁷ is halogen in the 3-position. In other embodiments are provided compounds of Formula (III) wherein R⁷ is fluoro, chloro, or bromo. In other embodiments are provided compounds of Formula (III), or a pharmaceutically acceptable salt thereof, wherein m is 1 and R⁷ is fluoro. In other embodiments are provided compounds of Formula (III), or a pharmaceutically acceptable salt thereof, wherein R⁷ is halogen in the 4-position. In other embodiments are provided compounds of Formula (III), wherein R⁷ is fluoro, chloro, or bromo. In other embodiments are provided compounds of Formula (III), or a pharmaceutically acceptable salt thereof, wherein m is 1 and R⁷ is fluoro. In other embodiments are provided compounds of Formula (III) or a pharmaceutically acceptable salt thereof, wherein R² is N—R¹¹. In other embodiments are provided compounds of Formula (III), or a pharmaceutically acceptable salt thereof, wherein R¹¹ is hydrogen. In other embodiments are provided compounds of Formula (III), or a pharmaceutically acceptable salt thereof, wherein R² is O. In other embodiments are provided compounds of Formula (III), or a pharmaceutically acceptable salt thereof, wherein R² is S. In other embodiments are provided compounds of Formula (III), or a pharmaceutically acceptable salt thereof, wherein R² is N—R¹¹ and R³ is C—R¹². In other embodiments are provided compounds of Formula (III), or a pharmaceutically acceptable salt thereof, wherein R¹¹ and R¹² are hydrogen. In other embodiments are provided compounds of Formula (III), or a pharmaceutically acceptable salt thereof, wherein R² is N—R¹¹ and R³ is N. In other embodiments are provided compounds of Formula (III), or a pharmaceutically acceptable salt thereof, wherein R¹¹ is hydrogen.

In other embodiments are provided compounds of Formula (III), or a pharmaceutically acceptable salt thereof, wherein: R¹ is CH or N; R² is NH, O, or S; R⁷ is halogen; R¹³ is hydrogen; R¹¹ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; m is 1; and n is 0 or 1.

In other embodiments are provided compounds of Formula (III), or a pharmaceutically acceptable salt thereof, wherein: R¹ is N; R² is NH or 0; and R³ is CH or N.

In other embodiments are provided compounds of Formula (III), or a pharmaceutically acceptable salt thereof, wherein: R¹ is N; R² is NH; and R³ is CH.

In other embodiments are provided compounds of Formula (III), or a pharmaceutically acceptable salt thereof, wherein: R¹ is CH; R² is NH; and R³ is CH.

In other embodiments are provided compounds of Formula (III), or a pharmaceutically acceptable salt thereof, wherein: R¹ is N; R² is NH; and R³ is N.

In other embodiments are provided compounds of Formula (III), or a pharmaceutically acceptable salt thereof, wherein: R¹ is 0; R² is NH; and R³ is CH.

In other embodiments are provided compounds of Formula (III), or a pharmaceutically acceptable salt thereof, wherein: R¹ is 0; R² is NH; and R³ is N.

In other embodiments are provided compounds of Formula (III), or a pharmaceutically acceptable salt thereof, wherein n is 0.

In other embodiments are provided compounds of Formula (III), or a pharmaceutically acceptable salt thereof, wherein n is 1.

Also provided herein are compounds of Formula (IV), or a pharmaceutically acceptable salt thereof,

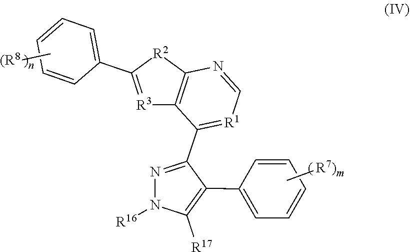

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², R¹⁶, and R¹⁷ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5.

In other embodiments are provided compounds of Formula (IV), or a pharmaceutically acceptable salt thereof, wherein R¹ is N, R² is 0 or S; and R³ is CH. In other embodiments are provided compounds of Formula (IV), or a pharmaceutically acceptable salt thereof, wherein R¹ is N, R² is 0; and R³ is CH. In other embodiments are provided compounds of Formula (IV), or a pharmaceutically acceptable salt thereof, wherein R¹ is N, R² is S; and R³ is CH.

In other embodiments are provided compounds of Formula (IV), or a pharmaceutically acceptable salt thereof, wherein R¹ is C—R⁹. In other embodiments are provided compounds of Formula (IV), or a pharmaceutically acceptable salt thereof, wherein R¹ is C—H. In other embodiments are provided compounds of Formula (IV), or a pharmaceutically acceptable salt thereof, wherein R¹ is N. In other embodiments are provided compounds of Formula (IV), or a pharmaceutically acceptable salt thereof, wherein m is 1, 2, 3, 4, or 5. In other embodiments are provided compounds of Formula (IV), or a pharmaceutically acceptable salt thereof, wherein m is 1. In other embodiments are provided compounds of Formula (IV), or a pharmaceutically acceptable salt thereof, wherein R⁷ is halogen. In other embodiments are provided compounds of Formula (IV), or a pharmaceutically acceptable salt thereof, wherein R⁷ is fluoro, chloro, or bromo. In other embodiments are provided compounds of Formula (IV), or a pharmaceutically acceptable salt thereof, wherein R⁷ is fluoro. In other embodiments are provided compounds of Formula (IV), or a pharmaceutically acceptable salt thereof, wherein R⁷ is chloro. In other embodiments are provided compounds of Formula (IV), or a pharmaceutically acceptable salt thereof, wherein R⁷ is bromo. In other embodiments are provided compounds of Formula (IV), or a pharmaceutically acceptable salt thereof, wherein R⁷ is halogen in the 2-position. In other embodiments are provided compounds of Formula (IV), wherein R⁷ is fluoro, chloro, or bromo. In other embodiments are provided compounds of Formula (IV), or a pharmaceutically acceptable salt thereof, wherein m is 1 and R⁷ is fluoro. In other embodiments are provided compounds of Formula (IV), or a pharmaceutically acceptable salt thereof, wherein R⁷ is halogen in the 3-position. In other embodiments are provided compounds of Formula (IV), wherein R⁷ is fluoro, chloro, or bromo. In other embodiments are provided compounds of Formula (IV), or a pharmaceutically acceptable salt thereof, wherein m is 1 and R⁷ is fluoro. In other embodiments are provided compounds of Formula (IV), or a pharmaceutically acceptable salt thereof, wherein R⁷ is halogen in the 4-position. In other embodiments are provided compounds of Formula (IV), wherein R⁷ is fluoro, chloro, or bromo. In other embodiments are provided compounds of Formula (IV), or a pharmaceutically acceptable salt thereof, wherein m is 1 and R⁷ is fluoro. In other embodiments are provided compounds of Formula (IV), or a pharmaceutically acceptable salt thereof, wherein R² is N—R¹¹. In other embodiments are provided compounds of Formula (IV), or a pharmaceutically acceptable salt thereof, wherein R¹¹ is hydrogen. In other embodiments are provided compounds of Formula (IV), or a pharmaceutically acceptable salt thereof, wherein R² is O. In other embodiments are provided compounds of Formula (IV), or a pharmaceutically acceptable salt thereof, wherein R² is S. In other embodiments are provided compounds of Formula (IV), or a pharmaceutically acceptable salt thereof, wherein R² is N—R¹¹ and R³ is C—R¹². In other embodiments are provided compounds of Formula (IV), or a pharmaceutically acceptable salt thereof, wherein R¹¹ and R¹² are hydrogen. In other embodiments are provided compounds of Formula (IV), or a pharmaceutically acceptable salt thereof, wherein R² is N—R¹¹ and R³ is N. In other embodiments are provided compounds of Formula (IV), or a pharmaceutically acceptable salt thereof, wherein R¹¹ is hydrogen.

In other embodiments are provided compounds of Formula (IV), or a pharmaceutically acceptable salt thereof, wherein: R¹ is CH or N; R² is NH, O, or S; R⁷ is halogen; R¹³ is hydrogen; R¹⁶ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; R¹⁷ is hydrogen; m is 1; and n is 0 or 1.

In other embodiments are provided compounds of Formula (IV), or a pharmaceutically acceptable salt thereof, wherein: R¹ is N; R² is NH or O; and R³ is CH or N.

In other embodiments are provided compounds of Formula (IV), or a pharmaceutically acceptable salt thereof, wherein: R¹ is N; R² is NH; and R³ is CH.

In other embodiments are provided compounds of Formula (IV), or a pharmaceutically acceptable salt thereof, wherein: R¹ is CH; R² is NH; and R³ is CH.

In other embodiments are provided compounds of Formula (IV), or a pharmaceutically acceptable salt thereof, wherein: R¹ is N; R² is NH; and R³ is N.

In other embodiments are provided compounds of Formula (IV), or a pharmaceutically acceptable salt thereof, wherein: R¹ is O; R² is NH; and R³ is CH.

In other embodiments are provided compounds of Formula (IV), or a pharmaceutically acceptable salt thereof, wherein: R¹ is O; R² is NH; and R³ is N.

In other embodiments are provided compounds of Formula (IV), or a pharmaceutically acceptable salt thereof, wherein n is 0.

In other embodiments are provided compounds of Formula (IV), or a pharmaceutically acceptable salt thereof, wherein n is 1.

Also provided herein are compounds of Formula (V), or a pharmaceutically acceptable salt thereof,

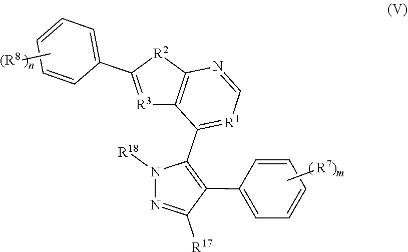

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², R⁷, and R¹⁸ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5.

In other embodiments are provided compounds of Formula (V), or a pharmaceutically acceptable salt thereof, wherein R¹ is N, R² is 0 or S; and R³ is CH. In other embodiments are provided compounds of Formula (V), or a pharmaceutically acceptable salt thereof, wherein R¹ is N, R² is 0; and R³ is CH. In other embodiments are provided compounds of Formula (V), or a pharmaceutically acceptable salt thereof, wherein R¹ is N, R² is S; and R³ is CH.

In other embodiments are provided compounds of Formula (V), or a pharmaceutically acceptable salt thereof, wherein R¹ is C—R⁹. In other embodiments are provided compounds of Formula (V), or a pharmaceutically acceptable salt thereof, wherein R¹ is C—H. In other embodiments are provided compounds of Formula (V), or a pharmaceutically acceptable salt thereof, wherein R¹ is N. In other embodiments are provided compounds of Formula (V), or a pharmaceutically acceptable salt thereof, wherein m is 1, 2, 3, 4, or 5. In other embodiments are provided compounds of Formula (V), or a pharmaceutically acceptable salt thereof, wherein m is 1. In other embodiments are provided compounds of Formula (V), or a pharmaceutically acceptable salt thereof, wherein R⁷ is halogen. In other embodiments are provided compounds of Formula (V), or a pharmaceutically acceptable salt thereof, wherein R⁷ is fluoro, chloro, or bromo. In other embodiments are provided compounds of Formula (V), or a pharmaceutically acceptable salt thereof, wherein R⁷ is fluoro. In other embodiments are provided compounds of Formula (V), or a pharmaceutically acceptable salt thereof, wherein R⁷ is chloro. In other embodiments are provided compounds of Formula (V), or a pharmaceutically acceptable salt thereof, wherein R⁷ is bromo. In other embodiments are provided compounds of Formula (V), or a pharmaceutically acceptable salt thereof, wherein R⁷ is halogen in the 2-position. In other embodiments are provided compounds of Formula (V), wherein R⁷ is fluoro, chloro, or bromo. In other embodiments are provided compounds of Formula (V), or a pharmaceutically acceptable salt thereof, wherein m is 1 and R⁷ is fluoro. In other embodiments are provided compounds of Formula (V), or a pharmaceutically acceptable salt thereof, wherein R⁷ is halogen in the 3-position. In other embodiments are provided compounds of Formula (V), wherein R⁷ is fluoro, chloro, or bromo. In other embodiments are provided compounds of Formula (V), or a pharmaceutically acceptable salt thereof, wherein m is 1 and R⁷ is fluoro. In other embodiments are provided compounds of Formula (V), or a pharmaceutically acceptable salt thereof, wherein R⁷ is halogen in the 4-position. In other embodiments are provided compounds of Formula (V), wherein R⁷ is fluoro, chloro, or bromo. In other embodiments are provided compounds of Formula (V), or a pharmaceutically acceptable salt thereof, wherein m is 1 and R⁷ is fluoro. In other embodiments are provided compounds of Formula (V), or a pharmaceutically acceptable salt thereof, wherein R² is N—R¹¹. In other embodiments are provided compounds of Formula (V), or a pharmaceutically acceptable salt thereof, wherein R¹¹ is hydrogen. In other embodiments are provided compounds of Formula (V), or a pharmaceutically acceptable salt thereof, wherein R² is O. In other embodiments are provided compounds of Formula (V), or a pharmaceutically acceptable salt thereof, wherein R² is S. In other embodiments are provided compounds of Formula (V), or a pharmaceutically acceptable salt thereof, wherein R² is N—R¹¹ and R³ is C—R¹². In other embodiments are provided compounds of Formula (V), or a pharmaceutically acceptable salt thereof, wherein R¹¹ and R¹² are hydrogen. In other embodiments are provided compounds of Formula (V), or a pharmaceutically acceptable salt thereof, wherein R² is N—R¹¹ and R³ is N. In other embodiments are provided compounds of Formula (V), or a pharmaceutically acceptable salt thereof, wherein R¹¹ is hydrogen.

In other embodiments are provided compounds of Formula (V), or a pharmaceutically acceptable salt thereof, wherein: R¹ is CH or N; R² is NH, O, or S; R⁷ is halogen; R¹³ is hydrogen; R¹⁷ is hydrogen; R¹⁸ is C₁-C₆alkyl, C₁-C₆haloalkyl, —OC₁-C₆haloalkyl, C₁-C₆deuteroalkyl, C₁-C₆hydroxyalkyl, or C₁-C₆aminoalkyl, wherein each alkyl is optionally and independently substituted with one or more R^{1b}; m is 1; and n is 0 or 1.

In other embodiments are provided compounds of Formula (V), or a pharmaceutically acceptable salt thereof, wherein: R¹ is N; R² is NH or O; and R³ is CH or N.

In other embodiments are provided compounds of Formula (V), or a pharmaceutically acceptable salt thereof, wherein: R¹ is N; R² is NH; and R³ is CH.

In other embodiments are provided compounds of Formula (V), or a pharmaceutically acceptable salt thereof, wherein: R¹ is CH; R² is NH; and R³ is CH.

In other embodiments are provided compounds of Formula (V), or a pharmaceutically acceptable salt thereof, wherein: R¹ is N; R² is NH; and R³ is N.

In other embodiments are provided compounds of Formula (V), or a pharmaceutically acceptable salt thereof, wherein: R¹ is O; R² is NH; and R³ is CH.

In other embodiments are provided compounds of Formula (V), or a pharmaceutically acceptable salt thereof, wherein: R¹ is O; R² is NH; and R³ is N.

In other embodiments are provided compounds of Formula (V), or a pharmaceutically acceptable salt thereof, wherein n is 0.

In other embodiments are provided compounds of Formula (V), or a pharmaceutically acceptable salt thereof, wherein n is 1.

Also provided herein are compounds according to Formula (VI), or a pharmaceutically acceptable salt thereof,

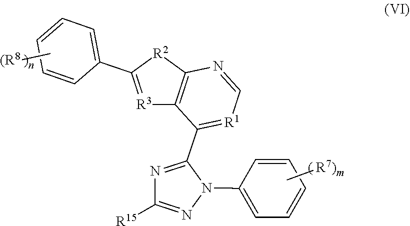

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², and R¹¹ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5.

In other embodiments are provided compounds of Formula (VI), or a pharmaceutically acceptable salt thereof, wherein R¹ is N, R² is O or S; and R³ is CH. In other embodiments are provided compounds of Formula (VI), or a pharmaceutically acceptable salt thereof, wherein R¹ is N, R² is O; and R³ is CH. In other embodiments are provided compounds of Formula (VI), or a pharmaceutically acceptable salt thereof, wherein R¹ is N, R² is S; and R³ is CH.

Also provided herein are compounds of Formula (VII), or a pharmaceutically acceptable salt thereof,

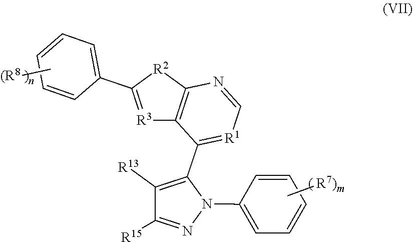

wherein:
R¹ is C—R⁹ or N;
R² is N—R¹¹, O, or S;
R³ is C—R¹² or N;
R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², R¹³, and R¹¹ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5.

In other embodiments are provided compounds of Formula (VII), or a pharmaceutically acceptable salt thereof, wherein R¹ is N, R² is O or S; and R³ is CH. In other embodiments are provided compounds of Formula (VII), or a pharmaceutically acceptable salt thereof, wherein R¹ is N, R² is O; and R³ is CH. In other embodiments are provided compounds of Formula (VII), or a pharmaceutically acceptable salt thereof, wherein R¹ is N, R² is S; and R³ is CH.

Also provided herein are compounds according to Formula (VIII),

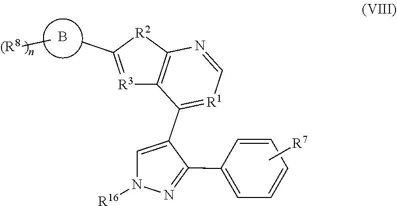

wherein:
B is C₂-C₆alkynyl, C₆-C₁₀aryl, heteroaryl, heterocycloalkyl, or C₃-C₁₂ cycloalkyl;
R¹ is C—R⁹ or N;
R² is N—R¹¹, O, or S;
R³ is C—R¹² or N;
R⁷ is halogen;
R⁸ are independently selected from deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², and R¹⁶ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
n is 0, 1, 2, 3, 4, or 5; or a pharmaceutically acceptable salt thereof.

In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), or a pharmaceutically acceptable salt thereof, wherein B is C₂-C₆alkynyl and R⁷ is halogen. In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), wherein B is C₂-C₆alkynyl and R⁷ is fluoro, or a pharmaceutically acceptable salt thereof.

In other embodiments are provided compounds of Formula (VIII), wherein B is C₂-C₆alkynyl and R⁷ is fluoro, or a pharmaceutically acceptable salt thereof. In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), wherein B is C₆-C₁₀aryl and R⁷ is halogen, or a pharmaceutically acceptable salt thereof. In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), wherein B is C₆-C₁₀aryl and R⁷ is fluoro, or a pharmaceutically acceptable salt thereof.

In other embodiments are provided compounds of Formula (VIII), wherein B is C₆-C₁₀aryl and R⁷ is fluoro, or a pharmaceutically acceptable salt thereof. In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), wherein B is heterocycloalkyl and R⁷ is halogen, or a pharmaceutically acceptable salt thereof. In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), wherein B is heterocycloalkyl and R⁷ is halogen, or a pharmaceutically acceptable salt thereof. In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), wherein B is heterocycloalkyl and R⁷ is fluoro, or a pharmaceutically acceptable salt thereof.

In other embodiments are provided compounds of Formula (VIII), wherein B is heterocycloalkyl and R⁷ is fluoro, or a pharmaceutically acceptable salt thereof. In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), wherein B is heteroaryl and R⁷ is halogen, or a pharmaceutically acceptable salt thereof. In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), wherein B is heteroaryl and R⁷ is fluoro, or a pharmaceutically acceptable salt thereof. In other embodiments are provided compounds of Formula (VIII), or a pharmaceutically acceptable salt thereof, wherein R¹ is N, R² is O or S; and R³ is CH. In other embodiments are provided compounds of Formula (VIII), or a pharmaceutically acceptable salt thereof, wherein R¹ is N, R² is O; and R³ is CH. In other embodiments are provided compounds of Formula (VIII), or a pharmaceutically acceptable salt thereof, wherein R¹ is N, R² is S; and R³ is CH.

In other embodiments are provided compounds of Formula (VIII), wherein B is C₆-C₁₀aryl; R¹ is N; R² is N—R¹¹; R³ is N; R⁷ is fluoro; and R¹¹ is hydrogen; or a pharmaceutically acceptable salt thereof.

In other embodiments are provided compounds of Formula (VIII), wherein B is heteroaryl; R¹ is N; R² is N—R¹¹; R³ is N; R⁷ is fluoro; and R¹¹ is hydrogen; or a pharmaceutically acceptable salt thereof.

In other embodiments are provided compounds of Formula (VIII), wherein B is heterocycloalkyl; R¹ is N; R² is N—R¹¹; R³ is N; R⁷ is fluoro; and R¹¹ is hydrogen; or a pharmaceutically acceptable salt thereof.

Also provided herein are compounds of Formula (VIIIa),

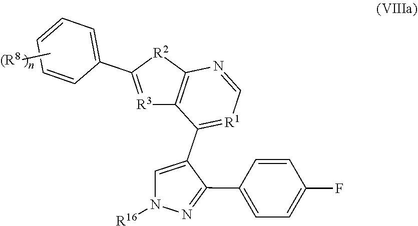

wherein:
R¹ is C—R⁹ or N;
R² is N—R¹¹, O, or S;
R³ is C—R¹² or N;
R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R⁹, R¹¹, R¹², and R¹⁶ are each independently selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
n is 0, 1, 2, 3, 4, or 5; or a pharmaceutically acceptable salt thereof.

In other embodiments are provided compounds of Formula (VIIIa), or a pharmaceutically acceptable salt thereof, wherein R¹ is N, R² is O or S; and R³ is CH. In other embodiments are provided compounds of Formula (VIIIa), or a pharmaceutically acceptable salt thereof, wherein R¹ is N, R² is O; and R³ is CH. In other embodiments are provided compounds of Formula (VIIIa), or a pharmaceutically acceptable salt thereof, wherein R¹ is N, R² is S; and R³ is CH

Also provided herein are compounds of Formula (IX),

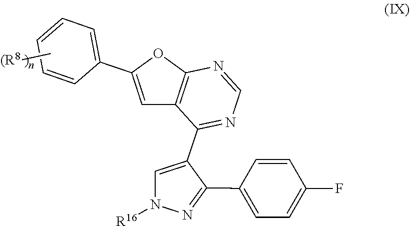

wherein:
each R⁸ is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R¹⁶ is selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
n is 0, 1, 2, 3, 4, or 5; or a pharmaceutically acceptable salt thereof.

In other embodiments are provided compounds of Formula (IX), wherein n is 0, or a pharmaceutically acceptable salt thereof.

Also provided herein are compounds of Formula (X),

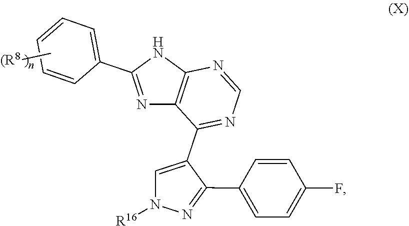

wherein:
each R⁸ is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R¹⁶ is selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
n is 0, 1, 2, 3, 4, or 5; or a pharmaceutically acceptable salt thereof.

In other embodiments are provided compounds of Formula (X), wherein n is 0, or a pharmaceutically acceptable salt thereof.

Also provided herein are compounds of Formula (XI),

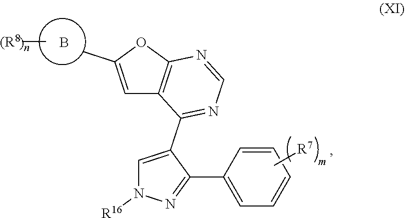

wherein:
B is aryl, heteroaryl, heterocycloalkyl, or C₃-C₁₂ cycloalkyl;
each R⁷ and R⁸ are independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R¹⁶ is selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
m is 0, 1, 2, 3, 4, or 5; and
n is 0, 1, 2, 3, 4, or 5; or a pharmaceutically acceptable salt thereof.

In other embodiments are provided compounds of Formula (XI), or a pharmaceutically acceptable salt thereof, wherein m is 1, 2, 3, 4, or 5. In other embodiments are provided compounds of Formula (XI), or a pharmaceutically acceptable salt thereof, wherein m is 1. In other embodiments are provided compounds of Formula (XI), or a pharmaceutically acceptable salt thereof, wherein R⁷ is halogen. In other embodiments are provided compounds of Formula (XI), or a pharmaceutically acceptable salt thereof, wherein R⁷ is fluoro, chloro, or bromo. In other embodiments are provided compounds of Formula (XI), or a pharmaceutically acceptable salt thereof, wherein R⁷ is fluoro. In other embodiments are provided compounds of Formula (XI), or a pharmaceutically acceptable salt thereof, wherein R⁷ is chloro. In other embodiments are provided compounds of Formula (XI), or a pharmaceutically acceptable salt thereof, wherein R⁷ is bromo. In other embodiments are provided compounds of Formula (XI), or a pharmaceutically acceptable salt thereof, wherein R⁷ is halogen in the 2-position. In other embodiments are provided compounds of Formula (XI), wherein R⁷ is fluoro, chloro, or bromo. In other embodiments are provided compounds of Formula (XI), or a pharmaceutically acceptable salt thereof, wherein m is 1 and R⁷ is fluoro. In other embodiments are provided compounds of Formula (XI), or a pharmaceutically acceptable salt thereof, wherein R⁷ is halogen in the 3-position. In other embodiments are provided compounds of Formula (XI), wherein R⁷ is fluoro, chloro, or bromo. In other embodiments are provided compounds of Formula (XI), or a pharmaceutically acceptable salt thereof, wherein m is 1 and R⁷ is fluoro. In other embodiments are provided compounds of Formula (XI), or a pharmaceutically acceptable salt thereof, wherein R⁷ is halogen in the 4-position. In other embodiments are provided compounds of Formula (XI), wherein R⁷ is fluoro, chloro, or bromo. In other embodiments are provided compounds of Formula (XI), or a pharmaceutically acceptable salt thereof, wherein m is 1 and R⁷ is fluoro. In other embodiments are provided compounds of Formula (XI), or a pharmaceutically acceptable salt thereof, wherein B is aryl. In other embodiments are provided compounds of Formula (XI), or a pharmaceutically acceptable salt thereof, wherein B is heteroaryl. In other embodiments are provided compounds of Formula (XI), or a pharmaceutically acceptable salt thereof, wherein B is heterocycloalkyl. In other embodiments are provided compounds of Formula (XI), or a pharmaceutically acceptable salt thereof, wherein B is C₃-C₁₂ cycloalkyl.

Also provided herein are compounds of Formula (XII),

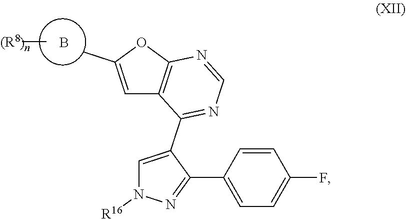

wherein:
B is aryl, heteroaryl, heterocycloalkyl, or C₃-C₁₂ cycloalkyl;
each R⁸ is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R¹⁶ is selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C₆-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
n is 0, 1, 2, 3, 4, or 5; or a pharmaceutically acceptable salt thereof.

In other embodiments are provided compounds of Formula (XII), or a pharmaceutically acceptable salt thereof, wherein B is aryl. In other embodiments are provided compounds of Formula (XII), or a pharmaceutically acceptable salt thereof, wherein B is heteroaryl. In other embodiments are provided compounds of Formula (XII), or a pharmaceutically acceptable salt thereof, wherein B is heterocycloalkyl. In other embodiments are provided compounds of Formula (XII), or a pharmaceutically acceptable salt thereof, wherein B is C₃-C₁₂ cycloalkyl. In other embodiments are provided compounds of Formula (XII), or a pharmaceutically acceptable salt thereof, wherein n is 1, 2, or 3. In other embodiments are provided compounds of Formula (XII), or a pharmaceutically acceptable salt thereof, wherein n is 1. In other embodiments are provided compounds of Formula (XII), or a pharmaceutically acceptable salt thereof, wherein n is 2. In other embodiments are provided compounds of Formula (XII), or a pharmaceutically acceptable salt thereof, wherein n is 3.

In other embodiments are provided compounds of Formula (XII), or a pharmaceutically acceptable salt thereof, wherein B is pyridinyl, pyrazolyl, pyrimidinyl, pyrazinyl, pyridazinyl, imidazolyl, thiazolyl, isothiazolyl, oxazolyl, isoxazolyl, oxadiazolyl, triazolyl, thiadiazolyl, or tetrazolyl. In some embodiments, B is pyridinyl. In some embodiments, B is pyrazolyl. In some embodiments, B is pyrimidinyl. In some embodiments, B is pyrazinyl. In some embodiments, B is pyridazinyl. In some embodiments, B is imidazolyl. In some embodiments, B is thiazolyl. In some embodiments, B is isothiazolyl. In some embodiments, B is oxazolyl. In some embodiments, B is isoxazolyl. In some embodiments, B is oxadiazolyl. In some embodiments, B is triazolyl. In some embodiments, B is thiadiazolyl. In some embodiments, B is tetrazolyl.

In other embodiments are provided compounds of Formula (XII), or a pharmaceutically acceptable salt thereof, wherein B is heterocycloalkyl. In some embodiments, B is 3,6-dihydropyridinyl, piperazinyl, tetrahydropyridinyl, piperidinyl, tetrahydrofuranyl, dihydropyranyl, tetrahydropyranyl, dioxalanyl, or 2-azaspiro[3.4]octan-2-yl. In other embodiments are provided compounds of Formula (XII), or a pharmaceutically acceptable salt thereof, wherein R¹⁶ is hydrogen, ethyl, 1,1-dioxo-1,6-thietan-3-yl, 4-butanamidyl, 1-methylpyrrolidin-2-onyl, 3-fluorocyclobutyl, 3-fluorooxan-4-yl, 3-fluoropropyl, 3,3-difluoropropyl, 2,2-dimethylpropanamidinyl, 1-imino-1,6-thiolan-1-onyl, 1-methylcyclobutan-1-ol, 3,3-difluorocyclobutyl, 1,6-thiolane-1,1-dionyl, 1-(3-fluorooxan-4-yl), 1-(methanesulfonyl)-5-methylpyrrolidin-3-yl, 2-hydroxy-2-methylpropyl, 2-fluorocyclohexyl, 3-(methanesulfonyl)propyl, 4,4-dimethyloxetan-2-yl)methyl, 5,5-dimethyloxolan-3-yl, 1-[2-(difluoromethoxy)]ethyl, 3-cyanopropyl, or 3-(methanesulfonyl)cyclobutyl.

In other embodiments are provided compounds of Formula (XII), or a pharmaceutically acceptable salt thereof, wherein R¹⁶ is hydrogen, ethyl, 1,1-dioxo-1λ6-thietan-3-yl, 4-butanamidyl, 1-methylpyrrolidin-2-onyl, 3-fluorocyclobutyl, 3-fluorooxan-4-yl, 3-fluoropropyl, 3,3-difluoropropyl, 2,2-dimethylpropanamidinyl, 1-imino-1,6-thiolan-1-onyl, 1-methylcyclobutan-1-ol, 3,3-difluorocyclobutyl, 1,6-thiolane-1,1-dionyl, 1-(3-fluorooxan-4-yl), 1-(methanesulfonyl)-5-methylpyrrolidin-3-yl, 2-hydroxy-2-methylpropyl, 2-fluorocyclohexyl, 3-(methanesulfonyl)propyl, 4,4-dimethyloxetan-2-yl)methyl, 5,5-dimethyloxolan-3-yl, 1-[2-(difluoromethoxy)]ethyl, 3-cyanopropyl, 3-(methanesulfonyl)cyclobutyl, cyclobutan-1-ol, cyclobutane-1-carbonitrile, 2,2-difluoropropan-1-ol, or 2,2-difluorocyclopropyl.

Also provided herein are methods of treating cancer in a subject, wherein the cancer has been determined to comprise a C797S mutation in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

Also provided herein are methods of treating metastatic cancer in a subject, wherein the metastatic cancer has been determined to comprise a C797S mutation in the epidermal growth factor receptor (EGFR) protein, comprising administering to the subject a composition comprising an effective amount of a compound Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments, the cancer is a locally advanced cancer.

In some embodiments, the cancer is an unresectable cancer. In some embodiments, the cancer is a resectable cancer. In some embodiments, the cancer is a metastatic cancer. In some embodiments, the cancer has been further determined to comprise an exon 19 deletion mutation in the epidermal growth factor receptor (EGFR) protein. In some embodiments, the cancer has been further determined to comprise an L858R mutation in the epidermal growth factor receptor (EGFR) protein. In some embodiments, the cancer has been further determined not to comprise a T790M mutation in the epidermal growth factor receptor (EGFR) protein. In some embodiments, the cancer has been determined to comprise L858R and C797S mutations in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments, the metastatic cancer has been determined to comprise exon 19 deletion and C797S mutations in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

In some embodiments, the cancer has been further determined not to comprise a T790M mutation in the epidermal growth factor receptor (EGFR) protein.

In some embodiments, the cancer is selected from the group consisting of breast cancer, colon cancer, lung cancer, non-small cell lung cancer, gastric cancer, prostate cancer, and colorectal cancer. In other embodiments, the cancer comprises one or more central nervous system (CNS) metastases (e.g., brain metastases).

In some embodiments, the subject has received one or more prior therapy for treatment of the cancer prior to administration to the subject of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

In some embodiments, the subject has received one or more prior therapy for treatment of the cancer prior to administration to the subject of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, wherein the one more prior therapy exhibits inhibitory activity against cancer comprising a T790M mutation in the epidermal growth factor receptor (EGFR) protein. In some embodiments, the one more prior therapy exhibits inhibitory activity against cancer comprising a T790M mutation in the epidermal growth factor receptor (EGFR) protein is osimertinib.

In some embodiments, the subject has been administered osimertinib prior to administration to the subject of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

In some embodiments, are provided methods of treating a tumor in a subject, wherein the tumor has been determined to comprise a C797S mutation in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

Also provided herein are methods of treating a tumor in a subject following resection of the tumor, wherein the tumor has been determined to comprise a C797S mutation in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

Further provided herein are methods of treating a metastatic tumor in a subject, wherein the metastatic tumor has been determined to comprise a C797S mutation in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments, the tumor is a locally advanced tumor. In some embodiments, the tumor is an unresectable tumor. In some embodiments, the tumor is a resectable tumor. In some embodiments, the tumor has been further determined not to comprise a T790M mutation in the epidermal growth factor receptor (EGFR) protein. In some embodiments, the tumor is derived from a cancer selected from the group consisting of breast cancer, colon cancer, lung cancer, gastric cancer, prostate cancer, and colorectal cancer. In some embodiments, the tumor comprises one or more central nervous system (CNS) tumors. In some embodiments, the subject has received one or more prior therapy for treatment of the tumor prior to administration to the subject of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments, the subject has received one or more prior therapy for treatment of the tumor prior to administration to the subject of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, wherein the one or more prior therapy that exhibits inhibitory activity against cancer comprising a T790M mutation in the epidermal growth factor receptor (EGFR) protein. In some embodiments, the one more prior therapy exhibits inhibitory activity against cancer comprising a T790M mutation in the epidermal growth factor receptor (EGFR) protein is osimertinib.

Also provided herein are methods of treating cancer in a subject, wherein the cancer has progressed following administration to the subject of one or more prior therapy that exhibits inhibitory activity against cancer comprising a T790M mutation in the epidermal growth factor receptor (EGFR) protein, and wherein the cancer been determined to comprise a C797S mutation in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

Also provided herein are methods of treating cancer in a subject, wherein the cancer has progressed following administration to the subject of osimertinib, and wherein the cancer been determined to comprise a C797S mutation in the epidermal growth factor receptor (EGFR) protein, comprising administering to the subject a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments, the cancer comprises one or more central nervous system (CNS) metastases. In some embodiments, the cancer is lung cancer. In some embodiments, the lung cancer is non-small cell lung cancer.

Also provided herein are any of the methods described herein, wherein the composition is administered to the subject in need thereof orally, parentally, intravenously, subcutaneously, or intracerebrally.

Further provided herein are any of the methods described further comprising administering to the subject in need thereof one or more additional anticancer agents.

In some embodiments, the one or more additional anticancer agents comprises one or more agents selected from HER2 inhibitors, HER2-CD3 bispecific antibodies, HER2-immune targeting bispecific antibodies, anti-HER2 chimeric antigen receptor (CAR) T cells, anti-HER2 chimeric antigen receptor (CAR) cytotoxic T-lymphocytes (CTLs), anti-HER2 chimeric antigen receptor (CAR) natural killer (NK) cells, anti-HER2 chimeric antigen receptor (CAR) cytokine-induced killer (CIK) cells, epidermal growth factor receptor (EGFR) inhibitors, poly-ADP-ribose polymerase (PARP) inhibitors, PD-1 inhibitors, PD-L1 inhibitors, Phosphoinositide 3-kinase (PI3K) inhibitors, and chemotherapeutic agents. In other embodiments, the one or more additional cancer agents are selected from the group consisting of erlotinib, gefitinib, afatanib, trastuzumab, trastuzumab and hyaluronidase, capecitabine, trastuzumab and capecitabine, tucatinib, lapatinib, neratinib, dacomitinib, pertuzumab, margetuximab, trastuzumab emtansine, trastuzumab deruxtecan, ZW49 (Zymeworks), A166 (Klaus Pharma), ARX788 (Ambrx), RC48-ADC (RemeGen), vic-trastuzumab duocarmazine, zanidatamab (ZW25), zenocutuzamab (MCLA-128), ISB 1302, poziotinib, pyrotinib, mobocertinib (TAK-788), BDTX-189, osimertinib, cetuximab, panitumumab, necitumumab, vandetanib, brigatinib, icotinib, niraparib, olaparib, talazoparib, rucaparib, veliparib, iniparib, pamiparib (BGB-290), CEP-9722, E7016, pembrolizumab, nivolumab, cemiplimab, JTX-4014, spartalizumab (PDR001), camrelizumab (SHR1210), sintilimab (1B1308), tislelizumab (BGB-A317), toripalimab (JS 001), dostarlimab (TSR-042, WBP-285), INCMGA00012 (MGA012), AMP-224, AMP-514, atezolizumab, avelumab, durvalumab, KN035, CK-301, AUNP12, CA-170, and BMS-986189. In some embodiments, the one or more additional cancer agents are selected from osimertinib, erlotinib, gefitinib, and (d) afatinib. In some embodiments, the one or more additional cancer agents is osimertinib. In some embodiments, the one or more additional cancer agents is erlotinib. In some embodiments, the one or more additional cancer agents is gefitinib. In some embodiments, the one or more additional cancer agents is afatinib.

Also provided herein are any of the disclosed methods, wherein the method further comprises treating the subject in need thereof with radiation.

Also provided herein are any of the disclosed methods wherein the subject is a human.

Further provided herein are uses of a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, for manufacture of a medicament for treatment of a subject having cancer, wherein the cancer comprises a C797S mutation in the epidermal growth factor receptor (EGFR) protein.

Also provided herein are uses of a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, for manufacture of a medicament for the treatment of cancer in a subject, wherein the cancer comprises (a) one or more central nervous system (CNS) metastases, and (b) a C797S mutation in the epidermal growth factor receptor (EGFR) protein.

Further provided herein are kits comprising (i) a composition comprising an effective amount of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, and (ii) instructions for using the kit in treating cancer in a human, wherein the cancer comprises a C797S mutation in the epidermal growth factor receptor (EGFR) protein. Further provided herein are such kits, wherein the kits further comprises one or more additional anticancer agents. In some embodiments, the one or more additional anticancer agents comprises one or more agents selected from HER2 inhibitors, HER2-CD3 bispecific antibodies, HER2-immune targeting bispecific antibodies, anti-HER2 chimeric antigen receptor (CAR) T cells, anti-HER2 chimeric antigen receptor (CAR) cytotoxic T-lymphocytes (CTLs), anti-HER2 chimeric antigen receptor (CAR) natural killer (NK) cells, anti-HER2 chimeric antigen receptor (CAR) cytokine-induced killer (CIK) cells, epidermal growth factor receptor (EGFR) inhibitors, poly-ADP-ribose polymerase (PARP) inhibitors, PD-1 inhibitors, PD-L1 inhibitors, Phosphoinositide 3-kinase (PI3K) inhibitors, and chemotherapeutic agents. In other embodiments, the one or more additional cancer agents are selected from the group consisting of erlotinib, gefitinib, afatanib, trastuzumab, trastuzumab and hyaluronidase, capecitabine, trastuzumab and capecitabine, tucatinib, lapatinib, neratinib, dacomitinib, pertuzumab, margetuximab, trastuzumab emtansine, trastuzumab deruxtecan, ZW49 (Zymeworks), A166 (Klaus Pharma), ARX788 (Ambrx), RC48-ADC (RemeGen), vic-trastuzumab duocarmazine, zanidatamab (ZW25), zenocutuzamab (MCLA-128), ISB 1302, poziotinib, pyrotinib, mobocertinib (TAK-788), and BDTX-189, osimertinib, cetuximab, panitumumab, necitumumab, vandetanib, brigatinib, icotinib, niraparib, olaparib, talazoparib, rucaparib, veliparib, iniparib, pamiparib (BGB-290), CEP-9722, E7016, pembrolizumab, nivolumab, cemiplimab, JTX-4014, spartalizumab (PDR001), camrelizumab (SHR1210), sintilimab (IBI308), tislelizumab (BGB-A317), toripalimab (JS 001), dostarlimab (TSR-042, WBP-285), INCMGA00012 (MGA012), AMP-224, AMP-514, atezolizumab, avelumab, durvalumab, KN035, CK-301, AUNP12, CA-170, and BMS-986189.

As used in the specification and appended claims, unless specified to the contrary, the following terms have the meaning indicated below.

As used herein and in the appended claims, the singular forms “a,” “an,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub-combinations of ranges and specific embodiments therein are intended to be included.

The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Administering” when used in conjunction with a therapeutic means to administer a therapeutic systemically or locally, as directly into or onto a target tissue, or to administer a therapeutic to a subject whereby the therapeutic positively impacts the tissue to which it is targeted. Thus, as used herein, the term “administering”, when used in conjunction with a composition described herein, can include, but is not limited to, providing a composition into or onto the target tissue; providing a composition systemically to a subject by, e.g., oral administration whereby the therapeutic reaches the target tissue or cells. “Administering” a composition may be accomplished by injection, topical administration, and oral administration or by other methods alone or in combination with other known techniques.

The term “C₂-C₆alkenyl” as used herein, means an alkyl moiety comprising 2 to 6 carbon atoms having at least one carbon-carbon double bond. The carbon-carbon double bond in such a group may be anywhere along the 2 to 6 carbon atom chain that will result in a stable compound. Examples of such groups include, but are not limited to, ethenyl, propenyl, butenyl, allyl, and pentenyl. The alkenyl may be in either the cis or trans conformation about the double bond(s), and should be understood to include both isomers.

Examples of alkenyls include, but are not limited to ethenyl (—CH═CH₂), 1-propenyl (—CH₂CH═CH₂), isopropenyl [—C(CH₃)=CH₂], butenyl, 1,3-butadienyl and the like. Whenever it appears herein, a numerical range such as “C₂-C₆ alkenyl” means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₈ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl.

The term “C₁-C₆alkyl,” as used herein, refers to a straight or branched chain hydrocarbon monoradical, which may be fully saturated or unsaturated, having from one to about ten carbon atoms, or from one to six carbon atoms. Examples of saturated hydrocarbon monoradical include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyaminol, isobutyl, sec-butyl, t-butyl, n-pentyl, isopentyl, neopentyl, tert-amyl and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “C₂-C₆alkynyl,” as used herein, means an alkyl moiety comprising from 2 to 6 carbon atoms and having at least one carbon-carbon triple bond. The carbon-carbon triple bond in such a group may be anywhere along the 2 to 6 carbon chain that will result in a stable compound. Examples of such groups include, but are not limited to, ethyne, propyne, 1-butyne, 2-butyne, 1-pentyne, 2-pentyne, 1-hexyne, 2-hexyne, and 3-hexyne, ethynyl, 2-propynyl, 2-butynyl, 1,3-butadiynyl and the like. Whenever it appears herein, a numerical range such as “C₂-C₆ alkynyl” means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated.

The term “C₆-C₁₀aryl,” as used herein, refers to a radical derived from a hydrocarbon ring system comprising hydrogen, 6 to 10 carbon atoms and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl.

The term “C₁-C₆ aminoalkyl,” as used herein, refers to a C₁-C₆alkyl radical, as defined above, that is substituted with one or more amino groups. The amino groups in such C₁-C₆ aminoalkyl groups may be unsubstituted, mono-substituted, or disubstituted. Examples of C₁-C₆ aminoalkyl groups include, but are not limited to, —CH₂NH₂, —CH₂N(H)CH₃, —CH₂N(CH₃)₂, and the like.

The term “C₃-C₁₂ cycloalkyl” refers to a partially or fully saturated, monocyclic, or polycyclic carbocyclic ring comprising from 3 to 12 carbon atoms, which may include fused (when fused with an aryl or a heteroaryl ring, the cycloalkyl is bonded through a non-aromatic ring atom) or bridged ring systems. Representative cycloalkyls include. In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethylbicyclo[2.2.1]heptanyl. Partially saturated cycloalkyls include, for example cyclopentenyl, cyclohexenyl, cycloheptenyl, and cyclooctenyl

The term “C₁-C₆deuteroalkyl,” as used herein, means a C₁-C₆alkyl group as defined herein wherein one or more hydrogen atoms in the C₁-C₆alkyl group is replaced with a deuterium atom.

The term “C₁-C₆ haloalkyl,” as used herein, refers to a C₁-C₆alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo2-fluoropropyl, 1,2-dibromoethyl, and the like.

The term “C₁-C₆ hydroxyalkyl,” as used herein, refers to a C₁-C₆alkyl radical, as defined above, that is substituted with one or more hydroxy groups.

The term “animal” as used herein includes, but is not limited to, humans and non-human vertebrates such as wild, domestic and farm animals. As used herein, the terms “subject,” “subject” and “subject” are intended to include living organisms in which certain conditions as described herein can occur. Examples include humans, monkeys, cows, sheep, goats, dogs, cats, mice, rats, and transgenic species thereof. In a preferred embodiment, the subject is a primate. In certain embodiments, the primate or subject is a human.

In certain instances, the human is an adult. In certain instances, the human is child. In further instances, the human is under the age of 12 years. In certain instances, the human is elderly. In other instances, the human is 60 years of age or older. Other examples of subjects include experimental animals such as mice, rats, dogs, cats, goats, sheep, pigs, and cows. The experimental animal can be an animal model for a disorder, e.g., a transgenic mouse with hypertensive pathology.

A “cyano” group refers to a —CN group.

The term “EGFR,” as used herein, means the human protein known to those of ordinary skill in the art as epidermal growth factor receptor, and that is encoded by the EGFR gene. The terms “C797S,” “EGFR C797S,” “EGFR C797S mutation, “C797S mutation,” and the like mean the human EGFR protein having a mutation in which a cysteine amino acid at position 797 of the protein is replaced by the amino acid serine. The terms “L858R,” “EGFR L858R,” “EGFR L858R mutation, “L858R mutation,” and the like mean the human EGFR protein having a mutation in which a leucine amino acid at position 858 of the protein is replaced by arginine. The terms “L858R/C797S,” “L858R/C797S mutation,” and the like mean the human EGFR protein having two mutations in which a cysteine amino acid residue at position 797 of the protein is replaced by the amino acid serine, and a leucine amino acid residue at position 858 of the protein is replaced by arginine. The terms “exon 19 deletion,” “del19,” “del19 mutation,” and the like mean the human EGFR protein having one or more amino acid residues missing in the portion of the protein that is encoded by the portion of the EGFR gene found on exon 19 of the human genome. In some embodiments, the exon19 deletion mutations comprise one or more of delE746_A750; delE746_T751insA; delE746_T751insI; delE746_S752insI; delE746_S752insV; delL747_E749; delL747 A750insP; delL747_T751insP; delL747_S752; delL747_P753insQ; delL747_T751; delL747_P753insS 11; delL747_A755insSRD; delE746_P753insVS; delL747-P753; and delS752_I759. In some embodiments, the exon19 deletion is delE746_A750. In some embodiments, the exon19 deletion is delE746_T751insA. In some embodiments, the exon19 deletion is delE746_T751insI. In some embodiments, the exon19 deletion is delE746_S752insI.

In some embodiments, the exon19 deletion is delE746_S752insV. In some embodiments, the exon19 deletion is delL747_E749. In some embodiments, the exon19 deletion is delL747_A750insP. In some embodiments, the exon19 deletion is delL747_T751insP. In some embodiments, the exon19 deletion is delL747_S752. In some embodiments, the exon19 deletion is delL747_P753insQ. In some embodiments, the exon19 deletion is delL747_T751. In some embodiments, the exon19 deletion is delL747_P753insS 11. In some embodiments, the exon19 deletion is delL747_A755insSRD. In some embodiments, the exon19 deletion is delE746_P753insVS. In some embodiments, the exon19 deletion is delL747-P753. In some embodiments, the exon19 deletion is and delS752_I759. The terms “del19/C797S,” “del19/C797S mutations,” and the like mean the human EGFR protein having two or more mutations comprising an exon 19 deletion and one in which a cysteine amino acid residue at position 797 of the protein is replaced by the amino acid serine. The terms “T790M,” “EGFR T790M,” “EGFR T790M mutation,” “T790M mutation,” and the like mean the human EGFR protein having a mutation in which a threonine amino acid at position 790 of the protein is replaced by methionine.

In some embodiments, the exon19 deletion mutations comprise one or more of delE746_A750, delE746_T750insRP, delE746_T751insA, delE746_T751insI, delE746_T751insVA, delE746_S752insI, delE746_S752insA, delE746_S752insV, delL747_E749 (A750P), delL747 A750insP, delL747_T75insP, delL747_T751insS, delL747_S752, delL747_S752 (E746V), delL747_P753insQ, delL747_P752insQ, delL747_T751, delL747_P753insS, delL747 A755insSRD, delE746_P753insVS, delE746_P753insS, delL747-P753, and delS752_I759.

The term “halo” or “halogen,” as used herein, refers to bromo, chloro, fluoro or iodo. In some embodiments, halogen is fluoro or chloro. In some embodiments, halogen is fluoro.

The term “heterocycloalkyl,” as used herein, refers to a 3- to 24-membered partially or fully saturated ring radical comprising 2 to 23 carbon atoms and from one to 8 heteroatoms selected from boron, nitrogen, oxygen, phosphorous, and sulfur. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. In some embodiments, the heterocycloalkyl is a 3- to 6-membered heterocycloalkyl. In some embodiments, the heterocycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxothiomorpholinyl, 1,1-dioxothiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring).

The term “C₁-C₆heteroalkyl,” as used herein, means an alkyl group in which one or more skeletal atoms of the alkyl are selected from an atom other than carbon, e.g., boron, oxygen, nitrogen (e.g. —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl.

The term “heteroaryl,” as used herein refers to a 5- to 14-membered ring system radical comprising hydrogen atoms, one to thirteen carbon atoms, one to six heteroatoms selected from boron, nitrogen, oxygen, phosphorous, and sulfur, and at least one aromatic ring. The heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl).

By “pharmaceutically acceptable,” as used herein, is meant the carrier, diluent or excipient must be compatible with the other ingredients of the formulation and not deleterious to the recipient thereof.

The term “pharmaceutical composition” means a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “pharmaceutically acceptable salt,” as used herein, means a salt of a compound of the present invention that retains the biological effectiveness of the free acids and bases of the specified derivative and that is not biologically or otherwise undesirable.

The term “oxo,” as used herein, refers to a carbonyl moiety such that alkyl substituted by oxo refers to a ketone group.

The term “solvate,” as used herein, means a molecular complex between compounds of the present invention and solvent molecules. Examples of solvates include, but are not limited to, compounds of the invention in combination water, isopropanol, ethanol, methanol, dimethylsulfoxide (DMSO), ethyl acetate, acetic acid, ethanolamine, or mixtures thereof. The term “hydrate” can be used when said solvent is water. It is specifically contemplated that in the present invention one solvent molecule can be associated with one molecule of the compounds of the present invention, such as a hydrate. Furthermore, it is specifically contemplated that in the present invention, more than one solvent molecule may be associated with one molecule of the compounds of the present invention, such as a dihydrate. Additionally, it is specifically contemplated that in the present invention less than one solvent molecule may be associated with one molecule of the compounds of the present invention, such as a hemihydrate. Furthermore, solvates of the present invention are contemplated as solvates of compounds of the present invention that retain the biological effectiveness of the non-hydrate form of the compounds.

Where a compound of the invention contains an alkenyl group, geometric cis/trans (or Z/E) isomers are possible. Where the compound contains, for example, a keto or oxime group or an aromatic moiety, tautomeric isomerism (‘tautomerism’) can occur. Examples of tautomerism include keto and enol tautomers. A single compound may exhibit more than one type of isomerism. Included within the scope of the invention are all stereoisomers, geometric isomers, and tautomeric forms of the inventive compounds, including compounds exhibiting more than one type of isomerism, and mixtures of one or more thereof. Cis/trans isomers may be separated by conventional techniques well known to those skilled in the art, for example, chromatography and fractional crystallization.

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another. The terms “racemic” or “racemic mixture,” as used herein, refer to a 1:1 mixture of enantiomers of a particular compound. A mixture of racemates in which one racemate is present in a greater amount than the other racemate in such mixture may be described as “enantiomerically enriched.” The term “diastereomers”, on the other hand, refers to the relationship between a pair of stereoisomers that comprise two or more asymmetric centers and are not mirror images of one another. Designations that are conventional in the art may be used to describe stereoisomers of compounds, or the stereochemistry of a particular asymmetric carbon atom, of the compounds disclosed herein, or mixtures thereof. For example, a single racemate or stereocenter of a compound, may be described as of the (+), the (−), the (R)-, or the (S) configuration. A mixture of racemates may be described by use of the (±) symbol.

The compounds of the present invention may have asymmetric carbon atoms. The carbon-carbon bonds of the compounds of the present invention may be depicted herein using a solid line (−), a solid wedge ( [symbol] ) or a dotted wedge ( [symbol] ). The use of a solid line to depict bonds to asymmetric carbon atoms is meant to indicate that all possible stereoisomers (e.g. specific enantiomers, racemic mixtures, etc.) at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. For example, unless stated otherwise, it is intended that the compounds of the present invention can exist as enantiomers and diastereomers or as racemates and mixtures thereof. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

Conventional techniques for the preparation/isolation of individual enantiomers include chiral synthesis from a suitable optically pure precursor or resolution of the racemate using, for example, chiral HPLC. Alternatively, the racemate (or a racemic precursor) may be reacted with a suitable optically active compound, for example, an alcohol, or, in the case where the compound contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to one skilled in the art. Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% isopropanol, typically from 2 to 20%, and from 0 to 5% of an alkylamine, typically 0.1% diethylamine. Concentration of the eluate affords the enriched mixture. Stereoisomeric conglomerates may be separated by conventional techniques known to those skilled in the art. See, e.g. “Stereochemistry of Organic Compounds” by E L Eliel (Wiley, New York, 1994), the disclosure of which is incorporated herein by reference in its entirety.

The term “absolute stereochemistry arbitrarily assigned” as used herein means the absolute stereochemistry of one or more stereocenters in a compound has been drawn in a particular way (e.g., the “R” or “S” configuration) for convenience even though the absolute stereochemistry of one or more stereocenters present in the compound has not been experimentally determined. In such cases, one of ordinary skill in the art would understand that a compound described as “absolute stereochemistry arbitrarily assigned” and depicted as the “R” enantiomer may be the “S” enantiomer; and a compound described as “absolute stereochemistry arbitrarily assigned” and depicted as the “S” enantiomer may be the “R” enantiomer. As such, the use of the (S)- and (R)-designations in those compounds described as “absolute stereochemistry arbitrarily assigned” is not intended to define the absolute optical rotation of the designated bond or compound. If desired, the absolute stereochemistry of stereocenters in a compound can be determined using methods known to those having ordinary skill in the art, such as single molecule x-ray diffraction.

The term “substituted,” as used herein, means that the specified group or moiety bears one or more substituents. The term “unsubstituted,” means that the specified group bears no substituents. The term “optionally substituted” means that the specified group is unsubstituted or substituted by one or more substituents. It is to be understood that in the compounds of the present invention when a group is said to be “unsubstituted,” or is “substituted” with fewer groups than would fill the valencies of all the atoms in the compound, the remaining valencies on such a group are filled by hydrogen. For example, if a C₆aryl group, also called “phenyl” herein, is substituted with one additional substituent, one of ordinary skill in the art would understand that such a group has 4 open positions left on carbon atoms of the C₆aryl group (6 initial positions, minus one to which the remainder of the compound of the present invention is bonded, minus an additional substituent, to leave 4). In such cases, the remaining 4 carbon atoms are each bound to one hydrogen atom to fill their valencies. Similarly, if a C₆aryl group in the present compounds is said to be “disubstituted,” one of ordinary skill in the art would understand it to mean that the C₆aryl group has 3 carbon atoms remaining that are unsubstituted. Those three unsubstituted carbon atoms are each bound to one hydrogen atom to fill their valencies.

In accordance with a convention used in the art, the symbol

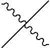

is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g.,

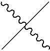

represents a methyl group,

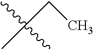 [SMILES: *CC]

represents an ethyl group, and

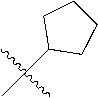 [SMILES: *C1CCCC1]

represents a cyclopentyl group, etc.

If a group, as for example, (R¹)ₐ is depicted as “floating” on a group that is a ring, such as when A is defined as a ring in the formula:

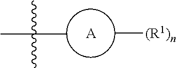

then, unless otherwise defined, the substituent R¹ may reside on any atom of the ring system, assuming replacement of a depicted, implied, or expressly defined hydrogen from one of the ring atoms, so long as a stable structure is formed. A ring system A may be, for example, but not limited to aryl, heteroaryl, cycloalkyl, heterocycloalkyl, spirocyclyl, or a fused ring system.

It is understood that if the compounds of Formula (I), (II), (IIa), (IIb), (IIc), (IId), (IIe), (III), (IV), (V), (VI), (VII), (VIII), (VIIIa), (IX), (X), (XI), or (XII) are depicted as having an “R” group “floating” on a ring system (e.g., “m” and/or “n” in the compounds of Formula (I)), if m and/or n is less than the number of substitutable atoms the ring system depicted, the other substitutable positions on that ring system are bonded to a hydrogen atom. For example, in the compounds of Formula (I), if m is defined as equal to zero, then it is understood that the five substitutable carbon atoms on the phenyl ring are each bound to hydrogen. In a further example, if in the compounds of Formula (I) m is defined as equal to one, then it is understood that the remaining four substitutable carbon atoms on the phenyl ring are each bound to hydrogen. In yet another example, if in the compounds of Formula (I), ring B is defined as phenyl, and n is defined as equal to zero, then it is understood that the five substitutable carbon atoms on the phenyl ring are each bound to hydrogen. In still another example, if in the compounds of Formula (I), ring B is defined as pyridinyl, and n is defined as equal to one, then it is understood that the three remaining substitutable carbon atoms on the pyridinyl ring are each bound to hydrogen.

As used herein, the term “therapeutic” means an agent utilized to treat, combat, ameliorate, prevent, or improve an unwanted condition or disease of a subject.

A “therapeutically effective amount” or “effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in a subject that may be predisposed to the disease, condition or disorder but does not yet experience or display the pathology or symptomatology of the disease, (2) inhibiting the disease; for example, inhibiting a disease, condition or disorder in a subject that is experiencing or displaying the pathology or symptomatology of the disease, condition or disorder (i.e., arresting further development of the pathology and/or symptomatology), and (3) ameliorating the disease; for example, ameliorating a disease, condition or disorder in a subject that is experiencing or displaying the pathology or symptomatology of the disease, condition or disorder (i.e., reversing the pathology and/or symptomatology).

The terms “treat,” “treated,” “treatment,” or “treating” as used herein refers to both therapeutic treatment in some embodiments and prophylactic or preventative measures in other embodiments, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. A prophylactic benefit of treatment includes prevention of a condition, retarding the progress of a condition, stabilization of a condition, or decreasing the likelihood of occurrence of a condition. As used herein, “treat,” “treated,” “treatment,” or “treating” includes prophylaxis in some embodiments.

Further provided herein are compounds selected from those set forth in Table 1.

TABLE 1

| Example | | |
|---|---|---|
| No. | Structure | Name |
| 1 | 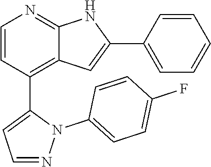 [SMILES: Fc1ccc(-n2nccc2-c2ccnc3[nH]c(-c4ccccc4)cc23)cc1] | 4-[1-(4-fluorophenyl)-1H- pyrazol-5-yl]-2-phenyl-1H- pyrrolo[2,3-b]pyridine |
| 2 | 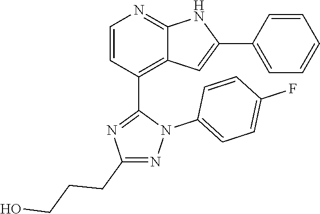 [SMILES: OCCCc1nc(-c2ccnc3[nH]c(-c4ccccc4)cc23)n(-c2ccc(F)cc2)n1] | 3-[1-(4-fluorophenyl)-5-(2- phenyl-1H-pyrrolo[2,3- b]pyridin-4-yl)-1H-1,2,4-triazol- 3-yl]propan-1-ol |
| 3 | 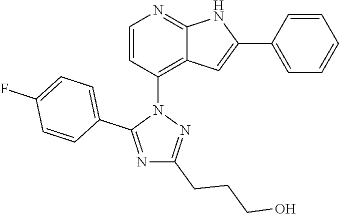 [SMILES: OCCCc1nc(-c2ccc(F)cc2)n(-c2ccnc3[nH]c(-c4ccccc4)cc23)n1] | 3-[5-(4-fluorophenyl)-1-(2- phenyl-1H-pyrrolo[2,3- b]pyridin-4-yl)-1H-1,2,4-triazol- 3-yl]propan-1-ol |
| 4 | 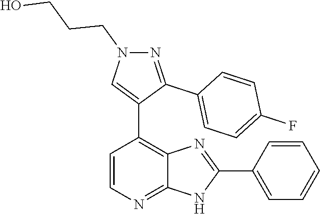 [SMILES: OCCCn1cc(-c2ccnc3[nH]c(-c4ccccc4)nc23)c(-c2ccc(F)cc2)n1] | 3-[3-(4-fluorophenyl)-4-(2- phenyl-3H-imidazo[4,5- b]pyridin-7-yl)-1H-pyrazol-1- yl]propan-1-ol |
| 5 | 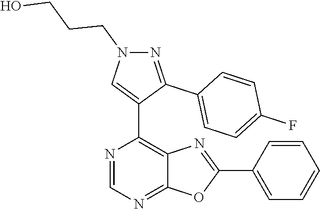 [SMILES: OCCCn1cc(-c2ncnc3oc(-c4ccccc4)nc23)c(-c2ccc(F)cc2)n1] | 3-[3-(4-fluorophenyl)-4-(2- phenyl[1,3]oxazolo[5,4- d]pyrimidin-7-yl)-1H-pyrazol-1- yl]propan-1-ol |
| 6 | 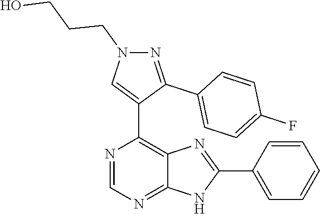 [SMILES: OCCCn1cc(-c2ncnc3[nH]c(-c4ccccc4)nc23)c(-c2ccc(F)cc2)n1] | 3-[3-(4-fluorophenyl)-4-(8- phenyl-9H-purin-6-yl)-1H- pyrazol-1-yl]propan-1-ol |
| 7 | 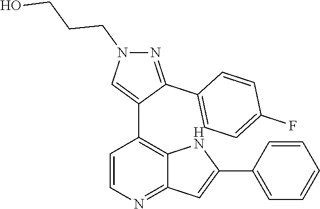 [SMILES: OCCCn1cc(-c2ccnc3cc(-c4ccccc4)[nH]c23)c(-c2ccc(F)cc2)n1] | 3-[3-(4-fluorophenyl)-4-(2- phenyl-1H-pyrrolo[3,2- b]pyridin-7-yl)-1H-pyrazol-1- yl]propan-1-ol |
| 8 | 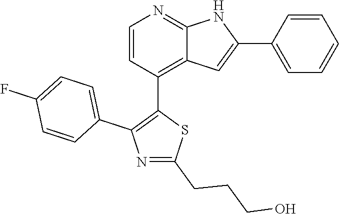 [SMILES: OCCCc1nc(-c2ccc(F)cc2)c(-c2ccnc3[nH]c(-c4ccccc4)cc23)s1] | 3-[4-(4-fluorophenyl)-5-(2- phenyl-1H-pyrrolo[2,3- b]pyridin-4-yl)-1,3-thiazol-2- yl]propan-1-ol |
| 9 | 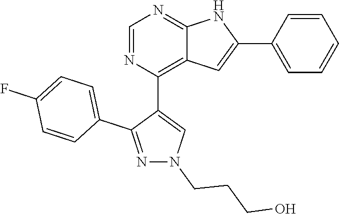 [SMILES: OCCCn1cc(-c2ncnc3[nH]c(-c4ccccc4)cc23)c(-c2ccc(F)cc2)n1] | 3-[3-(4-fluorophenyl)-4-(6- phenyl-7H-pyrrolo[2,3- d]pyrimidin-4-yl)-1H-pyrazol-1- yl]propan-1-ol |
| 10 | 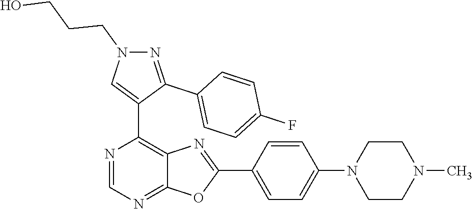 [SMILES: CN1CCN(c2ccc(-c3nc4c(-c5cn(CCCO)nc5-c5ccc(F)cc5)ncnc4o3)cc2)CC1] | 3-[3-(4-fluorophenyl)-4-{2-[4- (4-methylpiperazin-1- yl)phenyl][1,3]oxazolo[5,4- d]pyrimidin-7-yl}-1H-pyrazol-1- yl]propan-1-ol |
| 11 | 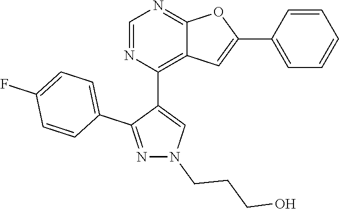 [SMILES: OCCCn1cc(-c2ncnc3oc(-c4ccccc4)cc23)c(-c2ccc(F)cc2)n1] | 3-[3-(4-fluorophenyl)-4-(6- phenylfuro[2,3-d]pyrimidin-4- yl)-1H-pyrazol-1-yl]propan-1-ol |
| 12 | 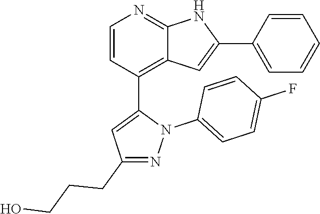 [SMILES: OCCCc1cc(-c2ccnc3[nH]c(-c4ccccc4)cc23)n(-c2ccc(F)cc2)n1] | 3-[1-(4-fluorophenyl)-5-(2- phenyl-1H-pyrrolo[2,3- b]pyridin-4-yl)-1H-pyrazol-3- yl]propan-1-ol |
| 13 | 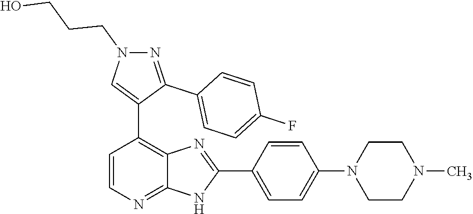 [SMILES: CN1CCN(c2ccc(-c3nc4c(-c5cn(CCCO)nc5-c5ccc(F)cc5)ccnc4[nH]3)cc2)CC1] | 3-[3-(4-fluorophenyl)-4-{2-[4- (4-methylpiperazin-1-yl)phenyl]- 3H-imidazo[4,5-b]pyridin-7-yl}- 1H-pyrazol-1-yl]propan-1-ol |
| 14 | 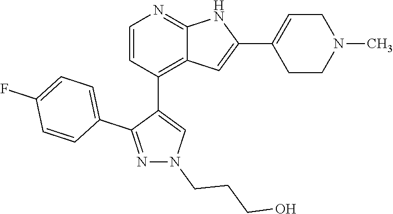 [SMILES: CN1CC=C(c2cc3c(-c4cn(CCCO)nc4-c4ccc(F)cc4)ccnc3[nH]2)CC1] | 3-{3-(4-fluorophenyl)-4-[2-(1- methyl-1,2,3,6- tetrahydropyridin-4-yl)-1H- pyrrolo[2,3-b]pyridin-4-yl]-1H- pyrazol-1-yl}propan-1-ol |
| 15 | 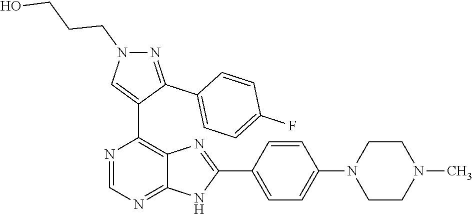 [SMILES: CN1CCN(c2ccc(-c3nc4c(-c5cn(CCCO)nc5-c5ccc(F)cc5)ncnc4[nH]3)cc2)CC1] | 3-[3-(4-fluorophenyl)-4-{8-[4- (4-methylpiperazin-1-yl)phenyl]- 9H-purin-6-y1}-1H-pyrazol-1- yl]propan-1-ol |
| 16 | 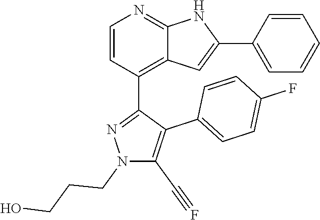 | 4-(4-fluorophenyl)-1-(3- hydroxypropyl)-3-(2-phenyl-1H- pyrrolo[2,3-b]pyridin-4-yl)-1H- pyrazole-5-carbonitrile |
| 17 | 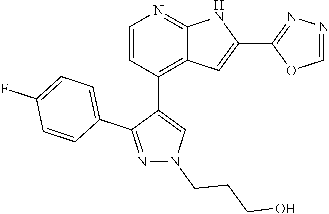 [SMILES: OCCCn1cc(-c2ccnc3[nH]c(-c4nnco4)cc23)c(-c2ccc(F)cc2)n1] | 3-{3-(4-fluorophenyl)-4-[2- (1,3,4-oxadiazol-2-yl)-1H- pyrrolo[2,3-b]pyridin-4-yl]-1H- pyrazol-1-yl}propan-1-ol |
| 18 | 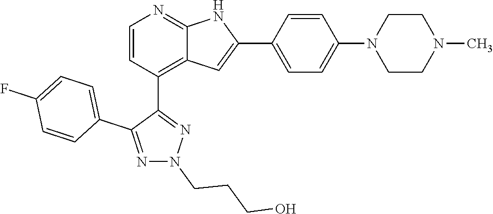 [SMILES: CN1CCN(c2ccc(-c3cc4c(-c5nn(CCCO)nc5-c5ccc(F)cc5)ccnc4[nH]3)cc2)CC1] | 3-[4-(4-fluorophenyl)-5-{2-[4- (4-methylpiperazin-1-yl)phenyl]- 1H-pyrrolo[2,3-b]pyridin-4-yl}- 2H-1,2,3-triazol-2-yl]propan-1- ol |
| 19 | 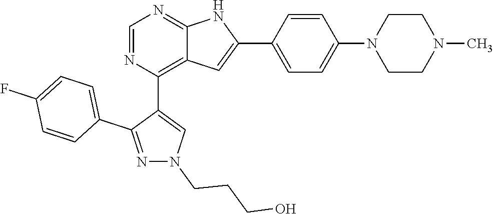 [SMILES: CN1CCN(c2ccc(-c3cc4c(-c5cn(CCCO)nc5-c5ccc(F)cc5)ncnc4[nH]3)cc2)CC1] | 3-[3-(4-fluorophenyl)-4-{6-[4- (4-methylpiperazin-1-yl)phenyl]- 7H-pyrrolo[2,3-d]pyrimidin-4- yl}-1H-pyrazol-1-yl]propan-1-ol |
| 20 | 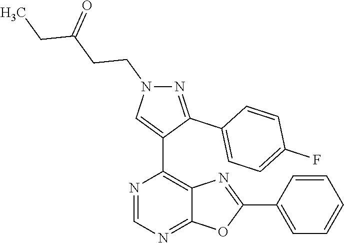 [SMILES: CCC(=O)CCn1cc(-c2ncnc3oc(-c4ccccc4)nc23)c(-c2ccc(F)cc2)n1] | 1-[3-(4-fluorophenyl)-4-(2- phenyl[1,3]oxazolo[5,4- d]pyrimidin-7-yl)-1H-pyrazol-1- yl]pentan-3-one |
| 21 | 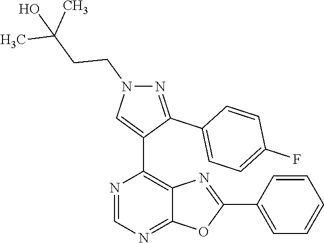 [SMILES: CC(C)(O)CCn1cc(-c2ncnc3oc(-c4ccccc4)nc23)c(-c2ccc(F)cc2)n1] | 4-[3-(4-fluorophenyl)-4-(2- phenyl[1,3]oxazolo[5,4- d]pyrimidin-7-yl)-1H-pyrazol-1- yl]-2-methylbutan-2-ol |
| 22 | 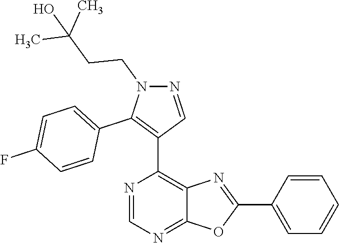 [SMILES: CC(C)(O)CCn1ncc(-c2ncnc3oc(-c4ccccc4)nc23)c1-c1ccc(F)cc1] | 4-[5-(4-fluorophenyl)-4-(2- phenyl[1,3]oxazolo[5,4- d]pyrimidin-7-yl)-1H-pyrazol-1- yl]-2-methylbutan-2-ol |
| 23 | 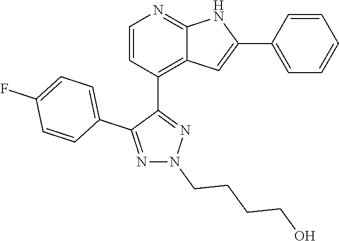 [SMILES: OCCCCn1nc(-c2ccc(F)cc2)c(-c2ccnc3[nH]c(-c4ccccc4)cc23)n1] | 4-[4-(4-fluorophenyl)-5-(2- phenyl-1H-pyrrolo[2,3- b]pyridin-4-yl)-2H-1,2,3-triazol- 2-yl]butan-1-ol |
| 24 | 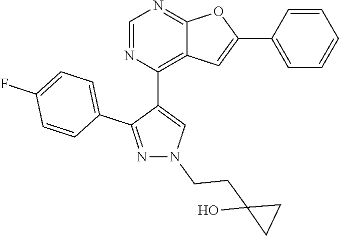 [SMILES: OC1(CCn2cc(-c3ncnc4oc(-c5ccccc5)cc34)c(-c3ccc(F)cc3)n2)CC1] | 1-{2-[3-(4-fluorophenyl)-4-(6- phenylfuro[2,3-d]pyrimidin-4- yl)-1H-pyrazol-1- yl]ethyl}cyclopropan-1-ol |
| 25 | 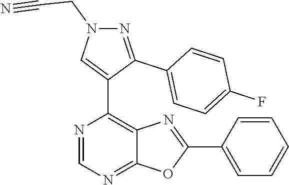 [SMILES: N#CCn1cc(-c2ncnc3oc(-c4ccccc4)nc23)c(-c2ccc(F)cc2)n1] | [3-(4-fluorophenyl)-4-(2- phenyl[1,3]oxazolo[5,4- d]pyrimidin-7-yl)-1H-pyrazol-1- yl]acetonitrile |
| 26 | 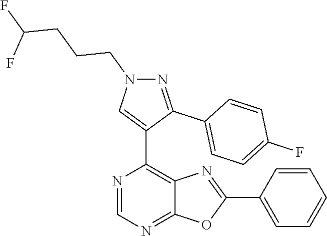 [SMILES: Fc1ccc(-c2nn(CCCC(F)F)cc2-c2ncnc3oc(-c4ccccc4)nc23)cc1] | 7-[1-(4,4-difluorobutyl)-3-(4- fluorophenyl)-1H-pyrazol-4-yl]- 2-phenyl[1,3]oxazolo[5,4- d]pyrimidine |
| 27 | 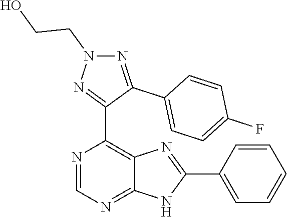 [SMILES: OCCn1nc(-c2ccc(F)cc2)c(-c2ncnc3[nH]c(-c4ccccc4)nc23)n1] | 2-[4-(4-fluorophenyl)-5-(8- phenyl-9H-purin-6-yl)-2H-1,2,3- triazol-2-yl]ethan-1-ol |
| 28 | 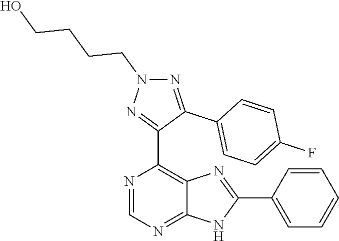 [SMILES: OCCCCn1nc(-c2ccc(F)cc2)c(-c2ncnc3[nH]c(-c4ccccc4)nc23)n1] | 4-[4-(4-fluorophenyl)-5-(8- phenyl-9H-purin-6-yl)-2H-1,2,3- triazol-2-yl]butan-1-ol |
| 29 | 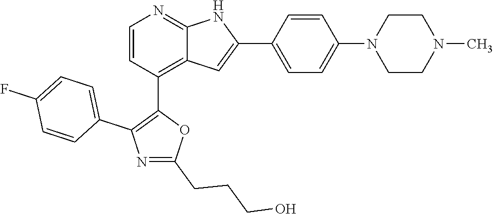 [SMILES: CN1CCN(c2ccc(-c3cc4c(-c5oc(CCCO)nc5-c5ccc(F)cc5)ccnc4[nH]3)cc2)CC1] | 3-[4-(4-fluorophenyl)-5-{2-[4- (4-methylpiperazin-1-yl)phenyl]- 1H-pyrrolo[2,3-b]pyridin-4-yl}- 1,3-oxazol-2-yl]propan-1-ol |
| 30 | 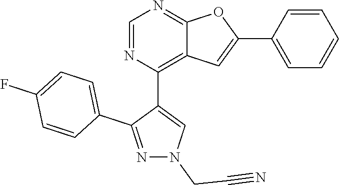 [SMILES: N#CCn1cc(-c2ncnc3oc(-c4ccccc4)cc23)c(-c2ccc(F)cc2)n1] | [3-(4-fluorophenyl)-4-(6- phenylfuro[2,3-d]pyrimidin-4- yl)-1H-pyrazol-1-yl]acetonitrile |
| 31 | 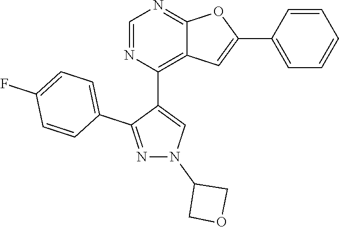 [SMILES: Fc1ccc(-c2nn(C3COC3)cc2-c2ncnc3oc(-c4ccccc4)cc23)cc1] | 4-[3-(4-fluorophenyl)-1-(oxetan- 3-yl)-1H-pyrazol-4-yl]-6- phenylfuro[2,3-d]pyrimidine |
| 32 | 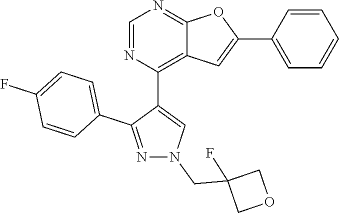 [SMILES: Fc1ccc(-c2nn(CC3(F)COC3)cc2-c2ncnc3oc(-c4ccccc4)cc23)cc1] | 4-{1-[(3-fluorooxetan-3- yl)methyl]-3-(4-fluorophenyl)- 1H-pyrazol-4-yl}-6- phenylfuro[2,3-d]pyrimidine |
| 33 | 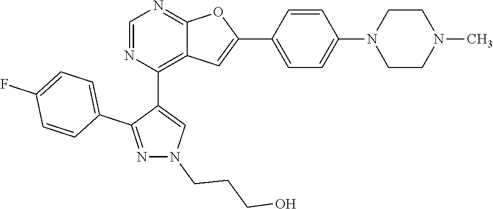 [SMILES: CN1CCN(c2ccc(-c3cc4c(-c5cn(CCCO)nc5-c5ccc(F)cc5)ncnc4o3)cc2)CC1] | 3-[3-(4-fluorophenyl)-4-{6-[4- (4-methylpiperazin-1- yl)phenyl]furo[2,3-d]pyrimidin- 4-yl}-1H-pyrazol-1-yl]propan-1- ol |
| 34 | 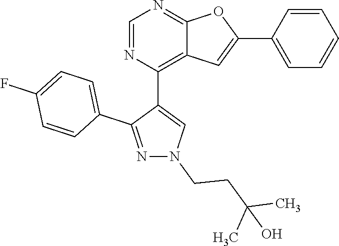 [SMILES: CC(C)(O)CCn1cc(-c2ncnc3oc(-c4ccccc4)cc23)c(-c2ccc(F)cc2)n1] | 4-[3-(4-fluorophenyl)-4-(6- phenylfuro[2,3-d]pyrimidin-4- yl)-1H-pyrazol-1-yl]-2- methylbutan-2-ol |
| 35 | 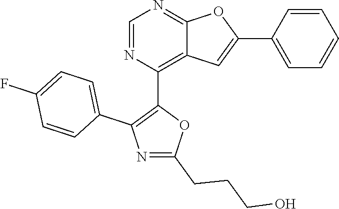 [SMILES: OCCCc1nc(-c2ccc(F)cc2)c(-c2ncnc3oc(-c4ccccc4)cc23)o1] | 3-[4-(4-fluorophenyl)-5-(6- phenylfuro[2,3-d]pyrimidin-4- yl)-1,3-oxazol-2-yl]propan-1-ol |
| 36 | 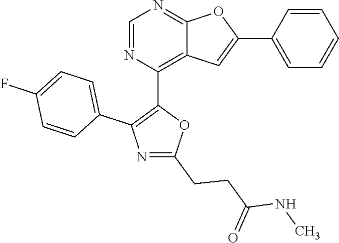 [SMILES: CNC(=O)CCc1nc(-c2ccc(F)cc2)c(-c2ncnc3oc(-c4ccccc4)cc23)o1] | 3-[4-(4-fluorophenyl)-5-(6- phenylfuro[2,3-d]pyrimidin-4- yl)-1,3-oxazol-2-yl]-N- methylpropanamide |
| 37 | 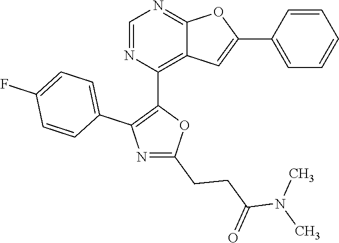 [SMILES: CN(C)C(=O)CCc1nc(-c2ccc(F)cc2)c(-c2ncnc3oc(-c4ccccc4)cc23)o1] | 3-[4-(4-fluorophenyl)-5-(6- phenylfuro[2,3-d]pyrimidin-4- yl)-1,3-oxazol-2-yl]-N,N- dimethylpropanamide |
| 38 | 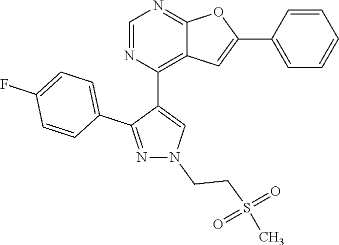 [SMILES: CS(=O)(=O)CCn1cc(-c2ncnc3oc(-c4ccccc4)cc23)c(-c2ccc(F)cc2)n1] | 4-{3-(4-fluorophenyl)-1-[2- (methanesulfonyl)ethyl]-1H- pyrazol-4-yl}-6-phenylfuro[2,3- d]pyrimidine |
| 39 | 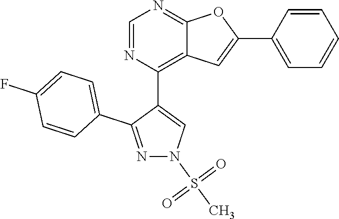 [SMILES: CS(=O)(=O)n1cc(-c2ncnc3oc(-c4ccccc4)cc23)c(-c2ccc(F)cc2)n1] | 4-[3-(4-fluorophenyl)-1- (methanesulfonyl)-1H-pyrazol-4- yl]-6-phenylfuro[2,3- d]pyrimidine |
| 40 | 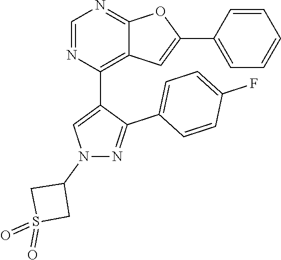 [SMILES: O=S1(=O)CC(n2cc(-c3ncnc4oc(-c5ccccc5)cc34)c(-c3ccc(F)cc3)n2)C1] | 3-[3-(4-fluorophenyl)-4-(6- phenylfuro[2,3-d]pyrimidin-4- yl)-1H-pyrazol-1-yl]-1λ6- thietane-1,1-dione |
| 41 | 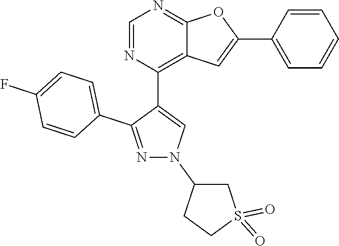 [SMILES: O=S1(=O)CCC(n2cc(-c3ncnc4oc(-c5ccccc5)cc34)c(-c3ccc(F)cc3)n2)C1] | 3-[3-(4-fluorophenyl)-4-(6- phenylfuro[2,3-d]pyrimidin-4- yl)-1H-pyrazol-1-yl]-1λ6- thiolane-1,1-dione |
| 42 | 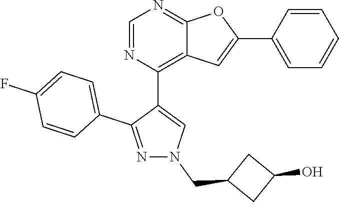 [SMILES: O[C@H]1C[C@@H](Cn2cc(-c3ncnc4oc(-c5ccccc5)cc34)c(-c3ccc(F)cc3)n2)C1] | (1s,3s)-3-{[3-(4-fluorophenyl)- 4-(6-phenylfuro[2,3-d]pyrimidin- 4-yl)-1H-pyrazol-1- yl]methyl}cyclobutan-1-ol |
| 43 | 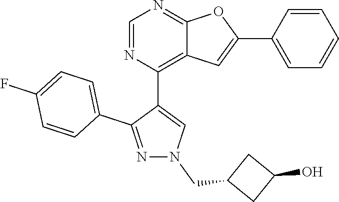 [SMILES: O[C@H]1C[C@H](Cn2cc(-c3ncnc4oc(-c5ccccc5)cc34)c(-c3ccc(F)cc3)n2)C1] | (1r,3r)-3-{[3-(4-fluorophenyl)-4- (6-phenylfuro[2,3-d]pyrimidin-4- yl)-1H-pyrazol-1- yl]methyl}cyclobutan-1-ol |
| 44 | 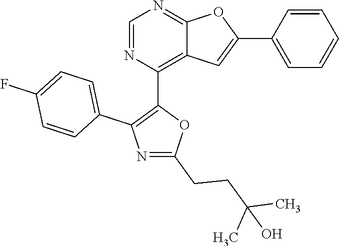 [SMILES: CC(C)(O)CCc1nc(-c2ccc(F)cc2)c(-c2ncnc3oc(-c4ccccc4)cc23)o1] | 4-[4-(4-fluorophenyl)-5-(6- phenylfuro[2,3-d]pyrimidin-4- yl)-1,3-oxazol-2-yl]-2- methylbutan-2-ol |
| 45 | 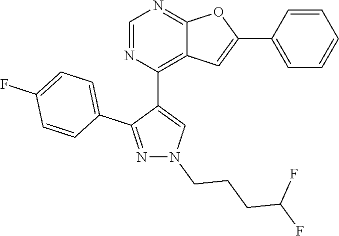 [SMILES: Fc1ccc(-c2nn(CCCC(F)F)cc2-c2ncnc3oc(-c4ccccc4)cc23)cc1] | 4-[1-(4,4-difluorobutyl)-3-(4- fluorophenyl)-1H-pyrazol-4-yl]- 6-phenylfuro[2,3-d]pyrimidine |
| 46 | 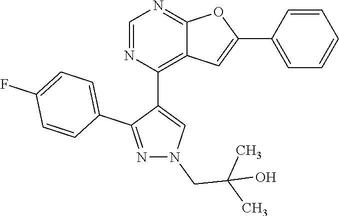 [SMILES: CC(C)(O)Cn1cc(-c2ncnc3oc(-c4ccccc4)cc23)c(-c2ccc(F)cc2)n1] | 1-[3-(4-fluorophenyl)-4-(6- phenylfuro[2,3-d]pyrimidin-4- yl)-1H-pyrazol-1-yl]-2- methylpropan-2-ol |
| 47 | 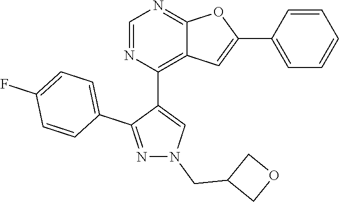 [SMILES: Fc1ccc(-c2nn(CC3COC3)cc2-c2ncnc3oc(-c4ccccc4)cc23)cc1] | 4-{3-(4-fluorophenyl)-1- [(oxetan-3-yl)methyl]-1H- pyrazol-4-yl}-6-phenylfuro[2,3- d]pyrimidine |
| 48 | 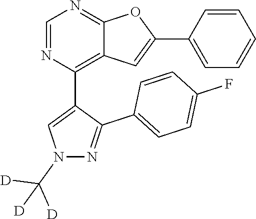 [SMILES: [2H]C([2H])([2H])n1cc(-c2ncnc3oc(-c4ccccc4)cc23)c(-c2ccc(F)cc2)n1] | 4-[3-(4-fluorophenyl)-1- (2H³)methyl-1H-pyrazol-4-yl]-6- phenylfuro[2,3-d]pyrimidine |
| 49 | 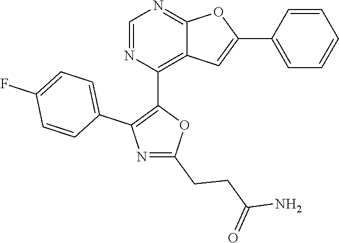 [SMILES: NC(=O)CCc1nc(-c2ccc(F)cc2)c(-c2ncnc3oc(-c4ccccc4)cc23)o1] | 3-[4-(4-fluorophenyl)-5-(6- phenylfuro[2,3-d]pyrimidin-4- yl)-1,3-oxazol-2-yl]propanamide |
| 50 | 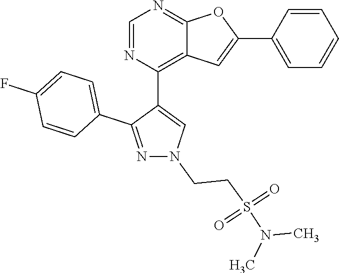 [SMILES: CN(C)S(=O)(=O)CCn1cc(-c2ncnc3oc(-c4ccccc4)cc23)c(-c2ccc(F)cc2)n1] | 2-[3-(4-fluorophenyl)-4-(6- phenylfuro[2,3-d]pyrimidin-4- yl)-1H-pyrazol-1-yl]-N,N- dimethylethane-1-sulfonamide |
| 51 | 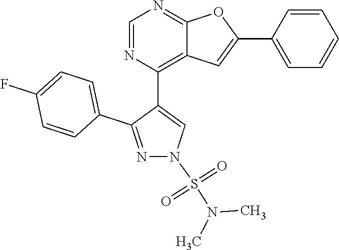 [SMILES: CN(C)S(=O)(=O)n1cc(-c2ncnc3oc(-c4ccccc4)cc23)c(-c2ccc(F)cc2)n1] | 3-(4-fluorophenyl)-N,N- dimethyl-4-(6-phenylfuro[2,3- d]pyrimidin-4-yl)-1H-pyrazole- 1-sulfonamide |
| 52 | 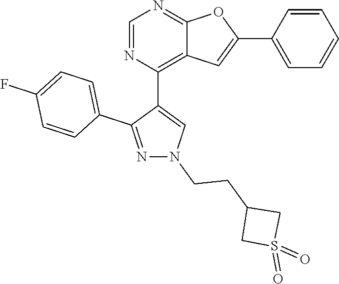 [SMILES: O=S1(=O)CC(CCn2cc(-c3ncnc4oc(-c5ccccc5)cc34)c(-c3ccc(F)cc3)n2)C1] | 3-{2-[3-(4-fluorophenyl)-4-(6- phenylfuro[2,3-d]pyrimidin-4- yl)-1H-pyrazol-1-yl]ethyl}-126- thietane-1,1-dione |
| 53 | 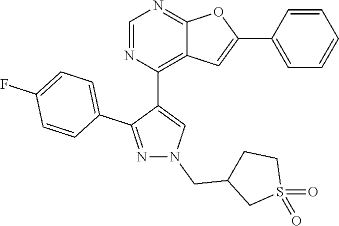 [SMILES: O=S1(=O)CCC(Cn2cc(-c3ncnc4oc(-c5ccccc5)cc34)c(-c3ccc(F)cc3)n2)C1] | 3-{[3-(4-fluorophenyl)-4-(6- phenylfuro[2,3-d]pyrimidin-4- yl)-1H-pyrazol-1-yl]methyl}- 1λ6-thiolane-1,1-dione |
| 54 | 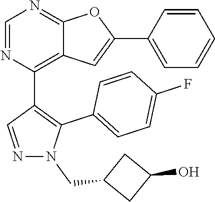 [SMILES: O[C@H]1C[C@H](Cn2ncc(-c3ncnc4oc(-c5ccccc5)cc34)c2-c2ccc(F)cc2)C1] | (1r,3r)-3-{[5-(4-fluorophenyl)-4- (6-phenylfuro[2,3-d]pyrimidin-4- yl)-1H-pyrazol-1- yl]methyl}cyclobutan-1-ol |
| 55 | 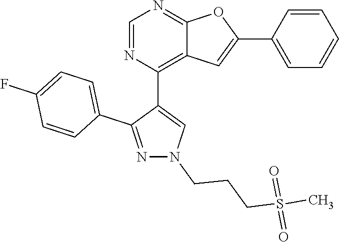 [SMILES: CS(=O)(=O)CCCn1cc(-c2ncnc3oc(-c4ccccc4)cc23)c(-c2ccc(F)cc2)n1] | 4-{3-(4-fluorophenyl)-1-[3- (methanesulfonyl)propyl]-1H- pyrazol-4-yl}-6-phenylfuro[2,3- d]pyrimidine |
| 56 | 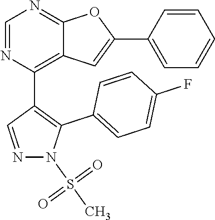 [SMILES: CS(=O)(=O)n1ncc(-c2ncnc3oc(-c4ccccc4)cc23)c1-c1ccc(F)cc1] | 4-[5-(4-fluorophenyl)-1- (methanesulfonyl)-1H-pyrazol-4- yl]-6-phenylfuro[2,3- d]pyrimidine |
| 57 | 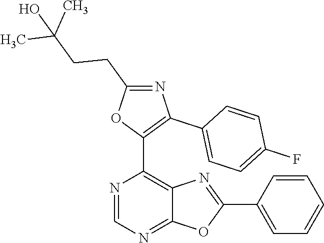 [SMILES: CC(C)(O)CCc1nc(-c2ccc(F)cc2)c(-c2ncnc3oc(-c4ccccc4)nc23)o1] | 4-[4-(4-fluorophenyl)-5-(2- phenyl[1,3]oxazolo[5,4- d]pyrimidin-7-yl)-1,3-oxazol-2- yl]-2-methylbutan-2-ol |
| 58 | 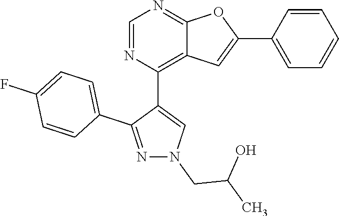 [SMILES: CC(O)Cn1cc(-c2ncnc3oc(-c4ccccc4)cc23)c(-c2ccc(F)cc2)n1] | 1-[3-(4-fluorophenyl)-4-(6- phenylfuro[2,3-d]pyrimidin-4- yl)-1H-pyrazol-1-yl]propan-2-ol |
| 59 | 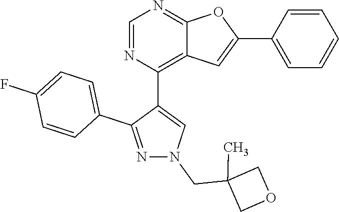 [SMILES: CC1(Cn2cc(-c3ncnc4oc(-c5ccccc5)cc34)c(-c3ccc(F)cc3)n2)COC1] | 4-{3-(4-fluorophenyl)-1-[(3- methyloxetan-3-yl)methyl]-1H- pyrazol-4-yl}-6-phenylfuro[2,3- d]pyrimidine |
| 60 | 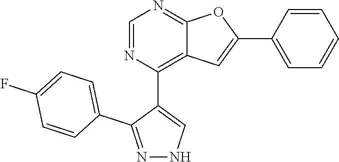 [SMILES: Fc1ccc(-c2n[nH]cc2-c2ncnc3oc(-c4ccccc4)cc23)cc1] | 4-[3-(4-fluorophenyl)-1H- pyrazol-4-yl]-6-phenylfuro[2,3- d]pyrimidine |
| 61 | 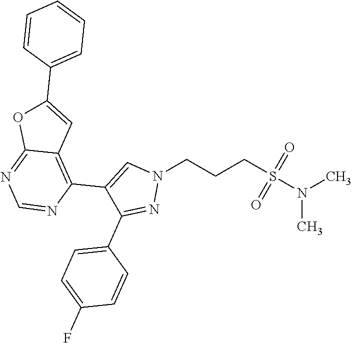 [SMILES: CN(C)S(=O)(=O)CCCn1cc(-c2ncnc3oc(-c4ccccc4)cc23)c(-c2ccc(F)cc2)n1] | 3-[3-(4-fluorophenyl)-4-(6- phenylfuro[2,3-d]pyrimidin-4- yl)-1H-pyrazol-1-yl]-N,N- dimethylpropane-1-sulfonamide |
| 62 | 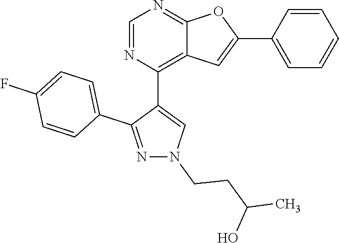 [SMILES: CC(O)CCn1cc(-c2ncnc3oc(-c4ccccc4)cc23)c(-c2ccc(F)cc2)n1] | 4-[3-(4-fluorophenyl)-4-(6- phenylfuro[2,3-d]pyrimidin-4- yl)-1H-pyrazol-1-yl]butan-2-ol |
| 63 | 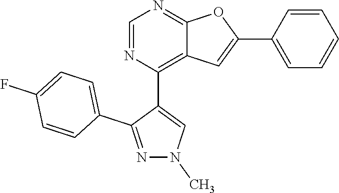 [SMILES: Cn1cc(-c2ncnc3oc(-c4ccccc4)cc23)c(-c2ccc(F)cc2)n1] | 4-[3-(4-fluorophenyl)-1-methyl- 1H-pyrazol-4-yl]-6- phenylfuro[2,3-d]pyrimidine |
| 64 | 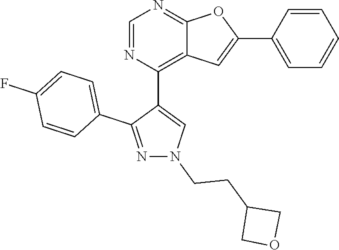 [SMILES: Fc1ccc(-c2nn(CCC3COC3)cc2-c2ncnc3oc(-c4ccccc4)cc23)cc1] | 4-{3-(4-fluorophenyl)-1-[2- (oxetan-3-yl)ethyl]-1H-pyrazol- 4-yl}-6-phenylfuro[2,3- d]pyrimidine |
| 65 | 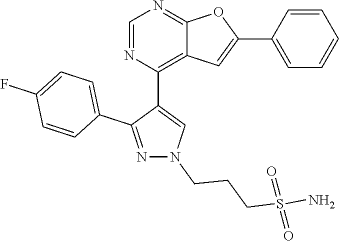 [SMILES: NS(=O)(=O)CCCn1cc(-c2ncnc3oc(-c4ccccc4)cc23)c(-c2ccc(F)cc2)n1] | 3-[3-(4-fluorophenyl)-4-(6- phenylfuro[2,3-d]pyrimidin-4- yl)-1H-pyrazol-1-yl]propane-1- sulfonamide |
| 66 | 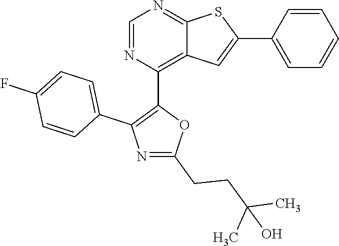 [SMILES: CC(C)(O)CCc1nc(-c2ccc(F)cc2)c(-c2ncnc3sc(-c4ccccc4)cc23)o1] | 4-[4-(4-fluorophenyl)-5-(6- phenylthieno[2,3-d]pyrimidin-4- yl)-1,3-oxazol-2-yl]-2- methylbutan-2-ol |
| 67 | 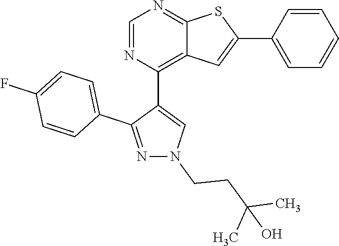 [SMILES: CC(C)(O)CCn1cc(-c2ncnc3sc(-c4ccccc4)cc23)c(-c2ccc(F)cc2)n1] | 4-[3-(4-fluorophenyl)-4-(6- phenylthieno[2,3-d]pyrimidin-4- yl)-1H-pyrazol-1-yl]-2- methylbutan-2-ol |
| 68 | 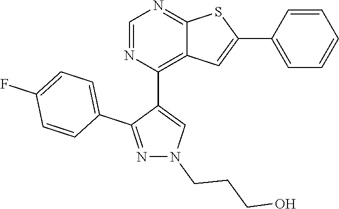 [SMILES: OCCCn1cc(-c2ncnc3sc(-c4ccccc4)cc23)c(-c2ccc(F)cc2)n1] | 3-[3-(4-fluorophenyl)-4-(6- phenylthieno[2,3-d]pyrimidin-4- yl)-1H-pyrazol-1-yl]propan-1-ol |
| 69 | 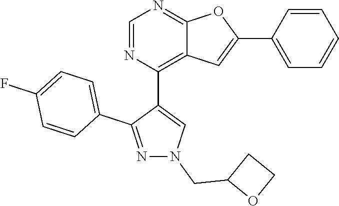 [SMILES: Fc1ccc(-c2nn(CC3CCO3)cc2-c2ncnc3oc(-c4ccccc4)cc23)cc1] | 4-{3-(4-fluorophenyl)-1- [(oxetan-2-yl)methyl]-1H- pyrazol-4-yl}-6-phenylfuro[2,3- d]pyrimidine |
| 70 | 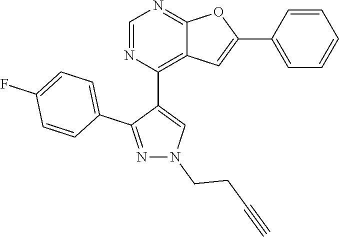 [SMILES: C#CCCn1cc(-c2ncnc3oc(-c4ccccc4)cc23)c(-c2ccc(F)cc2)n1] | 3-[3-(4-fluorophenyl)-4-(6- phenylfuro[2,3-d]pyrimidin-4- yl)-1H-pyrazol-1- yl]propanenitrile |
| 71 | 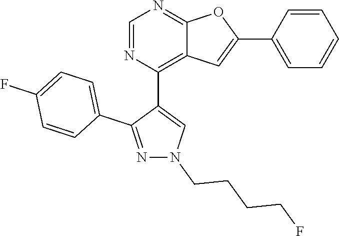 [SMILES: FCCCCn1cc(-c2ncnc3oc(-c4ccccc4)cc23)c(-c2ccc(F)cc2)n1] | 4-[1-(4-fluorobutyl)-3-(4- fluorophenyl)-1H-pyrazol-4-yl]- 6-phenylfuro[2,3-d]pyrimidine |
| 72 | 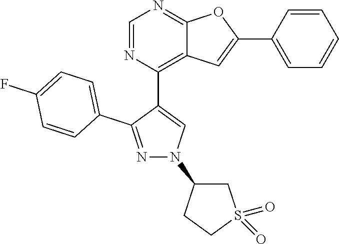 [SMILES: O=S1(=O)CC[C@@H](n2cc(-c3ncnc4oc(-c5ccccc5)cc34)c(-c3ccc(F)cc3)n2)C1] | (3R)-3-[3-(4-fluorophenyl)-4-(6- phenylfuro[2,3-d]pyrimidin-4- yl)-1H-pyrazol-1-yl]-1λ6- thiolane-1,1-dione (absolute stereochemistry arbitrarily assigned) |
| 73 | 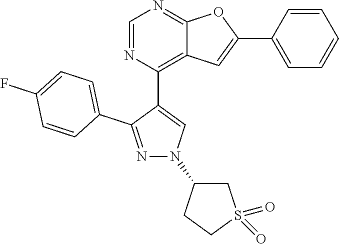 [SMILES: O=S1(=O)CC[C@H](n2cc(-c3ncnc4oc(-c5ccccc5)cc34)c(-c3ccc(F)cc3)n2)C1] | (3S)-3-[3-(4-fluorophenyl)-4-(6- phenylfuro[2,3-d]pyrimidin-4- yl)-1H-pyrazol-1-yl]-1λ6- thiolane-1,1-dione (absolute stereochemistry arbitrarily assigned) |
| 74 | 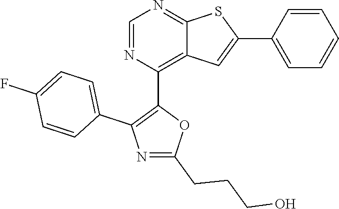 [SMILES: OCCCc1nc(-c2ccc(F)cc2)c(-c2ncnc3sc(-c4ccccc4)cc23)o1] | 3-[4-(4-fluorophenyl)-5-(6- phenylthieno[2,3-d]pyrimidin-4- yl)-1,3-oxazol-2-yl]propan-1-ol |
| 75 | 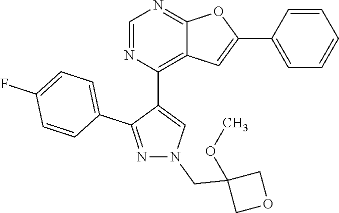 [SMILES: COC1(Cn2cc(-c3ncnc4oc(-c5ccccc5)cc34)c(-c3ccc(F)cc3)n2)COC1] | 4-{3-(4-fluorophenyl)-1-[(3- methoxyoxetan-3-yl)methyl]- 1H-pyrazol-4-yl}-6- phenylfuro[2,3-d]pyrimidine |
| 76 | 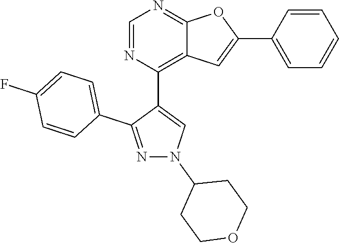 [SMILES: Fc1ccc(-c2nn(C3CCOCC3)cc2-c2ncnc3oc(-c4ccccc4)cc23)cc1] | 4-[3-(4-fluorophenyl)-1-(oxan-4- yl)-1H-pyrazol-4-yl]-6- phenylfuro[2,3-d]pyrimidine |
| 77 | 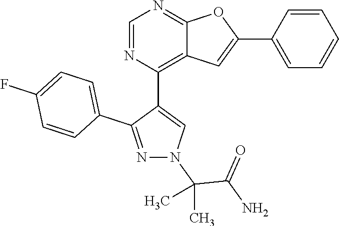 [SMILES: CC(C)(C(N)=O)n1cc(-c2ncnc3oc(-c4ccccc4)cc23)c(-c2ccc(F)cc2)n1] | 2-[3-(4-fluorophenyl)-4-(6- phenylfuro[2,3-d]pyrimidin-4- yl)-1H-pyrazol-1-yl]-2- methylpropanamide |
| 78 | 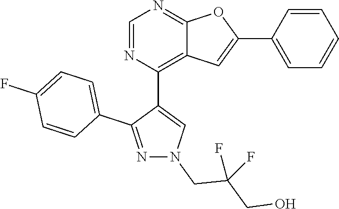 [SMILES: OCC(F)(F)Cn1cc(-c2ncnc3oc(-c4ccccc4)cc23)c(-c2ccc(F)cc2)n1] | 2,2-difluoro-3-[3-(4- fluorophenyl)-4-(6- phenylfuro[2,3-d]pyrimidin-4- yl)-1H-pyrazol-1-yl]propan-1-ol |
| 79 | 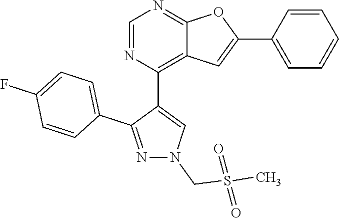 [SMILES: CS(=O)(=O)Cn1cc(-c2ncnc3oc(-c4ccccc4)cc23)c(-c2ccc(F)cc2)n1] | 4-{3-(4-fluorophenyl)-1- [(methanesulfonyl)methyl]-1H- pyrazol-4-yl}-6-phenylfuro[2,3- d]pyrimidine |
| 80 | 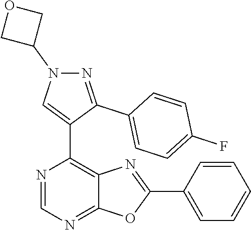 [SMILES: Fc1ccc(-c2nn(C3COC3)cc2-c2ncnc3oc(-c4ccccc4)nc23)cc1] | 7-[3-(4-fluorophenyl)-1-(oxetan- 3-yl)-1H-pyrazol-4-yl]-2- phenyl[1,3]oxazolo[5,4- d]pyrimidine |
| 81 | 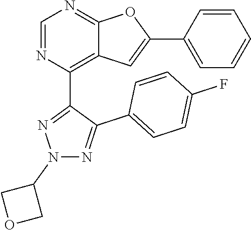 [SMILES: Fc1ccc(-c2nn(C3COC3)nc2-c2ncnc3oc(-c4ccccc4)cc23)cc1] | 4-[5-(4-fluorophenyl)-2-(oxetan- 3-yl)-2H-1,2,3-triazol-4-yl]-6- phenylfuro[2,3-d]pyrimidine |
| 82 | 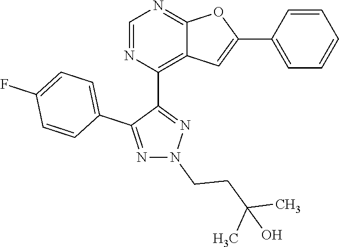 [SMILES: CC(C)(O)CCn1nc(-c2ccc(F)cc2)c(-c2ncnc3oc(-c4ccccc4)cc23)n1] | 4-[4-(4-fluorophenyl)-5-(6- phenylfuro[2,3-d]pyrimidin-4- yl)-2H-1,2,3-triazol-2-yl]-2- methylbutan-2-ol |
| 83 | 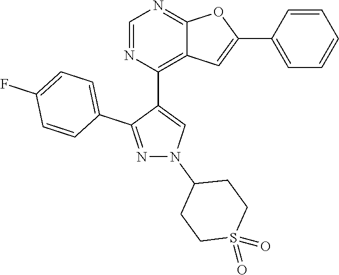 [SMILES: O=S1(=O)CCC(n2cc(-c3ncnc4oc(-c5ccccc5)cc34)c(-c3ccc(F)cc3)n2)CC1] | 4-[3-(4-fluorophenyl)-4-(6- phenylfuro[2,3-d]pyrimidin-4- yl)-1H-pyrazol-1-yl]-1λ6-thiane- 1,1-dione |
| 84 | 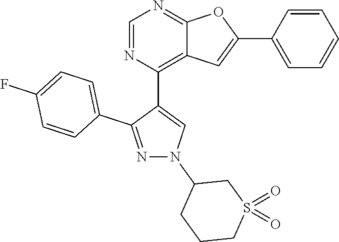 [SMILES: O=S1(=O)CCCC(n2cc(-c3ncnc4oc(-c5ccccc5)cc34)c(-c3ccc(F)cc3)n2)C1] | 3-[3-(4-fluorophenyl)-4-(6- phenylfuro[2,3-d]pyrimidin-4- yl)-1H-pyrazol-1-yl]-1λ6-thiane- 1,1-dione |
| 85 | 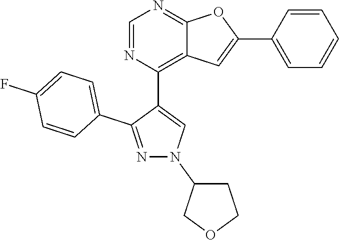 [SMILES: Fc1ccc(-c2nn(C3CCOC3)cc2-c2ncnc3oc(-c4ccccc4)cc23)cc1] | 4-[3-(4-fluorophenyl)-1-(oxolan- 3-yl)-1H-pyrazol-4-yl]-6- phenylfuro[2,3-d]pyrimidine |
| 86 | 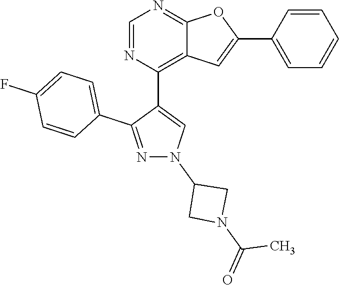 [SMILES: CC(=O)N1CC(n2cc(-c3ncnc4oc(-c5ccccc5)cc34)c(-c3ccc(F)cc3)n2)C1] | 1-{3-[3-(4-fluorophenyl)-4-(6- phenylfuro[2,3-d]pyrimidin-4- yl)-1H-pyrazol-1-yl]azetidin-1- yl}ethan-1-one |
| 87 | 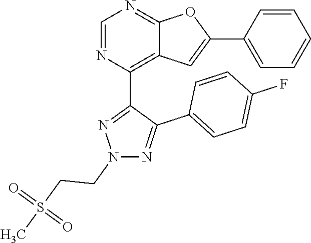 [SMILES: CS(=O)(=O)CCn1nc(-c2ccc(F)cc2)c(-c2ncnc3oc(-c4ccccc4)cc23)n1] | 4-{5-(4-fluorophenyl)-2-[2- (methanesulfonyl)ethyl]-2H- 1,2,3-triazol-4-yl}-6- phenylfuro[2,3-d]pyrimidine |
| 88 | 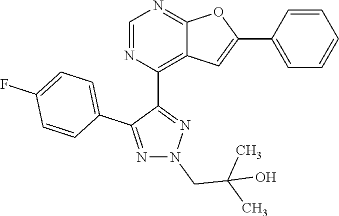 [SMILES: CC(C)(O)Cn1nc(-c2ccc(F)cc2)c(-c2ncnc3oc(-c4ccccc4)cc23)n1] | 1-[4-(4-fluorophenyl)-5-(6- phenylfuro[2,3-d]pyrimidin-4- yl)-2H-1,2,3-triazol-2-yl]-2- methylpropan-2-ol |
| 89 | 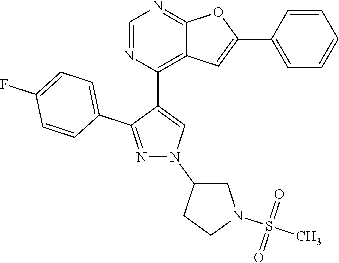 [SMILES: CS(=O)(=O)N1CCC(n2cc(-c3ncnc4oc(-c5ccccc5)cc34)c(-c3ccc(F)cc3)n2)C1] | 4-{3-(4-fluorophenyl)-1-[1- (methanesulfonyl)pyrrolidin-3- yl]-1H-pyrazol-4-yl}-6- phenylfuro[2,3-d]pyrimidine |
| 90 | 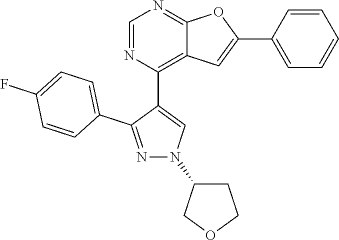 [SMILES: Fc1ccc(-c2nn([C@@H]3CCOC3)cc2-c2ncnc3oc(-c4ccccc4)cc23)cc1] | 4-{3-(4-fluorophenyl)-1-[(3R)- oxolan-3-yl]-1H-pyrazol-4-yl}- 6-phenylfuro[2,3-d]pyrimidine (absolute stereochemistry arbitrarily assigned) |
| 91 | 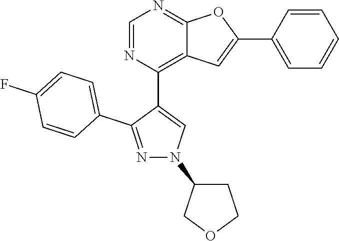 [SMILES: Fc1ccc(-c2nn([C@H]3CCOC3)cc2-c2ncnc3oc(-c4ccccc4)cc23)cc1] | 4-{3-(4-fluorophenyl)-1-[(3S)- oxolan-3-yl]-1H-pyrazol-4-yl}- 6-phenylfuro[2,3-d]pyrimidine (absolute stereochemistry arbitrarily assigned) |
| 92 | 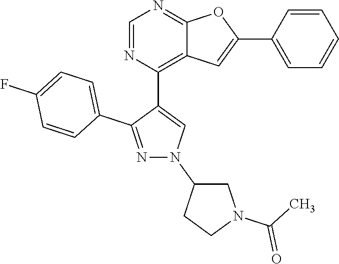 [SMILES: CC(=O)N1CCC(n2cc(-c3ncnc4oc(-c5ccccc5)cc34)c(-c3ccc(F)cc3)n2)C1] | 1-{3-[3-(4-fluorophenyl)-4-(6- phenylfuro[2,3-d]pyrimidin-4- yl)-1H-pyrazol-1-yl]pyrrolidin- 1-yl}ethan-1-one |
| 93 | 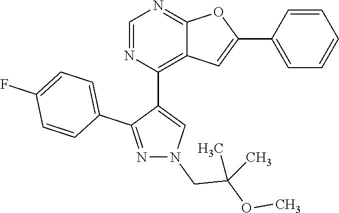 [SMILES: COC(C)(C)Cn1cc(-c2ncnc3oc(-c4ccccc4)cc23)c(-c2ccc(F)cc2)n1] | 4-[3-(4-fluorophenyl)-1-(2- methoxy-2-methylpropyl)-1H- pyrazol-4-yl]-6-phenylfuro[2,3- d]pyrimidine |
| 94 | 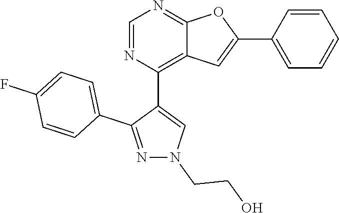 [SMILES: OCCn1cc(-c2ncnc3oc(-c4ccccc4)cc23)c(-c2ccc(F)cc2)n1] | 2-[3-(4-fluorophenyl)-4-(6- phenylfuro[2,3-d]pyrimidin-4- yl)-1H-pyrazol-1-yl]ethan-1-ol |
| 95 | 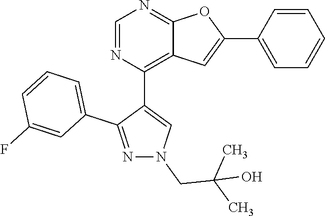 [SMILES: CC(C)(O)Cn1cc(-c2ncnc3oc(-c4ccccc4)cc23)c(-c2cccc(F)c2)n1] | 1-[3-(3-fluorophenyl)-4-(6- phenylfuro[2,3-d]pyrimidin-4- yl)-1H-pyrazol-1-yl]-2- methylpropan-2-ol |
| 96 | 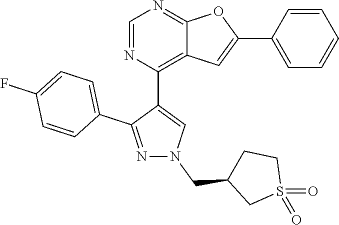 [SMILES: O=S1(=O)CC[C@H](Cn2cc(-c3ncnc4oc(-c5ccccc5)cc34)c(-c3ccc(F)cc3)n2)C1] | (3R)-3-{[3-(4-fluorophenyl)-4- (6-phenylfuro[2,3-d]pyrimidin-4- yl)-1H-pyrazol-1-yl]methyl}- 1λ6-thiolane-1,1-dione (absolute stereochemistry arbitrarily assigned) |
| 97 | 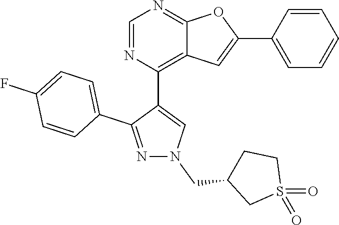 [SMILES: O=S1(=O)CC[C@@H](Cn2cc(-c3ncnc4oc(-c5ccccc5)cc34)c(-c3ccc(F)cc3)n2)C1] | (3S)-3-{[3-(4-fluorophenyl)-4- (6-phenylfuro[2,3-d]pyrimidin-4- yl)-1H-pyrazol-1-yl]methyl}- 1λ6-thiolane-1,1-dione (absolute stereochemistry arbitrarily assigned) |
| 98 | 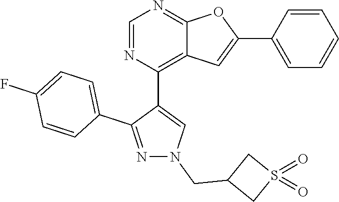 [SMILES: O=S1(=O)CC(Cn2cc(-c3ncnc4oc(-c5ccccc5)cc34)c(-c3ccc(F)cc3)n2)C1] | 3-{[3-(4-fluorophenyl)-4-(6- phenylfuro[2,3-d]pyrimidin-4- yl)-1H-pyrazol-1-yl]methyl}- 1λ6-thietane-1,1-dione |
| 99 | 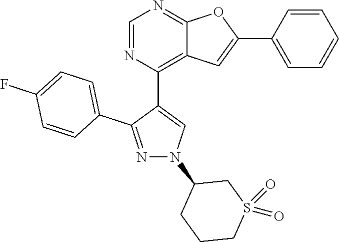 [SMILES: O=S1(=O)CCC[C@@H](n2cc(-c3ncnc4oc(-c5ccccc5)cc34)c(-c3ccc(F)cc3)n2)C1] | (3R)-3-[3-(4-fluorophenyl)-4-(6- phenylfuro[2,3-d]pyrimidin-4- yl)-1H-pyrazol-1-yl]-126-thiane- 1,1-dione (absolute stereochemistry arbitrarily assigned) |
| 100 | 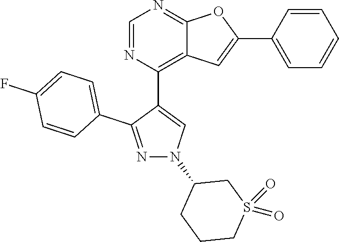 [SMILES: O=S1(=O)CCC[C@H](n2cc(-c3ncnc4oc(-c5ccccc5)cc34)c(-c3ccc(F)cc3)n2)C1] | (3S)-3-[3-(4-fluorophenyl)-4-(6- phenylfuro[2,3-d]pyrimidin-4- yl)-1H-pyrazol-1-yl]-1λ6-thiane- 1,1-dione (absolute stereochemistry arbitrarily assigned) |
| 101 | 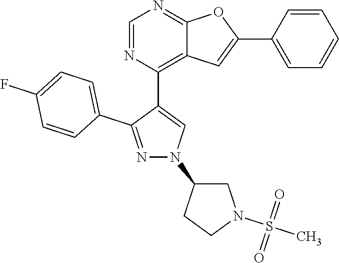 [SMILES: CS(=O)(=O)N1CC[C@@H](n2cc(-c3ncnc4oc(-c5ccccc5)cc34)c(-c3ccc(F)cc3)n2)C1] | 4-{3-(4-fluorophenyl)-1-[(3R)-1- (methanesulfonyl)pyrrolidin-3- yl]-1H-pyrazol-4-yl}-6- phenylfuro[2,3-d]pyrimidine (absolute stereochemistry arbitrarily assigned) |
| 102 | 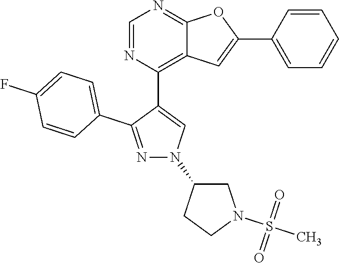 [SMILES: CS(=O)(=O)N1CC[C@H](n2cc(-c3ncnc4oc(-c5ccccc5)cc34)c(-c3ccc(F)cc3)n2)C1] | 4-{3-(4-fluorophenyl)-1-[(3S)-1- (methanesulfonyl)pyrrolidin-3- yl]-1H-pyrazol-4-yl}-6- phenylfuro[2,3-d]pyrimidine (absolute stereochemistry arbitrarily assigned) |
| 103 | 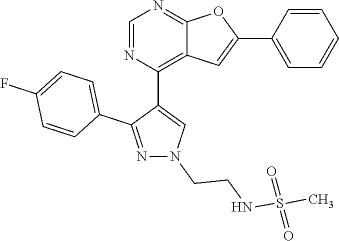 [SMILES: CS(=O)(=O)NCCn1cc(-c2ncnc3oc(-c4ccccc4)cc23)c(-c2ccc(F)cc2)n1] | N-{2-[3-(4-fluorophenyl)-4-(6- phenylfuro[2,3-d]pyrimidin-4- yl)-1H-pyrazol-1- yl]ethyl}methanesulfonamide |
| 104 | 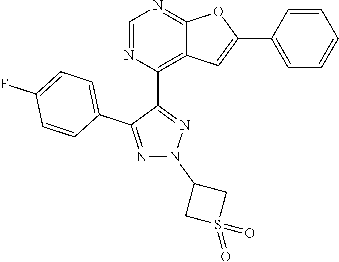 [SMILES: O=S1(=O)CC(n2nc(-c3ccc(F)cc3)c(-c3ncnc4oc(-c5ccccc5)cc34)n2)C1] | 3-[4-(4-fluorophenyl)-5-(6- phenylfuro[2,3-d]pyrimidin-4- yl)-2H-1,2,3-triazol-2-yl]-126- thietane-1,1-dione |
| 105 | 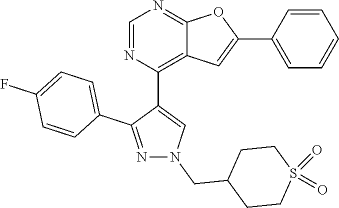 [SMILES: O=S1(=O)CCC(Cn2cc(-c3ncnc4oc(-c5ccccc5)cc34)c(-c3ccc(F)cc3)n2)CC1] | 4-{[3-(4-fluorophenyl)-4-(6- phenylfuro[2,3-d]pyrimidin-4- yl)-1H-pyrazol-1-yl]methyl}- 1λ6-thiane-1,1-dione |
| 106 | 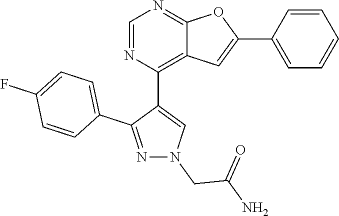 [SMILES: NC(=O)Cn1cc(-c2ncnc3oc(-c4ccccc4)cc23)c(-c2ccc(F)cc2)n1] | 2-[3-(4-fluorophenyl)-4-(6- phenylfuro[2,3-d]pyrimidin-4- yl)-1H-pyrazol-1-yl]acetamide |
| 107 | 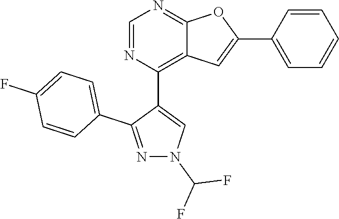 [SMILES: Fc1ccc(-c2nn(C(F)F)cc2-c2ncnc3oc(-c4ccccc4)cc23)cc1] | 4-[1-(difluoromethyl)-3-(4- fluorophenyl)-1H-pyrazol-4-yl]- 6-phenylfuro[2,3-d]pyrimidine |
| 108 | 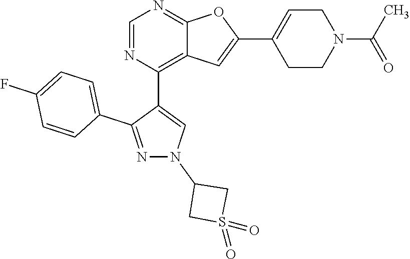 [SMILES: CC(=O)N1CC=C(c2cc3c(-c4cn(C5CS(=O)(=O)C5)nc4-c4ccc(F)cc4)ncnc3o2)CC1] | 3-{4-[6-(1-acetyl-1,2,3,6- tetrahydropyridin-4-yl)furo[2,3- d]pyrimidin-4-yl]-3-(4- fluorophenyl)-1H-pyrazol-1-yl}- 1λ6-thietane-1,1-dione |
| 109 | 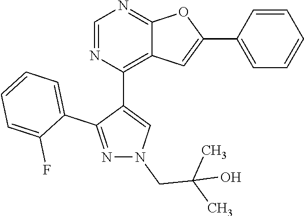 [SMILES: CC(C)(O)Cn1cc(-c2ncnc3oc(-c4ccccc4)cc23)c(-c2ccccc2F)n1] | 1-[3-(2-fluorophenyl)-4-(6- phenylfuro[2,3-d]pyrimidin-4- yl)-1H-pyrazol-1-yl]-2- methylpropan-2-ol |
| 110 | 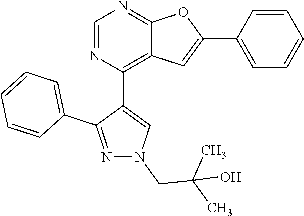 [SMILES: CC(C)(O)Cn1cc(-c2ncnc3oc(-c4ccccc4)cc23)c(-c2ccccc2)n1] | 2-methyl-1-[3-phenyl-4-(6- phenylfuro[2,3-d]pyrimidin-4- yl)-1H-pyrazol-1-yl]propan-2-ol |
| 111 | 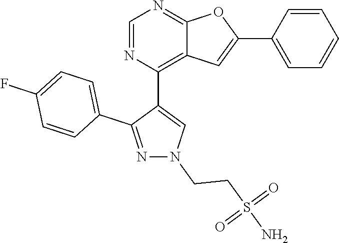 [SMILES: NS(=O)(=O)CCn1cc(-c2ncnc3oc(-c4ccccc4)cc23)c(-c2ccc(F)cc2)n1] | 2-[3-(4-fluorophenyl)-4-(6- phenylfuro[2,3-d]pyrimidin-4- yl)-1H-pyrazol-1-yl]ethane-1- sulfonamide |
| 112 | 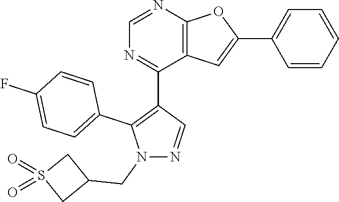 [SMILES: O=S1(=O)CC(Cn2ncc(-c3ncnc4oc(-c5ccccc5)cc34)c2-c2ccc(F)cc2)C1] | 3-((5-(4-fluorophenyl)-4-(6- phenylfuro[2,3-d]pyrimidin-4- yl)-1H-pyrazol-1- yl)methyl)thietane 1,1-dioxide |
| 113 | 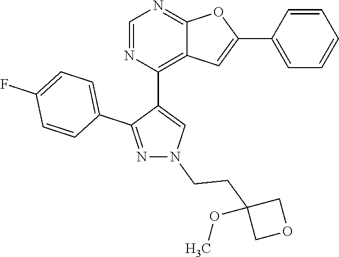 [SMILES: COC1(CCn2cc(-c3ncnc4oc(-c5ccccc5)cc34)c(-c3ccc(F)cc3)n2)COC1] | 4-{3-(4-fluorophenyl)-1-[2-(3- methoxyoxetan-3-yl)ethyl]-1H- pyrazol-4-yl}-6-phenylfuro[2,3- d]pyrimidine |
| 114 | 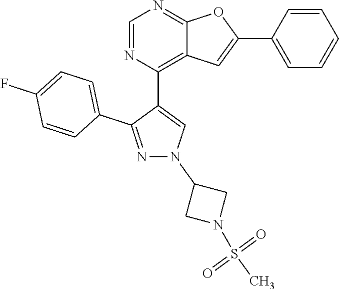 [SMILES: CS(=O)(=O)N1CC(n2cc(-c3ncnc4oc(-c5ccccc5)cc34)c(-c3ccc(F)cc3)n2)C1] | 4-{3-(4-fluorophenyl)-1-[1- (methanesulfonyl)azetidin-3-yl]- 1H-pyrazol-4-yl}-6- phenylfuro[2,3-d]pyrimidine |
| 115 | 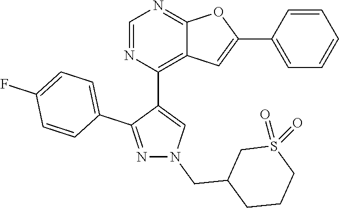 [SMILES: O=S1(=O)CCCC(Cn2cc(-c3ncnc4oc(-c5ccccc5)cc34)c(-c3ccc(F)cc3)n2)C1] | 3-{[3-(4-fluorophenyl)-4-(6- phenylfuro[2,3-d]pyrimidin-4- yl)-1H-pyrazol-1-yl]methyl}- 1λ6-thiane-1,1-dione |
| 116 | 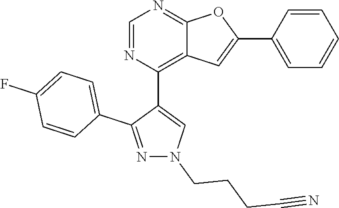 [SMILES: N#CCCCn1cc(-c2ncnc3oc(-c4ccccc4)cc23)c(-c2ccc(F)cc2)n1] | 4-[3-(4-fluorophenyl)-4-(6- phenylfuro[2,3-d]pyrimidin-4- yl)-1H-pyrazol-1-yl]butanenitrile |
| 117 | 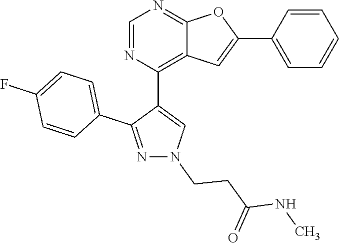 [SMILES: CNC(=O)CCn1cc(-c2ncnc3oc(-c4ccccc4)cc23)c(-c2ccc(F)cc2)n1] | 3-[3-(4-fluorophenyl)-4-(6- phenylfuro[2,3-d]pyrimidin-4- yl)-1H-pyrazol-1-yl]-N- methylpropanamide |
| 118 | 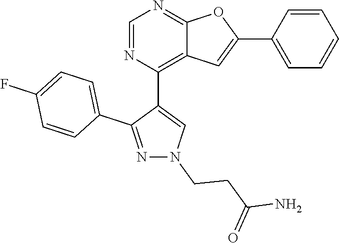 [SMILES: NC(=O)CCn1cc(-c2ncnc3oc(-c4ccccc4)cc23)c(-c2ccc(F)cc2)n1] | 3-[3-(4-fluorophenyl)-4-(6- phenylfuro[2,3-d]pyrimidin-4- yl)-1H-pyrazol-1- yl]propanamide |
| 119 | 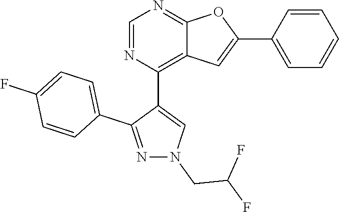 [SMILES: Fc1ccc(-c2nn(CC(F)F)cc2-c2ncnc3oc(-c4ccccc4)cc23)cc1] | 4-[1-(2,2-difluoroethyl)-3-(4- fluorophenyl)-1H-pyrazol-4-yl]- 6-phenylfuro[2,3-d]pyrimidine |
| 120 | 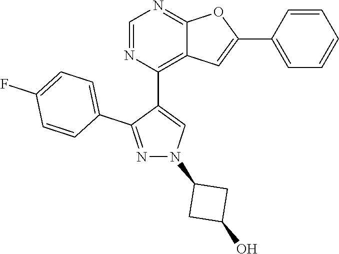 [SMILES: O[C@H]1C[C@@H](n2cc(-c3ncnc4oc(-c5ccccc5)cc34)c(-c3ccc(F)cc3)n2)C1] | (1s,3s)-3-(3-(4-fluorophenyl)-4- (6-phenylfuro[2,3-d]pyrimidin-4- yl)-1H-pyrazol-1-yl)cyclobutan- 1-ol |
| 121 | 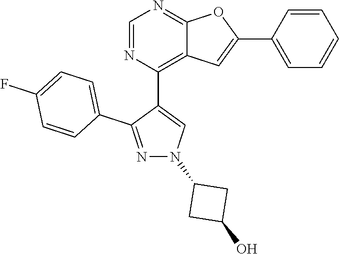 [SMILES: O[C@H]1C[C@H](n2cc(-c3ncnc4oc(-c5ccccc5)cc34)c(-c3ccc(F)cc3)n2)C1] | (1r,3r)-3-(3-(4-fluorophenyl)-4- (6-phenylfuro[2,3-d]pyrimidin-4- yl)-1H-pyrazol-1-yl)cyclobutan- 1-o1 |
| 122 | 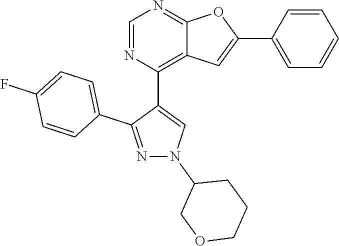 [SMILES: Fc1ccc(-c2nn(C3CCCOC3)cc2-c2ncnc3oc(-c4ccccc4)cc23)cc1] | 4-[3-(4-fluorophenyl)-1-(oxan-3- yl)-1H-pyrazol-4-yl]-6- phenylfuro[2,3-d]pyrimidine |
| 123 | 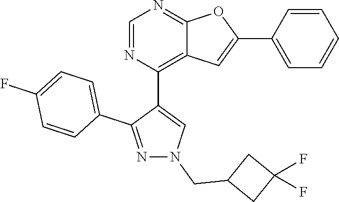 [SMILES: Fc1ccc(-c2nn(CC3CC(F)(F)C3)cc2-c2ncnc3oc(-c4ccccc4)cc23)cc1] | 4-{1-[(3,3- difluorocyclobutyl)methyl]-3-(4- fluorophenyl)-1H-pyrazol-4-yl}- 6-phenylfuro[2,3-d]pyrimidine |
| 124 | 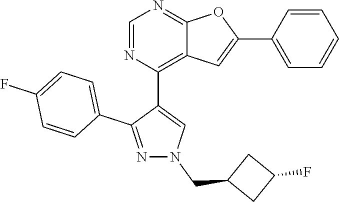 [SMILES: Fc1ccc(-c2nn(C[C@H]3C[C@H](F)C3)cc2-c2ncnc3oc(-c4ccccc4)cc23)cc1] | 4-[1-{[(1r,3r)-3- fluorocyclobutyl]methyl}-3-(4- fluorophenyl)-1H-pyrazol-4-yl]- 6-phenylfuro[2,3-d]pyrimidine |
| 125 | 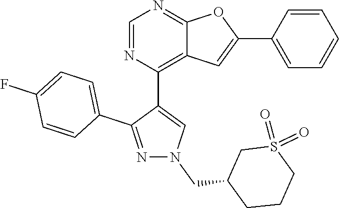 [SMILES: O=S1(=O)CCC[C@H](Cn2cc(-c3ncnc4oc(-c5ccccc5)cc34)c(-c3ccc(F)cc3)n2)C1] | (3R)-3-{[3-(4-fluorophenyl)-4- (6-phenylfuro[2,3-d]pyrimidin-4- yl)-1H-pyrazol-1-yl]methyl}- 1λ6-thiane-1,1-dione (absolute stereochemistry arbitrarily assigned) |
| 126 | 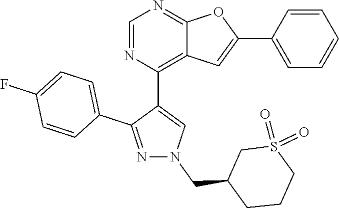 [SMILES: O=S1(=O)CCC[C@@H](Cn2cc(-c3ncnc4oc(-c5ccccc5)cc34)c(-c3ccc(F)cc3)n2)C1] | (3S)-3-{[3-(4-fluorophenyl)-4- (6-phenylfuro[2,3-d]pyrimidin-4- yl)-1H-pyrazol-1-yl]methyl}- 1λ6-thiane-1,1-dione (absolute stereochemistry arbitrarily assigned) |
| 127 | 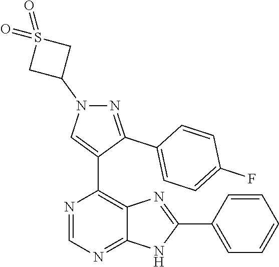 [SMILES: O=S1(=O)CC(n2cc(-c3ncnc4[nH]c(-c5ccccc5)nc34)c(-c3ccc(F)cc3)n2)C1] | 3-[3-(4-fluorophenyl)-4-(8- phenyl-9H-purin-6-y1)-1H- pyrazol-1-yl]-1λ6-thietane-1,1- dione |
| 128 | 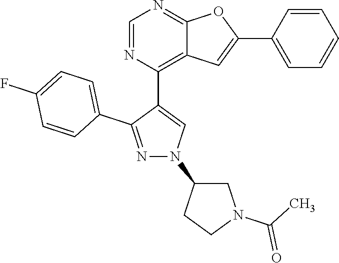 [SMILES: CC(=O)N1CC[C@@H](n2cc(-c3ncnc4oc(-c5ccccc5)cc34)c(-c3ccc(F)cc3)n2)C1] | 1-{(3R)-3-[3-(4-fluorophenyl)-4- (6-phenylfuro[2,3-d]pyrimidin-4- yl)-1H-pyrazol-1-yl]pyrrolidin- 1-yl}ethan-1-one (absolute stereochemistry arbitrarily assigned) |
| 129 | 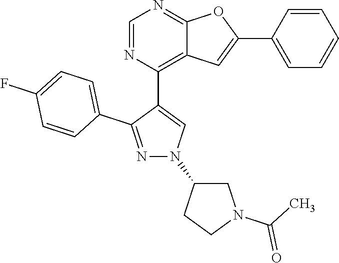 [SMILES: CC(=O)N1CC[C@H](n2cc(-c3ncnc4oc(-c5ccccc5)cc34)c(-c3ccc(F)cc3)n2)C1] | 1-{(3S)-3-[3-(4-fluorophenyl)-4- (6-phenylfuro[2,3-d]pyrimidin-4- yl)-1H-pyrazol-1-yl]pyrrolidin- 1-yl}ethan-1-one (absolute stereochemistry arbitrarily assigned) |
| 130 | 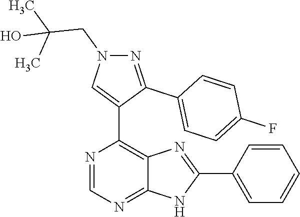 [SMILES: CC(C)(O)Cn1cc(-c2ncnc3[nH]c(-c4ccccc4)nc23)c(-c2ccc(F)cc2)n1] | 1-[3-(4-fluorophenyl)-4-(8- phenyl-9H-purin-6-yl)-1H- pyrazol-1-yl]-2-methylpropan-2- ol |
| 131 | 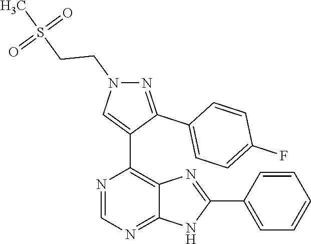 [SMILES: CS(=O)(=O)CCn1cc(-c2ncnc3[nH]c(-c4ccccc4)nc23)c(-c2ccc(F)cc2)n1] | 6-{3-(4-fluorophenyl)-1-[2- (methanesulfonyl)ethyl]-1H- pyrazol-4-yl}-8-phenyl-9H- purine |
| 132 | 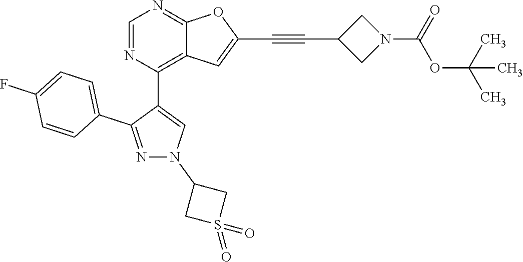 [SMILES: CC(C)(C)OC(=O)N1CC(C#Cc2cc3c(-c4cn(C5CS(=O)(=O)C5)nc4-c4ccc(F)cc4)ncnc3o2)C1] | tert-butyl 3-({4-[1-(1,1-dioxo- 1λ6-thietan-3-y1)-3-(4- fluorophenyl)-1H-pyrazol-4- yl]furo[2,3-d]pyrimidin-6- yl}ethynyl)azetidine-1- carboxylate |
| 133 | 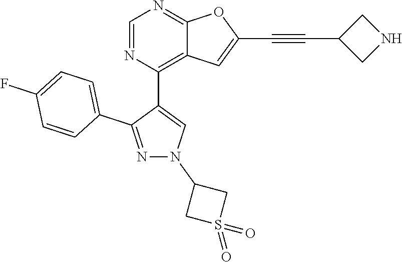 [SMILES: O=S1(=O)CC(n2cc(-c3ncnc4oc(C#CC5CNC5)cc34)c(-c3ccc(F)cc3)n2)C1] | 3-[4-{6-[(azetidin-3- yl)ethynyl]furo[2,3-d]pyrimidin- 4-yl}-3-(4-fluorophenyl)-1H- pyrazol-1-yl]-1λ6-thietane-1,1- dione |
| 134 | 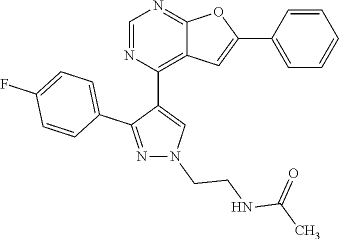 [SMILES: CC(=O)NCCn1cc(-c2ncnc3oc(-c4ccccc4)cc23)c(-c2ccc(F)cc2)n1] | N-{2-[3-(4-fluorophenyl)-4-(6- phenylfuro[2,3-d]pyrimidin-4- yl)-1H-pyrazol-1- yl]ethyl}acetamide |
| 135 | 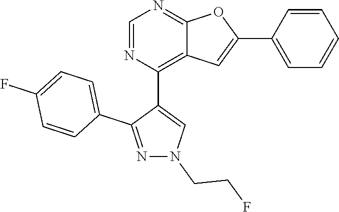 [SMILES: FCCn1cc(-c2ncnc3oc(-c4ccccc4)cc23)c(-c2ccc(F)cc2)n1] | 4-[1-(2-fluoroethyl)-3-(4- fluorophenyl)-1H-pyrazol-4-yl]- 6-phenylfuro[2,3-d]pyrimidine |
| 136 | 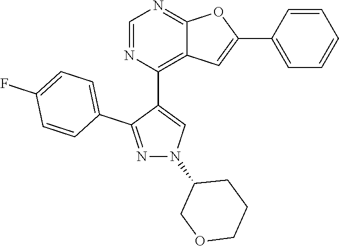 [SMILES: Fc1ccc(-c2nn([C@@H]3CCCOC3)cc2-c2ncnc3oc(-c4ccccc4)cc23)cc1] | 4-{3-(4-fluorophenyl)-1-[(3R)- oxan-3-yl]-1H-pyrazol-4-yl}-6- phenylfuro[2,3-d]pyrimidine (absolute stereochemistry arbitrarily assigned) |
| 137 | 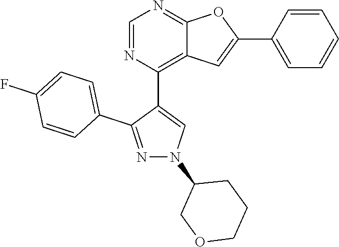 [SMILES: Fc1ccc(-c2nn([C@H]3CCCOC3)cc2-c2ncnc3oc(-c4ccccc4)cc23)cc1] | 4-{3-(4-fluorophenyl)-1-[(3S)- oxan-3-yl]-1H-pyrazol-4-yl}-6- phenylfuro[2,3-d]pyrimidine (absolute stereochemistry arbitrarily assigned) |
| 138 | 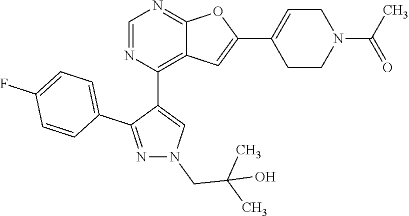 [SMILES: CC(=O)N1CC=C(c2cc3c(-c4cn(CC(C)(C)O)nc4-c4ccc(F)cc4)ncnc3o2)CC1] | 1-[4-{4-[3-(4-fluorophenyl)-1- (2-hydroxy-2-methylpropyl)-1H- pyrazol-4-yl]furo[2,3- d]pyrimidin-6-yl}-3,6- dihydropyridin-1(2H)-yl]ethan- 1-one |
| 139 | 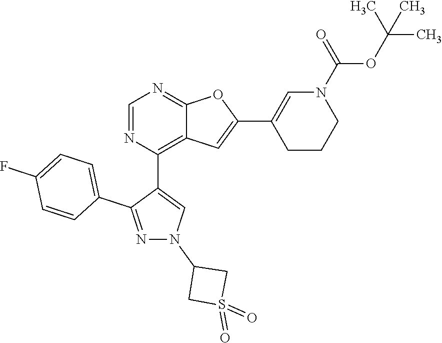 [SMILES: CC(C)(C)OC(=O)N1C=C(c2cc3c(-c4cn(C5CS(=O)(=O)C5)nc4-c4ccc(F)cc4)ncnc3o2)CCC1] | tert-butyl 5-{4-[1-(1,1-dioxo- 1λ6-thietan-3-yl)-3-(4- fluorophenyl)-1H-pyrazol-4- yl]furo[2,3-d]pyrimidin-6-yl}- 3,4-dihydropyridine-1(2H)- carboxylate |
| 140 | 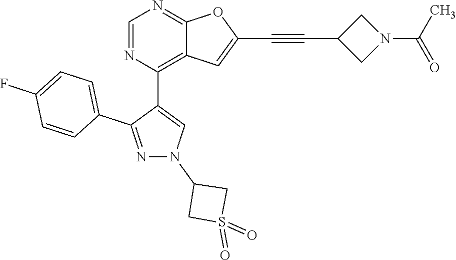 [SMILES: CC(=O)N1CC(C#Cc2cc3c(-c4cn(C5CS(=O)(=O)C5)nc4-c4ccc(F)cc4)ncnc3o2)C1] | 3-[4-{6-[(1-acetylazetidin-3- yl)ethynyl]furo[2,3-d]pyrimidin- 4-yl}-3-(4-fluorophenyl)-1H- pyrazol-1-yl]-126-thietane-1,1- dione |
| 141 | 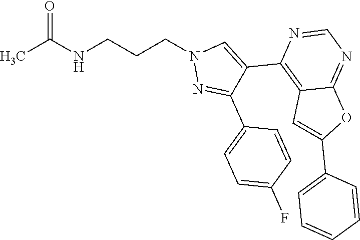 [SMILES: CC(=O)NCCCn1cc(-c2ncnc3oc(-c4ccccc4)cc23)c(-c2ccc(F)cc2)n1] | N-{3-[3-(4-fluorophenyl)-4-(6- phenylfuro[2,3-d]pyrimidin-4- yl)-1H-pyrazol-1- yl]propyl}acetamide |
| 142 | 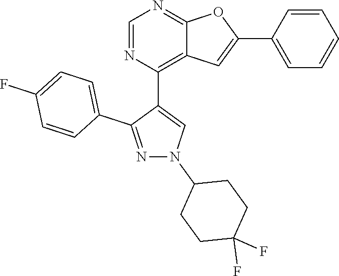 [SMILES: Fc1ccc(-c2nn(C3CCC(F)(F)CC3)cc2-c2ncnc3oc(-c4ccccc4)cc23)cc1] | 4-[1-(4,4-difluorocyclohexyl)-3- (4-fluorophenyl)-1H-pyrazol-4- yl]-6-phenylfuro[2,3- d]pyrimidine |
| 143 | 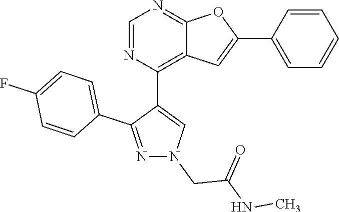 [SMILES: CNC(=O)Cn1cc(-c2ncnc3oc(-c4ccccc4)cc23)c(-c2ccc(F)cc2)n1] | 2-[3-(4-fluorophenyl)-4-(6- phenylfuro[2,3-d]pyrimidin-4- yl)-1H-pyrazol-1-yl]-N- methylacetamide |
| 144 | 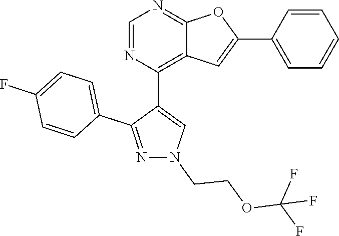 [SMILES: Fc1ccc(-c2nn(CCOC(F)(F)F)cc2-c2ncnc3oc(-c4ccccc4)cc23)cc1] | 4-{3-(4-fluorophenyl)-1-[2- (trifluoromethoxy)ethyl]-1H- pyrazol-4-y1}-6-phenylfuro[2,3- d]pyrimidine |
| 145 | 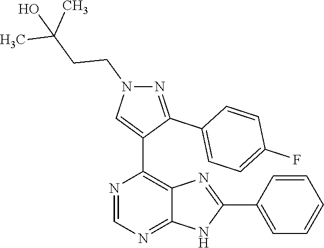 [SMILES: CC(C)(O)CCn1cc(-c2ncnc3[nH]c(-c4ccccc4)nc23)c(-c2ccc(F)cc2)n1] | 4-[3-(4-fluorophenyl)-4-(8- phenyl-9H-purin-6-yl)-1H- pyrazol-1-yl]-2-methylbutan-2-ol |
| 146 | 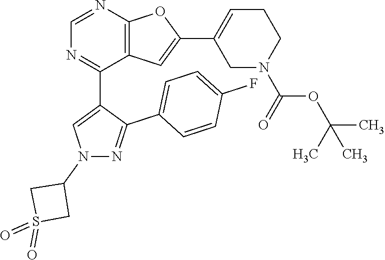 [SMILES: CC(C)(C)OC(=O)N1CCC=C(c2cc3c(-c4cn(C5CS(=O)(=O)C5)nc4-c4ccc(F)cc4)ncnc3o2)C1] | tert-butyl 5-{4-[1-(1,1-dioxo- 1λ6-thietan-3-yl)-3-(4- fluorophenyl)-1H-pyrazol-4- yl]furo[2,3-d]pyrimidin-6-yl}- 3,6-dihydropyridine-1(2H)- carboxylate |
| 147 | 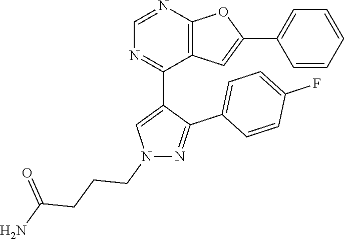 [SMILES: NC(=O)CCCn1cc(-c2ncnc3oc(-c4ccccc4)cc23)c(-c2ccc(F)cc2)n1] | 4-[3-(4-fluorophenyl)-4-(6- phenylfuro[2,3-d]pyrimidin-4- yl)-1H-pyrazol-1-yl]butanamide |
| 148 | 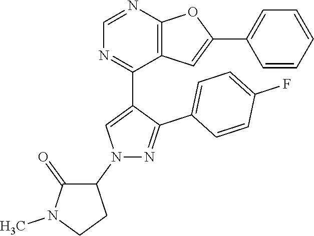 [SMILES: CN1CCC(n2cc(-c3ncnc4oc(-c5ccccc5)cc34)c(-c3ccc(F)cc3)n2)C1=O] | 3-[3-(4-fluorophenyl)-4-(6- phenylfuro[2,3-d]pyrimidin-4- yl)-1H-pyrazol-1-yl]-1- methylpyrrolidin-2-one |
| 149 | 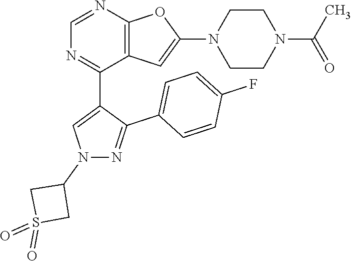 [SMILES: CC(=O)N1CCN(c2cc3c(-c4cn(C5CS(=O)(=O)C5)nc4-c4ccc(F)cc4)ncnc3o2)CC1] | 3-{4-[6-(4-acetylpiperazin-1- yl)furo[2,3-d]pyrimidin-4-yl]-3- (4-fluorophenyl)-1H-pyrazol-1- yl}-126-thietane-1,1-dione |
| 150 | 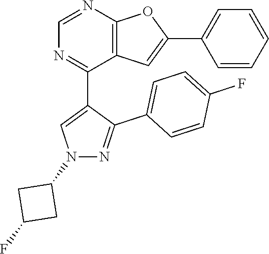 [SMILES: Fc1ccc(-c2nn([C@H]3C[C@@H](F)C3)cc2-c2ncnc3oc(-c4ccccc4)cc23)cc1] | 4-{1-[(1s,3s)-3- fluorocyclobutyl]-3-(4- fluorophenyl)-1H-pyrazol-4-yl}- 6-phenylfuro[2,3-d]pyrimidine |
| 151 | 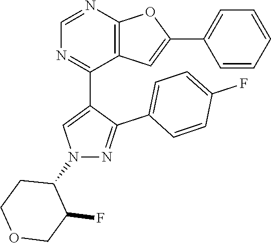 [SMILES: Fc1ccc(-c2nn([C@H]3CCOC[C@@H]3F)cc2-c2ncnc3oc(-c4ccccc4)cc23)cc1] | trans-diastereomer obtained as a racemate of 4-[1-(3-fluorooxan- 4-yl)-3-(4-fluorophenyl)-1H- pyrazol-4-yl]-6-phenylfuro[2,3- d]pyrimidine |
| 152 | 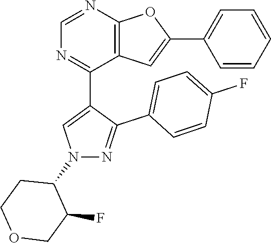 [SMILES: Fc1ccc(-c2nn([C@H]3CCOC[C@@H]3F)cc2-c2ncnc3oc(-c4ccccc4)cc23)cc1] | 4-{1-[(3R,4S)-3-fluorooxan-4- yl]-3-(4-fluorophenyl)-1H- pyrazol-4-yl}-6-phenylfuro[2,3- d]pyrimidine (absolute stereochemistry arbitrarily assigned) |
| 153 | 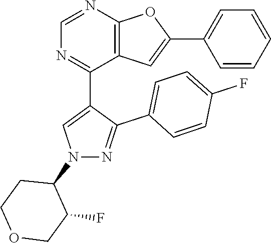 [SMILES: Fc1ccc(-c2nn([C@@H]3CCOC[C@H]3F)cc2-c2ncnc3oc(-c4ccccc4)cc23)cc1] | 4-{1-[(3S,4R)-3-fluorooxan-4- yl]-3-(4-fluorophenyl)-1H- pyrazol-4-yl}-6-phenylfuro[2,3- d]pyrimidine (absolute stereochemistry arbitrarily assigned) |
| 154 | 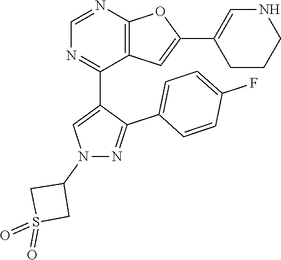 [SMILES: O=S1(=O)CC(n2cc(-c3ncnc4oc(C5=CNCCC5)cc34)c(-c3ccc(F)cc3)n2)C1] | 3-{3-(4-fluorophenyl)-4-[6- (1,4,5,6-tetrahydropyridin-3- yl)furo[2,3-d]pyrimidin-4-yl]- 1H-pyrazol-1-yl}-1λ6-thietane- 1,1-dione |
| 155 | 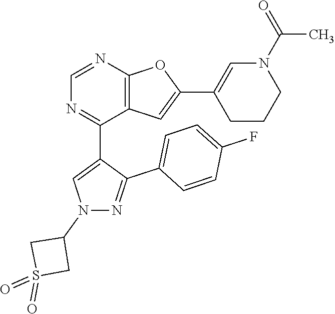 [SMILES: CC(=O)N1C=C(c2cc3c(-c4cn(C5CS(=O)(=O)C5)nc4-c4ccc(F)cc4)ncnc3o2)CCC1] | 3-{4-[6-(1-acetyl-1,4,5,6- tetrahydropyridin-3-yl)furo[2,3- d]pyrimidin-4-yl]-3-(4- fluorophenyl)-1H-pyrazol-1-yl}- 1λ6-thietane-1,1-dione |
| 156 | 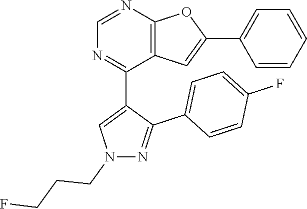 [SMILES: FCCCn1cc(-c2ncnc3oc(-c4ccccc4)cc23)c(-c2ccc(F)cc2)n1] | 4-[3-(4-fluorophenyl)-1-(3- fluoropropyl)-1H-pyrazol-4-yl]- 6-phenylfuro[2,3-d]pyrimidine |
| 157 | 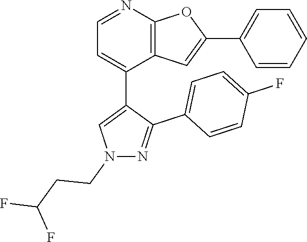 [SMILES: Fc1ccc(-c2nn(CCC(F)F)cc2-c2ccnc3oc(-c4ccccc4)cc23)cc1] | 4-[1-(3,3-difluoropropyl)-3-(4- fluorophenyl)-1H-pyrazol-4-yl]- 2-phenylfuro[2,3-b]pyridine |
| 158 | 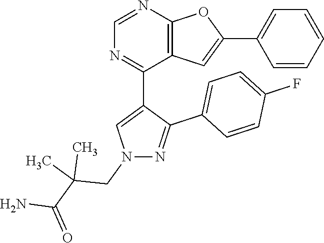 [SMILES: CC(C)(Cn1cc(-c2ncnc3oc(-c4ccccc4)cc23)c(-c2ccc(F)cc2)n1)C(N)=O] | 3-[3-(4-fluorophenyl)-4-(6- phenylfuro[2,3-d]pyrimidin-4- yl)-1H-pyrazol-1-yl]-2,2- dimethylpropanamide |
| 159 | 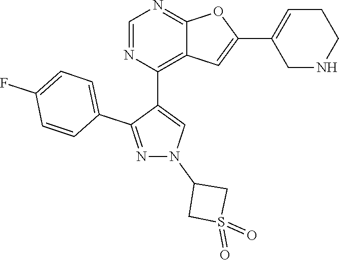 [SMILES: O=S1(=O)CC(n2cc(-c3ncnc4oc(C5=CCCNC5)cc34)c(-c3ccc(F)cc3)n2)C1] | 3-{3-(4-fluorophenyl)-4-[6- (1,2,5,6-tetrahydropyridin-3- yl)furo[2,3-d]pyrimidin-4-yl]- 1H-pyrazol-1-y1}-126-thietane- 1,1-dione |
| 160 | 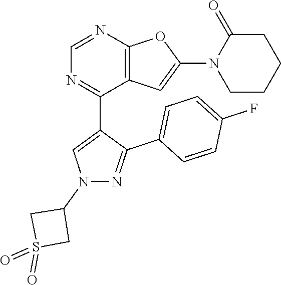 [SMILES: O=C1CCCCN1c1cc2c(-c3cn(C4CS(=O)(=O)C4)nc3-c3ccc(F)cc3)ncnc2o1] | 3-{3-(4-fluorophenyl)-4-[6-(2- oxopiperidin-1-yl)furo[2,3- d]pyrimidin-4-yl]-1H-pyrazol-1- yl}-1λ6-thietane-1,1-dione |
| 161 | 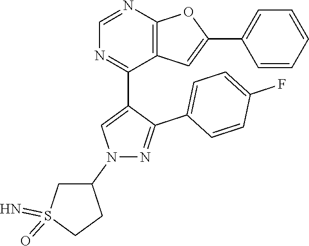 [SMILES: N=S1(=O)CCC(n2cc(-c3ncnc4oc(-c5ccccc5)cc34)c(-c3ccc(F)cc3)n2)C1] | 3-[3-(4-fluorophenyl)-4-(6- phenylfuro[2,3-d]pyrimidin-4- yl)-1H-pyrazol-1-yl]-1-imino- 1λ6-thiolan-1-one |
| 162 | 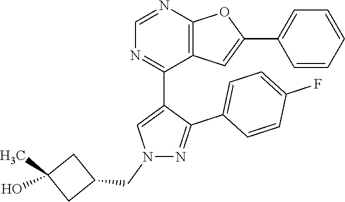 [SMILES: C[C@]1(O)C[C@@H](Cn2cc(-c3ncnc4oc(-c5ccccc5)cc34)c(-c3ccc(F)cc3)n2)C1] | (1s,3s)-3-{[3-(4-fluorophenyl)-4- (6-phenylfuro[2,3-d]pyrimidin-4- yl)-1H-pyrazol-1-yl]methyl}-1- methylcyclobutan-1-ol |
| 163 | 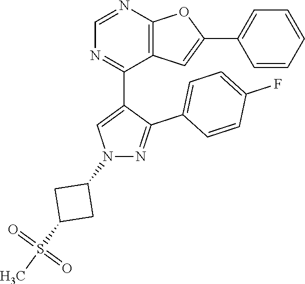 [SMILES: CS(=O)(=O)[C@H]1C[C@@H](n2cc(-c3ncnc4oc(-c5ccccc5)cc34)c(-c3ccc(F)cc3)n2)C1] | 4-{3-(4-fluorophenyl)-1-[(1s,3s)- 3-(methanesulfonyl)cyclobutyl]- 1H-pyrazol-4-yl}-6- phenylfuro[2,3-d]pyrimidine |
| 164 | 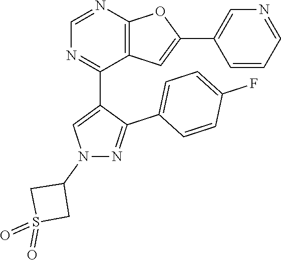 [SMILES: O=S1(=O)CC(n2cc(-c3ncnc4oc(-c5cccnc5)cc34)c(-c3ccc(F)cc3)n2)C1] | 3-{3-(4-fluorophenyl)-4-[6- (pyridin-3-yl)furo[2,3- d]pyrimidin-4-yl]-1H-pyrazol-1- y}-1λ6-thietane-1,1-dione |
| 165 | 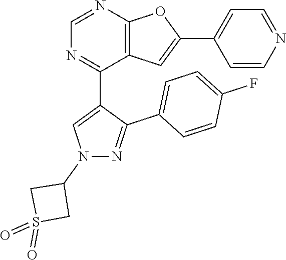 [SMILES: O=S1(=O)CC(n2cc(-c3ncnc4oc(-c5ccncc5)cc34)c(-c3ccc(F)cc3)n2)C1] | 3-{3-(4-fluorophenyl)-4-[6- (pyridin-4-yl)furo[2,3- d]pyrimidin-4-y1]-1H-pyrazol-1- yl}-1λ6-thietane-1,1-dione |
| 166 | 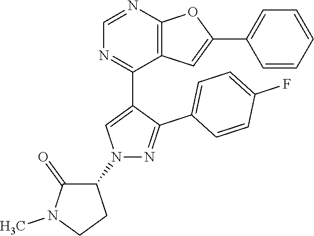 [SMILES: CN1CC[C@@H](n2cc(-c3ncnc4oc(-c5ccccc5)cc34)c(-c3ccc(F)cc3)n2)C1=O] | (3R)-3-[3-(4-fluorophenyl)-4-(6- phenylfuro[2,3-d]pyrimidin-4- yl)-1H-pyrazol-1-yl]-1- methylpyrrolidin-2-one (absolute stereochemistry arbitrarily assigned) |
| 167 | 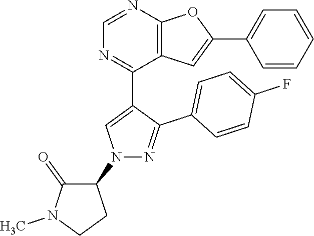 [SMILES: CN1CC[C@H](n2cc(-c3ncnc4oc(-c5ccccc5)cc34)c(-c3ccc(F)cc3)n2)C1=O] | (3S)-3-[3-(4-fluorophenyl)-4-(6- phenylfuro[2,3-d]pyrimidin-4- yl)-1H-pyrazol-1-yl]-1- methylpyrrolidin-2-one (absolute stereochemistry arbitrarily assigned) |
| 168 | 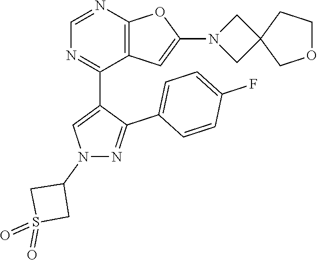 [SMILES: O=S1(=O)CC(n2cc(-c3ncnc4oc(N5CC6(CCOC6)C5)cc34)c(-c3ccc(F)cc3)n2)C1] | 3-{3-(4-fluorophenyl)-4-[6-(6- oxa-2-azaspiro[3.4]octan-2- yl)furo[2,3-d]pyrimidin-4-yl]- 1H-pyrazol-1-yl}-1λ6-thietane- 1,1-dione |
| 169 | 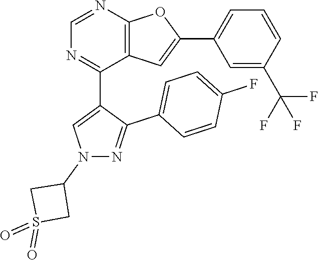 [SMILES: O=S1(=O)CC(n2cc(-c3ncnc4oc(-c5cccc(C(F)(F)F)c5)cc34)c(-c3ccc(F)cc3)n2)C1] | 3-[3-(4-fluorophenyl)-4-{6-[3- (trifluoromethyl)phenyl]furo[2,3- d]pyrimidin-4-yl}-1H-pyrazol-1- yl]-1λ6-thietane-1,1-dione |
| 170 | 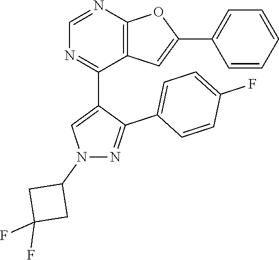 [SMILES: Fc1ccc(-c2nn(C3CC(F)(F)C3)cc2-c2ncnc3oc(-c4ccccc4)cc23)cc1] | 4-[1-(3,3-difluorocyclobutyl)-3- (4-fluorophenyl)-1H-pyrazol-4- yl]-6-phenylfuro[2,3- d]pyrimidine |
| 171 | 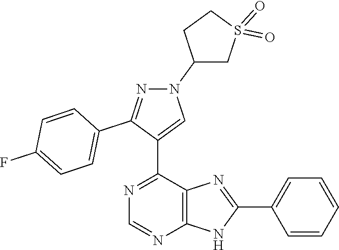 [SMILES: O=S1(=O)CCC(n2cc(-c3ncnc4[nH]c(-c5ccccc5)nc34)c(-c3ccc(F)cc3)n2)C1] | 3-[3-(4-fluorophenyl)-4-(8- phenyl-9H-purin-6-yl)-1H- pyrazol-1-yl]-1λ6-thiolane-1,1- dione |
| 172 | 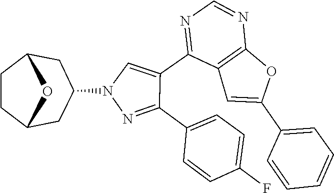 [SMILES: Fc1ccc(-c2nn([C@H]3C[C@H]4CC[C@@H](C3)O4)cc2-c2ncnc3oc(-c4ccccc4)cc23)cc1] | 4-{3-(4-fluorophenyl)-1- [(1R,3r,5S)-8- oxabicyclo[3.2.1]octan-3-yl]-1H- pyrazol-4-yl}-6-phenylfuro[2,3- d]pyrimidine |
| 173 | 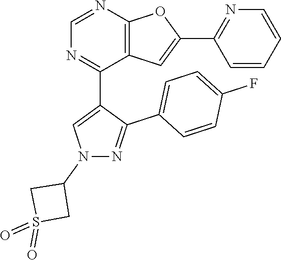 [SMILES: O=S1(=O)CC(n2cc(-c3ncnc4oc(-c5ccccn5)cc34)c(-c3ccc(F)cc3)n2)C1] | 3-{3-(4-fluorophenyl)-4-[6- (pyridin-2-yl)furo[2,3- d]pyrimidin-4-yl]-1H-pyrazol-1- yl}-126-thietane-1,1-dione |
| 174 | 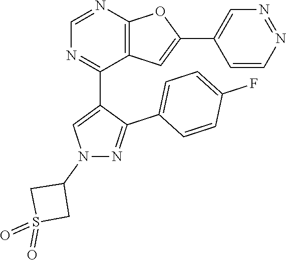 [SMILES: O=S1(=O)CC(n2cc(-c3ncnc4oc(-c5ccnnc5)cc34)c(-c3ccc(F)cc3)n2)C1] | 3-{3-(4-fluorophenyl)-4-[6- (pyridazin-4-yl)furo[2,3- d]pyrimidin-4-yl]-1H-pyrazol-1- yl}-1λ6-thietane-1,1-dione |
| 175 | 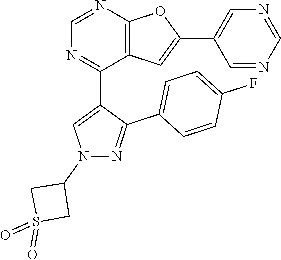 [SMILES: O=S1(=O)CC(n2cc(-c3ncnc4oc(-c5cncnc5)cc34)c(-c3ccc(F)cc3)n2)C1] | 3-{3-(4-fluorophenyl)-4-[6- (pyrimidin-5-y1)furo[2,3- d]pyrimidin-4-yl]-1H-pyrazol-1- yl}-1λ6-thietane-1,1-dione |
| 176 | 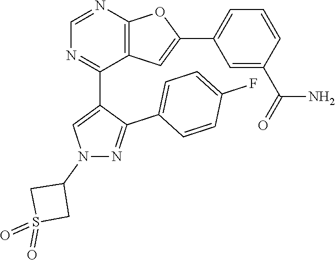 [SMILES: NC(=O)c1cccc(-c2cc3c(-c4cn(C5CS(=O)(=O)C5)nc4-c4ccc(F)cc4)ncnc3o2)c1] | 3-{4-[1-(1,1-dioxo-1λ6-thietan- 3-yl)-3-(4-fluorophenyl)-1H- pyrazol-4-yl]furo[2,3- d]pyrimidin-6-yl}benzamide |
| 177 | 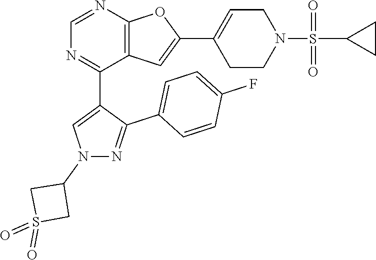 [SMILES: O=S1(=O)CC(n2cc(-c3ncnc4oc(C5=CCN(S(=O)(=O)C6CC6)CC5)cc34)c(-c3ccc(F)cc3)n2)C1] | 3-[4-{6-[1- (cyclopropanesulfonyl)-1,2,3,6- tetrahydropyridin-4-yl]furo[2,3- d]pyrimidin-4-yl}-3-(4- fluorophenyl)-1H-pyrazol-1-yl]- 1λ6-thietane-1,1-dione |
| 178 | 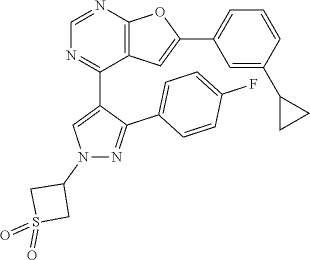 [SMILES: O=S1(=O)CC(n2cc(-c3ncnc4oc(-c5cccc(C6CC6)c5)cc34)c(-c3ccc(F)cc3)n2)C1] | 3-{4-[6-(3- cyclopropylphenyl)furo[2,3- d]pyrimidin-4-yl]-3-(4- fluorophenyl)-1H-pyrazol-1-yl}- 1λ6-thietane-1,1-dione |
| 179 | 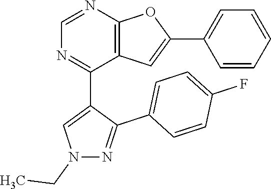 [SMILES: CCn1cc(-c2ncnc3oc(-c4ccccc4)cc23)c(-c2ccc(F)cc2)n1] | 4-[1-ethyl-3-(4-fluorophenyl)- 1H-pyrazol-4-yl]-6- phenylfuro[2,3-d]pyrimidine |
| 180 | 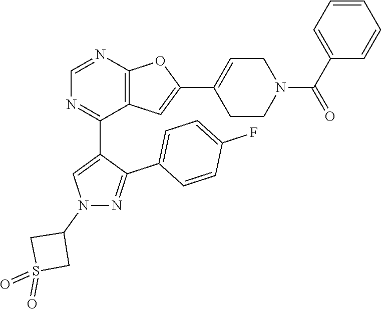 [SMILES: O=C(c1ccccc1)N1CC=C(c2cc3c(-c4cn(C5CS(=O)(=O)C5)nc4-c4ccc(F)cc4)ncnc3o2)CC1] | 3-{4-[6-(1-benzoyl-1,2,3,6- tetrahydropyridin-4-yl)furo[2,3- d]pyrimidin-4-y1]-3-(4- fluorophenyl)-1H-pyrazol-1-yl}- 1λ6-thietane-1,1-dione |
| 181 | 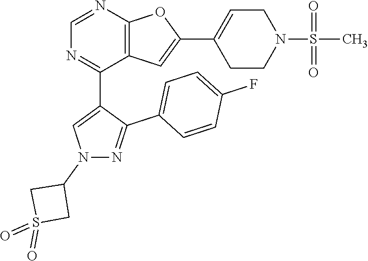 [SMILES: CS(=O)(=O)N1CC=C(c2cc3c(-c4cn(C5CS(=O)(=O)C5)nc4-c4ccc(F)cc4)ncnc3o2)CC1] | 3-[3-(4-fluorophenyl)-4-{6-[1- (methanesulfonyl)-1,2,3,6- tetrahydropyridin-4-yl]furo[2,3- d]pyrimidin-4-yl}-1H-pyrazol-1- yl]-1λ6-thietane-1,1-dione |
| 182 | 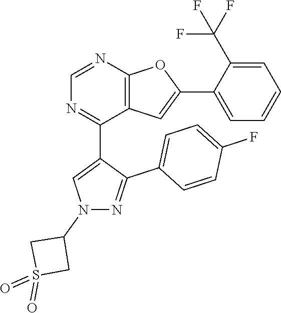 [SMILES: O=S1(=O)CC(n2cc(-c3ncnc4oc(-c5ccccc5C(F)(F)F)cc34)c(-c3ccc(F)cc3)n2)C1] | 3-[3-(4-fluorophenyl)-4-{6-[2- (trifluoromethyl)phenyl]furo[2,3- d]pyrimidin-4-yl}-1H-pyrazol-1- yl]-1λ6-thietane-1,1-dione |
| 183 | 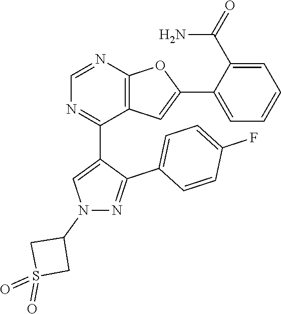 [SMILES: NC(=O)c1ccccc1-c1cc2c(-c3cn(C4CS(=O)(=O)C4)nc3-c3ccc(F)cc3)ncnc2o1] | 2-{4-[1-(1,1-dioxo-1λ6-thietan- 3-yl)-3-(4-fluorophenyl)-1H- pyrazol-4-yl]furo[2,3- d]pyrimidin-6-yl}benzamide |
| 184 | 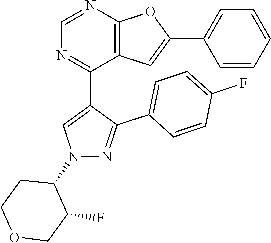 [SMILES: Fc1ccc(-c2nn([C@H]3CCOC[C@H]3F)cc2-c2ncnc3oc(-c4ccccc4)cc23)cc1] | cis-diastereomer obtained as a racemate of 4-[1-(3-fluorooxan- 4-yl)-3-(4-fluorophenyl)-1H- pyrazol-4-yl]-6-phenylfuro[2,3- d]pyrimidine |
| 185 | 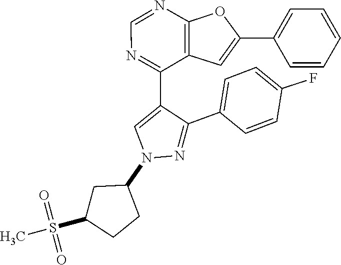 [SMILES: CS(=O)(=O)[C@@H]1CC[C@H](n2cc(-c3ncnc4oc(-c5ccccc5)cc34)c(-c3ccc(F)cc3)n2)C1] | cis-diastereomer obtained as a racemate of 4-{3-(4- fluorophenyl)-1-[3- (methanesulfonyl)cyclopentyl]- 1H-pyrazol-4-y1}-6- phenylfuro[2,3-d]pyrimidine |
| 186 | 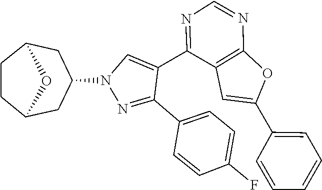 [SMILES: Fc1ccc(-c2nn([C@@H]3C[C@H]4CC[C@@H](C3)O4)cc2-c2ncnc3oc(-c4ccccc4)cc23)cc1] | 4-{3-(4-fluorophenyl)-1- [(1R,3s,5S)-8- oxabicyclo[3.2.1]octan-3-yl]-1H- pyrazol-4-yl}-6-phenylfuro[2,3- d]pyrimidine |
| 187 | 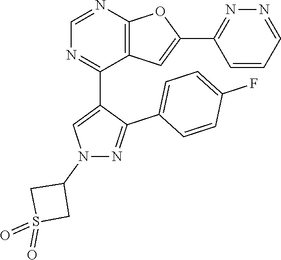 [SMILES: O=S1(=O)CC(n2cc(-c3ncnc4oc(-c5cccnn5)cc34)c(-c3ccc(F)cc3)n2)C1] | 3-{3-(4-fluorophenyl)-4-[6- (pyridazin-3-y1)furo[2,3- d]pyrimidin-4-yl]-1H-pyrazol-1- yl}-1λ6-thietane-1,1-dione |
| 188 | 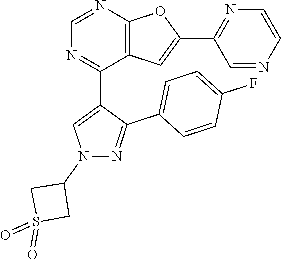 [SMILES: O=S1(=O)CC(n2cc(-c3ncnc4oc(-c5cnccn5)cc34)c(-c3ccc(F)cc3)n2)C1] | 3-{3-(4-fluorophenyl)-4-[6- (pyrazin-2-y1)furo[2,3- d]pyrimidin-4-yl]-1H-pyrazol-1- yl}-1λ6-thietane-1,1-dione |
| 189 | 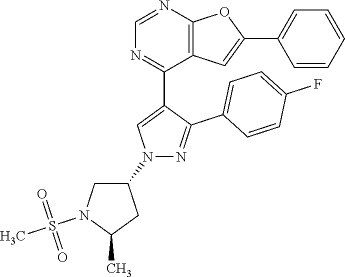 [SMILES: C[C@@H]1C[C@@H](n2cc(-c3ncnc4oc(-c5ccccc5)cc34)c(-c3ccc(F)cc3)n2)CN1S(C)(=O)=O] | 4-{3-(4-fluorophenyl)-1- [(3R,5R)-1-(methanesulfonyl)-5- methylpyrrolidin-3-yl]-1H- pyrazol-4-yl}-6-phenylfuro[2,3- d]pyrimidine |
| 190 | 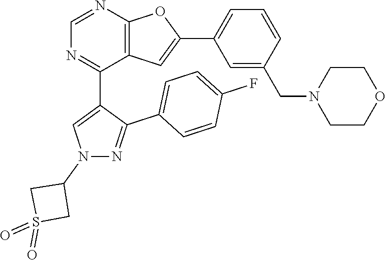 [SMILES: O=S1(=O)CC(n2cc(-c3ncnc4oc(-c5cccc(CN6CCOCC6)c5)cc34)c(-c3ccc(F)cc3)n2)C1] | 3-[3-(4-fluorophenyl)-4-(6-{3- [(morpholin-4- yl)methyl]phenyl}furo[2,3- d]pyrimidin-4-yl)-1H-pyrazol-1- yl]-126-thietane-1,1-dione |
| 191 | 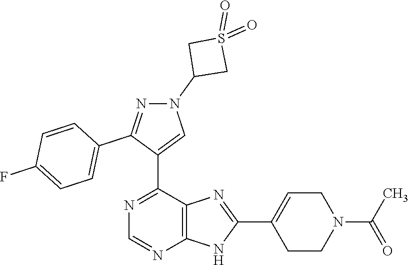 [SMILES: CC(=O)N1CC=C(c2nc3c(-c4cn(C5CS(=O)(=O)C5)nc4-c4ccc(F)cc4)ncnc3[nH]2)CC1] | 3-{4-[8-(1-acetyl-1,2,3,6- tetrahydropyridin-4-yl)-9H- purin-6-y1]-3-(4-fluorophenyl)- 1H-pyrazol-1-yl}-126-thietane- 1,1-dione |
| 192 | 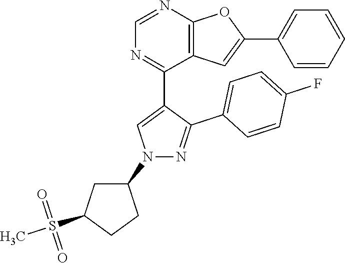 [SMILES: CS(=O)(=O)[C@@H]1CC[C@H](n2cc(-c3ncnc4oc(-c5ccccc5)cc34)c(-c3ccc(F)cc3)n2)C1] | 4-{3-(4-fluorophenyl)-1- [(1S,3R)-3- (methanesulfonyl)cyclopenty1]- 1H-pyrazol-4-yl}-6- phenylfuro[2,3-d]pyrimidine (absolute stereochemistry arbitrarily assigned) |
| 193 | 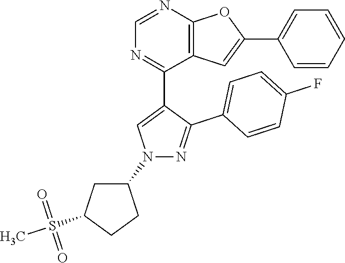 [SMILES: CS(=O)(=O)[C@H]1CC[C@@H](n2cc(-c3ncnc4oc(-c5ccccc5)cc34)c(-c3ccc(F)cc3)n2)C1] | 4-{3-(4-fluorophenyl)-1- [(1R,3S)-3- (methanesulfonyl)cyclopentyl]- 1H-pyrazol-4-yl}-6- phenylfuro[2,3-d]pyrimidine (absolute stereochemistry arbitrarily assigned) |
| 194 | 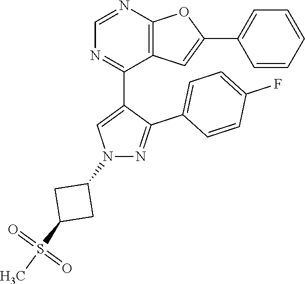 [SMILES: CS(=O)(=O)[C@H]1C[C@H](n2cc(-c3ncnc4oc(-c5ccccc5)cc34)c(-c3ccc(F)cc3)n2)C1] | 4-{3-(4-fluorophenyl)-1-[(1r,3r)- 3-(methanesulfonyl)cyclobutyl]- 1H-pyrazol-4-yl}-6- phenylfuro[2,3-d]pyrimidine |
| 195 | 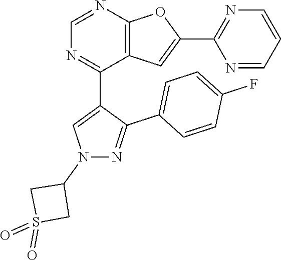 [SMILES: O=S1(=O)CC(n2cc(-c3ncnc4oc(-c5ncccn5)cc34)c(-c3ccc(F)cc3)n2)C1] | 3-{3-(4-fluorophenyl)-4-[6- (pyrimidin-2-y1)furo[2,3- d]pyrimidin-4-y1]-1H-pyrazol-1- yl}-1λ6-thietane-1,1-dione |
| 196 | 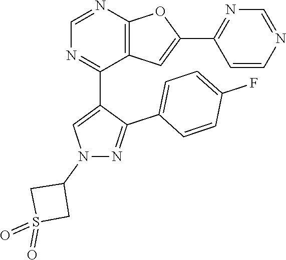 [SMILES: O=S1(=O)CC(n2cc(-c3ncnc4oc(-c5ccncn5)cc34)c(-c3ccc(F)cc3)n2)C1] | 3-{3-(4-fluorophenyl)-4-[6- (pyrimidin-4-yl)furo[2,3- d]pyrimidin-4-y1]-1H-pyrazol-1- yl}-126-thietane-1,1-dione |
| 197 | 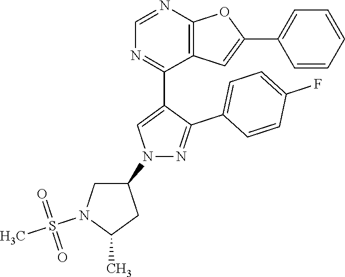 [SMILES: C[C@H]1C[C@H](n2cc(-c3ncnc4oc(-c5ccccc5)cc34)c(-c3ccc(F)cc3)n2)CN1S(C)(=O)=O] | 4-{3-(4-fluorophenyl)-1- [(3S,5S)-1-(methanesulfonyl)-5- methylpyrrolidin-3-yl]-1H- pyrazol-4-yl}-6-phenylfuro[2,3- d]pyrimidine |
| 198 | 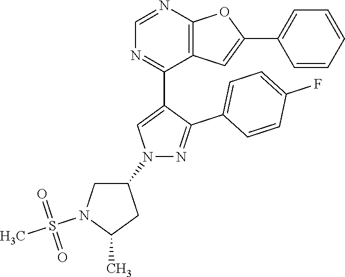 [SMILES: C[C@H]1C[C@@H](n2cc(-c3ncnc4oc(-c5ccccc5)cc34)c(-c3ccc(F)cc3)n2)CN1S(C)(=O)=O] | 4-{3-(4-fluorophenyl)-1- [(3R,5S)-1-(methanesulfonyl)-5- methylpyrrolidin-3-y1]-1H- pyrazol-4-yl}-6-phenylfuro[2,3- d]pyrimidine |
| 199 | 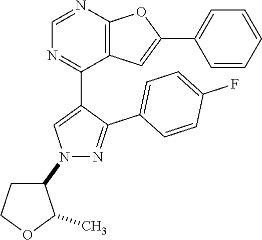 [SMILES: C[C@@H]1OCC[C@H]1n1cc(-c2ncnc3oc(-c4ccccc4)cc23)c(-c2ccc(F)cc2)n1] | trans-diastereomer obtained as a racemate of 2,5-anhydro-1,3,4- trideoxy-3-[3-(4-fluorophenyl)- 4-(6-phenylfuro[2,3-d]pyrimidin- 4-yl)-1H-pyrazol-1-yl]pentitol |
| 200 | 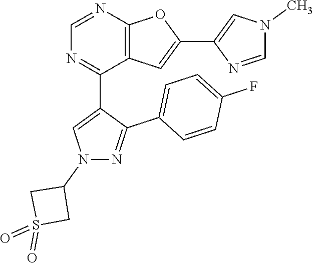 [SMILES: Cn1cnc(-c2cc3c(-c4cn(C5CS(=O)(=O)C5)nc4-c4ccc(F)cc4)ncnc3o2)c1] | 3-{3-(4-fluorophenyl)-4-[6-(1- methyl-1H-imidazol-4- yl)furo[2,3-d]pyrimidin-4-yl]- 1H-pyrazol-1-yl}-126-thietane- 1,1-dione |
| 201 | 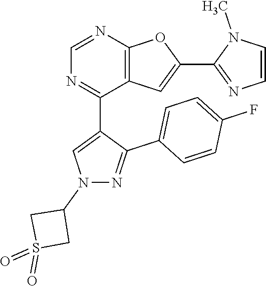 [SMILES: Cn1ccnc1-c1cc2c(-c3cn(C4CS(=O)(=O)C4)nc3-c3ccc(F)cc3)ncnc2o1] | 3-{3-(4-fluorophenyl)-4-[6-(1- methyl-1H-imidazol-2- yl)furo[2,3-d]pyrimidin-4-yl]- 1H-pyrazol-1-yl}-126-thietane- 1,1-dione |
| 202 | 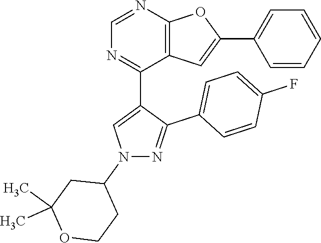 [SMILES: CC1(C)CC(n2cc(-c3ncnc4oc(-c5ccccc5)cc34)c(-c3ccc(F)cc3)n2)CCO1] | 4-[1-(2,2-dimethyloxan-4-yl)-3- (4-fluorophenyl)-1H-pyrazol-4- yl]-6-phenylfuro[2,3- d]pyrimidine |
| 203 | 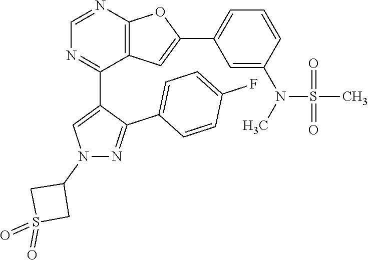 [SMILES: CN(c1cccc(-c2cc3c(-c4cn(C5CS(=O)(=O)C5)nc4-c4ccc(F)cc4)ncnc3o2)c1)S(C)(=O)=O] | N-(3-{4-[1-(1,1-dioxo-126- thietan-3-yl)-3-(4-fluorophenyl)- 1H-pyrazol-4-yl]furo[2,3- d]pyrimidin-6-yl}phenyl)-N- methylmethanesulfonamide |
| 204 | 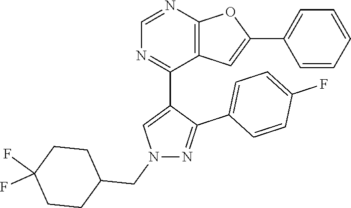 [SMILES: Fc1ccc(-c2nn(CC3CCC(F)(F)CC3)cc2-c2ncnc3oc(-c4ccccc4)cc23)cc1] | 4-{1-[(4,4- difluorocyclohexyl)methyl]-3-(4- fluorophenyl)-1H-pyrazol-4-yl}- 6-phenylfuro[2,3-d]pyrimidine |
| 205 | 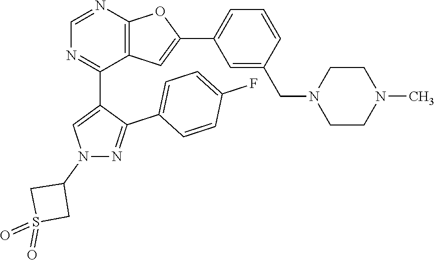 [SMILES: CN1CCN(Cc2cccc(-c3cc4c(-c5cn(C6CS(=O)(=O)C6)nc5-c5ccc(F)cc5)ncnc4o3)c2)CC1] | 3-[3-(4-fluorophenyl)-4-(6-{3- [(4-methylpiperazin-1- yl)methyl]phenyl}furo[2,3- d]pyrimidin-4-yl)-1H-pyrazol-1- yl]-1λ6-thietane-1,1-dione |
| 206 | 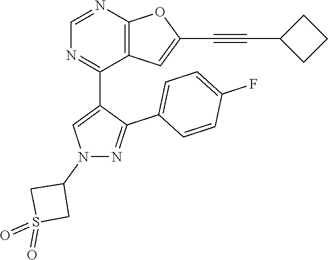 [SMILES: O=S1(=O)CC(n2cc(-c3ncnc4oc(C#CC5CCC5)cc34)c(-c3ccc(F)cc3)n2)C1] | 3-{4-[6- (cyclobutylethynyl)furo[2,3- d]pyrimidin-4-y1]-3-(4- fluorophenyl)-1H-pyrazol-1-yl}- 1λ6-thietane-1,1-dione |
| 207 | 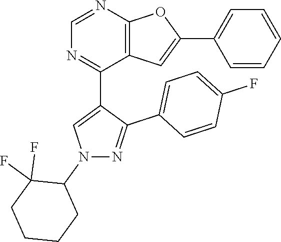 [SMILES: Fc1ccc(-c2nn(C3CCCCC3(F)F)cc2-c2ncnc3oc(-c4ccccc4)cc23)cc1] | 4-[1-(2,2-difluorocyclohexyl)-3- (4-fluorophenyl)-1H-pyrazol-4- yl]-6-phenylfuro[2,3- d]pyrimidine |
| 208 | 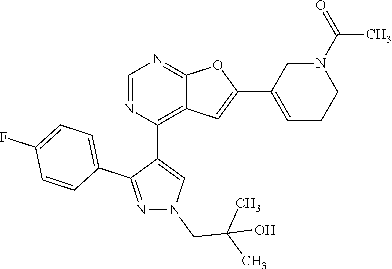 [SMILES: CC(=O)N1CCC=C(c2cc3c(-c4cn(CC(C)(C)O)nc4-c4ccc(F)cc4)ncnc3o2)C1] | 1-[5-{4-[3-(4-fluorophenyl)-1- (2-hydroxy-2-methylpropyl)-1H- pyrazol-4-yl]furo[2,3- d]pyrimidin-6-yl}-3,6- dihydropyridin-1(2H)-yl]ethan- 1-one |
| 209 | 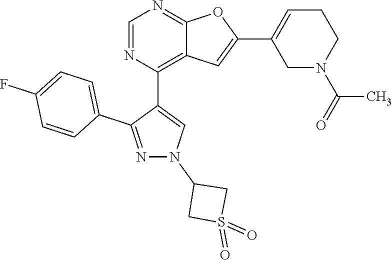 [SMILES: CC(=O)N1CCC=C(c2cc3c(-c4cn(C5CS(=O)(=O)C5)nc4-c4ccc(F)cc4)ncnc3o2)C1] | 3-{4-[6-(1-acetyl-1,2,5,6- tetrahydropyridin-3-yl)furo[2,3- d]pyrimidin-4-yl]-3-(4- fluorophenyl)-1H-pyrazol-1-yl}- 1λ6-thietane-1,1-dione |
| 210 | 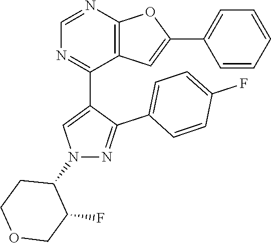 [SMILES: Fc1ccc(-c2nn([C@H]3CCOC[C@H]3F)cc2-c2ncnc3oc(-c4ccccc4)cc23)cc1] | 4-{1-[(3S,4S)-3-fluorooxan-4- yl]-3-(4-fluorophenyl)-1H- pyrazol-4-yl}-6-phenylfuro[2,3- d]pyrimidine (absolute stereochemistry arbitrarily assigned) |
| 211 | 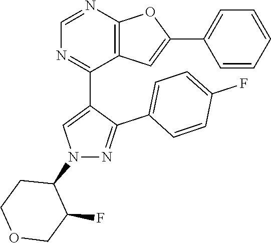 [SMILES: Fc1ccc(-c2nn([C@@H]3CCOC[C@@H]3F)cc2-c2ncnc3oc(-c4ccccc4)cc23)cc1] | 4-{1-[(3R,4R)-3-fluorooxan-4- yl]-3-(4-fluorophenyl)-1H- pyrazol-4-yl}-6-phenylfuro[2,3- d]pyrimidine (absolute stereochemistry arbitrarily assigned) |
| 212 | 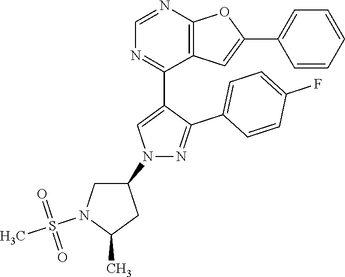 [SMILES: C[C@@H]1C[C@H](n2cc(-c3ncnc4oc(-c5ccccc5)cc34)c(-c3ccc(F)cc3)n2)CN1S(C)(=O)=O] | 4-{3-(4-fluorophenyl)-1- [(3S,5R)-1-(methanesulfonyl)-5- methylpyrrolidin-3-yl]-1H- pyrazol-4-yl}-6-phenylfuro[2,3- d]pyrimidine |
| 213 | 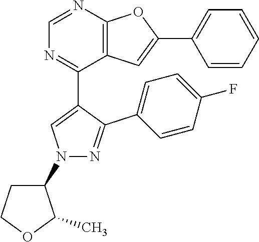 [SMILES: C[C@@H]1OCC[C@H]1n1cc(-c2ncnc3oc(-c4ccccc4)cc23)c(-c2ccc(F)cc2)n1] | 4-(3-(4-fluorophenyl)-1- ((2S,3R)-2- methyltetrahydrofuran-3-yl)-1H- pyrazol-4-yl)-6-phenylfuro[2,3- d]pyrimidine (absolute stereochemistry arbitrarily assigned) |
| 214 | 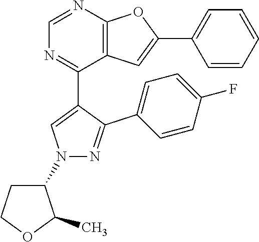 [SMILES: C[C@H]1OCC[C@@H]1n1cc(-c2ncnc3oc(-c4ccccc4)cc23)c(-c2ccc(F)cc2)n1] | 4-(3-(4-fluorophenyl)-1- ((2R,3S)-2- methyltetrahydrofuran-3-yl)-1H- pyrazol-4-yl)-6-phenylfuro[2,3- d]pyrimidine (absolute stereochemistry arbitrarily assigned) |
| 215 | 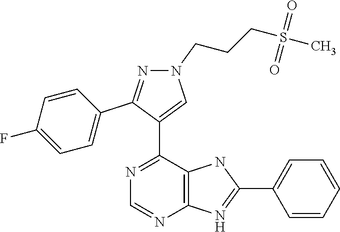 [SMILES: CS(=O)(=O)CCCn1cc(-c2ncnc3nc(-c4ccccc4)[nH]c23)c(-c2ccc(F)cc2)n1] | 6-{3-(4-fluorophenyl)-1-[3- (methanesulfonyl)propyl]-1H- pyrazol-4-yl}-8-phenyl-9H- purine |
| 216 | 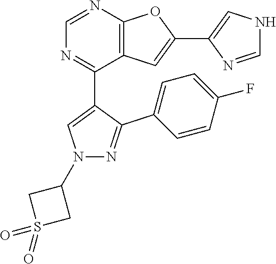 [SMILES: O=S1(=O)CC(n2cc(-c3ncnc4oc(-c5c[nH]cn5)cc34)c(-c3ccc(F)cc3)n2)C1] | 3-{3-(4-fluorophenyl)-4-[6-(1H- imidazol-4-yl)furo[2,3- d]pyrimidin-4-yl]-1H-pyrazol-1- yl}-1λ6-thietane-1,1-dione |
| 217 | 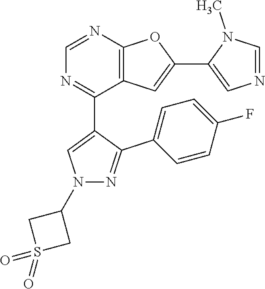 [SMILES: Cn1cncc1-c1cc2c(-c3cn(C4CS(=O)(=O)C4)nc3-c3ccc(F)cc3)ncnc2o1] | 3-{3-(4-fluorophenyl)-4-[6-(1- methyl-1H-imidazol-5- yl)furo[2,3-d]pyrimidin-4-yl]- 1H-pyrazol-1-yl}-1λ6-thietane- 1,1-dione |
| 218 | 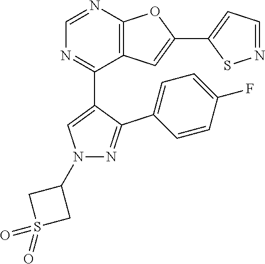 [SMILES: O=S1(=O)CC(n2cc(-c3ncnc4oc(-c5ccns5)cc34)c(-c3ccc(F)cc3)n2)C1] | 3-{3-(4-fluorophenyl)-4-[6-(1,2- thiazol-5-yl)furo[2,3- d]pyrimidin-4-yl]-1H-pyrazol-1- yl}-1λ6-thietane-1,1-dione |
| 219 | 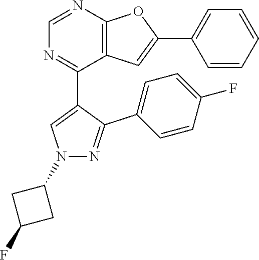 [SMILES: Fc1ccc(-c2nn([C@H]3C[C@H](F)C3)cc2-c2ncnc3oc(-c4ccccc4)cc23)cc1] | 4-{1-[(1r,3r)-3- fluorocyclobutyl]-3-(4- fluorophenyl)-1H-pyrazol-4-yl}- 6-phenylfuro[2,3-d]pyrimidine |
| 220 | 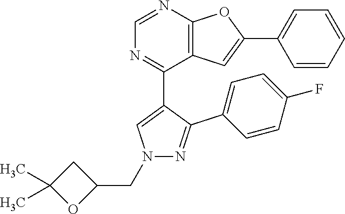 [SMILES: CC1(C)CC(Cn2cc(-c3ncnc4oc(-c5ccccc5)cc34)c(-c3ccc(F)cc3)n2)O1] | 4-{1-[(4,4-dimethyloxetan-2- yl)methyl]-3-(4-fluorophenyl)- 1H-pyrazol-4-yl}-6- phenylfuro[2,3-d]pyrimidine |
| 221 | 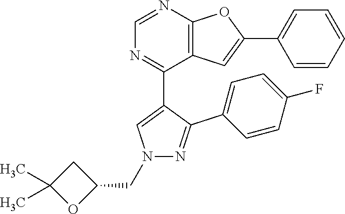 [SMILES: CC1(C)C[C@H](Cn2cc(-c3ncnc4oc(-c5ccccc5)cc34)c(-c3ccc(F)cc3)n2)O1] | 4-[1-{[(2R)-4,4-dimethyloxetan- 2-yl]methyl}-3-(4-fluorophenyl)- 1H-pyrazol-4-yl]-6- phenylfuro[2,3-d]pyrimidine (absolute stereochemistry arbitrarily assigned) |
| 222 | 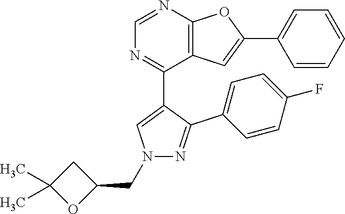 [SMILES: CC1(C)C[C@@H](Cn2cc(-c3ncnc4oc(-c5ccccc5)cc34)c(-c3ccc(F)cc3)n2)O1] | 4-[1-{[(2S)-4,4-dimethyloxetan- 2-yl]methyl}-3-(4-fluorophenyl)- 1H-pyrazol-4-yl]-6- phenylfuro[2,3-d]pyrimidine (absolute stereochemistry arbitrarily assigned) |
| 223 | 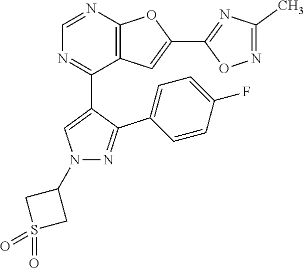 [SMILES: Cc1noc(-c2cc3c(-c4cn(C5CS(=O)(=O)C5)nc4-c4ccc(F)cc4)ncnc3o2)n1] | 3-{3-(4-fluorophenyl)-4-[6-(3- methyl-1,2,4-oxadiazol-5- yl)furo[2,3-d]pyrimidin-4-yl]- 1H-pyrazol-1-yl}-126-thietane- 1,1-dione |
| 224 | 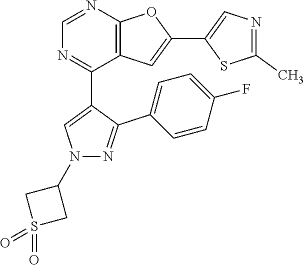 [SMILES: Cc1ncc(-c2cc3c(-c4cn(C5CS(=O)(=O)C5)nc4-c4ccc(F)cc4)ncnc3o2)s1] | 3-{3-(4-fluorophenyl)-4-[6-(2- methyl-1,3-thiazol-5-yl)furo[2,3- d]pyrimidin-4-yl]-1H-pyrazol-1- yl}-1λ6-thietane-1,1-dione |
| 225 | 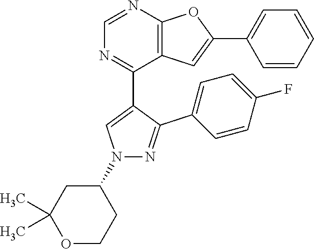 [SMILES: CC1(C)C[C@H](n2cc(-c3ncnc4oc(-c5ccccc5)cc34)c(-c3ccc(F)cc3)n2)CCO1] | 4-{1-[(4R)-2,2-dimethyloxan-4- yl]-3-(4-fluorophenyl)-1H- pyrazol-4-yl}-6-phenylfuro[2,3- d]pyrimidine (absolute stereochemistry arbitrarily assigned) |
| 226 | 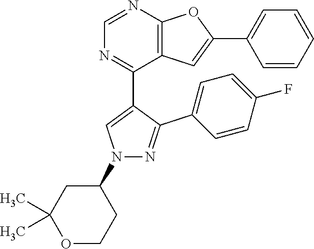 [SMILES: CC1(C)C[C@@H](n2cc(-c3ncnc4oc(-c5ccccc5)cc34)c(-c3ccc(F)cc3)n2)CCO1] | 4-{1-[(4S)-2,2-dimethyloxan-4- yl]-3-(4-fluorophenyl)-1H- pyrazol-4-yl}-6-phenylfuro[2,3- d]pyrimidine (absolute stereochemistry arbitrarily assigned) |
| 227 | 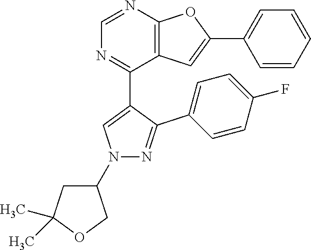 [SMILES: CC1(C)CC(n2cc(-c3ncnc4oc(-c5ccccc5)cc34)c(-c3ccc(F)cc3)n2)CO1] | 4-[1-(5,5-dimethyloxolan-3-yl)- 3-(4-fluorophenyl)-1H-pyrazol- 4-yl]-6-phenylfuro[2,3- d]pyrimidine |
| 228 | 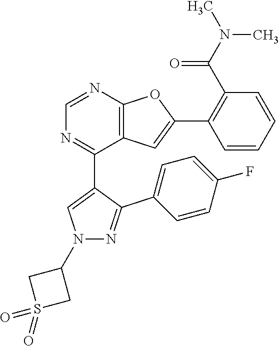 [SMILES: CN(C)C(=O)c1ccccc1-c1cc2c(-c3cn(C4CS(=O)(=O)C4)nc3-c3ccc(F)cc3)ncnc2o1] | 2-{4-[1-(1, 1-dioxo-1λ6-thietan- 3-yl)-3-(4-fluorophenyl)-1H- pyrazol-4-yl]furo[2,3- d]pyrimidin-6-yl}-N,N- dimethylbenzamide |
| 229 | 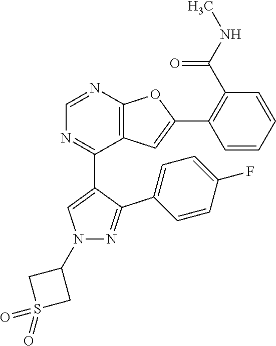 [SMILES: CNC(=O)c1ccccc1-c1cc2c(-c3cn(C4CS(=O)(=O)C4)nc3-c3ccc(F)cc3)ncnc2o1] | 2-{4-[1-(1, 1-dioxo-1λ6-thietan- 3-yl)-3-(4-fluorophenyl)-1H- pyrazol-4-yl]furo[2,3- d]pyrimidin-6-yl}-N- methylbenzamide |
| 230 | 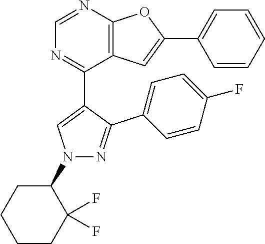 [SMILES: Fc1ccc(-c2nn([C@@H]3CCCCC3(F)F)cc2-c2ncnc3oc(-c4ccccc4)cc23)cc1] | 4-{1-[(1R)-2,2- difluorocyclohexyl]-3-(4- fluorophenyl)-1H-pyrazol-4-yl}- 6-phenylfuro[2,3-d]pyrimidine (absolute stereochemistry arbitrarily assigned) |
| 231 | 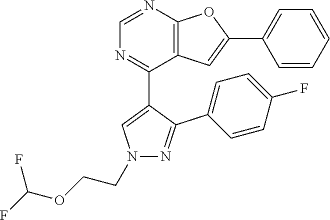 [SMILES: Fc1ccc(-c2nn(CCOC(F)F)cc2-c2ncnc3oc(-c4ccccc4)cc23)cc1] | 4-{1-[2-(difluoromethoxy)ethyl]- 3-(4-fluorophenyl)-1H-pyrazol- 4-yl}-6-phenylfuro[2,3- d]pyrimidine |
| 232 | 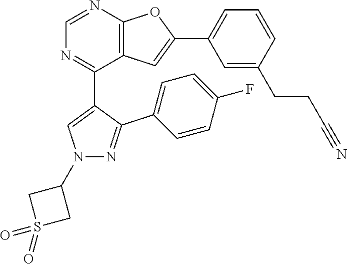 [SMILES: N#CCCc1cccc(-c2cc3c(-c4cn(C5CS(=O)(=O)C5)nc4-c4ccc(F)cc4)ncnc3o2)c1] | 3-(3-{4-[1-(1,1-dioxo-1λ6- thietan-3-yl)-3-(4-fluorophenyl)- 1H-pyrazol-4-yl]furo[2,3- d]pyrimidin-6- yl}phenyl)propanenitrile |
| 233 | 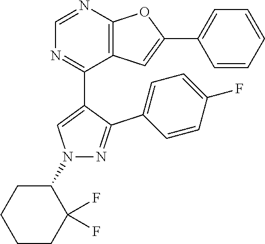 [SMILES: Fc1ccc(-c2nn([C@H]3CCCCC3(F)F)cc2-c2ncnc3oc(-c4ccccc4)cc23)cc1] | 4-{1-[(1S)-2,2- difluorocyclohexyl]-3-(4- fluorophenyl)-1H-pyrazol-4-yl}- 6-phenylfuro[2,3-d]pyrimidine (absolute stereochemistry arbitrarily assigned) |
| 234 | 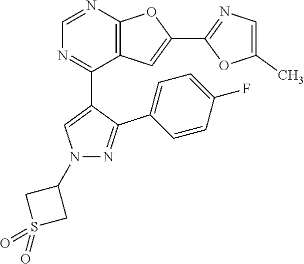 [SMILES: Cc1cnc(-c2cc3c(-c4cn(C5CS(=O)(=O)C5)nc4-c4ccc(F)cc4)ncnc3o2)o1] | 3-{3-(4-fluorophenyl)-4-[6-(5- methyl-1,3-oxazol-2-yl)furo[2,3- d]pyrimidin-4-yl]-1H-pyrazol-1- yl}-1λ6-thietane-1,1-dione |
| 235 | 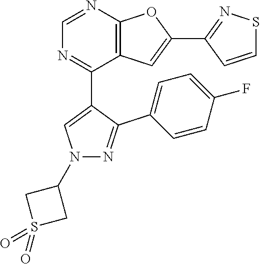 [SMILES: O=S1(=O)CC(n2cc(-c3ncnc4oc(-c5ccsn5)cc34)c(-c3ccc(F)cc3)n2)C1] | 3-{3-(4-fluorophenyl)-4-[6-(1,2- thiazol-3-yl)furo[2,3- d]pyrimidin-4-yl]-1H-pyrazol-1- yl}-1λ6-thietane-1,1-dione |
| 236 | 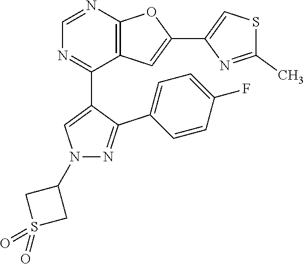 [SMILES: Cc1nc(-c2cc3c(-c4cn(C5CS(=O)(=O)C5)nc4-c4ccc(F)cc4)ncnc3o2)cs1] | 3-{3-(4-fluorophenyl)-4-[6-(2- methyl-1,3-thiazol-4-yl)furo[2,3- d]pyrimidin-4-yl]-1H-pyrazol-1- yl}-1λ6-thietane-1,1-dione |
| 237 | 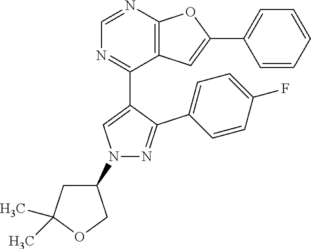 [SMILES: CC1(C)C[C@@H](n2cc(-c3ncnc4oc(-c5ccccc5)cc34)c(-c3ccc(F)cc3)n2)CO1] | 4-{1-[(3R)-5,5-dimethyloxolan- 3-yl]-3-(4-fluorophenyl)-1H- pyrazol-4-yl}-6-phenylfuro[2,3- d]pyrimidine (absolute stereochemistry arbitrarily assigned) |
| 238 | 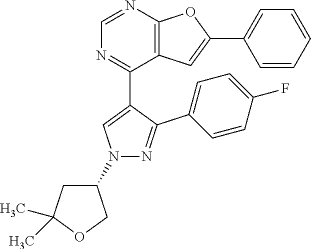 [SMILES: CC1(C)C[C@H](n2cc(-c3ncnc4oc(-c5ccccc5)cc34)c(-c3ccc(F)cc3)n2)CO1] | 4-{1-[(3S)-5,5-dimethyloxolan- 3-yl]-3-(4-fluorophenyl)-1H- pyrazol-4-yl}-6-phenylfuro[2,3- d]pyrimidine (absolute stereochemistry arbitrarily assigned) |
| 239 | 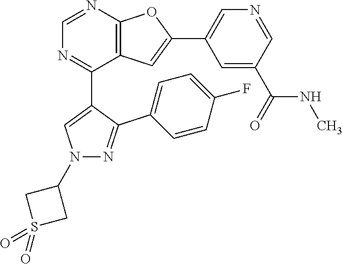 [SMILES: CNC(=O)c1cncc(-c2cc3c(-c4cn(C5CS(=O)(=O)C5)nc4-c4ccc(F)cc4)ncnc3o2)c1] | 5-{4-[1-(1,1-dioxo-1λ6-thietan- 3-yl)-3-(4-fluorophenyl)-1H- pyrazol-4-yl]furo[2,3- d]pyrimidin-6-yl}-N- methylpyridine-3-carboxamide |
| 240 | 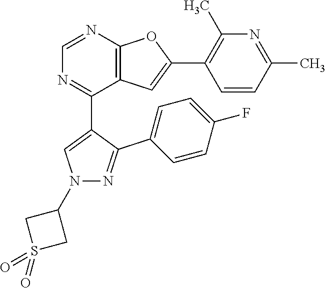 [SMILES: Cc1ccc(-c2cc3c(-c4cn(C5CS(=O)(=O)C5)nc4-c4ccc(F)cc4)ncnc3o2)c(C)n1] | 3-{4-[6-(2,6-dimethylpyridin-3- yl)furo[2,3-d]pyrimidin-4-yl]-3- (4-fluorophenyl)-1H-pyrazol-1- yl}-1λ6-thietane-1,1-dione |
| 241 | 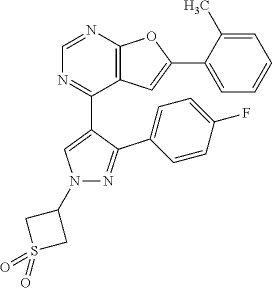 [SMILES: Cc1ccccc1-c1cc2c(-c3cn(C4CS(=O)(=O)C4)nc3-c3ccc(F)cc3)ncnc2o1] | 3-{3-(4-fluorophenyl)-4-[6-(3- methylpyridin-2-yl)furo[2,3- d]pyrimidin-4-yl]-1H-pyrazol-1- yl}-126-thietane-1,1-dione |
| 242 | 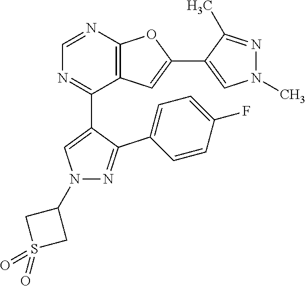 [SMILES: Cc1nn(C)cc1-c1cc2c(-c3cn(C4CS(=O)(=O)C4)nc3-c3ccc(F)cc3)ncnc2o1] | 3-{4-[6-(1,3-dimethyl-1H- pyrazol-4-yl)furo[2,3- d]pyrimidin-4-yl]-3-(4- fluorophenyl)-1H-pyrazol-1-yl}- 1λ6-thietane-1,1-dione |
| 243 | 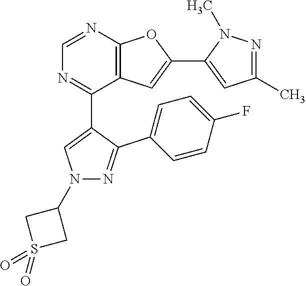 [SMILES: Cc1cc(-c2cc3c(-c4cn(C5CS(=O)(=O)C5)nc4-c4ccc(F)cc4)ncnc3o2)n(C)n1] | 3-{4-[6-(1,3-dimethyl-1H- pyrazol-5-yl)furo[2,3- d]pyrimidin-4-yl]-3-(4- fluorophenyl)-1H-pyrazol-1-yl}- 1λ6-thietane-1,1-dione |
| 244 | 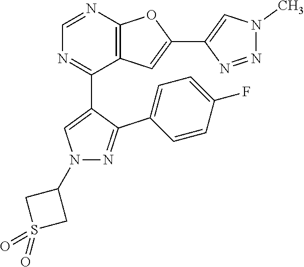 [SMILES: Cn1cc(-c2cc3c(-c4cn(C5CS(=O)(=O)C5)nc4-c4ccc(F)cc4)ncnc3o2)nn1] | 3-{3-(4-fluorophenyl)-4-[6-(1- methyl-1H-1,2,3-triazol-4- yl)furo[2,3-d]pyrimidin-4-yl]- 1H-pyrazol-1-yl}-126-thietane- 1,1-dione |
| 245 | 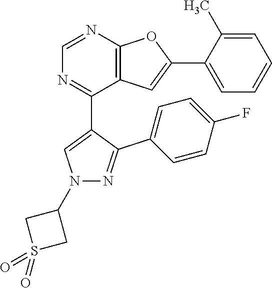 [SMILES: Cc1ccccc1-c1cc2c(-c3cn(C4CS(=O)(=O)C4)nc3-c3ccc(F)cc3)ncnc2o1] | 3-{3-(4-fluorophenyl)-4-[6-(2- methylphenyl)furo[2,3- d]pyrimidin-4-yl]-1H-pyrazol-1- yl}-1λ6-thietane-1,1-dione |
| 246 | 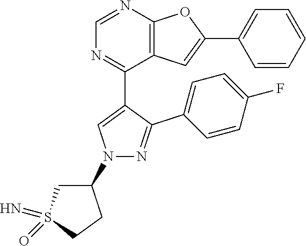 [SMILES: N=[S@]1(=O)CC[C@H](n2cc(-c3ncnc4oc(-c5ccccc5)cc34)c(-c3ccc(F)cc3)n2)C1] | (1S,3S)-3-[3-(4-fluorophenyl)-4- (6-phenylfuro[2,3-d]pyrimidin-4- yl)-1H-pyrazol-1-yl]-1-imino- 1λ6-thiolan-1-one (absolute stereochemistry arbitrarily assigned) |
| 247 | 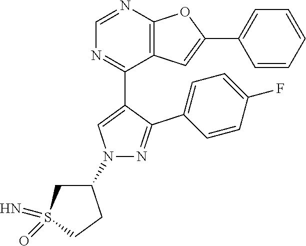 [SMILES: N=[S@@]1(=O)CC[C@@H](n2cc(-c3ncnc4oc(-c5ccccc5)cc34)c(-c3ccc(F)cc3)n2)C1] | (1R,3R)-3-[3-(4-fluorophenyl)-4- (6-phenylfuro[2,3-d]pyrimidin-4- yl)-1H-pyrazol-1-yl]-1-imino- 1λ6-thiolan-1-one (absolute stereochemistry arbitrarily assigned) |
| 248 | 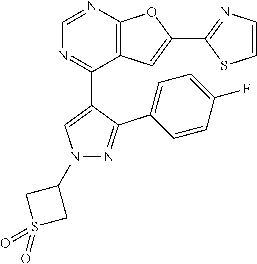 [SMILES: O=S1(=O)CC(n2cc(-c3ncnc4oc(-c5nccs5)cc34)c(-c3ccc(F)cc3)n2)C1] | 3-{3-(4-fluorophenyl)-4-[6-(1,3- thiazol-2-yl)furo[2,3- d]pyrimidin-4-yl]-1H-pyrazol-1- yl}-1λ6-thietane-1,1-dione |
| 249 | 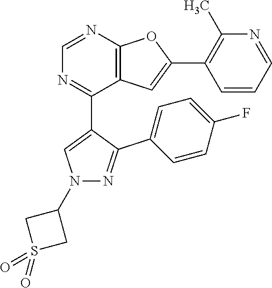 [SMILES: Cc1ncccc1-c1cc2c(-c3cn(C4CS(=O)(=O)C4)nc3-c3ccc(F)cc3)ncnc2o1] | 3-{3-(4-fluorophenyl)-4-[6-(2- methylpyridin-3-yl)furo[2,3- d]pyrimidin-4-yl]-1H-pyrazol-1- yl}-1λ6-thietane-1,1-dione |
| 250 | 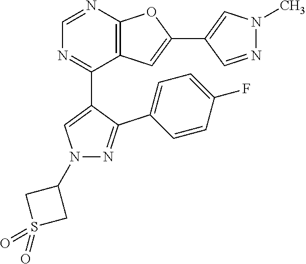 [SMILES: Cn1cc(-c2cc3c(-c4cn(C5CS(=O)(=O)C5)nc4-c4ccc(F)cc4)ncnc3o2)cn1] | 3-{3-(4-fluorophenyl)-4-[6-(1- methyl-1H-pyrazol-4- yl)furo[2,3-d]pyrimidin-4-yl]- 1H-pyrazol-1-yl}-1λ6-thietane- 1,1-dione |
| 251 | 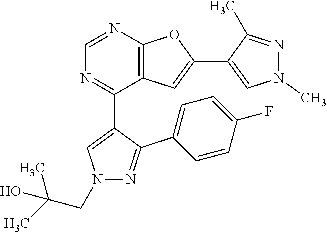 [SMILES: Cc1nn(C)cc1-c1cc2c(-c3cn(CC(C)(C)O)nc3-c3ccc(F)cc3)ncnc2o1] | 1-{4-[6-(1,3-dimethyl-1H- pyrazol-4-yl)furo[2,3- d]pyrimidin-4-yl]-3-(4- fluorophenyl)-1H-pyrazol-1-yl}- 2-methylpropan-2-ol |
| 252 | 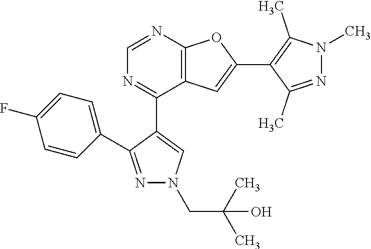 [SMILES: Cc1nn(C)c(C)c1-c1cc2c(-c3cn(CC(C)(C)O)nc3-c3ccc(F)cc3)ncnc2o1] | 1-{3-(4-fluorophenyl)-4-[6- (1,3,5-trimethyl-1H-pyrazol-4- yl)furo[2,3-d]pyrimidin-4-yl]- 1H-pyrazol-1-yl}-2- methylpropan-2-ol |
| 253 | 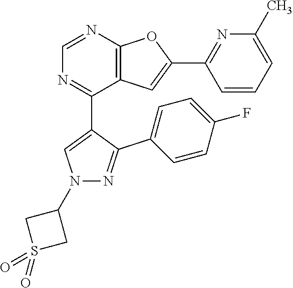 [SMILES: Cc1cccc(-c2cc3c(-c4cn(C5CS(=O)(=O)C5)nc4-c4ccc(F)cc4)ncnc3o2)n1] | 3-{3-(4-fluorophenyl)-4-[6-(6- methylpyridin-2-yl)furo[2,3- d]pyrimidin-4-yl]-1H-pyrazol-1- yl}-1λ6-thietane-1,1-dione |
| 254 | 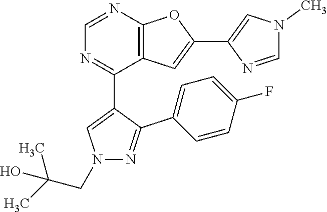 [SMILES: Cn1cnc(-c2cc3c(-c4cn(CC(C)(C)O)nc4-c4ccc(F)cc4)ncnc3o2)c1] | 1-{3-(4-fluorophenyl)-4-[6-(1- methyl-1H-imidazol-4- yl)furo[2,3-d]pyrimidin-4-yl]- 1H-pyrazol-1-yl}-2- methylpropan-2-ol |
| 255 | 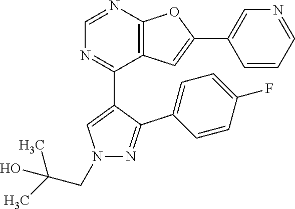 [SMILES: CC(C)(O)Cn1cc(-c2ncnc3oc(-c4cccnc4)cc23)c(-c2ccc(F)cc2)n1] | 1-{3-(4-fluorophenyl)-4-[6- (pyridin-3-yl)furo[2,3- d]pyrimidin-4-yl]-1H-pyrazol-1- yl}-2-methylpropan-2-ol |
| 256 | 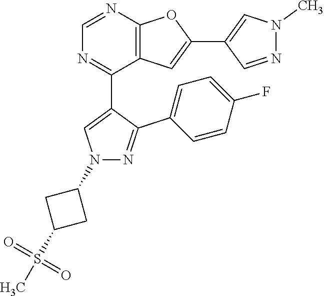 [SMILES: Cn1cc(-c2cc3c(-c4cn([C@H]5C[C@@H](S(C)(=O)=O)C5)nc4-c4ccc(F)cc4)ncnc3o2)cn1] | 4-{3-(4-fluorophenyl)-1-[(1s,3s)- 3-(methanesulfonyl)cyclobutyl]- 1H-pyrazol-4-yl}-6-(1-methyl- 1H-pyrazol-4-yl)furo[2,3- d]pyrimidine |
| 257 | 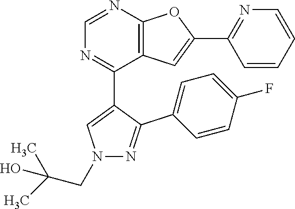 [SMILES: CC(C)(O)Cn1cc(-c2ncnc3oc(-c4ccccn4)cc23)c(-c2ccc(F)cc2)n1] | 1-{3-(4-fluorophenyl)-4-[6- (pyridin-2-yl)furo[2,3- d]pyrimidin-4-yl]-1H-pyrazol-1- yl}-2-methylpropan-2-ol |
| 258 | 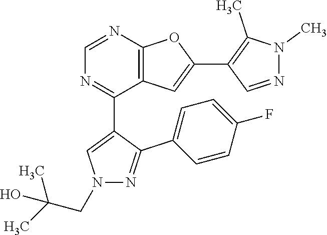 [SMILES: Cc1c(-c2cc3c(-c4cn(CC(C)(C)O)nc4-c4ccc(F)cc4)ncnc3o2)cnn1C] | 1-{4-[6-(1,5-dimethyl-1H- pyrazol-4-yl)furo[2,3- d]pyrimidin-4-yl]-3-(4- fluorophenyl)-1H-pyrazol-1-yl}- 2-methylpropan-2-ol |
| 259 | 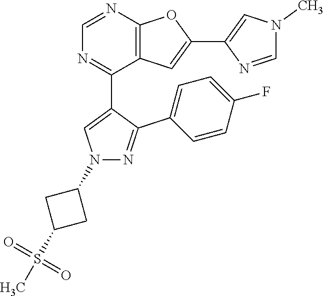 [SMILES: Cn1cnc(-c2cc3c(-c4cn([C@H]5C[C@@H](S(C)(=O)=O)C5)nc4-c4ccc(F)cc4)ncnc3o2)c1] | 4-{3-(4-fluorophenyl)-1-[(1s,3s)- 3-(methanesulfonyl)cyclobutyl]- 1H-pyrazol-4-yl}-6-(1-methyl- 1H-imidazol-4-yl)furo[2,3- d]pyrimidine |
| 260 | 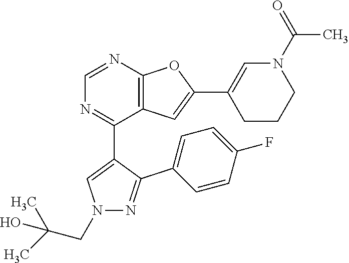 [SMILES: CC(=O)N1C=C(c2cc3c(-c4cn(CC(C)(C)O)nc4-c4ccc(F)cc4)ncnc3o2)CCC1] | 1-[5-{4-[3-(4-fluorophenyl)-1- (2-hydroxy-2-methylpropyl)-1H- pyrazol-4-yl]furo[2,3- d]pyrimidin-6-yl}-3,4- dihydropyridin-1(2H)-yl]ethan- 1-one |
| 261 | 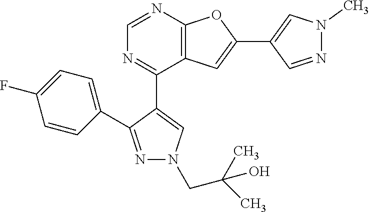 [SMILES: Cn1cc(-c2cc3c(-c4cn(CC(C)(C)O)nc4-c4ccc(F)cc4)ncnc3o2)cn1] | 1-{3-(4-fluorophenyl)-4-[6-(1- methyl-1H-pyrazol-4- yl)furo[2,3-d]pyrimidin-4-yl]- 1H-pyrazol-1-yl}-2- methylpropan-2-ol |
| 262 | 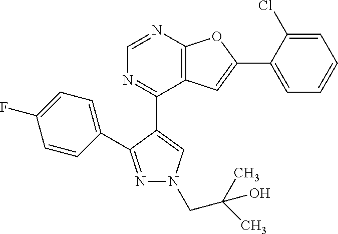 [SMILES: CC(C)(O)Cn1cc(-c2ncnc3oc(-c4ccccc4Cl)cc23)c(-c2ccc(F)cc2)n1] | 1-{4-[6-(2- chlorophenyl)furo[2,3- d]pyrimidin-4-yl]-3-(4- fluorophenyl)-1H-pyrazol-1-yl}- 2-methylpropan-2-ol |
| 263 | 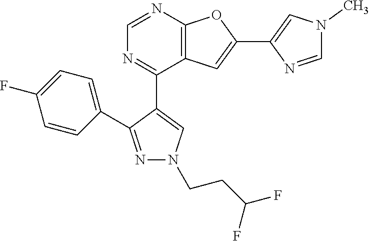 [SMILES: Cn1cnc(-c2cc3c(-c4cn(CCC(F)F)nc4-c4ccc(F)cc4)ncnc3o2)c1] | 4-[1-(3,3-difluoropropyl)-3-(4- fluorophenyl)-1H-pyrazol-4-yl]- 6-(1-methyl-1H-imidazol-4- yl)furo[2,3-d]pyrimidine |
| 264 | 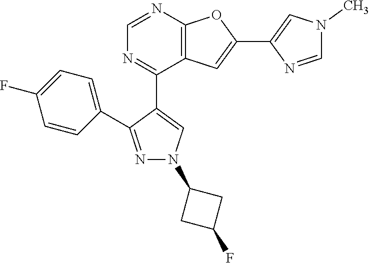 [SMILES: Cn1cnc(-c2cc3c(-c4cn([C@H]5C[C@@H](F)C5)nc4-c4ccc(F)cc4)ncnc3o2)c1] | 4-{1-[(1s,3s)-3- fluorocyclobutyl]-3-(4- fluorophenyl)-1H-pyrazol-4-yl}- 6-(1-methyl-1H-imidazol-4- yl)furo[2,3-d]pyrimidine |
| 265 | 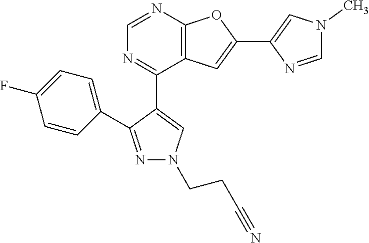 [SMILES: Cn1cnc(-c2cc3c(-c4cn(CCC#N)nc4-c4ccc(F)cc4)ncnc3o2)c1] | 3-{3-(4-fluorophenyl)-4-[6-(1- methyl-1H-imidazol-4- yl)furo[2,3-d]pyrimidin-4-yl]- 1H-pyrazol-1-yl}propanenitrile |
| 266 | 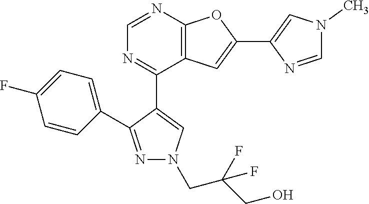 [SMILES: Cn1cnc(-c2cc3c(-c4cn(CC(F)(F)CO)nc4-c4ccc(F)cc4)ncnc3o2)c1] | 2,2-difluoro-3-{3-(4- fluorophenyl)-4-[6-(1-methyl- 1H-imidazol-4-yl)furo[2,3- d]pyrimidin-4-yl]-1H-pyrazol-1- yl}propan-1-ol |
| 267 | 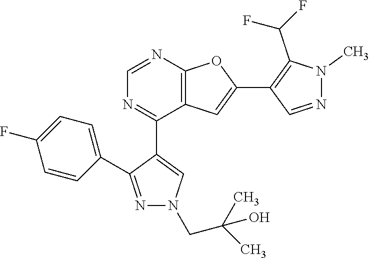 [SMILES: Cn1ncc(-c2cc3c(-c4cn(CC(C)(C)O)nc4-c4ccc(F)cc4)ncnc3o2)c1C(F)F] | 1-[4-{6-[5-(difluoromethyl)-1- methyl-1H-pyrazol-4- yl]furo[2,3-d]pyrimidin-4-yl}-3- (4-fluorophenyl)-1H-pyrazol-1- yl]-2-methylpropan-2-ol |
| 268 | 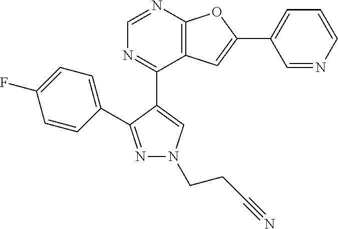 [SMILES: N#CCCn1cc(-c2ncnc3oc(-c4cccnc4)cc23)c(-c2ccc(F)cc2)n1] | 3-{3-(4-fluorophenyl)-4-[6- (pyridin-3-y1)furo[2,3- d]pyrimidin-4-yl]-1H-pyrazol-1- yl}propanenitrile |
| 269 | 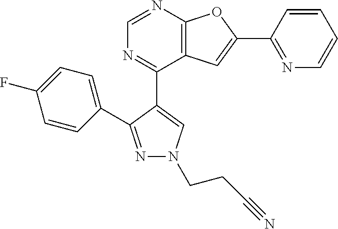 [SMILES: N#CCCn1cc(-c2ncnc3oc(-c4ccccn4)cc23)c(-c2ccc(F)cc2)n1] | 3-{3-(4-fluorophenyl)-4-[6- (pyridin-2-yl)furo[2,3- d]pyrimidin-4-yl]-1H-pyrazol-1- yl}propanenitrile |
| 270 | 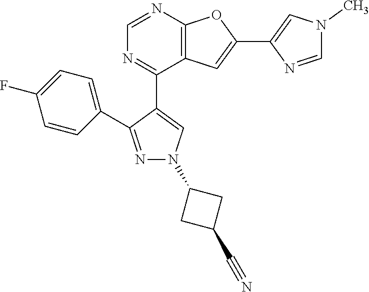 [SMILES: Cn1cnc(-c2cc3c(-c4cn([C@H]5C[C@H](C#N)C5)nc4-c4ccc(F)cc4)ncnc3o2)c1] | (1r,3r)-3-{3-(4-fluorophenyl)-4- [6-(1-methyl-1H-imidazol-4- yl)furo[2,3-d]pyrimidin-4-yl]- 1H-pyrazol-1-yl}cyclobutane-1- carbonitrile |
| 271 | 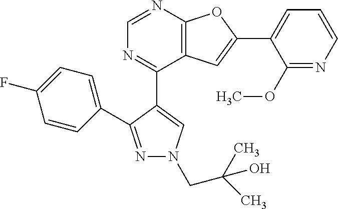 [SMILES: COc1ncccc1-c1cc2c(-c3cn(CC(C)(C)O)nc3-c3ccc(F)cc3)ncnc2o1] | 1-{3-(4-fluorophenyl)-4-[6-(2- methoxypyridin-3-yl)furo[2,3- d]pyrimidin-4-yl]-1H-pyrazol-1- yl}-2-methylpropan-2-ol |
| 272 | 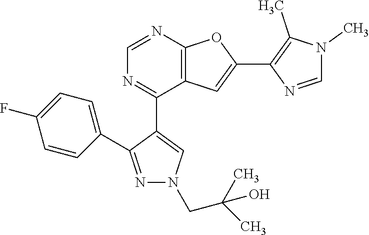 [SMILES: Cc1c(-c2cc3c(-c4cn(CC(C)(C)O)nc4-c4ccc(F)cc4)ncnc3o2)ncn1C] | 1-{4-[6-(1,5-dimethyl-1H- imidazol-4-yl)furo[2,3- d]pyrimidin-4-yl]-3-(4- fluorophenyl)-1H-pyrazol-1-yl}- 2-methylpropan-2-ol |
| 273 | 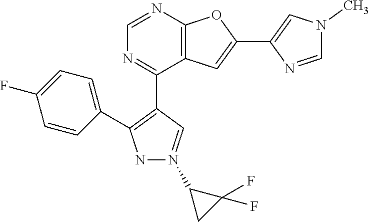 | 4-{1-[(1R)-2,2- difluorocyclopropyl]-3-(4- fluorophenyl)-1H-pyrazol-4-yl}- 6-(1-methyl-1H-imidazol-4- yl)furo[2,3-d]pyrimidine (absolute stereochemistry arbitrarily assigned) |
| 274 | 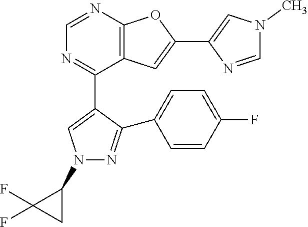 [SMILES: Cn1cnc(-c2cc3c(-c4cn([C@H]5CC5(F)F)nc4-c4ccc(F)cc4)ncnc3o2)c1] | 4-{1-[(1S)-2,2- difluorocyclopropyl]-3-(4- fluorophenyl)-1H-pyrazol-4-yl}- 6-(1-methyl-1H-imidazol-4- yl)furo[2,3-d]pyrimidine (absolute stereochemistry arbitrarily assigned) |
| 275 | 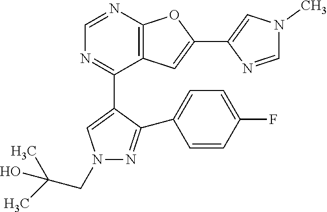 [SMILES: Cn1cnc(-c2cc3c(-c4cn(CC(C)(C)O)nc4-c4ccc(F)cc4)ncnc3o2)c1] | 1-{3-(4-fluorophenyl)-4-[6-(1- methyl-1H-pyrazol-3- yl)furo[2,3-d]pyrimidin-4-yl]- 1H-pyrazol-1-yl}-2- methylpropan-2-ol |
| 276 | 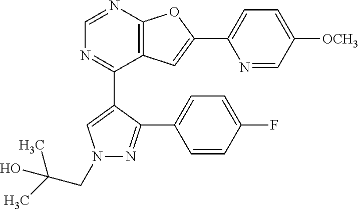 [SMILES: COc1ccc(-c2cc3c(-c4cn(CC(C)(C)O)nc4-c4ccc(F)cc4)ncnc3o2)nc1] | 1-{3-(4-fluorophenyl)-4-[6-(5- methoxypyridin-2-yl)furo[2,3- d]pyrimidin-4-yl]-1H-pyrazol-1- yl}-2-methylpropan-2-ol |
| 277 | 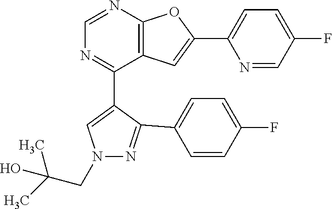 [SMILES: CC(C)(O)Cn1cc(-c2ncnc3oc(-c4ccc(F)cn4)cc23)c(-c2ccc(F)cc2)n1] | 1-{3-(4-fluorophenyl)-4-[6-(5- fluoropyridin-2-yl)furo[2,3- d]pyrimidin-4-yl]-1H-pyrazol-1- yl}-2-methylpropan-2-ol |
| 278 | 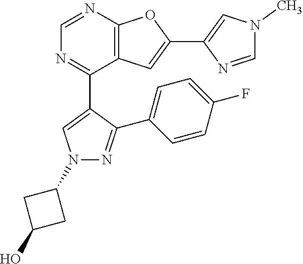 [SMILES: Cn1cnc(-c2cc3c(-c4cn([C@H]5C[C@H](O)C5)nc4-c4ccc(F)cc4)ncnc3o2)c1] | (1r,3r)-3-{3-(4-fluorophenyl)-4- [6-(1-methyl-1H-imidazol-4- yl)furo[2,3-d]pyrimidin-4-yl]- 1H-pyrazol-1-yl}cyclobutan-1-ol |
| 279 | 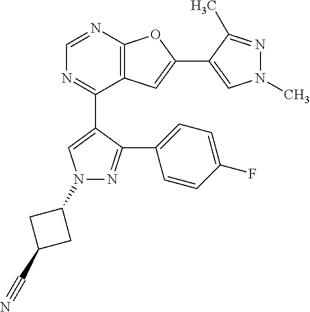 [SMILES: Cc1nn(C)cc1-c1cc2c(-c3cn([C@H]4C[C@H](C#N)C4)nc3-c3ccc(F)cc3)ncnc2o1] | (1r,3r)-3-{4-[6-(1,3-dimethyl- 1H-pyrazol-4-yl)furo[2,3- d]pyrimidin-4-yl]-3-(4- fluorophenyl)-1H-pyrazol-1- yl}cyclobutane-1-carbonitrile |
| 280 | 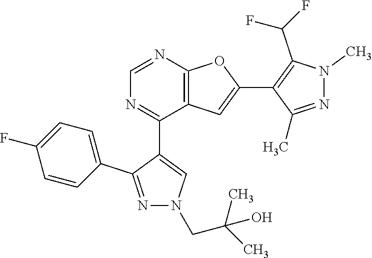 [SMILES: Cc1nn(C)c(C(F)F)c1-c1cc2c(-c3cn(CC(C)(C)O)nc3-c3ccc(F)cc3)ncnc2o1] | 1-[4-{6-[5-(difluoromethyl)-1,3- dimethyl-1H-pyrazol-4- yl]furo[2,3-d]pyrimidin-4-yl}-3- (4-fluorophenyl)-1H-pyrazol-1- yl]-2-methylpropan-2-ol |
| 281 | 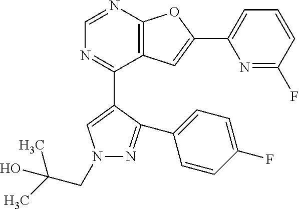 [SMILES: CC(C)(O)Cn1cc(-c2ncnc3oc(-c4cccc(F)n4)cc23)c(-c2ccc(F)cc2)n1] | 1-{3-(4-fluorophenyl)-4-[6-(6- fluoropyridin-2-yl)furo[2,3- d]pyrimidin-4-yl]-1H-pyrazol-1- yl}-2-methylpropan-2-ol |
| 282 | 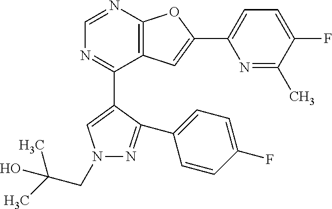 [SMILES: Cc1nc(-c2cc3c(-c4cn(CC(C)(C)O)nc4-c4ccc(F)cc4)ncnc3o2)ccc1F] | 1-{4-[6-(5-fluoro-6- methylpyridin-2-yl)furo[2,3- d]pyrimidin-4-yl]-3-(4- fluorophenyl)-1H-pyrazol-1-yl}- 2-methylpropan-2-ol |
| 283 | 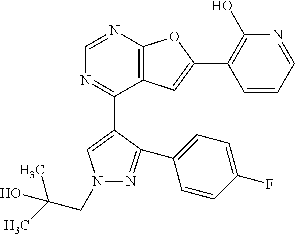 [SMILES: CC(C)(O)Cn1cc(-c2ncnc3oc(-c4cccnc4O)cc23)c(-c2ccc(F)cc2)n1] | 3-{4-[3-(4-fluorophenyl)-1-(2- hydroxy-2-methylpropyl)-1H- pyrazol-4-yl]furo[2,3- d]pyrimidin-6-yl}pyridin-2-ol |
| 284 | 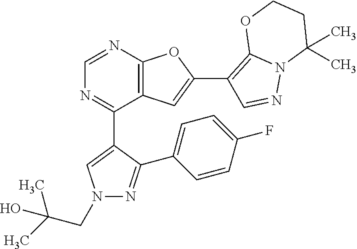 [SMILES: CC(C)(O)Cn1cc(-c2ncnc3oc(-c4cnn5c4OCCC5(C)C)cc23)c(-c2ccc(F)cc2)n1] | 1-(4-(6-(7,7-dimethyl-6,7- dihydro-5H-pyrazolo[5,1- b][1,3]oxazin-3-yl)furo[2,3- d]pyrimidin-4-yl)-3-(4- fluorophenyl)-1H-pyrazol-1-yl)- 2-methylpropan-2-ol |
| 285 | 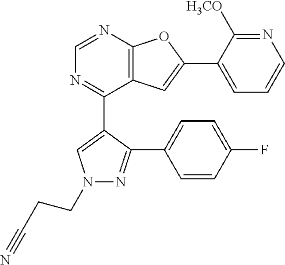 [SMILES: COc1ncccc1-c1cc2c(-c3cn(CCC#N)nc3-c3ccc(F)cc3)ncnc2o1] | 3-{3-(4-fluorophenyl)-4-[6-(2- methoxypyridin-3-yl)furo[2,3- d]pyrimidin-4-yl]-1H-pyrazol-1- yl}propanenitrile |
| 286 | 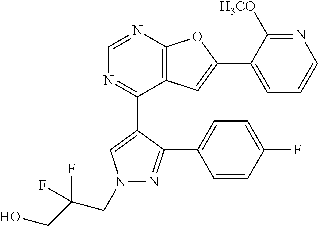 [SMILES: COc1ncccc1-c1cc2c(-c3cn(CC(F)(F)CO)nc3-c3ccc(F)cc3)ncnc2o1] | 2,2-difluoro-3-{3-(4- fluorophenyl)-4-[6-(2- methoxypyridin-3-yl)furo[2,3- d]pyrimidin-4-yl]-1H-pyrazol-1- yl}propan-1-ol |
| 287 | 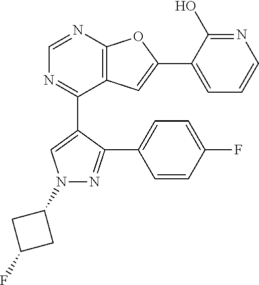 [SMILES: Oc1ncccc1-c1cc2c(-c3cn([C@H]4C[C@@H](F)C4)nc3-c3ccc(F)cc3)ncnc2o1] | 3-(4-{1-[(1s,3s)-3- fluorocyclobutyl]-3-(4- fluorophenyl)-1H-pyrazol-4- yl}furo[2,3-d]pyrimidin-6- yl)pyridin-2-ol |

Further provided herein are pharmaceutical compositions comprising an amount of a compound of (XII), or a pharmaceutically acceptable salt, solvate, or stereoisomer thereof, and one or more pharmaceutically acceptable excipients.

### Methods of Treatment

Further provided herein are methods of treating cancer in a subject in need thereof, comprising administering to the subject a therapeutically effective amount of compound of Formula (I), (II), (IIa), (IIb), (IIc), (IId), (IIe), (III), (IV), (V), (VI), (VII), (VIII), (VIIIa), (IX), (X), (XI), or (XII) or a pharmaceutically acceptable salt, or a pharmaceutical composition disclosed herein comprising a compound of Formula (I), pharmaceutically acceptable salt thereof. Also provided herein are methods of treating cancer in a subject, wherein the cancer has been determined to comprise a C797S mutation in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. Further provided herein are methods of treating cancer in a subject, wherein the cancer has been determined to comprise a C797S mutation in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In further embodiments are provided methods of treating metastatic cancer in a subject, wherein the metastatic cancer has been determined to comprise a C797S mutation in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments are provided such methods, wherein the cancer has been further determined to comprise an exon 19 deletion mutation in the epidermal growth factor receptor (EGFR) protein. In some embodiments are provided such methods, wherein the cancer has been further determined to comprise an L858R mutation in the epidermal growth factor receptor (EGFR) protein. In some embodiments are provided such methods, wherein the cancer has been further determined not to comprise a T790M mutation in the epidermal growth factor receptor (EGFR) protein. In some embodiments are provided such methods, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some embodiments, the cancer is selected from the group consisting of breast cancer, colon cancer, lung cancer, non-small cell lung cancer, gastric cancer, prostate cancer, and colorectal cancer. In other embodiments, the cancer comprises one or more central nervous system (CNS) metastases (e.g., brain metastases).

Also provided herein are methods of treating cancer in a subject, wherein the cancer has been determined to comprise L858R and C797S mutations in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments are provided such methods, wherein the cancer has been further determined not to comprise a T790M mutation in the epidermal growth factor receptor (EGFR) protein. In some embodiments are provided such methods, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some embodiments, the cancer is selected from the group consisting of breast cancer, colon cancer, lung cancer, non-small cell lung cancer, gastric cancer, prostate cancer, and colorectal cancer. In other embodiments, the cancer comprises one or more central nervous system (CNS) metastases (e.g., brain metastases).

Further provided herein are methods of treating metastatic cancer in a subject, wherein the metastatic cancer has been determined to comprise exon 19 deletion and C797S mutations in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments the cancer has been further determined not to comprise a T790M mutation in the epidermal growth factor receptor (EGFR) protein. In some embodiments are provided such methods, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some embodiments, the cancer is selected from the group consisting of breast cancer, colon cancer, lung cancer, non-small cell lung cancer, gastric cancer, prostate cancer, and colorectal cancer. In other embodiments, the cancer comprises one or more central nervous system (CNS) metastases (e.g., brain metastases).

Also provided herein are methods of treating a tumor in a subject, wherein the tumor has been determined to comprise has been determined to comprise a C797S mutation in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. Further provided herein are methods of treating a tumor in a subject following resection of the tumor, wherein the tumor has been determined to comprise a C797S mutation in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. Also provided herein are methods of treating a metastatic tumor in a subject, wherein the metastatic tumor has been determined to comprise a C797S mutation in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments the cancer has been further determined not to comprise a T790M mutation in the epidermal growth factor receptor (EGFR) protein. In some embodiments are provided such methods, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some embodiments, the cancer is selected from the group consisting of breast cancer, colon cancer, lung cancer, non-small cell lung cancer, gastric cancer, prostate cancer, and colorectal cancer. In other embodiments, the cancer comprises one or more central nervous system (CNS) metastases (e.g., brain metastases).

Also provided herein are methods of treating cancer in a subject, wherein the cancer has progressed following administration to the subject of one or more prior therapy that exhibits inhibitory activity against cancer comprising a T790M mutation in the epidermal growth factor receptor (EGFR) protein, and wherein the cancer been determined to comprise a C797S mutation in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

Further provided herein are methods of treating cancer in a subject, wherein the cancer has progressed following administration to the subject of osimertinib, and wherein the cancer been determined to comprise a C797S mutation in the epidermal growth factor receptor (EGFR) protein, comprising administering to the subject a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments are provided such methods, wherein the cancer is neuroblastoma, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, or pituitary adenoma. In some embodiments, the cancer is selected from the group consisting of breast cancer, colon cancer, lung cancer, non-small cell lung cancer, gastric cancer, prostate cancer, and colorectal cancer. In other embodiments, the cancer comprises one or more central nervous system (CNS) metastases (e.g., brain metastases).

In some embodiments are provided any of the methods of treatment disclosed herein, wherein the cancer is a locally advanced cancer. In other embodiments are provided such methods, wherein the cancer is an unresectable cancer. In other embodiments are provided such methods, wherein the cancer is a resectable cancer. In other embodiments are provided such methods, wherein the cancer is a metastatic cancer

In further embodiments are provided methods of treatment described herein, wherein the subject has received one or more prior therapy for treatment of the cancer prior to administration to the subject of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments, the subject has received one or more prior therapy for treatment of the cancer prior to administration to the subject of a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, wherein the one more prior therapy exhibits inhibitory activity against cancer comprising a T790M mutation in the epidermal growth factor receptor (EGFR) protein. In some embodiments, the one more prior therapy exhibits inhibitory activity against cancer comprising a T790M mutation in the epidermal growth factor receptor (EGFR) protein is osimertinib. In some embodiments of the methods disclosed herein, the subject has been administered osimertinib prior to administration to the subject of the compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

In some embodiments are provided any of the methods of treatment disclosed herein, wherein the compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, is administered to the subject in need thereof orally, parentally, intravenously, subcutaneously, or intracerebrally.

### Combinations

In some embodiments are provided any of the methods of treatment disclosed herein, wherein the method further comprises administering to the subject in need thereof one or more additional anticancer agents. In some embodiments, the one or more additional anticancer agents comprises one or more agents selected from HER2 inhibitors, HER2-CD3 bispecific antibodies, HER2-immune targeting bispecific antibodies, anti-HER2 chimeric antigen receptor (CAR) T cells, anti-HER2 chimeric antigen receptor (CAR) cytotoxic T-lymphocytes (CTLs), anti-HER2 chimeric antigen receptor (CAR) natural killer (NK) cells, anti-HER2 chimeric antigen receptor (CAR) cytokine-induced killer (CIK) cells, epidermal growth factor receptor (EGFR) inhibitors, poly-ADP-ribose polymerase (PARP) inhibitors, PD-1 inhibitors, PD-L1 inhibitors, Phosphoinositide 3-kinase (PI3K) inhibitors, and chemotherapeutic agents. In some embodiments are provided any such methods of treatment described herein, wherein the one or more additional cancer agents are selected from the group consisting of erlotinib, gefitinib, afatanib, trastuzumab, trastuzumab and hyaluronidase, capecitabine, trastuzumab and capecitabine, tucatinib, lapatinib, neratinib, dacomitinib, pertuzumab, margetuximab, trastuzumab emtansine, trastuzumab deruxtecan, ZW49 (Zymeworks), A166 (Klaus Pharma), ARX788 (Ambrx), RC48-ADC (RemeGen), vic-trastuzumab duocarmazine, zanidatamab (ZW25), zenocutuzamab (MCLA-128), ISB 1302, poziotinib, pyrotinib, mobocertinib (TAK-788), and BDTX-189, osimertinib, cetuximab, panitumumab, necitumumab, vandetanib, brigatinib, icotinib, niraparib, olaparib, talazoparib, rucaparib, veliparib, iniparib, pamiparib (BGB-290), CEP-9722, E7016, pembrolizumab, nivolumab, cemiplimab, JTX-4014, spartalizumab (PDR001), camrelizumab (SHR1210), sintilimab (1B1308), tislelizumab (BGB-A317), toripalimab (JS 001), dostarlimab (TSR-042, WBP-285), INCMGA00012 (MGA012), AMP-224, AMP-514, atezolizumab, avelumab, durvalumab, KN035, CK-301, AUNP12, CA-170, and BMS-986189. In some embodiments, the one or more additional cancer agents is selected from erlotinib, gefitinib, and afatanib. In some embodiments, the one or more additional cancer agents is erlotinib. In some embodiments, the one or more additional cancer agents is gefitinib. In some embodiments, the one or more additional cancer agents is afatanib.

In other embodiments are any such methods of treatment disclosed herein, wherein the method further comprises treating the subject in need thereof with radiation.

In some embodiments are any of the methods of treatment disclosed herein wherein the subject is a human.

Further provided herein are uses of a composition comprising a of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, for manufacture of a medicament for treatment of a subject having cancer, wherein the cancer comprises a C797S mutation in the epidermal growth factor receptor (EGFR) protein.

Further provided herein are uses of a composition comprising a of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, for manufacture of a medicament for the treatment of cancer in a subject, wherein the cancer comprises (a) one or more central nervous system (CNS) metastases, and (b) a C797S mutation in the epidermal growth factor receptor (EGFR) protein.

Also provided herein are kits comprising (i) a composition comprising a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, and (ii) instructions for using the kit in treating cancer in a human, wherein the cancer comprises a C797S mutation in the epidermal growth factor receptor (EGFR) protein. In some embodiment, the kit further comprises one or more additional anticancer agents. In some embodiments, the one or more additional anticancer agents comprises one or more agents selected from HER2 inhibitors, HER2-CD3 bispecific antibodies, HER2-immune targeting bispecific antibodies, anti-HER2 chimeric antigen receptor (CAR) T cells, anti-HER2 chimeric antigen receptor (CAR) cytotoxic T-lymphocytes (CTLs), anti-HER2 chimeric antigen receptor (CAR) natural killer (NK) cells, anti-HER2 chimeric antigen receptor (CAR) cytokine-induced killer (CIK) cells, epidermal growth factor receptor (EGFR) inhibitors, poly-ADP-ribose polymerase (PARP) inhibitors, PD-1 inhibitors, PD-L1 inhibitors, Phosphoinositide 3-kinase (PI3K) inhibitors, and chemotherapeutic agents. In further embodiment, the one or more additional cancer agents are selected from the group consisting of erlotinib, gefitinib, afatanib, trastuzumab, trastuzumab and hyaluronidase, capecitabine, trastuzumab and capecitabine, tucatinib, lapatinib, neratinib, dacomitinib, pertuzumab, margetuximab, trastuzumab emtansine, trastuzumab deruxtecan, ZW49 (Zymeworks), A166 (Klaus Pharma), ARX788 (Ambrx), RC48-ADC (RemeGen), vic-trastuzumab duocarmazine, zanidatamab (ZW25), zenocutuzamab (MCLA-128), ISB 1302, poziotinib, pyrotinib, mobocertinib (TAK-788), and BDTX-189, osimertinib, cetuximab, panitumumab, necitumumab, vandetanib, brigatinib, icotinib, niraparib, olaparib, talazoparib, rucaparib, veliparib, iniparib, pamiparib (BGB-290), CEP-9722, E7016, pembrolizumab, nivolumab, cemiplimab, JTX-4014, spartalizumab (PDR001), camrelizumab (SHR1210), sintilimab (1B1308), tislelizumab (BGB-A317), toripalimab (JS 001), dostarlimab (TSR-042, WBP-285), INCMGA00012 (MGA012), AMP-224, AMP-514, atezolizumab, avelumab, durvalumab, KN035, CK-301, AUNP12, CA-170, and BMS-986189. In some embodiments, the one or more additional cancer agents is selected from erlotinib, gefitinib, and afatanib. In some embodiments, the one or more additional cancer agents is erlotinib. In some embodiments, the one or more additional cancer agents is gefitinib. In some embodiments, the one or more additional cancer agents is afatanib.

Methods of Treatment in Conjunction with Biomarkers

Disclosed herein, in some embodiments, are methods of detecting the presence, absence, or level, of a biomarker. Such biomarkers may comprise genetic alterations in the gene encoding for certain proteins such as EGFR. The presence, absence, or level, of such biomarkers may be measured in a biological sample obtained from a subject, such as a sample of a solid tumor, such as a prostate cancer, or from a sample of a relevant biological fluid, such as a blood sample. In some instances, the methods of detection disclosed herein are useful for predicting a therapeutic response to a therapy described herein (e.g., an EGFR inhibitor) in, monitor the treatment using the therapy of, and treating with the therapy, a proliferative disease or condition described herein in a subject. In some embodiments, the presence, or an absence, and/or a level of expression of the one or more biomarkers is detected in the sample obtained from a subject by analyzing the genetic material in the sample. In some embodiments, the genetic material is obtained from blood, serum, plasma, sweat, hair, tears, urine, and other techniques known by one of skill in the art. In some embodiments the sample comprises circulating tumor RNA (ctRNA). In some embodiments the sample comprises peripheral blood mononuclear cells (PBMCs). In some cases, the genetic material is obtained from a tumor biopsy or liquid biopsy. In some embodiments, a tumor biopsy comprises a formalin-fixed paraffin embedded biopsy, a fresh frozen biopsy, a fresh biopsy, or a frozen biopsy. In some embodiments, a liquid biopsy comprises PBMCs, circulating tumor RNA, plasma cell-free RNA, or circulating tumor cells (CTCs). Tumor biopsies can undergo additional analytic processing for sample dissociation, cell sorting, and enrichment of cell populations of interest.

In some embodiments, methods of detecting a presence, absence, or level of a biomarker in the sample obtained from the subject involve detecting a nucleic acid sequence. In some cases, the nucleic acid sequence comprises deoxyribonucleic acid (DNA), such as in the case of detecting complementary DNA (cDNA) of an mRNA transcript. In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises ribonucleic acid (RNA). In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Disclosed herein, in some embodiments, the biomarker is detected by subjecting a sample obtained from the subject to a nucleic acid-based detection assay. In some instances, the nucleic acid-based detection assay comprises quantitative polymerase chain reaction (qPCR), gel electrophoresis (including for e.g., Northern or Southern blot), immunochemistry, in situ hybridization such as fluorescent in situ hybridization (FISH), cytochemistry, microarray, or sequencing. In some embodiments, the sequencing technique comprises next generation sequencing. In some embodiments, the methods involve a hybridization assay such as fluorogenic qPCR (e.g., TaqMan™, SYBR green, SYBR green I, SYBR green II, SYBR gold, ethidium bromide, methylene blue, Pyronin Y, DAPI, acridine orange, Blue View or phycoerythrin), which involves a nucleic acid amplification reaction with a specific primer pair, and hybridization of the amplified nucleic acid probes comprising a detectable moiety or molecule that is specific to a target nucleic acid sequence. In some instances, a number of amplification cycles for detecting a target nucleic acid in a qPCR assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles. For TaqMan™ methods, the probe may be a hydrolysable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles. In some instances, hybridization may occur at standard hybridization temperatures, e.g., between about 35° C. and about 65° C. in a standard PCR buffer.

An additional exemplary nucleic acid-based detection assay comprises the use of nucleic acid probes conjugated or otherwise immobilized on a bead, multi-well plate, or other substrate, wherein the nucleic acid probes are configured to hybridize with a target nucleic acid sequence. In some instances, the nucleic acid probe is specific to one or more gene products described herein. In some instances, the nucleic acid probe specific to a biomarker comprises a nucleic acid probe sequence sufficiently complementary to the polynucleotide sequence of the biomarker. In some instances, the biomarker comprises a transcribed polynucleotide sequence (e.g., RNA, cDNA). In some embodiments, the nucleic acid probe can be, for example, a full-length cDNA, or a portion thereof, such as an oligonucleotide of at least about 7, 8, 9, 10, 11, 12, 13, 14, 15, 20, 25, 30, 35, 40, 45, or 50 nucleotides in length and sufficient to specifically hybridize under standard hybridization conditions to the target nucleic acid sequence. In some embodiments, the target nucleic acid sequence is immobilized on a solid surface and contacted with a probe, for example by running the isolated target nucleic acid sequence on an agarose gel and transferring the target nucleic acid sequence from the gel to a membrane, such as nitrocellulose. In some embodiments, the probe(s) are immobilized on a solid surface, for example, in an Affymetrix gene chip array, and the probe(s) are contacted with the target nucleic acid sequence.

In some embodiments, the term “probe” with regards to nucleic acids, refers to any nucleic acid molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate, and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3,4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some embodiments, detecting the one or more biomarkers, such as gene products in a predictive response signature (PRS), comprises sequencing genetic material obtained from a sample from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modem sequencing technologies such as Illumina sequencing (e.g., Solexa), Roche 454 sequencing, Ion torrent sequencing, and SOLiD sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

Disclosed herein are methods comprising: (a) providing a sample obtained from a subject with a proliferative disease or condition (e.g., cancer); (b) assaying to detect in the sample obtained from the subject a presence or absence of the relevant biomarker; and (c) detecting the presence or absence of the biomarker in the sample using the methods described herein. In some cases, a hybridization assay, such as those described herein, is used to detect the biomarker in the sample. Exemplary probe sequences that are hybridizable to a target nucleic acid sequence (e.g., one or more genes in the biomarker, such as the PRS) comprise at least 10, but no more than 100 contiguous nucleotides comprising the relevant sequence. In some cases, RNA sequencing (RNAseq) is used to detect the one or more biomarkers.

Detection of the relevant biomarker, in some cases, involves amplification of the subject's nucleic acid by the polymerase chain reaction (PCR). In some embodiments, the PCR assay involves use of a pair of primers capable of amplifying at least about 10 contiguous nucleobases within a nucleic acid sequence, thereby amplifying the one or more gene products in the biomarker. In fluorogenic quantitative PCR, quantitation is based on amount of fluorescence signals (TaqMan and SYBR green). In some embodiments, the nucleic acid probe is conjugated to a detectable molecule. The detectable molecule may be a fluorophore. The nucleic acid probe may also be conjugated to a quencher.

In some embodiments, the assay for detecting the presence or absence of a relevant biomarker comprises reverse-transcribing the relevant mRNA molecule to produce a corresponding complementary DNA (cDNA) molecule). In some embodiments, the assay further comprises contacting the cDNA molecule with a nucleic acid probe comprising a nucleic acid sequence that is complementary to a nucleic acid sequence of the cDNA molecule. In some embodiments, the assay comprises detecting a double-stranded hybridization product between the nucleic acid probe and the cDNA molecule. In some embodiments, the hybridization product is further amplified using a pair of primers. In some embodiments, the primers comprises a first primer with a nucleic acid sequence comprising at least 10 but not more than 50 contiguous nucleic acids within a relevant nucleic acid sequence that binds to a top strand of the double-stranded hybridization product; and a second primer with a nucleic acid sequence comprising at least 10 but not more than 50 contiguous nucleic acids within a nucleic acid sequence that is reverse complement to the relevant nucleic acid sequence that binds to a bottom strand of the double-stranded hybridization product.

Disclosed herein, in some embodiments, are methods comprising preparing a complementary DNA (cDNA) library. In some embodiments, the cDNA library is sequenced using suitable sequence methodologies disclosed herein. In some embodiments, the cDNA library is labeled, a plurality of nucleic acid probes is generated, and fixed to an immobile surface (such as a microarray). In some embodiments, the plurality of nucleic acid probes is capable of hybridizing to at least about 10 contiguous nucleotides of the two or more genes in a sample obtained from the subject. In some embodiments, detecting the presence of or absence of a biomarker includes detecting a high or a low level of expression of the two or more genes as compared to a reference level.

Disclosed herein, in some embodiments, genetic material is extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

Circulating Tumor DNA (ctDNA) and RNA (ctRNA)

In some aspects, circulating tumor DNA (ctDNA) is used to assess the presence of certain DNA molecules and circulating tumor RNA (ctRNA) is used to assess the expression levels of RNA molecules, shed by the tumor into the blood stream.

In some embodiments, detection of ctDNA or ctRNA is useful, for example, for detecting and diagnosing a tumor. Because tumor DNA and RNA has acquired multiple genetic mutations, leading to tumor development, ctDNA and ctRNA are not an exact match to the subject's DNA and RNA, respectively. Finding DNA and RNA with genetic differences aids in tumor detection. Diagnosing the type of tumor using ctDNA or ctRNA can reduce the need for getting a sample of the tumor tissue (tumor biopsy), which can be challenging when a tumor is difficult to access, such as a tumor in the brain or lung.

In some embodiments, a decrease in the quantity of ctDNA or ctRNA suggests the solid tumor is shrinking and treatment with a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof is effective. In some embodiments, a lack of ctDNA or ctRNA in the bloodstream indicates that the cancer has not returned after treatment with a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

Described herein are methods of assessing genetic alterations by ctDNA or ctRNA genomic profiling. In some embodiments, the genomic profiling is performed after each treatment cycle with a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments, the gene mutations indicate that the cancer is becoming resistant to the treatment with a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof. In some embodiments, the lack of gene mutations indicate that the cancer is not becoming resistant to the treatment with a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof.

The compounds of Formula (I), (II), (IIa), (IIb), (IIc), (IId), (IIe), (III), (IV), (V), (VI), (VII), (VIII), (VIIIa), (IX), (X), (XI), or (XII) may be administered as prodrugs. Thus certain derivatives of the compounds, which may have little or no pharmacological activity themselves can, when administered to a mammal, be converted into a compound having the desired activity, for example, by hydrolytic cleavage. Such derivatives are referred to as “prodrugs.” Prodrugs can, for example, be produced by replacing appropriate functionalities present in the compound of Formula (I), (II), (IIa), (IIb), (IIe), (IId), (IIe), (III), (IV), (V), (VI), (VII), (VIII), (VIIIa), (IX), (X), (XI), or (XII) with certain moieties known to those skilled in the art. See, e.g. “Pro-drugs as Novel Delivery Systems”, Vol. 14, ACS Symposium Series (T Higuchi and W Stella) and “Bioreversible Carriers in Drug Design”, Pergamon Press, 1987 (ed. E B Roche, American Pharmaceutical Association), the disclosures of which are incorporated herein by reference in their entireties. Some examples of such prodrugs include: an ester moiety in the place of a carboxylic acid functional group; an ether moiety or an amide moiety in place of an alcohol functional group; and an amide moiety in place of a primary or secondary amino functional group. Examples of replacement groups are known to those of skill in the art. See, e.g. “Design of Prodrugs” by H Bundgaard (Elsevier, 1985), the disclosure of which is incorporated herein by reference in its entirety.

Salts of the compounds disclosed herein can be prepared according to methods known to those of skill in the art. Examples of salts include, but are not limited to, acetate, acrylate, benzenesulfonate, benzoate (such as chlorobenzoate, methylbenzoate, dinitrobenzoate, hydroxybenzoate, and methoxybenzoate), bicarbonate, bisulfate, bisulfite, bitartrate, borate, bromide, butyne-1,4-dioate, calcium edetate, camsylate, carbonate, chloride, caproate, caprylate, clavulanate, citrate, decanoate, dihydrochloride, dihydrogenphosphate, edetate, edisylate, estolate, esylate, ethylsuccinate, formate, fumarate, gluceptate, gluconate, glutamate, glycollate, glycollylarsanilate, heptanoate, hexyne-1,6-dioate, hexylresorcinate, hydrabamine, hydrobromide, hydrochloride, gamma-hydroxybutyrate, iodide, isobutyrate, isothionate, lactate, lactobionate, laurate, malate, maleate, malonate, mandelate, mesylate, metaphosphate, methane-sulfonate, methylsulfate, monohydrogenphosphate, mucate, napsylate, naphthalene-1-sulfonate, naphthalene-2-sulfonate, nitrate, oleate, oxalate, pamoate (embonate), palmitate, pantothenate, phenylacetates, phenylbutyrate, phenylpropionate, phthalate, phosphate/diphosphate, polygalacturonate, propanesulfonate, propionate, propiolate, pyrophosphate, pyrosulfate, salicylate, stearate, subacetate, suberate, succinate, sulfate, sulfonate, sulfite, tannate, tartrate, teoclate, tosylate, triethiodode, and valerate salts.

The compounds of Formula (I), (II), (IIa), (IIb), (IIc), (IId), (IIe), (III), (IV), (V), (VI), (VII), (VIII), (VIIIa), (IX), (X), (XI), or (XII) that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention can be prepared by treating the base compound with a substantially equivalent amount of the selected mineral or organic acid in an aqueous solvent medium or in a suitable organic solvent, such as methanol or ethanol. Upon evaporation of the solvent, the desired solid salt is obtained. The desired acid salt can also be precipitated from a solution of the free base in an organic solvent by adding an appropriate mineral or organic acid to the solution.

The compounds of Formula (I), (II), (IIa), (IIb), (IIc), (IId), (IIe), (III), (IV), (V), (VI), (VII), (VIII), (VIIIa), (IX), (X), (XI), or (XII) that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium calcium and magnesium, etc. These salts can be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

If the compound of Formula (I), (II), (IIa), (IIb), (IIc), (IId), (IIe), (III), (IV), (V), (VI), (VII), (VIII), (VIIIa), (IX), (X), (XI), or (XII) is a base, the desired salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the compound of Formula (I), (II), (IIa), (IIb), (IIc), (IId), (IIe), (III), (IV), (V), (VI), (VII), (VIII), (VIIIa), (IX), (X), (XI), or (XII) is an acid, the desired salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum, and lithium.

If the compound of Formula (I), (II), (IIa), (IIb), (IIc), (IId), (IIe), (III), (IV), (V), (VI), (VII), (VIII), (VIIIa), (IX), (X), (XI), or (XII) is a solid, it is understood by those skilled in the art that the compounds or salts thereof may exist in different crystal or polymorphic forms, all of which are intended to be within the scope of the present invention and specified formulas.

Also provided herein are isotopically-labeled compounds of Formula (I), (II), (IIa), (IIb), (IIc), (IId), (IIe), (III), (IV), (V), (VI), (VII), (VIII), (VIIIa), (IX), (X), (XI), or (XII), wherein one or more atoms is replaced by an atom having the same atomic number, but an atomic mass or mass number different from the atomic mass or mass number usually found in nature. Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ¹⁵S. Certain isotopically-labeled compounds of the invention, for example, those incorporating a radioactive isotope, are useful in drug and/or substrate tissue distribution studies. The radioactive isotopes tritium, ³H, and carbon-14, ¹⁴C, are particularly useful for this purpose in view of their ease of incorporation and ready means of detection. Substitution with heavier isotopes such as deuterium, ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of Formula (I), (II), (IIa), (IIb), (IIc), (IId), (IIe), (III), (IV), (V), (VI), (VII), (VIII), (VIIIa), (IX), (X), (XI), or (XII) can generally be prepared by conventional techniques known to those skilled in the art or by processes analogous to those described herein, using an appropriate isotopically-labeled reagent in place of the non-labeled reagent otherwise employed.

In one aspect, the compositions of compounds of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof, described herein are used for the treatment of cancer in a subject. In one embodiment, such compositions are in the form of suitable dosage forms. Suitable dosage forms include, for example, liquids, suspensions, powders for reconstitution, tablets, pills, sachets, or capsules of hard or soft gelatin (See, e.g., Remington: The Science and Practice of Pharmacy (Gennaro, 21st Ed. Mack Pub. Co., Easton, PA (2005)).

The compounds of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof, may be formulated into pharmaceutical compositions as described below in any pharmaceutical form recognizable to the skilled artisan as being suitable. Pharmaceutical compositions of the invention comprise a therapeutically effective amount of at least one compound of the present invention and an inert, pharmaceutically acceptable carrier or diluent.

The pharmaceutical carriers employed may be either solid or liquid. Exemplary solid carriers are lactose, sucrose, talc, gelatin, agar, pectin, acacia, magnesium stearate, stearic acid, and the like. Exemplary liquid carriers are syrup, peanut oil, olive oil, water, and the like. Similarly, the inventive compositions may include time-delay or time-release material known in the art, such as glyceryl monostearate or glyceryl distearate alone or with a wax, ethylcellulose, hydroxypropylmethylcellulose, methylmethacrylate or the like. Further additives or excipients may be added to achieve the desired formulation properties. For example, a bioavailability enhancer, such as Labrasol, Gelucire or the like, or formulator, such as CMC (carboxy-methylcellulose), PG (propyleneglycol), or PEG (polyethyleneglycol), may be added. Gelucire., a semi-solid vehicle that protects active ingredients from light, moisture, and oxidation, may be added, e.g., when preparing a capsule formulation.

If a solid carrier is used, the preparation can be tableted, placed in a hard gelatin capsule in powder or pellet form, or formed into a troche or lozenge. The amount of solid carrier may vary, but generally will be from about 25 mg to about 1 g. If a liquid carrier is used, the preparation may be in the form of syrup, emulsion, soft gelatin capsule, sterile injectable solution or suspension in an ampoule or vial or non-aqueous liquid suspension. If a semi-solid carrier is used, the preparation may be in the form of hard and soft gelatin capsule formulations. The inventive compositions are prepared in unit-dosage form appropriate for the mode of administration, e.g. parenteral or oral administration.

To obtain a stable water-soluble dose form, a salt of a compound of the present invention may be dissolved in an aqueous solution of an organic or inorganic acid, such as a 0.3 M solution of succinic acid or citric acid. If a soluble salt form is not available, the agent may be dissolved in a suitable co-solvent or combinations of co-solvents. Examples of suitable co-solvents include alcohol, propylene glycol, polyethylene glycol 300, polysorbate 80, glycerin and the like in concentrations ranging from 0 to 60% of the total volume. In an exemplary embodiment, a compound of the present invention is dissolved in DMSO and diluted with water. The composition may also be in the form of a solution of a salt form of the active ingredient in an appropriate aqueous vehicle such as water or isotonic saline or dextrose solution.

Proper formulation is dependent upon the route of administration selected. For injection, the agents of the compounds of the present invention may be formulated into aqueous solutions, preferably in physiologically compatible buffers such as Hanks solution, Ringer's solution, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers known in the art. Such carriers enable the compounds of the invention to be formulated as tablets, pills, dragees, capsules, liquids, gels, syrups, slurries, suspensions, and the like, for oral ingestion by a subject to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions.

Alternatively, the active ingredient may be in powder form for constitution with a suitable vehicle, e.g. sterile pyrogen-free water, before use.

In addition to the formulations described above, the compounds disclosed herein may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt. A pharmaceutical carrier for hydrophobic compounds is a co-solvent system comprising benzyl alcohol, a non-polar surfactant, a water-miscible organic polymer, and an aqueous phase. The co-solvent system may be a VPD co-solvent system. VPD is a solution of 3% w/v benzyl alcohol, 8% w/v of the non-polar surfactant polysorbate 80, and 65% w/v polyethylene glycol 300, made up to volume in absolute ethanol. The VPD co-solvent system (VPD: 5W) contains VPD diluted 1:1 with a 5% dextrose in water solution. This co-solvent system dissolves hydrophobic compounds well, and itself produces low toxicity upon systemic administration. The proportions of a co-solvent system may be suitably varied without destroying its solubility and toxicity characteristics. Furthermore, the identity of the co-solvent components may be varied: for example, other low-toxicity non-polar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide (DMSO) also may be employed, although usually at the cost of greater toxicity due to the toxic nature of DMSO. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. These carriers and excipients may provide marked improvement in the bioavailability of poorly soluble drugs. Examples of such carriers or excipients include calcium carbonate, calcium phosphate, sugars, starches, cellulose derivatives, gelatin, and polymers such as polyethylene glycols.

Further, the pharmaceutical composition may be incorporated into a skin patch for delivery of the drug directly onto the skin.

It will be appreciated that the actual dosages of the agents of this invention will vary according to the particular agent being used, the particular composition formulated, the mode of administration, and the particular site, host, and disease being treated. Those skilled in the art using conventional dosage-determination tests in view of the experimental data for a given compound may ascertain optimal dosages for a given set of conditions. For oral administration, an exemplary daily dose generally employed will be from about 0.001 to about 1000 mg/kg of body weight, with courses of treatment repeated at appropriate intervals.

Furthermore, the pharmaceutically acceptable formulations disclosed herein may contain a compound as disclosed herein, or a salt or solvate thereof, in an amount of about 10 mg to about 2000 mg, or from about 10 mg to about 1500 mg, or from about 10 mg to about 1000 mg, or from about 10 mg to about 750 mg, or from about 10 mg to about 500 mg, or from about 25 mg to about 500 mg, or from about 50 to about 500 mg, or from about 100 mg to about 500 mg.

Additionally, the pharmaceutically acceptable formulations disclosed herein may contain a compound as disclosed herein, or a pharmaceutically acceptable salt thereof, in an amount from about 0.5 w/w % to about 95 w/w %, or from about 1 w/w % to about 95 w/w %, or from about 1 w/w % to about 75 w/w %, or from about 5 w/w % to about 75 w/w %, or from about 10 w/w % to about 75 w/w %, or from about 10 w/w % to about 50 w/w %.

The compounds of the present invention, or salts or solvates thereof, may be administered to a mammal suffering from abnormal cell growth, such as a human, either alone or as part of a pharmaceutically acceptable formulation, once a day, twice a day, three times a day, or four times a day, or even more frequently.

Those of ordinary skill in the art will understand that with respect to the compounds Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof, the particular pharmaceutical formulation, the dosage, and the number of doses given per day to a mammal requiring such treatment, are all choices within the knowledge of one of ordinary skill in the art and can be determined without undue experimentation.

Dosages of compositions described herein can be determined by any suitable method. Maximum tolerated doses (MTD) and maximum response doses (MRD) for the compounds of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof, can be determined via established animal and human experimental protocols as well as in the examples described herein. For example, toxicity and therapeutic efficacy of the compounds of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof, can be determined by standard pharmaceutical procedures in cell cultures or experimental animals, including, but not limited to, for determining the LD50 (the dose lethal to 50% of the population) and the ED50 (the dose therapeutically effective in 50% of the population). The dose ratio between the toxic and therapeutic effects is the therapeutic index and it can be expressed as the ratio between LD50 and ED50. The data obtained from cell culture assays and animal studies can be used in formulating a range of dosage for use in human. The dosage of such compounds lies preferably within a range of circulating concentrations that include the ED50 with minimal toxicity. The dosage may vary within this range depending upon the dosage form employed and the route of administration utilized. Additional relative dosages, represented as a percent of maximal response or of maximum tolerated dose, are readily obtained via the protocols.

In some embodiments, the amount of the compounds of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof, comprising a formulation that corresponds to such an amount varies depending upon factors such as the particular salt or form, disease condition and its severity, the identity (e.g., age, weight, sex) of the subject or host in need of treatment, but can nevertheless be determined according to the particular circumstances surrounding the case, including, e.g., the specific agent being administered, the liquid formulation type, the condition being treated, and the subject or host being treated.

In some embodiments, a compound of Formulae (I), Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, is administered in an amount between about 10 mg to 500 mg per day. In some embodiments, a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, is administered in an amount between about 100 mg to about 400 mg per day. In some embodiments, a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, is administered in an amount between about 150 mg to about 350 mg per day. In some embodiments, a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, is administered to the subject in need thereof in an amount between about 100 mg to about 1000 mg, or from about 100 mg to about 900 mg, or from about 100 mg to about 800 mg, or from about 100 mg to about 700 mg, or from about 100 mg to about 600 mg, or from about 100 mg to about 500 mg per day.

In some embodiments, a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt thereof, is administered to the subject in need thereof in an amount of about 100 mg, or about 150 mg, or about 200 mg, or about 250 mg, or about 300 mg, or about 350 mg, or about 400 mg, or about 450 mg, or about 500 mg, or about 550 mg, or about 600 mg, or about 650 mg, or about 700 mg, or about 750 mg, or about 800 mg, or about 850 mg, or about 900 mg, or about 1000 mg per day.

In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the subject. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the subject.

In certain embodiments wherein the subject's condition does not improve, upon the doctor's discretion the administration of a composition described herein are administered chronically, that is, for an extended period of time, including throughout the duration of the subject's life in order to ameliorate or otherwise control or limit the symptoms of the subject's disease. In other embodiments, administration of a composition continues until complete or partial response of a disease.

In some embodiments, a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof, is administered to a subject in need thereof once a day. In some embodiments, a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof, is administered to a subject in need thereof twice a day. In some embodiments, a compound of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof is administered to a subject in need thereof three times a day.

In some instances, the methods described herein comprise administering the compositions and formulations comprising the compounds of Formula (I), (II), (IIa), (IIb), (IIc), (IId), (IIe), (III), (IV), (V), (VI), (VII), (VIII), (VIIIa), (IX), (X), (XI), or (XII) in combination with one or more additional therapeutic agents, to the subject or subject in need thereof in multiple cycles repeated on a regular schedule with periods of rest in between each cycle. For example, in some instances, treatment given for one week followed by three weeks of rest is one treatment cycle.

The length of a treatment cycle depends on the treatment being given. In some embodiments, the length of a treatment cycle ranges from two to six weeks. In some embodiments, the length of a treatment cycle ranges from three to six weeks. In some embodiments, the length of a treatment cycle ranges from three to four weeks. In some embodiments, the length of a treatment cycle is three weeks (or 21 days). In some embodiments, the length of a treatment cycle is four weeks (28 days). In some embodiments, the length of a treatment cycle is 56 days. In some embodiments, a treatment cycle lasts one, two, three, or four weeks. In some embodiments, a treatment cycle lasts three weeks. In some embodiments, a treatment cycle lasts four weeks. The number of treatment doses scheduled within each cycle also varies depending on the drugs being given.

### Kits and Articles of Manufacture

Disclosed herein, in certain embodiments, are kits and articles of manufacture for use with one or more methods and compositions described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded, or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

In certain embodiments, the pharmaceutical compositions are presented in a pack or dispenser device which contains one or more unit dosage forms containing a compound provided herein. The pack, for example, contains metal or plastic foil, such as a blister pack. In one embodiment, the pack or dispenser device is accompanied by instructions for administration. In one embodiment, the pack or dispenser is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for drugs, or the approved product insert. In one embodiment, compositions containing a compound provided herein formulated in a compatible pharmaceutical carrier are also prepared, placed in an appropriate container, and labeled for treatment of an indicated condition.

### Methods of Preparation

Compounds of Formula (I), (II), (IIa), (IIb), (IIc), (IId), (IIe), (III), (IV), (V), (VI), (VII), (VIII), (VIIIa), (IX), (X), (XI), or (XII), or a pharmaceutically acceptable salt, solvate, or stereoisomer thereof, may be prepared using the reaction routes and synthetic schemes described below, employing the techniques available in the art using starting materials that are readily available. The preparation of certain embodiments of the present invention is described in detail in the following examples, but those of ordinary skill in the art will recognize that the preparations described may be readily adapted to prepare other embodiments of the present invention. For example, the synthesis of non-exemplified compounds according to the invention may be performed by modifications apparent to those skilled in the art, e.g. by appropriately protecting interfering groups, by changing to other suitable reagents known in the art, or by making routine modifications of reaction conditions. Alternatively, other reactions referred to herein or known in the art will be recognized as having adaptability for preparing other compounds of the invention.

The compounds of Formula (I), (II), (IIa), (IIb), (IIc), (IId), (IIe), (III), (IV), (V), (VI), (VII), (VIII), (VIIIa), (IX), (X), (XI), or (XII) may be prepared according to methods known to those of ordinary skill in the art without undue experimentation and in accordance with the methods described herein and using methods similar to those described below.

The compound of Example 4 can be prepared by allowing 7-bromo-2-phenyl-3H-imidazo[4,5-b]pyridine to react with a pinacolborane, such as the pyrazole shown below, in the presence of a palladium catalyst, a base such as sodium carbonate, and in solvent or mixture of solvents such as a mixture of dimethyl ether, ethanol, and water to afford the coupling product shown below. Those coupling products comprising a protecting group may be deprotected using methods known to those of ordinary skill in the art. For example, those coupling products comprising a triisopropylsilyl group may be deprotected using an acid, such as hydrochloric acid, in a solvent, such as methanol, to afford the deprotected product.

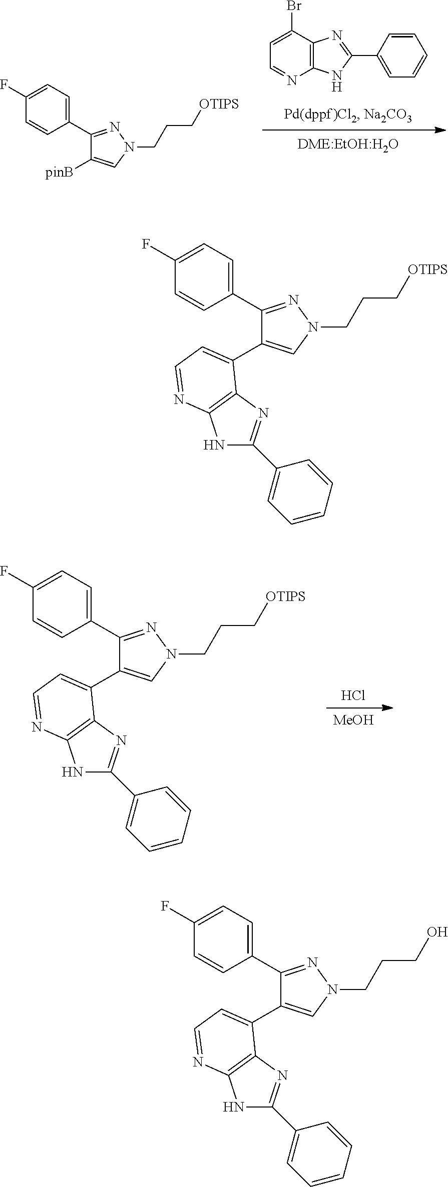

A compound comprising a pinacolborane may be prepared according to methods known to those of ordinary skill in the art and the methods described herein. For example, Intermediate F may be prepared from the corresponding halogen-containing intermediate by reaction with the n-butyl lithium and 2-isopropoxy-4,4,5,5-tetramethyl-1,3,2-dioxaborolane, in an aprotic solvent, such as tetrahydrofuran, and at a temperature in the range from −78° C. to 0° C., to afford the corresponding pinacolborane intermediate.

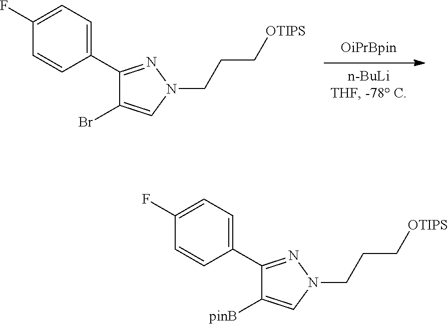

The corresponding halogen-containing intermediate, such as 4-bromo-3-(4-fluorophenyl)-1-(3-((triisopropylsilyl)oxy)propyl)-1H-pyrazole, may be prepared by reaction of an appropriate intermediate, such as 3-(4-fluorophenyl)-1H-pyrazole, with a halogenating reagent, such as n-bromosuccinimide, in an appropriate solvent, such as acetonitrile, and at a temperature in the range from 0° C. to 75° C., to afford the halogenated intermediate. The halogenated intermediate, may then be protected with an appropriate protecting group, such as a triisopropylsilyl group, by reaction with an appropriate reagent, such as triisopropylsilyl chloride, in a solvent, such as tetrahydrofuran, and in the presence of a base, such as cesium carbonate.

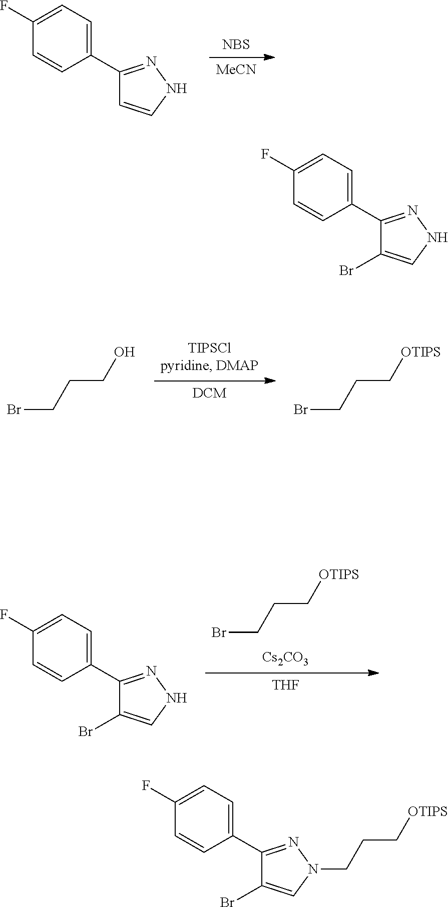

Compounds such as 7-bromo-2-phenyl-3H-imidazo[4,5-b]pyridine may be prepared according to methods known to those of ordinary skill in the art and by the methods disclosed herein. Compounds such as 7-bromo-2-phenyl-3H-imidazo[4,5-b]pyridine may be prepared from appropriate starting materials that are commercially available, or are readily prepared by methods known to those of ordinary skill in the art. For example, 7-bromo-2-phenyl-3H-imidazo[4,5-b]pyridine may be prepared by allowing 4-bromopyridine-2,3-diamine to react with benzoic acid and polyphosphoric acid at a temperature of 130° C. to afford 7-bromo-2-phenyl-3H-imidazo[4,5-b]pyridine. Compounds such as 4-bromopyridine-2,3-diamine are commercially available or may be prepared according to methods known to those of ordinary skill in the art.

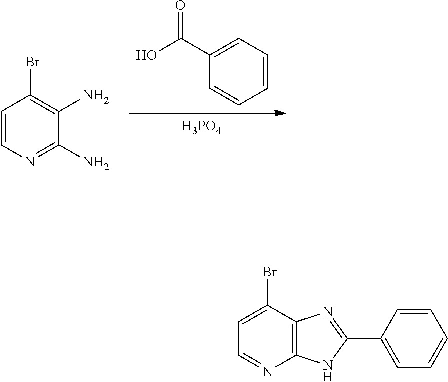

In the follow mg Preparations and Examples, “Ac” means acetyl, “ACN and “MeCN mean acetonitrile, “Me” means methyl, “Et” means ethyl, “Ph” means phenyl, “BOC”, “Boc” or “boc” means N-tert-butoxycarbonyl, “DCM” (CH₂Cl₂) means methylene chloride, “DIPEA” or “DIEA” means diisopropyl ethyl amine, “DMA” means N,N-dimethylacetamide, “DMF” means N—N-dimethyl formamide, “DMSO” means dimethylsulfoxide, “DPPP” means 1,3-bis(diphenylphosphino)propane, “DtBPF” means (di-tert-butylphosphino)ferrocene, “HOAc” means acetic acid, “IPA” means isopropyl alcohol, “min” means minute, “NMP” means 1-methyl 2-pyrrolidinone, “TEA” means triethyl amine, “TFA” means trifluoroacetic acid, “DCM” means dichloromethane, “EtOAc” and “EA” mean ethyl acetate, “MgSO₄” means magnesium sulphate, “Na₂SO₄” means sodium sulphate, “MeOH” means methanol, “Et₂O “means diethyl ether, “EtOH” means ethanol, “H₂O” means water, “HCl” means hydrochloric acid, “K₂CO₃” means potassium carbonate, “THE” means tetrahydrofuran, “DBU” means 1,8-diazabicyclo[5.4.0]undec-7-ene, “LiHMDS” or “LHMDS” means lithium hexamethyldisilazide, “TBME” or “MTBE” means tert-butyl methyl ether, “LDA” means lithium diisopropylamide, “N” means Normal, “M” means molar, “mL” means milliliter, “mmol” means millimoles, “μmol” means micromoles, “eq.” or “equiv.” means equivalent, “° C.” means degrees Celsius, “Pa” means pascals, “rt” or “RT” means room temperature, “h” means hours, “satd.” means saturated, “aq” means aqueous, “anhyd” means anhydrous, “quant.” means quantitative, “PE” means petroleum ether, “TBSCI” means tert-butyldimethylsilyl chloride, “TLC” means thin-layer chromatography, “HATU” means N-[(dimethylamino)-1H-1,2,3-triazolo-[4,5-b]pyridin-1-ylmethylene]-N-methylmethanaminium hexafluorophosphate N-oxide, “DME” means dimethyl ether, “DMAP” means 4-dimethylaminopyridine, “TsCl” means para-toluenesulfonyl chloride, “DIPEA” means N,N-diisopropylethylamine, “MsCl” means methanesulfonyl chloride, and “IPA” means iso-propyl alcohol.

### EXAMPLES

### Preparation of Intermediate Compounds

### Intermediate A. 4-Chloro-2-phenyl-1H-pyrrolo[2,3-b]pyridine

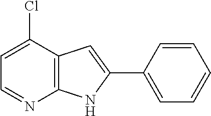 [SMILES: Clc1ccnc2[nH]c(-c3ccccc3)cc12]

### Step 1. 2-Phenyl-1H-pyrrolo[2,3-b]pyridine

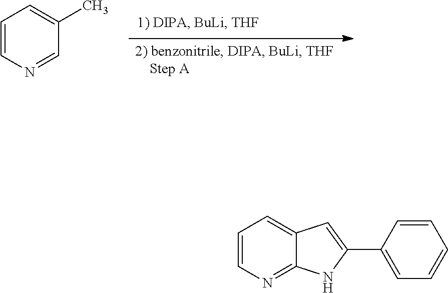

n-Butyllithium (9.45 mL, 23.6 mmol, 2.20 equiv, 2.5 M in hexane) was added dropwise under an argon atmosphere to a solution of diisopropylamine (DIPA) (2.39 g, 23.6 mmol, 2.20 equiv) in 16 mL dry THF at −78° C. The resulting solution was stirred at this temperature for 30 min. Half of the solution was transferred into a flame dried, argon-flooded flask and stored at 0° C. The temperature of the residual solution was raised to 0° C. and 3-methylpyridine (1.00 g, 10.7 mmol, 1.00 equiv) was added dropwise and the solution was stirred for further 30 min. Benzonitrile (1.11 g, 10.76 mmol, 1.00 equiv) was added dropwise and the temperature was kept below 10° C. The resulting mixture was stirred for further 90 min at <10° C. The stored lithium diisopropylamide (LDA)-solution was added and the resulting dark brown suspension was refluxed for 1 h. After cooling to RT, water (30 mL) was added and the organic layer was separated. The aqueous layer was extracted two times with EtOAc (2×50 mL). The combined organic layers were dried over Na₂SO₄ and the solvent was removed in vacuo. The residue was mixed with a small amount of MeOH and sonicated. The precipitate was filtered, washed with a small amount of cold MeOH and dried to afford the title compound (1 g, 48%) as a yellow solid. m/z (ESI, +ve ion)=195.20 [M+H]⁺.

### Step 2. 4-Chloro-2-phenyl-1H-pyrrolo[2,3-b]pyridine

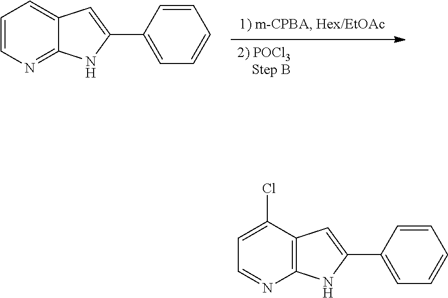

To a suspension of 2-phenyl-1H-pyrrolo[2,3-b]pyridine (1.00 g, 5.15 mmol, 1.00 equiv) in a 4:1 mixture of 175 mL hexane:EtOAc was added m-CPBA (1.60 g, 9.27 mmol, 1.80 equiv) portion wise. After the resulting mixture was stirred at RT overnight, the solvent was removed in vacuo and the residue was mixed with 100 mL of water and basified to a pH of 9 (saturated aq. K₂CO₃). The suspension was extracted with chloroform (4×50 mL) extensively, the organic layers were combined, dried over Na₂SO₄ and the solvent was removed in vacuo. The resulting N-oxide (600 mg) was added portion wise to 8 mL of POCl₃ at 0° C. After stirring for 15 min at 0° C., the mixture was heated at 100° C. overnight and then cooled to RT and poured into ice. The suspension was neutralized with a 0.1 M NaOH and extracted with EtOAc (3×30 mL). The combined organic layers were dried over Na₂SO₄ and the solvent was removed in vacuo. The residue was mixed with MeOH. The precipitate was filtered, washed with cold MeOH and dried to afford the title compound (422 mg, 36%) as a white solid. m/z (ESI, +ve ion)=229.10 [M+H]⁺.

### Intermediate B. 4-Chloro-1-(methoxymethyl)-2-phenylpyrrolo[2,3-b]pyridine

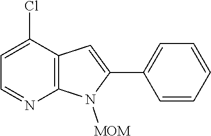

### Step 1. 4-Chloro-1-(methoxymethyl)-2-phenylpyrrolo[2,3-b]pyridine

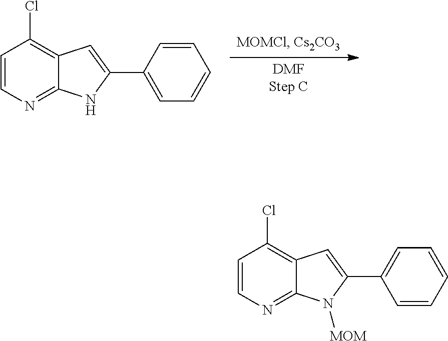

To a stirred mixture of 4-chloro-2-phenyl-1H-pyrrolo[2,3-b]pyridine (Intermediate A, 400 mg, 1.75 mmol, 1.00 equiv) in DMF (8.80 mL) were added Cs₂CO₃ (1.14 g, 3.50 mmol, 2.00 equiv) and MOM-Cl (140 mg, 2.10 mmol, 1.20 equiv) at 0° C. under nitrogen atmosphere. The resulting mixture was stirred for 30 min at RT under nitrogen atmosphere then quenched with saturated NH₄OAc (50 mL) at RT. The resulting mixture was extracted with EtOAc (3×100 mL). The combined organic layers were washed with brine (2×50 mL), dried over anhydrous Na₂SO₄. After filtration, the filtrate was concentrated under reduced pressure. The residue was purified by silica gel column chromatography, eluted with PE:EtOAc (2:1) to afford the title compound (261 mg, 55%) as a white solid. m/z (ESI, +ve ion)=273.10 [M+H]⁺.

### Intermediate C. 1-(Benzenesulfonyl)-4-bromopyrrolo[2,3-b]pyridine

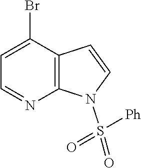 [SMILES: O=S(=O)(c1ccccc1)n1ccc2c(Br)ccnc21]

### Step 1. 1-(Benzenesulfonyl)-4-bromopyrrolo[2,3-b]pyridine

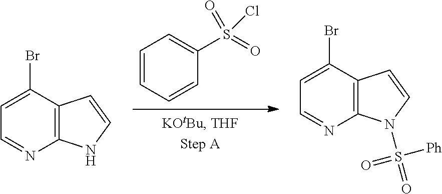

To a stirred mixture of 4-bromo-1H-pyrrolo[2,3-b]pyridine (42.9 g, 218 mmol, 1.00 equiv) in THF (400 mL) was added t-BuOK (26.87 g, 239.5 mmol, 1.1 equiv) at 0° C. under nitrogen atmosphere. The resulting mixture was stirred for 30 min at RT under nitrogen atmosphere. To the above mixture was added benzenesulfonyl chloride (42.30 g, 240 mmol, 1.1 equiv) at RT. After the resulting mixture was stirred for additional 16 h at RT, it was quenched by the addition of water (100 mL) at RT. The resulting mixture was extracted with EtOAc (3×100 mL). The combined organic layers were washed with brine (2×40 mL), dried over anhydrous Na₂SO₄. After filtration, the filtrate was concentrated under reduced pressure. The residue was purified by trituration with PE:EtOAc (1:1, 300 mL) to afford the title compound as a yellow solid. m/z (ESI, +ve ion)=336.86, 338.86 [M+H]⁺.

### Intermediate D. 1-(Benzenesulfonyl)-4-bromo-2-iodopyrrolo[2,3-b]pyridine

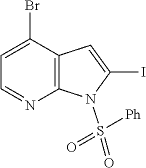 [SMILES: O=S(=O)(c1ccccc1)n1c(I)cc2c(Br)ccnc21]

### Step 1. 1-(Benzenesulfonyl)-4-bromo-2-iodopyrrolo[2,3-b]pyridine

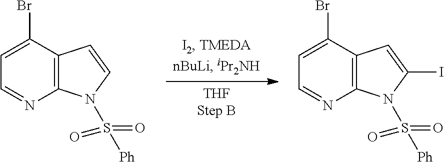 [SMILES: O=S(=O)(c1ccccc1)n1ccc2c(Br)ccnc21 ; "I2, TMEDAnBuLi, iPr2NH" ; "THFStep B" ; O=S(=O)(c1ccccc1)n1c(I)cc2c(Br)ccnc21]

To a stirred solution of diisopropylamine (2.61 g, 25.8 mmol, 1.3 equiv) in THF (80 mL) was added n-BuLi (9.54 mL, 23.8 mmol, 1.2 equiv, 2.5 M in hexane) dropwise at −78° C. under nitrogen atmosphere. The resulting mixture was stirred for 20 min at 0° C. under nitrogen atmosphere then was added dropwise into a solution of 1-(benzenesulfonyl)-4-bromopyrrolo[2,3-b]pyridine (Intermediate C, 6.7 g, 19.9 mmol, 1.00 equiv) and TMEDA (2.54 g, 21.9 mmol, 1.1 equiv) in THF (160 mL). The resulting mixture was stirred for 30 min at −78° C. under nitrogen atmosphere. To the above mixture was added I₂ (6.05 g, 23.8 mmol, 1.2 equiv) in THF (40 mL) at −78° C. The resulting mixture was stirred for additional 1 hour at −78° C. and then allowed to warm to RT. The reaction was quenched with sat. NH₄Cl (aq., 200 mL) and extracted with EtOAc (3×100 mL). The combined organic layers were washed with brine (2×60 mL), dried over anhydrous Na₂SO₄. After filtration, the filtrate was concentrated under reduced pressure. The residue was re-crystallized from hexane/ethyl acetate (5/1, 60 mL) to afford the title compound (6 g, 65%) as an off-white solid. m/z (ESI, +ve ion)=462.75, 464.75 [M+H]⁺.

### Intermediate E. 1-(Benzenesulfonyl)-4-bromo-2-phenylpyrrolo[2,3-b]pyridine

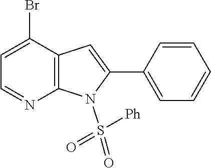 [SMILES: O=S(=O)(c1ccccc1)n1c(-c2ccccc2)cc2c(Br)ccnc21]

### Step 1. 1-(Benzenesulfonyl)-4-bromo-2-phenylpyrrolo[2,3-b]pyridine

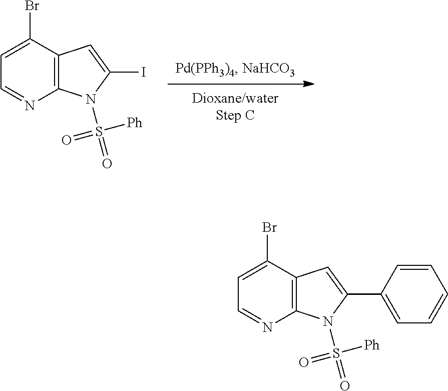

To a stirred mixture of 1-(benzenesulfonyl)-4-bromo-2-iodopyrrolo[2,3-b]pyridine (Intermediate D, 1.3 g, 2.81 mmol, 1.00 equiv) and phenylboronic acid (342 mg, 2.81 mmol, 1 equiv) in DMF (20 mL) and water (2 mL) were added Pd(PPh₃)₄ (162 mg, 0.140 mmol, 0.05 equiv) and NaHCO₃ (708 mg, 8.42 mmol, 3.00 equiv) at RT. The resulting mixture was stirred for 16 h at 100° C., and then concentrated under reduced pressure. The residue was purified by silica gel column chromatography, eluted with PE:EtOAc (7:1) to afford the title compound (960 mg, 83%) as a yellow solid. m/z (ESI, +ve ion)=412.90, 414.90 [M+H]⁺.

### Intermediate F. 3-(4-Fluorophenyl)-4-(4,4,5,5-tetramethyl-1,3,2-dioxaborolan-2-yl)-1-(3-((triisopropylsilyl)oxy)propyl)-1H-pyrazole

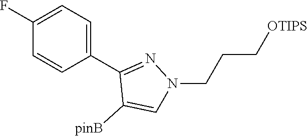

### Step 1. 4-Bromo-3-(4-fluorophenyl)-1H-pyrazole

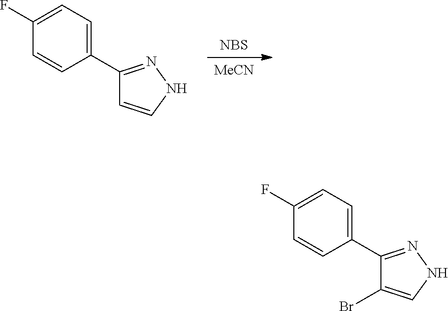 [SMILES: "MeCN" ; Fc1ccc(-c2cc[nH]n2)cc1 ; "NBS" ; Fc1ccc(-c2n[nH]cc2Br)cc1]

To a solution of 3-(4-Fluorophenyl)-1H-pyrazole (12.4 g, 76.7 mmol, 1.0 equiv) in MeCN (170 mL) that had been cooled to 0° C. using an ice-water bath was added n-Bromosuccinimide (12.9 g, 70.5 mmol, 0.92 equiv.) over 15 minutes. The reaction mixture was slowly warmed to RT over 20 minutes. The reaction mixture was diluted with a saturated solution of sodium thiosulfate (150 mL), water (250 mL) and EtOAc (150 mL) and mixed vigorously for 5 minutes. The layers were partitioned, and the aqueous layer was extracted with additional EtOAc (2×200 mL). The combined organic layers were washed with water (400 mL), dried under Na₂SO₄, filtered, and concentrated under reduced pressure to provide the title compound (17.2 g, 93%) as a solid. ¹H NMR (400 MHz, CDCl₃) δ 7.86-7.71 (m, 2H), 7.64 (s, 1H), 7.19-7.05 (m, 2H). m/z (ESI, +ve ion)=241.0, 243.0 [M+H]⁺.

### Step 2. (3-Bromopropoxy)triisopropylsilane

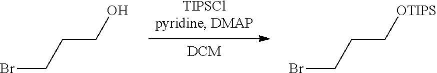

To a solution of 3-Bromo-1-propanol (6.51 mL, 69.8 mmol, 1.0 equiv.) in DCM (75 mL) was added imidazole (12.0 g, 174 mmol, 2.5 equiv.). The reaction mixture was stirred at RT for 30 min before triisopropylsilyl chloride (15.4 mL, 69.8 mmol, 1.0 equiv.) was added dropwise. The mixture was further stirred at 40° C. for 24 h. Upon cooling to RT H₂O (150 mL) was added and the resulting mixture was vigorously stirred for 1 h. The organic layer was washed with H₂O (2×50 mL), brine (50 mL), dried over Na₂SO₄, filtered, and concentrated under reduced pressure. The residue was eluted through a silica plug, using a 1:4 EtOAc:hexanes mixture as the eluent, to provide the title compound (14.8 g, 72%) as an oil. The material was used in the next steps without further purification. ¹H NMR (400 MHz, CDCl₃) δ 3.81 (t, J=5.7 Hz, 2H), 3.55 (t, J=6.5 Hz, 2H), 2.10-2.01 (m, 2H), 1.10-1.01 (m, 21H).

### Step 3. 4-Bromo-3-(4-fluorophenyl)-1-(3-((triisopropylsilyl)oxy)propyl)-1H-pyrazole

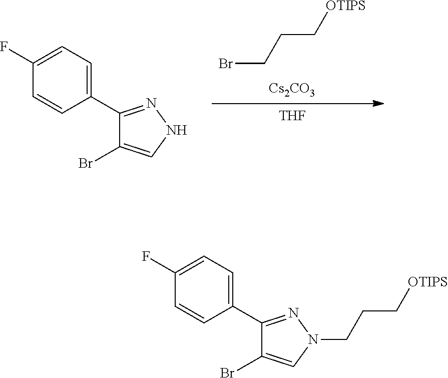

To a solution of 4-Bromo-3-(4-fluorophenyl)-1H-pyrazole (5.27 g, 21.9 mmol, 1.0 equiv.) in THF (109 mL), was added cesium carbonate (14.4 g, 43.7 mmol, 2.0 equiv.). After 5 minutes, (3-bromopropoxy)triisopropylsilane (9.68 g, 32.8 mmol, 1.5 equiv.) was added dropwise over 15 minutes. The reaction flask was equipped with a reflux condenser and warmed to 40° C. for 16 hours. The reaction mixture was concentrated under reduced pressure to half of its original volume. The resulting mixture was diluted with water (400 mL) and EtOAc (200 mL). The reaction mixture was stirred vigorously for 15 minutes, and the layers were separated. The aqueous layer was extracted with additional EtOAc (2×250 mL). The combined organic layers were washed with water (400 mL), followed by brine (200 mL), dried over Na₂SO₄ then filtered and concentrated under reduced pressure. The material was purified by column chromatography (dry loading, EtOAc in hexanes, 0-5%, a gradient elution) to provide the title compound (4.18 g, 42%) as an oil. ¹H NMR (400 MHz, CDCl₃) δ 7.90-7.80 (m, 2H), 7.49 (s, 1H), 7.15-7.04 (m, 2H), 4.26 (t, J=6.8 Hz, 2H), 3.69 (t, J=5.7 Hz, 2H), 2.08 (p, J=5.8 Hz, 2H), 1.07-1.02 (m, 21H). m/z (ESI, +ve ion)=455.3, 457.3 [M+H]⁺.

### Step 4. 3-(4-Fluorophenyl)-4-(4,4,5,5-tetramethyl-1,3,2-dioxaborolan-2-yl)-1-(3-((triisopropylsilyl)oxy)propyl)-1H-pyrazole

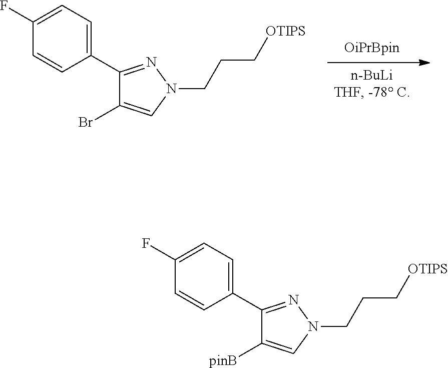

A solution of 4-Bromo-3-(4-fluorophenyl)-1-(3-((triisopropylsilyl)oxy)propyl)-1H-pyrazole (3.00 g, 6.59 mmol, 1.0 equiv.) in anhydrous THF (15 mL) in a flame-dried flask under nitrogen. was cooled to −78° C. To this solution was then added n-butyllithium (5.27 mL, 13.2 mmol, 2.0 equiv., 2.5 M in hexane) was added dropwise over 8 minutes. The reaction mixture was stirred at −78° C. for 1 hour, then 2-isopropoxy-4,4,5,5-tetramethyl-1,3,2-dioxaborolane (4.11 mL, 19.8 mmol, 3.0 equiv.) was added dropwise over 5 minutes. After 5 minutes, the reaction mixture was slowly warmed to RT over 16 hours. The reaction mixture was cooled to 0° C., then slowly diluted with a saturated aqueous solution of NH₄Cl (10 mL) and water (5 mL) and stirred vigorously for 5 minutes. The resulting mixture was diluted with EtOAc (20 mL) and water (20 mL), and the layers were separated. The aqueous layer was extracted with additional EtOAc (2×10 mL). The combined organic layers were washed with water (20 mL), dried over Na₂SO₄, filtered, and concentrated under reduced pressure to provide the title compound (4.41 g, quant.) as an oil. The material was used in the next steps without further purification. m/z (ESI, +ve ion)=503.5 [M+H]⁺.

### Intermediate G. 6-Iodo-8-phenyl-9H-purine

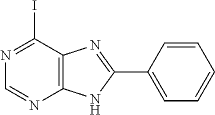 [SMILES: Ic1ncnc2[nH]c(-c3ccccc3)nc12]

### Step 1. 6-Chloro-8-phenyl-9H-purine

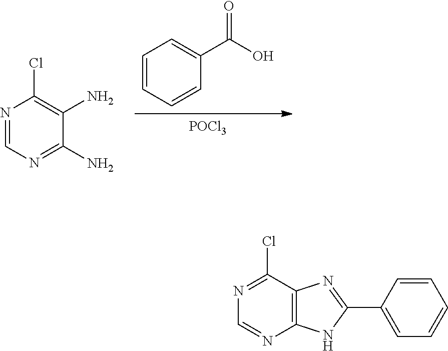

Phosphorus oxychloride (20.0 mL, 212 mmol, 63 equiv.) was added to a mixture of 6-chloropyrimidine-4,5-diamine (500 mg, 3.35 mmol), benzoic acid (412 mg, 3.35 mmol, 1.0 equiv.) and ammonium chloride (1.08 g, 20.1 mmol, 6.0 equiv.). The mixture was stirred for 24 h at 100° C., and then poured into an ice/water mixture. The pH was adjusted to 7-8 using an aqueous ammonium hydroxide solution (28-30%). The resulting solid was filtered and washed with water to provide the title compound (533 mg, 69%) as a solid. m/z (ESI, +ve ion)=231.1 [M+H]⁺.

### Step 2. 6-Iodo-8-phenyl-9H-purine

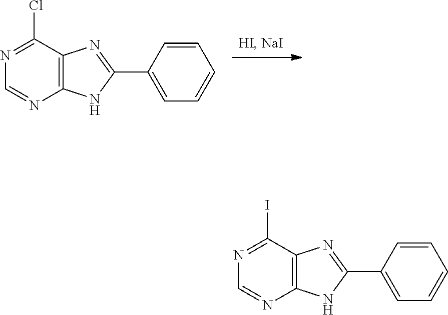

6-Chloro-8-phenyl-9H-purine (1.20 g, 5.18 mmol, 1.0 equiv.) and sodium iodide (1.05 g, 6.94 mmol, 1.3 equiv.) were suspended in hydriodic acid (10.0 mL, 44.0 mmol, 8.5 equiv.) and the resulting mixture was heated for 16 h at 60° C. The mixture was then cooled to RT and diluted with ice/water mixture (30 mL) and 2 M NaOH solution until the pH of the aqueous layer was between 7 to 8. The mixture was stirred vigorously until the ice melted. EtOAc (30 mL) and water (20 mL) were added, and the layers were separated. The aqueous layer was extracted with additional EtOAc (2×30 mL). The combined organic layers were washed with water (20 mL), dried over MgSO₄, filtered, and concentrated under reduced pressure to provide the title compound (1.20 g, 72%) as a solid. The crude material was used in the next step without further purification. m/z (ESI, +ve ion)=324.0 [M+H]⁺.

### Intermediate H. 7-Iodo-2-phenyloxazolo[5,4-d]pyrimidine

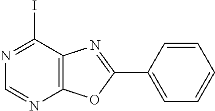 [SMILES: Ic1ncnc2oc(-c3ccccc3)nc12]

### Step 1. 7-Chloro-2-phenyloxazolo[5,4-d]pyrimidine

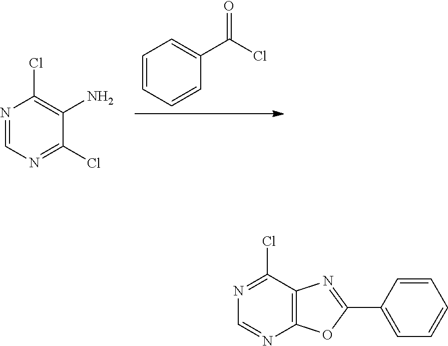 [SMILES: Nc1c(Cl)ncnc1Cl ; O=C(Cl)c1ccccc1 ; Clc1ncnc2oc(-c3ccccc3)nc12]

4,6-Dichloropyrimidin-5-amine (1.20 g, 7.17 mmol, 1.0 equiv) and benzoyl chloride (1.01 mL, 8.61 mmol, 1.2 equiv.) were heated in a microwave reactor at 110° C. for 2 hours. The residue was purified by column chromatography (MeOH in DCM, 0-4%, a gradient elution) to provide the title compound (860 mg, 52%) as a solid. ¹H NMR (400 MHz, CDCl₃) δ 8.81 (s, 1H), 8.36-8.28 (m, 2H), 7.70-7.62 (m, 1H), 7.61-7.53 (m, 2H). m/z (ESI, +ve ion)=232.0, 234.0 [M+H]⁺.

### Step 2. 7-Iodo-2-phenyloxazolo[5,4-d]pyrimidine

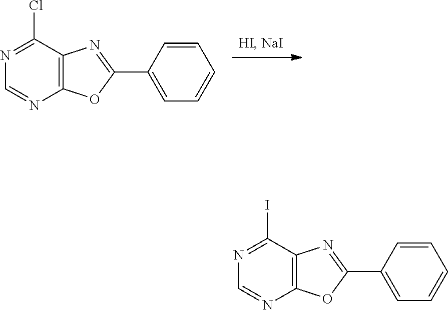

7-Chloro-2-phenyloxazolo[5,4-d]pyrimidine (830 mg, 3.58 mmol, 1.0 equiv) and sodium iodide (727 mg, 4.80 mmol, 1.3 equiv.) were suspended in hydriodic acid (3.22 mL, 18.0 mmol, 5.0 equiv.) and the resulting mixture was heated for 1 h at 60° C. The mixture was then cooled to RT and diluted with ice/water mixture (15 mL) and 2 M aq. NaOH solution (15 mL). The mixture was stirred vigorously until the ice melted. EtOAc (30 mL) and water (20 mL) were added, and the layers were separated. The aqueous layer was extracted with additional EtOAc (2×30 mL). The combined organic layers were washed with water (20 mL), dried over MgSO₄, filtered, and concentrated under reduced pressure to provide the title compound (1.05 g, 910%) as a solid. The crude material was used in the next step without further purification. ¹H NMR (400 MHz, CDCl₃) δ 8.70 (d, J=1.5 Hz, 1H), 8.36-8.31 (m, 2H), 7.66 (td, J=7.6, 1.5 Hz, 1H), 7.61-7.55 (m, 2H). m/z (ESI, +ve ion)=324.0 [M+H]⁺.

### Intermediate I. 4-Bromo-6-phenylfuro[2,3-d]pyrimidine

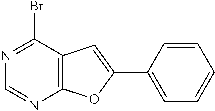 [SMILES: Brc1ncnc2oc(-c3ccccc3)cc12]

### Step 1. 6-Phenylfuro[2,3-d]pyrimidin-4 (3H)-one

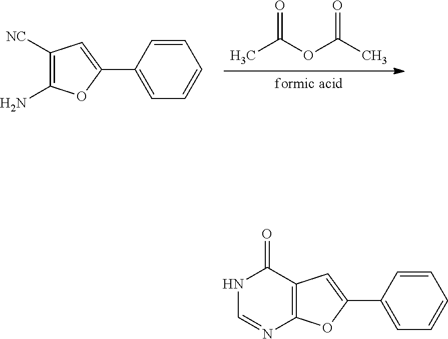

A mixture of 2-amino-5-phenylfuran-3-carbonitrile (25.0 g, 129 mmol, 1.0 equiv) in formic acid (404 mL, 10.3 mol, 80 equiv.) was cooled to 0° C. To this solution was added acetic anhydride (336 mL, 3.87 mol, 30 equiv.) dropwise over 1 hour, while maintaining the internal temperature under 5° C. After the completion of addition, the resulting mixture was warmed to RT over 1 hour then warmed to 85° C. for 60 hours. The reaction mixture was cooled to RT. The resulting precipitate was collected by suction filtration while rinsing with water (600 mL) to provide the title compound (22.2 g, 81%) as a solid. ¹H NMR (400 MHz, DMSO-d₆) δ 12.66 (s, 1H), 8.15 (s, 1H), 7.91-7.82 (m, 2H), 7.54-7.44 (m, 3H), 7.44-7.36 (m, 1H). m/z (ESI, +ve ion)=213.4 [M+H]⁺.

### Step 2. 4-Bromo-6-phenylfuro[2,3-d]pyrimidine

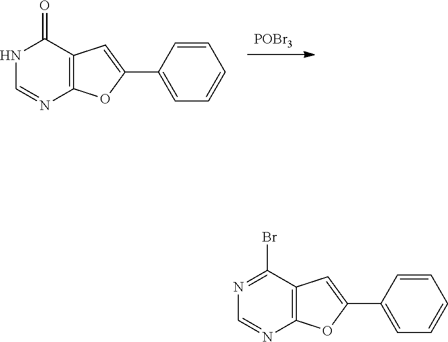 [SMILES: O=c1[nH]cnc2oc(-c3ccccc3)cc12 ; "POBr3" ; Brc1ncnc2oc(-c3ccccc3)cc12]

6-Phenylfuro[2,3-d]pyrimidin-4 (3H)-one (7.30 g, 34.4 mmol, 1.0 equiv) was added to phosphorus(V) oxybromide (125 g, 413 mmol, 12 equiv.) at 75° C. The mixture was stirred at 75° C. for 1.5 hours. Xylenes (21.9 mL) was added to the mixture at 75° C. The resulting solution was poured onto ice and the pH of the aqueous layer was adjusted to ˜ 6-7 using a solution of NH₄OH (30% ammonia in water) while maintaining the internal temperature below 10° C. The resulting precipitate was collected by suction filtration while washing with water. The filtrate was extracted with DCM (3×50 mL). The combined organic layers were washed with brine, then dried over Na₂SO₄, filtered, and concentrated under reduced pressure. The solids were combined to provide the title compound (9.40 g, 99%) as a solid. ¹H NMR (400 MHz, CDCl₃) δ 8.70 (s, 1H), 7.92 (dd, J=7.9, 1.5 Hz, 2H), 7.56-7.45 (m, 3H), 7.03 (s, 1H). m/z (ESI, +ve ion)=275.1, 277.1 [M+H]⁺.

### Intermediate J. 4-(3-(4-Fluorophenyl)-1-(3-((triisopropylsilyl)oxy)propyl)-1H-pyrazol-4-yl)-1-(phenylsulfonyl)-1H-pyrrolo[2,3-b]pyridine

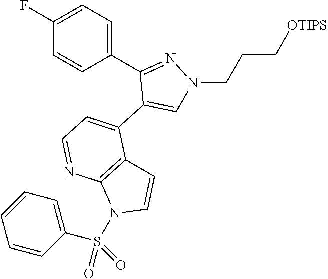

### Step 1. 4-Chloro-1-(phenylsulfonyl)-1H-pyrrolo[2,3-b]pyridine

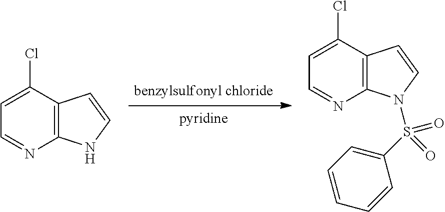 [SMILES: Clc1ccnc2[nH]ccc12 ; "benzylsulfonyl chloride" ; "pyridine" ; O=S(=O)(c1ccccc1)n1ccc2c(Cl)ccnc21]

Benzenesulfonyl chloride (6.6 mL, 51.4 mmol, 4 equiv.) was added to a mixture of 4-chloro-7-azaindole (2.0 g, 12.8 mmol, 1.0 equiv) in pyridine (14 mL). The mixture was stirred for 15 h and then cooled to 0° C. The mixture was diluted with water, and the resulting solid was filtered and washed with water to provide the title compound (3.40 g, 90%) as a solid. m/z (ESI, +ve ion)=293.0, 295.0 [M+H]⁺.

### Step 2. 4-Chloro-1-(phenylsulfonyl)-1H-pyrrolo[2,3-b]pyridine

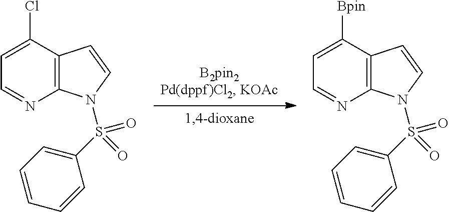

1,1′-Bis(diphenylphosphino)ferrocene dichloropalladium (II) (510 mg, 683 μmol, 0.1 equiv.) was added to a degassed mixture of 4-chloro-1-(phenylsulfonyl)-1H-pyrrolo[2,3-b]pyridine (2.00 g, 6.83 mmol), bis(pinacolato)diboron (1.79 g, 6.83 mmol, 1.0 equiv.), and potassium acetate (2.03 g, 20.5 mmol, 3.0 equiv.) in 1,4-dioxane (34.5 mL). The mixture was stirred under nitrogen at 95° C. for 4 h. The mixture was cooled to RT, diluted with EtOAc, and filtered through a pad of Celite, while washing with EtOAc, to provide (4.30 g, 164%) as an oil. The material was then directly used in the next step without further purification. m/z (ESI, +ve ion)=385.2 [M+H]⁺.

### Step 3. 4-(3-(4-Fluorophenyl)-1-(3-((triisopropylsilyl)oxy)propyl)-1H-pyrazol-4-yl)-1-(phenylsulfonyl)-1H-pyrrolo[2,3-b]pyridine

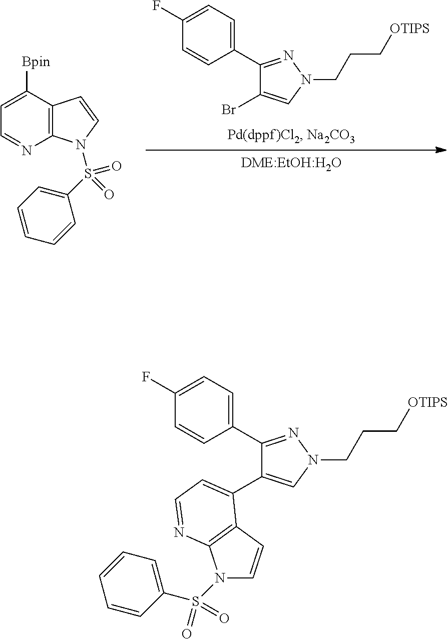

1,1′-Bis(diphenylphosphino)ferrocene dichloropalladium (II) (98.4 mg, 132 μmol, 0.05 equiv.) was added to a degassed mixture of sodium carbonate (846 mg, 7.90 mmol, 3.0 equiv.), 4-chloro-1-(phenylsulfonyl)-1H-pyrrolo[2,3-b]pyridine (1.52 g, 3.95 mmol, 1.5 equiv.) and 4-bromo-3-(4-fluorophenyl)-1-(3-((triisopropylsilyl)oxy)propyl)-1H-pyrazole (Product of Step 3 of Intermediate F, 1.20 g, 2.63 mmol) in a mixture of DME (13.5 mL), EtOH (7.76 mL) and H₂O (3.37 mL). The mixture was stirred for 15 h at 100° C., cooled to RT and diluted with water (30 mL). The aqueous layer was extracted with EtOAc (3×40 mL). The combined organic layers were washed with brine and concentrated under reduced pressure. The residue was purified by column chromatography (EtOAc in hexanes, 0-20%, a gradient elution) to provide the title compound (820 mg, 49%) as a solid. m/z (ESI, +ve ion)=633.1 [M+H]⁺.

### Intermediate K. 3-(4-Fluorophenyl)-4-(4,4,5,5-tetramethyl-1,3,2-dioxaborolan-2-yl)-1-((2-(trimethylsilyl)ethoxy)methyl)-1H-pyrazole and 5-(4-fluorophenyl)-4-(4,4,5,5-tetramethyl-1,3,2-dioxaborolan-2-yl)-1-((2-(trimethylsilyl)ethoxy)methyl)-1H-pyrazole

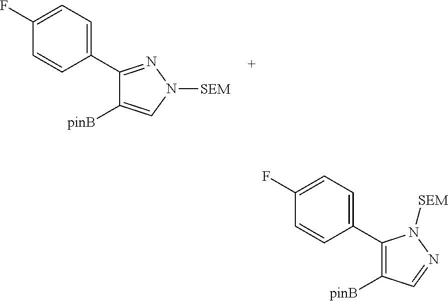

### Step 1. 4-Bromo-3-(4-fluorophenyl)-1-((2-(trimethylsilyl)ethoxy)methyl)-1H-pyrazole and 4-bromo-5-(4-fluorophenyl)-1-((2-(trimethylsilyl)ethoxy)methyl)-1H-pyrazole

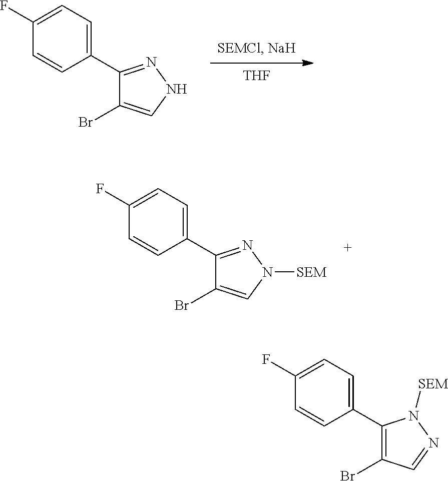

Sodium hydride (3.98 g, 99.6 mmol, 1.2 equiv, 60% in dispersion in mineral oil) was slowly added to a solution of 4-bromo-3-(4-fluorophenyl)-1H-pyrazole (20.0 g, 83.0 mmol, 1.0 equiv) in THF (300 mL) at 0° C. The mixture was stirred for 1 h and then (2-chloromethoxyethyl)trimethylsilane (16.5 mL, 83.8 mmol, 1.01 equiv) was added. The mixture was stirred for 16 h at RT then diluted with water (400 mL) and extracted with EtOAc (3×300 mL). The combined organic layers were washed with water, brine, dried over Na₂SO₄, filtered then concentrated to provide the title compounds (as a mixture of N-1 and N-2 alkylated regioisomers) (33.6 g, quant.) as an oil. The material was used in the next steps without further purification. ¹H NMR (400 MHz, CDCl₃) δ 7.87 (dd, J=9.0, 5.4 Hz, 2H), 7.67 (s, 1H), 7.62-7.54 (m, 3H), 7.19 (t, J=8.7 Hz, 2H), 7.12 (t, J=8.8 Hz, 2H), 5.42 (s, 2H), 5.31 (s, 2H), 3.74-3.65 (m, 2H), 3.62 (dd, J=5.2, 4.0 Hz, 2H), 0.96-0.89 (m, 4H), −0.00 (s, 9H), −0.01 (s, 9H) (reported as a 1:1 mixture of N-1 and N-2 alkylated regioisomers). m/z (ESI, +ve ion)=371.2, 373.2 [M+H]⁺.

### Step 2. 3-(4-Fluorophenyl)-4-(4,4,5,5-tetramethyl-1,3,2-dioxaborolan-2-yl)-1-((2-(trimethylsilyl)ethoxy)methyl)-1H-pyrazole

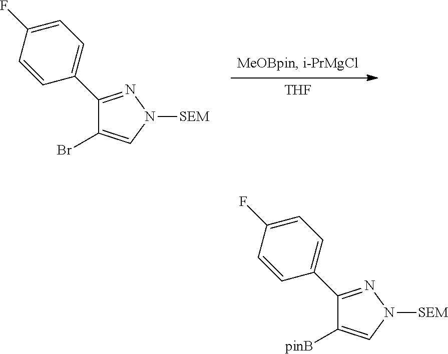

Isopropylmagnesium chloride (9.70 mL, 19.4 mmol, 1.6 equiv) was added to a mixture of 4-bromo-3-(4-fluorophenyl)-1-((2-(trimethylsilyl)ethoxy)methyl)-1H-pyrazole (4.50 g, 12.1 mmol, 1.0 equiv, as a mixture of N-1 and N-2 alkylated regioisomers) in THF (30.2 mL). The mixture was stirred for 8.5 h at RT. At this point, the solution was then cooled to at 0° C. Once cooled, 2-methoxy-4,4,5,5-tetramethyl-1,3,2-dioxaborolane (4.05 mL, 24.2 mmol, 2.0 equiv) was slowly added to the solution. The mixture was stirred for 1 h at 0° C. and then allowed to stir for 15 h at RT. The mixture was diluted with saturated aqueous NH₄Cl (150 mL) and water (150 mL) and extracted with EtOAc (3×100 mL). The combined organic layers were washed with brine, dried (Na₂SO₄), filtered and then concentrated under reduced pressure. The residue was purified by column chromatography (EtOAc in hexanes, 0-20%, a gradient elution) to provide the title compounds (as a mixture of N-1 and N-2 alkylated regioisomers) (4.20 g, 83%) as an oil. m/z (ESI, +ve ion)=419.4 [M+H]⁺.

### Intermediate L. 7-(3-(4-Fluorophenyl)-1H-pyrazol-4-yl)-2-phenyloxazolo[5,4-d]pyrimidine

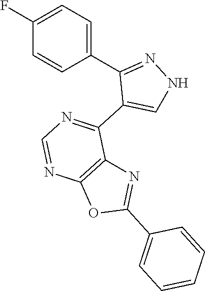 [SMILES: Fc1ccc(-c2n[nH]cc2-c2ncnc3oc(-c4ccccc4)nc23)cc1]

### Step 1. 7-(3-(4-Fluorophenyl)-1-((2-(trimethylsilyl)ethoxy)methyl)-1H-pyrazol-4-yl)-2-phenyloxazolo[5,4-d]pyrimidine

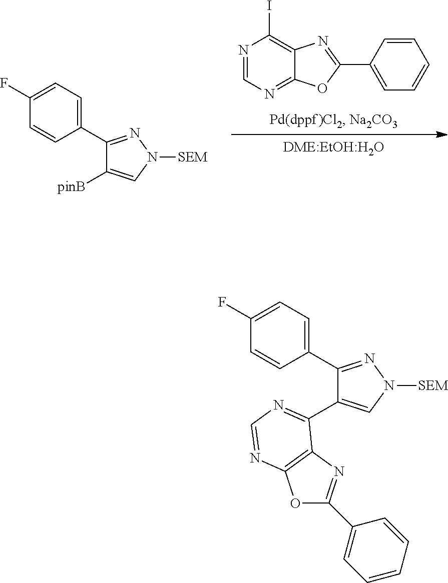

1,1′-Bis(diphenylphosphino)ferrocene dichloropalladium (II) (5.78 mg, 7.74 μmol, 0.1 equiv) was added to a degassed mixture of sodium carbonate (24.9 mg, 232 μmol, 3.0 equiv), 3-(4-fluorophenyl)-4-(4,4,5,5-tetramethyl-1,3,2-dioxaborolan-2-yl)-1-((2-(trimethylsilyl)ethoxy)methyl)-1H-pyrazole (Intermediate K, 48.6 mg, 116 μmol, 1.5 equiv, as a mixture of N-1 and N-2 alkylated regioisomers) and 7-iodo-2-phenyloxazolo[5,4-d]pyrimidine (Intermediate H, 25.0 mg, 77.4 μmol) in a mixture of DME (396 μL), EtOH (228 μL) and H₂O (99.1 μL). The mixture was stirred for 30 minutes at 110° C. in a microwave reactor then directly loaded on silica and purified by column chromatography (EtOAc in hexanes, 0-20%, a gradient elution) to provide the title compound (as a mixture of N-1 and N-2 alkylated regioisomers) (14.0 mg, 37%) as a solid. m/z (ESI, +ve ion)=488.2 [M+H]⁺.

### Step 4. 7-(3-(4-Fluorophenyl)-1H-pyrazol-4-yl)-2-phenyloxazolo[5,4-d]pyrimidine

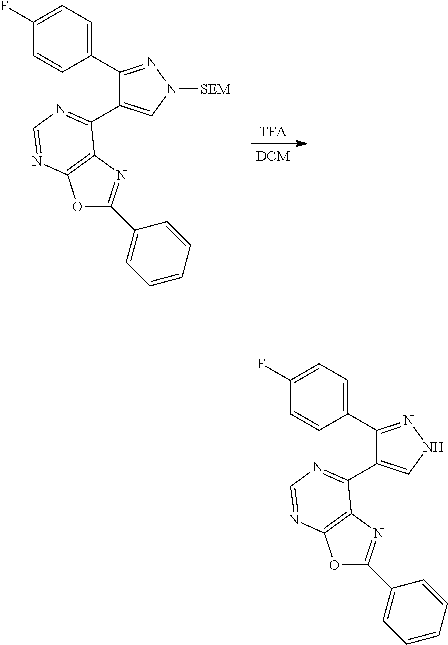

Trifluoroacetic acid (1.74 mL, 22.6 mmol, 20 equiv) was added to a mixture of 7-(3-(4-fluorophenyl)-1-((2-(trimethylsilyl)ethoxy)methyl)-1H-pyrazol-4-yl)-2-phenyloxazolo[5,4-d]pyrimidine (550 mg, 1.13 mmol, as a mixture of N-1 and N-2 alkylated regioisomers) in DCM (9.82 mL). The mixture was stirred for 15 h at RT. The pH was adjusted at 7 using saturated aqueous solution NaHCO₃. The resulting solid was filtered and washed with water and DCM. The solid was diluted with MeOH and concentrated under reduced pressure to provide the title compound (430 mg, quant.) as a solid. m/z (ESI, +ve ion)=358.2 [M+H]⁺.

### Intermediate M. 4-Bromo-5-(4-fluorophenyl)-2H-1,2,3-triazole

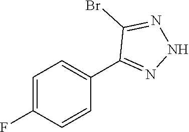 [SMILES: Fc1ccc(-c2n[nH]nc2Br)cc1]

### Step 1. 4-(4-Fluorophenyl)-2H-1,2,3-triazole

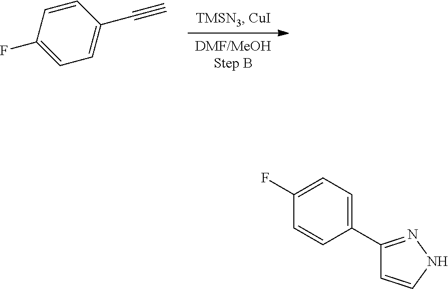

To a solution of 1-ethynyl-4-fluorobenzene (1.2 g, 10.0 mmol, 1 equiv) and azidotrimethylsilane (1.73 g, 15.0 mmol, 1.5 equiv) in MeOH (2 mL) and DMF (10 mL) was added CuI (0.10 g, 0.50 mmol, 0.05 equiv) at RT under nitrogen atmosphere. The resulting mixture was stirred for 16 h at 80° C. under nitrogen atmosphere then cooled down to RT. The resulting mixture was diluted with water (10 mL) and extracted with CHCl₃ (3×20 mL). The combined organic layers were washed with brine (2×7 mL), dried over anhydrous Na₂SO₄. After filtration, the filtrate was concentrated under reduced pressure. The residue was purified by silica gel column chromatography, eluted with DCM:EtOAc (1:1) to afford the title compound (1.3 g, 80%) as an off-white solid. m/z (ESI, +ve ion)=164.05 [M+H]⁺.

### Step 2. 4-Bromo-5-(4-fluorophenyl)-2H-1,2,3-triazole

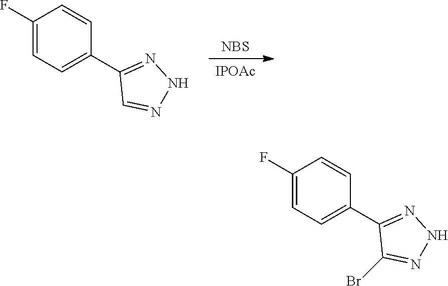 [SMILES: Fc1ccc(-c2cn[nH]n2)cc1 ; "NBS" ; Fc1ccc(-c2n[nH]nc2Br)cc1 ; "IPOAc"]

To a solution of 4-(4-fluorophenyl)-2H-1,2,3-triazole (1.2 g, 7.36 mmol, 1 equiv) in propan-2-yl acetate (12 mL) was added NBS (1.31 g, 7.36 mmol, 1.0 equiv) at RT under nitrogen atmosphere. The resulting mixture was stirred for 5 h at RT. The resulting mixture was filtered and the filter cake was washed with ethyl acetate (2×4 mL). The filtrate was concentrated under reduced pressure to afford the title compound (1.3 g, 73%) as a white solid. m/z (ESI+ve ion)=242.05, 244.05 [M+H]⁺.

### Intermediate N. 4-[3-(4-Fluorophenyl)-1H-pyrazol-4-yl]-6-phenylfuro[2,3-d]pyrimidine

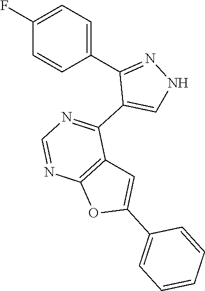 [SMILES: Fc1ccc(-c2n[nH]cc2-c2ncnc3oc(-c4ccccc4)cc23)cc1]

### Step 1. 4-(3-(4-Fluorophenyl)-1-((2-(trimethylsilyl)ethoxy)methyl)-1H-pyrazol-4-yl)-6-phenylfuro[2,3-d]pyrimidine

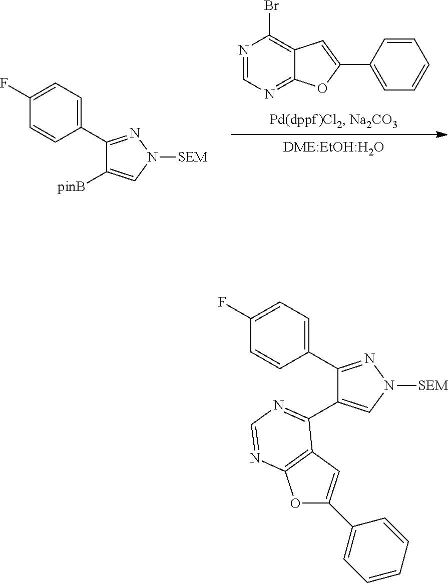

1,1′-Bis(diphenylphosphino)ferrocene dichloropalladium (II) (1.39 g, 1.86 mmol, 0.1 equiv) was added to a degassed mixture of 3-(4-fluorophenyl)-4-(4,4,5,5-tetramethyl-1,3,2-dioxaborolan-2-yl)-1-((2-(trimethylsilyl)ethoxy)methyl)-1H-pyrazole (Intermediate K, 10.4 g, 18.6 mmol, as a mixture of N-1 and N-2 alkylated regioisomers), 4-bromo-6-phenylfuro[2,3-d]pyrimidine (Intermediate I, 5.54 g, 20.1 mmol, 1.1 equiv) and sodium carbonate (5.99 g, 55.9 mmol, 3.0 equiv) in a mixture of DME (122 mL), EtOH (25.7 mL) and H₂O (17.1 mL). The reaction mixture was stirred for 16 hours at 95° C. under positive nitrogen pressure. The mixture was cooled and then concentrated under reduced pressure until ⅓ of its original volume. The reaction mixture was diluted with water (400 mL) and EtOAc (125 mL). The layers were separated, and the aqueous layer was extracted with additional EtOAc (2×150 mL). The combined organic layers were washed with brine, dried over Na₂SO₄, filtered, and concentrated under reduced pressure. The residue was purified by column chromatography (EtOAc in hexanes, 0-50%, a gradient elution), to provide the title compound (7.72 g, 80%, as a mixture of N-1 and N-2 alkylated regioisomers) as an oil. m/z (ESI, +ve ion)=487.4 [M+H]⁺.

### Step B. 4-[3-(4-Fluorophenyl)-1H-pyrazol-4-yl]-6-phenylfuro[2,3-d]pyrimidine

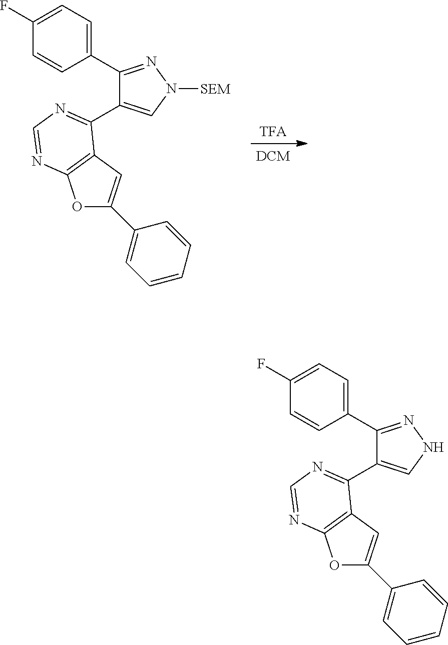

Trifluoroacetic acid (9.70 mL, 125 mmol, 10 equiv) was added dropwise to a solution of 4-(3-(4-fluorophenyl)-1-((2-(trimethylsilyl)ethoxy)methyl)-1H-pyrazol-4-yl)-6-phenylfuro[2,3-d]pyrimidine (6.10 g, 12.5 mmol, as a mixture of N-1 and N-2 alkylated regioisomers) in DCM (83.6 mL) at 0° C. The mixture was warmed to RT over 16 h, then diluted with a solution of ammonium hydroxide (90 mL, 28-30% NH₃ in water). The resulting mixture was vigorously stirred until precipitate formation was observed. The resulting precipitate was collected by suction filtration while washing with water. The resulting filtrate was extracted with additional DCM (30 mL) and EtOAc (2×20 mL). The combined organic layers were stirred in a solution of ammonium hydroxide (25 mL, 28-30% NH₃ in water) for 1 h, then concentrated under reduced pressure. The resulting solids were combined and triturated in minimal MeOH, then collected by suction filtration while washing successively with MeOH, DCM followed by MeCN. This process was repeated numerous times to provide the title compound (4.38 g, 95%) as a solid. ¹H NMR (400 MHz, DMSO-d₆) δ 13.60 (br s, 1H), 8.75 (s, 1H), 8.51 (s, 1H), 7.94 (d, J=7.4 Hz, 2H), 7.62 (dd, J=8.5, 5.7 Hz, 2H), 7.55 (t, J=7.4 Hz, 2H), 7.52-7.50 (m, 1H), 7.47 (d, J=7.4 Hz, 1H), 7.31-7.16 (m, 1H). m/z (ESI, +ve ion)=357.3 [M+H]⁺.

### Intermediate O. Methyl 3-[5-bromo-4-(4-fluorophenyl)-1,3-oxazol-2-yl]propanoate

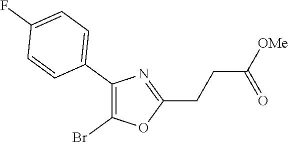 [SMILES: COC(=O)CCc1nc(-c2ccc(F)cc2)c(Br)o1]

### Step 1. Methyl 3-[4-(4-fluorophenyl)-1,3-oxazol-2-yl]propanoate

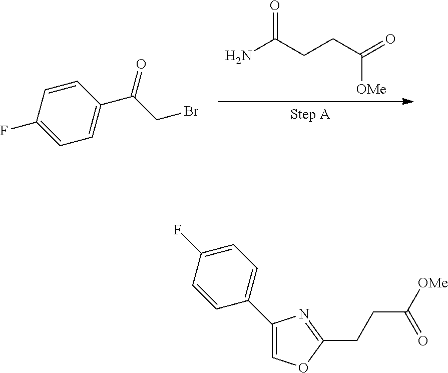 [SMILES: O=C(CBr)c1ccc(F)cc1 ; COC(=O)CCC(N)=O ; "Step A" ; COC(=O)CCc1nc(-c2ccc(F)cc2)co1]

Into a 20 mL pressure tank reactor were added 2-bromo-1-(4-fluorophenyl)ethanone (1.7 g, 7.83 mmol, 1 equiv) and methyl 3-carbamoylpropanoate (2.05 g, 15.7 mmol, 2 equiv). The mixture was stirred at 140° C. for 6 h under nitrogen atmosphere. The reaction was quenched by the addition of water (100 mL) at RT. The resulting mixture was extracted with EtOAc (3×100 mL). The combined organic layers were washed with brine (100 mL), dried over anhydrous Na₂SO₄ filtered, and then concentrated under reduced pressure. The residue was purified by silica gel column chromatography, eluted with PE:EA (1:1) to afford the title compound (660 mg, 34%) as a yellow solid. m/z (ESI, +ve ion)=250.05 [M+H]⁺.

### Step 2. Methyl 3-[5-bromo-4-(4-fluorophenyl)-1,3-oxazol-2-yl]propanoate

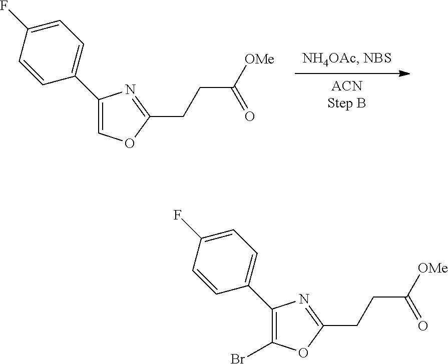

To a stirred solution of methyl 3-[4-(4-fluorophenyl)-1,3-oxazol-2-yl]propanoate (610 mg, 2.45 mmol, 1 equiv) and NH₄OAc (18.9 mg, 0.245 mmol, 0.1 equiv) in ACN (7 mL) was added NBS (436 mg, 2.45 mmol, 1 equiv) in portions at RT under nitrogen atmosphere. The resulting mixture was stirred for 2 h at RT under nitrogen atmosphere. The reaction was quenched by the addition of water (50 mL) at RT. The resulting mixture was extracted with EtOAc (3×50 mL). The combined organic layers were washed with brine (50 mL), dried over anhydrous Na₂SO₄ filtered and concentrated under reduced pressure. The residue was purified by silica gel column chromatography, eluted with PE:EtOAc (3:1) to afford the title compound (550 mg, 69%) as a yellow solid. m/z (ESI, +ve ion)=328.10, 330.10 [M+H]⁺.

### Intermediate P. 1-(3-Bromo-4-(6-phenylfuro[2,3-d]pyrimidin-4-yl)-1H-pyrazol-1-yl)-2-methylpropan-2-ol

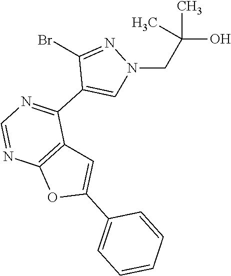 [SMILES: CC(C)(O)Cn1cc(-c2ncnc3oc(-c4ccccc4)cc23)c(Br)n1]

### Step 1. 3-Bromo-4-iodo-1H-pyrazole

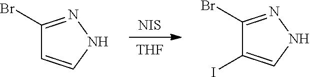

N-iodosuccimmide 26.4 g, 115 mmol, 1.0 equiv. was added to a solution o 3-bromopyrazole (17.8 g, 115 mmol, 1.0 equiv.) in THF (523 mL). The reaction mixture was stirred at RT for 20 h. The reaction mixture was concentrated, then diluted with EtOAc (200 mL) and water. The organic layer was washed with a saturated solution of Na₂S₂O₃, dried over Na₂SO₄, filtered, and concentrated to afford the title compound (30.8 g, 86%) as a solid. The material was used in the next steps without further purification. ¹H NMR (400 MHz, DMSO-d₆) δ 13.49 (s, 1H), 7.97 (d, J=1.4 Hz, 1H). m/z (ESI, +ve ion)=272.9, 274.9 [M+H]⁺.

### Step 2. 3-Bromo-4-iodo-1-((2-(trimethylsilyl)ethoxy)methyl)-1H-pyrazole and 5-bromo-4-iodo-1-((2-(trimethylsilyl)ethoxy)methyl)-1H-pyrazole

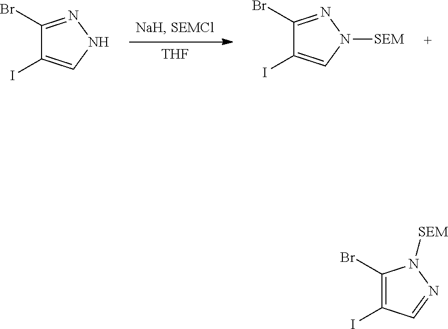

Sodium hydride (3.22 g, 80.5 mmol, 1.2 equiv., 60% in dispersion in mineral oil) was added to a solution of 3-bromo-4-iodo-1H-pyrazole (20.8 g, 67.1 mmol, 1.0 equiv) and THF (243 mL) at 0° C. The mixture was stirred at 0° C. for 1 h and then (2-chloromethoxyethyl)trimethylsilane (13.2 mL, 67.1 mmol, 1.0 equiv.) was added. The reaction mixture was warmed to RT over 2 h. The mixture was diluted with water and extracted with EtOAc (3×70 mL). The combined organic layers were washed with brine, dried over Na₂SO₄, filtered and concentrated. The residue was purified by column chromatography (EtOAc in hexanes, 0-10%, a gradient elution) to afford the title compound (as a mixture of regioisomers) (27.1 g, quant.) as an oil. m/z (ESI, +ve ion)=403.0, 405.0 [M+H]⁺.

### Step 3. 3-Bromo-4-(4,4,5,5-tetramethyl-1,3,2-dioxaborolan-2-yl)-1-((2-(trimethylsilyl)ethoxy)methyl)-1H-pyrazole

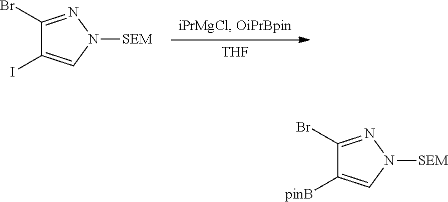

3-Bromo-4-iodo-1-((2-(trimethylsilyl)ethoxy)methyl)-1H-pyrazole (5.00 g, 12.4 mmol, as a mixture of regioisomers) was added into the reaction vessel and purged with nitrogen and evacuated three times. THF (30.9 mL) was added and then isopropylmagnesium chloride (9.30 mL, 18.6 mmol, 1.5 equiv.) was added dropwise at RT. The mixture was stirred at RT for 1 h, then 2-methoxy-4,4,5,5-tetramethyl-1,3,2-dioxaborolane (4.09 mL, 24.4 mmol, 2.0 equiv.) was added and the mixture was further stirred at RT for 50 minutes. The mixture was diluted with saturated aqueous ammonium chloride solution and partitioned between water (200 mL) and EtOAc (200 mL). The aqueous layer was further extracted with EtOAc (200 mL×2). The organic phase was dried over Na₂SO₄ and concentrated under reduced pressure. The residue was passed through a pad of silica while washing with EtOAc. The filtrated was concentrated under reduced pressure to provide the title compound (as a mixture of regioisomers) (5.17 g, quant.) as an oil. m/z (ESI, +ve ion)=403.2, 405.2 [M+H]⁺.

### Step 4. 4-(3-Bromo-1-((2-(trimethylsilyl)ethoxy)methyl)-1H-pyrazol-4-yl)-6-phenylfuro[2,3-d]pyrimidine

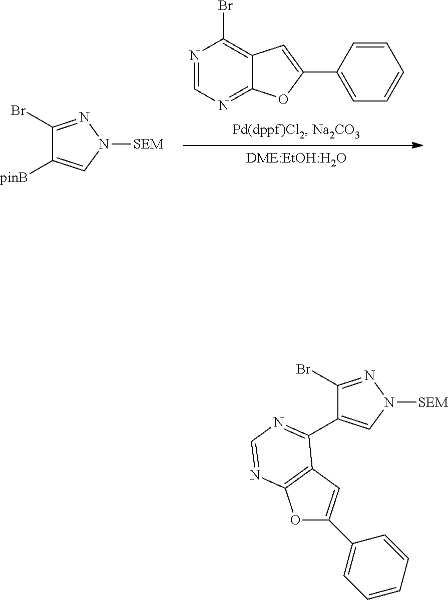

4-Bromo-6-phenylfuro[2,3-d]pyrimidine (Intermediate I, 7.49 g, 27.2 mmol, 1.2 equiv.), 3-bromo-4-(4,4,5,5-tetramethyl-1,3,2-dioxaborolan-2-yl)-1-((2-(trimethylsilyl)ethoxy)methyl)-1H-pyrazole (12.1 g, 22.5 mmol, 1.0 equiv, as a mixture of regioisomers), 1,1′-bis(diphenylphosphino)ferrocene dichloropalladium (II) (1.68 g, 2.25 mmol, 0.1 equiv.) and sodium carbonate (7.23 g, 67.5 mmol, 3.0 equiv.) were dissolved in DME (147 mL), EtOH (31.0 mL) and H₂O (20.7 mL). The flask was purged with nitrogen and evacuated three times. The mixture was stirred for 20 h at 80° C. then cooled to RT. The mixture was diluted with water and extracted with EtOAc (3×200 mL). The combined organic layers were washed with brine, dried over Na₂SO₄, filtered and concentrated. The residue was purified by column chromatography (EtOAc in hexane, 0-50%, a gradient elution) to provide the title compound (as a mixture of regioisomers) (3.97 g, 37%) as an oil. m/z (ESI, +ve ion)=471.3, 473.3 [M+H]⁺.

### Step 5. 4-(3-Bromo-1H-pyrazol-4-yl)-6-phenylfuro[2,3-d]pyrimidine

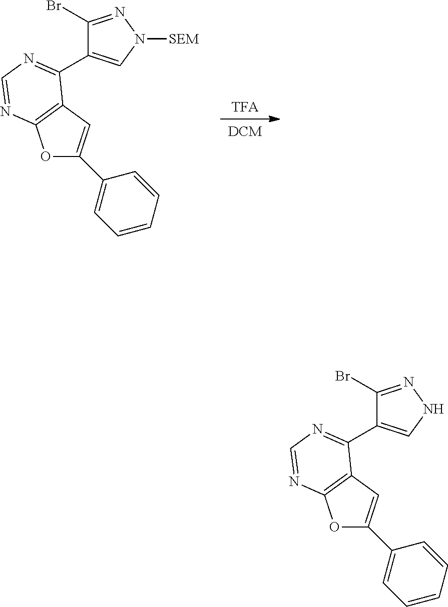

4-(3-Bromo-1-((2-(trimethylsilyl)ethoxy)methyl)-1H-pyrazol-4-yl)-6-phenylfuro[2,3-d]pyrimidine (1.30 g, 2.76 mmol, as a mixture of regioisomers) was dissolved in DCM (24.0 mL), then trifluoroacetic acid (4.27 mL, 55.2 mmol, 20 equiv.) was added to the solution and stirred at RT for 18 h. The reaction mixture was concentrated under reduced pressure until half of its original volume. The pH of the mixture was adjusted to ˜7 using a solution of ammonium hydroxide (30% solution of NH₃ in water) and the aqueous mixture was extracted with EtOAc (30 mL). The aqueous layer was further extracted with EtOAc (20 mL×2). The organic layers were combined and the solvent was evaporated under reduced pressure. The residue was triturated in minimal amounts of MeOH then diethyl ether was added until precipitate formation was observed. The precipitate was collected by suction filtration to provide the title compound (611 mg, 65%) as a solid. m/z (ESI, +ve ion)=341.1, 343.1 [M+H]⁺.

### Step 6. 1-(3-Bromo-4-(6-phenylfuro[2,3-d]pyrimidin-4-yl)-1H-pyrazol-1-yl)-2-methylpropan-2-ol

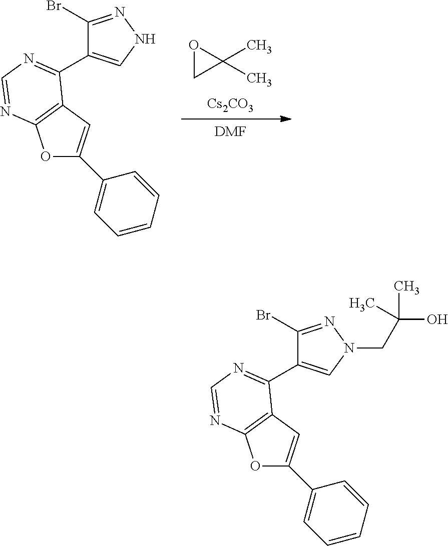

4-(3-Bromo-1H-pyrazol-4-yl)-6-phenylfuro[2,3-d]pyrimidine (2.15 g, 6.30 mmol, 1.0 equiv) and cesium carbonate (5.19 g, 15.8 mmol, 2.5 equiv.) were suspended in DMF (63 mL). The mixture was stirred for 30 minutes at RT, then 1,2-epoxy-2-methylpropane (1.73 mL, 18.9 mmol, 3.0 equiv.) was added. The mixture was heated under reflux at 90° C. for 5 h. The reaction mixture was cooled to RT and diluted with water (50 mL). The mixture was extracted with EtOAc (3×50 mL). The combined organic layers were washed with brine, then dried over Na₂SO₄, filtered and concentrated. The residue was purified by column chromatography (EtOAc in hexane, 0-100%, a gradient elution) and then by reverse phase chromatography (C18 column, acetonitrile in water with 10 mM ammonium formate, 5-70%) to provide the title compound (1.20 g, 45%) as a solid. ¹H NMR (400 MHz, DMSO-d₆) δ 8.94 (s, 1H), 8.63 (s, 1H), 8.04-8.00 (m, 2H), 7.85 (s, 1H), 7.61-7.56 (m, 2H), 7.54-7.49 (m, 1H), 4.87 (s, 1H), 4.16 (s, 2H), 1.16 (s, 6H). m/z (ESI, +ve ion)=413.2, 415.2 [M+H]⁺.

### Intermediate Q. 6-Bromo-4-(3-(4-fluorophenyl)-1H-pyrazol-4-yl)furo[2,3-d]pyrimidine

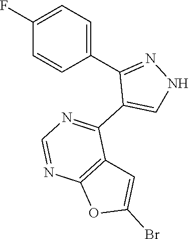 [SMILES: Fc1ccc(-c2n[nH]cc2-c2ncnc3oc(Br)cc23)cc1]

### Step A. 4-(3-(4-Fluorophenyl)-1-((2-(trimethylsilyl)ethoxy)methyl)-1H-pyrazol-4-yl)furo[2,3-d]pyrimidine

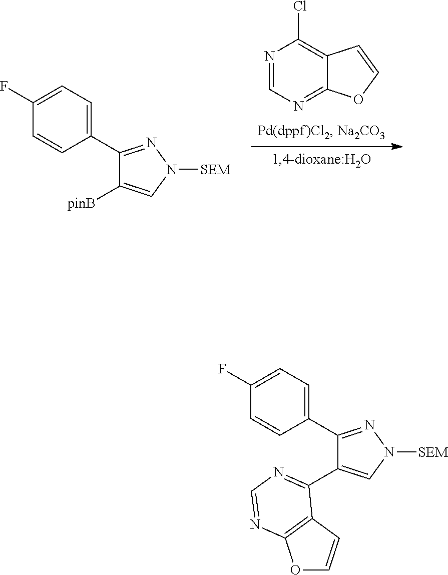

1,1′-Bis(diphenylphosphino)ferrocene dichloropalladium (II) (249 mg, 324 μmol, 0.1 equiv.) was added to a degassed mixture of sodium carbonate (1.04 g, 9.71 mmol, 3.0 equiv.), 3-(4-fluorophenyl)-4-(4,4,5,5-tetramethyl-1,3,2-dioxaborolan-2-yl)-1-((2-(trimethylsilyl)ethoxy)methyl)-1H-pyrazole (Intermediate K, 1.62 g, 3.88 mmol, 1.2 equiv., as a mixture of regioisomers) and 4-chlorofuro[2,3-d]pyrimidine (500 mg, 3.24 mmol, 1.0 equiv) were suspended in a mixture of 1,4-dioxane (20 mL) and H₂O (0.75 mL). The mixture was further degassed for 5 minutes using nitrogen, then heated at 80° C. for 20 h. After cooling to RT, the mixture was filtered through a pad of Celite and eluted with EtOAc and the filtrate was concentrated under reduced pressure. The residue was purified by column chromatography (EtOAc in hexanes, a gradient of 0-20%) to provide the title compound (840 mg, 63%, a mixture of regioisomers) as an oil. m/z (ESI, +ve ion)=411.4 [M+H]⁺.

### Step B. 4-(3-(4-Fluorophenyl)-1H-pyrazol-4-yl)furo[2,3-d]pyrimidine

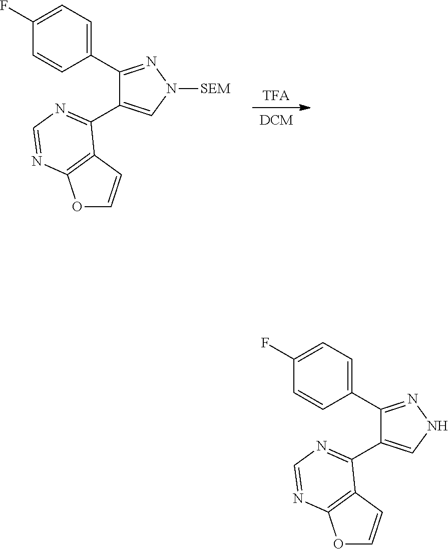

Trifluoroacetic acid (1.65 mL, 21.3 mmol, 5.0 equiv.) was added to a mixture of 4-(3-(4-fluorophenyl)-1-((2-(trimethylsilyl)ethoxy)methyl)-1H-pyrazol-4-yl)furo[2,3-d]pyrimidine (1.75 g, 4.26 mmol, as a mixture of regioisomers) in DCM (30.0 mL). The mixture was stirred for 16 h at RT. The volatiles were removed under reduced pressure, then the pH was adjusted to 7-8 using 15% w/w NaOH solution in water. The aqueous layer was extracted with EtOAc (3×20 mL) and the combined organic layers were washed with brine (15 mL), dried over Na₂SO₄, filtered, and concentrated. The residue was purified by column chromatography (EtOAc in hexanes, 0-100%, a gradient elution) to provide the title compound (910 mg, 76%) as a solid. m/z (ESI, +ve ion)=281.1 [M+H]⁺.

### Step C. 6-Bromo-4-(3-(4-fluorophenyl)-1H-pyrazol-4-yl)furo[2,3-d]pyrimidine

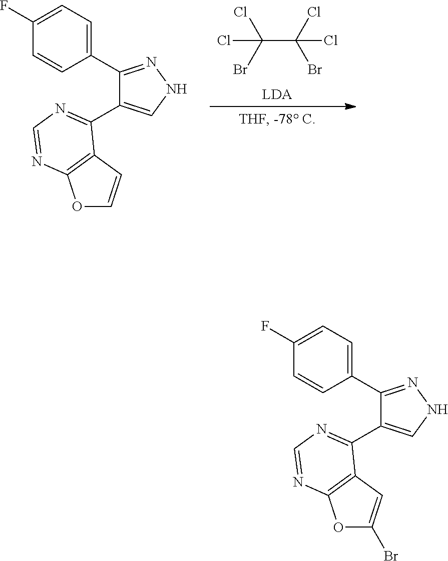

4-(3-(4-Fluorophenyl)-1H-pyrazol-4-yl)furo[2,3-d]pyrimidine (910 mg, 3.25 mmol) was dissolved in THF (34.6 mL) and the mixture was cooled down to 0° C. Sodium hydride (260 mg, 6.49 mmol, 2.0 equiv., 60% in dispersion in mineral oil) was added under nitrogen and the mixture was stirred at 0° C. for 30 minutes and then cooled to −78° C. Lithium diisopropylamide (2.44 mL, 4.87 mmol, 1.5 equiv.) was added dropwise and the mixture was stirred at −78° C. for 1 h, after which a solution of 1,2-dibromotetrachloroethane (1.64 g, 4.87 mmol, 1.5 equiv.) in THF (11.5 mL) was added dropwise. The mixture was further stirred at −78° C. for 3 h. The reaction mixture was slowly diluted with H₂O (15 mL) then warmed to RT. A saturated solution of NH₄Cl (10 mL) and EtOAc (15 mL) were added. The aqueous layer was extracted with EtOAc (3×25 mL). The combined organic layers were washed with Na₂S₂O₃ (10 mL), H₂O (10 mL), brine (10 mL), then dried over Na₂SO₄, filtered, and concentrated under reduced pressure. The residue was purified by column chromatography (EtOAc in hexanes, a gradient of 10-100%) to provide the title compound (0.720 g, 62%) as a solid. m/z (ESI, +ve ion)=359.0, 361.0 [M+H]⁺.

### Intermediate R. 6-(3-(4-Fluorophenyl)-1H-pyrazol-4-yl)-9-(4-methoxybenzyl)-8-phenyl-9H-purine

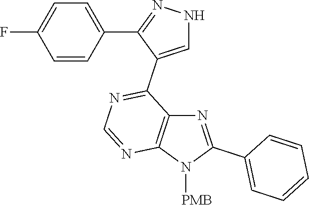

### Step 1. 6-(3-(4-Fluorophenyl)-1-((2-(trimethylsilyl)ethoxy)methyl)-1H-pyrazol-4-yl)-8-phenyl-9H-purine

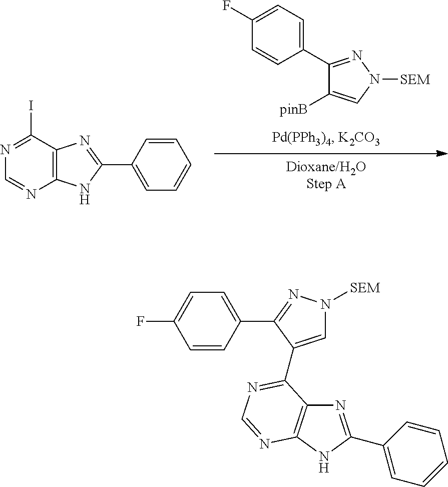

To a stirred mixture of 6-iodo-8-phenyl-9H-purine (Intermediate G, 1.28 g, 3.974 mmol, 1 equiv) and 3-(4-fluorophenyl)-4-(4,4,5,5-tetramethyl-1,3,2-dioxaborolan-2-yl)-1-{[2-(trimethylsilyl)ethoxy]methyl}pyrazole (Intermediate K, 2.49 g, 5.96 mmol, 1.5 equiv, as a mixture of regioisomers) in dioxane (20 mL) and water (4 mL) were added Pd(PPh₃)₄ (0.23 g, 0.12 mmol, 0.05 equiv) and K₂CO₃ (1.65 g, 11.9 mmol, 3 equiv) at RT under nitrogen atmosphere. The resulting mixture was stirred for 16 h at 110° C. under nitrogen atmosphere then quenched by the addition of water (20 mL) at RT. The resulting mixture was extracted with EtOAc (3×30 mL). The combined organic layers were washed with brine (90 mL), dried over anhydrous Na₂SO₄. After filtration, the filtrate was concentrated under reduced pressure. The residue was purified by silica gel column chromatography, eluted with PE:EA (0:1) to afford the title compound (1.26 g, 63%) as a white solid (as a mixture of regioisomers). m/z (ESI, +ve ion)=487.15 [M+H]⁺.

### Step 2. 6-(3-(4-Fluorophenyl)-1-((2-(trimethylsilyl)ethoxy)methyl)-1H-pyrazol-4-yl)-9-(4-methoxybenzyl)-8-phenyl-9H-purine

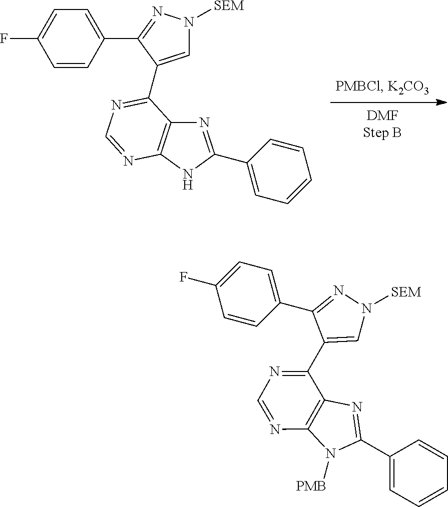

To a stirred mixture of 6-(3-(4-fluorophenyl)-1-((2-(trimethylsilyl)ethoxy)methyl)-1H-pyrazol-4-yl)-8-phenyl-9H-purine (1.26 g, 2.59 mmol, 1 equiv., as a mixture of regioisomers) and K₂CO₃ (0.72 g, 5.18 mmol, 2 equiv) in DMF (12 mL) was added PMBCl (0.49 g, 3.11 mmol, 1.2 equiv) dropwise at RT under nitrogen atmosphere. The resulting mixture was stirred for 4 h at RT under nitrogen atmosphere then quenched by the addition of water (20 mL) at RT. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE:EA (1:1) to afford the title compound (1.41 g, 87%) as a yellow solid (as a mixture of regioisomers). m/z (ESI, +ve ion)=607.20 [M+H]⁺.

### Step 3. 6-(3-(4-Fluorophenyl)-1H-pyrazol-4-yl)-9-(4-methoxybenzyl)-8-phenyl-9H-purine

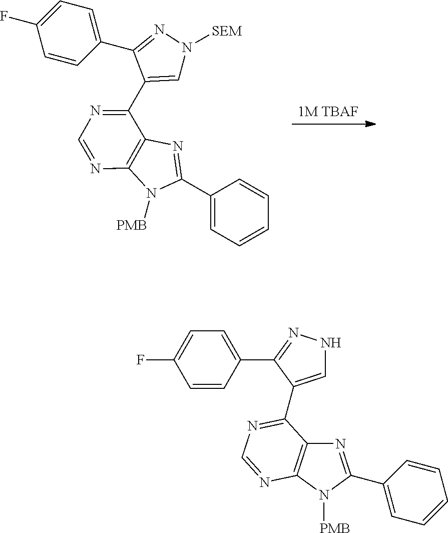

To a flask containing 6-(3-(4-fluorophenyl)-1-((2-(trimethylsilyl)ethoxy)methyl)-1H-pyrazol-4-yl)-9-(4-methoxybenzyl)-8-phenyl-9H-purine (1.41 g, 2.324 mmol, 1 equiv., as a mixture of regioisomers) was added TBAF (20 mL, 1 M in THF) at RT. The resulting mixture was stirred for 16 h at 60° C. under nitrogen atmosphere then concentrated under reduced pressure. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by silica gel column chromatography, eluted with PE:EA (1:1) to afford the title compound (820 mg, 74%) as a yellow solid. m/z (ESI, +ve ion)=477.10 [M+H]⁺.

### Intermediate S. 3-(4-{6-bromofuro[2,3-d]pyrimidin-4-yl}-3-(4-fluorophenyl)pyrazol-1-yl)-1λ6-thietane-1,1-dione

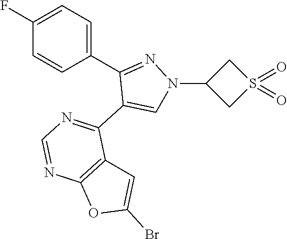 [SMILES: O=S1(=O)CC(n2cc(-c3ncnc4oc(Br)cc34)c(-c3ccc(F)cc3)n2)C1]

### Step 1. 3-(4-{6-bromofuro[2,3-d]pyrimidin-4-yl}-3-(4-fluorophenyl)pyrazol-1-yl)-1λ6-thietane-1,1-dione

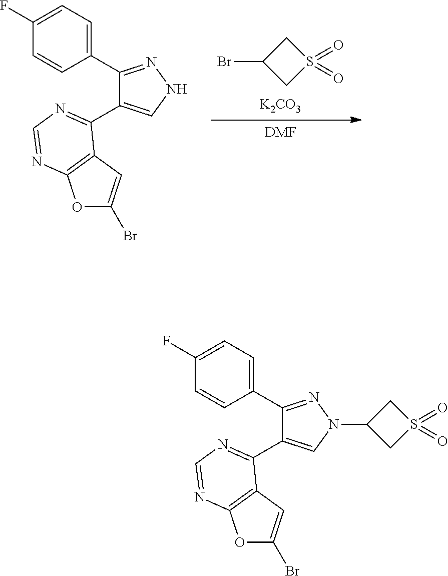

6-Bromo-4-(3-(4-fluorophenyl)-1H-pyrazol-4-yl)furo[2,3-d]pyrimidine (Intermediate Q, 500 mg, 1.39 mmol) and potassium carbonate (393 mg, 2.78 mmol, 2.0 equiv.) were dissolved in DMF (11.2 mL). After stirring for 5 min 3-bromo-1λ6-thietane-1,1-dione (407 mg, 2.09 mmol, 1.5 equiv.) was added. The mixture was further stirred at room temperature for 16 h. H₂O (15 mL), brine and EtOAc (15 mL) were added. The aqueous layer was extracted with additional EtOAc (3×30 mL). The combined organic layers were washed with H₂O (3×20 mL), brine (20 mL), dried over Na₂SO₄, filtered and concentrated. The crude product was purified by column chromatography (EtOAc in hexanes, 10-100%, a gradient elution) to provide the title compound (488 mg, 76%) as a solid. m/z (ESI, +ve ion)=463.0, 465.0 [M+H]⁺.

### Intermediate T. 3-(4-fluorophenyl)-4-{6-iodofuro[2,3-d]pyrimidin-4-yl}-1H-pyrazole

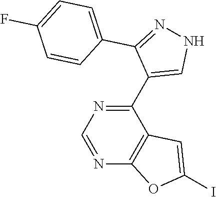 [SMILES: Fc1ccc(-c2n[nH]cc2-c2ncnc3oc(I)cc23)cc1]

### Step 1. 3-(4-fluorophenyl)-4-{6-iodofuro[2,3-d]pyrimidin-4-yl}-1H-pyrazole

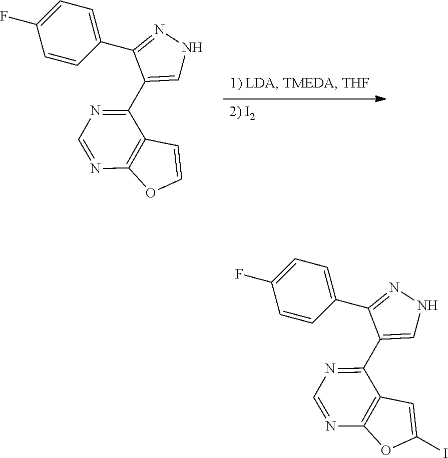

To a solution of 4-(3-(4-Fluorophenyl)-1H-pyrazol-4-yl)furo[2,3-d]pyrimidine (Product of Step B in Intermediate Q, 5 g, 17.840 mmol, 1 equiv.) and N,N,N,N-Tetramethylethylenediamine (4.56 g, 39.248 mmol, 2.2 equiv.) in THF (100 mL, 740.566 mmol) was added dropwise LDA (39 mL, 19.624 mmol, 1.1 equiv., 0.5 M in THF, freshly prepared) at −78° C. under nitrogen atmosphere and stirred for 30 min. Then another batch of LDA (39 mL, 19.624 mmol, 1.1 equiv., 0.5 M in THF, freshly prepared) was added dropwise into the mixture and then stirred for 1 hour at −78° C. A solution of I₂ (5.43 g, 21.408 mmol, 1.2 equiv.) in THF (60 mL, 740.566 mmol) was added dropwise over 5 min at −78° C. The resulting mixture was stirred for additional 1 hour at −78° C. then quenched with sat. NH₄Cl (aq., 50 mL) and sat. Na₂S₂O₃ (aq., 150 mL) at 0° C. The resulting mixture was extracted with EtOAc (3×250 mL). The combined organic layer was washed with brine (2×100 mL), dried over anhydrous Na₂SO₄. After filtration, the filtrate was concentrated under reduced pressure. The residue was purified by silica gel column chromatography, eluted with DCM/MeOH (8/1) to afford the title compound (6 g, 79%) as a yellow solid. m/z (ESI, +ve ion)=406.95 [M+H]⁺. ¹H NMR (400 MHz, DMSO-d₆) δ 13.65-13.59 (m, 1H), 8.68 (s, 1H), 8.54-8.22 (m, 1H), 7.59-7.55 (m, 2H), 7.28-7.20 (m, 3H).

### Intermediate U. 3-[3-(4-fluorophenyl)-4-{6-iodofuro[2,3-d]pyrimidin-4-yl}pyrazol-1-yl]-16-thietane-1,1-dione

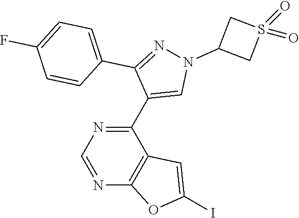 [SMILES: O=S1(=O)CC(n2cc(-c3ncnc4oc(I)cc34)c(-c3ccc(F)cc3)n2)C1]

### Step 1. 3-[3-(4-fluorophenyl)-4-{6-iodofuro[2,3-d]pyrimidin-4-yl}pyrazol-1-yl]-1λ6-thietane-1,1-dione

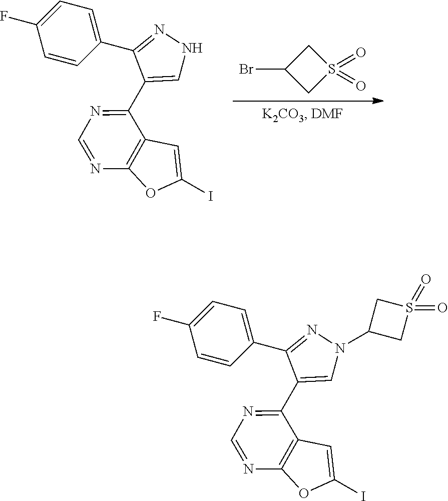

To a stirred solution of 3-(4-fluorophenyl)-4-{6-iodofuro[2,3-d]pyrimidin-4-yl}-1H-pyrazole (Intermediate T, 12.5 g, 30.776 mmol, 1 equiv.) and K₂CO₃ (8.51 g, 61.552 mmol, 2 equiv.) in DMF (70 m) was added 3-bromo-1,6-thietane-1,1-dione (6.83 g, 36.931 mmol, 1.2 equiv.) at room temperature under nitrogen atmosphere. After the resulting mixture was stirred overnight at room temperature under nitrogen, it was quenched by the addition of water (180 mL). The resulting mixture was extracted with EtOAc (3×200 mL). The combined organic layer was washed with brine (3×200 mL), dried over anhydrous Na₂SO₄. After filtration, the filtrate was concentrated under reduced pressure. The residue was purified by trituration with EtOAc (150 mL). The precipitated solids were collected by filtration and washed with EtOAc (3×20 mL). This resulted in the title compound (14 g, 89%) as a white solid. m/z (ESI, +ve ion)=510.95

### Intermediate V. 3-(3-(4-Fluorophenyl)-4-(6-(1,2,3,6-tetrahydropyridin-4-yl)furo[2,3-d]pyrimidin-4-yl)-1H-pyrazol-1-yl)thietane 1,1-dioxide

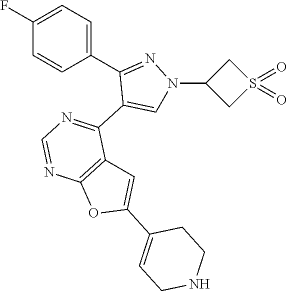 [SMILES: O=S1(=O)CC(n2cc(-c3ncnc4oc(C5=CCNCC5)cc34)c(-c3ccc(F)cc3)n2)C1]

### Step 1. tert-Butyl 4-(4-(1-(1,1-dioxidothietan-3-yl)-3-(4-fluorophenyl)-1H-pyrazol-4-yl)furo[2,3-d]pyrimidin-6-yl)-5,6-dihydropyridine-1 (2H)-carboxylate

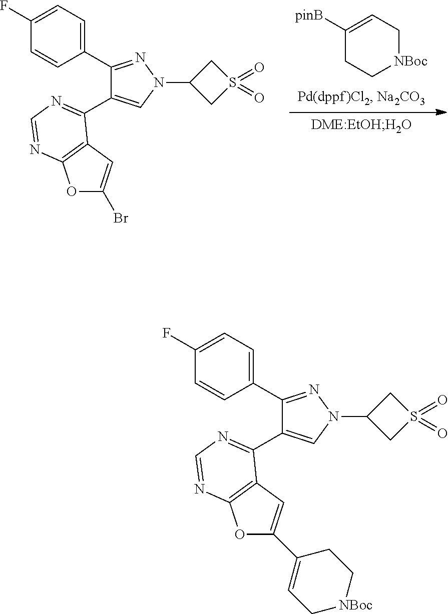

3-(4-{6-bromofuro[2,3-d]pyrimidin-4-yl}-3-(4-fluorophenyl)pyrazol-1-yl)-1,6-thietane-1,1-dione (Intermediate S, 400 mg, 863 μmol), tert-butyl 4-(4,4,5,5-tetramethyl-1,3,2-dioxaborolan-2-yl)-5,6-dihydropyridine-1 (2H)-carboxylate (327 mg, 1.04 mmol, 1.2 equiv.), sodium carbonate (277 mg, 2.59 mmol, 3.0 equiv.) and 1,1′-bis(diphenylphosphino)ferrocene dichloropalladium (II) (66.5 mg, 86.3 μmol, 0.1 equiv.) were dissolved in a mixture of DME (5.60 mL), EtOH (2.80 mL), and H₂O (1.40 mL). The mixture was purged with nitrogen then heat to 85° C. for 16 hours. The reaction mixture was cooled to room temperature and diluted with water, extracted with EtOAc (×3), washed with brine, dried over Na₂SO₄, filtered, and concentrated under reduced pressure. The crude material was purified by column chromatography (50-100% EtOAc in heptanes, a gradient elution) to provide the title compound (413 mg, 66%) as a solid. m/z (ESI, +ve ion)=566.4 [M+H]⁺.

### Step 2. 3-(3-(4-Fluorophenyl)-4-(6-(1,2,3,6-tetrahydropyridin-4-yl)furo[2,3-d]pyrimidin-4-yl)-1H-pyrazol-1-yl)thietane 1,1-dioxide

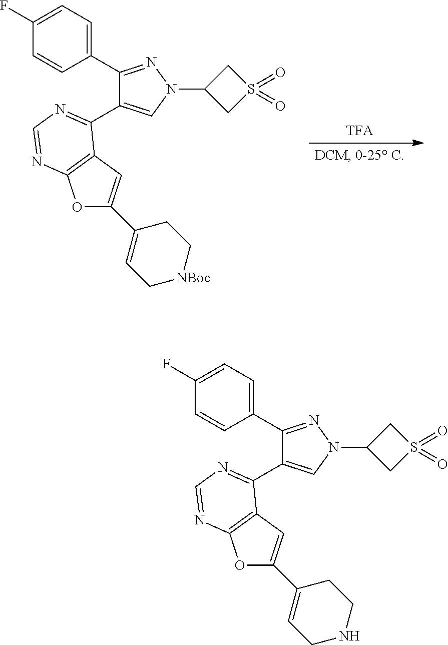 [SMILES: CC(C)(C)OC(=O)N1CC=C(c2cc3c(-c4cn(C5CS(=O)(=O)C5)nc4-c4ccc(F)cc4)ncnc3o2)CC1 ; "DCM, 0-25° C." ; O=S1(=O)CC(n2cc(-c3ncnc4oc(C5=CCNCC5)cc34)c(-c3ccc(F)cc3)n2)C1 ; "TFA"]

Tert-butyl 4-(4-(1-(1,1-dioxidothietan-3-yl)-3-(4-fluorophenyl)-1H-pyrazol-4-yl)furo[2,3-d]pyrimidin-6-yl)-5,6-dihydropyridine-1 (2H)-carboxylate (413 mg, 730 μmol) was dissolved in DCM (1.83 mL) and the mixture was cooled to 0° C. Trifluoroacetic acid (565 μL, 7.30 mmol, 10.0 equiv.) was added and the mixture was stirred at room temperature for 1 hour. The solvent was evaporated and the pH was adjusted to 7-8 using 15% w/w aqueous NaOH solution. The aqueous layer was extracted with EtOAc (×3) and the combined organic layers were washed with brine, dried over Na₂SO₄, filtered, and evaporated to afford the title compound (340 mg, quant) as a solid. m/z (ESI, +ve ion)=466.3 [M+H]⁺.

### Intermediate W. 3-[3-(4-fluorophenyl)-4-[6-(4,4,5,5-tetramethyl-1,3,2-dioxaborolan-2-yl)furo[2,3-d]pyrimidin-4-yl]pyrazol-1-yl]-1λ6-thietane-1,1-dione

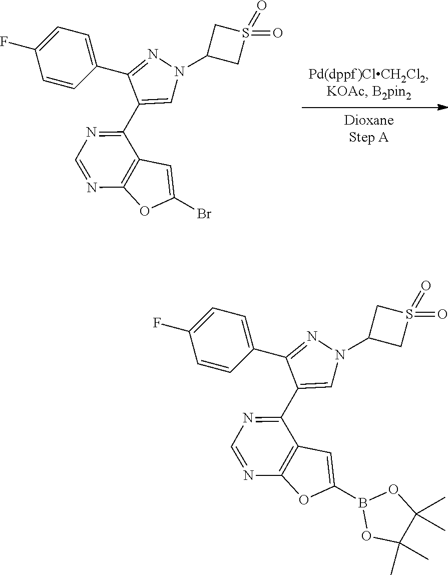 [SMILES: O=S1(=O)CC(n2cc(-c3ncnc4oc(Br)cc34)c(-c3ccc(F)cc3)n2)C1 ; "DioxaneStep A" ; "Pd(dppf)Cl·CH2Cl2,KOAc, B2pin2" ; CC1(C)OB(c2cc3c(-c4cn(C5CS(=O)(=O)C5)nc4-c4ccc(F)cc4)ncnc3o2)OC1(C)C]

To a stirred solution of 3-(4-{6-bromofuro[2,3-d]pyrimidin-4-yl}-3-(4-fluorophenyl)pyrazol-1-yl)-1λ6-thietane-1,1-dione (Intermediate S, 600 mg, 1.295 mmol, 1 equiv.) and bis(pinacolato)diboron (427.55 mg, 1.683 mmol, 1.3 equiv.) in 1,4-dioxane (6 mL) were added Pd(dppf)Cl₂·CH₂Cl₂ (211.01 mg, 0.259 mmol, 0.2 equiv.) and KOAc (381.31 mg, 3.885 mmol, 3 equiv.) at room temperature. The resulting mixture was stirred for 3 h at 100° C. under nitrogen then cooled to room temperature. The resulting mixture was filtered and the filter cake was washed with EtOAc (5×5 mL). The filtrate was concentrated under reduced pressure to give the title compound (925 mg, 56%) as a brown oil. The crude product was used in the next step directly without further purification. m/z (ESI, +ve ion)=511.20 [M+H]⁺.

### Intermediate X. 1-[3-(4-fluorophenyl)-4-{6-iodofuro[2,3-d]pyrimidin-4-yl}pyrazol-1-yl]-2-methylpropan-2-ol

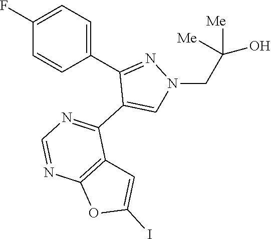 [SMILES: CC(C)(O)Cn1cc(-c2ncnc3oc(I)cc23)c(-c2ccc(F)cc2)n1]

### Step 1. 1-[3-(4-fluorophenyl)-4-{6-iodofuro[2,3-d]pyrimidin-4-yl}pyrazol-1-yl]-2-methylpropan-2-ol

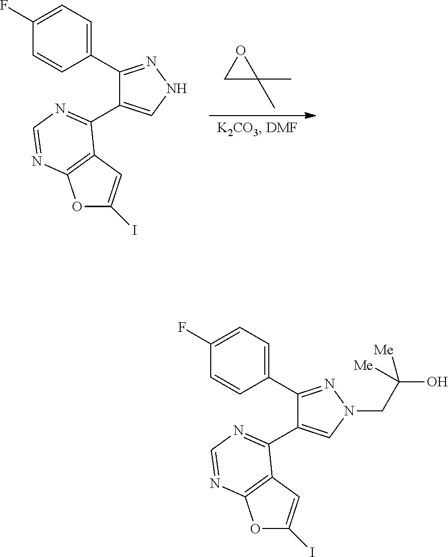

To a stirred solution of 3-(4-fluorophenyl)-4-{6-iodofuro[2,3-d]pyrimidin-4-yl}-1H-pyrazole (Intermediate T, 400 mg, 0.980 mmol, 1 equiv.) and 2,2-dimethyloxirane (142.0 mg, 1.960 mmol, 2 equiv.) in DMF (0.5 mL) were added K₂CO₃ (272.2 mg, 1.960 mmol, 2 equiv.) at room temperature in a sealed tube. After the resulting mixture was stirred for 16 h at 50° C., it was cooled to room temperature. The reaction was quenched by the addition of water (30 mL) at room temperature and extracted with EtOAc (3×20 mL). The combined organic layer was washed with brine (3×10 mL), dried over anhydrous Na₂SO₄. After filtration, the filtrate was concentrated under reduced pressure. The residue was purified by Prep-TLC (PE/EtOAc=1/3) to afford the title compound (220 mg, 46%) as a white solid. m/z (ESI, +ve ion)=479.05

### Intermediate Y. 3-(4-fluorophenyl)-4-{6-iodofuro[2,3-d]pyrimidin-4-yl}-1-[(cis)-3-methanesulfonylcyclobutyl]pyrazole

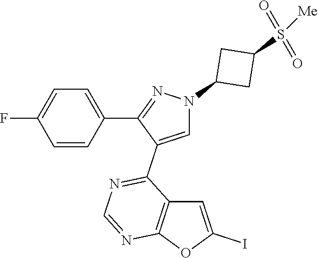 [SMILES: CS(=O)(=O)[C@H]1C[C@@H](n2cc(-c3ncnc4oc(I)cc34)c(-c3ccc(F)cc3)n2)C1]

### Step 1. (trans)-3-methanesulfonylcyclobutyl 4-methylbenzenesulfonate

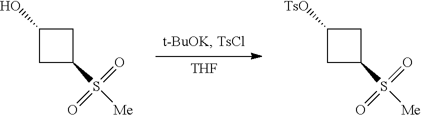

To a stirred mixture of (trans)-3-methanesulfonylcyclobutan-1-ol (3 g, 19.975 mmol, 1 equiv.) in THF (30 mL) were added potassium tert-butoxide (4.48 g, 39.950 mmol, 2 equiv.) in portions at room temperature under nitrogen. The resulting mixture was stirred for 0.5 h at room temperature under nitrogen. To the above mixture was added TsCl (4.57 g, 23.970 mmol, 1.2 equiv.) in portions at room temperature. The resulting mixture was stirred for additional 3 h at room temperature then quenched by the addition of sat. NH₄Cl (aq., 10 mL). The resulting mixture was extracted with EtOAc (3×40 mL). The combined organic layer was washed with brine (2×30 mL), dried over anhydrous Na₂SO₄. After filtration, the filtrate was concentrated under reduced pressure. The residue was purified by silica gel column chromatography, eluted with PE/EtOAc (1/1) to afford the title compound (5.38 g, 88%) as a white solid. m/z (ESI, +ve ion)=326.95 [M+Na]⁺.

### Step 2. 3-(4-fluorophenyl)-4-{6-iodofuro[2,3-d]pyrimidin-4-yl}-1-[(cis)-3-methanesulfonylcyclobutyl]pyrazole

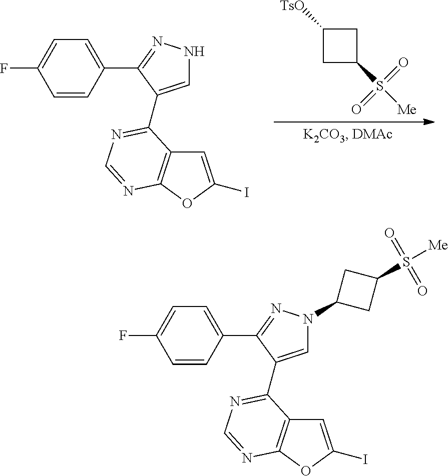

To a stirred solution of 3-(4-fluorophenyl)-4-{6-iodofuro[2,3-d]pyrimidin-4-yl}-1H-pyrazole (Intermediate T, 1 g, 2.462 mmol, 1 equiv) and (trans)-3-methanesulfonylcyclobutyl 4-methylbenzenesulfonate (0.90 g, 2.954 mmol, 1.2 equiv.) in DMAc (10 mL) was added K₂CO₃ (0.51 g, 3.693 mmol, 1.5 equiv.) at room temperature under nitrogen. The resulting mixture was stirred for 16 h at 70° C. under nitrogen then quenched with sat. NH₄Cl (aq., 30 mL). The resulting mixture was extracted with EtOAc (3×20 mL). The combined organic layer was washed with brine (2×35 mL), dried over anhydrous Na₂SO₄. After filtration, the filtrate was concentrated under reduced pressure. The residue was purified by silica gel column chromatography, eluted with 50-100% EtOAc (plus 1% TEA) in PE (plus 1% TEA) to afford the title compound (250 mg, 19%) as a yellow solid. m/z (ESI, +ve ion)=539.00 [M+H]⁺.

### Intermediate Z. 4-(1-(3,3-difluoropropyl)-3-(4-fluorophenyl)-1H-pyrazol-4-yl)-6-iodofuro[2,3-d]pyrimidine

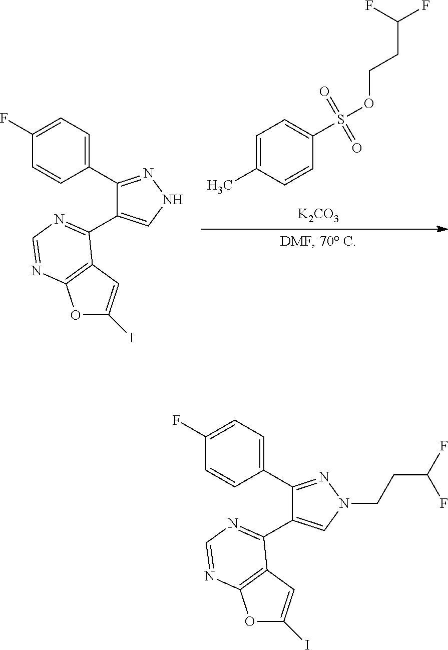

3-Fluoropropyl 4-methylbenzenesulfonate (924 mg, 3.69 mmol, 1.5 equiv.) was added to a mixture of 3-(4-fluorophenyl)-4-{6-iodofuro[2,3-d]pyrimidin-4-yl}-1H-pyrazole (Intermediate T, 1.00 g, 2.46 mmol, 1.0 equiv.) and potassium carbonate (694 mg, 4.92 mmol, 2.0 equiv.) in DMF (24.6 mL). The mixture was stirred for 15 h at 70° C. then diluted with saturated aqueous NH₄Cl (75 mL) and extracted with EtOAc (3×50 mL). The combined organic layers were washed with aqueous 1M NaOH, brine, then dried (Na₂SO₄), filtered and concentrated under reduced pressure to provide the title compound (1.40 g, quantitative) as an oil. The material was used in the next steps without further purification. Contains 25% of N² regioisomer. m/z (ESI, +ve ion)=485.1 [M+H]⁺.

### Intermediate AA. 4-(1-((1s,3s)-3-fluorocyclobutyl)-3-(4-fluorophenyl)-1H-pyrazol-4-yl)-6-iodofuro[2,3-d]pyrimidine

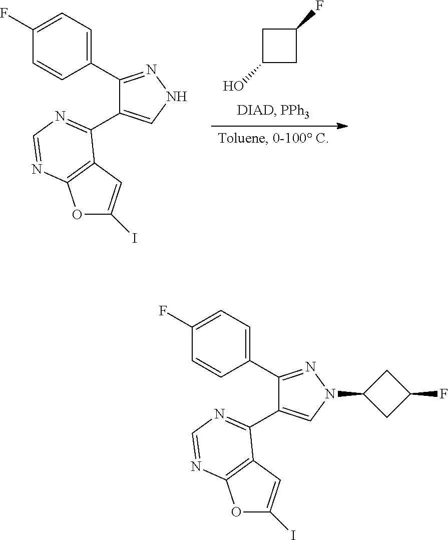

To a degassed solution of triphenylphosphine (1.37 g, 5.17 mmol, 2.1 equiv.) and diisopropyl-azodicarboxylate (989 μL, 4.92 mmol, 2.0 equiv.) in toluene (20.0 mL) at 0° C. was added trans-3-fluorocyclobutanol (266 mg, 2.95 mmol, 1.2 equiv.) and 3-(4-fluorophenyl)-4-{6-iodofuro[2,3-d]pyrimidin-4-yl}-1H-pyrazole (Intermediate T, 1.00 g, 2.46 mmol, 1.0 equiv.). The mixture was stirred at 70° C. for 16 h then cooled to room temperature and diluted with water (25 mL). The aqueous layer was extracted with EtOAc (3×50 mL). The combined organic layers were washed with brine then dried (Na₂SO₄), filtered and concentrated under reduced pressure. The crude was purified by column chromatography (EtOAc in hexanes, 0-60%, a gradient elution) to provide the title compound (526 mg, 45%). ¹H NMR (400 MHz, DMSO-d₆) δ 8.67 (s, 1H), 8.65 (s, 1H), 7.61-7.51 (m, 2H), 7.35 (s, 1H), 7.19 (t, J=8.9 Hz, 2H), 5.06 (dp, J=56.5, 6.7 Hz, 1H), 4.67-4.51 (m, 1H), 3.05-2.76 (m, 4H). m/z (ESI, +ve ion)=479.1 [M+H]⁺.

### Intermediate AB. 3-(3-(4-fluorophenyl)-4-(6-(1-methyl-1H-imidazol-4-yl)furo[2,3-d]pyrimidin-4-yl)-1H-pyrazol-1-yl)propanenitrile

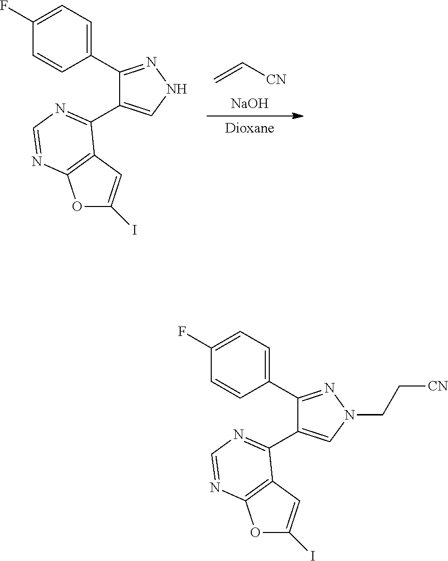

Acrylonitrile (273 μL, 4.11 mmol, 2.0 equiv.) was added to a mixture of 3-(4-fluorophenyl)-4-{6-iodofuro[2,3-d]pyrimidin-4-yl}-1H-pyrazole (Intermediate T, 835 mg, 2.06 mmol, 1.0 equiv.) and 50% sodium hydroxide (1.1 mL, 220 mmol, 106 equiv.) in dioxane (2.06 mL). The mixture was stirred for 0.5 h at 80° C. then cooled to room temperature and diluted with water (15 mL). The aqueous layer was extracted with Et₂O (3×10 mL) and the combined organic layers were washed with water, dried (MgSO₄), filtered and concentrated under reduced pressure. The residue was precipitated in EtOAc and the precipitate was filtered and washed with EtOAc to provide the title compound (627 mg, 66%) as a solid. ¹H NMR (400 MHz, DMSO-d₆) δ 8.70 (s, 1H), 8.68 (s, 1H), 7.60-7.56 (m, 2H), 7.23 (s, 1H), 7.22-7.17 (m, 2H), 4.54 (t, J=6.4 Hz, 2H), 3.22 (t, J=6.4 Hz, 2H). m/z (ESI, +ve ion)=460.1 [M+H]⁺.

### Intermediate AC. 4-(1-(3-((di-tert-butyl(phenyl)silyl)oxy)-2,2-difluoropropyl)-3-(4-fluorophenyl)-1H-pyrazol-4-yl)-6-iodofuro[2,3-d]pyrimidine

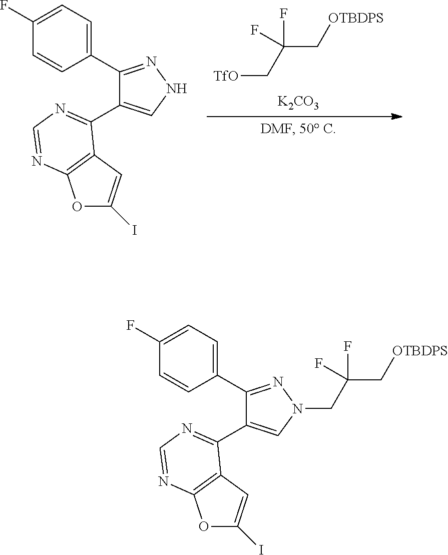

Potassium carbonate (694 mg, 4.92 mmol, 2.0 equiv) was added to a mixture of 3-(4-fluorophenyl)-4-{6-iodofuro[2,3-d]pyrimidin-4-yl}-1H-pyrazole (Intermediate T, 1.00 g, 2.46 mmol, 1.0 equiv.) and 3-((tert-butyldiphenylsilyl)oxy)-2,2-difluoropropyl trifluoromethanesulfonate (1.16 mL, 2.95 mmol, 1.2 equiv.) in DMF (24.6 mL). The mixture was stirred for 5 h at 50° C. then cooled to room temperature. The mixture was diluted with water (200 mL) and extracted with EtOAc (3×100 mL). The combined organic layers were washed with water, brine, then dried (Na₂SO₄), filtered and concentrated under reduced pressure to provide the title compound (1.80 g, 99%) as a crude solid. The material was used in the next step without further purification. ¹H NMR (400 MHz, CDCl₃) δ 8.83 (s, 1H), 8.09 (s, 1H), 7.65 (d, J=6.9 Hz, 5H), 7.40 (d, J=7.4 Hz, 3H), 7.31 (t, J=7.4 Hz, 4H), 7.05 (t, J=8.6 Hz, 2H), 6.23 (s, 1H), 4.78 (t, J=12.8 Hz, 2H), 3.82 (t, J=11.8 Hz, 2H), 1.10 (s, 9H). Contains 10% of N² regioisomer. m/z (ESI, +ve ion)=739.0 [M+H]⁺.

### Intermediate AD. (cis)-3-cyanocyclobutyl 4-methylbenzenesulfonate

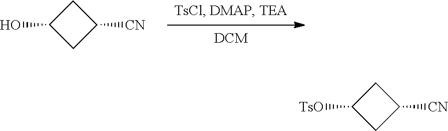

To a stirred solution of (trans)-3-hydroxycyclobutane-1-carbonitrile (97.1 mg, 1.000 mmol, 1 equiv) and TsCl (228.73 mg, 1.200 mmol, 1.2 equiv) in DCM (3 mL) were added TEA (202.35 mg, 2.000 mmol, 2 equiv) and DMAP (12.21 mg, 0.100 mmol, 0.1 equiv) at room temperature under nitrogen atmosphere. The resulting mixture was stirred for 3 h at room temperature under nitrogen atmosphere and concentrated under reduced pressure. The residue was purified by prep-TLC (PE:EtOAc=1:1) to afford the title compound (200 mg, 80%) as a white solid. ¹H NMR (300 MHz, Chloroform-d) δ 7.82-7.72 (m, 2H), 7.41-7.30 (m, 2H), 4.83-4.67 (m, 1H), 2.78-2.58 (m, 3H), 2.59-2.49 (m, 2H), 2.46 (s, 3H).

### Intermediate AE. 3-(4-fluorophenyl)-4-[6-(1-methylimidazol-4-yl)furo[2,3-d]pyrimidin-4-yl]-1H-pyrazole

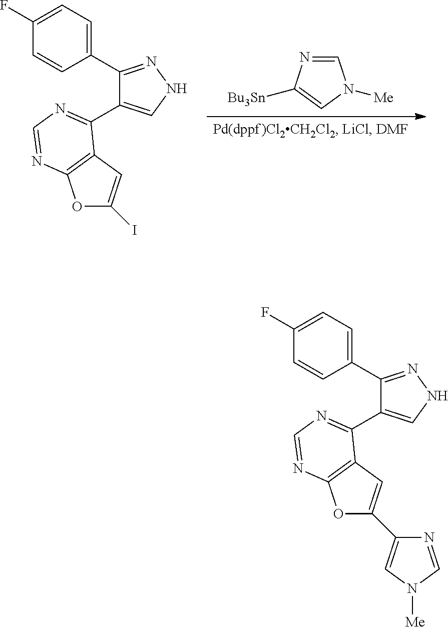

To a stirred solution of 3-(4-fluorophenyl)-4-{6-iodofuro[2,3-d]pyrimidin-4-yl}-1H-pyrazole (Intermediate T, 4 g, 9.848 mmol, 1 equiv) and 1-methyl-4-(tributylstannyl)imidazole (Product of Step 1 en route to Example 200, 9.14 g, 24.6 mmol, 2.5 equiv) in DMF (100 mL) was added Pd(dppf)Cl₂·CH₂Cl₂ (0.80 g, 0.99 mmol, 0.1 equiv) and LiCl (1.25 g, 29.5 mmol, 3 equiv) at room temperature under nitrogen atmosphere. The resulting mixture was stirred for 36 h at 80° C. under nitrogen atmosphere. The mixture was allowed to cool down to room temperature. The resulting mixture was diluted with water (100 mL). The mixture was extracted with EtOAc (3×100 mL). The combined organic layers were washed with brine (2×100 mL) and dried over anhydrous Na₂SO₄. After filtration, the filtrate was concentrated under reduced pressure. The residue was purified by silica gel column chromatography and eluted with DCM:MeOH (10:1) to afford the title compound (2.1 g, 59%) as a grey solid. m/z (ESI, +ve ion)=361.10 [M+H]⁺.

### Example 2. 3-[1-(4-Fluorophenyl)-5-(2-phenyl-1H-pyrrolo[2,3-b]pyridin-4-yl)-1H-1,2,4-triazol-3-yl]propan-1-ol

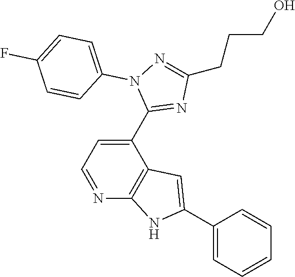 [SMILES: OCCCc1nc(-c2ccnc3[nH]c(-c4ccccc4)cc23)n(-c2ccc(F)cc2)n1]

### Step 1. 2-Phenyl-1H-pyrrolo[2,3-b]pyridine-4-carbonitrile

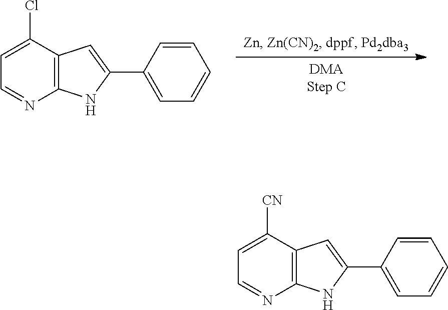 [SMILES: Clc1ccnc2[nH]c(-c3ccccc3)cc12 ; "DMAStep C" ; "Zn, Zn(CN)2, dppf, Pd2dba3" ; N#Cc1ccnc2[nH]c(-c3ccccc3)cc12]

To a stirred mixture of 4-chloro-2-phenyl-1H-pyrrolo[2,3-b]pyridine (Intermediate A, 500 mg, 2.19 mmol, 1.0 equiv), Zn (14.3 mg, 0.219 mmol, 0.1 equiv) and Zn(CN)₂ (154 mg, 1.312 mmol, 0.6 equiv) in DMA (5 mL) were added 1,1-ferrocenediyl-bis(diphenylphosphine) (dppf) (36.2 mg, 0.066 mmol, 0.03 equiv) and Pd₂(dba)₃ (30 mg, 0.033 mmol, 0.015 equiv) at RT under nitrogen atmosphere. The resulting mixture was stirred for 4 h at 120° C. under nitrogen atmosphere. The resulting mixture was diluted with water (10 mL). The precipitated solids were collected by filtration and washed with water (3×30 mL) to afford the title compound (400 mg, 83%) as an off-white solid. m/z (ESI, +ve ion)=218.10 [M−H]⁻.

### Step 2. 2-Phenyl-1H-pyrrolo[2,3-b]pyridine-4-carboxylic acid

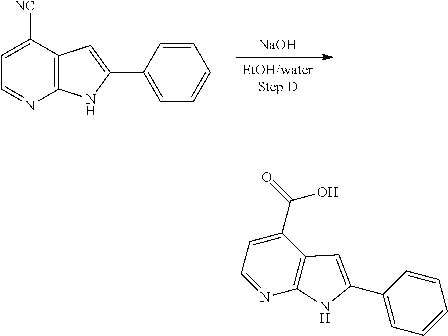 [SMILES: N#Cc1ccnc2[nH]c(-c3ccccc3)cc12 ; "NaOH" ; "EtOH/waterStep D" ; O=C(O)c1ccnc2[nH]c(-c3ccccc3)cc12]

To a stirred mixture of 2-phenyl-1H-pyrrolo[2,3-b]pyridine-4-carbonitrile (500 mg, 2.28 mmol, 1.00 equiv) in EtOH (5 mL) and H₂O (5 mL) was added NaOH (913 mg, 22.8 mmol, 10 equiv) in portions at RT. The resulting mixture was stirred for overnight at 85° C. and then cool down to RT. The mixture was acidified to pH 2 with conc. HCl (10 mL). The resulting mixture was diluted with water (10 mL). The precipitated solid was collected by filtration and washed with water (3×20 mL) to afford the title compound (400 mg, 74%) as a yellow solid. m/z (ESI, +ve ion)=237.10 [M−H]⁻.

### Step 3. Methyl 3-[1-(4-fluorophenyl)-5-{2-phenyl-1H-pyrrolo[2,3-b]pyridin-4-yl}-1,2,4-triazol-3-yl]propanoate

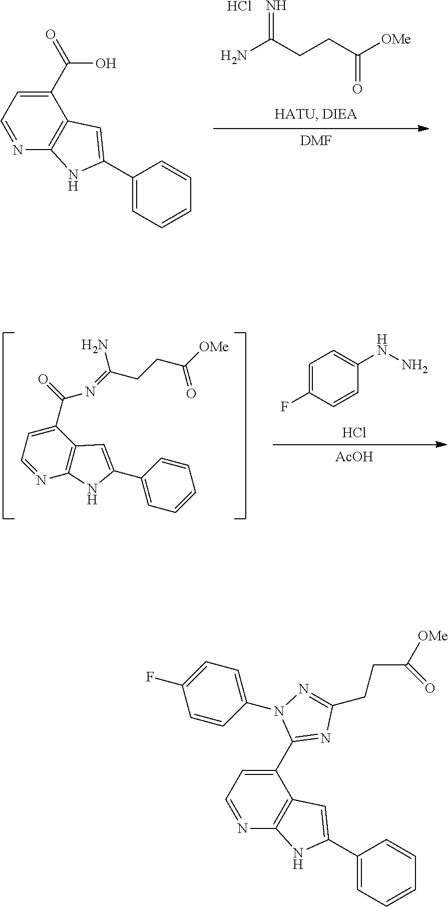

To a stirred mixture of 2-phenyl-1H-pyrrolo[2,3-b]pyridine-4-carboxylic acid (240 mg, 1.01 mmol, 1.00 equiv) and methyl 3-carbamimidoylpropanoate hydrochloride (336 mg, 2.01 mmol, 2 equiv) in DMF (4 mL) were added HATU (460 mg, 1.21 mmol, 1.2 equiv) and DIEA (391 mg, 3.02 mmol, 3 equiv) at RT under nitrogen atmosphere. The resulting mixture was stirred for 16 h at RT under nitrogen atmosphere. To the above mixture were added (4-fluorophenyl)hydrazine hydrochloride (246 mg, 1.51 mmol, 1.5 equiv) and AcOH (605 mg, 10.1 mmol, 10 equiv) at RT. The resulting mixture was stirred for additional 3 h at 80° C. and then concentrated under reduced pressure. The residue was purified by reverse flash chromatography, eluted with 50% ACN in water (0.1% TFA) to afford the title compound (300 mg, 68%) as an off-white solid. m/z (ESI, +ve ion)=442.10 [M+H]⁺.

### Step 4. 3-[1-(4-Fluorophenyl)-5-(2-phenyl-1H-pyrrolo[2,3-b]pyridin-4-yl)-1H-1,2,4-triazol-3-yl]propan-1-ol

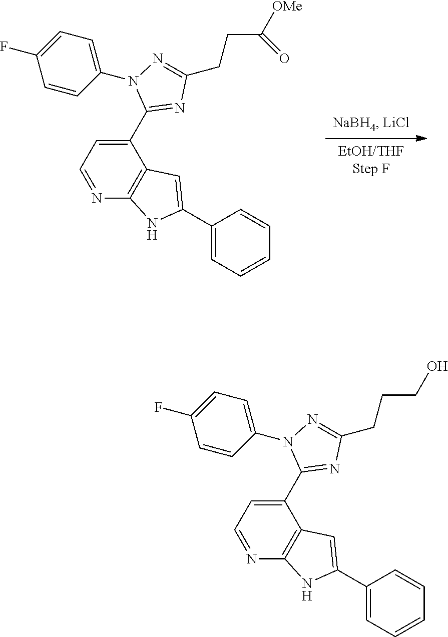

To a stirred mixture of methyl 3-[1-(4-fluorophenyl)-5-{2-phenyl-1H-pyrrolo[2,3-b]pyridin-4-yl}-1,2,4-triazol-3-yl]propanoate (180 mg, 0.41 mmol, 1.00 equiv) in THF (2 mL) and EtOH (2 mL) were added LiCl (3.46 mg, 0.082 mmol, 0.2 equiv) and NaBH₄ (123 mg, 3.26 mmol, 8 equiv) at 0° C. under nitrogen atmosphere. The resulting mixture was stirred for 16 h at RT under nitrogen atmosphere. The reaction was quenched by the addition of water (10 mL) at 0° C. The resulting mixture was concentrated under reduced pressure. The residue was purified by prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 32% B to 42% B in 8 min, 42% B; Wavelength: 254 nm; RT1 (min): 7.13 to afford Example 2 (70 mg, 41%) as an off-white solid. m/z (ESI, +ve ion)=414.20 [M+H]⁺. ¹H-NMR (400 MHz, DMSO-d₆) δ 12.74 (s. 1H), 8.21 (d, J=5.2 Hz, 1H), 7.88 (d, J=7.6 Hz, 2H), 7.48 (t, J=7.2 Hz, 4H), 7.39-7.36 (m, 1H), 7.32-7.28 (m, 2H), 6.93 (d, J=5.2 Hz, 1H), 6.83 (s, 1H), 4.57 (t, J=5.2 Hz, 1H), 3.60-3.56 (m, 2H), 2.86 (t, J=7.6 Hz, 2H), 2.01-1.94 (m, 2H).

### Example 3. 3-[5-(4-Fluorophenyl)-1-(2-phenyl-1H-pyrrolo[2,3-b]pyridin-4-yl)-1H-1,2,4-triazol-3-yl]propan-1-ol

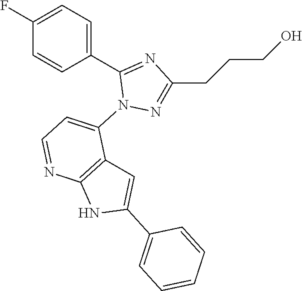 [SMILES: OCCCc1nc(-c2ccc(F)cc2)n(-c2ccnc3[nH]c(-c4ccccc4)cc23)n1]

### Step 1. Ethyl 4-[(tert-butyldiphenylsilyl)oxy]butanoate

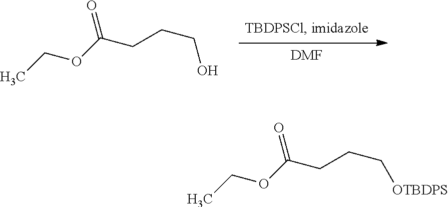

To a stirred mixture of ethyl 4-hydroxybutanoate (1 g, 7.57 mmol, 1.00 equiv) and imidazole (1.24 g, 18.2 mmol, 2.4 equiv) in DMF (10 mL) was added tert-butyl(chloro)diphenylsilane (2.50 g, 9.08 mmol, 1.2 equiv) dropwise over 10 min at 0° C. under nitrogen atmosphere. The resulting mixture was stirred for 2 h at RT under nitrogen atmosphere. The reaction was quenched by the addition of water (300 mL) at RT and extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with PE/EtOAc (1/1) to afford the title compound (2.5 g, 89%) as a colorless oil. m/z (ESI, +ve ion)=371.20 [M+H]⁺.

### Step 2. 4-[(tert-Butyldiphenylsilyl)oxy]butanoic acid

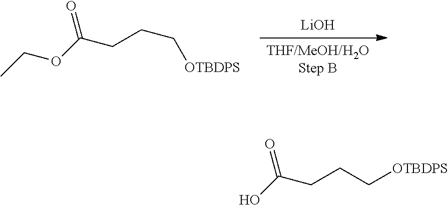

To a stirred mixture of ethyl 4-[(tert-butyldiphenylsilyl)oxy]butanoate (500 mg, 1.35 mmol, 1.00 equiv) in THF (4.5 mL), H₂O (4.5 mL) and MeOH (4.5 mL) was added LiOH (64.6 mg, 2.70 mmol, 2 equiv) in portions at RT. The resulting mixture was stirred for 2 h at RT then quenched by the addition of water (200 mL) at RT. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with DCM:MeOH (20:1) to afford the title compound (410 mg, 89%) as a colorless oil. m/z (ESI, +ve ion)=343.25 [M+H]⁺.

### Step 3. Ethyl 4-fluorobenzenecarboximidate

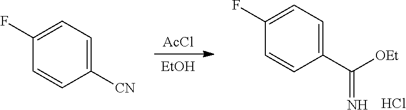 [SMILES: "EtOH" ; "AcCl" ; N#Cc1ccc(F)cc1 ; CCOC(=N)c1ccc(F)cc1 ; Cl]

A mixture of 4-fluorobenzonitrile (5 g, 41.3 mmol, 1.00 equiv) in EtOH (23 g, 495 mmol, 12 equiv) and AcCl (25.9 g, 330 mmol, 8 equiv) was stirred for overnight at RT under nitrogen atmosphere. The resulting mixture was diluted with water (300 mL) and extracted with EtOAc (3×200 mL). The combined organic layers were washed with brine (3×100 mL), dried over anhydrous Na₂SO₄. After filtration, the filtrate was concentrated under reduced pressure to afford the title compound (6.5 g, 77%) as a white solid. m/z (ESI, +ve ion)=168.25 [M+H]⁺.

### Step 4. N-Amino-4-fluorobenzenecarboximidamide

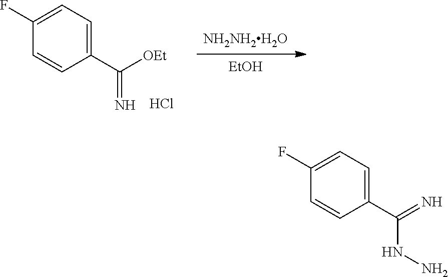 [SMILES: "EtOH" ; CCOC(=N)c1ccc(F)cc1 ; Cl ; "NH2NH2·H2O" ; N=C(NN)c1ccc(F)cc1]

To a stirred mixture of ethyl 4-fluorobenzenecarboximidate (1 g, 4.91 mmol, 1.00 equiv) in EtOH (20 mL) was added NH₂NH₂·H₂O (270 mg, 5.40 mmol, 1.1 equiv) dropwise over 5 min at 0° C. The resulting mixture was stirred for 2 h at 0° C. then warmed up to RT. The resulting mixture was concentrated under reduced pressure. The residue was purified by trituration with DCM (60 mL) to afford the title compound (500 mg, 67%) as an off-white solid. m/z (ESI+ve ion)=154.05 [M+H]⁺.

### Step 5. 3-{3-[(tert-Butyldiphenylsilyl)oxy]propyl}-5-(4-fluorophenyl)-1H-1,2,4-triazole

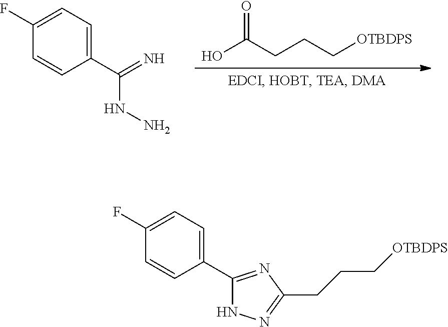

To a stirred mixture of N-amino-4-fluorobenzenecarboximidamide (450 mg, 2.938 mmol, 1.00 equiv), 4-[(tert-butyldiphenylsilyl)oxy]butanoic acid (1.21 g, 3.53 mmol, 1.2 equiv), EDC (547 mg, 3.53 mmol, 1.2 equiv) and HOBT (516 mg, 3.82 mmol, 1.3 equiv) in DMA (4.5 mL) was added TEA (892 mg, 8.81 mmol, 3 equiv) at RT under nitrogen atmosphere. The resulting mixture was stirred for overnight at 110° C. under nitrogen atmosphere then cooled down to RT. The reaction was quenched by the addition of water (150 mL) at RT and extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with DCM:MeOH (20:1) to afford the title compound (180 mg, 13%) as an orange oil. m/z (ESI, +ve ion)=460.40 [M+H]⁺.

### Step 6. 3-{3-[(tert-Butyldiphenylsilyl)oxy]propyl}-5-(4-fluorophenyl)-1-[1-(methoxymethyl)-2-phenylpyrrolo[2,3-b]pyridin-4-yl]-1,2,4-triazole

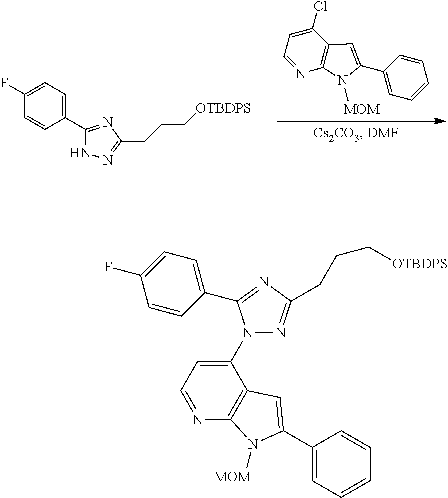

To a stirred mixture of 3-{3-[(tert-butyldiphenylsilyl)oxy]propyl}-5-(4-fluorophenyl)-1H-1,2,4-triazole (180 mg, 0.392 mmol, 1.00 equiv) and 4-chloro-1-(methoxymethyl)-2-phenylpyrrolo[2,3-b]pyridine (Intermediate B, 128 mg, 0.47 mmol, 1.2 equiv) in DMF (2 mL) was added Cs₂CO₃ (256 mg, 0.78 mmol, 2 equiv) at RT under nitrogen atmosphere. The resulting mixture was stirred for 2 h at 60° C. under nitrogen atmosphere then cooled down to RT. The reaction was quenched by the addition of water (50 mL) and extracted with EtOAc (3×30 mL). The combined organic layers were washed with brine (2×30 mL), dried over anhydrous Na₂SO₄. After filtration, the filtrate was concentrated under reduced pressure. The residue was purified by reverse flash chromatography with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 78% B to 85% B in 8 min, 85% B; Wavelength: 254 nm; RT1 (min): 8 to afford the title compound (62 mg, 23%) as a yellow solid. m/z (ESI+ve ion)=696.30 [M+H]⁺.

### Step 7. 3-[5-(4-Fluorophenyl)-1-(2-phenyl-1H-pyrrolo[2,3-b]pyridin-4-yl)-1H-1,2,4-triazol-3-yl]propan-1-ol

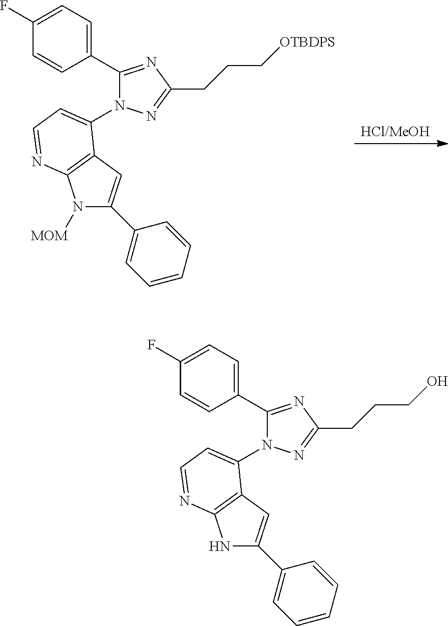

A mixture of 3-{3-[(tert-butyldiphenylsilyl)oxy]propyl}-5-(4-fluorophenyl)-1-[1-(methoxymethyl)-2-phenylpyrrolo[2,3-b]pyridin-4-yl]-1,2,4-triazole (60 mg, 0.086 mmol, 1.00 equiv) in MeOH (2 mL), concentrated HCl (2 mL) was stirred for overnight at 60° C. The reaction was quenched by the addition of water (30 mL) at RT and extracted with EtOAc (3×20 mL). The combined organic layers were washed with brine (2×20 mL), dried over anhydrous Na₂SO₄. After filtration, the filtrate was concentrated under reduced pressure. The residue was purified by reverse flash chromatography with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 40% B to 50% B in 8 min, 50% B; Wavelength: 254 nm; RT1 (min): 7 to afford Example 2 (9.6 mg, 26%) as a white solid. m/z (ESI+ve ion)=414.20 [M+H]⁺. H-NMR (400 MHz, DMSO-d₆) δ 12.6 (s, 1H), 8.41 (d, J=5.2 Hz, 1H), 8.17-8.13 (m, 2H), 7.99 (d, J=7.6 Hz, 1H), 7.49-7.46 (m, 2H), 7.40-7.33 (m, 4H), 7.00 (s, 1H), 4.51-4.49 (m, 1H), 3.46-3.41 (m, 2H), 2.97-2.93 (m, 2H), 1.93-1.86 (m, 2H).

### Example 4. 3-[3-(4-Fluorophenyl)-4-(2-phenyl-3H-imidazo[4,5-b]pyridin-7-yl)-1H-pyrazol-1-yl]propan-1-ol

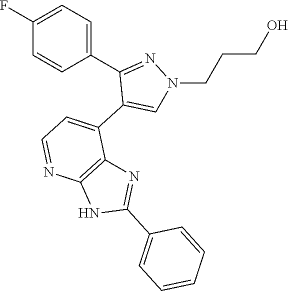 [SMILES: OCCCn1cc(-c2ccnc3[nH]c(-c4ccccc4)nc23)c(-c2ccc(F)cc2)n1]

### Step 1. 7-Bromo-2-phenyl-3H-imidazo[4,5-b]pyridine

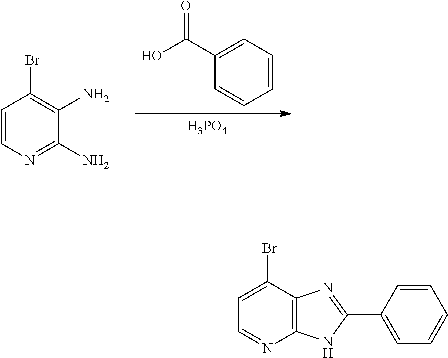

Polyphosphoric acid (10.0 g) was added to a mixture of 4-bromopyridine-2,3-diamine (500 mg, 2.53 mmol, 1.0 equiv.) and benzoic acid (338 mg, 2.75 mmol, 1.09 equiv.). The mixture was stirred for 16 hours at 130° C., then cooled to RT. Ice (25 mL) was added and the mixture was stirred vigorously for 30 minutes, until all the ice had melted. The resulting precipitate was filtered by suction filtration, while washing with water, to provide the title compound (715 mg, 98%) as a solid. ¹H NMR (400 MHz, CDCl₃) δ 8.30-8.23 (m, 2H), 8.21 (d, J=5.4 Hz, 1H), 7.62-7.56 (m, 3H), 7.53 (d, J=5.4 Hz, 1H). m/z (ESI, +ve ion)=274.0, 276.0 [M]⁺.

### Step 2. 7-(3-(4-Fluorophenyl)-1-(3-((triisopropylsilyl)oxy)propyl)-1H-pyrazol-4-yl)-2-phenyl-3H-imidazo[4,5-b]pyridine

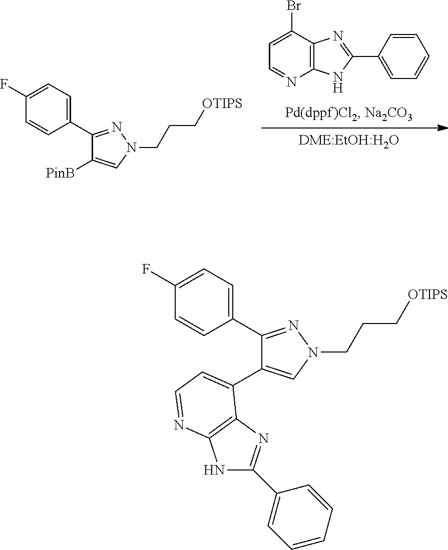

1,1′-Bis(diphenylphosphino)ferrocene dichloropalladium (II) (7.10 mg, 9.51 μmol, 0.1 equiv.) was added to a degassed mixture of 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 40.0 mg, 95.1 μmol), 7-bromo-2-phenyl-3H-imidazo[4,5-b]pyridine (26.1 mg, 95.1 μmol, 1.0 equiv.) and sodium carbonate (30.6 mg, 285 μmol, 3.0 equiv.) in a mixture of DME (490 μL), EtOH (280 μL) and H₂O (120 μL). The mixture was stirred for 30 min at 110° C. in a microwave reactor, then diluted with water (10 mL). The aqueous layer was extracted with EtOAc (3×15 mL). The combined organic layers were dried over Na₂SO₄ and concentrated under reduced pressure. The residue was purified by column chromatography (a gradient of EtOAc in hexanes, 0-100%, followed by MeOH in EtOAc, 0-15%), to provide the title compound (26.6 mg, 49%) as a solid. m/z (ESI, +ve ion)=570.3 [M+H]⁺.

### Step 3. 3-[3-(4-Fluorophenyl)-4-(2-phenyl-3H-imidazo[4,5-b]pyridin-7-yl)-1H-pyrazol-1-yl]propan-1-ol

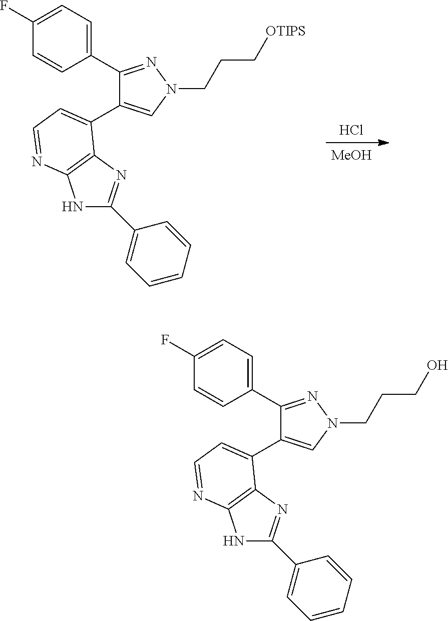

HCl (597 μL, 2.39 mmol, 4 M in dioxane, 40 equiv.) was added dropwise to a solution of 7-(3-(4-fluorophenyl)-1-(3-((triisopropylsilyl)oxy)propyl)-1H-pyrazol-4-yl)-2-phenyl-3H-imidazo[4,5-b]pyridine (40.0 mg, 59.7 μmol) in MeOH (2.00 mL) at RT. The mixture was stirred at RT for 15 minutes then concentrated under reduced pressure. The residue was purified by reverse phase chromatography (C18 column, MeCN in water with 10 mM ammonium formate, 5-60%, a gradient elution) to provide the title compound (5.30 mg, 21%) as a solid. ¹H NMR (400 MHz, DMSO-d₆) δ 13.53 (br s, 1H), 8.69 (s, 1H), 8.24 (d, J=7.0 Hz, 2H), 8.11 (d, J=4.7 Hz, 1H), 7.60-7.52 (m, 3H), 7.52-7.45 (m, 2H), 7.22 (t, J=8.1 Hz, 2H), 6.88 (d, J=4.8 Hz, 1H), 4.69 (t, J=4.6 Hz, 1H), 4.34 (t, J=6.7 Hz, 2H), 3.59-3.46 (m, 2H), 2.17-1.97 (m, 2H). m/z (ESI, +ve ion)=414.1 [M+H]⁺.

### Example 5. 3-[3-(4-Fluorophenyl)-4-(2-phenyl[1,3]oxazolo[5,4-d]pyrimidin-7-yl)-1H-pyrazol-1-yl]propan-1-ol

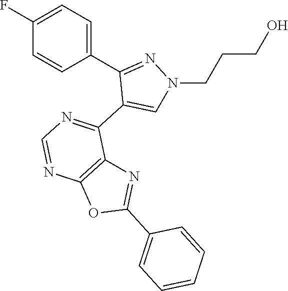 [SMILES: OCCCn1cc(-c2ncnc3oc(-c4ccccc4)nc23)c(-c2ccc(F)cc2)n1]

### Step 1. 7-(3-(4-Fluorophenyl)-1-(3-((triisopropylsilyl)oxy)propyl)-1H-pyrazol-4-yl)-2-phenyloxazolo[5,4-d]pyrimidine

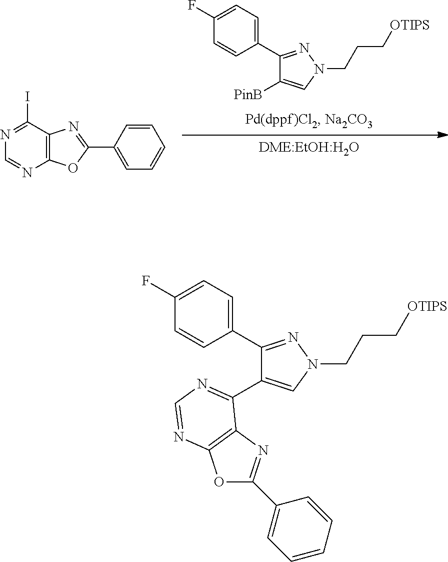

1,1′-Bis(diphenylphosphino)ferrocene dichloropalladium (II) (9.27 mg, 12.4 μmol, 0.1 equiv.) was added to a degassed mixture of 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 57.4 mg, 137 μmol, 1.1 equiv.), 7-iodo-2-phenyloxazolo[5,4-d]pyrimidine (Intermediate H, 40.0 mg, 124 μmol) and sodium carbonate (39.9 mg, 373 μmol, 3.0 equiv.) in a mixture of DME (640 μL), EtOH (370 μL) and H₂O (160 μL). The mixture was stirred for 30 minutes at 110° C. in a microwave reactor then diluted with water (10 mL). The aqueous layer was extracted with EtOAc (3×15 mL). The combined organic layers were washed with brine and concentrated under reduced pressure. The residue was purified by column chromatography (EtOAc in hexanes, 0-100%, a gradient elution) to provide the title compound (32.0 mg, 60%) as a solid. ¹H NMR (400 MHz, MeOD) δ 8.86 (s, 1H), 8.65 (s, 1H), 8.25-8.20 (m, 2H), 7.70-7.62 (m, 3H), 7.64-7.54 (m, 2H), 7.09 (dd, J=10.0, 7.8 Hz, 2H), 4.44 (t, J=6.6 Hz, 2H), 3.77 (t, J=5.9 Hz, 2H), 2.23-2.15 (m, 2H), 1.04 (s, 21H). m/z (ESI, +ve ion)=572.4 [M+H]⁺.

### Step 2. 3-[3-(4-Fluorophenyl)-4-(2-phenyl[1,3]oxazolo[5,4-d]pyrimidin-7-yl)-1H-pyrazol-1-yl]propan-1-ol

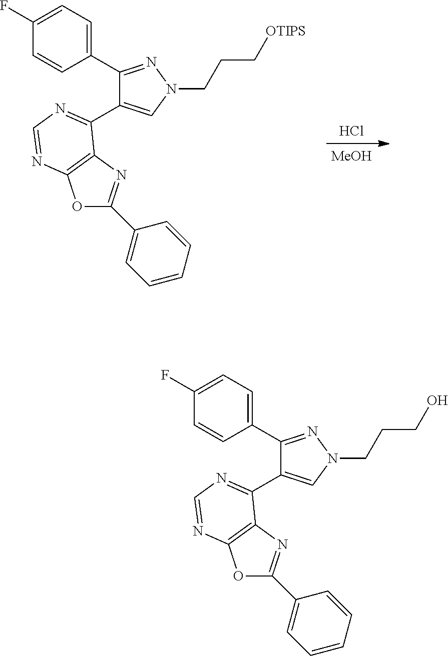

HCl (560 μL, 2.24 mmol, 4 M in dioxane, 40 equiv.) was added dropwise to a solution of 7-(3-(4-fluorophenyl)-1-(3-((triisopropylsilyl)oxy)propyl)-1H-pyrazol-4-yl)-2-phenyloxazolo[5,4-d]pyrimidine (32.0 mg, 56.0 μmol) in MeOH (1.88 mL) at RT. The mixture was stirred at RT for 10 minutes, then concentrated under reduced pressure. The residue was purified by reverse phase chromatography (C18 column, MeCN in water with 10 mM ammonium formate, 5-80%, a gradient elution) to provide the title compound (12.6 mg, 54%) as a solid. ¹H NMR (400 MHz, DMSO-d₆) δ 8.90 (s, 1H), 8.75 (s, 1H), 8.26-8.17 (m, 2H), 7.72-7.58 (m, 5H), 7.21-7.12 (m, 2H), 4.67 (t, J=5.1 Hz, 1H), 4.35 (t, J=7.1 Hz, 2H), 3.46 (dd, J=11.1, 6.0 Hz, 2H), 2.02 (p, J=6.4 Hz, 2H). m/z (ESI, +ve ion)=416.1 [M+H]⁺.

### Example 6. 3-[3-(4-Fluorophenyl)-4-(8-phenyl-9H-purin-6-yl)-1H-pyrazol-1-yl]propan-1-ol

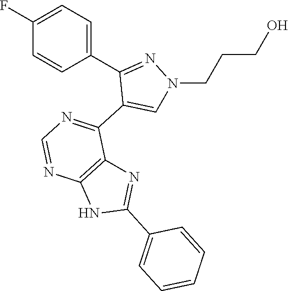 [SMILES: OCCCn1cc(-c2ncnc3[nH]c(-c4ccccc4)nc23)c(-c2ccc(F)cc2)n1]

### Step 1. 6-(3-(4-Fluorophenyl)-1-(3-((triisopropylsilyl)oxy)propyl)-1H-pyrazol-4-yl)-8-phenyl-9H-purine

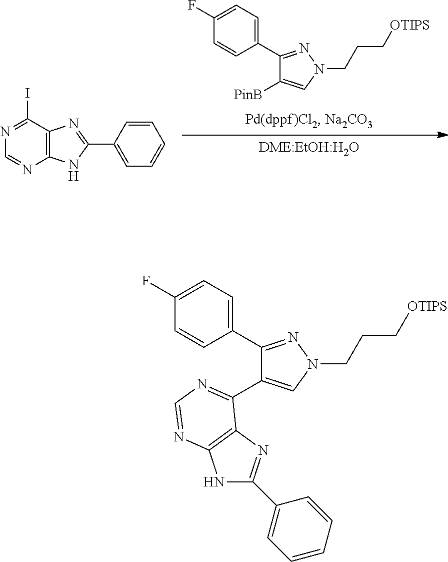

1,1′-Bis(diphenylphosphino)ferrocene dichloropalladium (II) (15.1 mg, 20.2 μmol, 0.1 equiv.) was added to a degassed mixture of 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 170 mg, 242 μmol, 1.2 equiv.), 6-iodo-8-phenyl-9H-purine (Intermediate G, 65.0 mg, 202 μmol) and sodium carbonate (64.8 mg, 605 μmol, 3.0 equiv.) in a mixture of DME (1.03 mL), EtOH (590 μL) and H₂O (260 μL). The mixture was stirred for 50 min at 110° C. in a microwave reactor then diluted with water (10 mL). The aqueous layer was extracted with EtOAc (3×15 mL). The combined organic layers were washed with brine and concentrated. The residue was purified by column chromatography (MeOH in DCM, 0-10%, a gradient elution) to provide the title compound (36.0 mg, 31%) as a solid. m/z (ESI, +ve ion)=571.4 [M+H]⁺.

### Step 2. 3-[3-(4-Fluorophenyl)-4-(8-phenyl-9H-purin-6-yl)-1H-pyrazol-1-yl]propan-1-ol

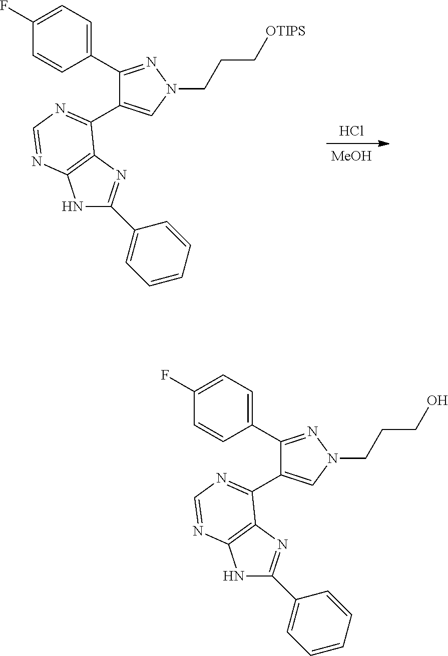

HCl (631 μL, 2.52 mmol, 4 M in dioxane, 40 equiv.) was added dropwise to a mixture of 6-(3-(4-fluorophenyl)-1-(3-((triisopropylsilyl)oxy)propyl)-1H-pyrazol-4-yl)-8-phenyl-9H-purine (36.0 mg, 63.1 μmol) in MeOH (2.11 mL) at RT. The mixture was stirred at RT for 15 minutes, then a solution of 15% w/w NaOH in water was added dropwise until pH 7-8. The aqueous layer was extracted with EtOAc (3×10 mL). The combined organic layers were washed with water, dried over Na2SO4, filtered, and concentrated under reduced pressure. The residue was purified by reverse phase chromatography (C18 column, MeCN in water with 10 mM ammonium formate, 10-100%, a gradient elution) to provide the title compound (18.0 mg, 69%) as a solid. ¹H NMR (400 MHz, DMSO-d₆) δ 8.97 (s, 1H), 8.64 (s, 1H), 8.32-8.25 (m, 2H), 7.73-7.67 (m, 2H), 7.63-7.55 (m, 3H), 7.21-7.14 (m, 2H), 4.68 (s, 1H), 4.37 (t, J=7.1 Hz, 2H), 3.51 (t, J=6.0 Hz, 2H), 2.06 (p, J=6.4 Hz, 2H). m/z (ESI, +ve ion)=415.2 [M+H]⁺.

### Example 7. 3-[3-(4-Fluorophenyl)-4-(2-phenyl-1H-pyrrolo[3,2-b]pyridin-7-yl)-1H-pyrazol-1-yl]propan-1-ol

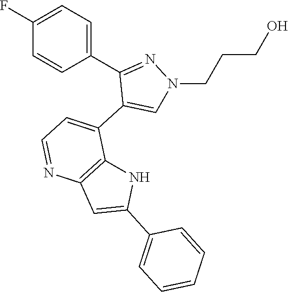 [SMILES: OCCCn1cc(-c2ccnc3cc(-c4ccccc4)[nH]c23)c(-c2ccc(F)cc2)n1]

### Step 1. 4-Chloro-2-(phenylethynyl)pyridin-3-amine

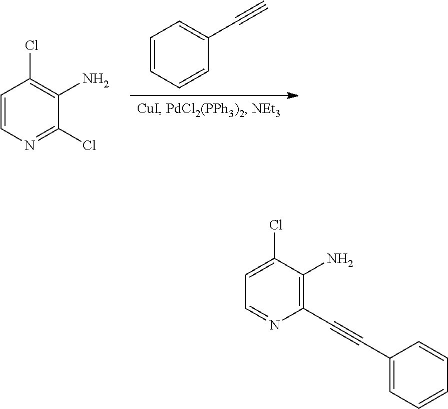

3-Amino-2,4-dichloropyridine (853 μL, 6.13 mmol), triethylamine (30.6 mL, 220 mmol, 24 equiv.), copper(I) iodide (59.6 mg, 307 μmol, 0.05 equiv.) and bis(triphenylphosphine)palladium(II) dichloride (216 mg, 307 μmol, 0.05 equiv.). The vial was sealed, purged with nitrogen, and evacuated three times and then cooled to 0° C. Phenylacetylene (1.01 mL, 9.20 mmol, 1.5 equiv.) was added, the mixture was warmed to RT, heated at 80° C. for 5 h and then cooled to RT for 11 h. The mixture was filtered and the filtrate was concentrated under reduced pressure. The residue was purified by column chromatography (EtOAc in hexanes, 3-20%, a gradient elution) to provide the title compound (600 mg, 43%) as a solid. ¹H NMR (400 MHz, DMSO-d₆) δ 7.76 (d, J=5.0 Hz, 1H), 7.74-7.67 (m, 2H), 7.49-7.42 (m, 3H), 7.35 (d, J=5.1 Hz, 1H), 5.86 (s, 2H). m/z (ESI, +ve ion)=229.0, 231.0 [M+H]⁺.

### Step 2. 7-Chloro-2-phenyl-1H-pyrrolo[3,2-b]pyridine

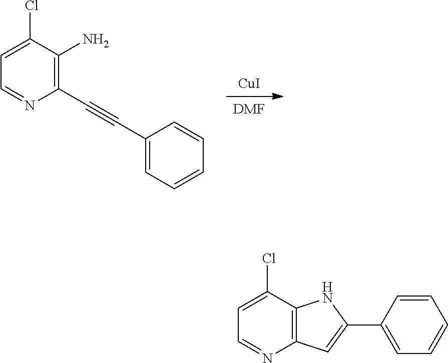

Copper (I) iodide (23.2 mg, 119 μmol, 0.05 equiv.) was added to a solution of 4-chloro-2-(phenylethynyl)pyridin-3-amine (545 mg, 2.38 mmol) and DMF (23.8 mL). The mixture was heated at 100° C. for 3.5 hours. Additional copper (I) iodide (40.0 mg, 206 μmol, 0.09 equiv.) was added and the reaction was further heated for 20 hours. The reaction mixture was cooled to RT and poured into water (30 mL) and extracted with EtOAc (30 mL×3). The combined organic layer was washed with additional water, brine, dried over Na₂SO₄, filtered, and concentrated under reduced pressure. The residue was purified by column chromatography (EtOAc in hexanes, 3-20%, a gradient elution) to provide the title compound (220 mg, 40%) as a solid. ¹H NMR (400 MHz, DMSO-d₆) δ 11.96 (s, 1H), 8.26 (d, J=5.1 Hz, 1H), 8.05 (dd, J=5.2, 3.4 Hz, 2H), 7.51 (dd, J=10.4, 4.8 Hz, 2H), 7.42 (dt, J=9.2, 4.3 Hz, 1H), 7.24 (d, J=5.1 Hz, 1H), 7.16 (d, J=2.2 Hz, 1H). m/z (ESI, +ve ion)=229.0, 231.0 [M+H]⁺.

### Step 3. 7-(3-(4-Fluorophenyl)-1-(3-((triisopropylsilyl)oxy)propyl)-1H-pyrazol-4-yl)-2-phenyl-1H-pyrrolo[3,2-b]pyridine

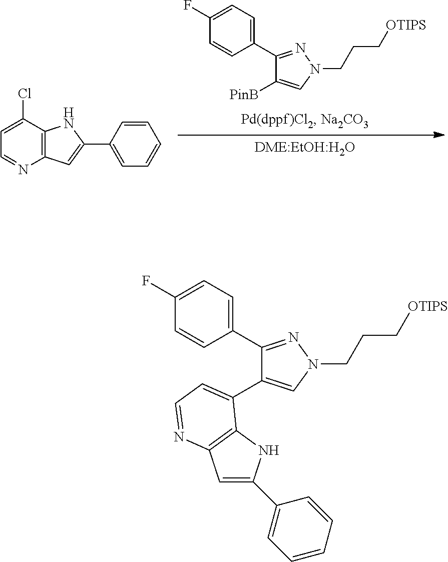

1,1′-Bis(diphenylphosphino)ferrocene dichloropalladium (II) (28.8 mg, 39.4 μmol, 0.1 equiv.) was added to a degassed mixture of 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 218 mg, 433 μmol, 1.1 equiv.), 7-chloro-2-phenyl-1H-pyrrolo[3,2-b]pyridine (90.0 mg, 394 μmol) and sodium carbonate (126 mg, 1.18 mmol, 3.0 equiv.) in a mixture of DME (2.2 mL), EtOH (1.1 mL) and H₂O (550 μL). The mixture was stirred for 1 hour at 110° C. in a microwave reactor then diluted with water (20 mL). The aqueous layer was extracted with EtOAc (3×20 mL). The combined organic layers were washed with brine and concentrated under reduced pressure. The residue was purified by column chromatography (MeOH in DCM, 1-6%, a gradient elution), to provide the title compound (32.0 mg, 60%) as a solid. ¹H NMR (400 MHz, CDCl₃) δ 8.39 (d, J=5.2 Hz, 1H), 7.84 (s, 1H), 7.54-7.46 (m, 2H), 7.43-7.36 (m, 3H), 7.28 (dd, J=7.8, 1.8 Hz, 2H), 7.20-7.14 (m, 1H), 7.12 (s, 1H), 7.03 (dd, J=11.8, 5.5 Hz, 2H), 4.44 (t, J=6.9 Hz, 2H), 3.81 (t, J=5.7 Hz, 2H), 2.29-2.18 (m, 2H), 1.12-1.00 (m, 21H). m/z (ESI, +ve ion)=569.1 [M+H]⁺.

### Step 4. 3-[3-(4-Fluorophenyl)-4-(2-phenyl-1H-pyrrolo[3,2-b]pyridin-7-yl)-1H-pyrazol-1-yl]propan-1-ol

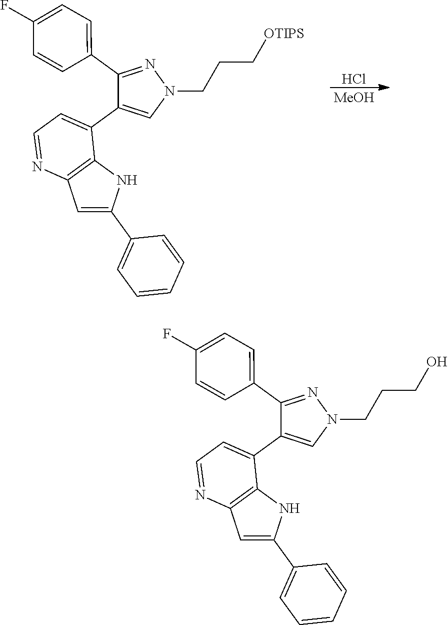

HCl (2.17 mL, 37% w/w in water) was slowly added to a solution of 7-(3-(4-fluorophenyl)-1-(3-((triisopropylsilyl)oxy)propyl)-1H-pyrazol-4-yl)-2-phenyl-1H-pyrrolo[3,2-b]pyridine (37.0 mg, 65.0 μmol) in MeOH (2.17 mL) at RT. The mixture was stirred for 17 h at RT. A 15% w/w NaOH solution in water was added until pH 7-8. The mixture was extracted with EtOAc (3×20 mL). The combined organic layer was dried over Na₂SO₄, filtered, and concentrated under reduced pressure. The crude material was purified by reverse phase chromatography (C18 column, MeCN in water with 10 mM ammonium formate, 5-70%, a gradient elution) to provide the title compound (22.1 mg, 87%) as a solid. ¹H NMR (400 MHz, DMSO-d₆) δ 11.10 (s, 1H), 8.23 (d, J=4.8 Hz, 1H), 8.19 (s, 1H), 8.17 (s, 1H), 7.89-7.84 (m, 2H), 7.47-7.38 (m, 4H), 7.38-7.32 (m, 1H), 7.13-7.07 (m, 2H), 7.05 (s, 1H), 6.80 (d, J=4.8 Hz, 1H), 4.30 (t, J=7.2 Hz, 2H), 3.55 (t, J=6.1 Hz, 2H), 2.14-2.02 (m, 2H). m/z (ESI, +ve ion)=413.3 [M+H]⁺.

### Example 8. 3-[4-(4-Fluorophenyl)-5-(2-phenyl-1H-pyrrolo[2,3-b]pyridin-4-yl)-1,3-thiazol-2-yl]propan-1-01

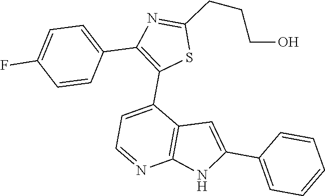 [SMILES: OCCCc1nc(-c2ccc(F)cc2)c(-c2ccnc3[nH]c(-c4ccccc4)cc23)s1]

### Step 1. 4-[(tert-Butyldiphenylsilyl)oxy]butanethioamide

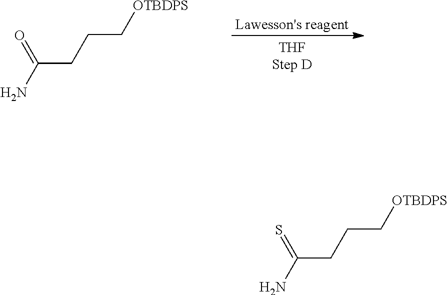

To a stirred solution of 4-[(tert-butyldiphenylsilyl)oxy]butanamide (1 g, 2.93 mmol, 1.00 equiv) in THF (10 mL) was added Lawesson Reagent (0.59 g, 1.46 mmol, 0.5 equiv) in portions at room temperature under nitrogen atmosphere. The resulting mixture was stirred for 1 h at room temperature under nitrogen atmosphere then was quenched by the addition of water (70 mL) at room temperature. The resulting mixture was extracted with EtOAc (3×70 mL). The combined organic layers were washed with brine (2×50 mL), dried over anhydrous Na2SO4. After filtration, the filtrate was concentrated under reduced pressure. The residue was purified by silica gel column chromatography, eluted with PE:EtOAc (3:1) to afford the title compound (900 mg, 86%) as a yellow solid. m/z (ESI, +ve ion)=358.30 [M+H]⁺.

### Step 2. 3-[4-(4-Fluorophenyl)-1,3-thiazol-2-yl]propan-1-ol

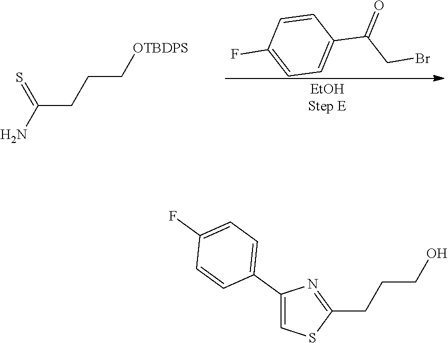

To a stirred solution of 4-[(tert-butyldiphenylsilyl)oxy]butanethioamide (1 g, 2.80 mmol, 1.00 equiv) in EtOH (10 mL) was added 2-bromo-1-(4-fluorophenyl)ethanone (0.61 g, 2.80 mmol, 1 equiv) at room temperature under nitrogen atmosphere. The resulting mixture was stirred for overnight at 70° C. under nitrogen atmosphere then cooled down to room temperature. The reaction was quenched by the addition of water (100 mL) and extracted with EtOAc (3×100 mL). The combined organic layers were washed with brine (100 mL), dried over anhydrous Na₂SO₄. After filtration, the filtrate was concentrated under reduced pressure. The residue was purified by silica gel column chromatography, eluted with PE:EtOAc (1:1) to afford the title compound (522 mg, 79%) as a yellow oil. m/z (ESI, +ve ion)=238.05 [M+H]⁺.

### Step 3. 2-{3-[(tert-Butyldiphenylsilyl)oxy]propyl}-4-(4-fluorophenyl)-1,3-thiazole

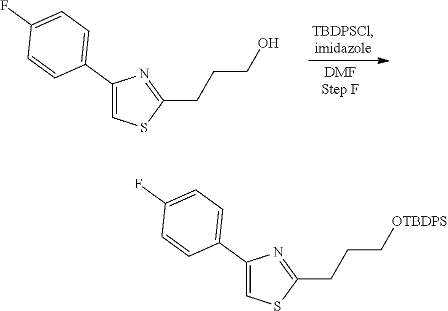

To a stirred solution of 3-[4-(4-fluorophenyl)-1,3-thiazol-2-yl]propan-1-ol (520 mg, 2.19 mmol, 1.00 equiv) and Imidazole (298 mg, 4.38 mmol, 2 equiv) in DMF (6 mL) was added TBDPSCl (663 mg, 2.41 mmol, 1.1 equiv) dropwise at room temperature under nitrogen atmosphere. The resulting mixture was stirred for 16 h at room temperature under nitrogen atmosphere then quenched by the addition of water (60 mL) at room temperature. The resulting mixture was extracted with EtOAc (3×60 mL). The combined organic layers were washed with brine (60 mL), dried over anhydrous Na₂SO₄. After filtration, the filtrate was concentrated under reduced pressure. The residue was purified by silica gel column chromatography, eluted with PE:EtOAc (1:1) to afford the title compound (925 mg, 89%) as a yellow oil. m/z (ESI, +ve ion)=476.25 [M+H]⁺.

### Step 4. 2-{3-[(tert-Butyldiphenylsilyl)oxy]propyl}-4-(4-fluorophenyl)-5-(tributylstannyl)-1,3-thiazole

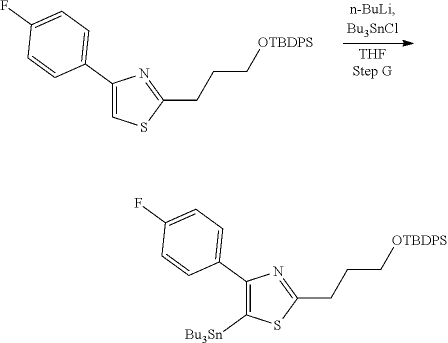

To a stirred solution of 2-{3-[(tert-butyldiphenylsilyl)oxy]propyl}-4-(4-fluorophenyl)-1,3-thiazole (48 mg, 0.101 mmol, 1.00 equiv) and n-BuLi (0.12 mL, 0.303 mmol, 3 equiv, 2.5 M in hexane) in THF (0.6 mL) was added SnBu₃Cl (98.54 mg, 0.303 mmol, 3 equiv) dropwise at −78° C. under nitrogen atmosphere. The resulting mixture was stirred for 1.5 h at −78° C. under nitrogen atmosphere. The crude product (57 mg, 52%) was used in the next step directly without further purification. m/z (ESI+ve ion)=766.35 [M+H]⁺.

### Step 5. 5-[1-(Benzenesulfonyl)-2-phenylpyrrolo[2,3-b]pyridin-4-yl]-2-{3-[(tert-butyldiphenylsilyl)oxy]propyl}-4-(4-fluorophenyl)-1,3-thiazole

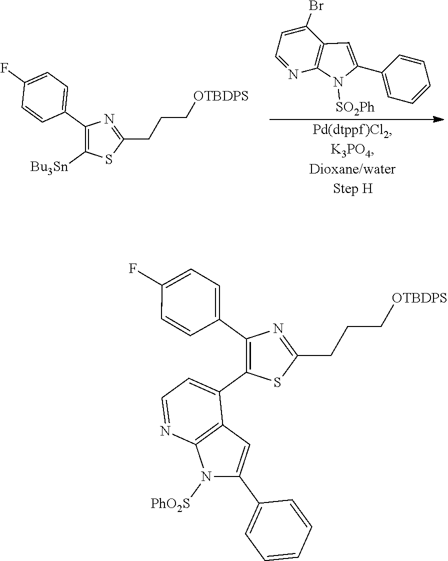

To a stirred solution of 2-{3-[(tert-butyldiphenylsilyl)oxy]propyl}-4-(4-fluorophenyl)-5-(tributylstannyl)-1,3-thiazole (860 mg, 1.13 mmol, 1.00 equiv) and 1-(benzenesulfonyl)-4-bromo-2-phenylpyrrolo[2,3-b]pyridine (Intermediate E, 697 mg, 1.69 mmol, 1.5 equiv) in dioxane (10 mL) were added Pd(DtBPF)Cl₂ (147 mg, 0.225 mmol, 0.2 equiv) and K₃PO₄ (477 mg, 2.25 mmol, 2 equiv) at room temperature under nitrogen atmosphere. The resulting mixture was stirred for 2 h at 90° C. under nitrogen atmosphere. The mixture was allowed to cool down to room temperature and then quenched by the addition of water (150 mL). The resulting mixture was extracted with EtOAc (3×100 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (2/1) to afford the title compound (720 mg, 79%) as a yellow solid. m/z (ESI+ve ion)=808.35 [M+H]⁺.

### Step 6. 3-[4-(4-Fluorophenyl)-5-{2-phenyl-1H-pyrrolo[2,3-b]pyridin-4-yl}-1,3-thiazol-2-yl]propan-1-ol

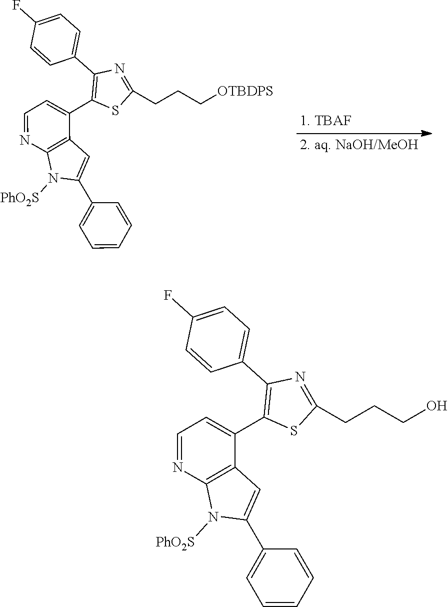

To a stirred solution of 5-[1-(benzenesulfonyl)-2-phenylpyrrolo[2,3-b]pyridin-4-yl]-2-{3-[(tert-butyldiphenylsilyl)oxy]propyl}-4-(4-fluorophenyl)-1,3-thiazole (200 mg, 0.351 mmol, 1.00 equiv) in THF (5 mL) was added TBAF (1.5 mL, 1 M in THF) at room temperature under nitrogen atmosphere. The resulting mixture was stirred for 3 h at 40° C. under nitrogen atmosphere then quenched by the addition of water (5 mL) at room temperature. The resulting mixture was extracted with EtOAc (3×10 mL). The combined organic layers were washed with brine (2×10 mL), dried over anhydrous Na₂SO₄. After filtration, the filtrate was concentrated under reduced pressure. To the above residue was added MeOH (0.5 mL) and NaOH (10 M, 0.06 mL) dropwise over 2 min at room temperature. The resulting mixture was stirred for additional 1 h at 60° C. then quenched by the addition of water/Ice (5 mL) at room temperature. The resulting mixture was extracted with EtOAc (3×10 mL). The combined organic layers were washed with brine (10 mL), dried over anhydrous Na₂SO₄. After filtration, the filtrate was concentrated under reduced pressure. The crude product (68 mg) was purified by prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 40% B to 50% B in 8 min, 50% B; Wavelength: 254 nm; RT1 (min): 7.12 to afford Example 8 (35 mg, 23%) as a yellow solid. m/z (ESI+ve ion)=430.15 [M+H]⁺. ¹H NMR (400 MHz, Methanol-d₄) δ 8.16 (d, J=5.04 Hz, 1H), 7.74-7.72 (m, 2H), 7.52-7.43 (m, 4H), 7.38-7.34 (m, 1H), 7.05-7.00 (m, 3H), 6.50 (s, 1H), 3.77-3.74 (m, 2H), 3.25-3.21 (m, 2H), 2.18-2.11 (m, 2H).

### Example 9. 3-[3-(4-Fluorophenyl)-4-(6-phenyl-7H-pyrrolo[2,3-d]pyrimidin-4-yl)-1H-pyrazol-1-yl]propan-1-ol

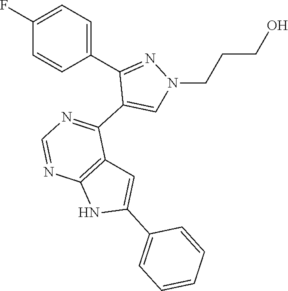 [SMILES: OCCCn1cc(-c2ncnc3[nH]c(-c4ccccc4)cc23)c(-c2ccc(F)cc2)n1]

### Step 1. 4-Chloro-6-phenyl-7H-pyrrolo[2,3-d]pyrimidine

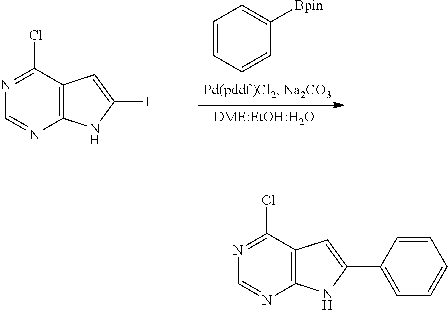

Phenylboronic acid pinacol ester (515 mg, 2.40 mmol, 1.1 equiv.), sodium carbonate (223 mg, 2.09 mmol, 1.0 equiv.), and 4-chloro-6-iodo-7H-pyrrolo[2,3-d]pyrimidine (595 mg, 2.09 mmol) were suspended in DME (7.59 mL), EtOH (3.25 mL) and H₂O (1.08 mL). The mixture was degassed with nitrogen for 5 minutes, then 1,1′-bis(diphenylphosphino)ferrocene dichloropalladium (II) (76.3 mg, 104 μmol, 0.05 equiv.) was added and the mixture was further degassed with nitrogen for 5 minutes before being heated to 120° C. in a microwave reactor for 90 minutes. The mixture was heated at 130° C. in a microwave reactor for an additional 90 minutes. The mixture was cooled to room temperature, diluted with EtOAc (20 mL), filtered and the filtrate was concentrated under reduced pressure. The residue was purified by column chromatography (EtOAc in hexanes, 10-100%, a gradient elution) to provide the title compound (317 mg, 66%) as a solid. m/z (ESI, +ve ion)=230.0, 232.0 [M+H]⁺.

### Step 2. 4-Iodo-6-phenyl-7H-pyrrolo[2,3-d]pyrimidine

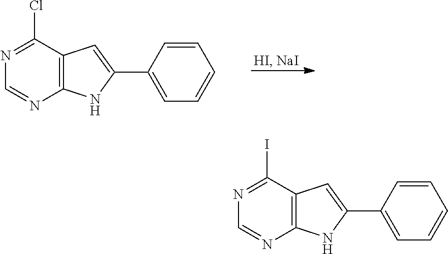 [SMILES: Clc1ncnc2[nH]c(-c3ccccc3)cc12 ; Ic1ncnc2[nH]c(-c3ccccc3)cc12]

4-Chloro-6-phenyl-7H-pyrrolo[2,3-d]pyrimidine (200 mg, 871 μmol) and sodium iodide (527 mg, 3.48 mmol, 4.0 equiv.) were suspended in hydriodic acid (2.97 mL, 13.1 mmol, 15 equiv.). The resulting mixture was stirred at 60° C. for 16 h, then mixture was further heated to 90° C. for 16 h. The reaction mixture was diluted with ice/water (15 mL). The mixture was stirred vigorously until all of the ice had melted. 15% w/w NaOH solution in water was added until a pH of 6-7 was obtained. EtOAc (30 mL) was added and the layers were separated. The aqueous layer was extracted with additional EtOAc (3×25 mL) and the combined organic layers were washed with water (15 mL), brine (15 mL), dried over Na₂SO₄, filtered, and concentrated under reduced pressure to provide the title compound (193 mg, 69%) as a solid. m/z (ESI, +ve ion)=322.0 [M+H]⁺.

### Step 3. 4-(3-(4-Fluorophenyl)-1-(3-((triisopropylsilyl)oxy)propyl)-1H-pyrazol-4-yl)-6-phenyl-7H-pyrrolo[2,3-d]pyrimidine

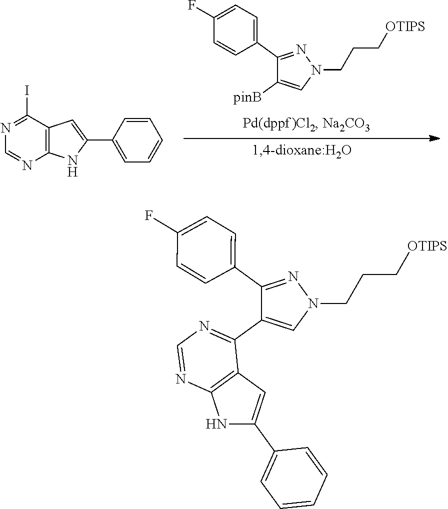

1,1′-Bis(diphenylphosphino)ferrocene dichloropalladium (II) (34.9 mg, 46.7 μmol, 0.07 equiv.) was added to a degassed mixture of 4-iodo-6-phenyl-7H-pyrrolo[2,3-d]pyrimidine (352 mg, 701 μmol), 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 352 mg, 701 μmol, 1.0 equiv.), and sodium carbonate (150 mg, 1.40 mmol, 2.0 equiv.) in a mixture of 1,4-dioxane (10.4 mL) and H₂O (1.38 mL), and the resulting mixture was degassed using nitrogen for additional 5 minutes. The mixture was stirred for 16 hours at 90° C., cooled to room temperature, diluted with EtOAc, and filtered. The filtrate was concentrated under reduced pressure. The residue was purified by column chromatography (EtOAc in hexanes, 0-60%, a gradient elution), to provide the title compound (266 mg, 99%) as a solid. m/z (ESI, +ve ion)=570.6 [M+H]⁺.

### Step 4. 3-[3-(4-Fluorophenyl)-4-(6-phenyl-7H-pyrrolo[2,3-d]pyrimidin-4-yl)-1H-pyrazol-1-yl]propan-1-ol

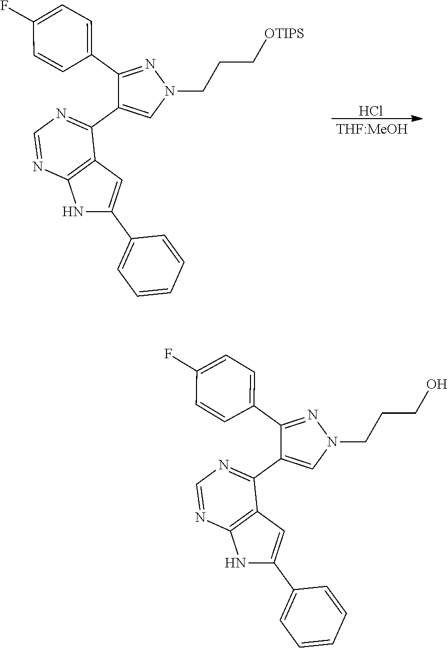

Hydrochloric acid (467 μL, 934 μmol, 2 M in water, 2.0 equiv.) was added dropwise to a solution of 4-(3-(4-fluorophenyl)-1-(3-((triisopropylsilyl)oxy)propyl)-1H-pyrazol-4-yl)-6-phenyl-7H-pyrrolo[2,3-d]pyrimidine (266 mg, 476 μmol) in a mixture of THF (1.00 mL) and MeOH (1.00 mL), and the mixture was stirred at room temperature for 16 h. The resulting precipitate was filtered by suction filtration, washed with THF, and allowed to dry under vacuum to provide the title compound (88.4 mg, 46%) as a solid. ¹H NMR (400 MHz, CD₃OD) δ 8.89 (s, 1H), 8.54 (s, 1H), 7.80-7.70 (m, 2H), 7.62-7.44 (m, 5H), 7.21-7.09 (m, 2H), 6.72 (s, 1H), 4.49 (t, J=6.9 Hz, 2H), 3.68 (t, J=6.0 Hz, 2H), 2.23 (p, J=6.5 Hz, 2H). m/z (ESI, +ve ion)=414.2 [M+H]⁺.

### Example 10. 3-[3-(4-Fluorophenyl)-4-{2-[4-(4-methylpiperazin-1-yl)phenyl][1,3]oxazolo[5,4-d]pyrimidin-7-yl}-1H-pyrazol-1-yl]propan-1-ol

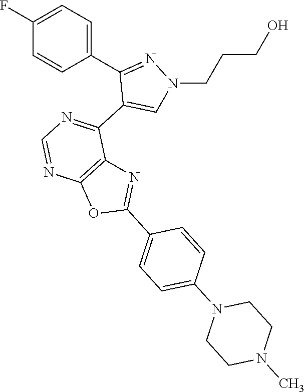 [SMILES: CN1CCN(c2ccc(-c3nc4c(-c5cn(CCCO)nc5-c5ccc(F)cc5)ncnc4o3)cc2)CC1]

### Step 1. 4-(4-Methylpiperazin-1-yl)benzoyl chloride

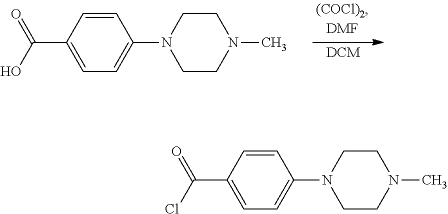 [SMILES: CN1CCN(c2ccc(C(=O)O)cc2)CC1 ; CN1CCN(c2ccc(C(=O)Cl)cc2)CC1]

A solution of 4-(4-methylpiperazin-1-yl)benzoic acid (1.00 g, 4.54 mmol) in DCM (9.08 mL) was cooled to 0° C., then DMF (1.00 mL) and oxalyl chloride (784 μL, 9.08 mmol, 2 equiv.) were added sequentially. The reaction was allowed to stir at room temperature for 1 h then concentrated under reduced pressure to give the title compound (1.40 g, quant.) as a solid. ¹H NMR (400 MHz, CDCl₃) δ 8.08 (d, J=8.8 Hz, 2H), 7.10 (d, J=7.9 Hz, 2H), 4.07-4.01 (m, 2H), 3.92-3.89 (m, 2H), 3.65-3.62 (m, 2H), 3.23-3.21 (m, 2H), 2.90 (d, J=4.1 Hz, 3H).

### Step 2. 4-(4-Methylpiperazin-1-yl)benzoyl chloride

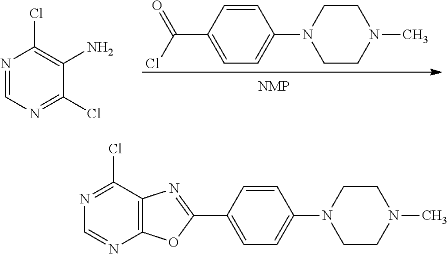

4,6-Dichloropyrimidin-5-amine (421 mg, 2.51 mmol, 1.2 equiv.) and 4-(4-methylpiperazin-1-yl)benzoyl chloride (500 mg, 2.09 mmol) were suspended in NMP (420 μL) and the reaction was heated in a microwave reactor at 150° C. for 1.5 h. The residue was directly purified by column chromatography (MeOH in DCM, 1-20%, a gradient elution). The resulting solid was triturated in DCM and collected by suction filtration to provide the title compound (260 mg, 38%) as a solid. m/z (ESI, +ve ion)=330.1 [M+H]⁺.

### Step 3. 4-(4-Methylpiperazin-1-yl)benzoyl chloride

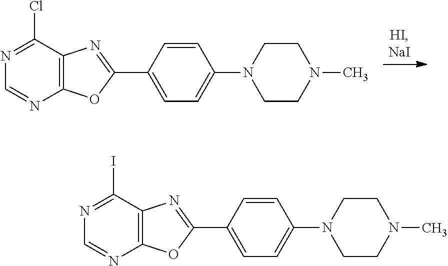 [SMILES: CN1CCN(c2ccc(-c3nc4c(Cl)ncnc4o3)cc2)CC1 ; CN1CCN(c2ccc(-c3nc4c(I)ncnc4o3)cc2)CC1]

4-(4-Methylpiperazin-1-yl)benzoyl chloride (120 mg, 364 μmol) and sodium iodide (73.8 mg, 488 μmol, 1.3 equiv.) were suspended in hydriodic acid (241 μL, 1.82 mmol, 5 equiv.) and heated at 60° C. for 2 hours. The mixture was cooled to room temperature then diluted with ice/water (2 mL) and 2 M NaOH solution (2 mL). The mixture was stirred vigorously until all of the ice had melted. EtOAc (5 mL) and water (2 mL) were added, and the layers were separated. The aqueous layer was extracted with additional EtOAc (3×5 mL). The combined organic layers were washed with water (5 mL), dried over Na₂SO₄, filtered, and concentrated under reduced pressure to provide the title compound (93.0 mg, 47%) as a solid. m/z (ESI, +ve ion)=422.1 [M+H]⁺.

### Step 4. 7-(3-(4-Fluorophenyl)-1-(3-((triisopropylsilyl)oxy)propyl)-1H-pyrazol-4-yl)-2-(4-(4-methylpiperazin-1-yl)phenyl)oxazolo[5,4-d]pyrimidine

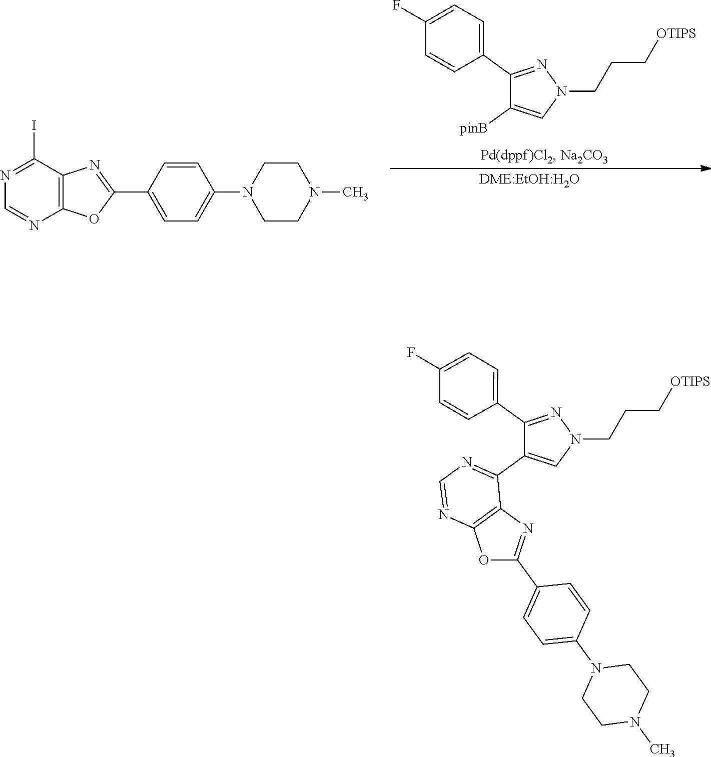

1,1′-Bis(diphenylphosphino)ferrocene dichloropalladium (II) (15.6 mg, 21.4 μmol, 0.1 equiv.) was added to a degassed mixture of 4-(4-methylpiperazin-1-yl)benzoyl chloride (90.0 mg, 214 μmol), 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 118 mg, 235 μmol, 1.1 equiv.), and sodium carbonate (68.6 mg, 641 μmol, 3.0 equiv.) in a mixture of DME (1.20 mL), EtOH (600 μL) and H₂O (300 μL), and the resulting mixture was degassed using nitrogen and evacuated three times. The mixture was stirred for 1 hour at 110° C. in a microwave reactor, then diluted with water. The aqueous mixture was extracted with EtOAc (3×20 mL). The combined organic layers were washed with brine, dried over Na₂SO₄, filtered, and concentrated under reduced pressure. The residue was purified by column chromatography (MeOH in DCM, 1-10%, a gradient elution) to provide the title compound (62.0 mg, 43%) as a solid. ¹H NMR (400 MHz, CDCl₃) δ 8.74 (s, 1H), 8.70 (s, 1H), 8.11 (d, J=9.0 Hz, 2H), 7.75-7.68 (m, 2H), 7.10-7.04 (m, 2H), 6.98 (d, J=9.1 Hz, 2H), 4.44 (t, J=6.8 Hz, 2H), 3.78 (t, J=5.7 Hz, 2H), 3.46 (s, 4H), 2.65 (d, J=1.5 Hz, 4H), 2.42 (s, 3H), 2.26-2.16 (m, 2H), 1.08-1.03 (m, 21H). m/z (ESI, +ve ion)=670.3 [M+H]⁺.

### Step 5. 3-[3-(4-Fluorophenyl)-4-{2-[4-(4-methylpiperazin-1-yl)phenyl][1,3]oxazolo[5,4-d]pyrimidin-7-yl}-1H-pyrazol-1-yl]propan-1-ol

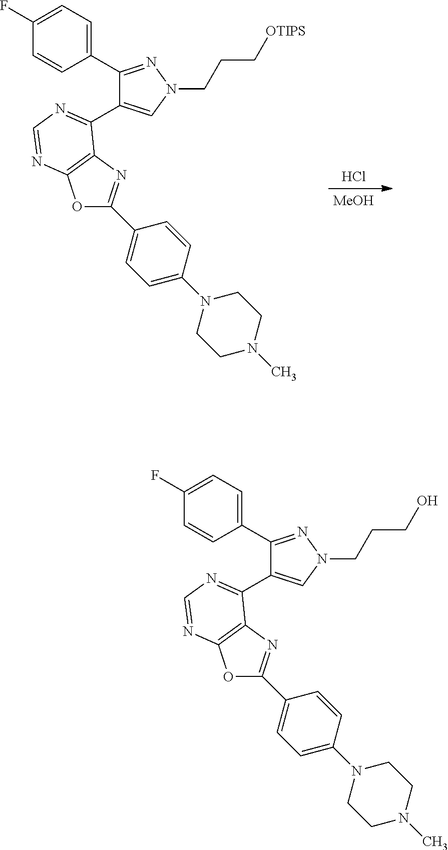

Hydrochloric acid (1.01 mL, 33.3 mmol, 37 wt. % in water, 406 equiv.) was added dropwise to a solution of 7-(3-(4-fluorophenyl)-1-(3-((triisopropylsilyl)oxy)propyl)-1H-pyrazol-4-yl)-2-(4-(4-methylpiperazin-1-yl)phenyl)oxazolo[5,4-d]pyrimidine (55.0 mg, 82.1 μmol) in MeOH (3.74 mL) at room temperature. The mixture was stirred at room temperature for 10 minutes. A 15% w/w aq. NaOH solution in water was added until pH 7-8. The mixture was extracted with EtOAC (3×20 mL). The combined organic layer was dried over Na₂SO₄, filtered, and concentrated under reduced pressure. The residue was purified by reverse phase chromatography (C18 column, MeCN in water with 10 mM ammonium formate, 5-70%, a gradient elution) to provide the title compound (25.2 mg, 60%) as a solid. ¹H NMR (400 MHz, DMSO-d₆) δ 8.87 (s, 1H), 8.68 (s, 1H), 8.04 (d, J=9.1 Hz, 2H), 7.71-7.65 (m, 2H), 7.23-7.10 (m, 4H), 4.69 (br s, 1H), 4.38 (t, J=7.0 Hz, 2H), 3.50 (t, J=6.1 Hz, 2H), 3.39-3.37 (m, 4H), 2.49-2.42 (m, 4H), 2.23 (s, 3H), 2.05 (p, J=6.4 Hz, 2H). m/z (ESI, +ve ion)=514.3 [M+H]⁺.

### Example 11. 3-[3-(4-Fluorophenyl)-4-(6-phenylfuro[2,3-d]pyrimidin-4-yl)-1H-pyrazol-1-yl]propan-1-ol

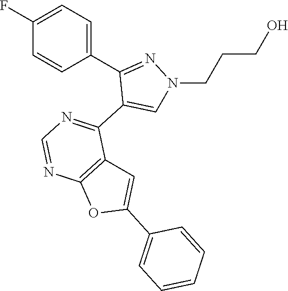 [SMILES: OCCCn1cc(-c2ncnc3oc(-c4ccccc4)cc23)c(-c2ccc(F)cc2)n1]

### Step 1. 4-(3-(4-Fluorophenyl)-1-(3-((triisopropylsilyl)oxy)propyl)-1H-pyrazol-4-yl)-6-phenylfuro[2,3-d]pyrimidine

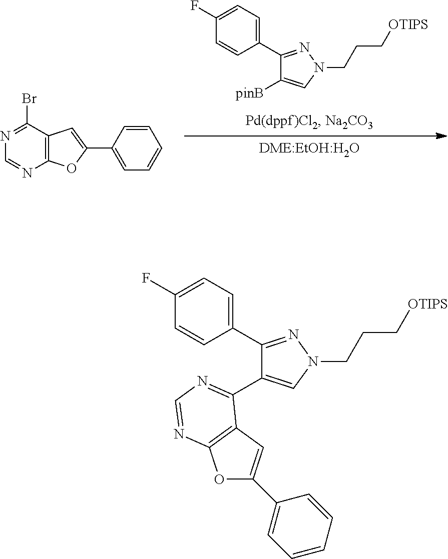

1,1′-Bis(diphenylphosphino)ferrocene dichloropalladium (II) (12.2 mg, 16.4 μmol, 0.1 equiv.) was added to a degassed mixture of 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 82d mg, 164 μmol, 1.0 equiv.), 4-bromo-6-phenylfuro[2,3-d]pyrimidine (Intermediate I, 45 mg, 164 μmol) and sodium carbonate (52.5 mg, 491 μmol, 3.0 equiv.) in a mixture of DME (840 μL), EtOH (480 μL) and H₂O (210 μL). The reaction mixture was stirred for 40 minutes at 110° C. in a microwave reactor, then silica gel was added. The resulting mixture was concentrated under reduced pressure. The dry-loaded residue was purified by column chromatography (EtOAc in hexanes, 0-50%, a gradient elution), to provide the title compound (45.0 mg, 48%) as a solid. ¹H NMR (400 MHz, CDCl₃) δ 8.91 (s, 1H), 8.19 (s, 1H), 7.62 (d, J=7.9 Hz, 2H), 7.53 (dd, J=7.2, 5.4 Hz, 2H), 7.48-7.37 (m, 3H), 7.08 (dd, J=12.3, 4.9 Hz, 2H), 6.09 (s, 1H), 4.42 (t, J=6.9 Hz, 2H), 3.81 (t, J=5.3 Hz, 2H), 2.22 (p, J=6.7 Hz, 2H), 1.07 (s, 21H). m/z (ESI, +ve ion)=571.5 [M+H]⁺.

### Step 2. 3-[3-(4-Fluorophenyl)-4-(6-phenylfuro[2,3-d]pyrimidin-4-yl)-1H-pyrazol-1-yl]propan-1-ol

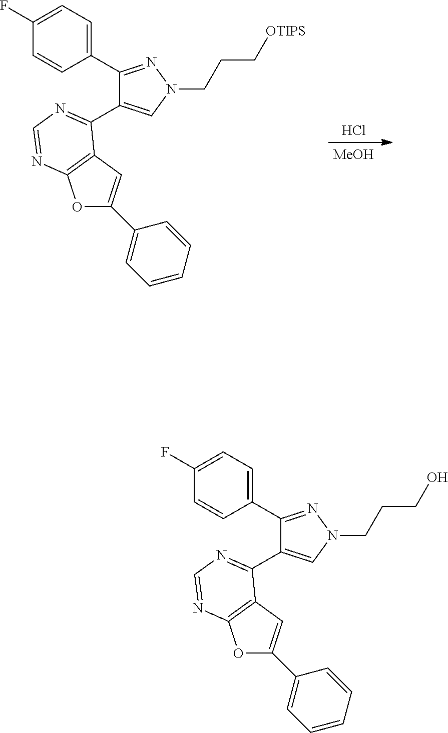

HCl (788 μL, 3.15 mmol, 4 M in dioxane, 40 equiv.) was added dropwise to a mixture of 4-(3-(4-fluorophenyl)-1-(3-((triisopropylsilyl)oxy)propyl)-1H-pyrazol-4-yl)-6-phenylfuro[2,3-d]pyrimidine (45.0 mg, 78.8 μmol) in MeOH (2.63 mL). The mixture was stirred at 25° C. for 1 hour. The reaction mixture was concentrated under reduced pressure. The residue was purified by reverse phase chromatography (C18 column, MeCN in water with 10 mM ammonium formate, 5-60%, a gradient elution) to provide the title compound (17.0 mg, 52%) as a solid. ¹H NMR (400 MHz, DMSO-d₆) δ 8.76 (s, 1H), 8.64 (s, 1H), 7.93 (d, J=7.9 Hz, 2H), 7.66-7.53 (m, 4H), 7.53-7.47 (m, 1H), 7.45 (d, J=1.3 Hz, 1H), 7.19 (t, J=8.0 Hz, 2H), 4.71 (t, J=4.4 Hz, 1H), 4.34 (t, J=6.9 Hz, 2H), 3.56-3.43 (m, 2H), 2.15-2.00 (m, 2H). m/z (ESI, +ve ion)=415.2 [M+H]⁺.

### Example 12. 3-[1-(4-fluorophenyl)-5-{2-phenyl-1H-pyrrolo[2,3-b]pyridin-4-yl}pyrazol-3-yl]propan-1-ol

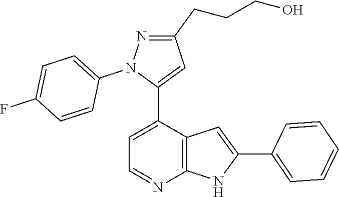 [SMILES: OCCCc1cc(-c2ccnc3[nH]c(-c4ccccc4)cc23)n(-c2ccc(F)cc2)n1]

### Step 1. 1-(Methoxymethyl)-2-phenyl-4-(4,4,5,5-tetramethyl-1,3,2-dioxaborolan-2-yl)pyrrolo[2,3-b]pyridine

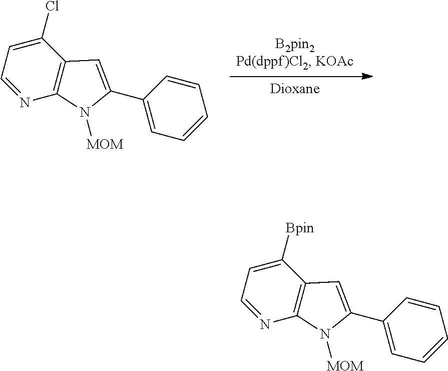

To a stirred mixture of 4-chloro-1-(methoxymethyl)-2-phenylpyrrolo[2,3-b]pyridine (Intermediate B, 100 mg, 0.367 mmol, 1.00 equiv) and bis(pinacolato)diboron (140 mg, 0.55 mmol, 1.5 equiv) in 1,4-dioxane (1 mL) were added Pd(dppf)Cl₂·CH₂Cl₂ (59.7 mg, 0.073 mmol, 0.2 equiv) and KOAc (108 mg, 1.10 mmol, 3 equiv) at room temperature under nitrogen atmosphere. The resulting mixture was stirred for 5 h at 80° C. under nitrogen atmosphere then cool down to room temperature. The resulting mixture was filtered, the filter cake was washed with EtOAc (3×30 mL). The filtrate was concentrated under reduced pressure to afford the title compound (200 mg, 99%) as a black oil. m/z (ESI+ve ion)=365.35 [M+H]⁺.

### Step 2. 4-[(tert-Butyldiphenylsilyl)oxy]butanoic acid

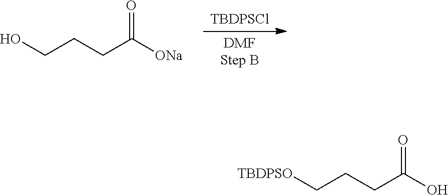

A mixture of sodium 4-hydroxybutanoate (4 g, 31.7 mmol, 1.00 equiv) and TBDPSCl (8.72 g, 31.7 mmol, 1 equiv) in DMF (40 mL) was stirred for overnight at room temperature under nitrogen atmosphere. The reaction was quenched by the addition of water (200 mL) at room temperature and extracted with EtOAc (3×150 mL). The combined organic layers were washed with brine (3×100 mL), dried over anhydrous Na₂SO₄. After filtration, the filtrate was concentrated under reduced pressure. The residue was purified by silica gel column chromatography, eluted with PE:EtOAc (1:2) to afford the title compound (7.6 g, 70%) as a colorless oil. m/z (ESI, +ve ion)=343.15 [M+H]⁺.

### Step 3. Methyl 4-[(tert-butyldiphenylsilyl)oxy]butanoate

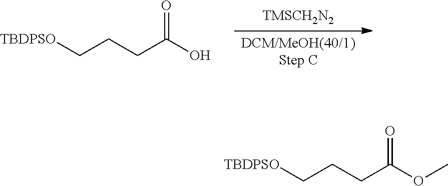

To a stirred mixture of 4-[(tert-butyldiphenylsilyl)oxy]butanoic acid (5 g, 14.6 mmol, 1.00 equiv) in DCM (40 mL) and MeOH (1 mL) was added TMSCHN₂ (20.4 mL, 40.9 mmol, 2.8 equiv) dropwise over 15 min at 0° C. under nitrogen atmosphere. The resulting mixture was stirred for 1 h at 0° C. under nitrogen atmosphere then warmed up to room temperature. The reaction was quenched by the addition of water (200 mL) at room temperature then extracted with EtOAc (3×150 mL). The combined organic layers were washed with brine (3×100 mL), dried over anhydrous Na₂SO₄. After filtration, the filtrate was concentrated under reduced pressure. The residue was purified by silica gel column chromatography, eluted with PE: EtOAc (1:1) to afford the title compound (4 g, 77%) as a yellow oil. m/z (ESI, +ve ion)=357.25 [M+H]⁺.

### Step 4. 6-[(tert-Butyldiphenylsilyl)oxy]-3-oxohexanenitrile

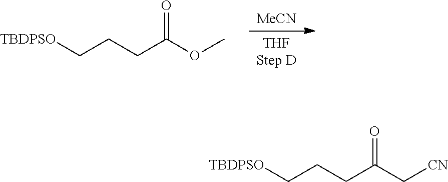

To a stirred mixture of MeCN (276 mg, 6.73 mmol, 1.2 equiv) in THF (20 mL) was added n-BuLi (2.69 mL, 6.73 mmol, 1.2 equiv, 2.5 M in hexane) dropwise over 20 min at −78° C. under nitrogen atmosphere. The resulting mixture was stirred for 2 h at −78° C. under nitrogen atmosphere. To the above mixture was added methyl 4-[(tert-butyldiphenylsilyl)oxy]butanoate (2 g, 5.61 mmol, 1.00 equiv) dropwise over 10 min at −78° C. under nitrogen atmosphere. The resulting mixture was stirred for additional 2 h at room temperature under nitrogen atmosphere. The reaction was quenched by the addition of water (200 mL) at room temperature. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with DCM:MeOH (20:1) to afford the title compound (1.5 g, 73%) as a colorless oil. m/z (ESI, +ve ion)=366.20 [M+H]⁺.

### Step 5. 5-{3-[(tert-Butyldiphenylsilyl)oxy]propyl}-2-(4-fluorophenyl)pyrazol-3-amine

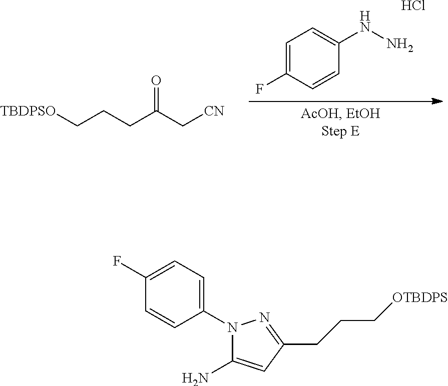

To a stirred mixture of 6-[(tert-butyldiphenylsilyl)oxy]-3-oxohexanenitrile (950 mg, 2.60 mmol, 1.00 equiv) and (4-fluorophenyl)hydrazine hydrochloride (465 mg, 2.86 mmol, 1.1 equiv) in EtOH (9 mL) was added AcOH (780 mg, 13.0 mmol, 5 equiv) dropwise at room temperature under nitrogen atmosphere. The resulting mixture was stirred for 1 h at 85° C. under nitrogen atmosphere then quenched by the addition of water (200 mL) at room temperature. The resulting mixture was extracted with EtOAc (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with DCM:MeOH (10:1) to afford the title compound (810 mg, 66%) as a colorless oil. m/z (ESI+ve ion)=474.35 [M+H]⁺.

### Step 6. 5-{3-[(tert-Butyldiphenylsilyl)oxy]propyl}-4-chloro-2-(4-fluorophenyl)pyrazol-3-amine

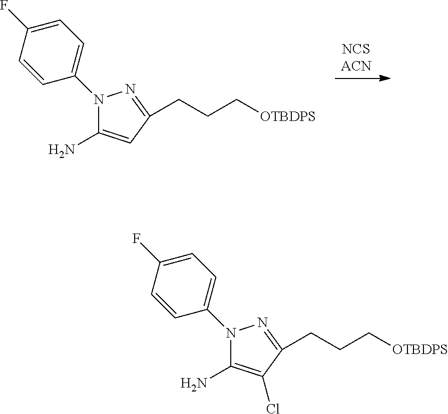

A mixture of 5-{3-[(tert-butyldiphenylsilyl)oxy]propyl}-2-(4-fluorophenyl)pyrazol-3-amine (500 mg, 1.06 mmol, 1.00 equiv) and NCS (141 mg, 1.06 mmol, 1 equiv) in ACN (5 mL) was stirred for 1 h at room temperature under nitrogen atmosphere. The reaction was quenched by the addition of water (100 mL) at room temperature then extracted with EtOAc (3×60 mL). The combined organic layers were washed with brine (2×50 mL), dried over anhydrous Na₂SO₄. After filtration, the filtrate was concentrated under reduced pressure. The residue was purified by silica gel column chromatography, eluted with PE/EtOAc (1/1) to afford the title compound (410 mg, 72%) as an orange oil. m/z (ESI, +ve ion)=508.30 [M+H]⁺.

### Step 7. 5-Bromo-3-{3-[(tert-butyldiphenylsilyl)oxy]propyl}-4-chloro-1-(4-fluorophenyl)pyrazole

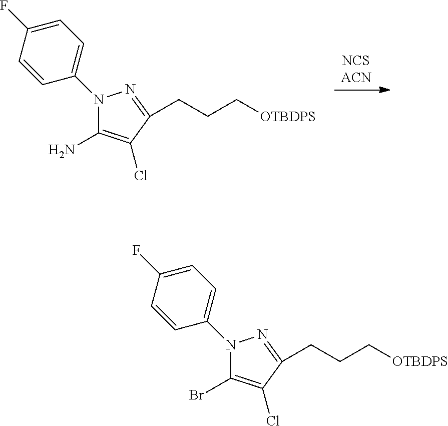

To a stirred mixture of 5-{3-[(tert-butyldiphenylsilyl)oxy]propyl}-4-chloro-2-(4-fluorophenyl)pyrazol-3-amine (500 mg, 0.98 mmol, 1.00 equiv) in ACN (5 mL) was added 3-methylbutyl nitrite (173 mg, 1.49 mmol, 1.5 equiv) dropwise at room temperature under nitrogen atmosphere. To the above mixture were added CuBr (179 mg, 1.18 mmol, 1.2 equiv) and CuBr₂ (264 mg, 1.18 mmol, 1.2 equiv) in portions at room temperature under nitrogen atmosphere. The resulting mixture was stirred for additional 1.5 h at room temperature under nitrogen atmosphere then quenched by the addition of water (100 mL) at room temperature. The resulting mixture was extracted with EtOAc (3×60 mL). The combined organic layers were washed with brine (2×50 mL), dried over anhydrous Na₂SO₄. After filtration, the filtrate was concentrated under reduced pressure. The residue was purified by reverse flash chromatography with the following conditions: Column: XBridge Prep Phenyl OBD Column, 19×150 mm, 5 μm; Mobile Phase A: water (10 mmol/L NH₄HCO₃), Mobile Phase B: ACN; Flow rate: 60 mL/min; Gradient: 80% B to 80% B in 8 min, 80% B; Wavelength: 254 nm; RT1 (min): 7 to afford the title compound (150 mg, 27%) as an orange oil. m/z (ESI+ve ion)=573.20 [M+H]⁺.

### Step 8. 3-{3-[(tert-Butyldiphenylsilyl)oxy]propyl}-4-chloro-1-(4-fluorophenyl)-5-[1-(methoxymethyl)-2-phenylpyrrolo[2,3-b]pyridin-4-yl]pyrazole

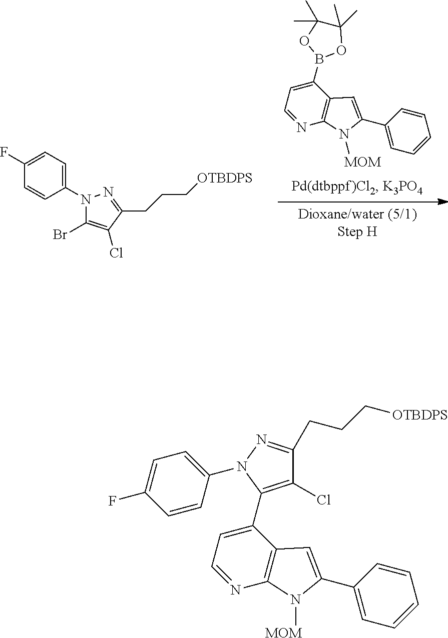

To a stirred mixture of 5-bromo-3-{3-[(tert-butyldiphenylsilyl)oxy]propyl}-4-chloro-1-(4-fluorophenyl)pyrazole (100 mg, 0.175 mmol, 1.00 equiv) and 1-(methoxymethyl)-2-phenyl-4-(4,4,5,5-tetramethyl-1,3,2-dioxaborolan-2-yl)pyrrolo[2,3-b]pyridine (96 mg, 0.262 mmol, 1.5 equiv) in 1,4-dioxane (1 mL) and H₂O (0.2 mL) were added Pd(DtBPF)Cl₂ (23 mg, 0.035 mmol, 0.2 equiv) and K₃PO₄ (74 mg, 0.350 mmol, 2 equiv) at room temperature under nitrogen atmosphere. The resulting mixture was stirred for 2 h at 90° C. under nitrogen atmosphere. The reaction was monitored by LCMS. The mixture was allowed to cool down to room temperature. The reaction was quenched by the addition of water (50 mL) at room temperature. The resulting mixture was extracted with EtOAc (3×30 mL). The combined organic layers were washed with brine (60 mL), dried over anhydrous Na₂SO₄. After filtration, the filtrate was concentrated under reduced pressure. The residue was purified by silica gel column chromatography, eluted with PE:EtOAc (1:1) to afford the title compound (100 mg, 78%) as an orange oil. m/z (ESI+ve ion)=729.40 [M+H]⁺.

### Step 9. 4-(3-(3-((tert-Butyldiphenylsilyl)oxy)propyl)-1-(4-fluorophenyl)-1H-pyrazol-5-yl)-2-phenyl-1H-pyrrolo[2,3-b]pyridine

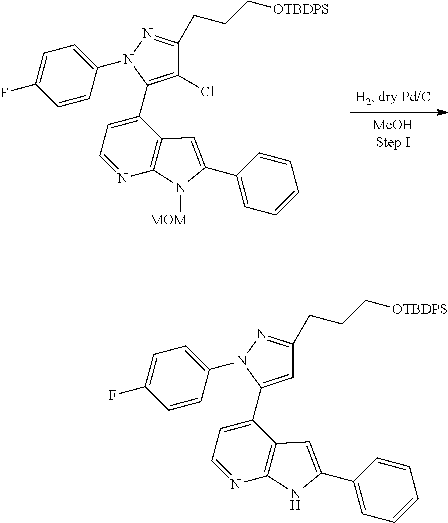

To a stirred mixture of 3-{3-[(tert-butyldiphenylsilyl)oxy]propyl}-4-chloro-1-(4-fluorophenyl)-5-[1-(methoxymethyl)-2-phenylpyrrolo[2,3-b]pyridin-4-yl]pyrazole (140 mg, 0.192 mmol, 1.00 equiv) in MeOH (4 mL) was added dry Pd/C (2.04 g, 1.92 mmol, 10 equiv, 10%) in portions at room temperature under nitrogen atmosphere. The mixture was hydrogenated for overnight at 60° C. under hydrogen atmosphere using a hydrogen balloon. The mixture was filtered through a Celite pad and concentrated under reduced pressure to afford the title compound (100 mg, 75) as a yellow oil. m/z (ESI+ve ion)=651.40 [M+H]⁺.

### Step 10. 3-[1-(4-Fluorophenyl)-5-{2-phenyl-1H-pyrrolo[2,3-b]pyridin-4-yl}pyrazol-3-yl]propan-1-ol

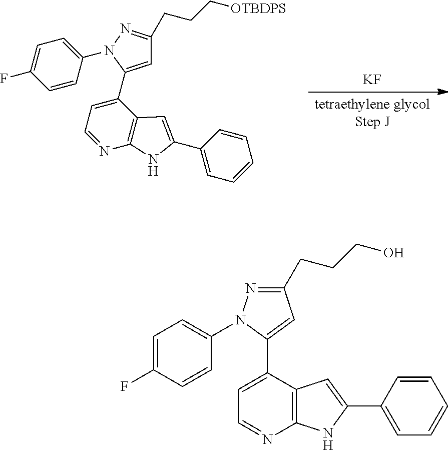

A mixture of 3-{3-[(tert-butyldiphenylsilyl)oxy]propyl}-1-(4-fluorophenyl)-5-[1-(methoxymethyl)-2-phenylpyrrolo[2,3-b]pyridin-4-yl]pyrazole (170 mg, 0.25 mmol, 1.00 equiv) and KF (71.06 mg, 1.22 mmol, 5 equiv) in tetraethylene glycol (5 mL) was stirred for overnight at 60° C. The mixture was cooled down to room temperature and quenched by the addition of water (50 mL) at room temperature. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by reverse flash chromatography with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 35% B to 45% B in 8 min, 45% B; Wavelength: 220 nm; RT1 (min): 6.7 to afford Example 12 (10.7 mg, 11%) as a white solid. m/z (ESI+ve ion)=413.20 [M+H]⁺. ¹H-NMR (400 MHz, Methanol-d₄) δ 8.12 (d, J=4.8 Hz, 1H), 7.79 (d, J=7.2 Hz, 2H), 7.49-7.45 (m, 2H), 7.39-7.32 (m, 3H), 7.14-7.09 (m, 2H), 6.85 (d, J=4.8 Hz, 1H), 6.75 (s, 1H), 6.65 (s, 1H), 3.74-3.70 (m, 2H), 2.90-2.86 (m, 2H), 2.07-2.01 (m, 2H).

### Example 13. 3-[3-(4-Fluorophenyl)-4-{2-[4-(4-methylpiperazin-1-yl)phenyl]-3H-imidazo[4,5-b]pyridin-7-yl}-1H-pyrazol-1-yl]propan-1-ol

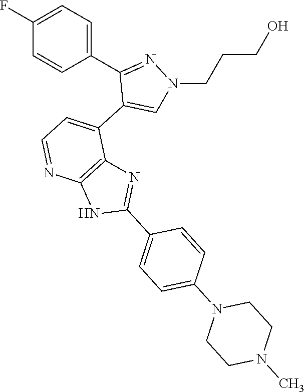 [SMILES: CN1CCN(c2ccc(-c3nc4c(-c5cn(CCCO)nc5-c5ccc(F)cc5)ccnc4[nH]3)cc2)CC1]

### Step 1. 7-Bromo-2-(4-(4-methylpiperazin-1-yl)phenyl)-3H-imidazo[4,5-b]pyridine

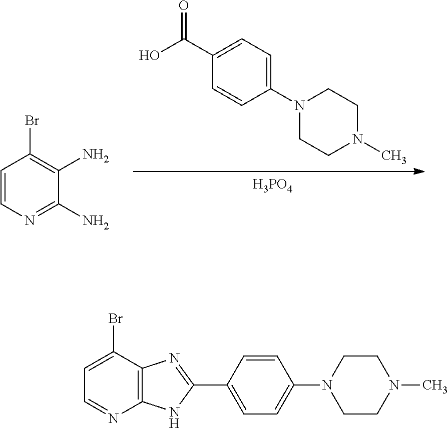

4-Bromopyridine-2,3-diamine (170 mg, 859 μmol) and 4-(4-methylpiperazin-1-yl)benzoic acid (208 mg, 945 μmol, 1.1 equiv.) was suspended in polyphosphoric acid (10.0 g). The mixture was stirred for 16 hours at 130° C., then cooled to room temperature. Ice was added and the reaction mixture was triturated. This process was repeated until the mixture became fluid and precipitate formation was observed. The resulting mixture was vigorously stirred for 30 minutes and resulting solid were collected by vacuum filtration, while washing with water, to provide the title compound (268 mg, 84%). m/z (ESI, +ve ion)=372.2, 374.0 [M+H]⁺.

### Step 2. 7-Iodo-2-(4-(4-methylpiperazin-1-yl)phenyl)-3H-imidazo[4,5-b]pyridine

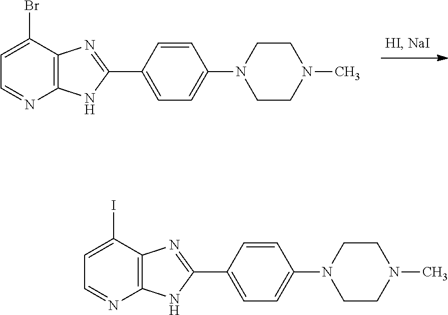

Hydriodic acid (1.51 mL, 6.63 mmol, 25 equiv.) was added to 7-bromo-2-(4-(4-methylpiperazin-1-yl)phenyl)-3H-imidazo[4,5-b]pyridine (87.0 mg, 265 μmol) at 0° C. and the mixture was stirred for 3 h at 0° C. Sodium iodide (121 mg, 796 μmol) was added and the mixture was heated to 60° C. for 4 h. The reaction mixture was diluted with ice/water mixture (5 mL). The mixture was stirred vigorously until all of the ice had melted. 15% w/w aq. NaOH solution was added until a pH of 6-7 was obtained. EtOAc (20 mL) and water (10 mL) were added, and the layers were separated. The aqueous layer was extracted with additional EtOAc (3×20 mL) and the combined organic layers were washed with water (10 mL), brine (10 mL), dried over Na₂SO₄, filtered, and concentrated under reduced pressure to provide the title compound (55.0 mg, 49%) as a solid. m/z (ESI, +ve ion)=420.1 [M+H]⁺.

### Step 3. 7-(3-(4-Fluorophenyl)-1-(3-((triisopropylsilyl)oxy)propyl)-1H-pyrazol-4-yl)-2-(4-(4-methylpiperazin-1-yl)phenyl)-3H-imidazo[4,5-b]pyridine

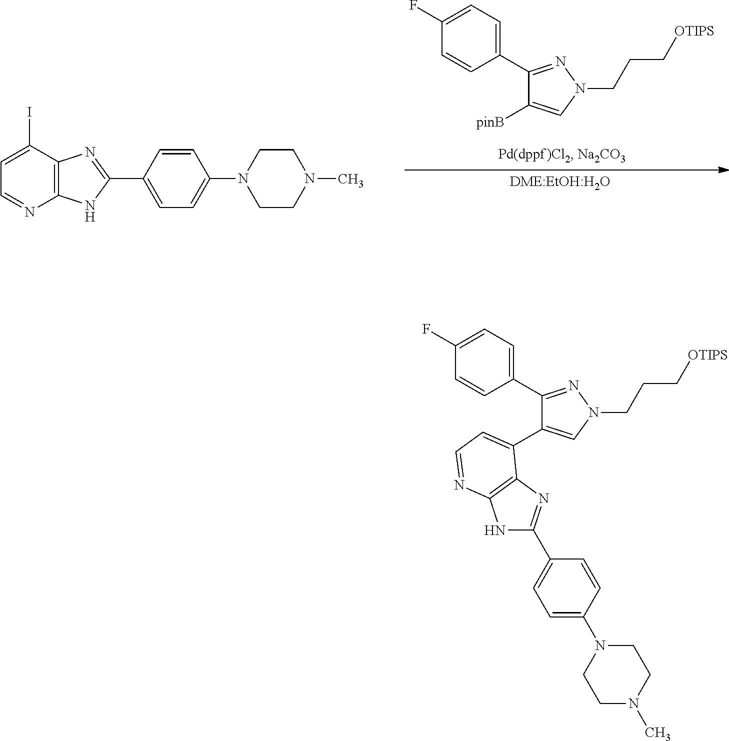

1,1′-Bis(diphenylphosphino)ferrocene dichloropalladium (II) (20.1 mg, 26.9 μmol, 0.1 equiv.) was added to a degassed mixture of 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 135 mg, 269 μmol, 1.0 equiv.), 7-iodo-2-(4-(4-methylpiperazin-1-yl)phenyl)-3H-imidazo[4,5-b]pyridine (100 mg, 269 μmol) and sodium carbonate (86.3 mg, 806 μmol) in a mixture of DME (1.40 mL), EtOH (790 μL) and H₂O (345 μL). The mixture was stirred for 30 minutes at 110° C. in a microwave reactor. The reaction was further heated at 110° C. for 80 hours. The mixture was cooled and concentrated under reduced pressure. The residue was purified by column chromatography (dry loading, MeOH in DCM, 0-20%, a gradient elution), to provide the title compound (158 mg, 75%) as a solid. m/z (ESI, +ve ion)=668.6 [M+H]⁺.

### Step 4. 3-[3-(4-Fluorophenyl)-4-{2-[4-(4-methylpiperazin-1-yl)phenyl]-3H-imidazo[4,5-b]pyridin-7-yl}-1H-pyrazol-1-yl]propan-1-ol

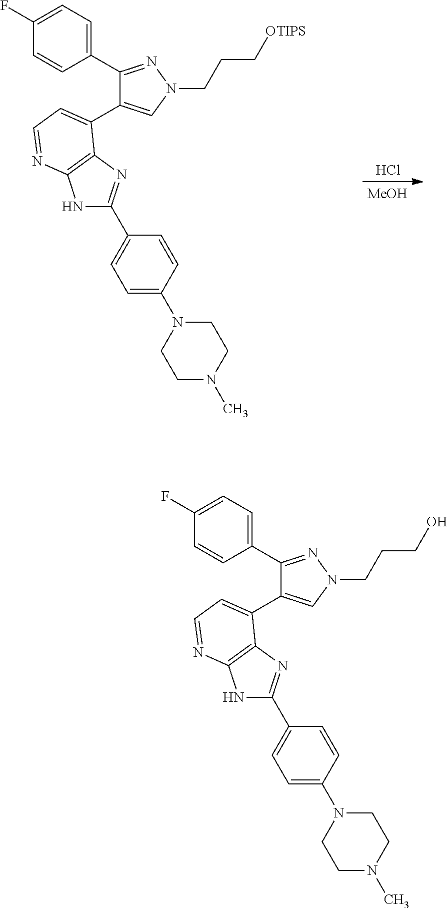

HCl (1.54 mL, 6.15 mmol, 4 M in dioxane, 40 equiv.) was added dropwise to a mixture of 7-(3-(4-fluorophenyl)-1-(3-((triisopropylsilyl)oxy)propyl)-1H-pyrazol-4-yl)-2-(4-(4-methylpiperazin-1-yl)phenyl)-3H-imidazo[4,5-b]pyridine (158 mg, 154 μmol) in MeOH (5.15 mL). The mixture was stirred at 25° C. for 1 hour. The reaction mixture was concentrated under reduced pressure. The residue was purified by reverse phase chromatography (C18 column, MeCN in water with 10 mM ammonium formate, 5-60%, a gradient elution) to provide the title compound (17.0 mg, 22%) as a solid. ¹H NMR (400 MHz, DMSO-d₆) δ 8.64 (s, 1H), 8.07 (d, J=8.9 Hz, 2H), 8.02 (d, J=5.2 Hz, 1H), 7.55-7.45 (m, 2H), 7.21 (t, J=8.9 Hz, 2H), 7.07 (d, J=9.0 Hz, 2H), 6.81 (d, J=5.2 Hz, 1H), 4.69 (t, J=5.1 Hz, 1H), 4.33 (t, J=7.1 Hz, 2H), 3.57-3.46 (m, 2H), 3.30-3.24 (m, 4H), 2.48-2.41 (m, 4H), 2.23 (s, 3H), 2.11-1.98 (m, 2H). m/z (ESI, +ve ion)=512.4 [M+H]⁺.

### Example 14. 3-{3-(4-Fluorophenyl)-4-[2-(1-methyl-1,2,3,6-tetrahydropyridin-4-yl)-1H-pyrrolo[2,3-b]pyridin-4-yl]-1H-pyrazol-1-yl}propan-1-ol

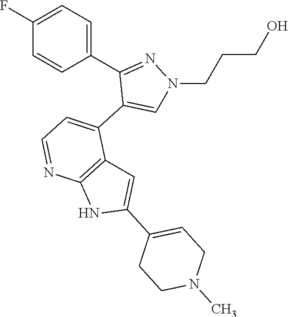 [SMILES: CN1CC=C(c2cc3c(-c4cn(CCCO)nc4-c4ccc(F)cc4)ccnc3[nH]2)CC1]

### Step 1. 2-Bromo-4-(3-(4-fluorophenyl)-1-(3-((triisopropylsilyl)oxy)propyl)-1H-pyrazol-4-yl)-1-(phenylsulfonyl)-1H-pyrrolo[2,3-b]pyridine

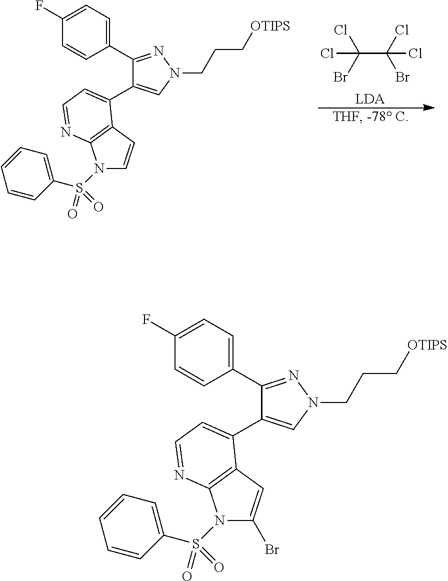

Lithium diisopropylamide (180 μL, 360 μmol, 1.2 equiv.) was added to a mixture of 4-(3-(4-fluorophenyl)-1-(3-((triisopropylsilyl)oxy)propyl)-1H-pyrazol-4-yl)-1-(phenylsulfonyl)-1H-pyrrolo[2,3-b]pyridine (Intermediate J, 190 mg, 300 μmol) in THF (1.34 mL) at −78° C. The mixture was stirred for 1 hour at −78° C. then 1,2-dibromotetrachloroethane (121 mg, 360 μmol, 1.2 equiv.) in THF (267 μL) was added. The mixture was stirred for 30 minutes at −78° C. then slowly warmed to 0° C. The reaction mixture was diluted EtOAc (3 mL) followed by water (5 mL) and vigorously stirred for 3 minutes. The layers were separated, and aqueous layer was extracted with additional EtOAc (3×4 mL). The combined organic layers were washed with brine, then dried over Na₂SO₄, filtered, and concentrated under reduced pressure. The residue was purified by column chromatography (DCM in hexanes, 0-20%, a gradient elution) to provide the title compound (190 mg, 89%) as an oil. m/z (ESI, +ve ion)=711.3, 713.2 [M+H]⁺.

### Step 2. 4-(3-(4-Fluorophenyl)-1-(3-((triisopropylsilyl)oxy)propyl)-1H-pyrazol-4-yl)-2-(1-methyl-1,2,3,6-tetrahydropyridin-4-yl)-1-(phenylsulfonyl)-1H-pyrrolo[2,3-b]pyridine

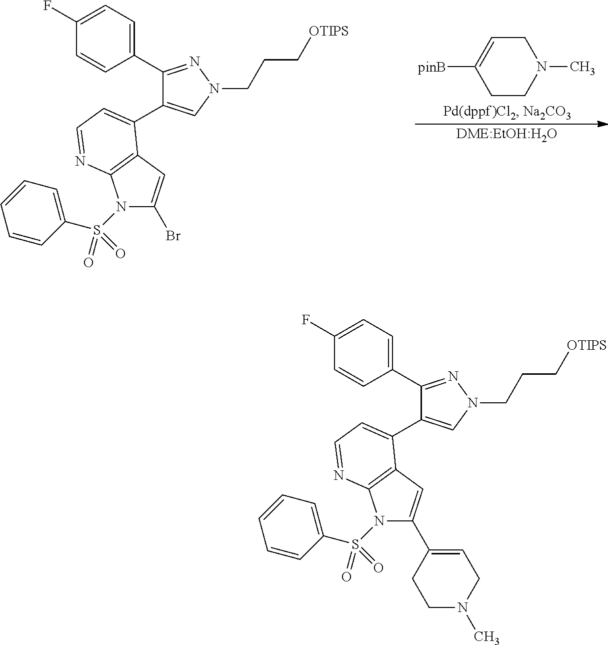

1,1′-Bis(diphenylphosphino)ferrocene dichloropalladium (II) (11.9 mg, 15.5 μmol, 0.05 equiv.) was added to a degassed mixture of 1-methyl-1,2,3,6-tetrahydropyridine-4-boronic acid pinacol ester (75.9 mg, 340 μmol, 1.1 equiv.), sodium carbonate (99.3 mg, 927 μmol, 3.0 equiv.) and 2-bromo-4-(3-(4-fluorophenyl)-1-(3-((triisopropylsilyl)oxy)propyl)-1H-pyrazol-4-yl)-1-(phenylsulfonyl)-1H-pyrrolo[2,3-b]pyridine (220 mg, 309 μmol) in a mixture of DME (1.54 mL), EtOH (890 μL) and H₂O (385 μL). The mixture was stirred for 16 hours at 110° C. in the oil bath. The mixture was diluted with water (30 mL) and extracted with EtOAc (3×20 mL). The combined organic layers were washed with brine, then dried over Na₂SO₄, filtered, and concentrated under reduced pressure. The residue was purified by column chromatography (MeOH in DCM, 0-20%, a gradient elution) to provide the title compound (180 mg, 80%) as an oil. m/z (ESI, +ve ion)=728.5 [M]⁺.

### Step 3. 4-(3-(4-Fluorophenyl)-1-(3-((triisopropylsilyl)oxy)propyl)-1H-pyrazol-4-yl)-2-(1-methyl-1,2,3,6-tetrahydropyridin-4-yl)-1-(phenylsulfonyl)-1H-pyrrolo[2,3-b]pyridine

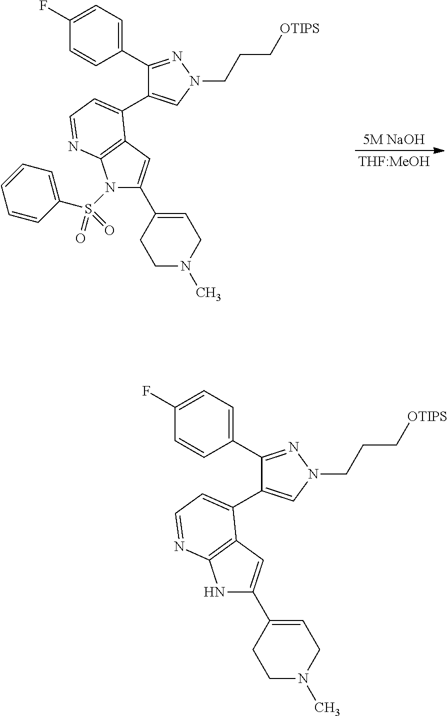

Sodium hydroxide (113 μL, 566 μmol, 5 M in water, 4.0 equiv.) was added to a solution of 4-(3-(4-fluorophenyl)-1-(3-((triisopropylsilyl)oxy)propyl)-1H-pyrazol-4-yl)-2-(1-methyl-1,2,3,6-tetrahydropyridin-4-yl)-1-(phenylsulfonyl)-1H-pyrrolo[2,3-b]pyridine (101 mg, 139 μmol) in a mixture of THF (750 μL) and MeOH (750 μL). The mixture was stirred for 1 hour at 70° C. then cooled to room temperature and diluted with a saturated solution of aqueous NH₄Cl (5 mL), water (2 mL) and extracted with EtOAc (3×5 mL). The combined organic layers were washed with water, then dried over Na₂SO₄, filtered, and concentrated to provide the title compound (72.5 mg, 90%) as a solid. m/z (ESI, +ve ion)=588.6 [M+H]⁺.

### Step 4. 3-[3-(4-Fluorophenyl)-4-{2-[4-(4-methylpiperazin-1-yl)phenyl]-3H-imidazo[4,5-b]pyridin-7-yl}-1H-pyrazol-1-yl]propan-1-ol

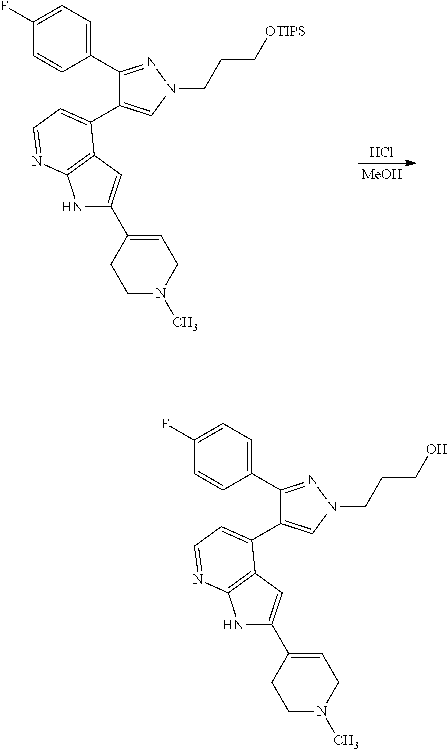

HCl (1.05 mL, 4.18 mmol, 4 M in dioxane, 30 equiv.) was added dropwise to a mixture of 4-(3-(4-fluorophenyl)-1-(3-((triisopropylsilyl)oxy)propyl)-1H-pyrazol-4-yl)-2-(1-methyl-1,2,3,6-tetrahydropyridin-4-yl)-1-(phenylsulfonyl)-1H-pyrrolo[2,3-b]pyridine (82.0 mg, 139 μmol) in MeOH (4.68 mL). The reaction mixture was stirred at room temperature for 1 hour, then concentrated under reduced pressure. The residue was purified by reverse phase chromatography (MeCN in 10 mM ammonium bicarbonate in water, 5-60%, a gradient elution) to provide the title compound (13.0 mg, 22%) as a solid. ¹H NMR (400 MHz, DMSO-d₆) δ 11.72 (s, 1H), 8.18 (s, 1H), 8.04 (d, J=5.0 Hz, 1H), 7.42-7.35 (m, 2H), 7.18-7.09 (m, 2H), 6.74 (d, J=5.0 Hz, 1H), 6.43 (t, J=3.1 Hz, 1H), 6.12 (d, J=1.8 Hz, 1H), 4.67 (t, J=5.1 Hz, 1H), 4.28 (t, J=7.1 Hz, 2H), 3.49 (dd, J=11.1, 5.9 Hz, 2H), 3.07-2.99 (m, 2H), 2.52 (d, J=5.8 Hz, 2H), 2.41-2.32 (m, 2H), 2.27 (s, 3H), 2.09-1.98 (m, 2H). m/z (ESI, +ve ion)=432.3 [M+H]⁺.

### Example 15. 3-[3-(4-Fluorophenyl)-4-{8-[4-(4-methylpiperazin-1-yl)phenyl]-9H-purin-6-yl}-1H-pyrazol-1-yl]propan-1-ol

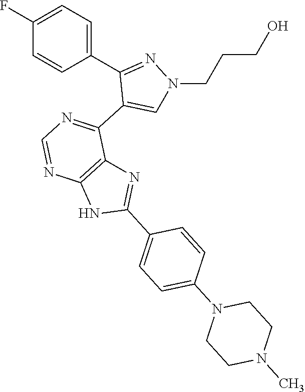 [SMILES: CN1CCN(c2ccc(-c3nc4c(-c5cn(CCCO)nc5-c5ccc(F)cc5)ncnc4[nH]3)cc2)CC1]

### Step 1. 6-Chloro-8-(4-(4-methylpiperazin-1-yl)phenyl)-9H-purine

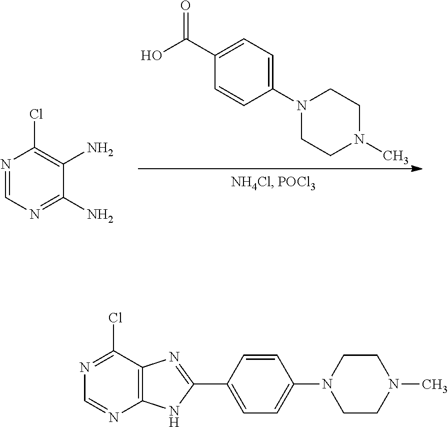 [SMILES: Nc1ncnc(Cl)c1N ; CN1CCN(c2ccc(C(=O)O)cc2)CC1 ; CN1CCN(c2ccc(-c3nc4c(Cl)ncnc4[nH]3)cc2)CC1]

Phosphorus oxychloride (3.03 mL, 32.2 mmol, 63 equiv.) was added to a mixture of 6-chloropyrimidine-4,5-diamine (75.7 mg, 508 μmol), 4-(4-methylpiperazin-1-yl)benzoic acid (113 mg, 513 μmol, 1.01 equiv.) and ammonium chloride (163 mg, 3.05 mmol, 6 equiv.). The mixture was stirred for 15 h at 100° C. then poured into an ice/water mixture. The pH was adjusted to 7-8 using NH₄OH (28-30% NH₃ in water). The aqueous phase was extracted with CHCl₃:IPA (9:1 v/v) (5×20 mL). The combined organic layers were dried over Na₂SO₄, filtered, and concentrated under reduced pressure to provide the title compound (109 mg, 65%) as a solid. m/z (ESI, +ve ion)=329.2, 331.1 [M+H]⁺.

### Step 2. 6-Iodo-8-(4-(4-methylpiperazin-1-yl)phenyl)-9H-purine

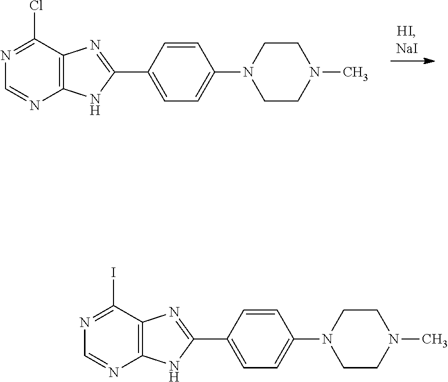 [SMILES: CN1CCN(c2ccc(-c3nc4c(Cl)ncnc4[nH]3)cc2)CC1 ; CN1CCN(c2ccc(-c3nc4c(I)ncnc4[nH]3)cc2)CC1]

6-Chloro-8-(4-(4-methylpiperazin-1-yl)phenyl)-9H-purine (256 mg, 506 μmol) was suspended in hydriodic acid (2.30 mL, 10.1 mmol, 20 equiv.) and the resulting mixture stirred at 0° C. for 10 minutes then diluted water. The pH of the aqueous layer was adjusted to 7-8 using ammonium hydroxide solution (28-30% NH₃ in water). The mixture was extracted with chloroform:IPA (9:1, v/v) (4×60 mL). The combined organic layers were washed with brine, dried over Na₂SO₄, filtered and concentrated. The residue was triturated in DCM and the precipitate was collect by suction filtration to provide the title compound (158 mg, 74%) as a solid. m/z (ESI, +ve ion)=421.1 [M+H]⁺.

### Step 3. 6-(3-(4-Fluorophenyl)-1-(3-((triisopropylsilyl)oxy)propyl)-1H-pyrazol-4-yl)-8-(4-(4-methylpiperazin-1-yl)phenyl)-9H-purine

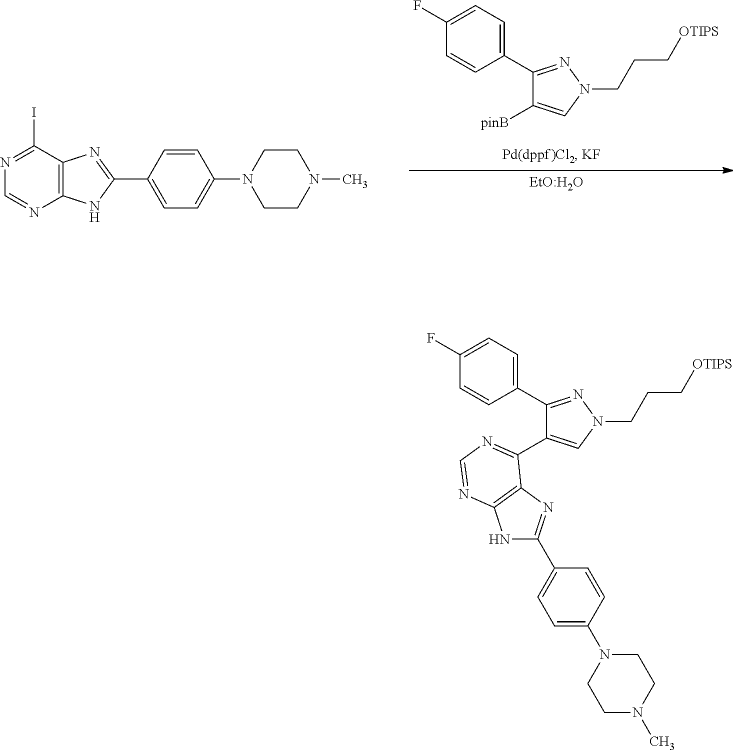

1,1′-Bis(diphenylphosphino)ferrocene dichloropalladium (II) (4.85 mg, 6.63 μmol, 0.1 equiv.) was added to a degassed mixture of potassium fluoride (7.78 mg, 133 μmol, 2.0 equiv.), 6-chloro-8-(4-(4-methylpiperazin-1-yl)phenyl)-9H-purine (27.9 mg, 66.3 μmol) and 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 40.0 mg, 79.6 μmol, 1.2 equiv.) in a mixture of EtOH (500 μL) and water (120 μL). The mixture was stirred for 10 min at 140° C. in a microwave reactor. Silica gel was added to the mixture and concentrated under reduced pressure. The dry-loaded residue was purified by column chromatography (MeOH in DCM, 0-10%, a gradient elution) to provide the title compound (22.0 mg, 50%) as a solid. m/z (ESI, +ve ion)=669.5 [M+H]⁺.

### Step 4. 3-[3-(4-Fluorophenyl)-4-{8-[4-(4-methylpiperazin-1-yl)phenyl]-9H-purin-6-yl}-1H-pyrazol-1-yl]propan-1-ol

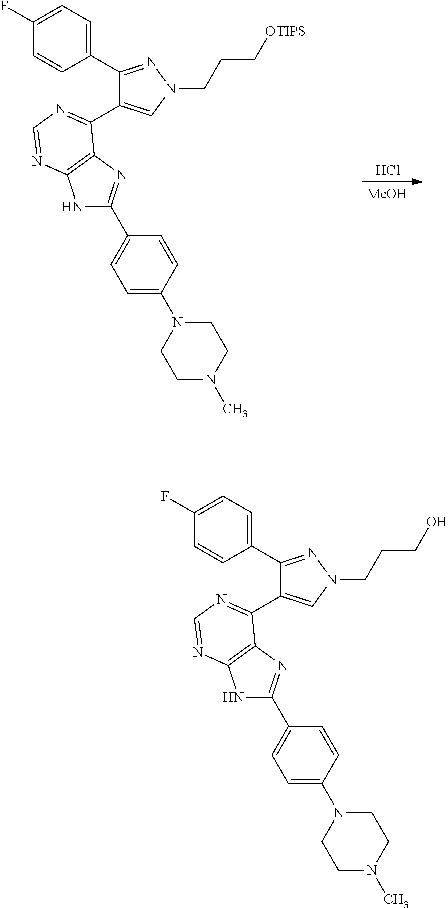

HCl (822 μL, 3.29 mmol, 4 M in dioxane, 40 equiv.) was added dropwise to a mixture of 6-(3-(4-fluorophenyl)-1-(3-((triisopropylsilyl)oxy)propyl)-1H-pyrazol-4-yl)-8-(4-(4-methylpiperazin-1-yl)phenyl)-9H-purine (55.0 mg, 82.2 μmol) in MeOH (2.76 mL). The mixture was stirred at room temperature for 15 min, then 15% w/w aq. NaOH solution in water was added dropwise until pH was adjusted to 7-8. The aqueous layer was extracted with EtOAc (3×10 mL). The combined organic layers were washed with water, dried over Na₂SO₄, filtered, and concentrated under reduced pressure. The residue was purified by reverse phase chromatography (MeCN in 10 mM ammonium formate in water, 10-100%, a gradient elution) to provide the title compound (25.0 mg, 59%) as a solid. ¹H NMR (400 MHz, DMSO-d₆) δ 8.93 (br s, 1H), 8.57 (s, 1H), 8.11 (d, J=9.0 Hz, 2H), 7.73-7.65 (m, 2H), 7.17 (t, J=8.9 Hz, 2H), 7.09 (d, J=9.0 Hz, 2H), 4.69 (br s, 1H), 4.36 (t, J=7.0 Hz, 2H), 3.51 (t, J=5.9 Hz, 2H), 3.30 (s, 4H), 2.48-2.41 (m, 4H), 2.23 (s, 3H), 2.06 (p, J=6.3 Hz, 2H). m/z (ESI, +ve ion)=513.3 [M+H]⁺.

### Example 16. 4-(4-Fluorophenyl)-1-(3-hydroxypropyl)-3-(2-phenyl-1H-pyrrolo[2,3-b]pyridin-4-yl)-1H-pyrazole-5-carbonitrile

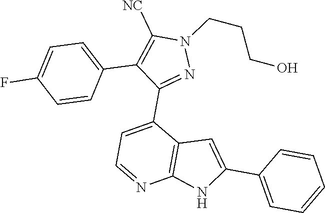 [SMILES: N#Cc1c(-c2ccc(F)cc2)c(-c2ccnc3[nH]c(-c4ccccc4)cc23)nn1CCCO]

### Step 1. Ethyl (2E)-2-cyano-3-(4-fluorophenyl)prop-2-enoate

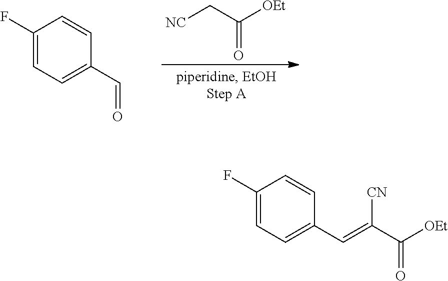 [SMILES: O=Cc1ccc(F)cc1 ; CCOC(=O)CC#N ; CCOC(=O)/C(C#N)=C/c1ccc(F)cc1]

To a stirred mixture of 4-fluorobenzaldehyde (10 g, 80.6 mmol, 1.00 equiv) and ethyl cyanoacetate (9.57 g, 84.6 mmol, 1.05 equiv) in EtOH (40 mL, 689 mmol, 8.55 equiv) was added piperidine (0.14 g, 1.61 mmol, 0.02 equiv) dropwise at room temperature under nitrogen atmosphere. The resulting mixture was stirred for 16 h at 85° C. under nitrogen atmosphere then cooled down to room temperature. The resulting mixture was filtered, the filter cake was washed with EtOH (3×10 mL). This resulted in the title compound (11.8 g, 67%) as a yellow solid. m/z (ESI, +ve ion)=220.10 [M+H]⁺.

### Step 2. Ethyl 4-(4-fluorophenyl)-5-(trimethylsilyl)-1H-pyrazole-3-carboxylate

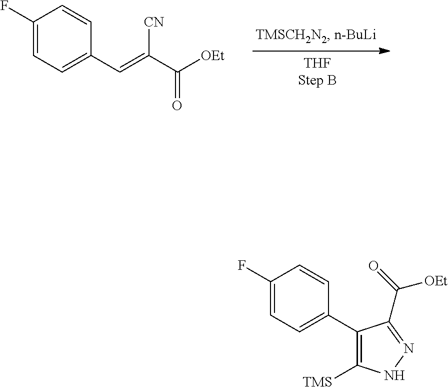 [SMILES: CCOC(=O)/C(C#N)=C/c1ccc(F)cc1 ; CCOC(=O)c1n[nH]c([Si](C)(C)C)c1-c1ccc(F)cc1]

To a stirred mixture of TMSCHN₂ (10.1 g, 88.6 mmol, 1.5 equiv) in dry THF (60 mL) were added n-BuLi (35.4 mL, 88.6 mmol, 1.5 equiv, 2.5 M in hexane) dropwise at −78° C. under nitrogen atmosphere. The resulting mixture was stirred for 30 min at −78° C. under nitrogen atmosphere. To the above mixture was added ethyl (2E)-2-cyano-3-(4-fluorophenyl)prop-2-enoate (12.95 g, 59.1 mmol, 1.00 equiv) in dry THF (60 mL) dropwise over 5 min at −78° C. The resulting mixture was stirred for additional 1 hour at −78° C. then at room temperature for 16 h. The reaction was quenched by the addition of sat. Na₂CO₃ (aq., 40 mL) at room temperature and extracted with EtOAc (3×30 mL). The combined organic layers were washed with brine (2×30 mL), dried over anhydrous Na₂SO₄. After filtration, the filtrate was concentrated under reduced pressure. The residue was purified by reverse phase flash chromatography with the following conditions: Column: Spherical C18, 20-40 μm, 330 g; Mobile Phase A: water (plus 0.05% TFA); Mobile Phase B: ACN; Flow rate: 100 mL/min; Gradient: 40% B-60% B in 30 min; Detector: 254 nm. The fractions containing desired product were collected at 51% B and concentrated under reduced pressure to afford the title compound (4.26 g, 23%) as a yellow solid. m/z (ESI, +ve ion)=307.15 [M+H]⁺.

### Step 3. Ethyl 5-bromo-4-(4-fluorophenyl)-1H-pyrazole-3-carboxylate

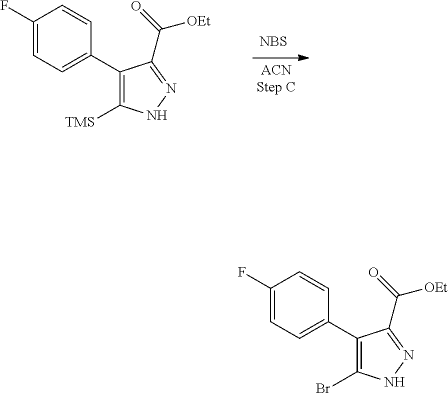

A mixture of ethyl 4-(4-fluorophenyl)-5-(trimethylsilyl)-1H-pyrazole-3-carboxylate (4.26 g, 13.9 mmol, 1.00 equiv) and NBS (2.72 g, 15.3 mmol, 1.1 equiv) in ACN (120 mL) was stirred for 16 h at room temperature under nitrogen atmosphere. The resulting mixture was concentrated under vacuum. The residue was purified by silica gel column chromatography, eluted with PE:EtOAc (2:1) to afford the title compound (3.9 g, 90%) as an off-white solid. m/z (ESI, +ve ion)=313.00, 315.00 [M+H]⁺.

### Step 4. Ethyl 5-bromo-2-{3-[(tert-butyldimethylsilyl)oxy]propyl}-4-(4-fluorophenyl)pyrazole-3-carboxylate

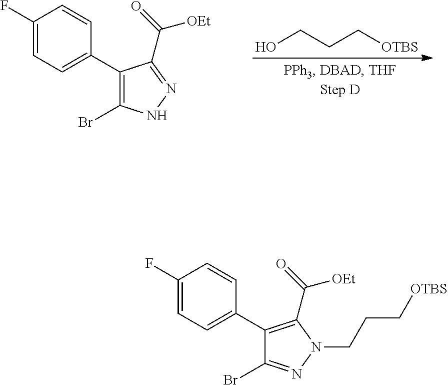 [SMILES: CCOC(=O)c1n[nH]c(Br)c1-c1ccc(F)cc1 ; CC(C)(C)[Si](C)(C)OCCCO ; CCOC(=O)c1c(-c2ccc(F)cc2)c(Br)nn1CCCO[Si](C)(C)C(C)(C)C]

To a stirred mixture of ethyl 5-bromo-4-(4-fluorophenyl)-1H-pyrazole-3-carboxylate (3.9 g, 12.5 mmol, 1.00 equiv) and 3-[(tert-butyldimethylsilyl)oxy]propan-1-ol (3.56 g, 18.7 mmol, 1.5 equiv) in THF (118 mL) were added PPh₃ (4.90 g, 18.7 mmol, 1.5 equiv) and DBAD (4.30 g, 18.7 mmol, 1.5 equiv) at room temperature under nitrogen atmosphere. The resulting mixture was stirred for 16 h at room temperature under nitrogen atmosphere then concentrated under vacuum. The residue was purified by reverse phase flash chromatography with the following conditions: Column: Spherical C18, 20-40 μm, 330 g; Mobile Phase A: water (plus 0.05% TFA); Mobile Phase B: ACN; Flow rate: 80 mL/min; Gradient: 70% B-90% B in 30 min; Detector: 254 nm. The fractions containing desired product were collected at 88% B and concentrated under reduced pressure to afford the title compound (1.3 g, 22%) as a colorless oil. m/z (ESI, +ve ion)=485.25, 487.25 [M+H]⁺.

### Step 5. 1-(Benzenesulfonyl)-2-phenyl-4-(4,4,5,5-tetramethyl-1,3,2-dioxaborolan-2-yl)pyrrolo[2,3-b]pyridine

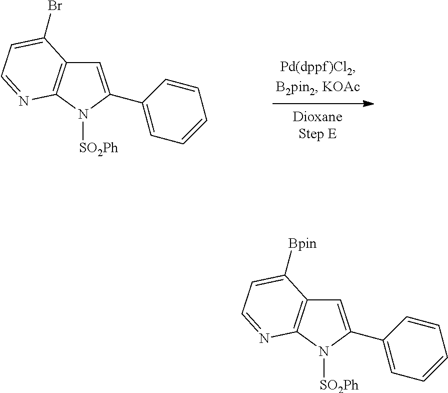

To a stirred mixture of 1-(benzenesulfonyl)-4-bromo-2-phenylpyrrolo[2,3-b]pyridine (Intermediate E, 100 mg, 0.24 mmol, 1.00 equiv) and 4,4,5,5-tetramethyl-2-(tetramethyl-1,3,2-dioxaborolan-2-yl)-1,3,2-dioxaborolane (92 mg, 0.36 mmol, 1.5 equiv) in dioxane (2 mL) were added KOAc (71 mg, 0.73 mmol, 3 equiv) and Pd(dppf)Cl₂·CH₂Cl₂ (39 mg, 0.048 mmol, 0.2 equiv) at room temperature under nitrogen atmosphere. The resulting mixture was stirred for 16 h at 80° C. under nitrogen atmosphere. The resulting mixture was filtered, the filter cake was washed with EtOAc (3×5 mL). The filtrate was concentrated under reduced pressure to afford the title compound (110 mg, 99%) as a black solid. m/z (ESI, +ve ion)=379.15 [M+H]⁺. MS was signal of boronic acid.

### Step 6. Ethyl 5-[1-(benzenesulfonyl)-2-phenylpyrrolo[2,3-b]pyridin-4-yl]-2-{3-[(tert-butyldimethylsilyl)oxy]propyl}-4-(4-fluorophenyl)pyrazole-3-carboxylate

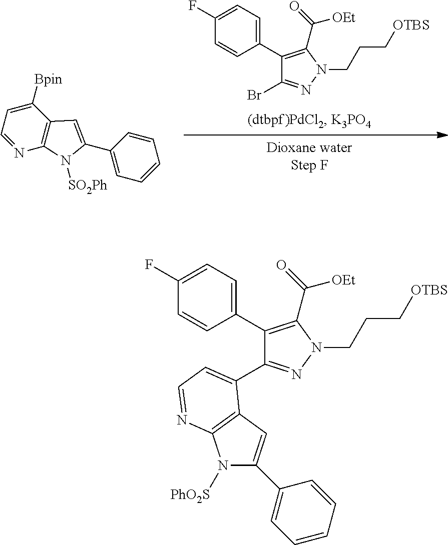

To a stirred mixture of 1-(benzenesulfonyl)-2-phenyl-4-(4,4,5,5-tetramethyl-1,3,2-dioxaborolan-2-yl)pyrrolo[2,3-b]pyridine (754 mg, 1.64 mmol, 1.5 equiv) and ethyl 5-bromo-2-{3-[(tert-butyldimethylsilyl)oxy]propyl}-4-(4-fluorophenyl)pyrazole-3-carboxylate (530 mg, 1.09 mmol, 1.00 equiv) in dioxane (10 mL) and H₂O (2 mL) were added Pd(DtBPF)Cl₂ (142 mg, 0.22 mmol, 0.2 equiv) and K₃PO₄ (464 mg, 2.18 mmol, 2 equiv) at room temperature under nitrogen atmosphere. The resulting mixture was stirred for 2 h at 90° C. under nitrogen atmosphere then cooled down to room temperature. The resulting mixture was diluted with water (10 mL) and extracted with EtOAc (3×15 mL). The combined organic layers were washed with brine (3×20 mL), dried over anhydrous Na₂SO₄. After filtration, the filtrate was concentrated under reduced pressure. The residue was purified by silica gel column chromatography, eluted with PE:EtOAc (1:1) to afford the title compound (630 mg, 78%) as an off-white solid. m/z (ESI, +ve ion)=739.35 [M+H]⁺.

### Step 7. {5-[1-(Benzenesulfonyl)-2-phenylpyrrolo[2,3-b]pyridin-4-yl]-2-{3-[(tert-butyldimethylsilyl)oxy]propyl}-4-(4-fluorophenyl)pyrazol-3-yl}methanol

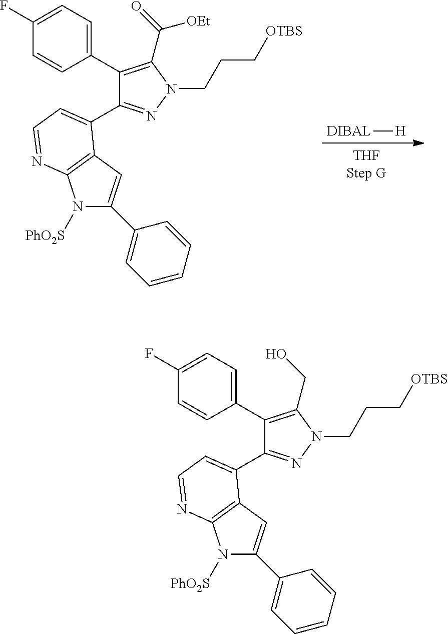

To a stirred mixture of ethyl 5-[1-(benzenesulfonyl)-2-phenylpyrrolo[2,3-b]pyridin-4-yl]-2-{3-[(tert-butyldimethylsilyl)oxy]propyl}-4-(4-fluorophenyl)pyrazole-3-carboxylate (630 mg, 0.85 mmol, 1 equiv) in THF (12 mL) was added DIBAL-H (4.3 mL, 4.27 mmol, 5 equiv, 1 M in toluene) dropwise at −78° C. under nitrogen atmosphere. The resulting mixture was stirred for 5 min at −78° C. under nitrogen atmosphere then for 16 h at room temperature. The resulting mixture was concentrated under reduced pressure and the residue was diluted with water (10 mL) and then extracted with EtOAc (3×15 mL). The combined organic layers were washed with brine (3×20 mL), dried over anhydrous Na₂SO₄. After filtration, the filtrate was concentrated under reduced pressure. The residue was purified by silica gel column chromatography, eluted with PE/EtOAc (1/1) to afford the title compound (320 mg, 54%) as an off-white solid. m/z (ESI, +ve ion)=697.30 [M+H]⁺.

### Step 8. 5-[1-(Benzenesulfonyl)-2-phenylpyrrolo[2,3-b]pyridin-4-yl]-2-{3-[(tert-butyldimethylsilyl)oxy]propyl}-4-(4-fluorophenyl)pyrazole-3-carbaldehyde

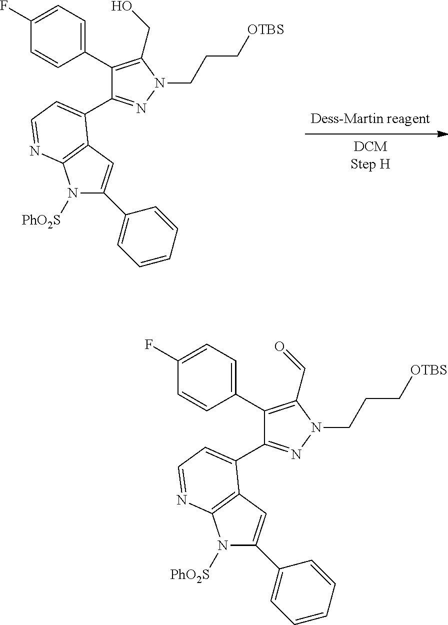

To a stirred mixture of {5-[1-(benzenesulfonyl)-2-phenylpyrrolo[2,3-b]pyridin-4-yl]-2-{3-[(tert-butyldimethylsilyl)oxy]propyl}-4-(4-fluorophenyl)pyrazol-3-yl}methanol (320 mg, 0.46 mmol, 1 equiv) in DCM (12 mL) was added Dess-Martin (390 mg, 0.92 mmol, 2 equiv) in portions at 0° C. under nitrogen atmosphere. The resulting mixture was stirred for 16 h at room temperature under nitrogen atmosphere then concentrated under reduced pressure. The residue was diluted with water (10 mL) and extracted with EtOAc (3×15 mL). The combined organic layers were washed with brine (3×15 mL), dried over anhydrous Na₂SO₄. After filtration, the filtrate was concentrated under reduced pressure. The residue was purified by silica gel column chromatography, eluted with PE/EtOAc (1/1) to afford the title compound (261 mg, 82%) as a white solid. m/z (ESI+ve ion)=695.30 [M+H]⁺.

### Step 9. 5-[1-(Benzenesulfonyl)-2-phenylpyrrolo[2,3-b]pyridin-4-yl]-2-{3-[(tert-butyldimethylsilyl)oxy]propyl}-4-(4-fluorophenyl)pyrazole-3-carbonitrile

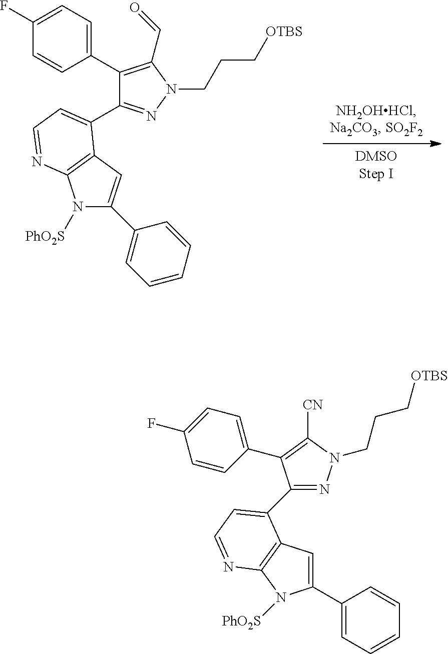

To a stirred mixture of 5-[1-(benzenesulfonyl)-2-phenylpyrrolo[2,3-b]pyridin-4-yl]-2-{3-[(tert-butyldimethylsilyl)oxy]propyl}-4-(4-fluorophenyl)pyrazole-3-carbaldehyde (261 mg, 0.376 mmol, 1 equiv) and Na₂CO₃ (29.9 mg, 0.28 mmol, 0.75 equiv) in DMSO (2 mL) was added NH₂OH·HCl (39.2 mg, 0.56 mmol, 1.5 equiv) at room temperature under nitrogen atmosphere. The resulting mixture was stirred for 1 h at room temperature under nitrogen atmosphere. To the above mixture was added Na₂CO₃ (199 mg, 1.88 mmol, 5 equiv) in portions at room temperature. The resulting mixture was stirred for 16 h at room temperature under SO₂F₂ atmosphere. The resulting mixture was diluted with water (8 mL) and then extracted with EtOAc (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EtOAc (1/1) to afford the title compound (200 mg, 77%) as a white solid. m/z (ESI, +ve ion)=692.3 [M+H]⁺.

### Step 10. 5-[1-(Benzenesulfonyl)-2-phenylpyrrolo[2,3-b]pyridin-4-yl]-4-(4-fluorophenyl)-2-(3-hydroxypropyl)pyrazole-3-carbonitrile

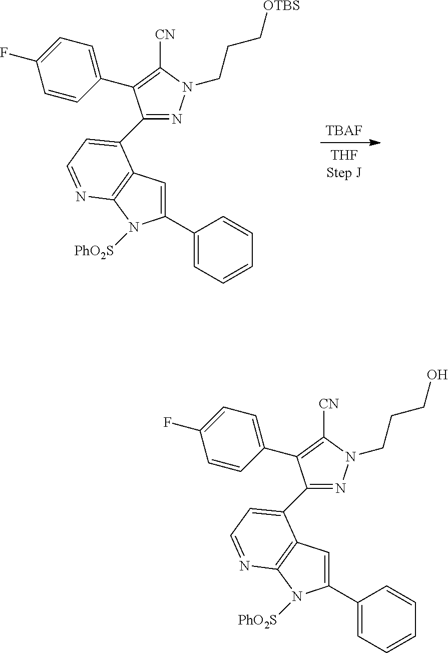

To a stirred mixture of 5-[1-(benzenesulfonyl)-2-phenylpyrrolo[2,3-b]pyridin-4-yl]-2-{3-[(tert-butyldimethylsilyl)oxy]propyl}-4-(4-fluorophenyl)pyrazole-3-carbonitrile (200 mg, 0.289 mmol, 1 equiv) in THF (2 mL) was added TBAF (0.29 mL, 0.289 mmol, 1 equiv, 1 M in THF) dropwise at room temperature under nitrogen atmosphere. The resulting mixture was stirred for 16 h at room temperature under nitrogen atmosphere then diluted with water (5 mL). The resulting mixture was extracted with EtOAc (3×5 mL). The combined organic layers were washed with brine (3×7 mL), dried over anhydrous Na₂SO₄. After filtration, the filtrate was concentrated under reduced pressure. The residue was purified by silica gel column chromatography, eluted with PE/EtOAc (1/1) to afford the title compound (80 mg, 48%) as a white solid. m/z (ESI+ve ion)=578.20 [M+H]⁺.

### Step 11. 4-(4-Fluorophenyl)-1-(3-hydroxypropyl)-3-(2-phenyl-1H-pyrrolo[2,3-b]pyridin-4-yl)-1H-pyrazole-5-carbonitrile

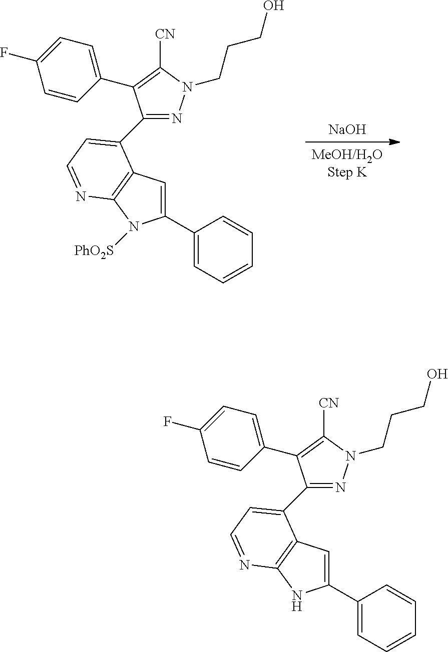

To a stirred mixture of 5-[1-(benzenesulfonyl)-2-phenylpyrrolo[2,3-b]pyridin-4-yl]-4-(4-fluorophenyl)-2-(3-hydroxypropyl)pyrazole-3-carbonitrile (80 mg, 0.14 mmol, 1 equiv) in MeOH (4 mL) was added a solution of NaOH (96 mg, 2.40 mmol, 17.3 equiv) in H₂O (0.4 mL) dropwise at room temperature. The resulting mixture was stirred for 1 h at 50° C. then concentrated under reduced pressure. The resulting mixture was diluted with water (5 mL) and then extracted with EtOAc (3×5 mL). The combined organic layers were washed with brine (3×8 mL), dried over anhydrous Na₂SO₄. After filtration, the filtrate was concentrated under reduced pressure. The crude product (69 mg) was purified by prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 44% B to 54% B in 10 min, 54% B; Wavelength: 254 nm; RT1 (min): 7 to afford Example 16 (43 mg, 71%) as a white solid. m/z (ESI, +ve ion)=438.20 [M+H]⁺. ¹H NMR (400 MHz, DMSO-d₆) δ 12.16 (s, 1H), 8.15 (d, J=5.2 Hz, 1H), 7.87-7.80 (m, 2H), 7.51-7.37 (m, 4H), 7.36 (t, J=7.6 Hz, 1H), 7.35-7.25 (m, 2H), 6.87 (d, J=5.0 Hz, 1H), 6.78 (s, 1H), 4.74 (t, J=5.2 Hz, 1H), 4.56 (t, J=7.2 Hz, 2H), 3.59-3.55 (m, 2H), 2.20-2.14 (m, 2H).

### Example 17. 3-{3-(4-Fluorophenyl)-4-[2-(1,3,4-oxadiazol-2-yl)-1H-pyrrolo[2,3-b]pyridin-4-yl]-1H-pyrazol-1-yl}propan-1-ol

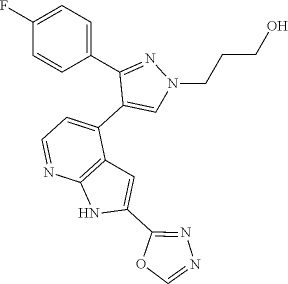 [SMILES: OCCCn1cc(-c2ccnc3[nH]c(-c4nnco4)cc23)c(-c2ccc(F)cc2)n1]

### Step 1. 4-(3-(4-Fluorophenyl)-1-(3-((triisopropylsilyl)oxy)propyl)-1H-pyrazol-4-yl)-1-(phenylsulfonyl)-1H-pyrrolo[2,3-b]pyridine-2-carboxylic acid

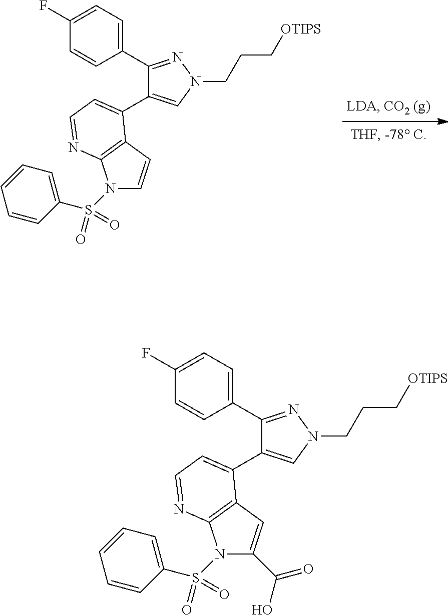

Lithium diisopropylamide (94.8 μL, 190 μmol, 1.2 equiv.) was added to a solution of 4-(3-(4-fluorophenyl)-1-(3-((triisopropylsilyl)oxy)propyl)-1H-pyrazol-4-yl)-1-(phenylsulfonyl)-1H-pyrrolo[2,3-b]pyridine (Intermediate J, 100 mg, 158 μmol) in THF (1.00 mL) at −78° C. The mixture was stirred 1 h at −78° C. then the reaction mixture was degassed with CO₂ gas (from dry ice). The mixture was stirred 10 min at −78° C., warmed up to room temperature then slowly diluted with a saturated solution of NH₄Cl. The aqueous layer was extracted with EtOAc (3×10 mL). The combined organic layers were washed with brine, dried over Na₂SO₄, filtered, and concentrated to provide the title compound (88.0 mg, 82%) as a solid. m/z (ESI, +ve ion)=677.3 [M+H]⁺.

### Step 2. 4-(3-(4-Fluorophenyl)-1-(3-((triisopropylsilyl)oxy)propyl)-1H-pyrazol-4-yl)-1-(phenylsulfonyl)-1H-pyrrolo[2,3-b]pyridine-2-carboxylic acid

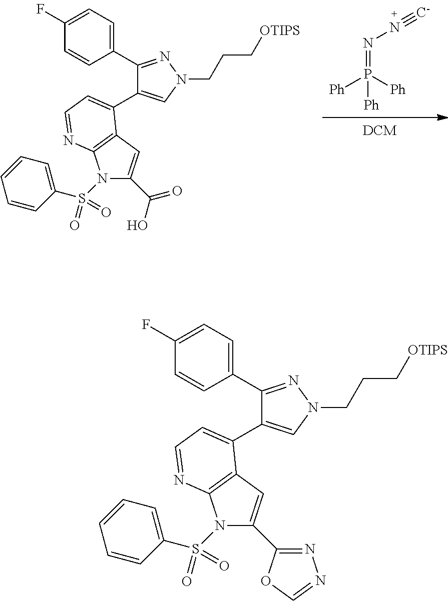

A solution of 4-(3-(4-fluorophenyl)-1-(3-((triisopropylsilyl)oxy)propyl)-1H-pyrazol-4-yl)-1-(phenylsulfonyl)-1H-pyrrolo[2,3-b]pyridine-2-carboxylic acid (170 mg, 211 μmol) in DCM (1.85 mL) was slowly added to a mixture of (isocyanoimino)triphenylphosphorane (65.1 mg, 211 μmol, 1 equiv.) in DCM (1.85 mL) at room temperature. The mixture was stirred for 24 hours at room temperature then an additional 24 hours at 40° C. The mixture was directly loaded on the column and purified by column chromatography (EtOAc in hexanes, 0-50%, a gradient elution) to provide the title compound (47.0 mg, 32%) as a solid. m/z (ESI, +ve ion)=701.5 [M+H]⁺.

### Step 3. 2-(4-(3-(4-Fluorophenyl)-1-(3-((triisopropylsilyl)oxy)propyl)-1H-pyrazol-4-yl)-1-(phenylsulfonyl)-1H-pyrrolo[2,3-b]pyridin-2-yl)-1,3,4-oxadiazole

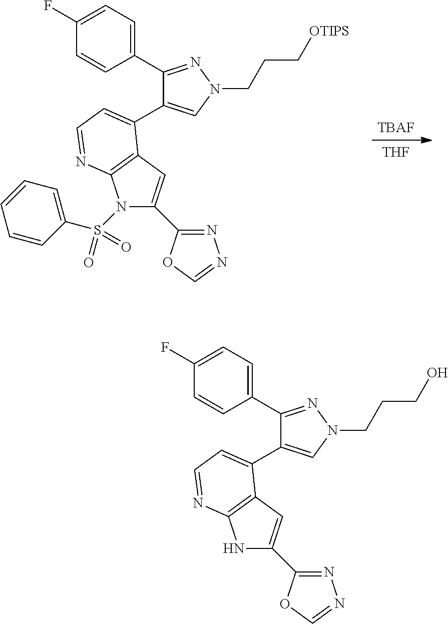

Tetrabutylammonium fluoride (57.1 μL, 57.1 μmol, 1.0 M in THF, 2.0 equiv.) was added to a mixture of 4-(3-(4-fluorophenyl)-1-(3-((triisopropylsilyl)oxy)propyl)-1H-pyrazol-4-yl)-1-(phenylsulfonyl)-1H-pyrrolo[2,3-b]pyridine-2-carboxylic acid (20.0 mg, 28.5 μmol) in THF (384 μL). The mixture was stirred for 15 min at room temperature, then diluted with a saturated solution of NH₄Cl (10 mL) and extracted with EtOAc (3×10 mL). The combined organic layers were washed with brine, dried over Na₂SO₄, filtered, and concentrated under reduced pressure. The residue was purified by reverse phase chromatography (C18 column, MeCN in water with 10 mM ammonium formate, 10-60%, a gradient elution) to provide the titled compound (2.20 mg, 19%) as a solid. ¹H NMR (400 MHz, DMSO-d₆) δ 9.39 (s, 1H), 8.32 (s, 1H), 8.28 (d, J=4.9 Hz, 1H), 7.41 (dd, J=8.6, 5.5 Hz, 2H), 7.17 (d, J=8.9 Hz, 2H), 7.02 (s, 1H), 6.89 (d, J=4.9 Hz, 1H), 4.68 (t, J=5.0 Hz, 1H), 4.31 (t, J=7.0 Hz, 2H), 3.52-3.46 (m, 2H), 2.09-2.00 (m, 2H). m/z (ESI, +ve ion)=405.2 [M+H]⁺.

### Example 18. 3-[4-(4-Fluorophenyl)-5-{2-[4-(4-methylpiperazin-1-yl)phenyl]-1H-pyrrolo[2,3-b]pyridin-4-yl}-2H-1,2,3-triazol-2-yl]propan-1-ol

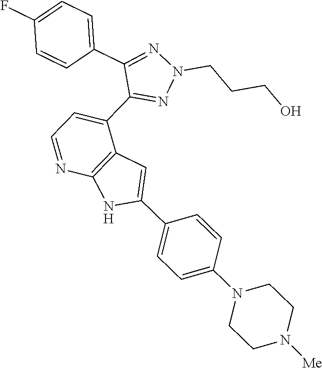 [SMILES: CN1CCN(c2ccc(-c3cc4c(-c5nn(CCCO)nc5-c5ccc(F)cc5)ccnc4[nH]3)cc2)CC1]

### Step 1. 1-(Benzenesulfonyl)-4-[2-(4-fluorophenyl)ethynyl]pyrrolo[2,3-b]pyridine

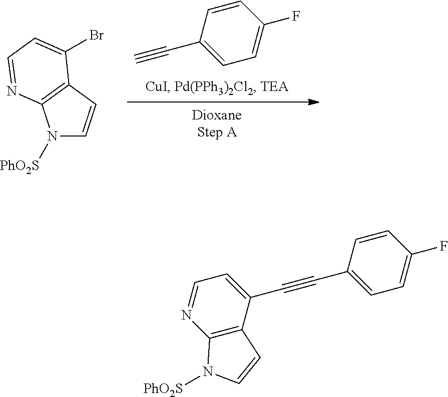

To a stirred mixture of 1-(benzenesulfonyl)-4-bromopyrrolo[2,3-b]pyridine (Intermediate C, 3.3 g, 9.79 mmol, 1.00 equiv) and 1-ethynyl-4-fluorobenzene (2.35 g, 19.6 mmol, 2 equiv) in dioxane (50 mL) were added CuI (75 mg, 0.39 mmol, 0.04 equiv), TEA (2.48 g, 24.468 mmol, 2.5 equiv) and Pd(PPh₃)₂Cl₂ (137 mg, 0.20 mmol, 0.02 equiv) at room temperature under nitrogen atmosphere. The resulting mixture was stirred for 16 h at 90° C. under nitrogen atmosphere then concentrated under reduced pressure. The residue was purified by silica gel column chromatography, eluted with PE/EtOAc (3/1) to afford the title compound (2 g, 54%) as a yellow solid. m/z (ESI, +ve ion)=377.10 [M+H]⁺.

### Step 2. 4-[1-(Benzenesulfonyl)pyrrolo[2,3-b]pyridin-4-yl]-5-(4-fluorophenyl)-2H-1,2,3-triazole

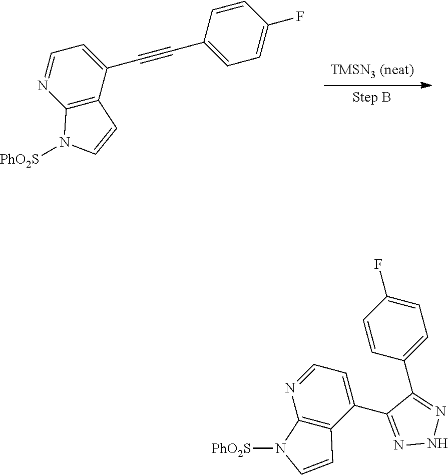

A mixture of 1-(benzenesulfonyl)-4-[2-(4-fluorophenyl)ethynyl]pyrrolo[2,3-b]pyridine (750 mg, 1.99 mmol, 1.00 equiv) in TMSN₃ (2.00 mL, 15.2 mmol, 7.63 equiv) was stirred for 2 days at 140° C. under nitrogen atmosphere. The resulting mixture was diluted with CHCl₃/EtOAc (1/1, 10 mL). The precipitated solids were collected by filtration to afford the title compound (400 mg, 48%) as an off-white solid. m/z (ESI, +ve ion)=420.10 [M+H]⁺.

### Step 3. 4-[1-(Benzenesulfonyl)pyrrolo[2,3-b]pyridin-4-yl]-2-{4-[(tert-butyldimethylsilyl)oxy]butyl}-5-(4-fluorophenyl)-1,2,3-triazole

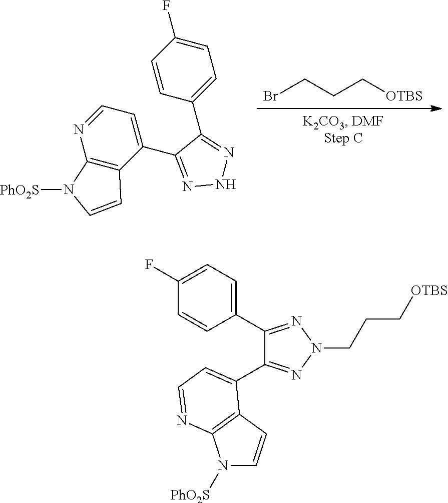

To a stirred mixture of 4-[1-(benzenesulfonyl)pyrrolo[2,3-b]pyridin-4-yl]-5-(4-fluorophenyl)-2H-1,2,3-triazole (222 mg, 0.53 mmol, 1.00 equiv) and (3-bromopropoxy)(tert-butyl)dimethylsilane (201 mg, 0.79 mmol, 1.5 equiv) in DMF (5 mL) was added K₂CO₃ (146.30 mg, 1.058 mmol, 2 equiv) at 0° C. under nitrogen atmosphere. The resulting mixture was stirred for 6 h at 0° C. under nitrogen atmosphere then diluted with ethyl acetate (30 mL), washed with brine (5×5 mL), dried over anhydrous Na₂SO₄. After filtration, the filtrate was concentrated under reduced pressure. The residue was purified by silica gel column chromatography, eluted with PE/EtOAc (5/1) to afford the title compound (240 mg, 75%) as a yellow solid. m/z (ESI, +ve ion)=592.21 [M+H]⁺.

### Step 4. 4-[1-(Benzenesulfonyl)-2-bromopyrrolo[2,3-b]pyridin-4-yl]-2-{3-[(tert-butyldimethylsilyl)oxy]propyl}-5-(4-fluorophenyl)-1,2,3-triazole

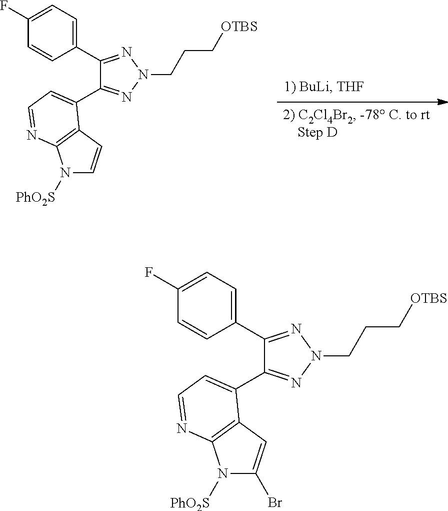

To a mixture of 4-[1-(benzenesulfonyl)pyrrolo[2,3-b]pyridin-4-yl]-2-{3-[(tert-butyldimethylsilyl)oxy]propyl}-5-(4-fluorophenyl)-1,2,3-triazole (74 mg, 0.13 mmol, 1 equiv) in THF (1 mL) was added n-BuLi (2.5 M in hexane, 0.055 mL, 0.13 mmol, 1.2 equiv) at −78° C. After stirred for 1 h at −78° C., a solution of 1,2-dibromo-1,1,2,2-tetrachloroethane (49 mg, 0.15 mmol, 1.2 equiv) in THF (0.5 mL) was added into the above mixture at −78° C. The resulting mixture was stirred for 1 h at −78° C. and then warm up to room temperature. The reaction was quenched with sat. aqueous of NH₄Cl (5 mL). The mixture was extracted with EtOAc (3×10 mL), the combined organic layer was washed with brine (2×5 mL), dried over anhydrous Na₂SO₄ and then concentrated under reduced pressure to afford the title compound (80 mg, 76%, 80% purity) as a yellow oil. m/z (ESI, +ve ion)=670.15, 672.15 [M+H]⁺.

### Step 5. 1-{4-[1-(Benzenesulfonyl)-4-(2-{3-[(tert-butyldimethylsilyl)oxy]propyl}-5-(4-fluorophenyl)-1,2,3-triazol-4-yl)pyrrolo[2,3-b]pyridin-2-yl]phenyl}-4-methylpiperazine

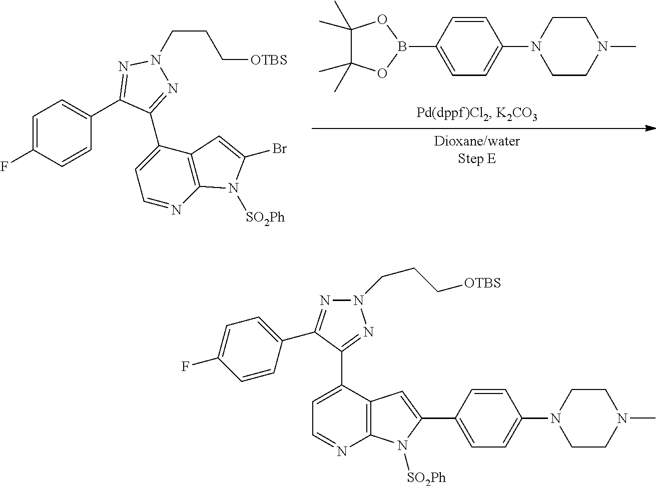

To a stirred mixture of 4-[1-(benzenesulfonyl)-2-bromopyrrolo[2,3-b]pyridin-4-yl]-2-{3-[(tert-butyldimethylsilyl)oxy]propyl}-5-(4-fluorophenyl)-1,2,3-triazole (104 mg, 0.155 mmol, 1 equiv) and 1-methyl-4-[4-(4,4,5,5-tetramethyl-1,3,2-dioxaborolan-2-yl)phenyl]piperazine (70.3 mg, 0.23 mmol, 1.5 equiv) in dioxane (4 mL) and water (1 mL) were added Pd(dppf)Cl₂·CH₂Cl₂ (12.63 mg, 0.015 mmol, 0.1 equiv) and Na₂CO₃ (49.3 mg, 0.47 mmol, 3.00 equiv) at room temperature under nitrogen atmosphere. The resulting mixture was stirred for 3 h at 80° C. under nitrogen atmosphere then filtered and the filter cake was washed with EtOAc (3×5 mL). The filtrate was concentrated under reduced pressure. The residue was purified by prep-TLC (DCM/MeOH=10/1) to afford the title compound (70 mg, 59%) as a yellow solid. m/z (ESI, +ve ion)=766.40 [M+H]⁺.

### Step 6. 3-[4-(4-Fluorophenyl)-5-{2-[4-(4-methylpiperazin-1-yl)phenyl]-1H-pyrrolo[2,3-b]pyridin-4-yl}-2H-1,2,3-triazol-2-yl]propan-1-ol

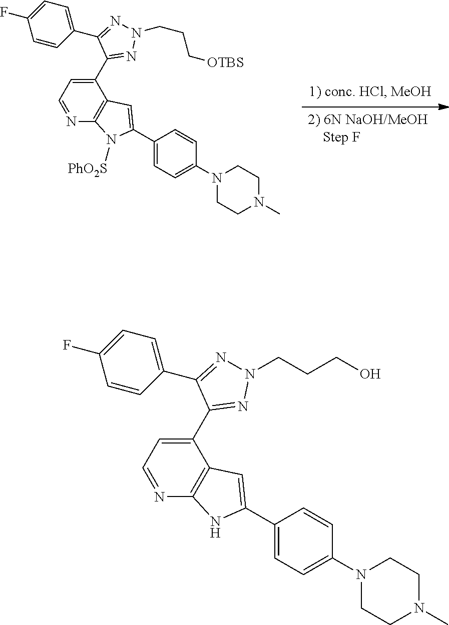

To a stirred mixture of 1-{4-[1-(benzenesulfonyl)-4-(2-{3-[(tert-butyldimethylsilyl)oxy]propyl}-5-(4-fluorophenyl)-1,2,3-triazol-4-yl)pyrrolo[2,3-b]pyridin-2-yl]phenyl}-4-methylpiperazine (80 mg, 0.104 mmol, 1 equiv) in methanol (2 mL) was added conc. HCl (0.21 mL, 2.080 mmol, 20.00 equiv, 37%) at room temperature. The resulting mixture was stirred for 2 h at room temperature then concentrated under vacuum. The residue was dissolved in methanol (2 mL), to the above mixture was added 6 N NaOH (0.17 mL, 1.040 mmol, 10 equiv) at room temperature. The resulting mixture was stirred for additional 30 min at 50° C. The crude product was purified by prep-HPLC with the following conditions: Column: YMC-Actus Triart C18 ExRS, 30×150 mm, 5 μm; Mobile Phase A: water (10 mmol/L NH₄HCO₃), Mobile Phase B: ACN; Flow rate: 60 mL/min; Gradient: 35% B to 45% B in 8 min, 45% B; Wavelength: 254 nm; RT1 (min): 7 to afford Example 18 (12 mg, 22%) as a yellow solid. m/z (ESI, +ve ion)=512.25 [M+H]⁺. ¹H-NMR (400 MHz, DMSO-d₆) δ 12.10 (s, 1H), 8.14 (d, J=5.2 Hz, 1H), 7.69 (d, J=8.8 Hz, 2H), 7.26-7.21 (m, 2H), 7.53-7.49 (m, 2H), 7.00-6.97 (m, 3H), 6.53 (s, 1H), 4.71 (t, J=4.8 Hz, 1H), 4.64 (t, J=7.2 Hz, 2H), 3.58-3.54 (m, 2H), 3.22 (t, J=5.2 Hz, 4H), 2.45 (s, 4H), 2.23 (s, 3H), 2.20-2.14 (m, 2H).

### Example 19. 3-[3-(4-Fluorophenyl)-4-{6-[4-(4-methylpiperazin-1-yl)phenyl]-7H-pyrrolo[2,3-d]pyrimidin-4-yl}-1H-pyrazol-1-yl]propan-1-ol

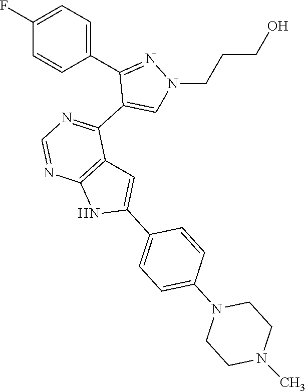 [SMILES: CN1CCN(c2ccc(-c3cc4c(-c5cn(CCCO)nc5-c5ccc(F)cc5)ncnc4[nH]3)cc2)CC1]

### Step 1. 1-Methyl-4-(4-(4,4,5,5-tetramethyl-1,3,2-dioxaborolan-2-yl)phenyl)piperazine

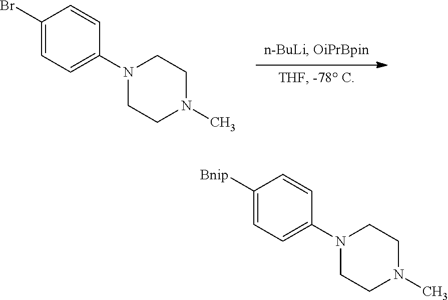

n-Butyllithium (346 μL, 864 μmol, 1.5 equiv., 2.5 M in hexane) was added to mixture of 1-(4-bromophenyl)-4-methylpiperazine (150 mg, 576 μmol) in THF (9.32 mL) at −78° C. The mixture was stirred for 30 min at −78° C. 2-Isopropoxy-4,4,5,5-tetramethyl-1,3,2-dioxaborolane (124 μL, 593 μmol, 1.03 equiv.) was added and the mixture was stirred for 2 h at −78° C. The mixture was diluted with water (30 mL) and extracted with EtOAc (3×20 mL). The combined organic layers were washed with brine, then dried over Na₂SO₄, filtered, and concentrated to provide the title compound (220 mg, quant.) as a solid. The material was used in the next steps without further purification. m/z (ESI, +ve ion)=303.5 [M+H]⁺.

### Step 2. 4-Chloro-6-(4-(4-methylpiperazin-1-yl)phenyl)-7H-pyrrolo[2,3-d]pyrimidine

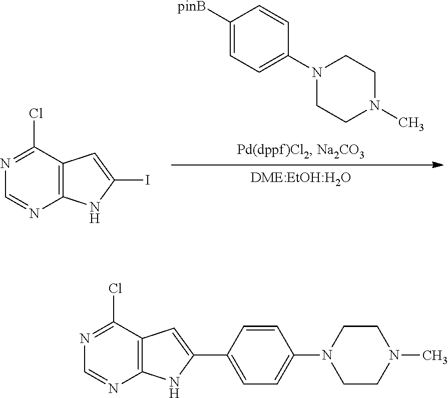

1,1′-Bis(diphenylphosphino)ferrocene dichloropalladium (II) (31.8 mg, 43.4 μmol, 0.1 equiv.) was added to a degassed mixture of 4-chloro-6-iodo-7H-pyrrolo[2,3-d]pyrimidine (124 mg, 434 μmol), 1-methyl-4-(4-(4,4,5,5-tetramethyl-1,3,2-dioxaborolan-2-yl)phenyl)piperazine (210 mg, 694 μmol, 1.6 equiv.) and sodium carbonate (46.5 mg, 434 μmol, 1.0 equiv.) in a mixture of DME (1.58 mL), EtOH (676 μL) and H₂O (225 μL). The mixture was stirred for 1 hour at 110° C. in a microwave reactor, then diluted with EtOAc (20 mL). The mixture was filtered and the filtrate was concentrated under reduced pressure. The residue was purified by column chromatography (MeOH in DCM, 0-20%, a gradient elution), to provide the title compound (87.0 mg, 61%) as a solid. ¹H NMR (400 MHz, DMSO-d₆) δ 12.91-12.78 (br s, 1H), 8.54 (s, 1H), 7.92 (d, J=8.8 Hz, 2H), 7.12 (d, J=8.9 Hz, 2H), 6.96 (d, J=2.0 Hz, 1H), 3.18-3.00 (m, 8H), 2.80 (br s, 3H). m/z (ESI, +ve ion)=328.1 [M+H]⁺.

### Step 3. 4-Chloro-6-(4-(4-methylpiperazin-1-yl)phenyl)-7H-pyrrolo[2,3-d]pyrimidine

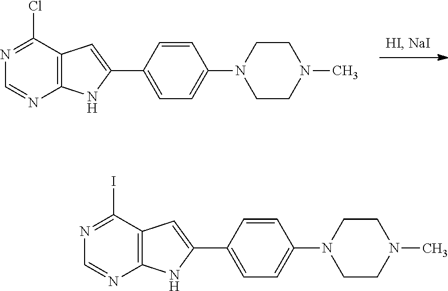

4-Chloro-6-(4-(4-methylpiperazin-1-yl)phenyl)-7H-pyrrolo[2,3-d]pyrimidine (200 mg, 871 μmol) was suspended in hydriodic acid (2.97 mL, 13.1 mmol, 15 equiv.) at 0° C. and the mixture was stirred at 0° C. for 3 h. Sodium iodide (121 mg, 796 μmol, 0.91 equiv.) was added and the mixture was heated to 60° C. for 4 h. The reaction mixture was diluted with ice/water (5 mL). The mixture was stirred vigorously until all of the ice had melted. 15% w/w NaOH solution in water was added until a pH of 6-7 was obtained. EtOAc (20 mL) and water (10 mL) were added and the layers were separated. The aqueous layer was extracted with additional EtOAc (3×20 mL) and the combined organic layers were washed with water (10 mL), brine (10 mL), dried over Na₂SO₄, filtered, and concentrated under reduced pressure to provide the title compound (193 mg, 69%) as a solid. m/z (ESI, +ve ion)=420.1 [M+H]⁺.

### Step 4. 4-Iodo-6-(4-(4-methylpiperazin-1-yl)phenyl)-7H-pyrrolo[2,3-d]pyrimidine

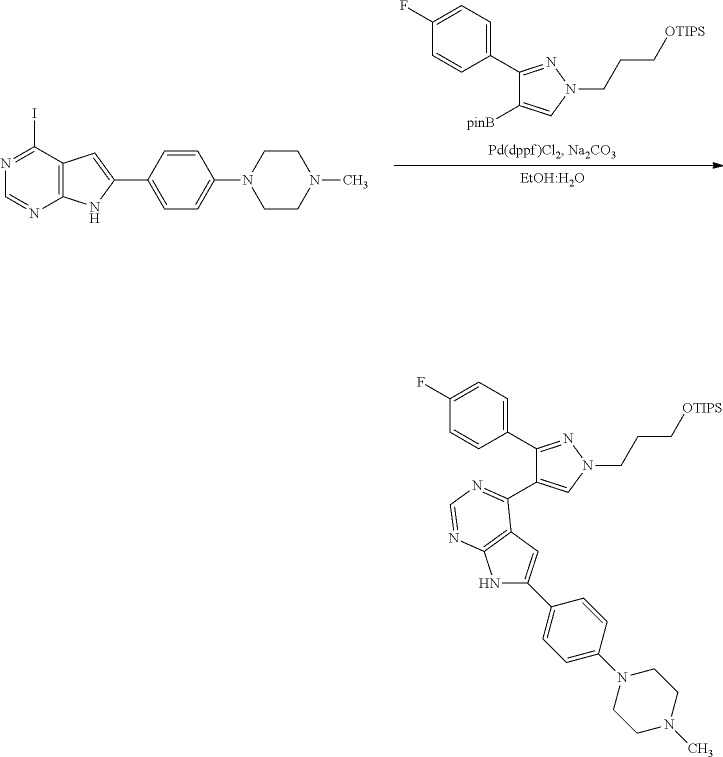

1,1′-Bis(diphenylphosphino)ferrocene dichloropalladium (II) (9.79 mg, 12.7 μmol, 0.1 equiv.) was added to a degassed mixture of 4-chloro-6-(4-(4-methylpiperazin-1-yl)phenyl)-7H-pyrrolo[2,3-d]pyrimidine (55.0 mg, 131 μmol), 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 98.9 mg, 197 μmol, 1.5 equiv.) and sodium carbonate (42.1 mg, 394 μmol, 3.0 equiv.) in a mixture of EtOH (990 μL) and H₂O (240 μL). The mixture was stirred for 10 minutes at 140° C. in a microwave reactor, then diluted with EtOAc. The mixture was filtered and the filtrate was concentrated under reduced pressure to provide the title compound (87.5 mg, quant.) as a solid, which was used in the next steps without further purification. m/z (ESI, +ve ion)=668.5 [M+H]⁺.

### Step 5. 3-[3-(4-Fluorophenyl)-4-{6-[4-(4-methylpiperazin-1-yl)phenyl]-7H-pyrrolo[2,3-d]pyrimidin-4-yl}-1H-pyrazol-1-yl]propan-1-ol

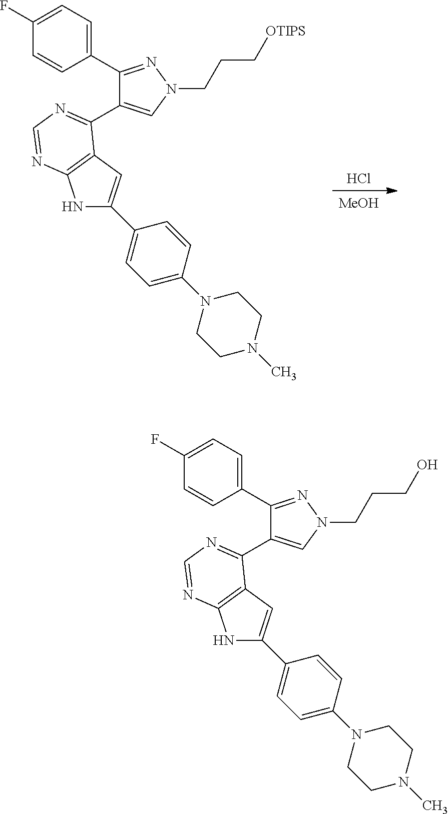

Hydrochloric acid (656 μL, 656 μmol, 1 M in water, 5 equiv.) was added to a mixture of 4-iodo-6-(4-(4-methylpiperazin-1-yl)phenyl)-7H-pyrrolo[2,3-d]pyrimidine (87.5 mg, 131 μmol) in THF (500 μL) and MeOH (500 μL). The resulting mixture was stirred at room temperature for 16 h. The pH of the reaction mixture was adjusted to 7-8 with 15% w/w NaOH, then EtOAc (10 mL) was added. The aqueous layer was extracted with EtOAc (3×10 mL) and the combined organic layers were washed with H₂O (10 mL), brine (10 mL), dried over Na₂SO₄, filtered and concentrated. The residue was purified by reverse phase chromatography (C18 column, MeCN in water with 10 mM ammonium formate, 5-60%, a gradient elution) to provide the title compound (5.10 mg, 8%) as a solid. ¹H NMR (400 MHz, DMSO-d₆) δ 12.38 (br s, 1H), 8.54 (s, 1H), 8.47 (s, 1H), 7.74 (d, J=8.6 Hz, 2H), 7.66-7.51 (m, 2H), 7.15 (app t, J=8.5 Hz, 2H), 7.01 (d, J=8.2 Hz, 2H), 6.64 (s, 1H), 4.69 (br s, 1H), 4.32 (t, J=6.4 Hz, 2H), 3.50 (m, 2H), 3.23 (s, 4H), 2.45 (s, 4H), 2.22 (s, 3H), 2.10-2.02 (m, 2H). m/z (ESI, +ve ion)=512.3 [M+H]⁺.

### Example 21. 4-[3-(4-Fluorophenyl)-4-(2-phenyl[1,3]oxazolo[5,4-d]pyrimidin-7-yl)-1H-pyrazol-1-yl]-2-methylbutan-2-ol

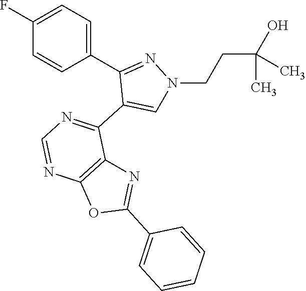 [SMILES: CC(C)(O)CCn1cc(-c2ncnc3oc(-c4ccccc4)nc23)c(-c2ccc(F)cc2)n1]

### Step 1. 4-Bromo-2-methylbutan-2-ol

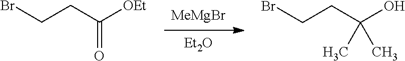

A solution of methylmagnesium bromide (9.02 mL, 27.1 mmol) was added dropwise to a solution of ethyl 3-bromopropionate (1.42 mL, 10.8 mmol, 2.5 equiv.) in anhydrous Et₂O (86.0 mL) at 0° C. The reaction mixture was stirred at 0° C. for 2 hours then warmed to room temperature over 16 hours. The reaction mixture was cooled to 0° C., and water (25 mL) was slowly added. After stirring for 15 minutes, the layers were separated, and the organic layer was washed with water (30 mL). The organic layer was dried over MgSO₄, filtered, and concentrated under reduced pressure to provide the title compound (1.75 g, 97%) as an oil. The material was used in the next steps without further purification. ¹H NMR (400 MHz, CDCl₃) δ 3.54-3.45 (m, 2H), 2.16-2.04 (m, 2H), 1.62 (br s, 1H), 1.25 (s, 6H).

### Step 2. 4-(3-(4-Fluorophenyl)-4-(2-phenyloxazolo[5,4-d]pyrimidin-7-yl)-1H-pyrazol-1-yl)-2-methylbutan-2-ol

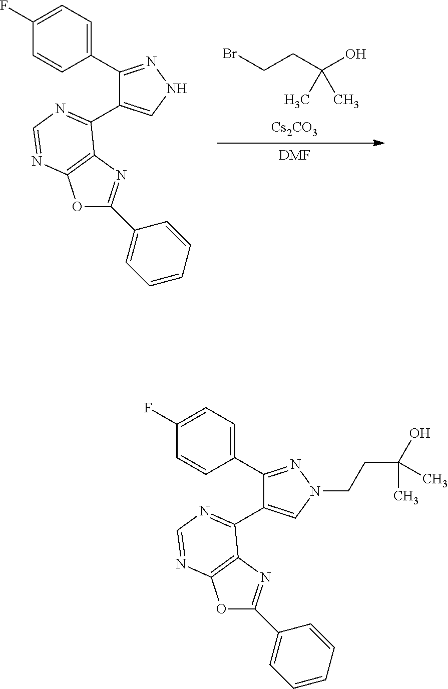

Cesium carbonate (92.1 mg, 280 μmol, 2 equiv.) was added to a mixture of 7-(3-(4-fluorophenyl)-1H-pyrazol-4-yl)-2-phenyloxazolo[5,4-d]pyrimidine (Intermediate L, 50.0 mg, 140 μmol) and 4-bromo-2-methylbutan-2-ol (35.8 mg, 214 μmol, 1.5 equiv.) in DMF (700 μL). The mixture was stirred for 15 h at 60° C. then cooled to room temperature and diluted with water (10 mL). The mixture was extracted with EtOAc (3×10 mL). The combined organic layers were washed with brine, then dried (Na₂SO₄), filtered and concentrated. The residue was purified by column chromatography (MeOH in DCM, 0-5%, a gradient elution). The impure fractions were combined and purified by preparative TLC (2% MeOH in DCM) and further purified by reverse phase chromatography (C18 column, 30-100% MeCN in water with 10 mM ammonium formate, a gradient elution) to provide the title compound (11.0 mg, 16%) as a solid. ¹H NMR (400 MHz, DMSO-d₆) δ 8.95 (s, 1H), 8.78 (s, 1H), 8.30-8.24 (m, 2H), 7.76-7.63 (m, 5H), 7.20 (t, J=8.9 Hz, 2H), 4.54 (s, 1H), 4.45-4.36 (m, 2H), 2.10-2.00 (m, 2H), 1.19 (s, 6H). m/z (ESI, +ve ion)=444.2 [M+H]⁺.

### Example 23. 4-[4-(4-Fluorophenyl)-5-(2-phenyl-1H-pyrrolo[2,3-b]pyridin-4-yl)-2H-1,2,3-triazol-2-yl]butan-1-ol

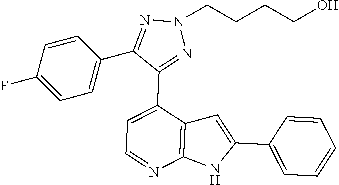 [SMILES: OCCCCn1nc(-c2ccc(F)cc2)c(-c2ccnc3[nH]c(-c4ccccc4)cc23)n1]

### Step 1. 1-(Benzenesulfonyl)-4-[2-(4-fluorophenyl)ethynyl]-2-phenylpyrrolo[2,3-b]pyridine

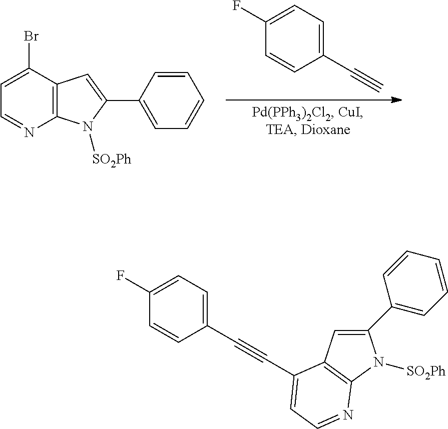

To a stirred mixture of 1-(benzenesulfonyl)-4-bromo-2-phenylpyrrolo[2,3-b]pyridine (Intermediate E, 412 mg, 0.997 mmol, 1 equiv) and 1-ethynyl-4-fluorobenzene (239.50 mg, 1.994 mmol, 2 equiv) in 1,4-dioxane (4 mL) were added Pd(PPh₃)₂Cl₂ (70 mg, 0.10 mmol, 0.1 equiv) and CuI (38 mg, 0.20 mmol, 0.2 equiv) at room temperature under nitrogen atmosphere. To the above mixture was added TEA (252 mg, 2.49 mmol, 2.5 equiv) dropwise over 1 min at room temperature under nitrogen atmosphere. The resulting mixture was stirred for overnight at 90° C. under nitrogen atmosphere. The mixture was cooled down to room temperature. The reaction was quenched by the addition of water (40 mL) at room temperature. The resulting mixture was extracted with EtOAc (3×30 mL). The combined organic layers were washed with brine (2×30 mL), dried over anhydrous Na₂SO₄. After filtration, the filtrate was concentrated under reduced pressure. The residue was purified by silica gel column chromatography, eluted with PE/EtOAc (4/1) to afford the title compound (400 mg, 89%) as a yellow solid. m/z (ESI, +ve ion)=453.10 [M+H]⁺.

### Step 2. 4-[1-(Benzenesulfonyl)-2-phenylpyrrolo[2,3-b]pyridin-4-yl]-5-(4-fluorophenyl)-2H-1,2,3-triazole

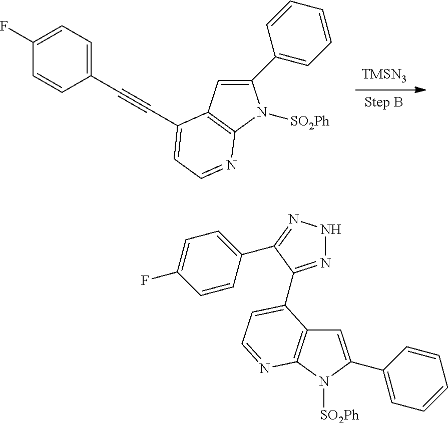

A mixture of 1-(benzenesulfonyl)-4-[2-(4-fluorophenyl)ethynyl]-2-phenylpyrrolo[2,3-b]pyridine (349 mg, 0.77 mmol, 1 equiv) in TMSN₃ (0.7 mL, 6.13 mmol) was stirred for 16 h at 140° C. under nitrogen atmosphere. The reaction was quenched by the addition of water (5 mL) at room temperature. The resulting mixture was extracted with EtOAc (3×10 mL). The combined organic layers were washed with brine (20 mL), dried over anhydrous Na₂SO₄. After filtration, the filtrate was concentrated under reduced pressure. The residue was purified by silica gel column chromatography, eluted with DCM/EtOAc (20/1) to afford the title compound (300 mg, 79%) as an orange solid. m/z (ESI, +ve ion)=496.05 [M+H]⁺.

### Step 3. 4-{4-[1-(Benzenesulfonyl)-2-phenylpyrrolo[2,3-b]pyridin-4-yl]-5-(4-fluorophenyl)-1,2,3-triazol-2-yl}butan-1-ol

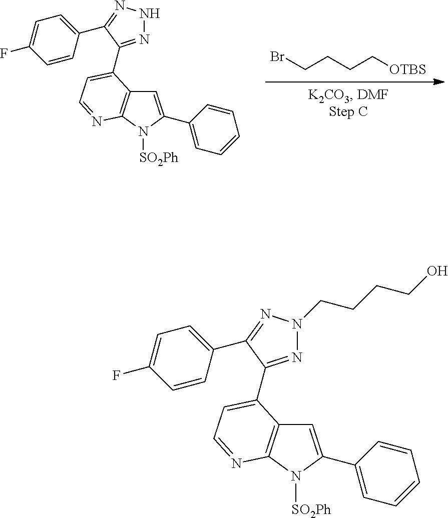

To a stirred mixture of 4-[1-(benzenesulfonyl)-2-phenylpyrrolo[2,3-b]pyridin-4-yl]-5-(4-fluorophenyl)-2H-1,2,3-triazole (100 mg, 0.20 mmol, 1 equiv) and K₂CO₃ (56 mg, 0.40 mmol, 2 equiv) in DMF (1 mL) was added (4-bromobutoxy)(tert-butyl)dimethylsilane (107.88 mg, 0.404 mmol, 2 equiv) dropwise at 0° C. under nitrogen atmosphere. The resulting mixture was stirred overnight at room temperature under nitrogen atmosphere. The reaction was quenched by the addition of water (6 mL) at room temperature and extracted with EtOAc (3×10 mL). The combined organic layers were washed with brine (2×25 mL), dried over anhydrous Na₂SO₄. After filtration, the filtrate was concentrated under reduced pressure. The residue was purified by reverse flash chromatography with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 45% B to 55% B in 8 min, 55% B to 62% B in 10 min, 62% B; Wavelength: 254 nm; RT1 (min): 7.28 to afford the title compound (80 mg, 70%) as a yellow solid. m/z (ESI, +ve ion)=568.30 [M+H]⁺.

### Step 4. 4-[4-(4-Fluorophenyl)-5-(2-phenyl-1H-pyrrolo[2,3-b]pyridin-4-yl)-2H-1,2,3-triazol-2-yl]butan-1-ol

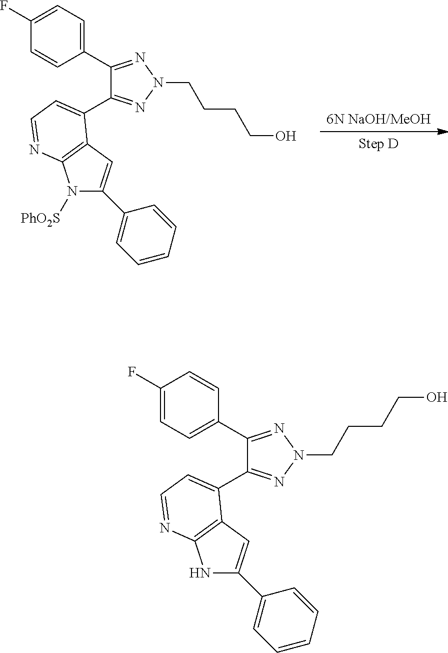

To a stirred mixture of 4-{4-[1-(benzenesulfonyl)-2-phenylpyrrolo[2,3-b]pyridin-4-yl]-5-(4-fluorophenyl)-1,2,3-triazol-2-yl}butan-1-ol (50 mg, 0.088 mmol, 1 equiv) in MeOH (3 mL) and H₂O (0.3 mL) was added NaOH (68.70 mg, 1.716 mmol, 19.5 equiv) at room temperature under nitrogen atmosphere. The resulting mixture was stirred for 30 min at 50° C. under nitrogen atmosphere. The resulting mixture was concentrated under reduced pressure. The residue was purified by reverse flash chromatography with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 40% B to 50% B in 8 min, 50% B; Wavelength: 254 nm; RT1 (min): 8 to afford Example 23 (31 mg, 82%) as a white solid. m/z (ESI, +ve ion)=428.20 [M+H]⁺. ¹H-NMR (400 MHz, DMSO-d₆) δ 12.31 (s, 1H), 8.22 (d, J=4.8 Hz, 1H), 7.85 (d, J=7.6 Hz, 2H), 7.53-7.50 (m, 2H), 7.47-7.43 (m, 2H), 7.37-7.33 (m, 1H), 7.26-7.22 (m, 2H), 7.03 (d, J=5.2 Hz, 1H), 6.75 (d, J=1.6 Hz, 1H), 4.62-4.58 (m, 2H), 4.54-4.51 (m, 1H), 3.49-3.48 (m, 2H), 2.08-2.04 (m, 2H), 1.58-1.51 (m, 2H).

### Example 27. 2-[4-(4-Fluorophenyl)-5-(8-phenyl-9H-purin-6-yl)-2H-1,2,3-triazol-2-yl]ethan-1-ol

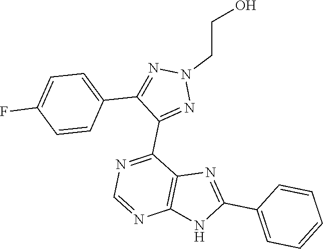 [SMILES: OCCn1nc(-c2ccc(F)cc2)c(-c2ncnc3[nH]c(-c4ccccc4)nc23)n1]

### Step 1. 4-Bromo-2-{2-[(tert-butyldimethylsilyl)oxy]ethyl}-5-(4-fluorophenyl)-1,2,3-triazole

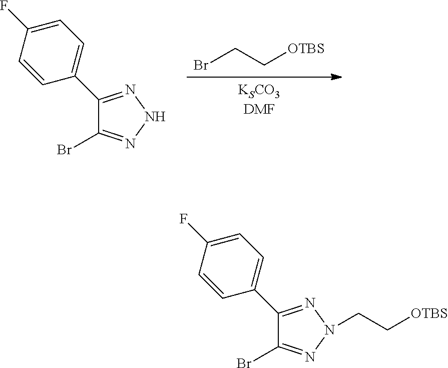

To a stirred solution of 4-bromo-5-(4-fluorophenyl)-2H-1,2,3-triazole (Intermediate M, 500 mg, 2.066 mmol, 1 equiv) and (2-bromoethoxy) (tert-butyl)dimethylsilane (741.25 mg, 3.099 mmol, 1.5 equiv) in DMF (20 mL) was added K₂CO₃ (570.98 mg, 4.132 mmol, 2 equiv) at room temperature under nitrogen atmosphere. The resulting mixture was stirred for 4 h at room temperature under nitrogen atmosphere. The reaction was quenched by the addition of water (50 mL) at room temperature then extracted with EtOAc (3×100 mL). The combined organic layers were washed with brine (5×50 mL), dried over anhydrous Na₂SO₄. After filtration, the filtrate was concentrated under reduced pressure. The residue was purified by silica gel column chromatography, eluted with PE:EtOAc (4:1) to afford the title compound (800 mg, 87%) as a white oil. m/z (ESI, +ve ion)=400.10, 402.10 [M+H]⁺.

### Step 2. 2-{2-[(tert-Butyldimethylsilyl)oxy]ethyl}-4-(4-fluorophenyl)-5-(4,4,5,5-tetramethyl-1,3,2-dioxaborolan-2-yl)-1,2,3-triazole

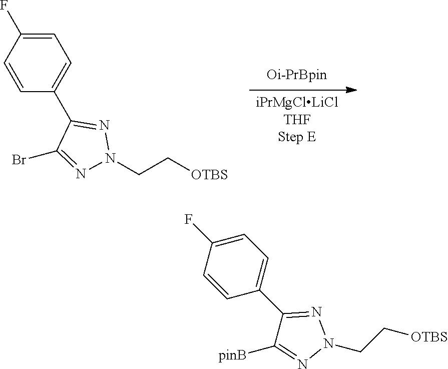

To a stirred solution of 4-bromo-2-{2-[(tert-butyldimethylsilyl)oxy]ethyl}-5-(4-fluorophenyl)-1,2,3-triazole (300 mg, 0.75 mmol, 1 equiv) in THF (1 mL) was added 1 M iso-PrMgCl—LiCl (1.12 mL, 1.12 mmol, 1.5 equiv) dropwise at −78° C. under nitrogen atmosphere. The resulting mixture was stirred for 1 h at −78° C. under nitrogen atmosphere. To the above mixture was added 2-isopropoxy-4,4,5,5-tetramethyl-1,3,2-dioxaborolane (209 mg, 1.12 mmol, 1.5 equiv) dropwise over 2 min at −78° C. The mixture was warmed up to room temperature and stirred for additional 2 h at room temperature. The crude title compound (450 mg, 67%) was used in the next step directly without further purification. m/z (ESI+ve ion)=448.25 [M+H]⁺.

### Step 3. 2-{2-[(tert-Butyldimethylsilyl)oxy]ethyl}-4-(4-fluorophenyl)-5-(4,4,5,5-tetramethyl-1,3,2-dioxaborolan-2-yl)-1,2,3-triazole

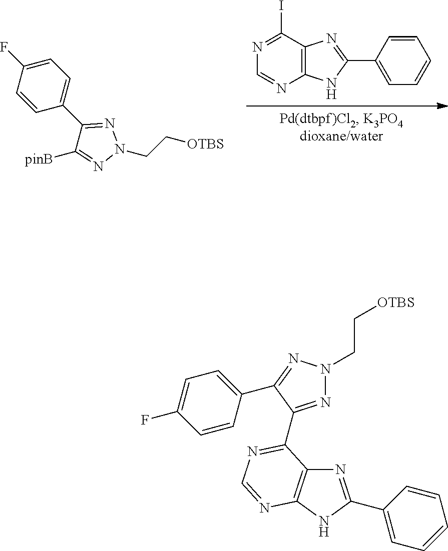

To a stirred solution of 2-{2-[(tert-butyldimethylsilyl)oxy]ethyl}-4-(4-fluorophenyl)-5-(4,4,5,5-tetramethyl-1,3,2-dioxaborolan-2-yl)-1,2,3-triazole (133.35 mg, 0.30 mmol, 1.5 equiv) and 6-iodo-8-phenyl-9H-purine (Intermediate G, 64 mg, 0.20 mmol, 1.0 equiv) in 1,4-dioxane (3 mL) and H₂O (0.6 mL) were added Pd(1,1-bis(di-tert-butylphosphino)ferrocene)Cl₂ (12.95 mg, 0.020 mmol, 0.1 equiv) and K₃PO₄ (84 mg, 0.40 mmol, 2.0 equiv) at room temperature under nitrogen atmosphere. The resulting mixture was stirred for additional 2 h at 90° C. then cooled down to room temperature. The reaction was quenched by the addition of water (10 mL) at room temperature. The resulting mixture was extracted with EtOAc (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE:EtOAc (1:2) to afford the title compound (62 mg, 61%) as a brown oil. m/z (ESI+ve ion)=516.25 [M+H]⁺.

### Step 4. 2-[4-(4-Fluorophenyl)-5-(8-phenyl-9H-purin-6-yl)-1,2,3-triazol-2-yl]ethanol

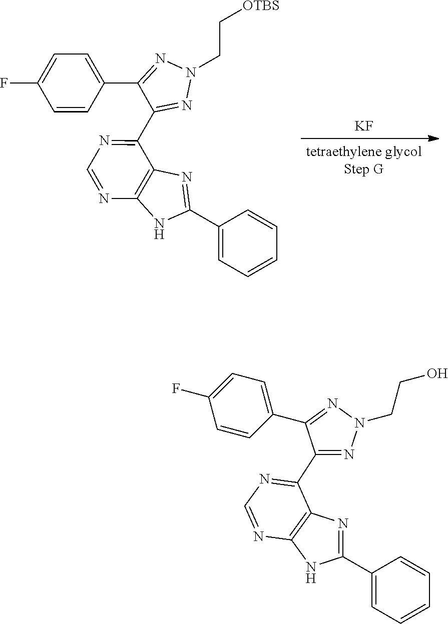 [SMILES: CC(C)(C)[Si](C)(C)OCCn1nc(-c2ccc(F)cc2)c(-c2ncnc3[nH]c(-c4ccccc4)nc23)n1 ; "KF" ; "tetraethylene glycolStep G" ; OCCn1nc(-c2ccc(F)cc2)c(-c2ncnc3[nH]c(-c4ccccc4)nc23)n1]

A solution of 2-{2-[(tert-butyldimethylsilyl)oxy]ethyl}-4-(4-fluorophenyl)-5-(4,4,5,5-tetramethyl-1,3,2-dioxaborolan-2-yl)-1,2,3-triazole (62 mg, 0.12 mmol, 1 equiv) and KF (7.0 mg, 0.12 mmol, 1 equiv) in tetraethylene glycol (3 mL) was stirred for 3 h at 60° C. under nitrogen atmosphere. The reaction was quenched by the addition of water (6 mL) at room temperature. The resulting mixture was extracted with EtOAc (3×10 mL). The combined organic layers were washed with brine (10 mL), dried over anhydrous Na₂SO₄. After filtration, the filtrate was concentrated under reduced pressure. The crude product (70 mg) was purified by prep-HPLC with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 25% B to 45% B in 8 min, 45% B; Wavelength: 254 nm; RT1 (min): 8 to afford Example 27 (12 mg, 25%) as a white solid. m/z (ESI+ve ion)=402.20 [M+H]⁺. ¹H NMR (400 MHz, Methanol-d₄) δ 8.89 (s, 1H), 8.23-8.20 (m, 2H), 7.86-7.81 (m, 2H), 7.63-7.56 (m, 3H), 7.16-7.10 (m, 2H), 4.78-4.45 (m, 2H), 4.25-4.22 (m, 2H).

### Example 29. 3-[4-(4-Fluorophenyl)-5-{2-[4-(4-methylpiperazin-1-yl)phenyl]-1H-pyrrolo[2,3-b]pyridin-4-yl}-1,3-oxazol-2-yl]propan-1-ol

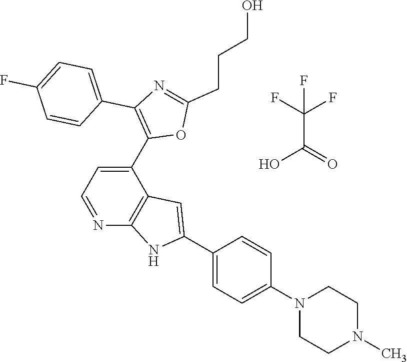 [SMILES: CN1CCN(c2ccc(-c3cc4c(-c5oc(CCCO)nc5-c5ccc(F)cc5)ccnc4[nH]3)cc2)CC1 ; O=C(O)C(F)(F)F]

### Step 1. 1-{4-[1-(Benzenesulfonyl)-4-bromopyrrolo[2,3-b]pyridin-2-yl]phenyl}-4-methylpiperazine

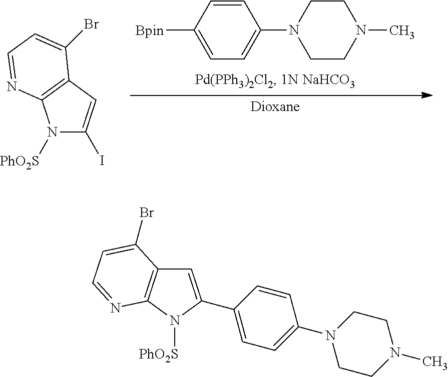

To a stirred solution of 1-(benzenesulfonyl)-4-bromo-2-iodopyrrolo[2,3-b]pyridine (Intermediate D, 1 g, 2.159 mmol, 1 equiv), Pd(PPh₃)₂Cl₂ (0.15 g, 0.22 mmol, 0.1 equiv) and 1N NaHCO₃ (4.3 mL, 4.32 mmol, 2.0 equiv) in 1,4-dioxane (13.2 mL) was added 1-methyl-4-[4-(4,4,5,5-tetramethyl-1,3,2-dioxaborolan-2-yl)phenyl]piperazine (0.65 g, 2.16 mmol, 1.0 equiv) at room temperature under nitrogen atmosphere. The resulting mixture was stirred for 2 h at 80° C. under nitrogen atmosphere and then cooled to room temperature. The resulting mixture was extracted with EA (3×50 mL). The combined organic layers were washed with brine (2×50 mL), dried over anhydrous Na₂SO₄. After filtration, the filtrate was concentrated under reduced pressure. The residue was purified by silica gel column chromatography, eluted with PE:EA (1:1) to afford the title compound (720 mg, 65%) as a yellow oil. m/z (ESI, +ve ion)=511.15, 513.15 [M+H]⁺.

### Step 2. 1-{4-[1-(Benzenesulfonyl)-4-(4,4,5,5-tetramethyl-1,3,2-dioxaborolan-2-yl)pyrrolo[2,3-b]pyridin-2-yl]phenyl}-4-methylpiperazine

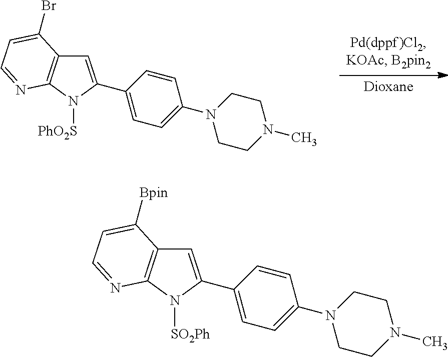

To a stirred mixture of 1-{4-[1-(benzenesulfonyl)-4-bromopyrrolo[2,3-b]pyridin-2-yl]phenyl}-4-methylpiperazine (200 mg, 0.391 mmol, 1.0 equiv) and bis(pinacolato)diboron (148.96 mg, 0.587 mmol, 1.5 equiv) in dioxane (10 mL) were added Pd(dppf)Cl₂·CH₂Cl₂ (31.86 mg, 0.039 mmol, 0.1 equiv) and KOAc (76.76 mg, 0.782 mmol, 2.0 equiv) at room temperature under nitrogen atmosphere. The resulting mixture was stirred for 5 h at 80° C. under nitrogen atmosphere then concentrated under reduced pressure to afford crude the title compound (200 mg, 92%) as a yellow oil. m/z (ESI, +ve ion)=477.15 [M+H]⁺.

### Step 3. 3-[4-(4-Fluorophenyl)-5-{2-[4-(4-methylpiperazin-1-yl)phenyl]-1H-pyrrolo[2,3-b]pyridin-4-yl}-1,2,3-triazol-2-yl]propan-1-ol

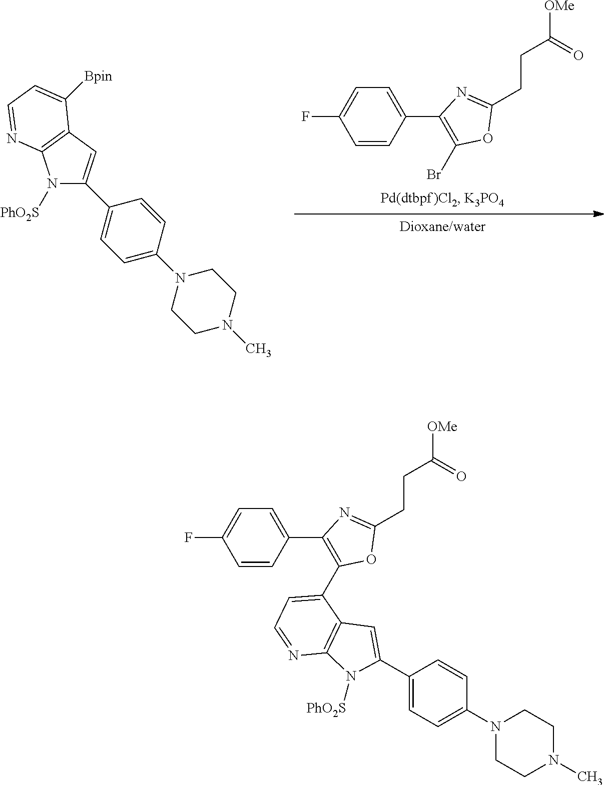

To a stirred mixture of methyl 3-[5-bromo-4-(4-fluorophenyl)-1,3-oxazol-2-yl]propanoate (Intermediate O, 82.25 mg, 0.251 mmol, 1.0 equiv) and 1-{4-[1-(benzenesulfonyl)-4-(4,4,5,5-tetramethyl-1,3,2-dioxaborolan-2-yl)pyrrolo[2,3-b]pyridin-2-yl]phenyl}-4-methylpiperazine (140 mg, 0.25 mmol, 1.0 equiv) in dioxane (4 mL) and water (1 mL) were added Pd(DtBPF)Cl₂ (16 mg, 0.025 mmol, 0.1 equiv) and K₃PO₄ (106 mg, 0.50 mmol, 2.0 equiv) at room temperature under nitrogen atmosphere. The resulting mixture was stirred for 2 h at 80° C. under nitrogen atmosphere then concentrated under reduced pressure. The residue was purified by silica gel column chromatography, eluted with PE:EA (1:1) to afford the title compound (80 mg, 47%) as a yellow solid. m/z (ESI, +ve ion)=680.25 [M+H]⁺.

### Step 4. 3-[4-(4-Fluorophenyl)-5-{2-[4-(4-methylpiperazin-1-yl)phenyl]-1H-pyrrolo[2,3-b]pyridin-4-yl}-1,3-oxazol-2-yl]propan-1-ol

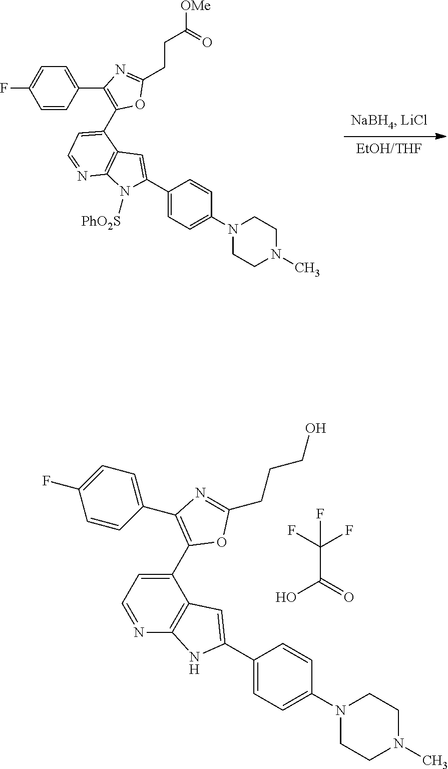

To a stirred mixture of methyl 3-{5-[1-(benzenesulfonyl)-2-[4-(4-methylpiperazin-1-yl)phenyl]pyrrolo[2,3-b]pyridin-4-yl]-4-(4-fluorophenyl)-1,3-oxazol-2-yl}propanoate (80 mg, 0.118 mmol, 1 equiv) and LiCl (0.50 mg, 0.012 mmol, 0.1 equiv) in EtOH (1 mL) and THF (1 mL) were added NaBH₄ (22.26 mg, 0.590 mmol, 5 equiv) in portions at 0° C. under nitrogen atmosphere. The resulting mixture was stirred for 16 h at room temperature under nitrogen atmosphere then concentrated under vacuum. The crude was purified by prep-HPLC with the following conditions: Column: Xselect CSH C18 OBD Column 30×150 mm 5 μm, Mobile Phase A: ACN, Mobile Phase B: water (0.05% TFA); Flow rate: 60 mL/min; Gradient: 10% B to 30% B in 8 min, 30% B; Wavelength: 254/220 nm; RT1 (min): 7.96 to afford Example 29 trifluoroacetate salt (10 mg, 13%) as a yellow solid. m/z (ESI, +ve ion)=512.25 [M+H]⁺. ¹H-NMR (400 MHz, Methanol-d₄) δ 8.14 (s, 1H), 7.68 (d, J=8.8 Hz, 2H), 7.62-7.59 (m, 2H), 7.22-7.11 (m, 5H), 6.43 (s, 1H), 4.86 (s, 3H), 3.87-3.81 (m, 2H), 3.78-3.59 (m, 5H), 3.18-3.07 (m, 2H), 2.99 (s, 3H), 2.19-2.12 (m, 2H).

### Example 31. 4-[3-(4-Fluorophenyl)-1-(oxetan-3-yl)-1H-pyrazol-4-yl]-6-phenylfuro[2,3-d]pyrimidine

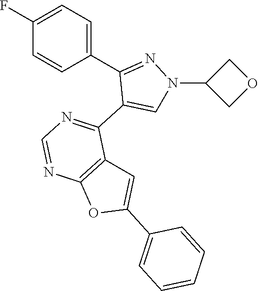 [SMILES: Fc1ccc(-c2nn(C3COC3)cc2-c2ncnc3oc(-c4ccccc4)cc23)cc1]

### Step 1. 4-[3-(4-Fluorophenyl)-1-(oxetan-3-yl)-1H-pyrazol-4-yl]-6-phenylfuro[2,3-d]pyrimidine

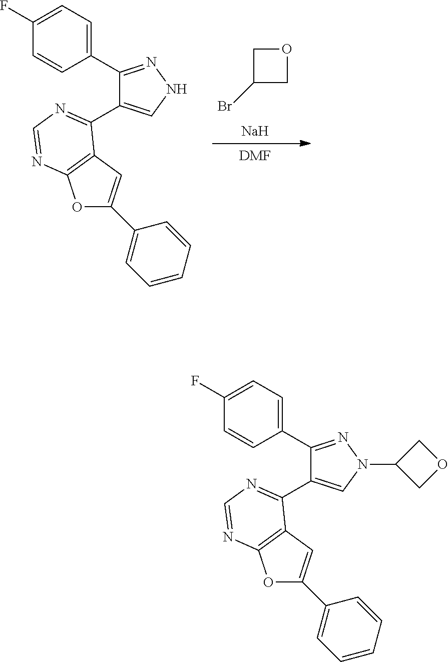

Sodium hydride (16.8 mg, 421 μmol, 1.5 equiv., 60% in dispersion in mineral oil) was added to a mixture of 4-[3-(4-fluorophenyl)-1H-pyrazol-4-yl]-6-phenylfuro[2,3-d]pyrimidine (Intermediate N, 100 mg, 281 μmol) in DMF (2.81 mL) at 0° C. The mixture was stirred for 30 min at 0° C. then 3-bromooxetane (23.3 μL, 267 μmol, 0.95 equiv.) was added. The mixture was stirred for 2 days at room temperature and diluted with water (10 mL). The mixture was extracted with EtOAc (3×10 mL). The combined organic layers were washed with brine, then dried (Na₂SO₄), filtered and concentrated. The residue was purified by column chromatography (EtOAc in hexanes, 0-70%, a gradient elution) then by using reverse phase chromatography (C18 column, MeCN in 10 mM ammonium formate in water, 50-100%, a gradient elution) to provide the title compound (33.0 mg, 26%) as a solid. ¹H NMR (400 MHz, DMSO-d₆) δ 8.78 (s, 1H), 8.75 (s, 1H), 7.96-7.90 (m, 2H), 7.65 (dd, J=8.9, 5.6 Hz, 2H), 7.57 (t, J=7.4 Hz, 2H), 7.53-7.47 (m, 2H), 7.21 (t, J=9.0 Hz, 2H), 5.80-5.69 (m, 1H), 5.09 (t, J=6.6 Hz, 2H), 5.02 (t, J=7.4 Hz, 2H). m/z (ESI, +ve ion)=413.2 [M+H]⁺.

### Example 33. 3-[3-(4-Fluorophenyl)-4-{6-[4-(4-methylpiperazin-1-yl)phenyl]furo[2,3-d]pyrimidin-4-yl}-1H-pyrazol-1-yl]propan-1-ol

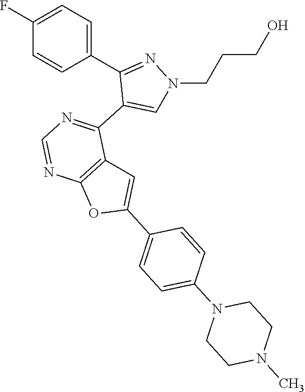 [SMILES: CN1CCN(c2ccc(-c3cc4c(-c5cn(CCCO)nc5-c5ccc(F)cc5)ncnc4o3)cc2)CC1]

### Step 1. 2-Amino-5-(4-bromophenyl)furan-3-carbonitrile

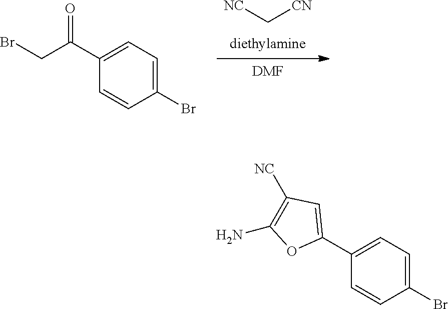

Diethylamine (8.08 mL, 77.6 mmol, 2.2 equiv.) was added dropwise over 30 minutes into a mixture of 2,4′-dibromoacetophenone (10.0 g, 35.3 mmol) and malononitrile (3.19 g, 45.8 mmol, 1.3 equiv.) in DMF (20 mL) at 0° C. The reaction mixture was stirred at 0° C. for additional 15 minutes upon completion of addition, then slowly warmed to room temperature over 16 hours. The mixture was diluted with water (50 mL), stirred vigorously, then the resulting precipitate was collected by suction filtration. The resulting solid was washed repeatedly with water, hexane, and cold ethanol subsequently to provide the title compound (3.85 g, 42%) as a solid. m/z (ESI, −ve ion)=261.0, 263.0 [M−H]⁻.

### Step 2. 6-(4-Bromophenyl)furo[2,3-d]pyrimidin-4 (3H)-one

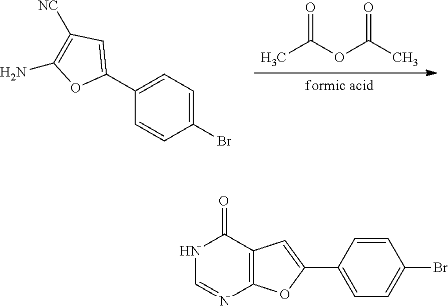

A mixture of 2-amino-5-(4-bromophenyl)furan-3-carbonitrile (720 mg, 2.74 mmol) and formic acid (8.43 mL, 219 mmol, 80.0 equiv.) was cooled to 0° C., and acetic anhydride (7.92 mL, 82.1 mmol, 30 equiv.) was added dropwise. The reaction mixture was stirred for 1 hour at 0° C., the slowly warmed to room temperature and then to 100° C. for 16 hours. The reaction mixture was cooled to room temperature and concentrated under reduced pressure to ˜⅓ of the original volume, then water (50 mL) was added. The resulting precipitate was collected by suction filtration. The residue was triturated in hot MeOH. The mixture was cooled to room temperature and the resulting precipitate was collected by suction filtration and washed with MeOH to provide the title compound (0.383 g, 48%) as a solid. ¹H NMR (400 MHz, DMSO-d₆) δ 12.69 (br s, 1H), 8.16 (d, J=3.8 Hz, 1H), 7.85-7.75 (m, 2H), 7.72-7.63 (m, 2H), 7.56 (s, 1H). m/z (ESI, −ve ion)=289.0 [M−H]⁻.

### Step 3. 6-(4-(4-Methylpiperazin-1-yl)phenyl)furo[2,3-d]pyrimidin-4 (3H)-one

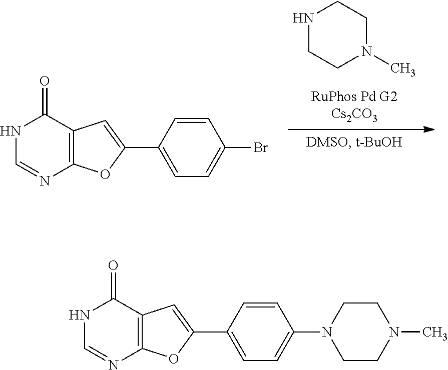

Cesium carbonate (824 mg, 2.50 mmol, 3.0 equiv.) and 6-(4-bromophenyl)furo[2,3-d]pyrimidin-4 (3H)-one (243 mg, 835 μmol) were suspended in a mixture of DMSO (2.47 mL) and t-butanol (309 μL). The mixture was degassed with nitrogen for 5 min and 1-methylpiperazine (935 μL, 8.35 mmol, 10 equiv.) and RuPhos Pd G2 (132 mg, 167 μmol, 0.2 equiv.) were added. The mixture was further degassed for 3 minutes then heated at 100° C. for 4 h. The mixture cooled to room temperature, diluted with EtOAc, and filtered. The filtrate was concentrated under reduced pressure. The residue was purified by reverse phase chromatography (MeCN in 10 mM ammonium formate in water, 5-30%, a gradient elution) to provide the title compound 33c (183 mg, 71%) as a solid. ¹H NMR (400 MHz, DMSO-d₆) δ 8.23 (br s, 1H), 8.08 (s, 1H), 7.68 (d, J=8.9 Hz, 2H), 7.19 (s, 1H), 7.02 (d, J=9.0 Hz, 2H), 3.26-3.18 (m, 4H), 2.48-2.43 (m, 4H), 2.23 (s, 3H). m/z (ESI, +ve ion)=311.2 [M+H]⁺.

### Step 4. 4-Bromo-6-(4-(4-methylpiperazin-1-yl)phenyl)furo[2,3-d]pyrimidine

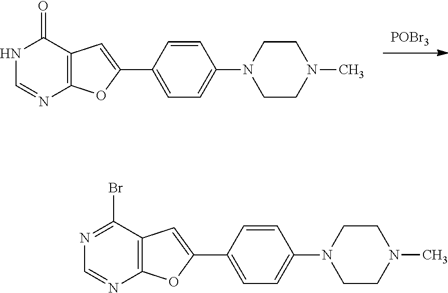 [SMILES: CN1CCN(c2ccc(-c3cc4c(=O)[nH]cnc4o3)cc2)CC1 ; "POBr3" ; CN1CCN(c2ccc(-c3cc4c(Br)ncnc4o3)cc2)CC1]

6-(4-(4-Methylpiperazin-1-yl)phenyl)furo[2,3-d]pyrimidin-4 (3H)-one (180 mg, 580 μmol) and phosphorus(V) oxybromide (3.50 g, 11.6 mmol, 20 equiv.) was heated at 65° C. for 16 hours. The reaction mixture was poured into ice/water mixture (10 mL) and diluted with EtOAc (10 mL). The pH of the aqueous layer was carefully adjusted to ˜ 7-8 using 15% w/w NaOH solution in water. The layers were separated, and the aqueous layer was extracted with additional EtOAc (2×25 mL). The combined organic layers were dried over Na₂SO₄, filtered, and concentrated under reduced pressure. The residue was purified reverse phase chromatography (C18 column, MeCN in 10 mM ammonium formate in water, 5-40%, a gradient elution) to provide the title compound (63.8 mg, 29%) as a solid. ¹H NMR (400 MHz, DMSO-d₆) δ 8.67 (s, 1H), 7.87 (d, J=8.9 Hz, 2H), 7.35 (s, 1H), 7.07 (d, J=9.0 Hz, 2H), 3.33-3.27 (m, 4H), 2.47-2.42 (m, 4H), 2.23 (s, 3H). m/z (ESI, +ve ion)=373.1, 375.1 [M+H]⁺.

### Step 5. 4-(3-(4-Fluorophenyl)-1-(3-((triisopropylsilyl)oxy)propyl)-1H-pyrazol-4-yl)-6-(4-(4-methylpiperazin-1-yl)phenyl)furo[2,3-d]pyrimidine

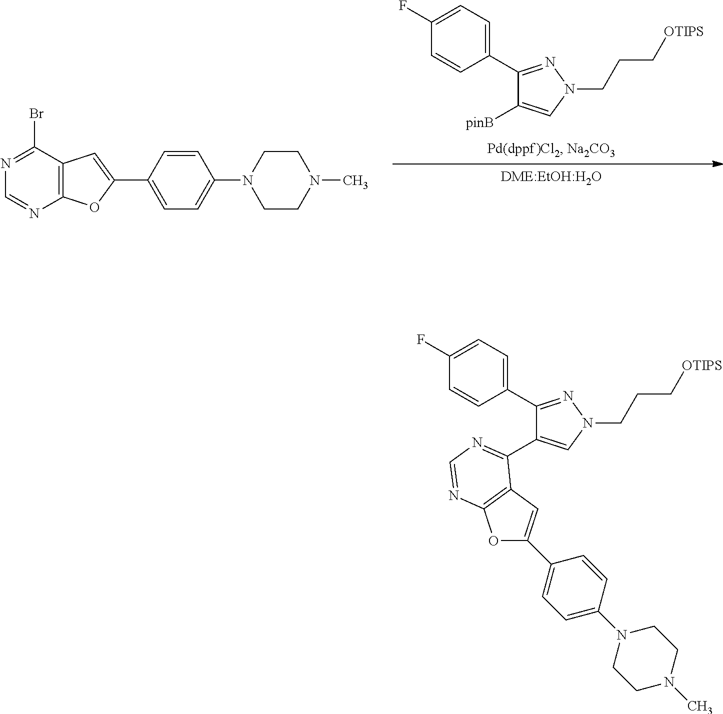

1,1′-Bis(diphenylphosphino)ferrocene dichloropalladium (II) (12.4 mg, 16.1 μmol, 0.1 equiv) was added to a degassed mixture of 4-bromo-6-(4-(4-methylpiperazin-1-yl)phenyl)furo[2,3-d]pyrimidine (60.0 mg, 161 μmol), 3-(4-fluorophenyl)-4-(4,4,5,5-tetramethyl-1,3,2-dioxaborolan-2-yl)-1-(3-((triisopropylsilyl)oxy)propyl)-1H-pyrazole (Intermediate F, 121 mg, 241 μmol, 1.5 equiv.) and sodium carbonate (51.6 mg, 482 μmol, 3.0 equiv) in a mixture of DME (2.31 mL), EtOH (867 μL) and water (290 μL). The mixture was further degassed for 3 min then heated at 120° C. for 1 hour in a microwave reactor. After cooling to room temperature the mixture was diluted with EtOAc and filtered. The filtrate was concentrated under reduced pressure, and the residue was purified reverse phase chromatography (C18 column, MeCN in 10 mM ammonium formate in water, 10-100%, a gradient elution). The material was further purified by column chromatography (MeOH in DCM, 1-10%, a gradient elution) to provide the title compound (108 mg, quant.) as an oil. m/z (ESI, +ve ion)=669.7 [M+H]⁺.

### Step 6. 3-[3-(4-Fluorophenyl)-4-{6-[4-(4-methylpiperazin-1-yl)phenyl]furo[2,3-d]pyrimidin-4-yl}-1H-pyrazol-1-yl]propan-1-ol

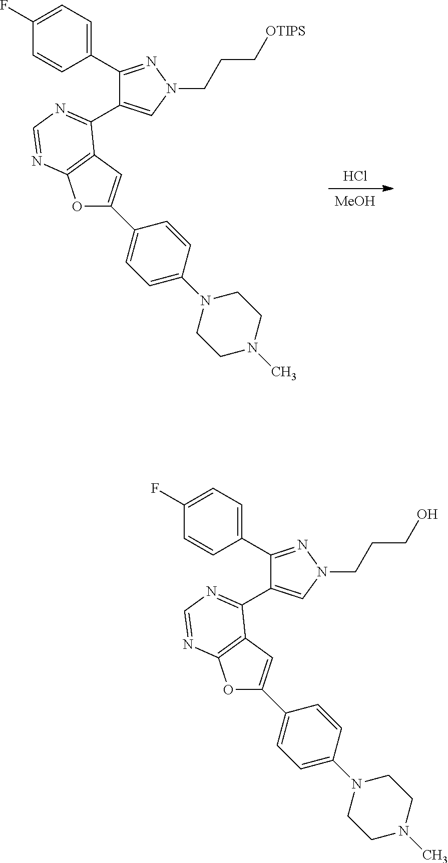

Hydrochloric acid (804 μL, 804 μmol, 5.0 equiv, 1 M in water) was added dropwise into a mixture of 4-(3-(4-fluorophenyl)-1-(3-((triisopropylsilyl)oxy)propyl)-1H-pyrazol-4-yl)-6-(4-(4-methylpiperazin-1-yl)phenyl)furo[2,3-d]pyrimidine (108 mg, 161 μmol) in a mixture of THF (613 μL) and water (289 μL, 1.8 equiv). The resulting mixture was stirred at room temperature for 16 h. The pH of the reaction mixture was adjusted to 7-8 using 15% w/w NaOH solution in water, then EtOAc (10 mL) was added. The aqueous layer was further extracted with EtOAc (5×20 mL) and the combined organic layers were washed with brine (10 mL), dried over Na₂SO₄, filtered and concentrated. The residue was purified by reverse phase chromatography (C18 column, MeCN in 10 mM ammonium formate in water, 5-50%, a gradient elution) to provide the title compound (13.6 mg, 17%) as a solid. ¹H NMR (400 MHz, DMSO-d₆) δ 8.68 (s, 1H), 8.59 (s, 1H), 7.73 (d, J=8.8 Hz, 2H), 7.59 (dd, J=8.6, 5.7 Hz, 2H), 7.18 (app t, J=8.9 Hz, 2H), 7.13 (s, 1H), 7.08 (d, J=9.0 Hz, 2H), 4.33 (t, J=7.1 Hz, 2H), 3.50 (t, J=6.0 Hz, 2H), 3.32-3.25 (m, 4H), 2.54 (s, 1H), 2.47-2.42 (m, 4H), 2.23 (s, 3H), 2.07 (p, J=6.4 Hz, 2H). m/z (ESI, +ve ion)=513.5 [M+H]⁺.

### Example 35. 3-[4-(4-Fluorophenyl)-5-(6-phenylfuro[2,3-d]pyrimidin-4-yl)-1,3-oxazol-2-yl]propan-1-ol

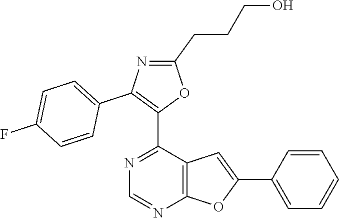 [SMILES: OCCCc1nc(-c2ccc(F)cc2)c(-c2ncnc3oc(-c4ccccc4)cc23)o1]

### Step 1. 3-[(Triisopropylsilyl)oxy]propan-1-ol

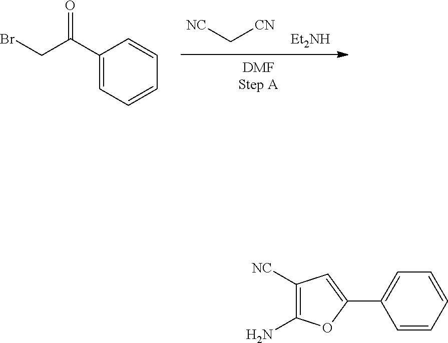

To a stirred mixture of phenacyl bromide (20 g, 100.5 mmol, 1 equiv) and malononitrile (8.63 g, 130.6 mmol, 1.3 equiv) in DMF (43 mL) was added diethylamine (16.17 g, 221 mmol, 2.2 equiv) dropwise at 0° C. under nitrogen atmosphere. The resulting mixture was stirred for 1 h at room temperature under nitrogen atmosphere then diluted with water (170 mL) and stirred for 20 min at room temperature. The precipitated solids were collected by filtration and washed with water (3×50 mL). The residue was dissolved in EtOH (300 mL). The resulting mixture was stirred for 5 min at 85° C. then cooled down to room temperature. The precipitated solids were collected by filtration and washed with EtOH (3×60 mL). The residue was dissolved in NH₃ (30% aq., 250 mL). The resulting mixture was extracted with CHCl₃ (3×200 mL). The combined organic layers were washed with brine (300 mL), dried over anhydrous Na₂SO₄. After filtration, the filtrate was concentrated under reduced pressure to afford the title compound (4 g, 22%) as a light brown solid. m/z (ESI, +ve ion)=185.10 [M+H]⁺.

### Step 2. 6-Phenyl-3H-furo[2,3-d]pyrimidin-4-one

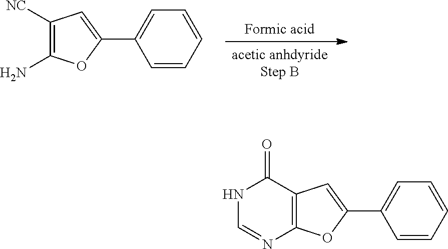 [SMILES: N#Cc1cc(-c2ccccc2)oc1N ; "Formic acid" ; "acetic anhdyrideStep B" ; O=c1[nH]cnc2oc(-c3ccccc3)cc12]

A solution of acetic anhydride (36.03 g, 352 mmol, 25 equiv) and formic acid (19.49 g, 424 mmol, 30 equiv) was stirred for 30 min at 0° C. under nitrogen atmosphere. To the above mixture was added 2-amino-5-phenylfuran-3-carbonitrile (2.6 g, 14.1 mmol, 1 equiv) in portions over 5 min at 30° C. The resulting mixture was stirred for additional 3 h at 80° C. The resulting mixture was stirred for overnight at 130° C. under nitrogen atmosphere then cooled down to room temperature. The resulting mixture was diluted with diisopropyl ether (50 mL) and cooled down to 0° C. The precipitated solids were collected by filtration and washed with diisopropyl ether (3×30 mL) to afford the title compound (2.5 g, 84%) as a brown solid. m/z (ESI, +ve ion)=213.05 [M+H]⁺.

### Step 3. 4-Bromo-6-phenylfuro[2,3-d]pyrimidine

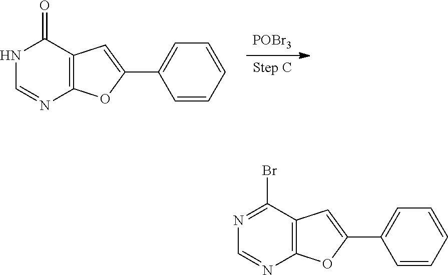 [SMILES: O=c1[nH]cnc2oc(-c3ccccc3)cc12 ; "POBr3" ; "Step C" ; Brc1ncnc2oc(-c3ccccc3)cc12]

A mixture of 6-phenyl-3H-furo[2,3-d]pyrimidin-4-one (400 mg, 1.89 mmol, 1 equiv) and POBr₃ (7.03 g, 24.5 mmol, 13 equiv) was stirred for 3 h at 65° C. The reaction was quenched by the addition of sat. NaOH (aq., 200 mL) at 0° C. The resulting mixture was extracted with EtOAc (3×150 mL). The combined organic layers were washed with brine (2×100 mL), dried over anhydrous Na₂SO₄. After filtration, the filtrate was concentrated under reduced pressure. The residue was purified by silica gel column chromatography, eluted with PE:EA (4:1) to afford the title compound (425 mg, 82%) as a brown solid. m/z (ESI, +ve ion)=274.85, 276.85 [M+H]⁺.

### Step 4. 4-Bromo-6-phenylfuro[2,3-d]pyrimidine

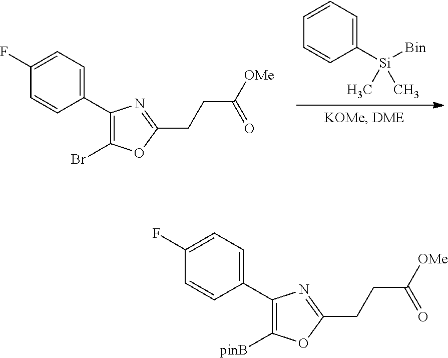

To a stirred mixture of potassium methoxide (718 mg, 10.2 mmol, 2.8 equiv) in DME (12 mL) was added dimethyl(phenyl)(4,4,5,5-tetramethyl-1,3,2-dioxaborolan-2-yl)silane (3.36 g, 12.8 mmol, 3.5 equiv) at room temperature under nitrogen atmosphere. The resulting mixture was stirred for 20 min at 30° C. under nitrogen atmosphere. To the above mixture was added methyl 3-[5-bromo-4-(4-fluorophenyl)-1,3-oxazol-2-yl]propanoate (Intermediate 0, 1.2 g, 3.66 mmol, 1 equiv) in portions at 30° C. The resulting mixture was stirred for additional 1.5 h at 30° C. then diluted with DME (15 mL). The resulting mixture was filtered, the filter cake was washed with DME (3×10 mL). The filtrate was concentrated under reduced pressure to afford the title compound (1.2 g, 88%) as an orange solid. m/z (ESI, +ve ion)=376.20 [M+H]⁺.

### Step 5. Methyl 3-[4-(4-fluorophenyl)-5-{6-phenylfuro[2,3-d]pyrimidin-4-yl}-1,3-oxazol-2-yl]propanoate

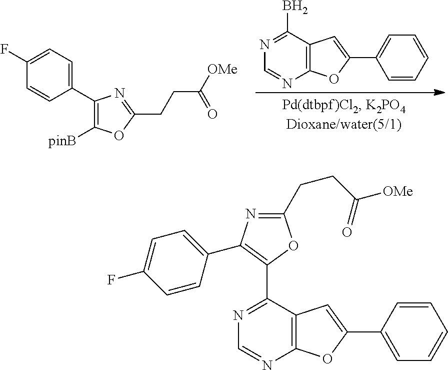

To a stirred mixture of methyl 3-[4-(4-fluorophenyl)-5-(4,4,5,5-tetramethyl-1,3,2-dioxaborolan-2-yl)-1,3-oxazol-2-yl]propanoate (424 mg, 1.13 mmol, 1.5 equiv) and 4-bromo-6-phenylfuro[2,3-d]pyrimidine (Intermediate I, 207 mg, 0.75 mmol, 1.00 equiv) in 1,4-dioxane (2 mL) and H₂O (0.4 mL) were added K₃PO₄ (319 mg, 1.50 mmol, 2 equiv) and Pd(DtBPF)Cl₂ (98.1 mg, 0.15 mmol, 0.2 equiv) at room temperature under nitrogen atmosphere. The resulting mixture was stirred for 2 h at 90° C. under nitrogen atmosphere then cooled down to room temperature. The reaction was quenched by the addition of water (10 mL) at room temperature. The resulting mixture was extracted with EtOAc (3×20 mL). The combined organic layers were washed with brine (2×20 mL), dried over anhydrous Na₂SO₄. After filtration, the filtrate was concentrated under reduced pressure. The residue was purified by silica gel column chromatography, eluted with PE:EA (4:1) to afford the title compound (150 mg, 45%) as a yellow solid. m/z (ESI, +ve ion)=444.20 [M+H]⁺.

### Step 6. 3-[4-(4-Fluorophenyl)-5-{6-phenylfuro[2,3-d]pyrimidin-4-yl}-1,3-oxazol-2-yl]propan-1-ol

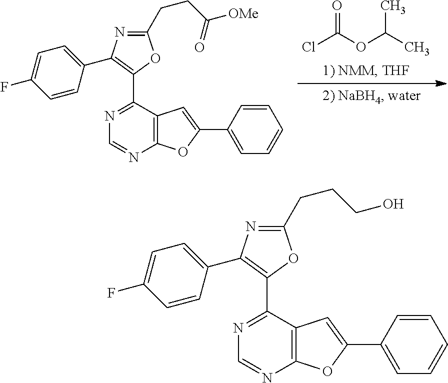

To a stirred mixture of 3-[4-(4-fluorophenyl)-5-{6-phenylfuro[2,3-d]pyrimidin-4-yl}-1,3-oxazol-2-yl]propanoic acid (75 mg, 0.18 mmol, 1 equiv) and NMM (26.50 mg, 0.262 mmol, 1.5 equiv) in THF (1.4 mL) was added chloro(propan-2-yloxy)methanone (24 mg, 0.19 mmol, 1.1 equiv) dropwise at 0° C. under nitrogen atmosphere. The resulting mixture was stirred for 1 h at 0° C. under nitrogen atmosphere. To the above mixture was added H₂O (0.7 mL) dropwise at 0° C. and NaBH₄ (13.9 mg, 0.37 mmol, 2.1 equiv) was added in portions over 1 min at 0° C. The resulting mixture was stirred for additional 1 h at 0° C. The reaction was quenched by the addition of water (10 mL) at room temperature. The resulting mixture was extracted with EtOAc (3×15 mL). The combined organic layers were washed with brine (40 mL), dried over anhydrous Na₂SO₄. After filtration, the filtrate was concentrated under reduced pressure. The residue was purified by reverse flash chromatography with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 51% B to 61% B in 8 min, 61% B; Wavelength: 254 nm; RT1 (min): 7 to afford Example 35 (25.8 mg, 36%) as a yellow solid. m/z (ESI, +ve ion)=416.15 [M+H]⁺. ¹H-NMR (400 MHz, DMSO-d₆) δ 8.91 (s, 1H), 8.29-8.26 (m, 2H), 8.08 (d, J=7.2 Hz, 2H), 7.82 (s, 1H), 7.62-7.54 (m, 3H), 7.34-7.29 (m, 2H), 4.70-4.68 (m, 1H), 3.63-3.58 (m, 2H), 3.14-3.10 (m, 2H), 2.03-2.08 (m, 2H).

### Example 38. 4-{3-(4-Fluorophenyl)-1-[2-(methanesulfonyl)ethyl]-1H-pyrazol-4-yl}-6-phenylfuro[2,3-d]pyrimidine

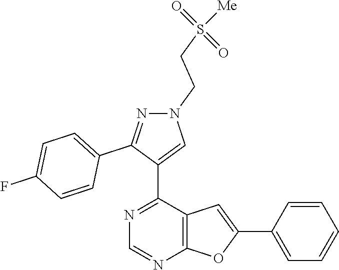 [SMILES: CS(=O)(=O)CCn1cc(-c2ncnc3oc(-c4ccccc4)cc23)c(-c2ccc(F)cc2)n1]

### Step 1. 3-(4-Fluorophenyl)-1-(2-methanesulfonylethyl)-4-{6-phenylfuro[2,3-d]pyrimidin-4-yl}pyrazole

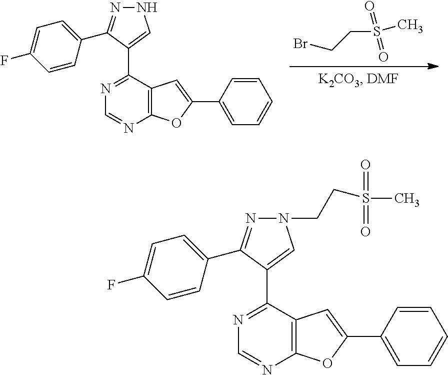

To a stirred solution of 4-[3-(4-Fluorophenyl)-1H-pyrazol-4-yl]-6-phenylfuro[2,3-d]pyrimidine (Intermediate N, 120 mg, 0.34 mmol, 1 equiv) in DMF (3 mL) were added K₂CO₃ (93 mg, 0.67 mmol, 2 equiv) at room temperature under nitrogen atmosphere. The resulting mixture was stirred for 10 min at room temperature under nitrogen atmosphere. To the above mixture was added 1-bromo-2-methanesulfonylethane (95 mg, 0.51 mmol, 1.5 equiv). The resulting mixture was stirred for additional 16 h at room temperature then quenched by the addition of water (10 mL) at 0° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The residue was purified by prep-TLC (PE:EA=4:1) to afford Example 38 (140 mg, 90%) as a white solid. m/z (ESI, +ve ion)=463.20 [M+H]⁺. ¹H-NMR (400 MHz, DMSO-d₆) δ 8.78 (s, 1H), 8.74 (s, 1H), 7.91 (d, J=8.8 Hz, 2H), 7.65-7.62 (m, 2H), 7.61-7.52 (m, 2H), 7.51-7.50 (m, 1H), 7.43 (s, 1H), 7.24-7.19 (m, 2H), 4.75 (t, J=7.2 Hz, 2H), 3.90 (t, J=7.2 Hz, 2H), 3.04 (s, 3H).

### Example 40. 3-[3-(4-Fluorophenyl)-4-(6-phenylfuro[2,3-d]pyrimidin-4-yl)-1H-pyrazol-1-yl]-1λ6-thietane-1,1-dione

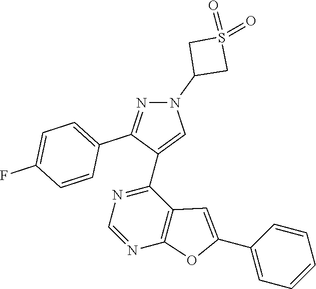 [SMILES: O=S1(=O)CC(n2cc(-c3ncnc4oc(-c5ccccc5)cc34)c(-c3ccc(F)cc3)n2)C1]

### Step 1. 3-[3-(4-Fluorophenyl)-4-(6-phenylfuro[2,3-d]pyrimidin-4-yl)-1H-pyrazol-1-yl]-1λ6-thietane-1,1-dione

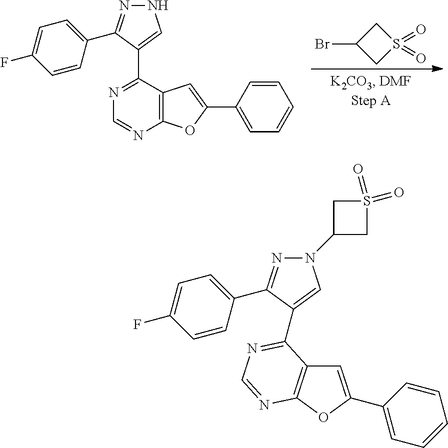 [SMILES: Fc1ccc(-c2n[nH]cc2-c2ncnc3oc(-c4ccccc4)cc23)cc1 ; O=S1(=O)CC(Br)C1 ; O=S1(=O)CC(n2cc(-c3ncnc4oc(-c5ccccc5)cc34)c(-c3ccc(F)cc3)n2)C1]

To a stirred mixture of 4-[3-(4-Fluorophenyl)-1H-pyrazol-4-yl]-6-phenylfuro[2,3-d]pyrimidine (Intermediate N, 200 mg, 0.561 mmol, 1 equiv) and K₂CO₃ (155 mg, 1.12 mmol, 2 equiv) in DMF (2 mL) was added 3-bromo-1,6-thietane-1,1-dione (135.01 mg, 0.73 mmol, 1.3 equiv) at room temperature under nitrogen atmosphere. The resulting mixture was stirred for 2 h at room temperature under nitrogen atmosphere. The reaction was quenched by the addition of water (10 mL) at room temperature. The resulting mixture was extracted with EtOAc (3×20 mL). The combined organic layers were washed with brine (30 mL), dried over anhydrous Na₂SO₄. After filtration, the filtrate was concentrated under reduced pressure. The residue was purified by trituration with TBME (10 mL). The precipitated solids were collected by filtration a to afford Example 40 (150.2 mg, 58%) as a white solid. m/z (ESI, +ve ion)=461.20 [M+H]⁺. ¹H-NMR (400 MHz, Chloroform-d) δ 8.95 (s, 1H), 8.28 (s, 1H), 7.66-7.64 (m, 2H), 7.58-7.54 (m, 2H), 7.48-7.41 (m, 3H), 7.14-7.09 (m, 2H), 6.10 (s, 1H), 5.35-5.27 (m, 1H), 4.98-4.93 (m, 2H), 4.75-4.69 (m, 2H).

### Example 42. (1s, 3s)-3-{[3-(4-Fluorophenyl)-4-(6-phenylfuro[2,3-d]pyrimidin-4-yl)-1H-pyrazol-1-yl]methyl}cyclobutan-1-ol

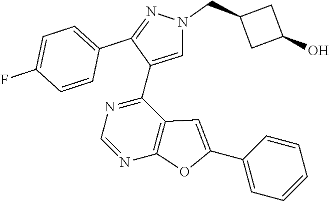 [SMILES: O[C@H]1C[C@@H](Cn2cc(-c3ncnc4oc(-c5ccccc5)cc34)c(-c3ccc(F)cc3)n2)C1]

### Step 1. (1s, 3s)-3-{[3-(4-Fluorophenyl)-4-(6-phenylfuro[2,3-d]pyrimidin-4-yl)-1H-pyrazol-1-yl]methyl}cyclobutan-1-ol

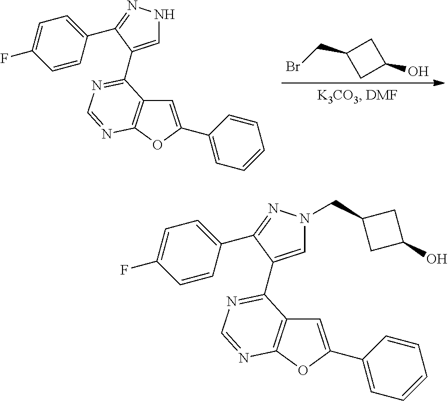 [SMILES: Fc1ccc(-c2n[nH]cc2-c2ncnc3oc(-c4ccccc4)cc23)cc1 ; O[C@H]1C[C@@H](CBr)C1 ; O[C@H]1C[C@@H](Cn2cc(-c3ncnc4oc(-c5ccccc5)cc34)c(-c3ccc(F)cc3)n2)C1]

To a stirred solution of 4-[3-(4-Fluorophenyl)-1H-pyrazol-4-yl]-6-phenylfuro[2,3-d]pyrimidine (Intermediate N, 100 mg, 0.28 mmol, 1 equiv) in DMF (2.5 mL) was added K₂CO₃ (78 mg, 0.56 mmol, 2 equiv) at room temperature under nitrogen atmosphere. The resulting mixture was stirred for 10 min at room temperature under nitrogen atmosphere. To the above mixture was added (1s, 3s)-3-(bromomethyl)cyclobutan-1-ol (46.3 mg, 0.28 mmol, 1 equiv) at room temperature. The resulting mixture was stirred for additional 2 h at 60° C. then quenched by the addition of water (5 mL) at 0° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The crude product was purified by prep-HPLC with the following conditions: Column: Xselect CSH C18 OBD Column 30×150 mm 5 μm, Mobile Phase A: water (0.1% FA), Mobile Phase B: ACN; Flow rate: 60 mL/min; Gradient: 46% B to 46% B in 8 min, 46% B; Wavelength: 254 nm; RT1 (min): 7 to afford Example 42 (22 mg, 18%) as a white solid. m/z (ESI, +ve ion)=441.30 [M+H]⁺. ¹H NMR (400 MHz, Chloroform-d) δ 8.92 (s, 1H), 8.07 (s, 1H), 7.65-7.55 (m, 2H), 7.54-7.47 (m, 2H), 7.45-7.28 (m, 3H), 7.11-7.07 (m, 2H), 6.11-6.07 (m, 1H), 4.32-4.23 (m, 3H), 2.63-2.56 (m, 2H), 2.51-2.43 (m, 1H), 1.87-1.76 (m, 2H).

### Example 43. (1r,3r)-3-{[3-(4-Fluorophenyl)-4-(6-phenylfuro[2,3-d]pyrimidin-4-yl)-1H-pyrazol-1-yl]methyl}cyclobutan-1-ol

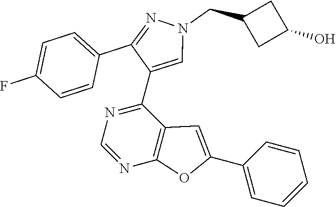

### Step 1. (1r,3r)-3-{[3-(4-Fluorophenyl)-4-(6-phenylfuro[2,3-d]pyrimidin-4-yl)-1H-pyrazol-1-yl]methyl}cyclobutan-1-ol

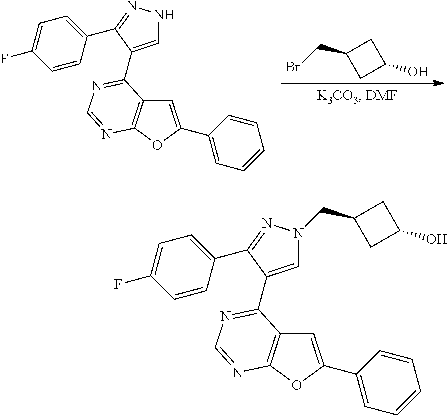 [SMILES: Fc1ccc(-c2n[nH]cc2-c2ncnc3oc(-c4ccccc4)cc23)cc1 ; O[C@H]1C[C@H](CBr)C1 ; O[C@H]1C[C@H](Cn2cc(-c3ncnc4oc(-c5ccccc5)cc34)c(-c3ccc(F)cc3)n2)C1]

To a stirred mixture of 4-(3-(4-fluorophenyl)-1H-pyrazol-4-yl)-6-phenylfuro[2,3-d]pyrimidine (Intermediate N, 100 mg, 0.28 mmol, 1 equiv) and K₂CO₃ (78 mg, 0.56 mmol, 2 equiv) in DMF (2.5 mL, 32.3 mmol, 115.12 equiv) was added (1r,3r)-3-(bromomethyl)cyclobutan-1-ol (46.3 mg, 0.28 mmol, 1 equiv) at room temperature under nitrogen atmosphere. The resulting mixture was stirred for 5 h at room temperature under nitrogen atmosphere. The reaction was quenched by the addition of water (10 mL) at room temperature and extracted with EtOAc (10 mL). The combined organic layers were washed with brine (20 mL), dried over anhydrous Na₂SO₄. After filtration, the filtrate was concentrated under reduced pressure. The residue was purified by prep-HPLC with the following conditions: Column: XBridge Shield RP18 OBD Column, 19×250 mm, 10 μm; Mobile Phase A: water (0.05% FA), Mobile Phase B: ACN; Flow rate: 25 mL/min; Gradient: 52% B to 52% B in 8 min, 52% B; Wavelength: 254 nm; RT1 (min): 8) to afford Example 43 (38.2 mg, 31%) as a white solid and Example 54 (11.8 mg, 10%) as a white solid. m/z (ESI, +ve ion)=441.30 [M+H]⁺. ¹H NMR (400 MHz, DMSO-d₆) δ 8.77 (s, 1H), 8.66 (s, 1H), 7.95-7.87 (m, 2H), 7.65-7.53 (m, 4H), 7.53-7.45 (m, 1H), 7.36 (s, 1H), 7.24-7.14 (m, 2H), 5.06 (d, J=6.0 Hz, 1H), 4.35-4.22 (m, 3H), 2.78-2.69 (m, 1H), 2.20-2.17 (m, 2H), 2.10-1.99 (m, 1H), 2.02-1.97 (m, 1H).

### Example 45. 4-[1-(4,4-Difluorobutyl)-3-(4-fluorophenyl)-1H-pyrazol-4-yl]-6-phenylfuro[2,3-d]pyrimidine

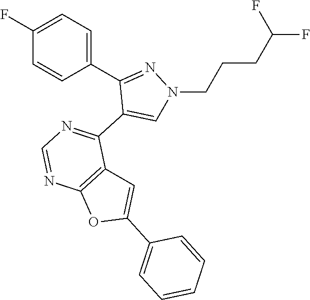 [SMILES: Fc1ccc(-c2nn(CCCC(F)F)cc2-c2ncnc3oc(-c4ccccc4)cc23)cc1]

### Step 1. 4-[1-(4,4-Difluorobutyl)-3-(4-fluorophenyl)-1H-pyrazol-4-yl]-6-phenylfuro[2,3-d]pyrimidine

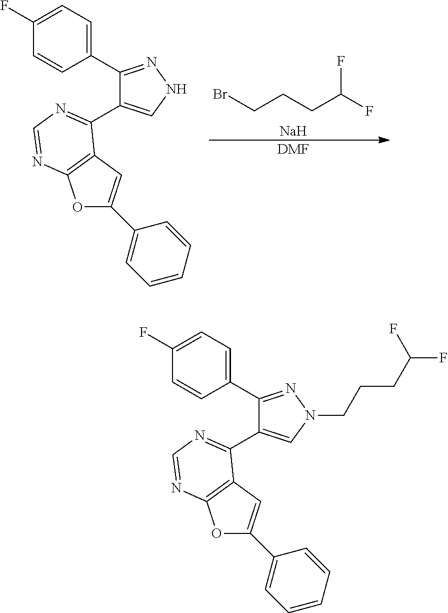

Sodium hydride (16.8 mg, 421 μmol, 1.5 equiv., 60% in dispersion in mineral oil) was added to a mixture of 4-[3-(4-fluorophenyl)-1H-pyrazol-4-yl]-6-phenylfuro[2,3-d]pyrimidine (Intermediate N, 100 mg, 281 μmol) in DMF (2.81 mL) at 0° C. The mixture was stirred for 30 min at 0° C., then 4-bromo-1,1-difluorobutane (71.5 mg, 393 μmol, 1.4 equiv.) was added. The mixture was stirred for 16 h at room temperature and diluted with water (10 mL). The mixture was extracted with EtOAc (3×10 mL). The combined organic layers were washed with brine, dried (Na₂SO₄), filtered and concentrated under reduced pressure. The residue was purified by column chromatography (EtOAc in hexanes, 0-70%, a gradient elution) to provide the title compound (40.0 mg, 32%) as a solid. ¹H NMR (400 MHz, DMSO-d₆) δ 8.77 (s, 1H), 8.66 (s, 1H), 7.91 (dd, J=8.3, 1.2 Hz, 2H), 7.64-7.53 (m, 4H), 7.52-7.45 (m, 1H), 7.41 (s, 1H), 7.19 (t, J=8.9 Hz, 2H), 6.16 (tt, J=56.7, 4.2 Hz, 1H), 4.34 (t, J=6.9 Hz, 2H), 2.12-2.01 (m, 2H), 2.01-1.84 (m, 2H). m/z (ESI, +ve ion)=449.6 [M+H]⁺.

### Example 46. 1-[3-(4-Fluorophenyl)-4-(6-phenylfuro[2,3-d]pyrimidin-4-yl)-1H-pyrazol-1-yl]-2-methylpropan-2-ol

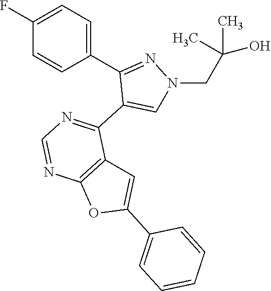 [SMILES: CC(C)(O)Cn1cc(-c2ncnc3oc(-c4ccccc4)cc23)c(-c2ccc(F)cc2)n1]

### Step 1. 1-[3-(4-Fluorophenyl)-4-(6-phenylfuro[2,3-d]pyrimidin-4-yl)-1H-pyrazol-1-yl]-2-methylpropan-2-ol

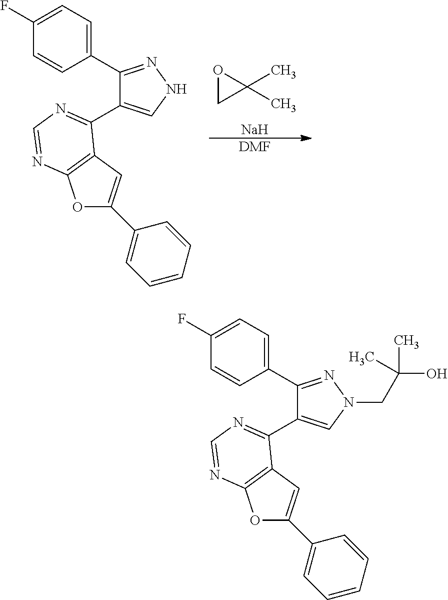

Sodium hydride (16.8 mg, 421 μmol, 1.5 equiv., 60% in dispersion in mineral oil) was added to a mixture of 4-[3-(4-fluorophenyl)-1H-pyrazol-4-yl]-6-phenylfuro[2,3-d]pyrimidine (Intermediate N, 100 mg, 281 μmol) in DMF (2.81 mL) at 0° C. The mixture was stirred for 30 min at 0° C. then 1,2-epoxy-2-methylpropane (51.5 μL, 561 μmol, 2.0 equiv.) was added. The mixture was stirred for 16 hours at 80° C. and cooled to room temperature. The mixture was diluted with water (20 mL) and extracted with EtOAc (3×10 mL). The combined organic layers were washed with water, brine, dried over Na₂SO₄, filtered then concentrated under reduced pressure. The residue was purified by column chromatography (EtOAc in hexanes, 0-100%, a gradient elution) to provide the title compound (46.0 mg, 38%) as a solid. ¹H NMR (400 MHz, DMSO-d₆) δ 8.78 (s, 1H), 8.52 (s, 1H), 7.92-7.86 (m, 2H), 7.64-7.53 (m, 4H), 7.50 (d, J=7.2 Hz, 1H), 7.30 (s, 1H), 7.19 (t, J=8.9 Hz, 2H), 4.86 (s, 1H), 4.20 (s, 2H), 1.20 (s, 6H). m/z (ESI, +ve ion)=429.3 [M+H]⁺.

### Example 47. 4-{3-(4-Fluorophenyl)-1-[(oxetan-3-yl)methyl]-1H-pyrazol-4-yl}-6-phenylfuro[2,3-d]pyrimidine

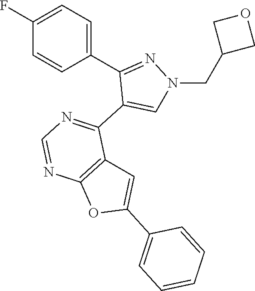 [SMILES: Fc1ccc(-c2nn(CC3COC3)cc2-c2ncnc3oc(-c4ccccc4)cc23)cc1]

### Step 1. 4-{3-(4-Fluorophenyl)-1-[(oxetan-3-yl)methyl]-1H-pyrazol-4-yl}-6-phenylfuro[2,3-d]pyrimidine

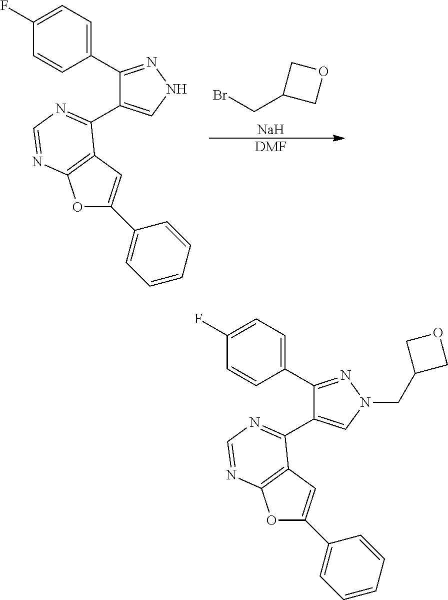

Sodium hydride (16.8 mg, 421 μmol, 1.5 equiv., 60% in dispersion in mineral oil) was added into a mixture of 4-[3-(4-fluorophenyl)-1H-pyrazol-4-yl]-6-phenylfuro[2,3-d]pyrimidine (Intermediate N, 100 mg, 281 μmol) in DMF (2.81 mL) at 0° C. The mixture was stirred for 30 minutes at 0° C. then 3-(bromomethyl)oxetane (50.8 mg, 323 μmol, 1.15 equiv.) was added. The mixture was stirred for 16 hours at room temperature and diluted with water (10 mL). The mixture was extracted with EtOAc (3×10 mL). The combined organic layers were washed with brine, dried over Na₂SO₄, filtered then concentrated. The residue was purified by column chromatography (MeOH in DCM, 0-5%, a gradient elution) to provide the title compound (40.0 mg, 33%) as a solid. ¹H NMR (400 MHz, DMSO-d₆) δ 8.77 (s, 1H), 8.68 (s, 1H), 7.94-7.89 (m, 2H), 7.62-7.53 (m, 4H), 7.53-7.46 (m, 1H), 7.40 (s, 1H), 7.22-7.15 (m, 2H), 4.73 (dd, J=7.8, 6.2 Hz, 2H), 4.59 (d, J=7.4 Hz, 2H), 4.54 (apparent t, J=6.1 Hz, 2H), 3.65-3.51 (m, 1H). m/z (ESI, +ve ion)=427.0 [M+H]⁺.

### Example 48. 4-[3-(4-Fluorophenyl)-1-(2H3)methyl-1H-pyrazol-4-yl]-6-phenylfuro[2,3-d]pyrimidine

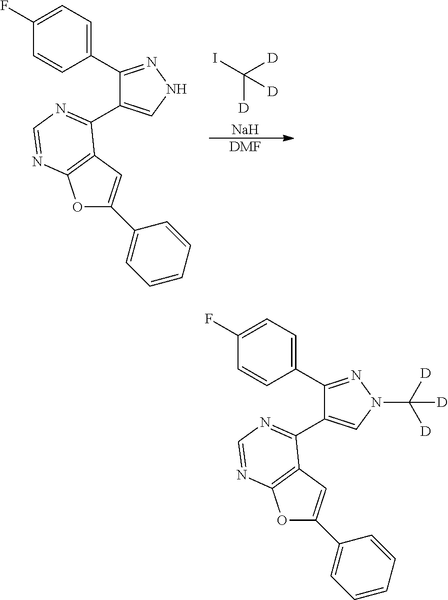

Sodium hydride (16.8 mg, 421 μmol, 1.5 equiv., 60% in dispersion in mineral oil) was added to a mixture of 4-[3-(4-fluorophenyl)-1H-pyrazol-4-yl]-6-phenylfuro[2,3-d]pyrimidine (Intermediate N, 100 mg, 281 μmol) in DMF (2.81 mL) at 0° C. The mixture was stirred for 30 min at 0° C. then iodomethane-d3 (26.3 μL, 421 μmol, 1.5 equiv.) was added. The mixture was stirred for 16 h at room temperature then diluted with water and extracted with EtOAc (3×20 mL). The combined organic layers were washed with brine, dried (Na₂SO₄), filtered then concentrated. The residue was purified by column chromatography (EtOAc in hexanes, 0-70%, a gradient elution) to provide the title compound (51.0 mg, 49%) as a solid. ¹H NMR (400 MHz, DMSO-d₆) δ 8.76 (s, 1H), 8.61 (s, 1H), 7.94-7.89 (m, 2H), 7.63-7.53 (m, 4H), 7.53-7.46 (m, 1H), 7.44 (s, 1H), 7.19 (t, J=8.9 Hz, 2H). m/z (ESI, +ve ion)=374.3 [M+H]⁺.

### Example 52. 3-{2-[3-(4-Fluorophenyl)-4-(6-phenylfuro[2,3-d]pyrimidin-4-yl)-1H-pyrazol-1-yl]ethyl}-1λ6-thietane-1,1-dione

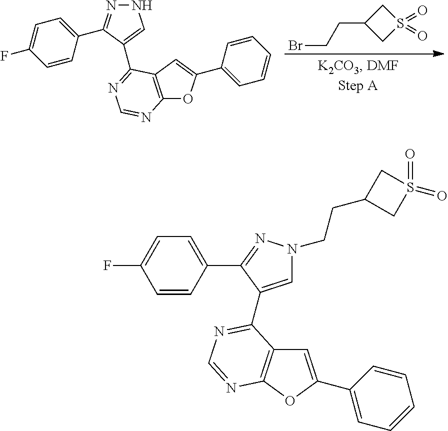

To a stirred solution of 4-[3-(4-Fluorophenyl)-1H-pyrazol-4-yl]-6-phenylfuro[2,3-d]pyrimidine (Intermediate N, 100 mg, 0.281 mmol, 1 equiv) in DMF (3.5 mL) were added K₂CO₃ (77.6 mg, 0.56 mmol, 2 equiv) and 3-(2-bromoethyl)-1,6-thietane-1,1-dione (71.8 mg, 0.34 mmol, 1.2 equiv). The resulting mixture was stirred for additional 16 h at room temperature then quenched by the addition of water (10 mL) at 0° C. The resulting mixture was extracted with EtOAc (3×50 mL). The combined organic layers were washed with brine (3×30 mL), dried over anhydrous Na₂SO₄. After filtration, the filtrate was concentrated under reduced pressure. The crude product was purified by prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 47% B to 51% B in 8 min, 51% B; Wavelength: 254 nm; RT1 (min): 7 to afford Example 52 (60 mg, 44%) as a white solid. m/z (ESI, +ve ion)=489.20 [M+H]⁺. ¹H NMR (400 MHz, Chloroform-d) δ 8.91 (s, 1H), 8.08 (s, 1H), 7.66-7.63 (m, 2H), 7.55-7.53 (m, 2H), 7.52-7.28 (m, 3H), 7.13-7.09 (m, 2H), 6.07 (s, 1H), 4.44-4.26 (m, 4H), 3.86-3.81 (m, 2H), 2.70-2.66 (m, 1H), 2.48-2.43 (m, 2H).

### Example 55. 4-{3-(4-Fluorophenyl)-1-[3-(methanesulfonyl)propyl]-1H-pyrazol-4-yl}-6-phenylfuro[2,3-d]pyrimidine

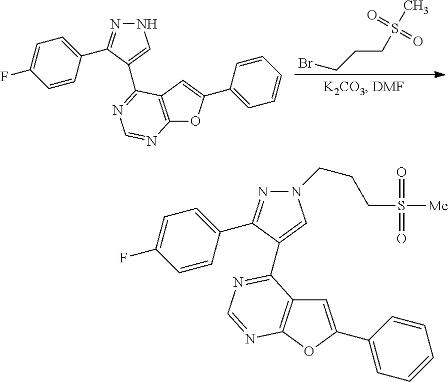

To a stirred solution of 4-[3-(4-Fluorophenyl)-1H-pyrazol-4-yl]-6-phenylfuro[2,3-d]pyrimidine (Intermediate N, 100 mg, 0.28 mmol, 1 equiv) in DMF (2.5 mL) were added K₂CO₃ (78 mg, 0.56 mmol, 2 equiv) at room temperature under nitrogen atmosphere. The resulting mixture was stirred for 10 min at room temperature under nitrogen atmosphere. To the above mixture was added 1-bromo-3-methanesulfonylpropane (84.64 mg, 0.422 mmol, 1.5 equiv) at room temperature. The resulting mixture was stirred for additional 16 h at room temperature. The reaction was quenched by the addition of water (10 mL) at 0° C. The resulting mixture was extracted with EtOAc (3×50 mL). The combined organic layers were washed with brine (2×50 mL), dried over anhydrous Na₂SO₄. After filtration, the filtrate was concentrated under reduced pressure. The crude product was purified by prep-HPLC with the following conditions: Column: XSelect CSH Prep C18 OBD Column, 19×250 mm, 5 μm; Mobile Phase A: water (10 mmol/L NH₄HCO₃), Mobile Phase B: MeOH-HPLC; Flow rate: 25 mL/min; Gradient: 64% B to 64% B in 13 min, 64% B; Wavelength: 254 nm; RT1 (min): 12.4 to afford Example 55 (85 mg, 64%) as a white solid. m/z (ESI, +ve ion)=477.10 [M+H]⁺. ¹H NMR (400 MHz, DMSO-d₆) δ 8.78 (s, 1H), 8.67 (s, 1H), 7.93-7.91 (m, 2H), 7.65-7.58 (m, 4H), 7.56-7.48 (m, 1H), 7.44 (s, 1H), 7.22-7.18 (m, 2H), 4.43 (t, J=6.8 Hz, 2H), 3.33-3.17 (m, 2H), 3.04 (s, 3H), 2.51-2.37 (m, 2H).

### Example 58. 1-[3-(4-Fluorophenyl)-4-(6-phenylfuro[2,3-d]pyrimidin-4-yl)-1H-pyrazol-1-yl]propan-2-ol

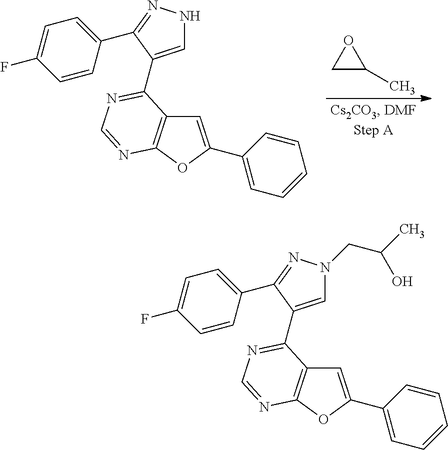

To a stirred solution of 4-[3-(4-Fluorophenyl)-1H-pyrazol-4-yl]-6-phenylfuro[2,3-d]pyrimidine (Intermediate N, 100 mg, 0.281 mmol, 1 equiv) and Cs₂CO₃ (183 mg, 0.562 mmol, 2 equiv) in DMF (1 mL) was added propylene oxide (24.5 mg, 0.422 mmol, 1.5 equiv) at room temperature under nitrogen atmosphere. The resulting mixture was stirred for overnight at 70° C. under nitrogen atmosphere then cooled down to room temperature. The reaction was quenched with water at room temperature. The resulting mixture was extracted with EtOAc (3×20 mL). The combined organic layers were washed with brine (2×25 mL), dried over anhydrous Na₂SO₄. After filtration, the filtrate was concentrated under reduced pressure. The crude product (120 mg) was purified by prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 43% B to 53% B in 8 min, 53% B; Wavelength: 254 nm; RT1 (min): 7 to afford Example 58 (54.7 mg, 47%) as a white solid. m/z (ESI, +ve ion)=415.15 [M+H]⁺. ¹H NMR (400 MHz, Chloroform-d) δ 8.92 (s, 1H), 8.19-8.15 (m, 1H), 7.67-7.65 (m, 2H), 7.57-7.54 (m, 2H), 7.48-7.28 (m, 3H), 7.12-7.07 (m, 2H), 6.15-6.10 (m, 1H), 4.43-4.32 (m, 2H), 4.19-4.14 (m, 1H), 1.37-1.35 (d, J=6.4 Hz, 3H).

### Example 60 (Intermediate N). 4-[3-(4-Fluorophenyl)-1H-pyrazol-4-yl]-6-phenylfuro[2,3-d]pyrimidine

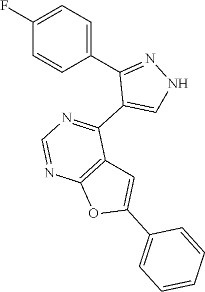 [SMILES: Fc1ccc(-c2n[nH]cc2-c2ncnc3oc(-c4ccccc4)cc23)cc1]

### Example 61. 3-[3-(4-Fluorophenyl)-4-(6-phenylfuro[2,3-d]pyrimidin-4-yl)-1H-pyrazol-1-yl]-N,N-dimethylpropane-1-sulfonamide

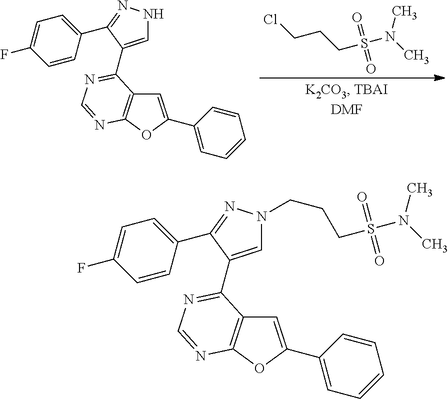

To a stirred solution of 4-[3-(4-Fluorophenyl)-1H-pyrazol-4-yl]-6-phenylfuro[2,3-d]pyrimidine (Intermediate N, 150 mg, 0.421 mmol, 1 equiv) and TBAI (15.55 mg, 0.042 mmol, 0.1 equiv) in DMF (2.5 mL) were added K₂CO₃ (116 mg, 0.84 mmol, 2 equiv) and 3-chloro-N,N-dimethylpropane-1-sulfonamide (94 mg, 0.51 mmol, 1.2 equiv) at room temperature under nitrogen atmosphere. The resulting mixture was stirred for additional 16 h at room temperature then quenched by the addition of water (10 mL) at 0° C. The resulting mixture was extracted with EtOAc (3×50 mL). The combined organic layers were washed with brine (3×30 mL), dried over anhydrous Na₂SO₄. After filtration, the filtrate was concentrated under reduced pressure. The crude product was purified by Prep-HPLC with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 45% B to 60% B in 8 min, 60% B; Wavelength: 254 nm; RT1 (min): 8 to afford Example 61 (160 mg, 75%) as a white solid. m/z (ESI, +ve ion)=506.20 [M+H]⁺.

¹H NMR (400 MHz, DMSO-d₆) δ 8.78 (s, 1H), 8.66 (s, 1H), 7.93-7.91 (m, 2H), 7.64-7.56 (m, 4H), 7.52-7.48 (m, 1H), 7.43 (s, 1H), 7.22-7.18 (m, 2H), 4.42 (t, J=6.8 Hz, 2H), 3.22-3.18 (m, 2H), 2.80 (s, 5H), 2.39-2.32 (m, 2H).

### Example 62. 4-[3-(4-Fluorophenyl)-4-(6-phenylfuro[2,3-d]pyrimidin-4-yl)-1H-pyrazol-1-yl]butan-2-ol

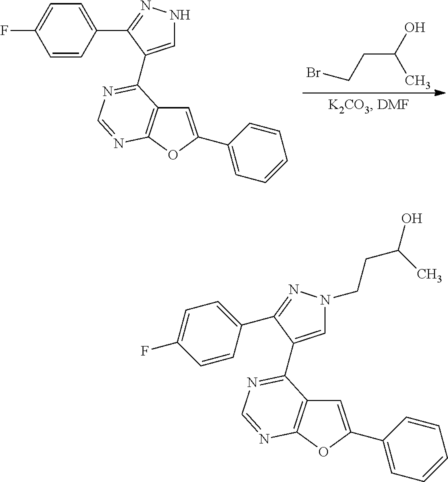

To a stirred solution of 4-[3-(4-Fluorophenyl)-1H-pyrazol-4-yl]-6-phenylfuro[2,3-d]pyrimidine (Intermediate N, 100 mg, 0.28 mmol, 1 equiv) in DMF (1.5 mL) was added K₂CO₃ (78 mg, 0.56 mmol, 2 equiv) at room temperature under nitrogen atmosphere. The resulting mixture was stirred for 10 min at room temperature under nitrogen atmosphere. To the above mixture was added 4-bromobutan-2-ol (51.5 mg, 0.34 mmol, 1.2 equiv) at room temperature. The resulting mixture was stirred for additional 16 h at room temperature then quenched by the addition of water (10 mL). The resulting mixture was extracted with EtOAc (3×50 mL). The combined organic layers were washed with brine (3×30 mL), dried over anhydrous Na₂SO₄. After filtration, the filtrate was concentrated under reduced pressure. The crude product was purified by prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 45% B to 55% B in 8 min, 55% B; Wavelength: 254 nm; RT1 (min): 7.28 to afford Example 62 (81 mg, 67%) as a white solid. m/z (ESI, +ve ion)=429.20 [M+H]⁺. ¹H-NMR (400 MHz, Chloroform-d) δ 8.90 (s, 1H), 8.11 (s, 1H), 7.65 (d, J=8.0 Hz, 2H), 7.56-7.53 (m, 2H), 7.47-7.41 (m, 3H), 7.11-7.07 (m, 2H), 6.12 (s, 1H), 4.52-4.43 (m, 2H), 3.95-3.90 (m, 1H), 2.85 (s, 1H), 2.22-2.17 (m, 2H), 2.02-1.97 (m, 1H), 1.30 (d, J=6.0 Hz, 3H).

### Example 63. 4-[3-(4-Fluorophenyl)-1-methyl-1H-pyrazol-4-yl]-6-phenylfuro[2,3-d]pyrimidine

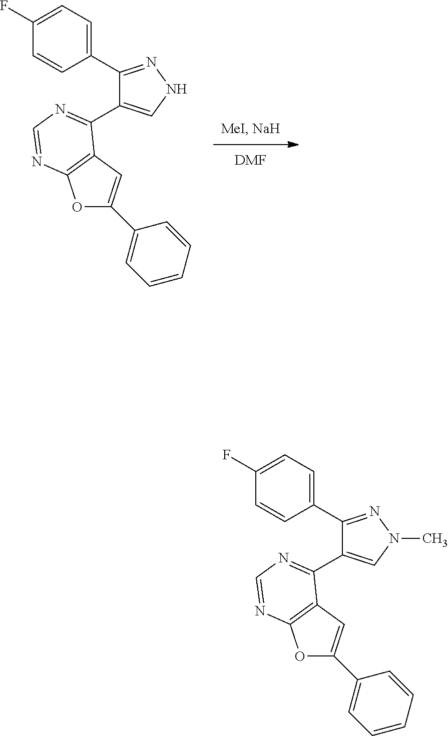

Sodium hydride (16.8 mg, 421 μmol, 1.5 equiv., 60% in dispersion in mineral oil) was added to a solution of 4-[3-(4-fluorophenyl)-1H-pyrazol-4-yl]-6-phenylfuro[2,3-d]pyrimidine (Intermediate N, 100 mg, 281 μmol) in DMF (2.81 mL) at 0° C. The reaction mixture was stirred for 30 min at 0° C., then iodomethane (21.2 μL, 337 μmol, 1.2 equiv.) was added. The mixture was stirred for 16 h at room temperature then diluted with water. The aqueous mixture was extracted with EtOAc (3×20 mL). The combined organic layers were washed with brine, dried (Na₂SO₄), filtered and concentrated under reduced pressure. The residue was purified by column chromatography (EtOAc in hexanes, 0-70%, a gradient elution) to provide the title compound (47.0 mg, 45%) as a solid. ¹H NMR (400 MHz, DMSO-d₆) δ 8.76 (s, 1H), 8.61 (s, 1H), 7.95-7.88 (m, 2H), 7.63-7.53 (m, 4H), 7.52-7.46 (m, 1H), 7.45 (s, 1H), 7.19 (t, J=9.0 Hz, 2H), 4.02 (s, 3H). m/z (ESI, +ve ion)=371.1 [M+H]⁺.

### Example 64. 4-{3-(4-Fluorophenyl)-1-[2-(oxetan-3-yl)ethyl]-1H-pyrazol-4-yl}-6-phenylfuro[2,3-d]pyrimidine

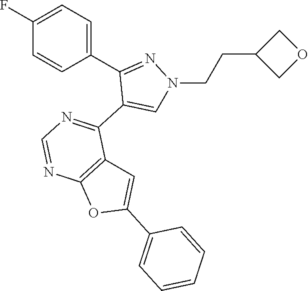 [SMILES: Fc1ccc(-c2nn(CCC3COC3)cc2-c2ncnc3oc(-c4ccccc4)cc23)cc1]

### Step 1. 2-(Oxetan-3-yl)ethyl 4-methylbenzenesulfonate

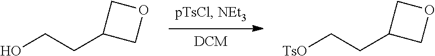

Triethylamine (270 μL, 1.92 mmol, 2.0 equiv.) was added to a degassed mixture of p-toluenesulfonyl chloride (224 mg, 1.15 mmol, 1.2 equiv.) and 2-(oxetan-3-yl)ethanol (100 mg, 960 μmol) in DCM (4.00 mL). The mixture was stirred for 2 d at room temperature then diluted with water (30 mL). The aqueous layer was extracted with DCM (3×30 mL). The combined organic layers were washed with brine then concentrated. The residue was purified by column chromatography (EtOAc in hexanes, 0-100%, a gradient elution) to provide the title compound (171 mg, 70%) as an oil. m/z (ESI, +ve ion)=257.2 [M+H]⁺

### Step 2. 4-(3-(4-Fluorophenyl)-1-(2-(oxetan-3-yl)ethyl)-1H-pyrazol-4-yl)-6-phenylfuro[2,3-d]pyrimidine

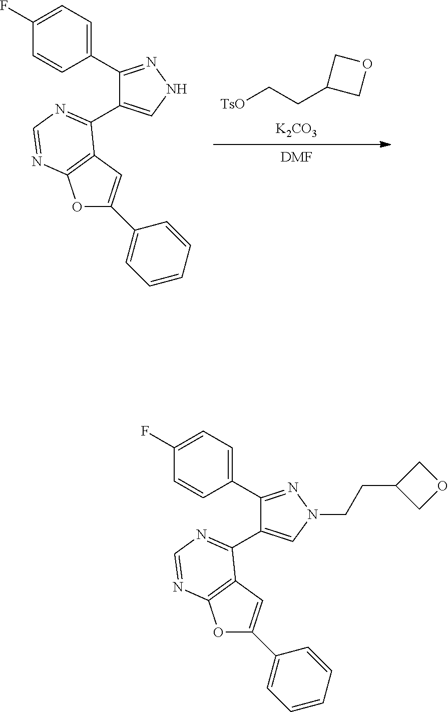

Potassium carbonate (79.1 mg, 561 μmol, 2.0 equiv.) was added to a mixture of 4-[3-(4-fluorophenyl)-1H-pyrazol-4-yl]-6-phenylfuro[2,3-d]pyrimidine (Intermediate N, 100 mg, 281 μmol) and 2-(oxetan-3-yl)ethyl 4-methylbenzenesulfonate (79.1 mg, 309 μmol, 1.1 equiv.) in DMF (2.80 mL). The mixture was stirred for 15 h at room temperature then at 60° C. for 15 h. The mixture was diluted with water (20 mL) and extracted with EtOAc (3×10 mL). The combined organic layers were washed with water, brine, dried (Na₂SO₄), filtered then concentrated. The residue was purified by column chromatography (EtOAc in hexanes, 0-100%, a gradient elution) to provide the title compound (47.0 mg, 38%) as a solid. ¹H NMR (400 MHz, DMSO-d₆) δ 8.76 (s, 1H), 8.64 (s, 1H), 7.92 (d, J=7.4 Hz, 2H), 7.65-7.53 (m, 4H), 7.50 (t, J=7.3 Hz, 1H), 7.39 (s, 1H), 7.19 (t, J=8.8 Hz, 2H), 4.63 (dd, J=7.8, 6.0 Hz, 2H), 4.31-4.16 (m, 4H), 3.10-2.97 (m, 1H), 2.29 (q, J=7.1 Hz, 2H). m/z (ESI, +ve ion)=441.1 [M+H]⁺.

### Example 65. 3-[3-(4-Fluorophenyl)-4-(6-phenylfuro[2,3-d]pyrimidin-4-yl)-1H-pyrazol-1-yl]propane-1-sulfonamide

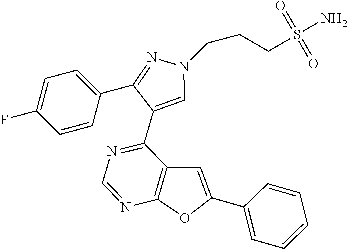 [SMILES: NS(=O)(=O)CCCn1cc(-c2ncnc3oc(-c4ccccc4)cc23)c(-c2ccc(F)cc2)n1]

### Step 1. (E)-N′-(3-Bromopropanesulfonyl)-N,N-dimethylmethanimidamide

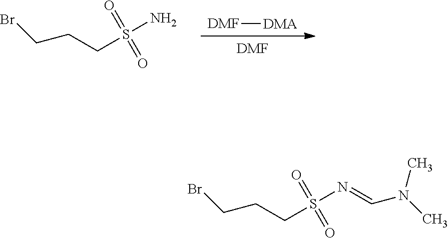

To a stirred solution of 3-bromopropane-1-sulfonamide (100 mg, 0.50 mmol, 1 equiv) in DMF (0.66 mL) was added DMF-DMA (65 mg, 0.54 mmol, 1.1 equiv) dropwise at room temperature under nitrogen atmosphere. The resulting mixture was stirred for 1 h at room temperature under nitrogen atmosphere then extracted with EtOAc (3×50 mL). The combined organic layers were washed with brine (2×20 mL), dried over anhydrous Na₂SO₄. After filtration, the filtrate was concentrated under reduced pressure. The residue was purified by silica gel column chromatography, eluted with PE:EA (5:1) to afford the title compound (123 mg, 97%) as a yellow oil. m/z (ESI, +ve ion)=257.05, 259.05 [M+H]⁺.

### Step 2. (E)-N′-{3-[3-(4-Fluorophenyl)-4-{6-phenylfuro[2,3-d]pyrimidin-4-yl}pyrazol-1-yl]propanesulfonyl}-N,N-dimethylmethanimidamide

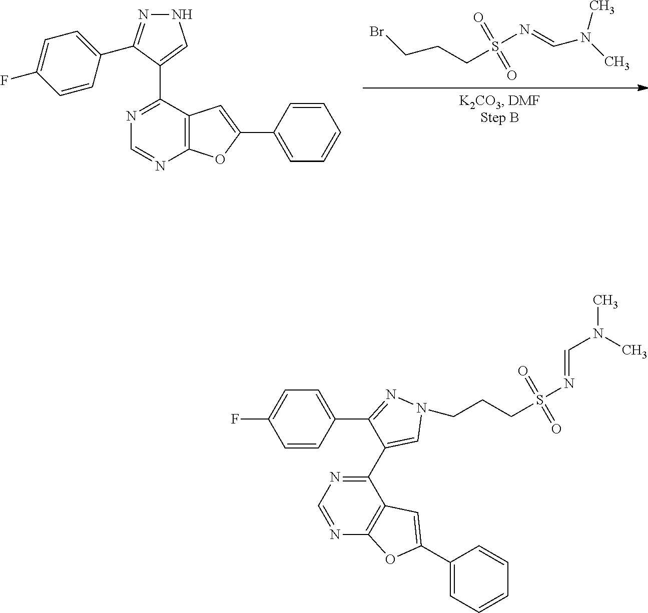

To a stirred solution of 4-[3-(4-Fluorophenyl)-1H-pyrazol-4-yl]-6-phenylfuro[2,3-d]pyrimidine (Intermediate N, 120 mg, 0.34 mmol, 1.0 equiv) in DMF (4 mL) were added K₂CO₃ (93 mg, 0.67 mmol, 2 equiv) at room temperature under nitrogen atmosphere. The resulting mixture was stirred for 10 min at room temperature under nitrogen atmosphere. To the above mixture was added (E)-N′-(3-bromopropanesulfonyl)-N,N-dimethylmethanimidamide (129.89 mg, 0.506 mmol, 1.5 equiv) at room temperature. The resulting mixture was stirred for additional 16 h at room temperature then quenched by the addition of water (20 mL) at 0° C. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE:EtOAc (5:1) to afford the title compound (150 mg, 84%) as a yellow solid. m/z (ESI+ve ion)=533.25 [M+H]⁺.

### Step 3. 3-[3-(4-Fluorophenyl)-4-(6-phenylfuro[2,3-d]pyrimidin-4-yl)-1H-pyrazol-1-yl]propane-1-sulfonamide

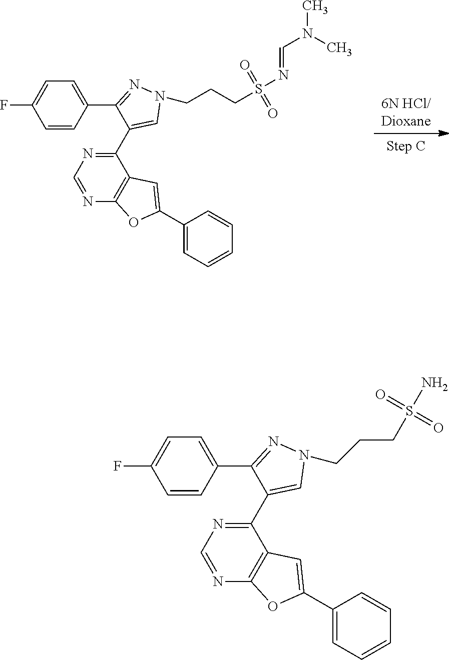 [SMILES: CN(C)/C=N/S(=O)(=O)CCCn1cc(-c2ncnc3oc(-c4ccccc4)cc23)c(-c2ccc(F)cc2)n1 ; "6N HCl/Dioxane" ; "Step C" ; NS(=O)(=O)CCCn1cc(-c2ncnc3oc(-c4ccccc4)cc23)c(-c2ccc(F)cc2)n1]

To a stirred solution of (E)-N′-{3-[3-(4-fluorophenyl)-4-{6-phenylfuro[2,3-d]pyrimidin-4-yl}pyrazol-1-yl]propanesulfonyl}-N,N-dimethylmethanimidamide (95 mg, 0.178 mmol, 1 equiv) in 1,4-dioxane (0.84 mL) was added conc. HCl (0.84 mL) dropwise at room temperature under nitrogen atmosphere. The resulting mixture was stirred for 1 h at 100° C. under nitrogen atmosphere then quenched by the addition of sat. NaHCO₃ (aq., 10 mL) at 0° C. The resulting mixture was extracted with EtOAc (3×100 mL). The combined organic layers were washed with brine (3×100 mL), dried over anhydrous Na₂SO₄. After filtration, the filtrate was concentrated under reduced pressure. The crude product was purified by prep-HPLC with following conditions: Column: XSelect CSH Prep C18 OBD Column, 19×250 mm, 5 m; Mobile Phase A: water (10 mmol/L NH₄HCO₃), Mobile Phase B: ACN; Flow rate: 25 mL/min; Gradient: 45% B to 45% B in 14 min, 45% B; Wavelength: 254 nm; RT1 (min): 13.2 to afford Example 65 (51 mg, 60%) as a white solid. m/z (ESI+ve ion)=478.25 [M+H]⁺. ¹H NMR (400 MHz, DMSO-d₆) δ 8.78 (s, 1H), 8.66 (s, 1H), 7.95-7.88 (m, 2H), 7.67-7.53 (m, 4H), 7.53-7.38 (m, 2H), 7.25-7.15 (m, 2H), 6.91 (s, 2H), 4.43 (t, J=6.8 Hz, 2H), 3.14-3.06 (m, 2H), 2.43-2.31 (m, 2H).

### Example 69. 4-{3-(4-Fluorophenyl)-1-[(oxetan-2-yl)methyl]-1H-pyrazol-4-yl}-6-phenylfuro[2,3-d]pyrimidine

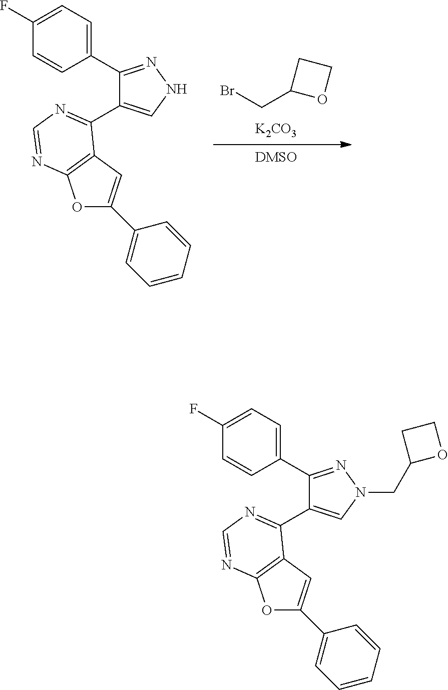

Potassium carbonate (98.9 mg, 702 μmol, 2.5 equiv.) was added to a mixture of 4-[3-(4-fluorophenyl)-1H-pyrazol-4-yl]-6-phenylfuro[2,3-d]pyrimidine (Intermediate N, 100 mg, 281 μmol) in DMSO (1.12 mL) at room temperature. After 5 minutes, 2-(bromomethyl)oxetane (48.1 mg, 309 μmol, 1.1 equiv.) was added, and the reaction mixture was stirred at 25° C. for 16 hours. Additional 2-(bromomethyl)oxetane (26.2 mg, 168 μmol, 0.6 equiv.) and cesium carbonate (148 mg, 449 μmol, 1.6 equiv.) were added and the reaction mixture was heated to 40° C. for 26 hours. The mixture was cooled to room temperature and diluted with water (10 mL) and EtOAc (2 mL). The layers were separated, and the aqueous layer was extracted with additional EtOAc (2×1 mL). The combined organic layers were washed with water (10 mL), dried over Na₂SO₄, filtered, and concentrated under reduced pressure. The residue was purified by column chromatography (EtOAc in hexanes, 0-80%, a gradient elution), to provide the title compound (36.0 mg, 30%) as a solid. ¹H NMR (400 MHz, DMSO-d₆) δ 8.78 (s, 1H), 8.63 (s, 1H), 7.93-7.87 (m, 2H), 7.64-7.58 (m, 2H), 7.56 (dd, J=7.3, 5.9 Hz, 2H), 7.52-7.46 (m, 1H), 7.35 (s, 1H), 7.24-7.16 (m, 2H), 5.22-5.12 (m, 1H), 4.61-4.48 (m, 3H), 4.44-4.35 (m, 1H), 2.80-2.69 (m, 1H), 2.61-2.52 (m, 1H). m/z (ESI, +ve ion)=427.4 [M+H]⁺.

### Example 70. 3-[3-(4-Fluorophenyl)-4-(6-phenylfuro[2,3-d]pyrimidin-4-yl)-1H-pyrazol-1-yl]propanenitrile

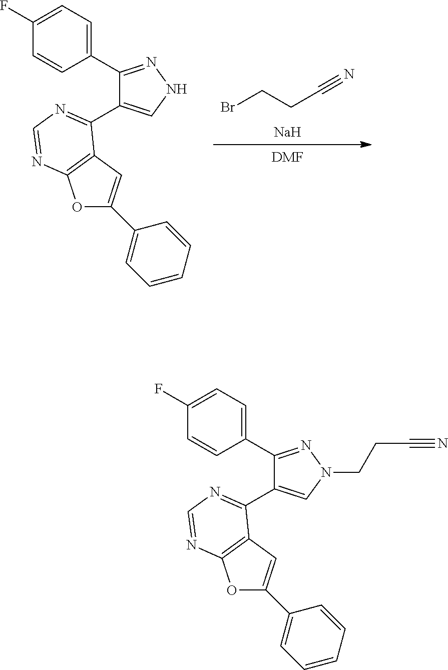

Sodium hydride (16.8 mg, 421 μmol, 1.5 equiv., 60% in dispersion in mineral oil) was added to a solution of 4-[3-(4-fluorophenyl)-1H-pyrazol-4-yl]-6-phenylfuro[2,3-d]pyrimidine (Intermediate N, 100 mg, 281 μmol) in DMF (2.81 mL) at 0° C. The mixture was stirred for 30 min at 0° C. then 3-bromopropionitrile (33.8 μL, 407 μmol, 1.4 equiv.) was added. The mixture was stirred for 16 h at room temperature. Additional sodium hydride (16.8 mg, 421 μmol, 1.5 equiv., 60% in dispersion in mineral oil) was added and the reaction mixture was further stirred at room temperature for 20 h. The mixture was diluted with water (30 mL) and extracted with DCM (3×35 mL). The combined organic layers were washed with brine, dried (Na₂SO₄), filtered and concentrated. The residue was purified by column chromatography (MeOH in DCM, 0-20%, a gradient elution) to provide the title compound (41.2 mg, 36%) as a solid. ¹H NMR (400 MHz, DMSO-d₆) δ 8.80 (s, 1H), 8.73 (s, 1H), 7.92-7.87 (m, 2H), 7.65-7.60 (m, 2H), 7.59-7.54 (m, 2H), 7.53-7.47 (m, 1H), 7.37 (s, 1H), 7.24-7.17 (m, 2H), 4.58 (t, J=6.4 Hz, 2H), 3.25 (t, J=6.4 Hz, 2H). m/z (ESI, +ve ion)=410.4 [M+H]⁺.

### Example 72. (3R)-3-[3-(4-Fluorophenyl)-4-(6-phenylfuro[2,3-d]pyrimidin-4-yl)-1H-pyrazol-1-yl]-1λ6-thiolane-1,1-dione

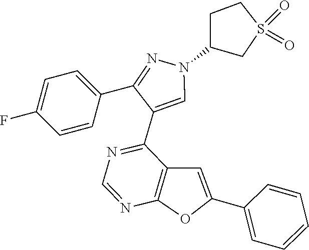

### Example 73. (3S)—3-[3-(4-Fluorophenyl)-4-(6-phenylfuro[2,3-d]pyrimidin-4-yl)-1H-pyrazol-1-yl]-1λ6-thiolane-1,1-dione

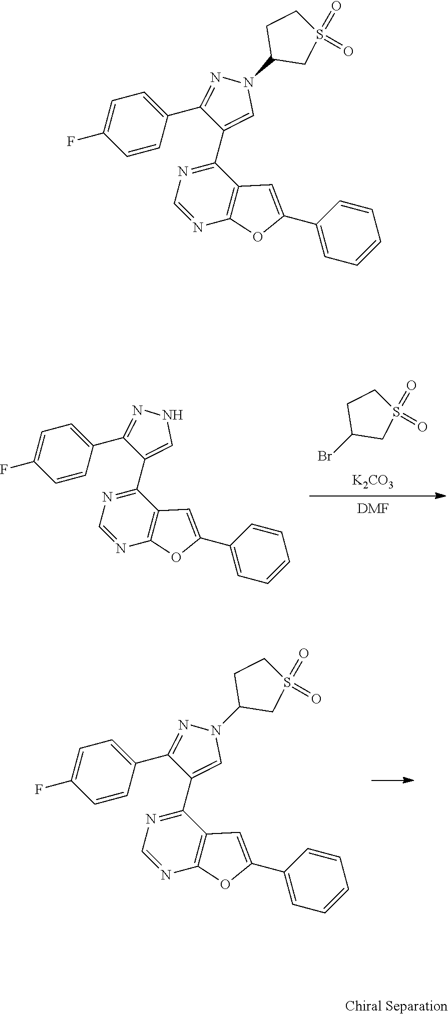

To a stirred solution of 4-[3-(4-fluorophenyl)-1H-pyrazol-4-yl]-6-phenylfuro[2,3-d]pyrimidine (Intermediate N, 100 mg, 0.28 mmol, 1 equiv) and K₂CO₃ (78 mg, 0.56 mmol, 2 equiv) in DMF (4 mL) was added 3-bromo-1,6-thiolane-1,1l-dione (67.03 mg, 0.34 mmol, 1.2 equiv) at room temperature under nitrogen atmosphere. The resulting mixture was stirred for overnight at room temperature under nitrogen atmosphere. The reaction was quenched by the addition of water (10 mL) at room temperature. The resulting mixture was extracted with EtOAc (3×15 mL). The combined organic layers were washed with brine (10 mL), dried over anhydrous Na₂SO₄. After filtration, the filtrate was concentrated under reduced pressure. The crude product (120 mg) was purified by prep-HPLC with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 45% B to 65% B in 8 min, 65% B; Wavelength: 254 nm; RT1 (min): 6.6 to afford Example 41 (46.7 mg, 35%) as a white solid. The mixture (220 mg) was separated by chiral-HPLC with the following conditions: Column: CHIRAL ART Cellulose-SC, 2×25 cm, 5 μm; Mobile Phase A: MTBE (0.1% FA)-HPLC, Mobile Phase B: MeOH:DCM=1:1-HPLC; Flow rate: 20 mL/min; Gradient: 5% B to 5% B in 15.5 min; Wavelength: 220/254 nm; RT1 (min): 10.71; RT2 (min): 14.59; Sample Solvent: MeOH:DCM=1:1-HPLC; Injection Volume: 1.1 mL; Number Of Runs: 6 to afford Example 72 (first peak, 57.1 mg, 21%) (absolute stereochemistry arbitrarily assigned) and Example 73 (second peak, 55.6 mg, 21%) (absolute stereochemistry arbitrarily assigned) as the white solid.

Example 72. m/z (ESI, +ve ion)=475.15 [M+H]⁺. ¹H NMR (400 MHz, Chloroform-d) δ 8.95 (s, 1H), 8.25 (s, 1H), 7.66-7.63 (m, 2H), 7.57-7.53 (m, 2H), 7.49-7.41 (m, 3H), 7.14-7.09 (m, 2H), 6.11 (s, 1H), 5.27-5.23 (m, 1H), 3.84-3.79 (m, 1H), 3.74-3.60 (m, 2H), 3.34-3.27 (m, 1H), 2.96-2.85 (m, 2H).

Example 73. m/z (ESI, +ve ion)=475.15 [M+H]⁺. ¹H NMR (400 MHz, Chloroform-d) δ 8.95 (s, 1H), 8.22 (s, 1H), 7.67-7.64 (m, 2H), 7.57-7.54 (m, 2H), 7.48-7.41 (m, 3H), 7.13-7.09 (m, 2H), 6.12 (s, 1H), 5.27-5.21 (m, 1H), 3.84-3.79 (m, 1H), 3.74-3.60 (m, 2H), 3.34-3.27 (m, 1H), 2.96-2.85 (m, 2H).

### Example 76. 4-[3-(4-Fluorophenyl)-1-(oxan-4-yl)-1H-pyrazol-4-yl]-6-phenylfuro[2,3-d]pyrimidine

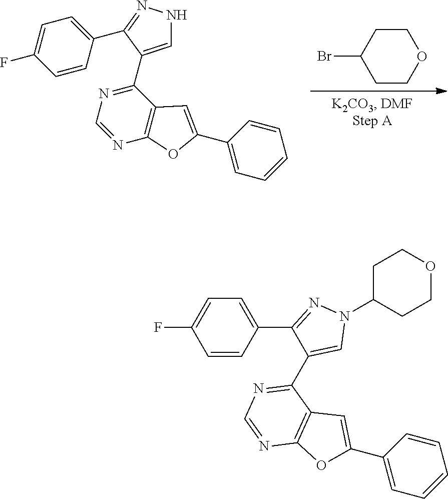

To a stirred solution of 4-[3-(4-fluorophenyl)-1H-pyrazol-4-yl]-6-phenylfuro[2,3-d]pyrimidine (Intermediate N, 100 mg, 0.28 mmol, 1 equiv) in DMF (2.7 mL) were added K₂CO₃ (78 mg, 0.56 mmol, 2 equiv) at room temperature under nitrogen atmosphere. The resulting mixture was stirred for 10 min at room temperature under nitrogen atmosphere. To the above mixture was added 4-bromooxane (111 mg, 0.674 mmol, 2.4 equiv) at room temperature. The resulting mixture was stirred for additional 16 h at 100° C. The reaction was quenched by the addition of water (10 mL) at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The crude product was purified by prep-HPLC with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 50% B to 65% B in 8 min, 65% B; Wavelength: 254 nm; RT1 (min): 6.3 to afford Example 76 (24.2 mg, 19%) as a white solid. m/z (ESI, +ve ion)=441.25 [M+H]⁺. ¹H NMR (400 MHz, Chloroform-d) δ 8.93 (s, 1H), 8.23 (s, 1H), 7.65-7.63 (m, 2H), 7.57-7.54 (m, 2H), 7.47-7.41 (m, 3H), 7.12-7.01 (m, 2H), 6.10 (s, 1H), 4.49-4.45 (m, 1H), 4.28-4.24 (m, 1H), 3.96-3.90 (m, 2H), 3.69-3.63 (m, 1H), 2.36-2.29 (m, 2H), 1.91-1.82 (m, 2H).

### Example 77. 2-[3-(4-Fluorophenyl)-4-(6-phenylfuro[2,3-d]pyrimidin-4-yl)-1H-pyrazol-1-yl]-2-methylpropanamide

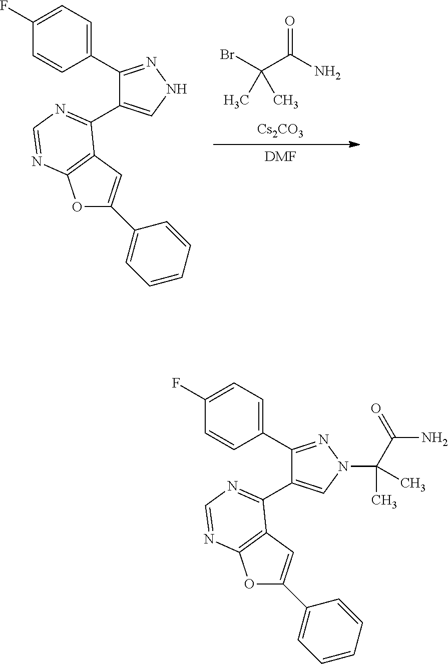

2-Bromo-2-methylpropionamide (57.0 mg, 337 μmol, 1.2 equiv.) was added to a mixture of 4-[3-(4-fluorophenyl)-1H-pyrazol-4-yl]-6-phenylfuro[2,3-d]pyrimidine (Intermediate N, 100 mg, 281 μmol) and cesium carbonate (231 mg, 702 μmol, 2.5 equiv.) in DMF (2.0 mL) at room temperature. The mixture was stirred for 16 h at room temperature. The mixture was diluted with water (10 mL), brine (10 mL) and extracted with EtOAc (3×40 mL). The combined organic layers were washed with brine (10 mL), dried over Na₂SO₄, filtered and concentrated. The residue was purified by column chromatography (MeOH in DCM, 0-4%, a gradient elution) followed by reverse phase chromatography (C18 column, MeCN in water with 10 mM ammonium formate, 20-70%, a gradient elution) to provide the title compound (25.1 mg, 20%) as a solid. ¹H NMR (400 MHz, DMSO-d₆) δ 8.79 (s, 1H), 8.55 (s, 1H), 7.92 (app d, J=7.4 Hz, 2H), 7.61-7.53 (m, 4H), 7.52-7.46 (m, 1H), 7.36 (s, 1H), 7.33 (br s, 1H), 7.23-7.15 (m, 3H), 1.87 (s, 6H). m/z (ESI, +ve ion)=442.4 [M+H]⁺.

### Example 78. 2,2-Difluoro-3-[3-(4-fluorophenyl)-4-(6-phenylfuro[2,3-d]pyrimidin-4-yl)-1H-pyrazol-1-yl]propan-1-ol

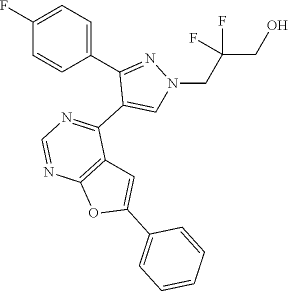 [SMILES: OCC(F)(F)Cn1cc(-c2ncnc3oc(-c4ccccc4)cc23)c(-c2ccc(F)cc2)n1]

### Step A. 4-(1-(3-((tert-Butyldiphenylsilyl)oxy)-2,2-difluoropropyl)-3-(4-fluorophenyl)-1H-pyrazol-4-yl)-6-phenylfuro[2,3-d]pyrimidine

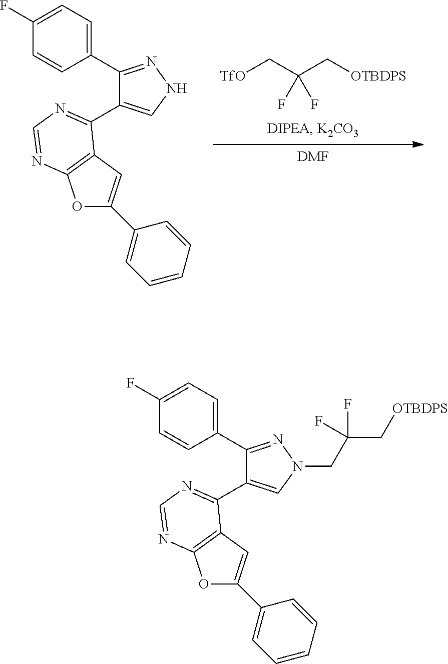

N,N-Diisopropylethylamine (148 μL, 842 μmol, 3.0 equiv.) was added to a mixture of 4-[3-(4-fluorophenyl)-1H-pyrazol-4-yl]-6-phenylfuro[2,3-d]pyrimidine (Intermediate N, 100 mg, 281 μmol) and 3-((tert-butyldiphenylsilyl)oxy)-2,2-difluoropropyl trifluoromethanesulfonate (166 μL, 421 μmol, 1.5 equiv.) in DMF (2.81 mL). The mixture was stirred for 16 h at 80° C. Potassium carbonate (79.1 mg, 561 μmol, 2.0 equiv.) was added and the mixture was stirred for 20 h at 70° C. Additional potassium carbonate (79.1 mg, 561 μmol, 2.0 equiv.) and 3-((tert-butyldiphenylsilyl)oxy)-2,2-difluoropropyl trifluoromethanesulfonate (166 μL, 421 μmol, 1.5 equiv.) were added and the reaction mixture was stirred for 20 h at 70° C. and cooled to room temperature. The mixture was diluted with water (20 mL) and extracted with EtOAc (3×10 mL). The combined organic layers were washed with water, brine, dried over Na₂SO₄, filtered and concentrated. The residue was purified by column chromatography twice (EtOAc in hexanes, 0-20%, a gradient elution) to provide the title compound (100 mg, 41%) as an oil. m/z (ESI, +ve ion)=689.9 [M+H]⁺.

### Step 2. 2,2-Difluoro-3-[3-(4-fluorophenyl)-4-(6-phenylfuro[2,3-d]pyrimidin-4-yl)-1H-pyrazol-1-yl]propan-1-ol

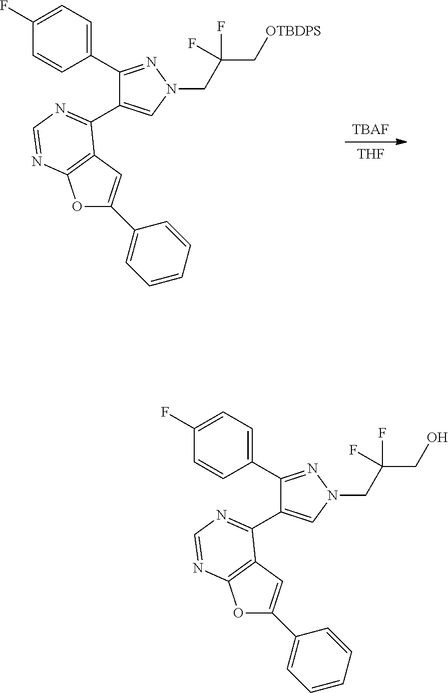

Tetrabutylammonium fluoride (174 μL, 174 μmol, 1.2 equiv., 1.0 M in THF) was added to a mixture of 4-(1-(3-((tert-butyldiphenylsilyl)oxy)-2,2-difluoropropyl)-3-(4-fluorophenyl)-1H-pyrazol-4-yl)-6-phenylfuro[2,3-d]pyrimidine (100 mg, 145 μmol). The reaction mixture was stirred for 16 h at room temperature then diluted with a saturated aqueous solution of NH₄Cl (20 mL) and extracted with EtOAc (3×10 mL). The combined organic layers were washed with saturated aqueous solution of NH₄C₁, brine, dried over Na₂SO₄, filtered, and concentrated under reduced pressure. The residue was purified by column chromatography (EtOAc in hexanes, 0-100%, a gradient elution) to provide the title compound (36.0 mg, 55%) as a solid. ¹H NMR (400 MHz, DMSO-d₆) δ 8.80 (s, 1H), 8.66 (s, 1H), 7.89 (d, J=7.3 Hz, 2H), 7.60 (dd, J=8.7, 5.7 Hz, 2H), 7.56 (t, J=7.5 Hz, 2H), 7.49 (t, J=7.3 Hz, 1H), 7.30 (s, 1H), 7.21 (t, J=8.9 Hz, 2H), 5.82 (t, J=6.1 Hz, 1H), 4.86 (t, J=14.2 Hz, 2H), 3.77 (td, J=13.6, 6.0 Hz, 2H). m/z (ESI, +ve ion)=451.4 [M+H]⁺.

### Example 90. (R)-4-{3-(4-Fluorophenyl)-1-[(3R)-oxolan-3-yl]-1H-pyrazol-4-yl}-6-phenylfuro[2,3-d]pyrimidine

### Example 91. (S)-4-{3-(4-Fluorophenyl)-1-[(3S)-oxolan-3-yl]-1H-pyrazol-4-yl}-6-phenylfuro[2,3-d]pyrimidine

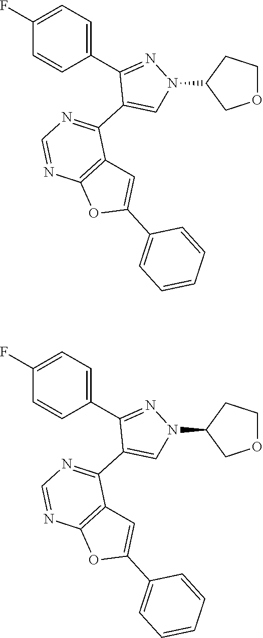

### Step A. 4-[3-(4-Fluorophenyl)-1-(oxolan-3-yl)-1H-pyrazol-4-yl]-6-phenylfuro[2,3-d]pyrimidine

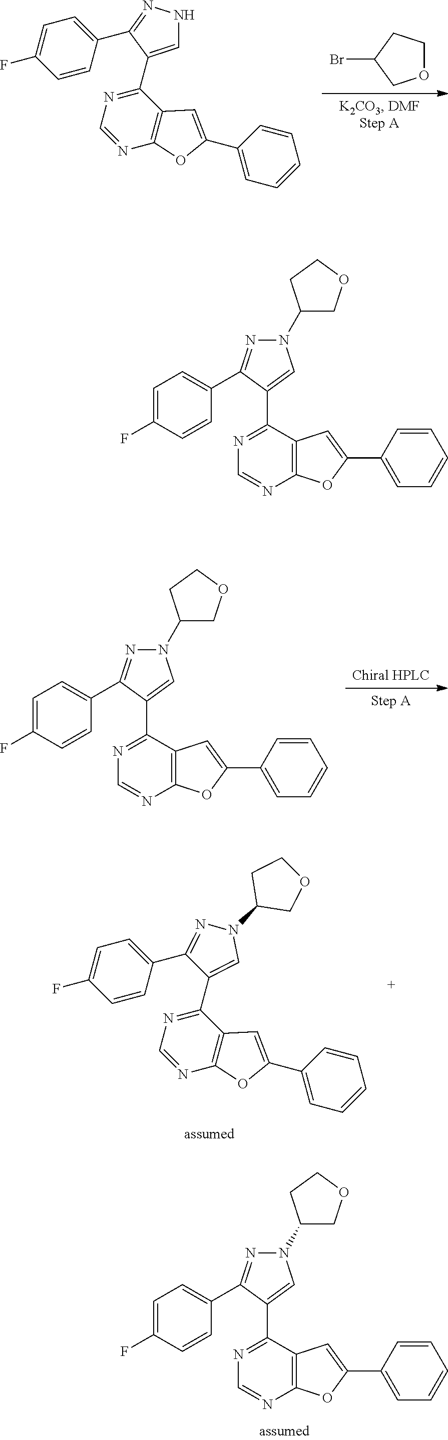

To a stirred solution of 4-[3-(4-fluorophenyl)-1H-pyrazol-4-yl]-6-phenylfuro[2,3-d]pyrimidine (Intermediate N, 200 mg, 0.561 mmol, 1 equiv) in DMF (5.5 mL) was added K₂CO₃ (155.13 mg, 1.122 mmol, 2 equiv) at room temperature under nitrogen atmosphere. The resulting mixture was stirred for 10 min at room temperature under nitrogen atmosphere. To the above mixture was added 3-bromooxolane (101.70 mg, 0.673 mmol, 1.2 equiv) dropwise at room temperature. The resulting mixture was stirred for additional 16 h at room temperature then quenched by the addition of water (10 mL) at 0° C. The resulting mixture was extracted with EtOAc (3×50 mL). The combined organic layers were washed with brine (3×50 mL), dried over anhydrous Na₂SO₄. After filtration, the filtrate was concentrated under reduced pressure. The crude product was purified by prep-HPLC with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 50% B to 65% B in 8 min, 65% B; Wavelength: 254 nm; RT1 (min): 6.7 to afford Example 85 (110 mg, 46%) as a white solid.

The racemic was separated by Chiral-HPLC with the following conditions: Column: CHIRALPAK IC-3, 4.6×50 mm, 3 um; Mobile Phase A: Hex (0.1% DEA): (EtOH:DCM=1:1)=85:15; Flow rate: 1 mL/min; Gradient: 0% B to 0% B; Injection Volume: 5 ul mL to afford to afford Example 90 (first peak, 46.6 mg, 19%) (absolute stereochemistry arbitrarily assigned) and Example 91 (second peak, 44.5 mg, 18%) (absolute stereochemistry arbitrarily assigned) as a white solid.

Example 90. m/z (ESI, +ve ion)=427.15 [M+H]⁺. ¹H NMR (400 MHz, DMSO-d₆) δ 8.79 (s, 1H), 8.62 (s, 1H), 7.93-7.91 (m, 2H), 7.62-7.48 (m, 5H), 7.37 (s, 1H), 7.22-7.17 (m, 2H), 5.24-5.18 (m, 1H), 4.14-4.07 (m, 3H), 3.93-3.88 (m, 1H), 2.53-2.48 (m, 2H).

Example 91. m/z (ESI, +ve ion)=427.15 [M+H]⁺. ¹H NMR (400 MHz, DMSO-d₆) δ 8.79 (s, 1H), 8.62 (s, 1H), 7.93-7.91 (m, 2H), 7.63-7.50 (m, 5H), 7.37 (s, 1H), 7.22-7.17 (m, 2H), 5.23-5.18 (m, 1H), 4.12-4.07 (m, 3H), 3.93-3.90 (m, 1H), 2.53-2.48 (m, 2H).

### Example 94. 2-[3-(4-Fluorophenyl)-4-(6-phenylfuro[2,3-d]pyrimidin-4-yl)-1H-pyrazol-1-yl]ethan-1-ol

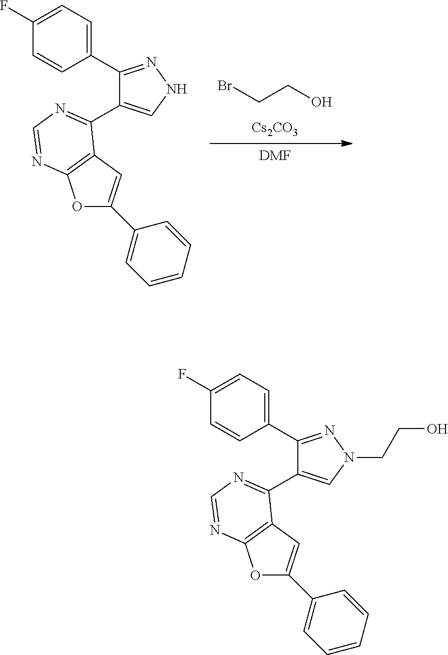

Sodium hydride (16.8 mg, 421 μmol, 1.5 equiv., 60% in dispersion in mineral oil) was added to a mixture of 4-[3-(4-fluorophenyl)-1H-pyrazol-4-yl]-6-phenylfuro[2,3-d]pyrimidine (Intermediate N, 100 mg, 281 μmol) in DMF (2.00 mL) at 0° C. The mixture was stirred for 30 min at 0° C. then 2-bromoethanol (29.4 μL, 415 μmol, 1.5 equiv.) was added. The mixture was stirred for 16 h at room temperature then at 50° C. for 20 h. Additional 2-bromoethanol (19.9 μL, 281 μmol, 1.0 equiv.) was added and the mixture stirred at 50° C. for 20 h. The mixture was diluted with water (10 mL) and extracted with EtOAc (3×15 mL). The combined organic layers were washed with water (10 mL), brine (10 mL), then dried over Na₂SO₄, filtered and concentrated. The residue was purified by column chromatography (MeOH in DCM, 04%, a gradient elution). The material was further purified by column chromatography (EtOAc in hexanes, 0-100%, a gradient elution) to provide the title compound (72.4 mg, 64%) as a solid. ¹H NMR (400 MHz, DMSO-d₆) δ 8.77 (s, 1H), 8.60 (s, 1H), 7.94-7.87 (m, 2H), 7.63-7.54 (m, 4H), 7.50 (dd, J=10.4, 4.2 Hz, 1H), 7.38 (s, 1H), 7.19 (t, J=8.9 Hz, 2H), 5.06 (t, J=5.3 Hz, 1H), 4.32 (t, J=5.6 Hz, 2H), 3.89 (q, J=5.5 Hz, 2H). m/z (ESI, +ve ion)=401.3 [M+H]⁺.

### Example 95. 1-[3-(3-Fluorophenyl)-4-(6-phenylfuro[2,3-d]pyrimidin-4-yl)-1H-pyrazol-1-yl]-2-methylpropan-2-ol

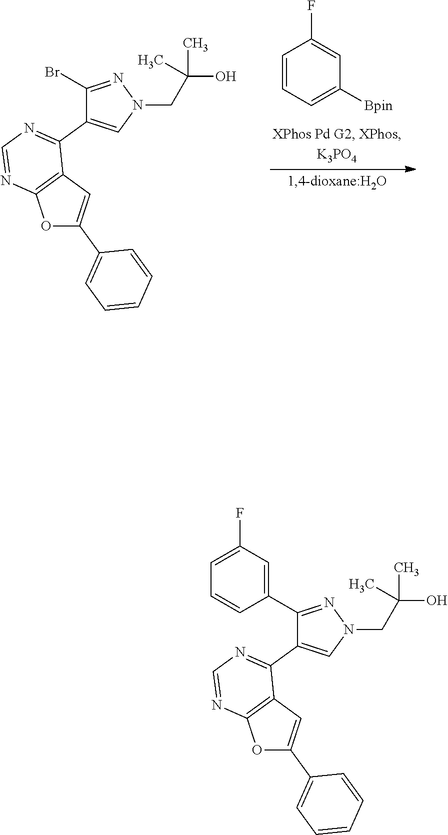

1-(3-Bromo-4-(6-phenylfuro[2,3-d]pyrimidin-4-yl)-1H-pyrazol-1-yl)-2-methylpropan-2-ol (Intermediate P, 100 mg, 242 μmol), 3-fluorophenylboronic acid (67.7 mg, 474 μmol, 2.0 equiv.), XPhos Pd G2 (13.3 mg, 16.9 μmol, 0.07 equiv.), XPhos (13.1 mg, 26.6 μmol, 0.11 equiv.) and potassium phosphate tribasic (105 mg, 484 μmol, 2.0 equiv.) were purged with nitrogen and evacuated three times, then 1,4-dioxane (807 μL) and H₂O (161 μL) were then added. The mixture was purged with nitrogen and evacuated three times then heated at 100° C. for 42 h. The mixture was cool to room temperature, diluted with EtOAc and then filtered. The filtrate was washed with brine, dried over Na₂SO₄, filtered, and concentrated under reduced pressure. The residue was purified by reverse phase chromatography (C18 column, MeCN in water with 10 mM ammonium formate, 5-70%, a gradient elution) to provide the title compound (32.0 mg, 31%) as a solid. ¹H NMR (400 MHz, DMSO-d₆) δ 8.80 (s, 1H), 8.53 (s, 1H), 7.92-7.89 (m, 2H), 7.59-7.54 (m, 2H), 7.51-7.47 (m, 1H), 7.43-7.38 (m, 3H), 7.35 (s, 1H), 7.22-7.16 (m, 1H), 4.87 (s, 1H), 4.21 (s, 2H), 1.20 (s, 6H). m/z (ESI, +ve ion)=429.3 [M+H]⁺.

### Example 96. (R)-3-{[3-(4-Fluorophenyl)-4-(6-phenylfuro[2,3-d]pyrimidin-4-yl)-1H-pyrazol-1-yl]methyl}-16-thiolane-1,1-dione

### Example 97. (S)-3-{[3-(4-Fluorophenyl)-4-(6-phenylfuro[2,3-d]pyrimidin-4-yl)-1H-pyrazol-1-yl]methyl}-16-thiolane-1,1-dione

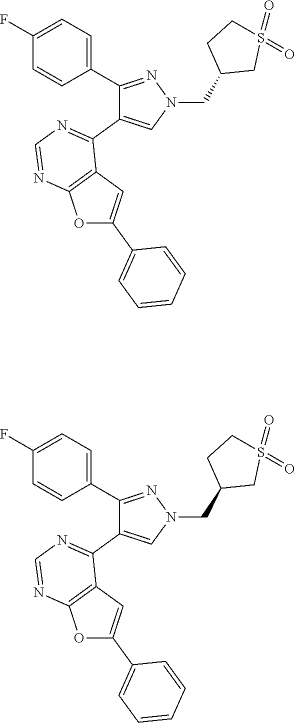 [SMILES: O=S1(=O)CC[C@@H](Cn2cc(-c3ncnc4oc(-c5ccccc5)cc34)c(-c3ccc(F)cc3)n2)C1 ; O=S1(=O)CC[C@H](Cn2cc(-c3ncnc4oc(-c5ccccc5)cc34)c(-c3ccc(F)cc3)n2)C1]

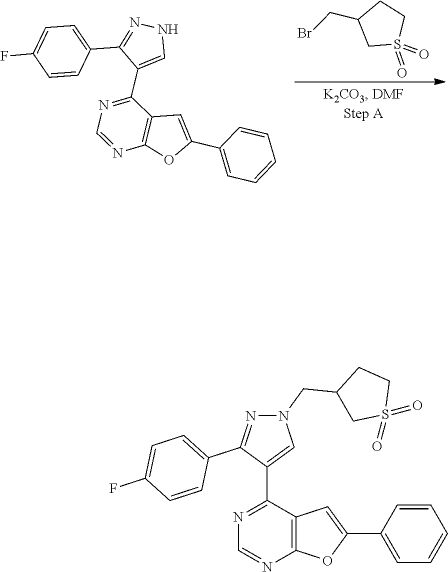 [SMILES: Fc1ccc(-c2n[nH]cc2-c2ncnc3oc(-c4ccccc4)cc23)cc1 ; O=S1(=O)CCC(CBr)C1 ; O=S1(=O)CCC(Cn2cc(-c3ncnc4oc(-c5ccccc5)cc34)c(-c3ccc(F)cc3)n2)C1]

To a stirred solution of 4-[3-(4-fluorophenyl)-1H-pyrazol-4-yl]-6-phenylfuro[2,3-d]pyrimidine (Intermediate N, 100 mg, 0.281 mmol, 1 equiv) and K₂CO₃ (77.56 mg, 0.562 mmol, 2 equiv) in DMF (2 mL) was added 3-(bromomethyl)-1,6-thiolane-1,1-dione (71.76 mg, 0.337 mmol, 1.2 equiv) in portions at room temperature under nitrogen atmosphere. The resulting mixture was stirred for overnight at room temperature under nitrogen atmosphere then quenched by the addition of water (10 mL) at room temperature. The resulting mixture was extracted with EtOAc (3×15 mL). The combined organic layers were washed with brine (10 mL), dried over anhydrous Na₂SO₄. After filtration, the filtrate was concentrated under reduced pressure. The crude product (120 mg) was purified by prep-HPLC with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 45% B to 60% B in 8 min, 60% B; Wavelength: 254 nm; RT1 (min): 6.4 to afford Example 53 (33.6 mg, 25%) as a white solid. The racemic (260 mg) was purified by Chiral-HPLC with the following conditions: Column: CHIRALPAK IE, 2×25 cm, 5 μm; Mobile Phase A: Hex (0.5% 2M NH₃-MeOH)-HPLC, Mobile Phase B: MeOH:DCM=1:1-HPLC; Flow rate: 20 mL/min; Gradient: 30% B to 30% B in 30 min; Wavelength: 220/254 nm; RT1 (min): 24.20; RT2 (min): 27.62; Sample Solvent: MeOH:DCM=1:1 to afford Example 96 (first peak, 42 mg, 15%) (absolute stereochemistry arbitrarily assigned) as the white solid and Example 97 (second peak, 48.2 mg, 18%) (absolute stereochemistry arbitrarily assigned) as the white solid.

Example 96. m/z (ESI, +ve ion)=489.20 [M+H]⁺. ¹H NMR (400 MHz, Chloroform-d) δ 8.94 (s, 1H), 8.14 (s, 1H), 7.66-7.64 (m, 2H), 7.56-7.53 (m, 2H), 7.48-7.41 (m, 3H), 7.13-7.09 (m, 2H), 6.09 (s, 1H), 4.42-4.40 (d, J=6.8 Hz, 2H), 3.38-3.14 (m, 3H), 3.16-3.11 (m, 1H), 3.06-3.01 (m, 1H), 2.47-2.46 (d, J=4.6, 1H), 2.18-2.11 (m, 1H).

Example 97. m/z (ESI, +ve ion)=489.20 [M+H]⁺. ¹H NMR (400 MHz, Chloroform-d) δ 8.94 (s, 1H), 8.14 (s, 1H), 7.66-7.64 (m, 2H), 7.60-7.52 (m, 2H), 7.48-7.29 (m, 3H), 7.13-7.09 (m, 2H), 6.09 (s, 1H), 4.41-4.40 (d, J=7.0 Hz, 2H), 3.38-3.14 (m, 4H), 3.14-3.01 (m, 1H), 2.47-2.45 (m, 1H), 2.15-2.10 (m, 1H).

### Example 101. (R)-4-{3-(4-Fluorophenyl)-1-[(3R)-1-(methanesulfonyl)pyrrolidin-3-yl]-1H-pyrazol-4-yl}-6-phenylfuro[2,3-d]pyrimidine

### Example 102. (S)-4-{3-(4-Fluorophenyl)-1-[(3S)-1-(methanesulfonyl)pyrrolidin-3-yl]-1H-pyrazol-4-yl}-6-phenylfuro[2,3-d]pyrimidine

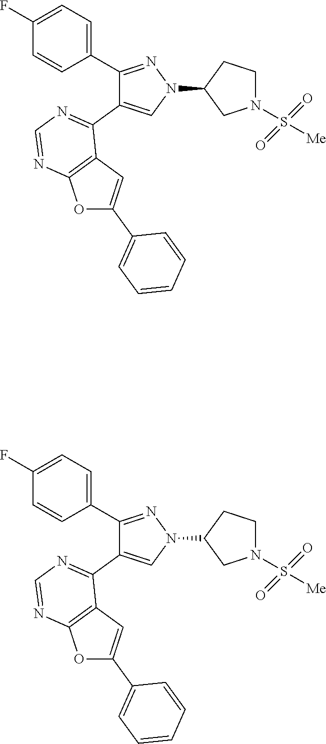

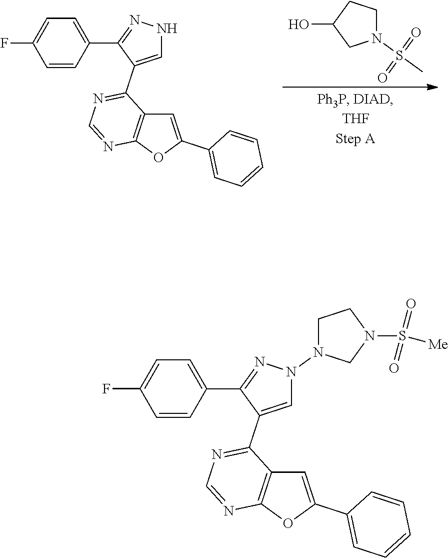

To a stirred solution of 4-[3-(4-fluorophenyl)-1H-pyrazol-4-yl]-6-phenylfuro[2,3-d]pyrimidine (Intermediate N, 200 mg, 0.56 mmol, 1 equiv) and Ph₃P (221 mg, 0.84 mmol, 1.5 equiv) in THF (5.5 mL) were added 1-methanesulfonylpyrrolidin-3-ol (111 mg, 0.67 mmol, 1.2 equiv) and DIAD (227 mg, 1.12 mmol, 2 equiv) at room temperature under nitrogen atmosphere. The resulting mixture was stirred for 2 h at room temperature. The resulting mixture was extracted with EtOAc (3×50 mL). The combined organic layers were washed with brine (3×50 mL), dried over anhydrous Na₂SO₄. After filtration, the filtrate was concentrated under reduced pressure. The crude product was purified by prep-HPLC with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 48% B to 63% B in 8 min, 63% B; Wavelength: 254 nm; RT1 (min): 7.48 to afford Example 89 (115 mg, 41%) as a white solid. The racemic was separated by Chiral-HPLC with the following conditions: Column: CHIRALPAK IG, 2×25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 25% B to 25% B in 27 min; Wavelength: 220/254 nm; RT1 (min): 20.74; RT2 (min): 24.28; Sample Solvent: MEOH:DCM=1:2 (0.1% FA) to afford Example 101 (44.1 mg, 38%) (absolute stereochemistry arbitrarily assigned) as a white solid and Example 102 (44.8 mg, 39%) (absolute stereochemistry arbitrarily assigned) as a white solid.

Example 101. m/z (ESI, +ve ion)=504.30 [M+H]⁺. ¹H NMR (400 MHz, DMSO-d₆) δ 8.78 (s, 1H), 8.70 (s, 1H), 7.93-7.91 (m, 2H), 7.64-7.50 (m, 5H), 7.40 (s, 1H), 7.22-7.18 (m, 2H), 5.24-5.21 (m, 1H), 3.89 (dd, J=10.8, 6.8 Hz, 1H), 3.77 (dd, J=10.8, 4.4 Hz, 1H), 3.79-3.75 (m, 1H), 3.63-3.59 (m, 1H), 2.99 (s, 3H), 2.58-2.51 (m, 2H).

Example 102. m/z (ESI, +ve ion)=504.30 [M+H]⁺. ¹H NMR (400 MHz, DMSO-d₆) δ 8.80 (s, 1H), 8.70 (s, 1H), 7.93-7.91 (m, 2H), 7.64-7.50 (m, 5H), 7.40 (s, 1H), 7.22-7.18 (m, 2H), 5.24-5.22 (m, 1H), 3.89 (dd, J=10.8, 6.8 Hz, 1H), 3.77 (dd, J=10.8, 4.4 Hz, 1H), 3.79-3.75 (m, 1H), 3.63-3.59 (m, 1H), 2.99 (s, 3H), 2.58-2.51 (m, 2H).

### Example 103. N-{2-[3-(4-Fluorophenyl)-4-(6-phenylfuro[2,3-d]pyrimidin-4-yl)-1H-pyrazol-1-yl]ethyl}methanesulfonamide

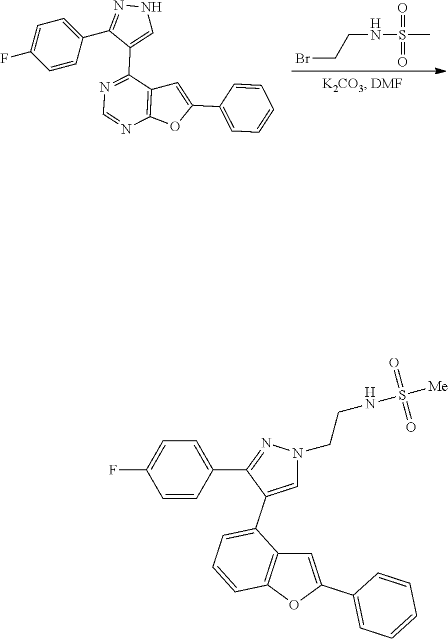

To a stirred mixture of 4-[3-(4-fluorophenyl)-1H-pyrazol-4-yl]-6-phenylfuro[2,3-d]pyrimidine (Intermediate N, 100 mg, 0.281 mmol, 1 equiv) and K₂CO₃ (77.56 mg, 0.562 mmol, 2 equiv) in DMF (5 mL) was added N-(2-bromoethyl)methanesulfonamide (68.04 mg, 0.337 mmol, 1.2 equiv) at room temperature under nitrogen atmosphere. The resulting mixture was stirred for 16 h at room temperature under nitrogen atmosphere then quenched by the addition of water (10 mL) at room temperature. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by prep-HPLC with the following conditions: Column: Xselect CSH C18 OBD Column 30×150 mm 5 μm, Mobile Phase A: water (10 mmol/L NH₄HCO₃), Mobile Phase B: ACN; Flow rate: 60 mL/min; Gradient: 42% B to 42% B in 8 min, 42% B; Wavelength: 254 nm; RT1 (min): 12 to afford Example 103 (32.8 mg, 24%) as a white solid. m/z (ESI, +ve ion)=478.15 [M+H]⁺. ¹H NMR (400 MHz, DMSO-d₆) δ 8.64 (s, 1H), 7.92 (d, J=7.6 Hz, 1H), 7.72-7.46 (m, 6H), 7.45-7.38 (m, 2H), 7.25-7.16 (m, 2H), 4.39 (t, J=6.0 Hz, 2H), 3.55 (t, J=6.0 Hz, 2H), 2.95-2.90 (m, 3H).

### Example 104. 3-[4-(4-Fluorophenyl)-5-(6-phenylfuro[2,3-d]pyrimidin-4-yl)-2H-1,2,3-triazol-2-yl]-1λ6-thietane-1,1-dione

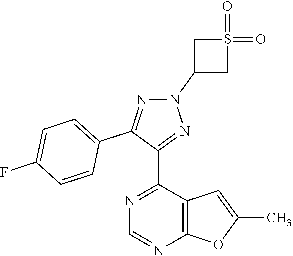 [SMILES: Cc1cc2c(-c3nn(C4CS(=O)(=O)C4)nc3-c3ccc(F)cc3)ncnc2o1]

### Step 1. 4-Bromo-5-(4-fluorophenyl)-2-{[2-(trimethylsilyl)ethoxy]methyl}-1,2,3-triazole

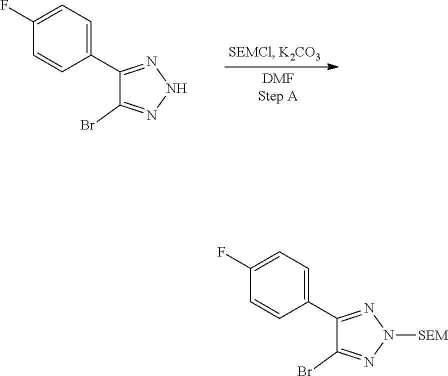 [SMILES: Fc1ccc(-c2n[nH]nc2Br)cc1 ; C[Si](C)(C)CCOCn1nc(Br)c(-c2ccc(F)cc2)n1]

To a stirred solution of 4-bromo-5-(4-fluorophenyl)-2H-1,2,3-triazole (Intermediate M, 1 g, 4.131 mmol, 1 equiv) and K₂CO₃ (1.14 g, 8.262 mmol, 2 equiv) in DMF (20 mL) was added SEMCl (1.03 g, 6.197 mmol, 1.5 equiv) dropwise at room temperature under nitrogen atmosphere. The resulting mixture was stirred for 20 min at room temperature under nitrogen atmosphere then quenched by the addition of water (20 mL) at 0° C. The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with PE:EA (5:1) to afford the title compound (1.2 g, 78%) as a yellow oil. m/z (ESI, +ve ion)=372.10, 374.10 [M+H]⁺.

### Step 2. 4-(4-Fluorophenyl)-5-(4,4,5,5-tetramethyl-1,3,2-dioxaborolan-2-yl)-2-{[2-(trimethylsilyl)ethoxy]methyl}-1,2,3-triazole

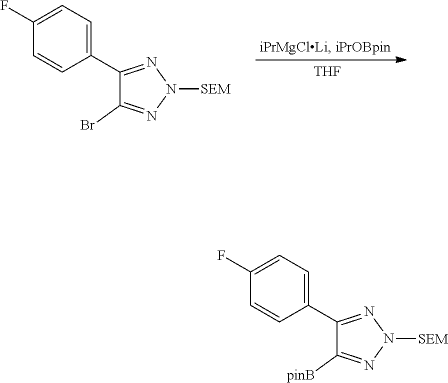

To a stirred mixture of 4-bromo-5-(4-fluorophenyl)-2-{[2-(trimethylsilyl)ethoxy]methyl}-1,2,3-triazole (999 mg, 2.683 mmol, 1 equiv) in THF (20 mL) was added i-PrMgCl—LiCl (1.3 M in THF, 3.10 mL, 4.024 mmol, 1.5 equiv) dropwise at 0° C. under nitrogen atmosphere. The resulting mixture was stirred for 1 h at 0° C. under nitrogen atmosphere. To the above mixture was added 2-isopropoxy-4,4,5,5-tetramethyl-1,3,2-dioxaborolane (748.86 mg, 4.024 mmol, 1.5 equiv) dropwise over 2 min at 0° C. The resulting mixture was stirred for additional 2 h at room temperature. The reaction was quenched by the addition of water (40 mL) at room temperature and extracted with EtOAc (3×50 mL). The combined organic layers were washed with brine (3×20 mL), dried over anhydrous Na₂SO₄. After filtration, the filtrate was concentrated under reduced pressure to afford the title compound (1 g, 89%) as a yellow oil. m/z (ESI, +ve ion)=420.30 [M+H]⁺.

### Step 3. 4-(4-Fluorophenyl)-5-{6-phenylfuro[2,3-d]pyrimidin-4-yl}-2-{[2-(trimethylsilyl)ethoxy]methyl}-1,2,3-triazole

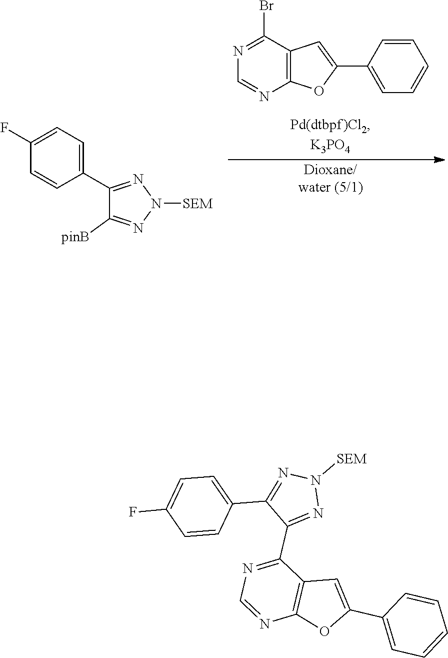

To a stirred mixture of 4-bromo-6-phenylfuro[2,3-d]pyrimidine (Intermediate I, 580 mg, 2.108 mmol, 1 equiv) and 4-(4-fluorophenyl)-5-(4,4,5,5-tetramethyl-1,3,2-dioxaborolan-2-yl)-2-{[2-(trimethylsilyl)ethoxy]methyl}-1,2,3-triazole (1.33 g, 3.162 mmol, 1.5 equiv) in 1,4-dioxane (5 mL) and H₂O (1 mL) were added K₃PO₄ (895.03 mg, 4.216 mmol, 2 equiv) and Pd(DtBPF)Cl₂ (274.82 mg, 0.422 mmol, 0.2 equiv) at room temperature under nitrogen atmosphere. The resulting mixture was stirred for 2 h at 90° C. under nitrogen atmosphere then quenched by the addition of water (40 mL) at room temperature.

The resulting mixture was extracted with EtOAc (3×50 mL). The combined organic layers were washed with brine (3×20 mL), dried over anhydrous Na₂SO₄. After filtration, the filtrate was concentrated under reduced pressure. The residue was purified by silica gel column chromatography, eluted with PE:EA (10:1) to afford the title compound (720 mg, 70%) as a yellow solid. m/z (ESI, +ve ion)=488.20 [M+H]⁺.

### Step 4. 4-(4-Fluorophenyl)-5-{6-phenylfuro[2,3-d]pyrimidin-4-yl}-2H-1,2,3-triazole

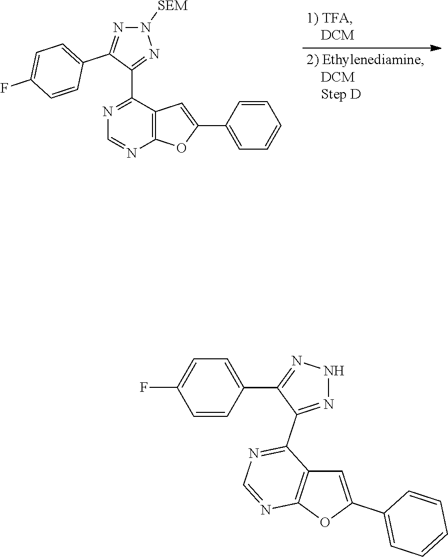 [SMILES: C[Si](C)(C)CCOCn1nc(-c2ccc(F)cc2)c(-c2ncnc3oc(-c4ccccc4)cc23)n1 ; Fc1ccc(-c2n[nH]nc2-c2ncnc3oc(-c4ccccc4)cc23)cc1]

A mixture of 4-(4-fluorophenyl)-5-{6-phenylfuro[2,3-d]pyrimidin-4-yl}-2-{[2-(trimethylsilyl)ethoxy]methyl}-1,2,3-triazole (680 mg, 1.395 mmol, 1 equiv) in DCM (3 mL) and TFA (1 mL) was stirred for overnight at room temperature under nitrogen atmosphere. The resulting mixture was concentrated under reduced pressure. To the above mixture was added DCM (5 mL) and 1,2-ethylenediamine (1 mL) dropwise over 2 min at room temperature. The resulting mixture was stirred for additional 2 h at room temperature then concentrated under reduced pressure. The residue was purified by silica gel column chromatography, eluted with PE:EA (1:1) to afford the title compound (480 mg, 96%) as an off-white solid. m/z (ESI, +ve ion)=358.15 [M+H]⁺.

### Step 5. 3-[4-(4-Fluorophenyl)-5-(6-phenylfuro[2,3-d]pyrimidin-4-yl)-2H-1,2,3-triazol-2-yl]-1λ6-thietane-1,1-dione

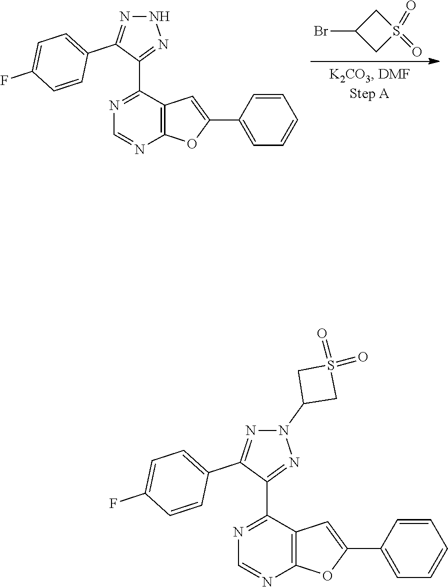 [SMILES: Fc1ccc(-c2n[nH]nc2-c2ncnc3oc(-c4ccccc4)cc23)cc1 ; O=S1(=O)CC(Br)C1 ; O=S1(=O)CC(n2nc(-c3ccc(F)cc3)c(-c3ncnc4oc(-c5ccccc5)cc34)n2)C1]

To a stirred mixture of 4-(4-fluorophenyl)-5-{6-phenylfuro[2,3-d]pyrimidin-4-yl}-2H-1,2,3-triazole (80 mg, 0.224 mmol, 1 equiv) and K₂CO₃ (61.88 mg, 0.448 mmol, 2 equiv) in DMF (1 mL) was added 3-bromo-1,6-thietane-1,1-dione (62.14 mg, 0.336 mmol, 1.5 equiv) at room temperature under nitrogen atmosphere. The resulting mixture was stirred for overnight at 60° C. under nitrogen atmosphere. The resulting mixture was filtered, the filter cake was washed with EtOAc (3×10 mL). The filtrate was concentrated under reduced pressure. The residue was purified by reverse flash chromatography with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 55% B to 70% B in 8 min, 70% B; Wavelength: 254 nm; RT1 (min): 7.95 to afford Example 104 (24.9 mg, 24%) as a white solid. m/z (ESI, +ve ion)=462.10 [M+H]⁺. ¹H-NMR (400 MHz, Chloroform-d) δ 8.93 (s, 1H), 7.99-7.91 (m, 4H), 7.56-7.46 (m, 4H), 7.19-7.15 (m, 2H), 5.71-5.65 (m, 1H), 5.08-5.03 (m, 2H), 4.94-4.88 (m, 2H).

### Example 106. 2-[3-(4-Fluorophenyl)-4-(6-phenylfuro[2,3-d]pyrimidin-4-yl)-1H-pyrazol-1-yl]acetamide

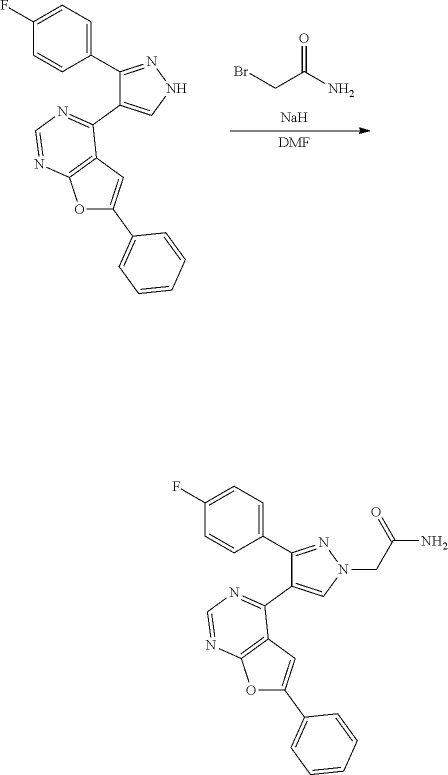

4-[3-(4-Fluorophenyl)-1H-pyrazol-4-yl]-6-phenylfuro[2,3-d]pyrimidine (Intermediate N, 100 mg, 281 μmol) was dissolved in DMF (935 μL) then sodium hydride (8.08 mg, 337 μmol, 1.2 equiv., 60% in dispersion in mineral oil) was added, followed by 2-bromoacetamide (43.5 mg, 309 μmol, 1.1 equiv.) at room temperature. The reaction mixture was stirred at room temperature for 16 hours. The reaction mixture diluted with water and EtOAc. The organic layer was and washed with water, then with brine, dried with Na₂SO₄, filtered, and concentrated under reduced pressure. The residue was purified by prep HPLC (Buchi Pure C850, Phenomenex Luna C18, 10 μm, 250 mm×21.2 mm, eluent: MeCN in water with 10 mM ammonium formate, 20-100%, a gradient elution) to provide the title compound (25.8 mg, 21%) as a solid. ¹H NMR (400 MHz, DMSO-d₆) δ 8.78 (s, 1H), 8.63 (s, 1H), 7.92-7.86 (m, 2H), 7.69 (s, 1H), 7.63-7.53 (m, 4H), 7.53-7.46 (m, 1H), 7.41 (s, 1H), 7.32 (s, 1H), 7.23-7.15 (m, 2H), 4.94 (s, 2H). m/z (ESI, +ve ion)=414.4 [M+H]⁺.

### Example 108. 3-{4-[6-(1-Acetyl-1,2,3,6-tetrahydropyridin-4-yl)furo[2,3-d]pyrimidin-4-yl]-3-(4-fluorophenyl)-1H-pyrazol-1-yl}-1λ6-thietane-1,1-dione

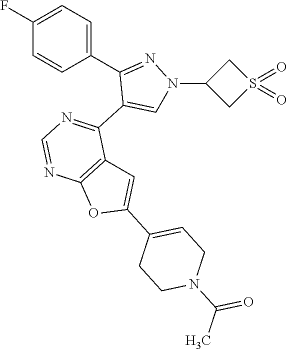 [SMILES: CC(=O)N1CC=C(c2cc3c(-c4cn(C5CS(=O)(=O)C5)nc4-c4ccc(F)cc4)ncnc3o2)CC1]

### Step 1. 1-(4-(4-(3-(4-Fluorophenyl)-1H-pyrazol-4-yl)furo[2,3-d]pyrimidin-6-yl)-5,6-dihydropyridin-1 (2H)-yl)ethanone

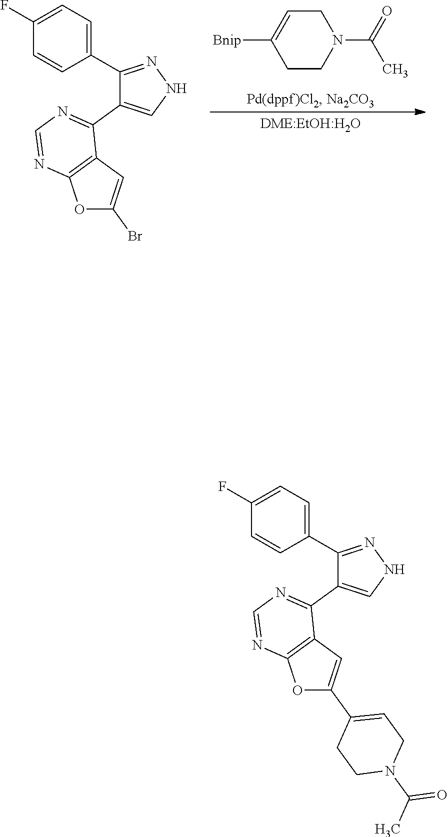

1,1′-Bis(diphenylphosphino)ferrocene dichloropalladium (II) (42.9 mg, 55.7 μmol, 0.1 equiv.) was added to a degassed mixture 6-bromo-4-(3-(4-fluorophenyl)-1H-pyrazol-4-yl)furo[2,3-d]pyrimidine (Intermediate Q, 200 mg, 557 μmol), 1-(4-(4,4,5,5-Tetramethyl-1,3,2-dioxaborolan-2-yl)-5,6-dihydropyridin-1 (2H)-yl)ethanone (154 mg, 613 μmol, 1.1 equiv.), and sodium carbonate (179 mg, 1.67 mmol, 3.0 equiv.) in a mixture of DME (2.60 mL), EtOH (1.30 mL), and H₂O (650 μL). The mixture was degassed with nitrogen for 3 minutes, then heated at 110° C. for 45 minutes in a microwave. Additional 1,1′-bis(diphenylphosphino)ferrocene dichloropalladium (II) (21.4 mg, 27.8 μmol, 0.05 equiv.) was added and the mixture was heated at 115° C. for 1.5 h in a microwave reactor. The reaction mixture was cooled to room temperature then diluted with EtOAc. The solvent was removed under removed pressure. The residue was purified by reverse phase chromatography (C18 column, MeCN in water with 10 mM ammonium formate, 5-35%, a gradient elution) to provide the title compound (78.3 mg, 35%) as a solid. m/z (ESI, +ve ion)=404.3 [M+H]⁺.

### Step 2. 3-{4-[6-(1-Acetyl-1,2,3,6-tetrahydropyridin-4-yl)furo[2,3-d]pyrimidin-4-yl]-3-(4-fluorophenyl)-1H-pyrazol-1-yl}-1λ6-thietane-1,1-dione

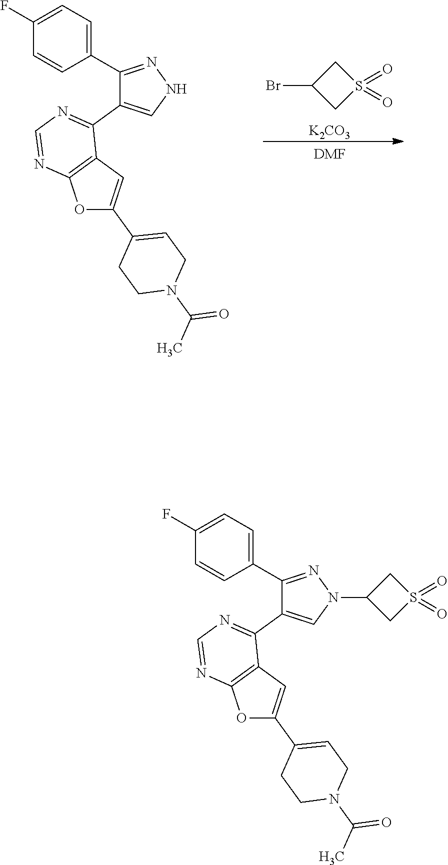

A mixture of 1-(4-(4-(3-(4-fluorophenyl)-1H-pyrazol-4-yl)furo[2,3-d]pyrimidin-6-yl)-5,6-dihydropyridin-1 (2H)-yl)ethanone (75.0 mg, 186 μmol) and potassium carbonate (52.4 mg, 372 μmol, 2.0 equiv.) in DMF (1.50 mL) was stirred at room temperature for 5 minutes, then 3-bromothietane 1,1-dioxide (54.3 mg, 279 μmol, 1.5 equiv.) was added. The mixture was stirred for 16 h at room temperature. The mixture was diluted with H₂O (10 mL), EtOAc (20 mL) and brine (25 mL). The aqueous layer was extracted with EtOAc (3×25 mL) and the combined organic layers were washed with H₂O (15 mL), brine (15 mL), dried over Na₂SO₄, filtered and concentrated. The residue was purified by column chromatography (MeOH in DCM, 0-4%, a gradient elution) to provide the title compound (35.5 mg, 38%) as a solid. ¹H NMR (400 MHz, DMSO-d₆) δ 8.78 (s, 1H), 8.71 (s, 1H), 7.59 (dd, J=7.9, 5.7 Hz, 2H), 7.25-7.17 (m, 2H), 6.67 (d, J=12.9 Hz, 1H), 6.62-6.57 (m, 1H), 5.56-5.43 (m, 1H), 4.95-4.77 (m, 4H), 4.25 (app s, 1H), 4.19 (app s, 1H) 3.65 (dt, J=9.1, 5.6 Hz, 2H), 2.43 (app s, 1H), 2.33 (app s, 1H), 2.08 and 2.05 (s, 3H). m/z (ESI, +ve ion)=508.3 [M+H]⁺.

### Example 109. 1-[3-(2-Fluorophenyl)-4-(6-phenylfuro[2,3-d]pyrimidin-4-yl)-1H-pyrazol-1-yl]-2-methylpropan-2-ol

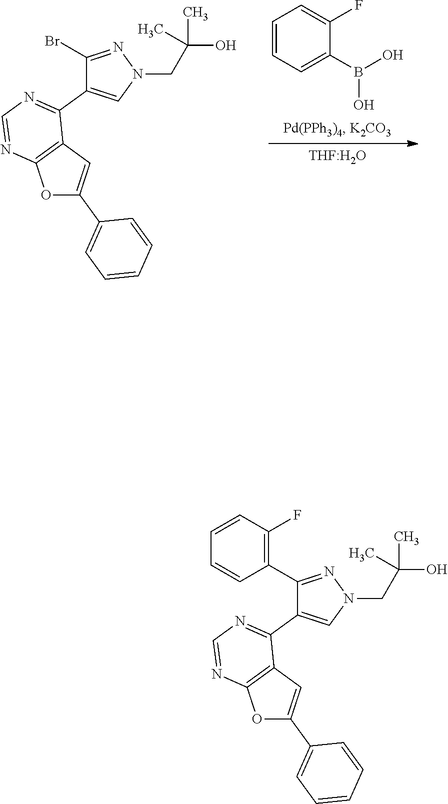 [SMILES: CC(C)(O)Cn1cc(-c2ncnc3oc(-c4ccccc4)cc23)c(Br)n1 ; OB(O)c1ccccc1F ; "THF:H2O" ; CC(C)(O)Cn1cc(-c2ncnc3oc(-c4ccccc4)cc23)c(-c2ccccc2F)n1]

1-(3-Bromo-4-(6-phenylfuro[2,3-d]pyrimidin-4-yl)-1H-pyrazol-1-yl)-2-methylpropan-2-ol (Intermediate P, 90.0 mg, 218 μmol), 2-fluorophenylboronic acid (60.9 mg, 427 μmol, 2.0 equiv.), tetrakis(triphenylphosphine)palladium(0) (25.7 mg, 21.8 μmol, 0.1 equiv.) and potassium carbonate (92.1 mg, 653 μmol, 3.0 equiv.) was purged with nitrogen and evacuated three time, then THF (1.45 mL) and H₂O (726 μL) were then added. The mixture was purged with nitrogen and evacuated three time, and stirred at 80° C. for 16 h. The mixture was cooled to room temperature, diluted with EtOAc, and then filtered. The filtrate was then washed with brine, dried over Na₂SO₄, filtered, and concentrated under reduced pressure. The residue was purified by reverse phase chromatography (C18 column, MeCN in water with 10 mM ammonium formate, 5-70%, a gradient elution) to provide the title compound (56.3 mg, 60%) as a solid. ¹H NMR (400 MHz, DMSO-d₆) δ 8.67 (s, 1H), 8.65 (s, 1H), 7.91-7.88 (m, 2H), 7.61-7.54 (m, 3H), 7.51-7.42 (m, 2H), 7.40 (s, 1H), 7.32 (td, J=7.5, 1.0 Hz, 1H), 7.19-7.14 (m, 1H), 4.88 (s, 1H), 4.22 (s, 2H), 1.20 (s, 6H). m/z (ESI, +ve ion)=429.3 [M+H]⁺.

### Example 110. 2-Methyl-1-[3-phenyl-4-(6-phenylfuro[2,3-d]pyrimidin-4-yl)-1H-pyrazol-1-yl]propan-2-ol

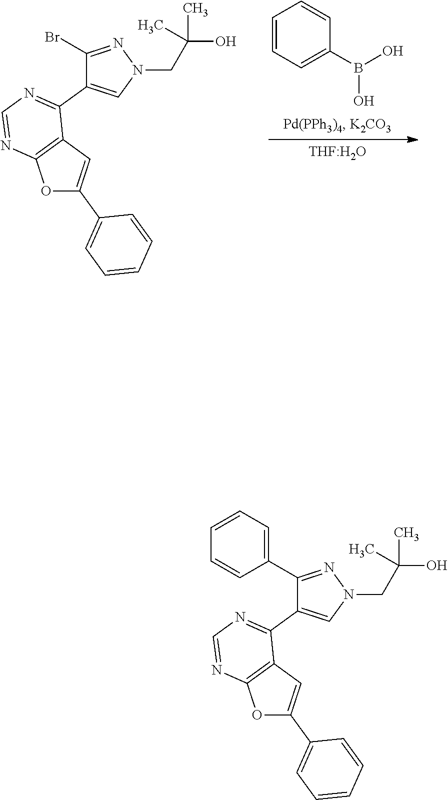 [SMILES: CC(C)(O)Cn1cc(-c2ncnc3oc(-c4ccccc4)cc23)c(Br)n1 ; OB(O)c1ccccc1 ; "THF:H2O" ; CC(C)(O)Cn1cc(-c2ncnc3oc(-c4ccccc4)cc23)c(-c2ccccc2)n1]

1-(3-Bromo-4-(6-phenylfuro[2,3-d]pyrimidin-4-yl)-1H-pyrazol-1-yl)-2-methylpropan-2-ol (Intermediate P, 87.0 mg, 158 μmol), phenylboronic acid (38.5 mg, 309 μmol, 2.0 equiv.), tetrakis(triphenylphosphine)palladium(0) (18.6 mg, 15.8 μmol, 0.1 equiv.) and potassium carbonate (66.8 mg, 474 μmol, 3.0 equiv.) were added under nitrogen, then THF (1.05 mL) and H₂O (526 μL) were added. The mixture was purged with nitrogen and evacuated three times then heated at 80° C. for 16 h. The mixture was cooled to room temperature, diluted with EtOAc, and then filtered. The filtrate was then washed with brine, dried over Na₂SO₄, filtered, and concentrated under reduced pressure. The residue was purified by reverse phase chromatography (C18 column, MeCN in water with 10 mM ammonium formate (5-70%, a gradient elution) to provide the title compound (33.0 mg, 51%) as a solid. ¹H NMR (400 MHz, DMSO-d₆) δ 8.79 (s, 1H), 8.48 (s, 1H), 7.84-7.79 (m, 2H), 7.57-7.51 (m, 4H), 7.51-7.45 (m, 1H), 7.40-7.34 (m, 3H), 7.06 (s, 1H), 4.86 (s, 1H), 4.20 (s, 2H), 1.20 (s, 6H). m/z (ESI, +ve ion)=411.3 [M+H]⁺.

### Example 111. 2-[3-(4-Fluorophenyl)-4-(6-phenylfuro[2,3-d]pyrimidin-4-yl)-1H-pyrazol-1-yl]ethane-1-sulfonamide

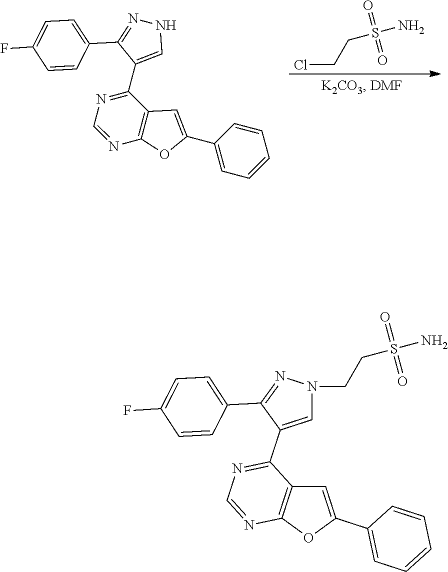 [SMILES: Fc1ccc(-c2n[nH]cc2-c2ncnc3oc(-c4ccccc4)cc23)cc1 ; NS(=O)(=O)CCCl ; NS(=O)(=O)CCn1cc(-c2ncnc3oc(-c4ccccc4)cc23)c(-c2ccc(F)cc2)n1]

To a stirred solution of 4-[3-(4-Fluorophenyl)-1H-pyrazol-4-yl]-6-phenylfuro[2,3-d]pyrimidine (Intermediate N, 100 mg, 0.28 mmol, 1 equiv) and K₂CO₃ (78 mg, 0.562 mmol, 2 equiv) in DMF (1.5 mL) was added 2-chloroethanesulfonamide (48.35 mg, 0.337 mmol, 1.2 equiv) at room temperature under nitrogen atmosphere. The resulting mixture was stirred overnight at room temperature under nitrogen atmosphere then quenched with water at room temperature. The resulting mixture was extracted with EtOAc (3×10 mL). The combined organic layers were washed with brine (10 mL), dried over anhydrous Na₂SO₄. After filtration, the filtrate was concentrated under reduced pressure. The crude product (100 mg) was purified by prep-HPLC with the following conditions: Column: XBridge Shield RP18 OBD Column, 19×250 mm, 10 μm; Mobile Phase A: water (10 mmol/L NH₄HCO₃), Mobile Phase B: MeOH-HPLC; Flow rate: 25 mL/min; Gradient: 30% B to 60% B in 10 min, 60% B; Wavelength: 254 nm; RT1 (min): 9 to afford Example 111 (52.6 mg, 40%) as a white solid. m/z (ESI, +ve ion)=464.20 [M+H]⁺. ¹H NMR (400 MHz, DMSO-d₆) δ 8.77 (s, 1H), 8.72 (s, 1H), 7.92-7.90 (m, 2H), 7.65-7.62 (m, 2H), 7.59-7.55 (m, 2H), 7.52-7.48 (m, 1H), 7.44 (s, 1H), 7.22-7.17 (m, 4H), 4.72-4.68 (m, 2H), 3.74-3.71 (m, 2H).

### Example 116. 4-[3-(4-Fluorophenyl)-4-(6-phenylfuro[2,3-d]pyrimidin-4-yl)-1H-pyrazol-1-yl]butanenitrile

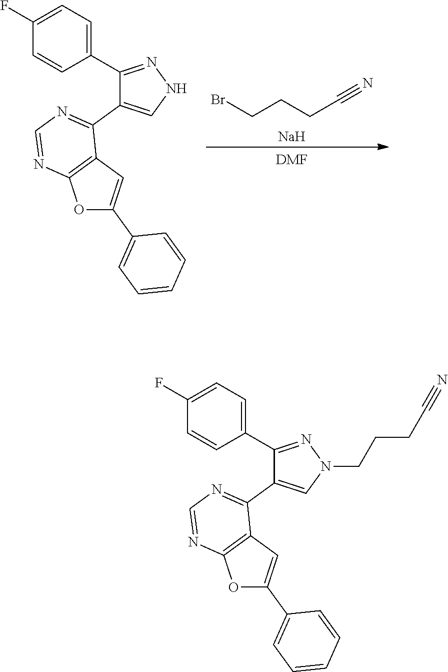

4-[3-(4-Fluorophenyl)-1H-pyrazol-4-yl]-6-phenylfuro[2,3-d]pyrimidine (Intermediate N, 100 mg, 281 μmol) was dissolved in DMF (935 μL) then sodium hydride (8.08 mg, 337 μmol, 1.2 equiv., 60% in dispersion in mineral oil) was added, followed by 4-bromobutyronitrile (34.2 μL, 337 μmol, 1.2 equiv.) at room temperature. The reaction mixture was stirred at room temperature for 16 hours. The reaction mixture was diluted with water and EtOAc. The organic layer was washed with water, then with brine, dried with Na₂SO₄, filtered, and concentrated under reduced pressure. The residue was purified by column chromatography (EtOAc in hexanes, of 0-100%, a gradient elution) to provide the title compound (23.4 mg, 19%) as a solid. ¹H NMR (400 MHz, DMSO-d₆) δ 8.77 (s, 1H), 8.68 (s, 1H), 7.95-7.88 (m, 2H), 7.65-7.53 (m, 4H), 7.53-7.46 (m, 1H), 7.44 (s, 1H), 7.23-7.16 (m, 2H), 4.37 (t, J=6.9H, 2 Hz), 2.64 (t, J=7.1 Hz, 2H), 2.26 (p, J=7.0 Hz, 2H). m/z (ESI, +ve ion)=424.4 [M+H]⁺.

### Example 118. 3-[3-(4-Fluorophenyl)-4-(6-phenylfuro[2,3-d]pyrimidin-4-yl)-1H-pyrazol-1-yl]propanamide

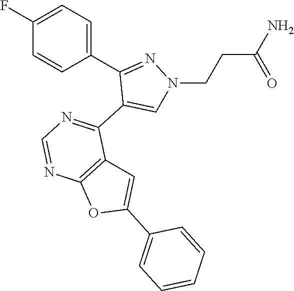 [SMILES: NC(=O)CCn1cc(-c2ncnc3oc(-c4ccccc4)cc23)c(-c2ccc(F)cc2)n1]

### Step A. ethyl 3-(3-(4-Fluorophenyl)-4-(6-phenylfuro[2,3-d]pyrimidin-4-yl)-1H-pyrazol-1-yl)propanoate

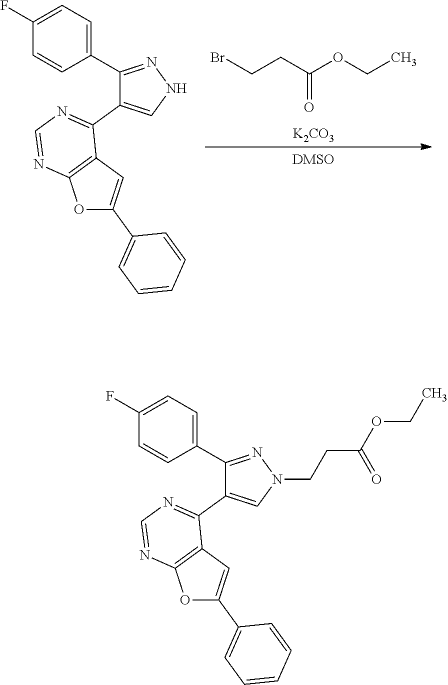

7-(3-(4-fluorophenyl)-1H-pyrazol-4-yl)-2-phenyloxazolo[5,4-d]pyrimidine (Intermediate N, 200 mg, 561 μmol) and potassium carbonate (198 mg, 1.40 mmol, 2.5 equiv.) were suspended in DMSO (2.81 mL) at room temperature. After 5 minutes, ethyl 3-bromopropionate (87.6 μL, 673 μmol, 1.2 equiv.) was added dropwise and the reaction mixture was stirred at 25° C. for 16 hours. Additional ethyl 3-bromopropionate (36.5 μL, 281 μmol, 0.5 equiv.) was added and the mixture was further stirred for 20 h at 25° C. H₂O (10 mL) and EtOAc (10 mL) were added and the layers were partitioned. The aqueous layer was extracted with additional EtOAc (3×20 mL). The combined organic layers were washed with a saturated solution of Na₂S₂O₃ (10 mL), H₂O (10 mL), brine (10 mL), dried over Na₂SO₄, filtered, and concentrated to provide the title compound (208 mg, 81%) as an oil. m/z (ESI, +ve ion)=457.4 [M+H]⁺.

### Step 2. 3-(3-(4-Fluorophenyl)-4-(6-phenylfuro[2,3-d]pyrimidin-4-yl)-1H-pyrazol-1-yl)propanoic acid

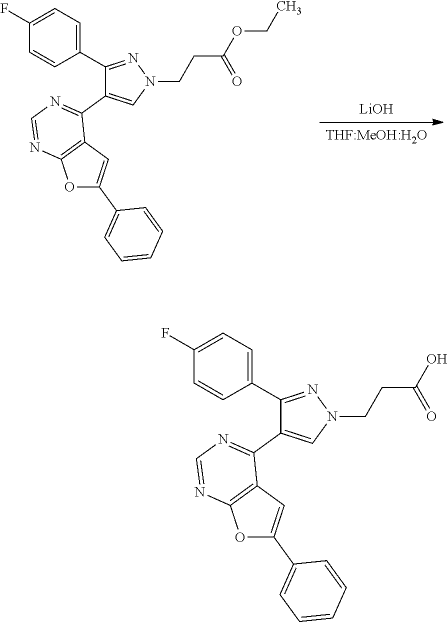 [SMILES: CCOC(=O)CCn1cc(-c2ncnc3oc(-c4ccccc4)cc23)c(-c2ccc(F)cc2)n1 ; "LiOH" ; "THF:MeOH:H2O" ; O=C(O)CCn1cc(-c2ncnc3oc(-c4ccccc4)cc23)c(-c2ccc(F)cc2)n1]

Ethyl 3-(3-(4-fluorophenyl)-4-(6-phenylfuro[2,3-d]pyrimidin-4-yl)-1H-pyrazol-1-yl)propanoate (208 mg, 433 μmol) was dissolved in a mixture of THF (3.00 mL), MeOH (1.50 mL) and water (750 μL). Lithium hydroxide (106 mg, 4.33 mmol, 10 equiv.) was added and the mixture was stirred at room temperature for 72 h. The mixture was diluted with water (10 mL) and EtOAc (10 mL). The layers were separated, and the aqueous layer was washed with EtOAc (2×10 mL). The aqueous layer was adjusted to pH ˜ 4-5 using an aqueous 1 M HCl solution and extracted with EtOAc (3×15 mL). The combined organic layers were dried over Na₂SO₄, filtered, and concentrated under reduced pressure to provide the title compound (155 mg, 84%) as a solid. m/z (ESI, +ve ion)=429.3 [M+H]⁺.

### Step 3. 3-[3-(4-Fluorophenyl)-4-(6-phenylfuro[2,3-d]pyrimidin-4-yl)-1H-pyrazol-1-yl]propanamide

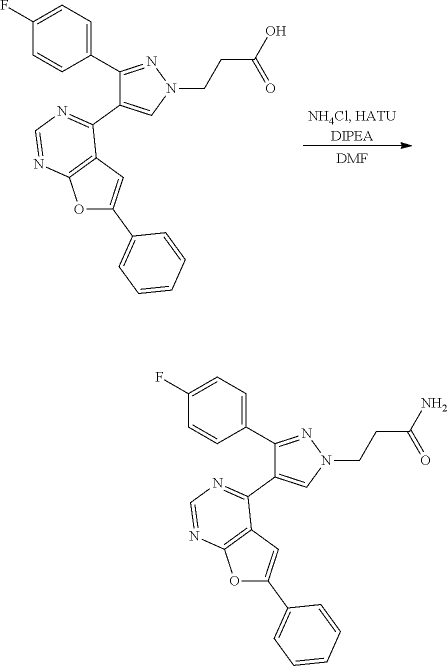

N,N-diisopropylethylamine (64.0 μL, 368 μmol, 2.5 equiv.) and ammonium chloride (31.5 mg, 588 μmol, 4.0 equiv.) were added to a solution of ethyl 3-(3-(4-fluorophenyl)-4-(6-phenylfuro[2,3-d]pyrimidin-4-yl)-1H-pyrazol-1-yl)propanoate (63.0 mg, 147 μmol) in DMF (735 μL) at room temperature. Then, HATU (84.7 mg, 221 μmol, 1.5 equiv.) was added and reaction mixture was stirred at room temperature for 1 h. The mixture was directly purified by reverse phase chromatography (MeCN in water with 10 mM ammonium formate, 10-100%, a gradient elution) to provide the title compound (13.3 mg, 21%) as a solid. ¹H NMR (400 MHz, DMSO-d₆) δ 8.77 (s, 1H), 8.59 (s, 1H), 7.91 (d, J=7.4 Hz, 2H), 7.64-7.53 (m, 4H), 7.53-7.46 (m, 2H), 7.40 (s, 1H), 7.19 (t, J=8.8 Hz, 2H), 6.99 (s, 1H), 4.48 (t, J=6.9 Hz, 2H), 2.80 (t, J=7.0 Hz, 2H). m/z (ESI, +ve ion)=469.4 [M+H]⁺.

### Example 119. 4-[1-(2,2-Difluoroethyl)-3-(4-fluorophenyl)-1H-pyrazol-4-yl]-6-phenylfuro[2,3-d]pyrimidine

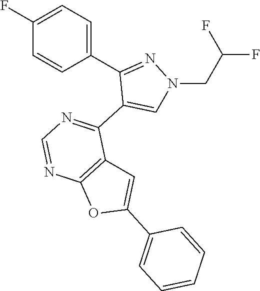 [SMILES: Fc1ccc(-c2nn(CC(F)F)cc2-c2ncnc3oc(-c4ccccc4)cc23)cc1]

### Step 1. 2,2-Difluoroethyl 4-methylbenzenesulfonate

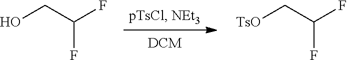

p-toluenesulfonyl chloride (1.42 g, 7.31 mmol, 1.2 equiv.) was added to a mixture of triethylamine (1.72 mL, 12.2 mmol, 2.0 equiv.) and 2,2-difluoroethanol (386 μL, 6.09 mmol) in DCM (25.4 mL). The mixture was stirred for 15 h at room temperature then diluted with water (30 mL). The aqueous layer was extracted with DCM (3×30 mL). The combined organic layers were washed with brine and concentrated. The residue was purified by column chromatography (EtOAc in hexanes, 0-20%, a gradient elution) to provide the title compound (1.30 g, 90%) as an oil. ¹H NMR (400 MHz, CDCl₃) δ 7.81 (d, J=8.2 Hz, 2H), 7.38 (d, J=8.1 Hz, 2H), 5.92 (tt, J=54.6, 4.1 Hz, 1H), 4.17 (td, J=12.7, 4.1 Hz, 2H), 2.47 (s, 3H).

### Step 2. 4-[1-(2,2-Difluoroethyl)-3-(4-fluorophenyl)-1H-pyrazol-4-yl]-6-phenylfuro[2,3-d]pyrimidine

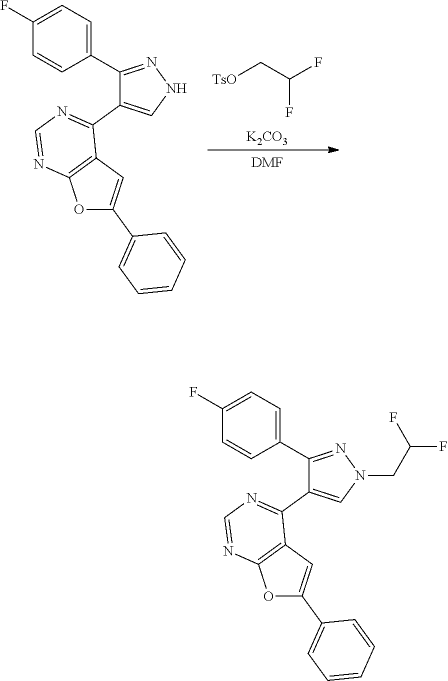

Potassium carbonate (79.1 mg, 561 μmol, 2.0 equiv.) was added to a mixture of 4-[3-(4-fluorophenyl)-1H-pyrazol-4-yl]-6-phenylfuro[2,3-d]pyrimidine (Intermediate N, 100 mg, 281 μmol) and 2,2-difluoroethyl 4-methylbenzenesulfonate (72.9 mg, 309 μmol, 1.1 equiv.) in DMF (2.80 mL). The mixture was stirred for 15 h at room temperature, then at 15 h at 60° C. The mixture was diluted with water (20 mL) and extracted with EtOAc (3×10 mL). The combined organic layers were washed with water, brine, then dried (Na₂SO₄), filtered and concentrated. The crude was purified by column chromatography (EtOAc in hexanes, 0-50%, a gradient elution) to provide (25.0 mg, 21%) as a solid. ¹H NMR (400 MHz, DMSO-d₆) δ 8.81 (s, 1H), 8.70 (s, 1H), 7.94-7.86 (m, 2H), 7.64-7.53 (m, 4H), 7.53-7.46 (m, 1H), 7.30 (s, 1H), 7.21 (t, J=8.9 Hz, 2H), 6.57 (tt, J=54.8, 3.7 Hz, 1H), 4.81 (td, J=14.8, 3.7 Hz, 2H). m/z (ESI, +ve ion)=421.4 [M+H]⁺.

### Example 120. (1s,3s)-3-(3-(4-Fluorophenyl)-4-(6-phenylfuro[2,3-d]pyrimidin-4-yl)-1H-pyrazol-1-yl)cyclobutan-1-ol

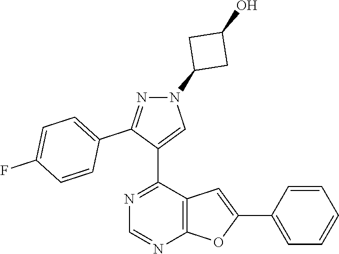 [SMILES: O[C@H]1C[C@@H](n2cc(-c3ncnc4oc(-c5ccccc5)cc34)c(-c3ccc(F)cc3)n2)C1]

### Step 1. 3-(4-Fluorophenyl)-4-{6-phenylfuro[2,3-d]pyrimidin-4-yl}-1-[(1s,3s)-3-(benzyloxy)cyclobutyl]pyrazole and 3-(4-fluorophenyl)-4-{6-phenylfuro[2,3-d]pyrimidin-4-yl}-1-[(1s,3s)-3-(benzyloxy)cyclobutyl]pyrazole

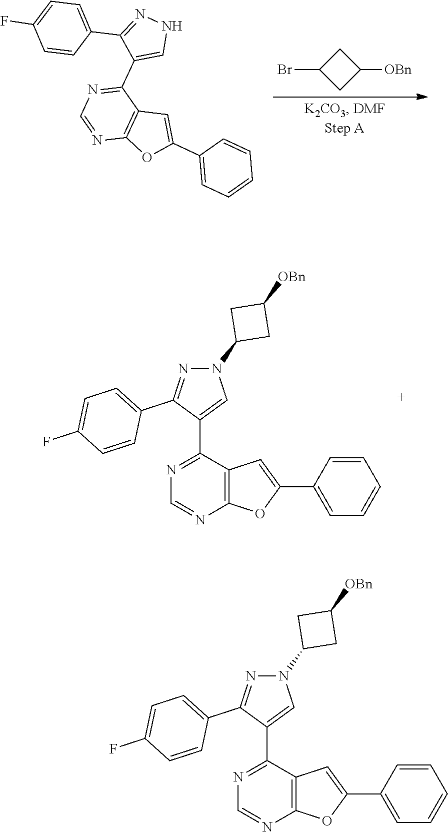

To a stirred mixture of 4-[3-(4-fluorophenyl)-1H-pyrazol-4-yl]-6-phenylfuro[2,3-d]pyrimidine (Intermediate N, 100 mg, 0.281 mmol, 1 equiv) and K₂CO₃ (77.56 mg, 0.562 mmol, 2 equiv) in DMF (1 mL) was added [(3-bromocyclobutoxy)methyl]benzene (101.50 mg, 0.422 mmol, 1.5 equiv) dropwise at room temperature under nitrogen atmosphere. The resulting mixture was stirred for overnight at 60° C. under nitrogen atmosphere then cooled down to room temperature. The resulting mixture was diluted with water (5 mL) and extracted with EtOAc (3×8 mL). The combined organic layers were washed with brine (2×5 mL), dried over anhydrous Na₂SO₄. After filtration, the filtrate was concentrated under reduced pressure. The residue was purified by silica gel column chromatography, eluted with PE:EA (4:1) to afford the trans compound (60 mg, 41%) and the cis compound (60 mg, 41%) as a white solid. m/z (ESI, +ve ion)=517.30 [M+H]⁺.

### Step 2. (1s,3s)-3-(3-(4-Fluorophenyl)-4-(6-phenylfuro[2,3-d]pyrimidin-4-yl)-1H-pyrazol-1-yl)cyclobutan-1-ol

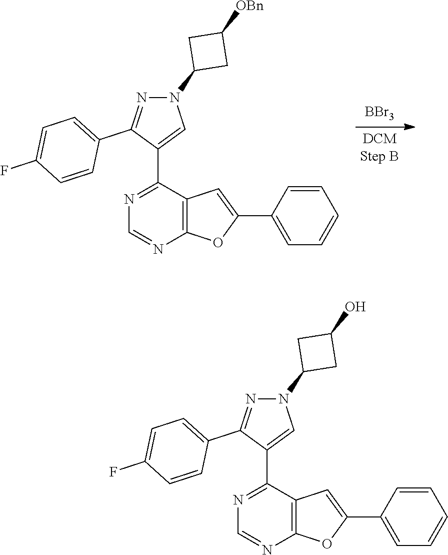 [SMILES: Fc1ccc(-c2nn([C@H]3C[C@@H](OCc4ccccc4)C3)cc2-c2ncnc3oc(-c4ccccc4)cc23)cc1 ; "BBr3" ; "DCMStep B" ; O[C@H]1C[C@@H](n2cc(-c3ncnc4oc(-c5ccccc5)cc34)c(-c3ccc(F)cc3)n2)C1]

To a stirred mixture of 3-(4-fluorophenyl)-4-{6-phenylfuro[2,3-d]pyrimidin-4-yl}-1-[(1s,3s)-3-(benzyloxy)cyclobutyl]pyrazole (60 mg, 0.116 mmol, 1 equiv) in DCM (1 mL) was added BBr₃ (107.66 mg, 0.429 mmol, 3.7 equiv) dropwise at 0° C. under nitrogen atmosphere. The resulting mixture was stirred for 2 h at room temperature under nitrogen atmosphere then quenched by the addition of water (5 mL) at room temperature. The resulting mixture was extracted with EtOAc (3×8 mL). The combined organic layers were washed with brine (10 mL), dried over anhydrous Na₂SO₄. After filtration, the filtrate was concentrated under reduced pressure. The residue was purified by reverse flash chromatography with the following conditions: Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 45% B to 52% B in 8 min, 52% B; Wavelength: 254 nm; RT1 (min): 8.18 to afford Example 120 (26.8 mg, 53%) as a light green solid. m/z (ESI, +ve ion)=427.20 [M+H]⁺. ¹H-NMR (400 MHz, DMSO-d₆) δ 8.77 (s, 1H), 8.63 (s, 1H), 7.91 (d, J=7.2 Hz, 2H), 7.62-7.55 (m, 4H), 7.51-7.47 (m, 1H), 7.39 (s, 1H), 7.22-7.17 (m, 2H), 5.36 (d, J=6.5 Hz, 1H), 4.58-4.50 (m, 1H), 4.07-4.02 (m, 1H), 2.86-2.81 (m, 2H), 2.57-2.53 (m, 2H).

### Example 121. (1r,3r)-3-(3-(4-Fluorophenyl)-4-(6-phenylfuro[2,3-d]pyrimidin-4-yl)-1H-pyrazol-1-yl)cyclobutan-1-ol

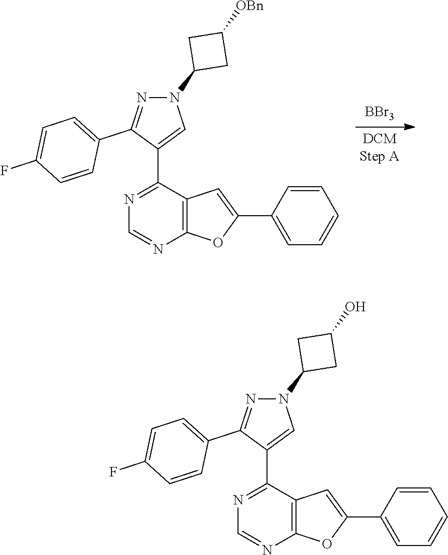 [SMILES: Fc1ccc(-c2nn([C@H]3C[C@H](OCc4ccccc4)C3)cc2-c2ncnc3oc(-c4ccccc4)cc23)cc1 ; "BBr3" ; "DCMStep A" ; O[C@H]1C[C@H](n2cc(-c3ncnc4oc(-c5ccccc5)cc34)c(-c3ccc(F)cc3)n2)C1]

To a stirred mixture of 3-(4-fluorophenyl)-4-{6-phenylfuro[2,3-d]pyrimidin-4-yl}-1-[(1r,3r)-3-(benzyloxy)cyclobutyl]pyrazole (60 mg, 0.116 mmol, 1 equiv) in DCM (1 mL) was added BBr₃ (107.66 mg, 0.429 mmol, 3.7 equiv) dropwise at 0° C. under nitrogen atmosphere. The resulting mixture was stirred for 2 h at room temperature under nitrogen atmosphere then quenched by the addition of water (5 mL) at room temperature. The resulting mixture was extracted with EtOAc (3×8 mL). The combined organic layers were washed with brine (2×5 mL), dried over anhydrous Na₂SO₄. After filtration, the filtrate was concentrated under reduced pressure. The residue was purified by prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 34% B to 44% B in 8 min, 44% B; Wavelength: 254 nm; RT1 (min): 7 to afford Example 121 (35.9 mg, 72%) as a light green solid. m/z (ESI, +ve ion)=427.25 [M+H]⁺. ¹H-NMR (400 MHz, DMSO-d₆) δ 8.77 (s, 1H), 8.64 (s, 1H), 7.94-7.92 (m, 2H), 7.64-7.47 (m, 5H), 7.43 (s, 1H), 7.21-7.17 (m, 2H), 5.28 (d, J=4.8 Hz, 1H), 5.15-5.11 (m, 1H), 4.57-4.54 (m, 1H), 2.86-2.81 (m, 2H), 2.48-2.43 (m, 2H).

### Example 125. (R)-(3R)-3-{[3-(4-Fluorophenyl)-4-(6-phenylfuro[2,3-d]pyrimidin-4-yl)-1H-pyrazol-1-yl]methyl}-16-thiane-1,1-dione

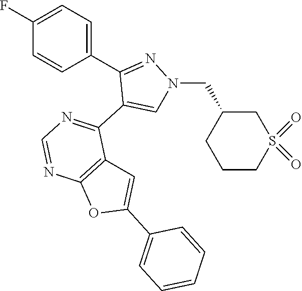 [SMILES: O=S1(=O)CCC[C@H](Cn2cc(-c3ncnc4oc(-c5ccccc5)cc34)c(-c3ccc(F)cc3)n2)C1]

### Example 126. (S)-(3S)-3-{[3-(4-Fluorophenyl)-4-(6-phenylfuro[2,3-d]pyrimidin-4-yl)-1H-pyrazol-1-yl]methyl}-1λ6-thiane-1,1-dione

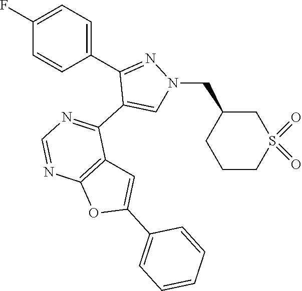 [SMILES: O=S1(=O)CCC[C@@H](Cn2cc(-c3ncnc4oc(-c5ccccc5)cc34)c(-c3ccc(F)cc3)n2)C1]

### Step A. 3-{[3-(4-Fluorophenyl)-4-(6-phenylfuro[2,3-d]pyrimidin-4-yl)-1H-pyrazol-1-yl]methyl}-1λ6-thiane-1,1-dione

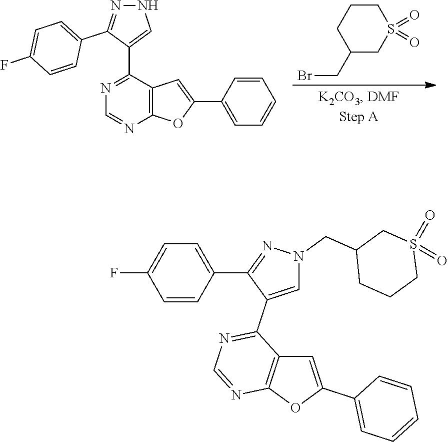

To a stirred mixture of 4-[3-(4-fluorophenyl)-1H-pyrazol-4-yl]-6-phenylfuro[2,3-d]pyrimidine (Intermediate N, 200 mg, 0.561 mmol, 1 equiv) and K₂CO₃ (155.13 mg, 1.122 mmol, 2 equiv) in DMF (2 mL) was added 3-(bromomethyl)-1λ6-thiane-1,1-dione (152.96 mg, 0.673 mmol, 1.2 equiv) at room temperature under nitrogen atmosphere. The resulting mixture was stirred for 2 h at 60° C. under nitrogen atmosphere then cooled down to room temperature. The resulting mixture was filtered, the filter cake was washed with EtOAc (3×10 mL). The filtrate was concentrated under reduced pressure. The residue was purified by prep-HPLC with the following conditions: Column: XBridge Shield RP18 OBD Column, 19×250 mm, 10 m; Mobile Phase A: water (10 mmol/L NH₄HCO₃), Mobile Phase B: ACN; Flow rate: 25 mL/min; Gradient: 56% B to 66% B in 8 min, 66% B; Wavelength: 254 nm; RT1 (min): 7.2 to afford Example 115 (80 mg, 28%) as a white solid. The racemic was separated by Chiral-HPLC with the following conditions: Column: CHIRAL ART Cellulose-SC, 2×25 cm, 5 μm; Mobile Phase A: Hex (0.5% 2M NH₃-MeOH)-HPLC, Mobile Phase B: MeOH:DCM=1:1-HPLC; Flow rate: 20 mL/min; Gradient: 40% B to 40% B in 14 min; Wavelength: 220/254 nm; RT1 (min): 11.75; RT2 (min): 13.37; Sample Solvent: MeOH:DCM=1:1-HPLC to give Example 125 (first peak, 27.1 mg, 30%) (absolute stereochemistry arbitrarily assigned) as a white solid and Example 126 (second peak, 30.6 mg, 34%) (absolute stereochemistry arbitrarily assigned) as a white solid.

Example 125. m/z (ESI, +ve ion)=503.25 [M+H]⁺. ¹H-NMR (400 MHz, Chloroform-d) δ 8.94 (s, 1H), 8.13 (s, 1H), 7.67-7.64 (m, 2H), 7.56-7.52 (m, 2H), 7.48-7.42 (m, 3H), 7.13-7.08 (m, 2H), 6.11 (s, 1H), 4.32-4.31 (m, 2H), 3.21-3.18 (m, 1H), 3.13-3.08 (m, 1H), 2.99-2.91 (m, 3H), 2.24-2.15 (m, 2H), 2.01 (d, J=16 Hz, 1H), 1.43-1.40 (m, 1H).

Example 126. m/z (ESI, +ve ion)=503.30 [M+H]⁺. ¹H-NMR (400 MHz, Chloroform-d) δ 8.94 (s, 1H), 8.13 (s, 1H), 7.67-7.64 (m, 2H), 7.56-7.52 (m, 2H), 7.48-7.42 (m, 3H), 7.13-7.08 (m, 2H), 6.11 (s, 1H), 4.32-4.31 (m, 2H), 3.21-3.18 (m, 1H), 3.13-3.08 (m, 1H), 2.99-2.91 (m, 3H), 2.24-2.15 (m, 2H), 2.01 (d, J=16 Hz, 1H), 1.43-1.40 (m, 1H).

### Example 127. 3-[3-(4-Fluorophenyl)-4-(8-phenyl-9H-purin-6-yl)-1H-pyrazol-1-yl]-1λ6-thietane-1,1-dione

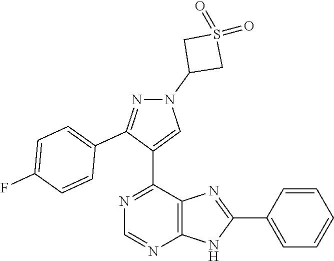 [SMILES: O=S1(=O)CC(n2cc(-c3ncnc4[nH]c(-c5ccccc5)nc34)c(-c3ccc(F)cc3)n2)C1]

### Step 1. 3-[3-(4-Fluorophenyl)-4-{9-[(4-methoxyphenyl)methyl]-8-phenylpurin-6-yl}pyrazol-1-yl]-16-thietane-1,1-dione

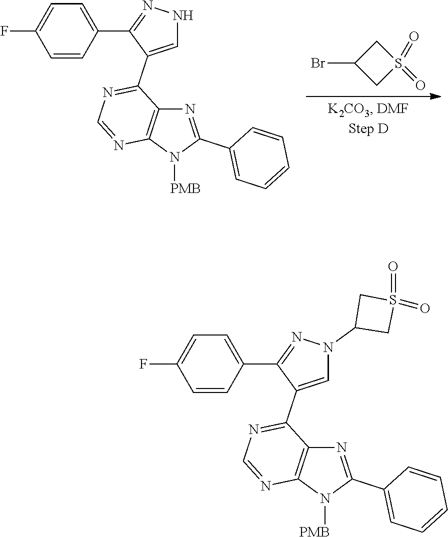

To a stirred mixture of 6-(3-(4-fluorophenyl)-1H-pyrazol-4-yl)-9-(4-methoxybenzyl)-8-phenyl-9H-purine (Intermediate R, 100 mg, 0.210 mmol, 1 equiv) and K₂CO₃ (58.01 mg, 0.420 mmol, 2 equiv) in DMF (3 mL) was added 3-bromo-1,6-thietane-1,1-dione (46.60 mg, 0.252 mmol, 1.2 equiv) at room temperature under nitrogen atmosphere. The resulting mixture was stirred for 16 h at room temperature under nitrogen atmosphere then quenched by the addition of water (10 mL). The resulting mixture was extracted with EtOAc (3×15 mL). The combined organic layers were washed with brine (3×15 mL), dried over anhydrous Na₂SO₄. After filtration, the filtrate was concentrated under reduced pressure. The residue was purified by silica gel column chromatography, eluted with PE:EA (1:4) to afford the title compound (82 mg, 67%) as an off-white solid. m/z (ESI, +ve ion)=581.15 [M+H]⁺.

### Step 2. 3-[3-(4-Fluorophenyl)-4-(8-phenyl-9H-purin-6-yl)-1H-pyrazol-1-yl]-16-thietane-1,1-dione

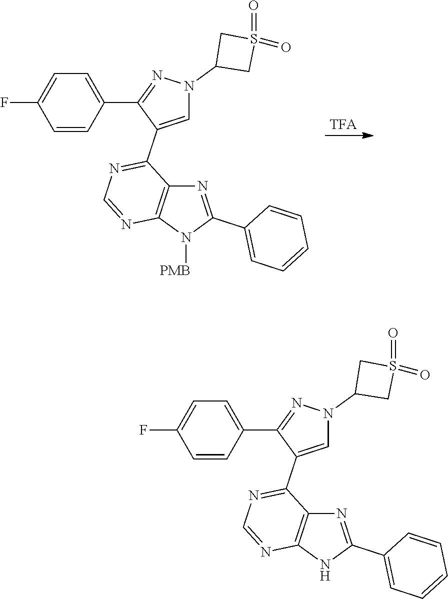

Into a 25 mL round-bottom flask were added 3-[3-(4-fluorophenyl)-4-{9-[(4-methoxyphenyl)methyl]-8-phenylpurin-6-yl}pyrazol-1-yl]-1λ6-thietane-1,1-dione (110 mg, 0.189 mmol, 1 equiv) and TFA (5 mL) at room temperature. The resulting mixture was stirred for 1 h at 70° C. under nitrogen atmosphere then concentrated under reduced pressure. The residue was purified by Prep-HPLC with the following conditions: Column: XBridge Prep Phenyl OBD Column, 19×250 mm, 5 μm; Mobile Phase A: water (0.1% FA), Mobile Phase B: MeOH-HPLC; Flow rate: 25 mL/min; Gradient: 62% B to 72% B in 12 min, 72% B; Wavelength: 254 nm; RT1 (min): 10.6 to afford Example 127 (26.4 mg, 29%) as a white solid. m/z (ESI, +ve ion)=461.15 [M+H]⁺. ¹H NMR (400 MHz, DMSO-d₆) δ 9.20 (s, 1H), 8.67 (s, 1H), 8.34-8.27 (m, 2H), 7.81-7.72 (m, 2H), 7.63-7.57 (m, 3H), 7.27-7.17 (m, 2H), 5.73-5.66 (m, 1H), 4.88 (d, J=7.2 Hz, 4H).

### Example 130. 1-[3-(4-Fluorophenyl)-4-(8-phenyl-9H-purin-6-yl)-1H-pyrazol-1-yl]-2-methylpropan-2-ol

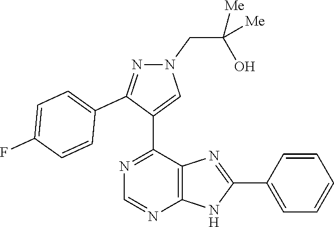 [SMILES: CC(C)(O)Cn1cc(-c2ncnc3[nH]c(-c4ccccc4)nc23)c(-c2ccc(F)cc2)n1]

### Step 1. 1-[3-(4-Fluorophenyl)-4-{9-[(4-methoxyphenyl)methyl]-8-phenylpurin-6-yl}pyrazol-1-yl]-2-methylpropan-2-ol

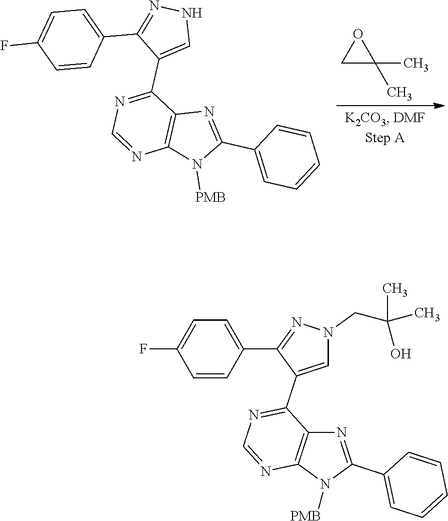

To a stirred mixture of 6-(3-(4-fluorophenyl)-1H-pyrazol-4-yl)-9-(4-methoxybenzyl)-8-phenyl-9H-purine (Intermediate R, 110 mg, 0.231 mmol, 1 equiv) and K₂CO₃ (63.81 mg, 0.462 mmol, 2 equiv) in DMF (3 mL) was added 2,2-dimethyloxirane (19.97 mg, 0.277 mmol, 1.2 equiv) at room temperature under nitrogen atmosphere. The resulting mixture was stirred for 16 h at 60° C. under nitrogen atmosphere then quenched by the addition of sat. NaHCO₃ (aq., 10 mL) at room temperature. The resulting mixture was extracted with EtOAc (3×10 mL). The combined organic layers were washed with brine (3×10 mL), dried over anhydrous Na₂SO₄. After filtration, the filtrate was concentrated under reduced pressure. The residue was purified by silica gel column chromatography, eluted with PE:EA (1:2) to afford the title compound (110 mg, 87%) as a white solid. m/z (ESI, +ve ion)=549.20 [M+H]⁺.

### Step 2. 1-[3-(4-Fluorophenyl)-4-(8-phenyl-9H-purin-6-yl)-1H-pyrazol-1-yl]-2-methylpropan-2-ol

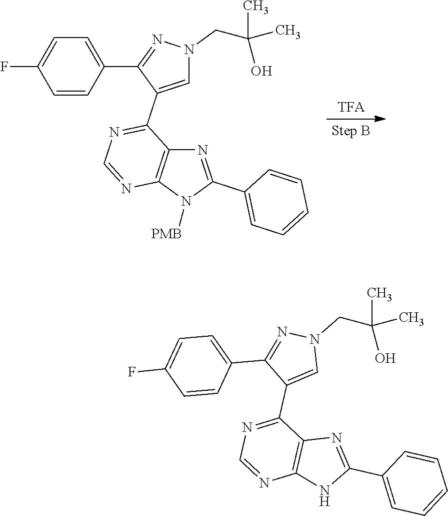

Into a 25 mL round-bottom flask were added 1-[3-(4-fluorophenyl)-4-{9-[(4-methoxyphenyl)methyl]-8-phenylpurin-6-yl}pyrazol-1-yl]-2-methylpropan-2-ol (105 mg, 0.191 mmol, 1 equiv) and TFA (5 mL) at room temperature. The resulting mixture was stirred for 1.5 h at room temperature under nitrogen atmosphere. The resulting mixture was concentrated under reduced pressure. The residue was purified by Prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 25% B to 35% B in 8 min, 35% B; Wavelength: 254 nm; RT1 (min): 7 to afford Example 130 (39.0 mg, 48%) as a white solid. m/z (ESI, +ve ion)=429.20 [M+H]⁺. ¹H NMR (400 MHz, DMSO-d₆) δ 8.99 (s, 1H), 8.63 (s, 1H), 8.31-8.22 (m, 2H), 7.78-7.68 (m, 2H), 7.65-7.52 (m, 3H), 7.24-7.14 (m, 2H), 4.88 (s, 1H), 4.23 (s, 2H), 1.20 (s, 6H).

### Example 134. N-{2-[3-(4-Fluorophenyl)-4-(6-phenylfuro[2,3-d]pyrimidin-4-yl)-1H-pyrazol-1-yl]ethyl}acetamide

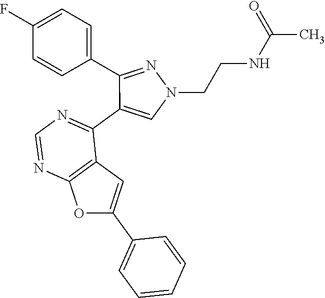 [SMILES: CC(=O)NCCn1cc(-c2ncnc3oc(-c4ccccc4)cc23)c(-c2ccc(F)cc2)n1]

### Step 1. tert-Butyl (2-(3-(4-fluorophenyl)-4-(6-phenylfuro[2,3-d]pyrimidin-4-yl)-1H-pyrazol-1-yl)ethyl)carbamate

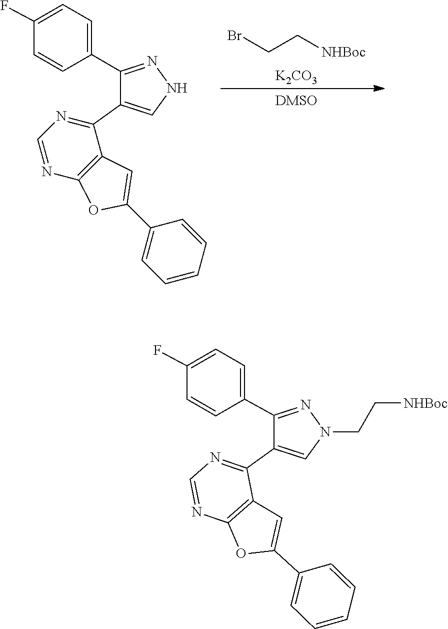

4-[3-(4-Fluorophenyl)-1H-pyrazol-4-yl]-6-phenylfuro[2,3-d]pyrimidine (Intermediate N, 100 mg, 281 μmol) and potassium carbonate (98.9 mg, 702 μmol, 2.5 equiv.) were suspended in DMSO (1.40 mL). After 5 minutes 2-(boc-amino)ethyl bromide (77.8 mg, 337 μmol, 1.2 equiv.) was added. The reaction mixture was stirred at room temperature for 16 h. H₂O (10 mL) and EtOAc (10 mL) were added and the aqueous layer was extracted with additional EtOAc (3×20 mL). The combined organic layers were washed with a saturated aqueous solution of Na₂S₂O₃ (10 mL), H₂O (10 mL), brine (10 mL), dried over Na₂SO₄, filtered, and concentrated to provide the title compound (140 mg, quant.) as an oil. The material was used in the next step without further purification. m/z (ESI, +ve ion)=500.2 [M+H]⁺.

### Step 2. 2-(3-(4-Fluorophenyl)-4-(6-phenylfuro[2,3-d]pyrimidin-4-yl)-1H-pyrazol-1-yl)ethanamine

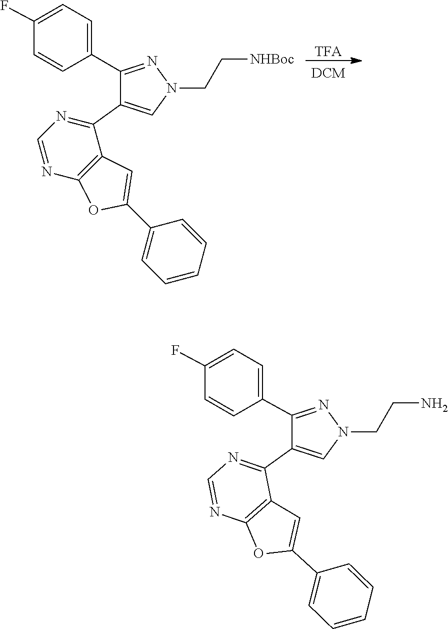 [SMILES: CC(C)(C)OC(=O)NCCn1cc(-c2ncnc3oc(-c4ccccc4)cc23)c(-c2ccc(F)cc2)n1 ; NCCn1cc(-c2ncnc3oc(-c4ccccc4)cc23)c(-c2ccc(F)cc2)n1 ; "TFA"]

tert-butyl (2-(3-(4-fluorophenyl)-4-(6-phenylfuro[2,3-d]pyrimidin-4-yl)-1H-pyrazol-1-yl)ethyl)carbamate (140 mg, 281 μmol) was dissolved in DCM (700 μL) and the solution was cooled to 0° C., then trifluoroacetic acid (694 μL, 8.97 mmol, 32 equiv.) was added slowly. The reaction was stirred at room temperature for 30 minutes. The volatiles were evaporated to provide the title compound (112 mg, quant.) as an oil. The material was used in the next step without any further purification. m/z (ESI, +ve ion)=400.3 [M+H]⁺.

### Step 3. N-{2-[3-(4-Fluorophenyl)-4-(6-phenylfuro[2,3-d]pyrimidin-4-yl)-1H-pyrazol-1-yl]ethyl}acetamide

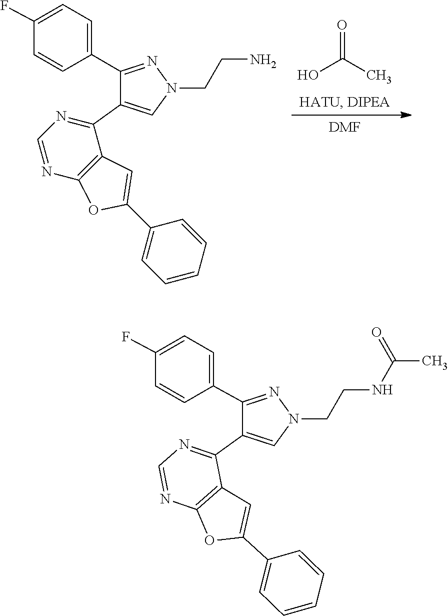

Acetic acid (19.3 μL, 336 μmol, 1.2 equiv.) and N,N-diisopropylethylamine (195 μL, 1.12 mmol, 4.0 equiv.) were added to the solution of 2-(3-(4-fluorophenyl)-4-(6-phenylfuro[2,3-d]pyrimidin-4-yl)-1H-pyrazol-1-yl)ethanamine (112 mg, 280 μmol) in DMF (1.40 mL) at room temperature. HATU (162 mg, 421 μmol, 1.5 equiv.) was added and the reaction mixture was stirred at room temperature for 1 hour. Additional acetic acid (9.7 μL, 168 μmol, 0.6 equiv.) was added and the mixture was stirred at room temperature for 1 hour, then additional acetic acid (9.7 μL, 168 μmol, 0.6 equiv.) was added and the mixture was stirred at room temperature for 16 hours. The mixture was purified by prep HPLC (Buchi Pure C850, Phenomenex Luna C18, 10 μm, 250 mm×21.2 mm, eluent: MeCN in water with 10 mM ammonium formate, 10-100%, a graduate elution) to provide the title compound (19.1 mg, 15%) as a solid. ¹H NMR (500 MHz, DMSO-d₆) δ 8.77 (s, 1H), 8.63 (s, 1H), 8.12 (t, J=5.6 Hz, 1H), 7.92 (d, J=7.4 Hz, 2H), 7.63 (dd, J=8.8, 5.6 Hz, 2H), 7.57 (t, J=7.6 Hz, 2H), 7.51 (d, J=7.2 Hz, 1H), 7.48 (s, 1H), 7.19 (t, J=8.9 Hz, 2H), 4.33 (t, J=6.0 Hz, 2H), 3.60 (dd, J=11.8, 5.9 Hz, 2H), 1.84 (s, 3H). m/z (ESI, +ve ion)=442.7 [M+H]⁺.

### Example 135. 4-[1-(2-Fluoroethyl)-3-(4-fluorophenyl)-1H-pyrazol-4-yl]-6-phenylfuro[2,3-d]pyrimidine

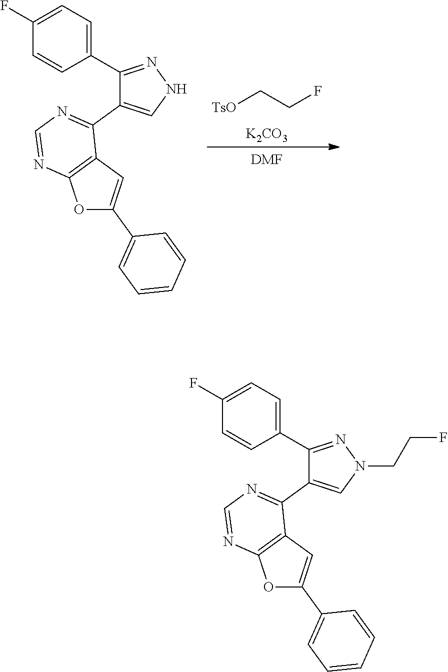

Potassium carbonate (79.1 mg, 561 μmol) was added to a mixture of 4-[3-(4-fluorophenyl)-1H-pyrazol-4-yl]-6-phenylfuro[2,3-d]pyrimidine (Intermediate N, 100 mg, 281 μmol, 2.0 equiv.) and 2-fluoroethyl 4-methylbenzenesulfonate (52.4 μL, 300 μmol, 1.1 equiv.) in DMF (2.80 mL). The mixture was stirred for 15 hours at room temperature, 15 h at 70° C. then diluted with water (20 mL) and extracted with EtOAc (3×10 mL). The combined organic layers were washed with water, brine, then dried (Na₂SO₄), filtered and concentrated. The residue was purified by three consecutive column chromatography (EtOAc in hexanes, 0-50%, a graduate elution) to provide the title compound (35.0 mg, 31%) as a solid. ¹H NMR (500 MHz, DMSO-d₆) δ 8.78 (s, 1H), 8.68 (s, 1H), 7.93-7.88 (m, 2H), 7.64-7.58 (m, 2H), 7.58-7.53 (m, 2H), 7.52-7.46 (m, 1H), 7.35 (s, 1H), 7.20 (t, J=8.9 Hz, 2H), 4.94 (dt, J=47.1, 4.8 Hz, 2H), 4.62 (dt, J=27.4, 4.7 Hz, 2H). m/z (ESI, +ve ion)=403.5 [M+H]⁺.

### Example 136. (R)-4-{3-(4-Fluorophenyl)-1-[(3R)-oxan-3-yl]-1H-pyrazol-4-yl}-6-phenylfuro[2,3-d]pyrimidine

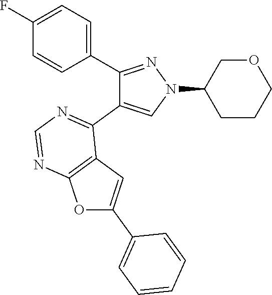 [SMILES: Fc1ccc(-c2nn([C@@H]3CCCOC3)cc2-c2ncnc3oc(-c4ccccc4)cc23)cc1]

### Example 137. (S)-4-{3-(4-Fluorophenyl)-1-[(3S)-oxan-3-yl]-1H-pyrazol-4-yl}-6-phenylfuro[2,3-d]pyrimidine

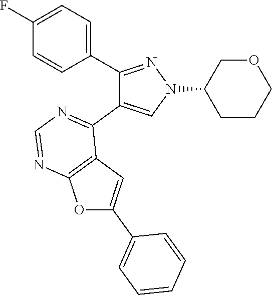 [SMILES: Fc1ccc(-c2nn([C@H]3CCCOC3)cc2-c2ncnc3oc(-c4ccccc4)cc23)cc1]

### Step A. 4-(3-(4-Fluorophenyl)-1-(tetrahydro-2H-pyran-3-yl)-1H-pyrazol-4-yl)-6-phenylfuro[2,3-d]pyrimidine

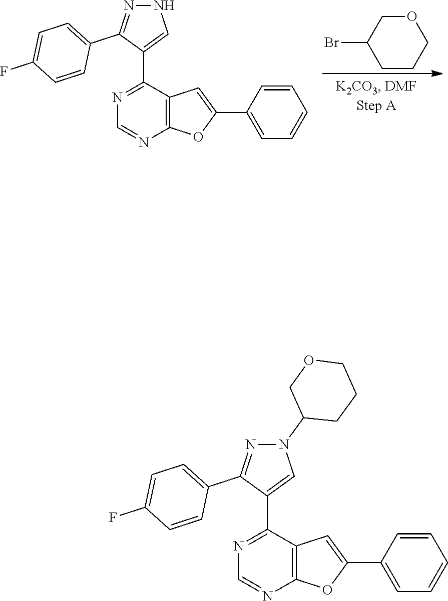 [SMILES: Fc1ccc(-c2n[nH]cc2-c2ncnc3oc(-c4ccccc4)cc23)cc1 ; BrC1CCCOC1 ; Fc1ccc(-c2nn(C3CCCOC3)cc2-c2ncnc3oc(-c4ccccc4)cc23)cc1]

To a stirred solution of 4-[3-(4-Fluorophenyl)-1H-pyrazol-4-yl]-6-phenylfuro[2,3-d]pyrimidine (Intermediate N, 200 mg, 0.561 mmol, 1 equiv) in DMF (1.1 mL) was added K₂CO₃ (155.13 mg, 1.122 mmol, 2 equiv) at room temperature under nitrogen atmosphere. The resulting mixture was stirred for 10 min at room temperature under nitrogen atmosphere. To the above mixture was added 3-bromooxane (111.14 mg, 0.673 mmol, 1.2 equiv) at room temperature. The resulting mixture was stirred for additional 16 h at 100° C. then cooled down to room temperature. The reaction was quenched by the addition of water (10 mL) at 0° C. The resulting mixture was extracted with EtOAc (3×30 mL). The combined organic layers were washed with brine (2×20 mL), dried over anhydrous Na₂SO₄. After filtration, the filtrate was concentrated under reduced pressure. The crude product was purified by prep-HPLC with the following conditions: Column: Xselect CSH C18 OBD Column 30×150 mm 5 μm, Mobile Phase A: water (0.1% FA), Mobile Phase B: ACN; Flow rate: 60 mL/min; Gradient: 63% B to 63% B in 12 min, 63% B; Wavelength: 220 nm; RT1 (min): 8.05, 9.25 (min) to afford Example 122 (4 mg, 1.6%) as a white solid. The racemic Example 122 (100 mg) was separated by Chiral-HPLC with the following conditions: Column: CHIRAL ART Cellulose-SB, 2×25 cm, 5 m; Mobile Phase A: Hex (0.5% 2M NH₃-MeOH)-HPLC, Mobile Phase B: EtOH:DCM=1:1-HPLC; Flow rate: 20 mL/min; Gradient: 20% B to 20% B in 13 min; Wavelength: 220/254 nm; RT1 (min): 9.83; RT2 (min): 11.94; Sample Solvent: MeOH:DCM=1:1-HPLC; Injection Volume: 0.4 mL to give Example 136 (first peak, 27.3 mg, 27%) (absolute stereochemistry arbitrarily assigned) as a white solid and Example 137 (second peak, 26.6 mg, 27%) (absolute stereochemistry arbitrarily assigned) as a white solid. Example 136. m/z (ESI, +ve ion)=441.20 [M+H]⁺. ¹H NMR (400 MHz, Chloroform-d) δ 9.00 (s, 1H), 8.56 (s, 1H), 7.63-7.61 (m, 2H), 7.57-7.53 (m, 2H), 7.48-7.44 (m, 3H), 7.13 (t, J=8.8 Hz, 2H), 6.02 (s, 1H), 4.50-4.47 (m, 1H), 4.29-4.25 (m, 1H), 3.97-3.90 (m, 2H), 3.72-3.61 (m, 1H), 2.38-2.30 (m, 2H), 1.94-1.82 (m, 2H). Example 137. m/z (ESI, +ve ion)=441.20 [M+H]⁺. ¹H NMR (400 MHz, Chloroform-d) δ 9.00 (s, 1H), 8.66 (s, 1H), 7.62-7.60 (m, 2H), 7.56-7.53 (m, 2H), 7.49-7.45 (m, 3H), 7.13 (t, J=8.8 Hz, 2H), 5.99 (s, 1H), 4.51-4.49 (m, 1H), 4.29-4.26 (m, 1H), 3.97-3.90 (m, 2H), 3.70-3.63 (m, 1H), 2.39-2.31 (m, 2H), 1.95-1.83 (m, 2H).

### Example 138. 1-[4-{4-[3-(4-Fluorophenyl)-1-(2-hydroxy-2-methylpropyl)-1H-pyrazol-4-yl]furo[2,3-d]pyrimidin-6-yl}-3,6-dihydropyridin-1 (2H)-yl]ethan-1-one

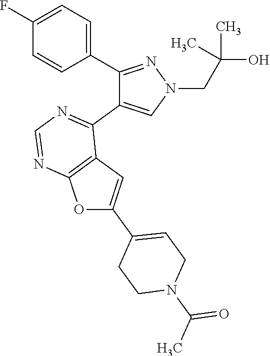 [SMILES: CC(=O)N1CC=C(c2cc3c(-c4cn(CC(C)(C)O)nc4-c4ccc(F)cc4)ncnc3o2)CC1]

### Step 1. 1-(4-(6-Bromofuro[2,3-d]pyrimidin-4-yl)-3-(4-fluorophenyl)-1H-pyrazol-1-yl)-2-methylpropan-2-ol

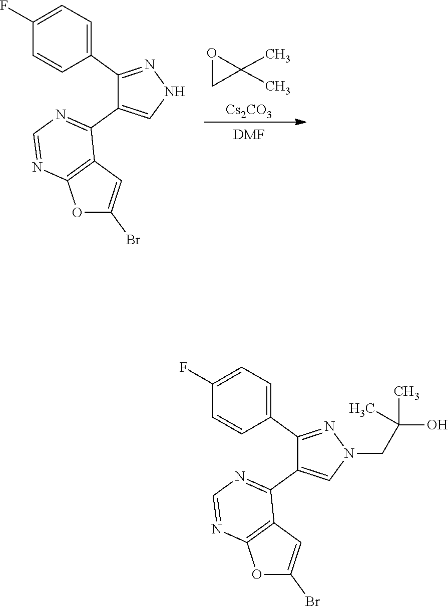

6-Bromo-4-(3-(4-fluorophenyl)-1H-pyrazol-4-yl)furo[2,3-d]pyrimidine (Intermediate Q, 100 mg, 278 μmol) and cesium carbonate (229 mg, 696 μmol, 2.5 equiv.) were suspended in DMF (2.78 mL). The mixture was stirred at room temperature for 30 minutes, then 1,2-epoxy-2-methylpropane (76.5 μL, 835 μmol, 3.0 equiv.) was added. The mixture was heated to 80° C. for 16 h. The mixture was cooled to room temperature and diluted with H₂O (20 mL) and EtOAc (20 mL). The aqueous layer was extracted with additional EtOAc (3×15 mL) and the combined organic layers were washed with H₂O (3×15 mL), brine (10 mL), dried over Na₂SO₄, filtered and concentrated. The residue was purified by column chromatography (EtOAc in hexanes, a gradient of 10-100%) to provide the title compound (as a mixture of N-1 and N-2 alkylated regioisomers) (31.3 mg, 26%) as a solid. ¹H NMR (500 MHz, DMSO-d₆) δ 8.80 and 8.76 (s, 1H), 8.45 and 8.44 (s, 1H), 7.60-7.53 (m, 2H), 7.22-7.14 (m, 2H), 7.02 and 6.92 (s, 1H), 4.82 (s, 1H), 4.16 (s, 2H), 1.17 (s, 6H) (reported as a 5/1 mixture of N-1 and N-2 alkylated regioisomers). m/z (ESI, +ve ion)=431.0, 433.0 [M+H]⁺.

### Step 2. 1-(4-(4-(3-(4-Fluorophenyl)-1-(2-hydroxy-2-methylpropyl)-1H-pyrazol-4-yl)furo[2,3-d]pyrimidin-6-yl)-5,6-dihydropyridin-1 (2H)-yl)ethanone

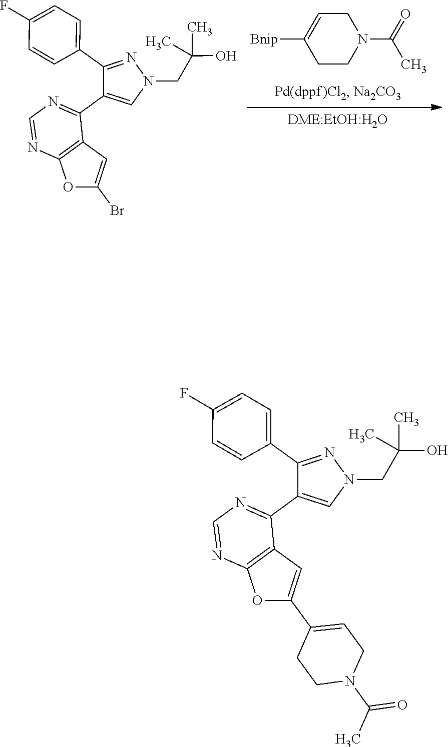

1,1′-Bis(diphenylphosphino)ferrocene dichloropalladium (II) (3.32 mg, 4.31 μmol, 0.1 equiv.) was added to a degassed mixture of 1-(4-(6-bromofuro[2,3-d]pyrimidin-4-yl)-3-(4-fluorophenyl)-1H-pyrazol-1-yl)-2-methylpropan-2-ol (18.6 mg, 43.1 μmol), 1-(4-(4,4,5,5-tetramethyl-1,3,2-dioxaborolan-2-yl)-5,6-dihydropyridin-1 (2H)-yl)ethanone (11.9 mg, 47.4 μmol, 1.1 equiv.), and sodium carbonate (13.9 mg, 129 μmol, 3.0 equiv.) dissolved in a mixture of DME (372 μL), EtOH (186 μL), and H₂O (93.0 μL). The mixture was further degassed with nitrogen for 3 minutes then heated at 115° C. for 1.5 h in a microwave reactor. After cooling to room temperature, the mixture was diluted with EtOAc, filtered, and concentrated under reduced pressure. The residue was purified by reverse phase chromatography (C18 column, MeCN in water with 10 mM ammonium formate, 0-35%, a gradient elution), followed by column chromatography (MeOH in DCM, 0-5%, a gradient elution) to provide the title compound (11.4 mg, 56%) as a solid. ¹H NMR (400 MHz, DMSO-d₆) δ 8.71 (s, 1H), 8.40 (d, J=2.5 Hz, 1H), 7.55-7.48 (m, 2H), 7.15 (app t, J=8.9 Hz, 2H), 6.57-6.51 (m, 2H), 4.81 (s, 1H), 4.21 (app s, 1H), 4.13 (app s, 1H), 4.13 (s, 2H), 3.64-3.56 (m, 2H), 2.38 (app s, 1H), 2.28 (app s, 1H), 2.04 and 2.01 (s, 3H), 1.14 (s, 6H). m/z (ESI, +ve ion)=476.4 [M+H]⁺.

### Example 140. 3-[4-{6-[(1-Acetylazetidin-3-yl)ethynyl]furo[2,3-d]pyrimidin-4-yl}-3-(4-fluorophenyl)-1H-pyrazol-1-yl]-16-thietane-1,1-dione

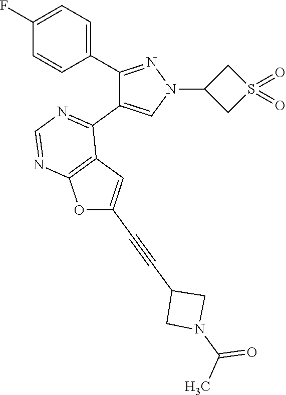 [SMILES: CC(=O)N1CC(C#Cc2cc3c(-c4cn(C5CS(=O)(=O)C5)nc4-c4ccc(F)cc4)ncnc3o2)C1]

### Step 1. 3-(4-(6-Bromofuro[2,3-d]pyrimidin-4-yl)-3-(4-fluorophenyl)-1H-pyrazol-1-yl)thietane 1,1-dione

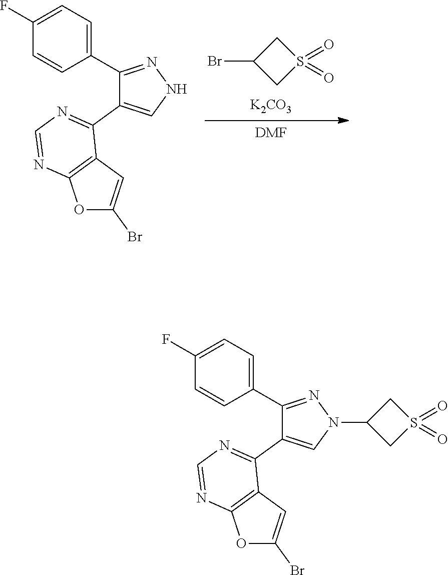 [SMILES: Fc1ccc(-c2n[nH]cc2-c2ncnc3oc(Br)cc23)cc1 ; O=S1(=O)CC(Br)C1 ; O=S1(=O)CC(n2cc(-c3ncnc4oc(Br)cc34)c(-c3ccc(F)cc3)n2)C1]

6-Bromo-4-(3-(4-fluorophenyl)-1H-pyrazol-4-yl)furo[2,3-d]pyrimidine (Intermediate Q, 500 mg, 1.39 mmol) and potassium carbonate (393 mg, 2.78 mmol, 2.0 equiv.) were dissolved in DMF (11.2 mL). After stirring for 5 min 3-bromothietane 1,1-dioxide (407 mg, 2.09 mmol, 1.5 equiv.) was added. The mixture was further stirred at room temperature for 16 h. H₂O (15 mL), brine and EtOAc (15 mL) were added. The aqueous layer was extracted with additional EtOAc (3×30 mL). The combined organic layers were washed with H₂O (3×20 mL), brine (20 mL), dried over Na₂SO₄, filtered and concentrated. The crude product was purified by column chromatography (EtOAc in hexanes, 10-100%, a gradient elution) to provide the title compound (488 mg, 76%) as a solid. m/z (ESI, +ve ion)=463.0, 465.0 [M+H]⁺.

### Step 2. tert-Butyl 3-({4-[1-(1,1-dioxo-16-thietan-3-yl)-3-(4-fluorophenyl)-1H-pyrazol-4-yl]furo[2,3-d]pyrimidin-6-yl}ethynyl)azetidine-1-carboxylate

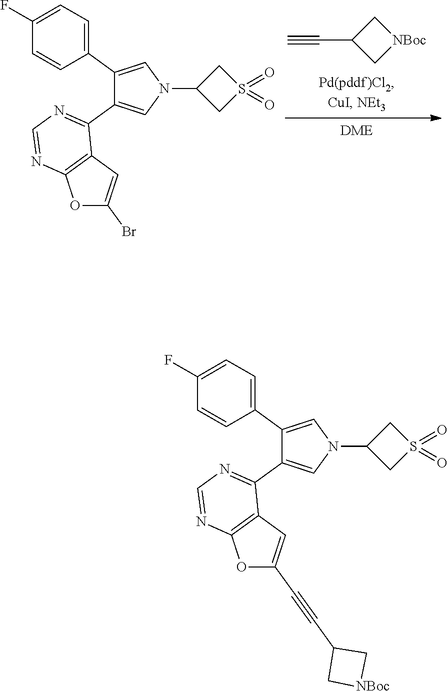

3-(4-(6-Bromofuro[2,3-d]pyrimidin-4-yl)-3-(4-fluorophenyl)-1H-pyrazol-1-yl)thietane 1,1-dione (150 mg, 324 μmol), copper(I) iodide (6.17 mg, 32.4 μmol, 0.1 equiv.), and 1,1′-bis(diphenylphosphino)ferrocene dichloropalladium (II) (24.9 mg, 32.4 μmol, 0.1 equiv.) were dissolved in DME (2.91 mL) under nitrogen. The mixture was degassed with nitrogen for 5 minutes before adding triethylamine (137 μL, 971 μmol, 3.0 equiv.) and tert-butyl 3-ethynylazetidine-1-carboxylate (86.4 μL, 486 μmol, 1.5 equiv.). The reaction mixture was degassed for an additional 2 minutes, then heated at 80° C. for 16 h. The reaction mixture was cooled to room temperature, diluted with EtOAc then filtered. water (15 mL) was added and the aqueous layer was extracted with additional EtOAc (3×15 mL). The combined organic layers were washed with H₂O (15 mL), brine (15 mL), dried over Na₂SO₄, filtered and concentrated. The residue was purified by column chromatography (EtOAc in hexanes, 10-100%, a gradient elution) followed by reverse phase chromatography (C18 column, MeCN in water with 10 mM ammonium formate, 10-100%, a gradient elution) to provide the title compound (130 mg, 71%) as a solid. ¹H NMR (500 MHz, DMSO-d₆) δ 8.83 (s, 1H), 8.75 (s, 1H), 7.65-7.56 (m, 2H), 7.34 (s, 1H), 7.24-7.15 (m, 2H), 5.47 (tt, J=8.8, 5.5 Hz, 1H), 4.95-4.83 (m, 4H), 4.21 (t, J=8.1 Hz, 2H), 3.92-3.87 (m, 2H), 3.87-3.80 (m, 1H), 1.40 (s, 9H). m/z (ESI, +ve ion)=564.4 [M+H]⁺.

### Step 3. 3-[4-{6-[(Azetidin-3-yl)ethynyl]furo[2,3-d]pyrimidin-4-yl}-3-(4-fluorophenyl)-1H-pyrazol-1-yl]-1λ6-thietane-1,1-dione

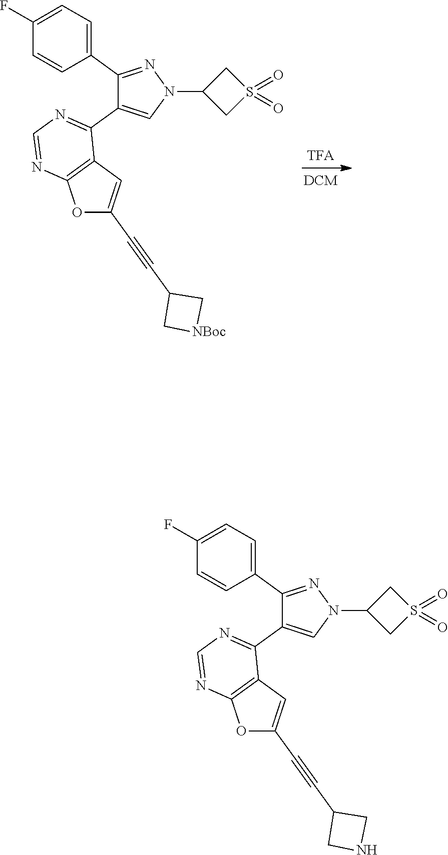 [SMILES: CC(C)(C)OC(=O)N1CC(C#Cc2cc3c(-c4cn(C5CS(=O)(=O)C5)nc4-c4ccc(F)cc4)ncnc3o2)C1 ; O=S1(=O)CC(n2cc(-c3ncnc4oc(C#CC5CNC5)cc34)c(-c3ccc(F)cc3)n2)C1]

tert-butyl 3-({4-[1-(1,1-dioxo-1λ6-thietan-3-yl)-3-(4-fluorophenyl)-1H-pyrazol-4-yl]furo[2,3-d]pyrimidin-6-yl}ethynyl)azetidine-1-carboxylate (115 mg, 204 μmol) was dissolved in DCM (2.40 mL) and the mixture was cooled to 0° C. Trifluoroacetic acid (158 μL, 2.04 mmol, 10 equiv.) was added and the mixture was stirred at 0° C. for 30 minutes. The mixture was warmed to room temperature and stirred for an additional 16 h. The volatiles were removed in vacuo and the residue was purified by reverse phase chromatography (C18 column, MeCN in water with 10 mM ammonium formate, 5-35%, a gradient elution) to provide the title compound formate salt (97.8 mg, 99%) as a solid. ¹H NMR (500 MHz, DMSO-d₆) δ 8.83 (s, 1H), 8.75 (s, 1H), 8.21 (s, 1H), 7.67-7.55 (m, 2H), 7.29 (s, 1H), 7.25-7.14 (m, 2H), 5.47 (tt, J=8.7, 5.5 Hz, 1H), 4.98-4.82 (m, 4H), 3.90-3.82 (m, 1H), 3.79 (app t, J=7.7 Hz, 2H), 3.69 (app t, J=7.3 Hz, 2H). m/z (ESI, +ve ion)=464.3 [M+H]⁺.

### Step 4. 3-[4-{6-[(1-Acetylazetidin-3-yl)ethynyl]furo[2,3-d]pyrimidin-4-yl}-3-(4-fluorophenyl)-1H-pyrazol-1-yl]-16-thietane-1,1-dione

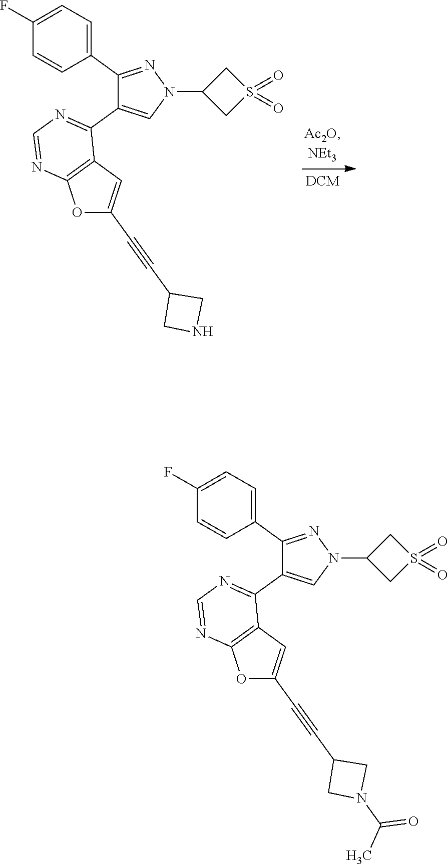 [SMILES: O=S1(=O)CC(n2cc(-c3ncnc4oc(C#CC5CNC5)cc34)c(-c3ccc(F)cc3)n2)C1 ; CC(=O)N1CC(C#Cc2cc3c(-c4cn(C5CS(=O)(=O)C5)nc4-c4ccc(F)cc4)ncnc3o2)C1]

Triethylamine (30.4 μL, 216 μmol, 2.0 equiv.) was added to a mixture of 3-[4-{6-[(azetidin-3-yl)ethynyl]furo[2,3-d]pyrimidin-4-yl}-3-(4-fluorophenyl)-1H-pyrazol-1-yl]-1λ6-thietane-1,1-dione (50.0 mg, 108 μmol, as a formate salt) in DCM (2.5 mL). Acetic anhydride (15.5 μL, 162 μmol, 1.5 equiv.) was added to the mixture and it was stirred at room temperature for 2 h then concentrated under reduced pressure. The crude was purified by reverse phase chromatography (C18 column, MeCN in water with 10 mM ammonium formate, 10-40%, a gradient elution) to provide the title compound (42.3 mg, 78%) as a solid. ¹H NMR (400 MHz, DMSO-d₆) δ 8.83 (s, 1H), 8.75 (s, 1H), 7.66-7.56 (m, 2H), 7.35 (s, 1H), 7.25-7.16 (m, 2H), 5.47 (tt, J=8.6, 5.5 Hz, 1H), 4.96-4.82 (m, 4H), 4.47 (app t, J=8.2 Hz, 1H), 4.24-4.16 (m, 2H), 3.90-3.83 (m, 2H), 1.78 (s, 3H). m/z (ESI, +ve ion)=506.3 [M+H]⁺.

### Example 141. N-{3-[3-(4-Fluorophenyl)-4-(6-phenylfuro[2,3-d]pyrimidin-4-yl)-1H-pyrazol-1-yl]propyl}acetamide

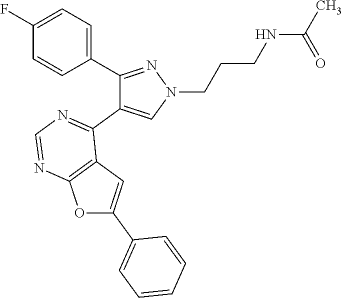 [SMILES: CC(=O)NCCCn1cc(-c2ncnc3oc(-c4ccccc4)cc23)c(-c2ccc(F)cc2)n1]

### Step 1. tert-Butyl (3-(3-(4-fluorophenyl)-4-(6-phenylfuro[2,3-d]pyrimidin-4-yl)-1H-pyrazol-1-yl)propyl)carbamate

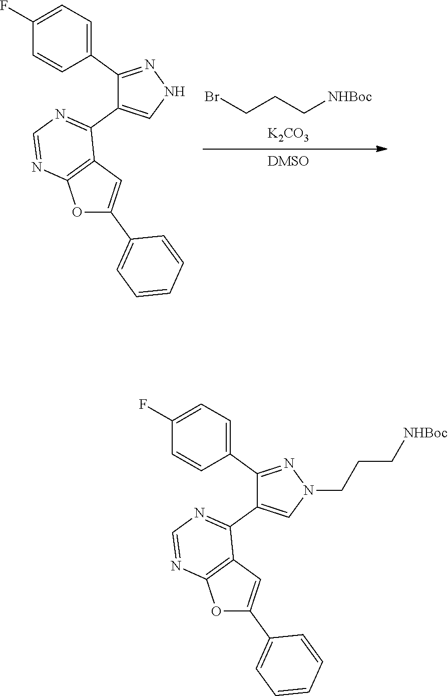 [SMILES: Fc1ccc(-c2n[nH]cc2-c2ncnc3oc(-c4ccccc4)cc23)cc1 ; CC(C)(C)OC(=O)NCCCBr ; CC(C)(C)OC(=O)NCCCn1cc(-c2ncnc3oc(-c4ccccc4)cc23)c(-c2ccc(F)cc2)n1]

4-[3-(4-Fluorophenyl)-1H-pyrazol-4-yl]-6-phenylfuro[2,3-d]pyrimidine (Intermediate N, 100 mg, 281 μmol) was added to a mixture of potassium carbonate (98.9 mg, 702 μmol, 2.5 equiv.) in DMSO (1.40 mL). After 5 minutes 3-(boc-amino)propyl bromide (81.0 mg, 337 μmol, 1.2 equiv.) was added. The reaction mixture was stirred at room temperature for 72 h. H₂O (10 mL) and EtOAc (10 mL) were added and the aqueous layer was extracted with additional EtOAc (3×20 mL). The combined organic layers were washed with a saturated aqueous solution of Na₂S₂O₃ (10 mL), H₂O (10 mL), brine (10 mL), dried over Na₂SO₄, filtered, and concentrated to provide the title compound (144 mg, quant.) as an oil. The material was used in the next step without further purification. m/z (ESI, +ve ion)=514.5 [M+H]⁺.

### Step 2. 3-(3-(4-Fluorophenyl)-4-(6-phenylfuro[2,3-d]pyrimidin-4-yl)-1H-pyrazol-1-yl)propan-1-amine

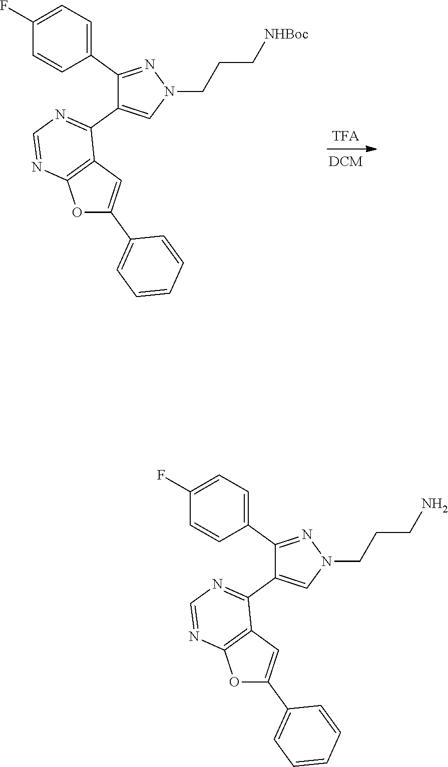 [SMILES: CC(C)(C)OC(=O)NCCCn1cc(-c2ncnc3oc(-c4ccccc4)cc23)c(-c2ccc(F)cc2)n1 ; NCCCn1cc(-c2ncnc3oc(-c4ccccc4)cc23)c(-c2ccc(F)cc2)n1]

Trifluoroacetic acid (694 μL, 8.97 mmol, 32 equiv.) was slowly added to a mixture of tert-butyl (3-(3-(4-fluorophenyl)-4-(6-phenylfuro[2,3-d]pyrimidin-4-yl)-1H-pyrazol-1-yl)propyl)carbamate (144 mg, 280 μmol) in DCM (701 μL) at 0° C. The mixture was stirred at room temperature for 30 minutes the concentrated to provide the title compound (116 mg, quant., as the TFA salt) as an oil. The material was used in the next step without any further purification. m/z (ESI, +ve ion)=414.4 [M+H]⁺.

### Step 3. N-{3-[3-(4-Fluorophenyl)-4-(6-phenylfuro[2,3-d]pyrimidin-4-yl)-1H-pyrazol-1-yl]propyl}acetamide

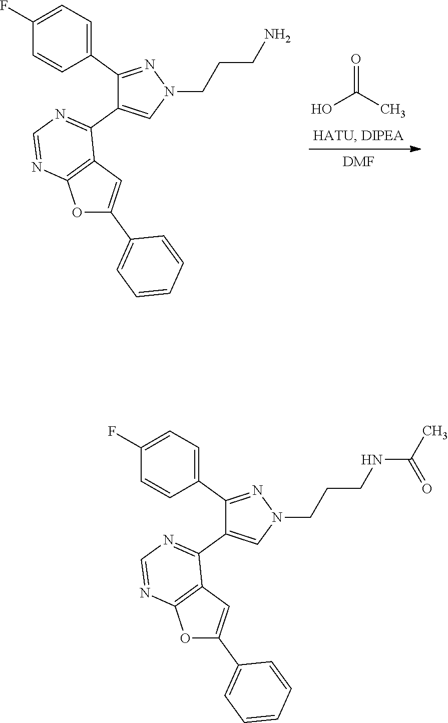

Acetic acid (64.9 μL, 1.12 mmol, 4.0 equiv.) and N,N-diisopropylethylamine (195 μL, 1.12 mmol, 4.0 equiv.) were added to the solution of 3-(3-(4-fluorophenyl)-4-(6-phenylfuro[2,3-d]pyrimidin-4-yl)-1H-pyrazol-1-yl)propan-1-amine (116 mg, 280 μmol) in DMF (1.40 mL) at room temperature. HATU (162 mg, 421 μmol, 1.5 equiv.) was added and the reaction mixture was stirred at room temperature for 16 hours. Additional acetic acid (16.1 μL, 280 μmol, 1.0 equiv.) was added and the mixture was stirred at room temperature for 24 hours. The mixture was purified by prep HPLC (Buchi Pure C850, Phenomenex Luna C18, 10 μm, 250 mm×21.2 mm, eluent: MeCN in water with 10 mM ammonium formate, 10-100%, a gradient elution) to provide the title compound (35.8 mg, 28%) as a solid. ¹H NMR (400 MHz, DMSO-d₆) δ 8.76 (s, 1H), 8.63 (s, 1H), 8.01-7.97 (m, 1H), 7.97-7.93 (m, 2H), 7.64-7.54 (m, 5H), 7.53-7.46 (m, 1H), 7.22-7.15 (m, 2H), 4.28 (t, J=6.8 Hz, 2H), 3.14 (q, J=6.6 Hz, 2H), 2.09-2.01 (m, 2H), 1.85 (s, 3H). m/z (ESI, +ve ion)=456.4 [M+H]⁺.

### Example 143. 2-[3-(4-Fluorophenyl)-4-(6-phenylfuro[2,3-d]pyrimidin-4-yl)-1H-pyrazol-1-yl]-N-methylacetamide

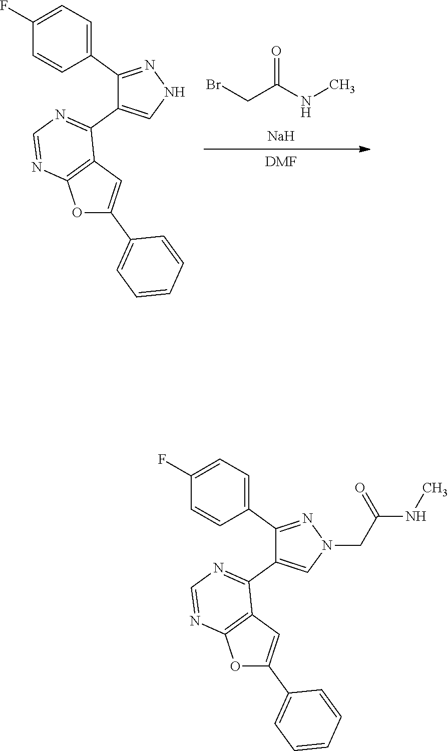

4-[3-(4-Fluorophenyl)-1H-pyrazol-4-yl]-6-phenylfuro[2,3-d]pyrimidine (Intermediate N, 100 mg, 281 μmol) was dissolved in DMF (935 μL) then sodium hydride (8.08 mg, 337 μmol, 1.2 equiv., 60% in dispersion in mineral oil) was added followed by 2-bromo-N-methylacetamide (47.9 mg, 309 μmol, 1.1 equiv.) and the reaction mixture was stirred at room temperature 16 hours. The reaction mixture was purified by prep HPLC (Buchi Pure C850, Phenomenex Luna C18, 10 μm, 250 mm×21.2 mm, eluent: MeCN in water with 10 mM ammonium formate, a gradient of 10-100%) to provide the title compound (40.8 mg, 33%) as a solid. ¹H NMR (400 MHz, DMSO-d₆) δ 8.79 (s, 1H), 8.65 (s, 1H), 8.19 (q, J=4.3 Hz, 1H), 7.92-7.87 (m, 2H), 7.63-7.53 (m, 4H), 7.52-7.46 (m, 1H), 7.33 (s, 1H), 7.23-7.16 (m, 2H), 4.95 (s, 2H), 2.68 (d, J=4.6 Hz, 3H). m/z (ESI, +ve ion)=428.3 [M+H]⁺.

### Example 144. 4-{3-(4-Fluorophenyl)-1-[2-(trifluoromethoxy)ethyl]-1H-pyrazol-4-yl}-6-phenylfuro[2,3-d]pyrimidine

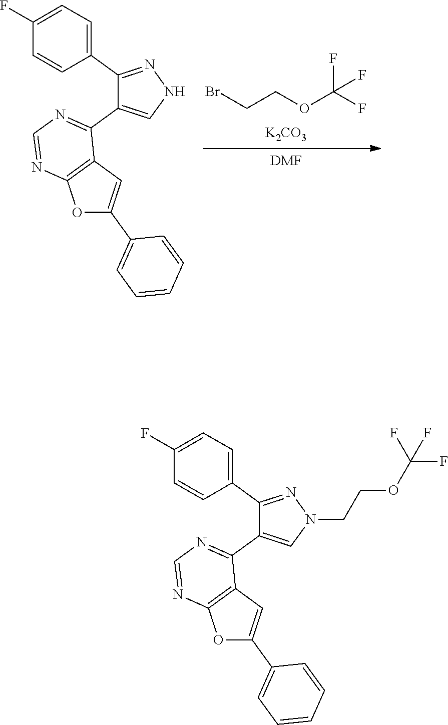 [SMILES: Fc1ccc(-c2n[nH]cc2-c2ncnc3oc(-c4ccccc4)cc23)cc1 ; FC(F)(F)OCCBr ; Fc1ccc(-c2nn(CCOC(F)(F)F)cc2-c2ncnc3oc(-c4ccccc4)cc23)cc1]

Potassium carbonate (79.1 mg, 561 μmol, 2.0 equiv.) was added to a mixture of 4-[3-(4-fluorophenyl)-1H-pyrazol-4-yl]-6-phenylfuro[2,3-d]pyrimidine (Intermediate N, 100 mg, 281 μmol) and 1-bromo-2-(trifluoromethoxy)ethane (35.7 μL, 300 μmol, 1.1 equiv.) in DMF (2.80 mL). The mixture was stirred for 15 h at room temperature, then at 15 h at 60° C. The mixture was cooled to room temperature then diluted with water (20 mL) and extracted with EtOAc (3×10 mL). The combined organic layers were washed with water, brine, dried (Na₂SO₄), filtered then concentrated under reduced pressure. The residue was purified by column chromatography (EtOAc in hexanes, 0-50%, a gradient elution), followed by prep TLC (1:1 mixture of Et₂O:hexanes) to provide the title compound (22.0 mg, 17%) as a solid. ¹H NMR (400 MHz, DMSO-d₆) δ 8.79 (s, 1H), 8.71 (s, 1H), 7.94-7.86 (m, 2H), 7.64-7.53 (m, 4H), 7.52-7.46 (m, 1H), 7.34 (s, 1H), 7.20 (t, J=8.9 Hz, 2H), 4.63 (s, 4H). m/z (ESI, +ve ion)=469.8 [M+H]⁺.

### Example 147. 4-[3-(4-Fluorophenyl)-4-(6-phenylfuro[2,3-d]pyrimidin-4-yl)-1H-pyrazol-1-yl]butanamide

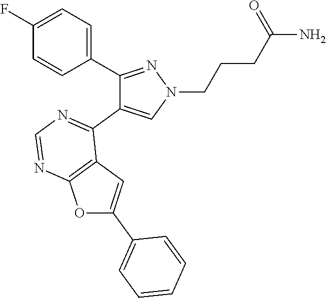 [SMILES: NC(=O)CCCn1cc(-c2ncnc3oc(-c4ccccc4)cc23)c(-c2ccc(F)cc2)n1]

### Step 1. Ethyl 4-(3-(4-fluorophenyl)-4-(6-phenylfuro[2,3-d]pyrimidin-4-yl)-1H-pyrazol-1-yl)butanoate

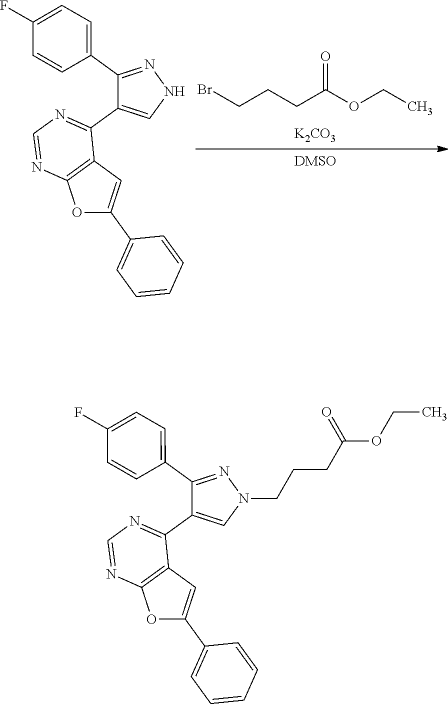

4-[3-(4-Fluorophenyl)-1H-pyrazol-4-yl]-6-phenylfuro[2,3-d]pyrimidine (Intermediate N, 95.0 mg, 267 μmol) and potassium carbonate (94.0 mg, 666 μmol, 2.5 equiv.) were suspended in DMSO (1.33 mL). After 5 minutes, ethyl 4-bromobutyrate (48.2 μL, 320 μmol, 1.2 equiv.) was added. The reaction mixture was stirred at room temperature for 16 hours. H₂O (10 mL) and EtOAc (10 mL) were added and the aqueous layer was extracted with additional EtOAc (3×20 mL). The combined organic layers were washed with water three times, then with brine two times, dried over Na₂SO₄, filtered, and concentrated under reduced pressure to afford the title compound (125 mg, quant) as an oil. m/z (ESI, +ve ion)=472.4 [M+H]⁺.

### Step 2. 4-(3-(4-Fluorophenyl)-4-(6-phenylfuro[2,3-d]pyrimidin-4-yl)-1H-pyrazol-1-yl)butanoic acid

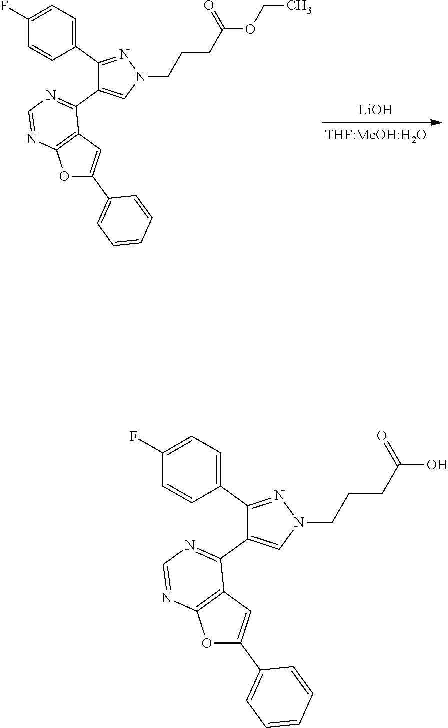 [SMILES: CCOC(=O)CCCn1cc(-c2ncnc3oc(-c4ccccc4)cc23)c(-c2ccc(F)cc2)n1 ; O=C(O)CCCn1cc(-c2ncnc3oc(-c4ccccc4)cc23)c(-c2ccc(F)cc2)n1]

Ethyl 4-(3-(4-fluorophenyl)-4-(6-phenylfuro[2,3-d]pyrimidin-4-yl)-1H-pyrazol-1-yl)butanoate (125 mg, 266 μmol) was dissolved in a mixture of THF (1.9 mL), MeOH (480 μL), and water (960 μL). Lithium hydroxide (33 mg, 1.33 mmol, 5.0 equiv.) was added and the mixture was stirred at room temperature for 2 hours. The mixture was diluted with water (5 mL) and EtOAc (5 mL). The layers were separated, and the aqueous layer was washed with EtOAc (2×5 mL). The aqueous layer was adjusted to pH=4 using 1 M HCl aqueous solution. The aqueous layer was extracted with EtOAc (3×5 mL). The combined organic layers were dried over Na₂SO₄, filtered, and concentrated under reduced pressure to provide the title compound (118 mg, quant) as a solid. m/z (ESI, +ve ion)=443.3 [M+H]⁺.

### Step 3. 4-[3-(4-Fluorophenyl)-4-(6-phenylfuro[2,3-d]pyrimidin-4-yl)-1H-pyrazol-1-yl]butanamide

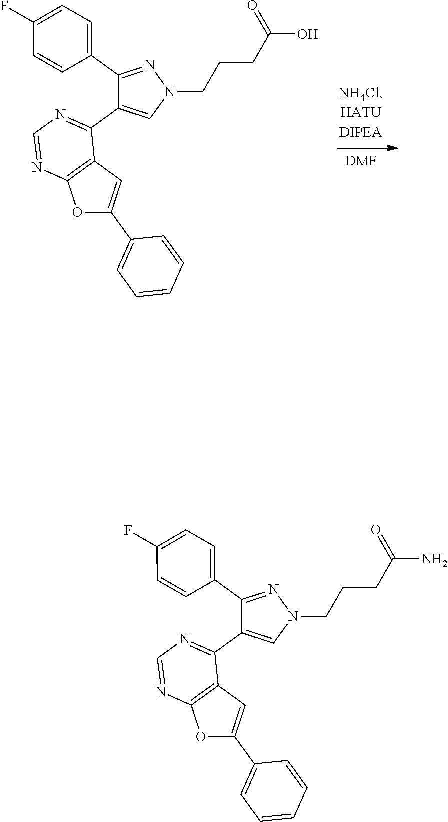 [SMILES: O=C(O)CCCn1cc(-c2ncnc3oc(-c4ccccc4)cc23)c(-c2ccc(F)cc2)n1 ; NC(=O)CCCn1cc(-c2ncnc3oc(-c4ccccc4)cc23)c(-c2ccc(F)cc2)n1]

N,N-Diisopropylethylamine (116 μL, 667 μmol, 2.5 equiv.) and ammonium chloride (57.1 mg, 1.07 mmol, 4.0 equiv.) were added to a solution of 4-(3-(4-fluorophenyl)-4-(6-phenylfuro[2,3-d]pyrimidin-4-yl)-1H-pyrazol-1-yl)butanoic acid (118 mg, 267 μmol) in DMF (1.3 mL) at room temperature. HATU (154 mg, 400 μmol, 1.5 equiv.) was added and the reaction mixture was stirred at room temperature for 1 hour. The mixture was purified by prep-HPLC (waters CSH C18 OBD Prep Column, 30 mm×75 mm, 5 μm, eluent: MeOH in 10 mM ammonium formate in water, 40-100%, a gradient elution) to afford the title compound (21 mg, 17%) as a solid. ¹H NMR (400 MHz, DMSO-d₆) δ 8.76 (s, 1H), 8.64 (s, 1H), 7.95-7.89 (m, 2H), 7.65-7.52 (m, 4H), 7.52-7.48 (m, 1H), 7.47 (s, 1H), 7.36 (s, 1H), 7.22-7.15 (m, 2H), 6.84 (s, 1H), 4.28 (t, J=6.3 Hz, 2H), 2.19-2.11 (m, 4H). m/z (ESI, +ve ion)=442.3 [M+H]⁺.

### Example 148. 3-[3-(4-Fluorophenyl)-4-(6-phenylfuro[2,3-d]pyrimidin-4-yl)-1H-pyrazol-1-yl]-1-methylpyrrolidin-2-one

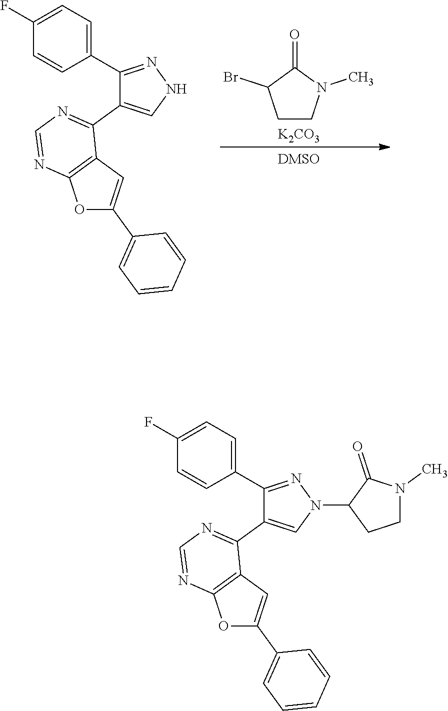

4-[3-(4-Fluorophenyl)-1H-pyrazol-4-yl]-6-phenylfuro[2,3-d]pyrimidine (Intermediate N, 100 mg, 281 μmol) and potassium carbonate (99 mg, 702 μmol, 2.5 equiv.) were suspended in DMF (1.4 mL) and after 5 minutes, 3-bromo-1-methylpyrrolidin-2-one (63 mg, 337 μmol, 1.2 equiv.) was added. The reaction mixture was stirred at 60° C. for 72 hours. The reaction mixture was cooled to room temperature, diluted with water, and extracted with EtOAc three times. The combined organic layers were washed with water (three times), brine, dried over Na₂SO₄, filtered, and concentrated under reduced pressure. The crude material was dissolved in DMSO and purified by prep-HPLC (Buchi Pure C850, Phenomenex Luna C18, 10 μm, 250 mm×21.2 mm, eluent: MeCN with 10 mM ammonium formate in water, a gradient of 55-65%) to afford the title compound (50 mg, 38%) as a solid. ¹H NMR (400 MHz, DMSO-d₆) δ 8.79 (s, 1H), 8.72 (s, 1H), 7.93-7.88 (m, 2H), 7.60-7.53 (m, 4H), 7.53-7.45 (m, 1H), 7.35 (s, 1H), 7.22-7.16 (m, 2H), 5.29 (t, J=9.0 Hz, 1H), 3.57 (td, J=9.2, 3.0 Hz, 1H), 3.53-3.44 (m, 1H), 2.86 (s, 3H), 2.68-2.57 (m, 2H). m/z (ESI, +ve ion)=454.3 [M+H]⁺.

### Example 150. 4-{1-[(1s,3s)-3-fluorocyclobutyl]-3-(4-fluorophenyl)-1H-pyrazol-4-yl}-6-phenylfuro[2,3-d]pyrimidine

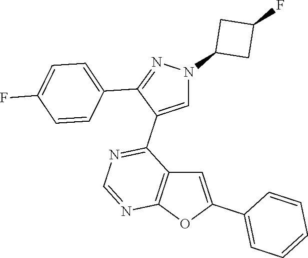 [SMILES: Fc1ccc(-c2nn([C@H]3C[C@@H](F)C3)cc2-c2ncnc3oc(-c4ccccc4)cc23)cc1]

### Step 1. 3-fluorocyclobutyl 4-methylbenzenesulfonate

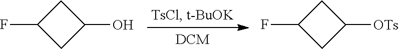 [SMILES: OC1CC(F)C1 ; Cc1ccc(S(=O)(=O)OC2CC(F)C2)cc1]

To a stirred solution of 3-fluorocyclobutan-1-ol (200 mg, 2.220 mmol, 1 equiv.) in DCM (4 mL) was added t-BuOK (373.64 mg, 3.330 mmol, 1.5 equiv.) in portions at 0° C. under nitrogen atmosphere. The resulting mixture was stirred for 30 min at 0° C. under nitrogen atmosphere. To the above mixture was added TsCl (507.83 mg, 2.664 mmol, 1.2 equiv.) in portions at 0° C. The resulting mixture was stirred for additional 1 h at room temperature. The resulting mixture was diluted with water (10 mL) and then extracted with EtOAc (3×15 mL). The combined organic layer was washed with brine (2×30 mL), dried over anhydrous Na₂SO₄. After filtration, the filtrate was concentrated under reduced pressure. The residue was purified by silica gel column chromatography, eluted with PE/EtOAc (9/1) to afford the title compound (480 mg, 89%) as a colorless oil. ¹H-NMR (400 MHz, DMSO-d₆) δ 7.84-7.77 (m, 2H), 7.41-7.34 (m, 2H), 5.34-5.09 (m, 1H), 5.08-5.03 (m, 1H), 2.63-2.56 (m, 2H), 2.56-2.50 (m, 2H), 2.48 (s, 3H).

### Step 2. 4-{1-[(1s,3s)-3-fluorocyclobutyl]-3-(4-fluorophenyl)-1H-pyrazol-4-yl}-6-phenylfuro[2,3-d]pyrimidine

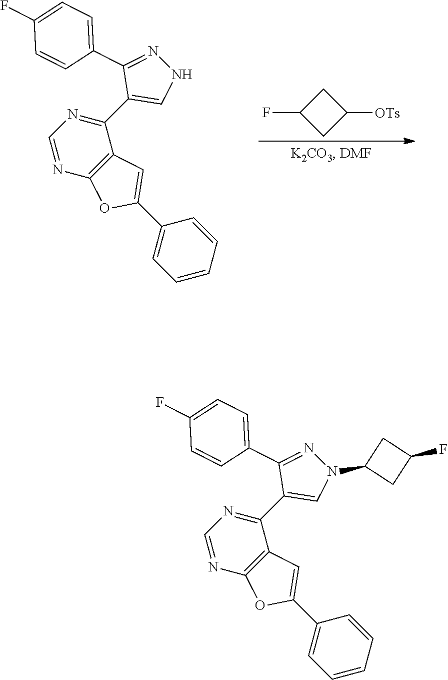 [SMILES: Fc1ccc(-c2n[nH]cc2-c2ncnc3oc(-c4ccccc4)cc23)cc1 ; Cc1ccc(S(=O)(=O)OC2CC(F)C2)cc1 ; Fc1ccc(-c2nn([C@H]3C[C@@H](F)C3)cc2-c2ncnc3oc(-c4ccccc4)cc23)cc1]

To a stirred solution of 4-[3-(4-fluorophenyl)-1H-pyrazol-4-yl]-6-phenylfuro[2,3-d]pyrimidine (Intermediate N, 100 mg, 0.281 mmol, 1 equiv.) and K₂CO₃ (77.56 mg, 0.562 mmol, 2 equiv.) in DMF (1 mL) at room temperature under nitrogen atmosphere was added 3-fluorocyclobutyl 4-methylbenzenesulfonate (82.26 mg, 0.337 mmol, 1.2 equiv.). The resulting mixture was stirred for 3 h at 100° C. under nitrogen atmosphere then quenched by the addition of water (6 mL) at room temperature. The resulting mixture was extracted with EtOAc (3×10 mL). The combined organic layers were washed with brine (2×10 mL), and dried over anhydrous Na₂SO₄. After filtration, the filtrate was concentrated under reduced pressure. The residue was purified by prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 57% B to 67% B in 8 min, 67% B; Wavelength: 254 nm; RT1 (min): 7 to afford Example 150 (72.2 mg, 60%) as a white solid. m/z (ESI, +ve ion)=429.10 [M+H]⁺. ¹H NMR (400 MHz, Chloroform-d) δ 8.77 (s, 1H), 8.69 (s, 1H), 7.97-7.83 (m, 2H), 7.68-7.59 (m, 2H), 7.58-7.53 (m, 2H), 7.51-7.47 (m, 1H), 7.43 (s, 1H), 7.17-7.21 (m, 2H), 5.19-4.97 (m, 1H), 4.67-4.61 (m, 1H), 3.08-2.98 (m, 2H), 2.97-2.83 (m, 2H).

### Example 151. Trans-Diastereomer Obtained as a Racemate of 4-[1-(3-fluorooxan-4-yl)-3-(4-fluorophenyl)-1H-pyrazol-4-yl]-6-phenylfuro[2,3-d]pyrimidine

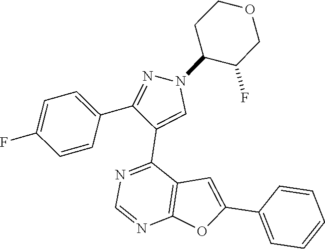

### Step 1. 3-fluorotetrahydro-2H-pyran-4-yl 4-methylbenzenesulfonate

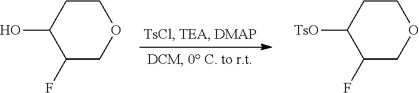 [SMILES: OC1CCOCC1F ; Cc1ccc(S(=O)(=O)OC2CCOCC2F)cc1]

To a solution of 3-fluorooxan-4-ol (600 mg, 4.995 mmol, 1 equiv.) in DCM (15 mL) at 0° C. was added TsCl (1.42 g, 7.449 mmol, 1.49 equiv.), TEA (1.39 mL, 10.000 mmol, 2.00 equiv.) and DMAP (30 mg, 0.246 mmol, 0.05 equiv.). The resulting mixture was stirred at 0° C. for 1 h, then warmed to room temperature and stirred at room temperature for 20 h. The solvent was removed under reduced pressure. The residue was purified by silica gel column chromatography, eluted with PE/EtOAc (3/1) to afford the title compound (1.13 g, 82%) as a white solid. ¹H NMR (400 MHz, Chloroform-d) δ 7.82 (d, J=8.4 Hz, 2H), 7.35 (d, J=8.1 Hz, 2H), 4.75 (m, 1H), 4.70-4.47 (m, 1H), 4.05-3.84 (m, 2H), 3.63-3.41 (m, 2H), 2.45 (s, 3H), 2.28-2.12 (m, 1H), 1.81-1.76 (m, 1H).

### Step 2. trans-Diastereomer obtained as a racemate of 4-[1-(3-fluorooxan-4-yl)-3-(4-fluorophenyl)-1H-pyrazol-4-yl]-6-phenylfuro[2,3-d]pyrimidine

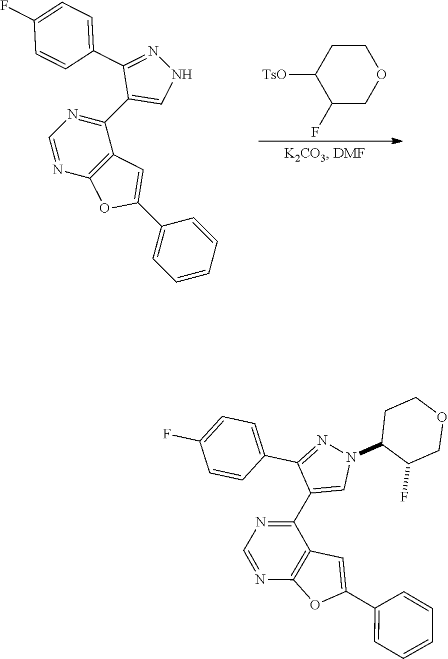

3-Fluorooxan-4-yl 4-methylbenzenesulfonate (345.3 mg, 1.259 mmol, 1 equiv.) and 4-[3-(4-fluorophenyl)-1H-pyrazol-4-yl]-6-phenylfuro[2,3-d]pyrimidine (Intermediate N, 300 mg, 0.919 mmol, 0.67 equiv.) were dissolved in DMF (6 mL) under nitrogen atmosphere. To the solution was added K₂CO₃ (232.2 mg, 1.680 mmol, 1.33 equiv.). The reaction mixture was stirred at 100° C. overnight then concentrated under reduced pressure. The residue was purified by silica gel column chromatography, eluted with PE/EtOAc (1/1) to afford the crude product. The crude was purified by Prep-HPLC to afford Example 151 (199.8 mg, 35%) as a white solid. m/z (ESI, +ve ion)=459.15 [M+H]⁺. ¹H NMR (400 MHz, Chloroform-d) δ 8.94 (s, 1H), 8.24 (s, 1H), 7.66-7.64 (m, 2H), 7.59-7.56 (m, 2H), 7.47-7.42 (m, 3H), 7.13-7.03 (m, 2H), 6.13 (s, 1H), 5.13-4.95 (m, 1H), 4.52-4.27 (m, 1H), 4.37-4.32 (m, 1H), 4.15-4.13 (m, 1H), 3.61-3.55 (m, 1H), 3.51-3.45 (m, 1H), 2.61-2.50 (m, 1H), 2.36-2.30 (m, 1H).

### Example 152. 4-{1-[(3R,4S)-3-fluorooxan-4-yl]-3-(4-fluorophenyl)-1H-pyrazol-4-yl}-6-phenylfuro[2,3-d]pyrimidine

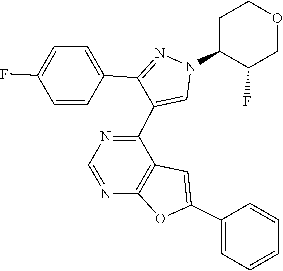 [SMILES: Fc1ccc(-c2nn([C@H]3CCOC[C@@H]3F)cc2-c2ncnc3oc(-c4ccccc4)cc23)cc1]

### Example 153. 4-{1-[(3S,4R)-3-fluorooxan-4-yl]-3-(4-fluorophenyl)-1H-pyrazol-4-yl}-6-phenylfuro[2,3-d]pyrimidine

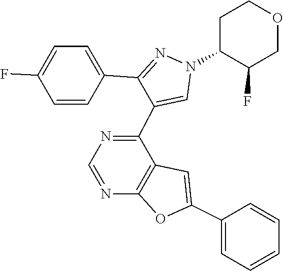 [SMILES: Fc1ccc(-c2nn([C@@H]3CCOC[C@H]3F)cc2-c2ncnc3oc(-c4ccccc4)cc23)cc1]

The 4-(3-(4-fluorophenyl)-1-((trans)-3-fluorotetrahydro-2H-pyran-4-yl)-1H-pyrazol-4-yl)-6-phenylfuro[2,3-d]pyrimidine (unknown absolute stereochemistry) (Example 152, 197.8 mg, 0.431 mmol, 1 equiv.) was separated by Prep-Chiral-HPLC with the following conditions: Column: CHIRALPAK IF, 2×25 cm, 5 μm; Mobile Phase A: Hex (0.2% FA)-HPLC, Mobile Phase B: EtOH-HPLC; Flow rate: 20 mL/min; Gradient: 30% B to 30% B in 23 min; Wavelength: 220/254 nm; RT1 (min): 16.36; RT2 (min): 19.15; Sample Solvent: MeOH:DCM=1:1-HPLC. The first product-containing fractions were collected and roto-evaporated in vacuo and lyophilized overnight to give Example 152 (76 mg, 38%) (absolute stereochemistry arbitrarily assigned) as a white solid. m/z (ESI, +ve ion)=459.10 [M+H]⁺. ¹H NMR (400 MHz, Chloroform-d) δ 8.95 (s, 1H), 8.29 (s, 1H), 7.66-7.64 (m, 2H), 7.59-7.56 (m, 2H), 7.48-7.42 (m, 3H), 7.13-7.09 (m, 2H), 6.12 (s, 1H), 5.13-4.95 (m, 1H), 4.53-4.43 (m, 1H), 4.35 (q, J=5.47, 1H), 4.17-4.11 (m, 1H), 3.62-3.45 (m, 2H), 2.54-2.53 (m, 1H), 2.36-2.31 (m, 1H). ¹⁹F NMR (376 MHz, Chloroform-d) δ −112.8, −192.9. The second product-containing fractions were collected and roto-evaporated in vacuo and lyophilized overnight to give Example 153 (absolute stereochemistry arbitrarily assigned) (72.5 mg, 37%) as a white solid. m/z (ESI, +ve ion)=459.10 [M+H]⁺. ¹H NMR (400 MHz, Chloroform-d) δ 8.93 (s, 1H), 8.20 (s, 1H), 7.67-7.64 (m, 2H), 7.60-7.55 (m, 2H), 7.48-7.39 (m, 3H), 7.13-7.07 (m, 2H), 6.14 (s, 1H), 5.13-4.95 (m, 1H), 4.51-4.42 (m, 1H), 4.35 (dd, J=11.2, 4.8 Hz, 1H), 4.17-4.11 (m, 1H), 3.58-3.55 (m, 1H), 3.51-3.45 (m, 1H), 2.54-2.53 (m, 1H), 2.36-2.30 (m, 1H).

### Example 155. 3-{4-[6-(1-acetyl-1,4,5,6-tetrahydropyridin-3-yl)furo[2,3-d]pyrimidin-4-yl]-3-(4-fluorophenyl)-1H-pyrazol-1-yl}-1λ6-thietane-1,1-dione

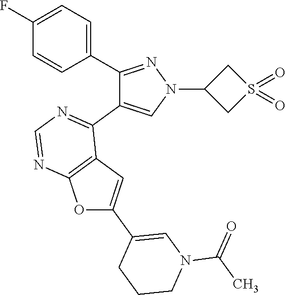 [SMILES: CC(=O)N1C=C(c2cc3c(-c4cn(C5CS(=O)(=O)C5)nc4-c4ccc(F)cc4)ncnc3o2)CCC1]

### Step 1. tert-Butyl 5-(4-(1-(1,1-dioxidothietan-3-yl)-3-(4-fluorophenyl)-1H-pyrazol-4-yl)furo[2,3-d]pyrimidin-6-yl)-3,4-dihydropyridine-1 (2H)-carboxylate

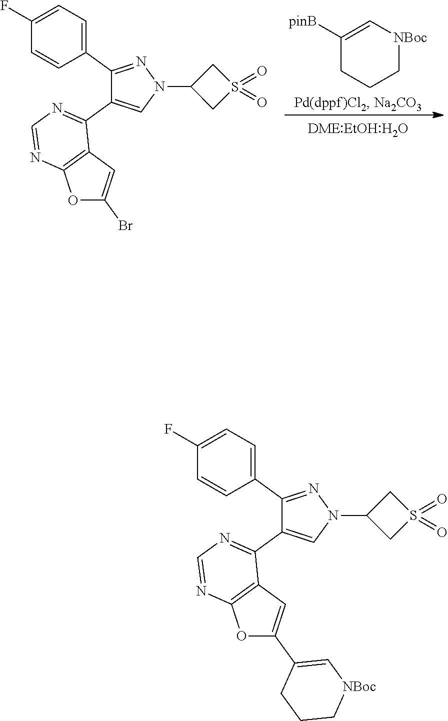

3-(4-{6-Bromofuro[2,3-d]pyrimidin-4-yl}-3-(4-fluorophenyl)pyrazol-1-yl)-1,6-thietane-1,1-dione (Intermediate S, 100 mg, 216 μmol), tert-butyl 5-(4,4,5,5-tetramethyl-1,3,2-dioxaborolan-2-yl)-3,4-dihydropyridine-1 (2H)-carboxylate (80 mg, 259 μmol, 1.2 equiv.), sodium carbonate (69 mg, 648 μmol, 3.0 equiv.) and 1,1′-bis(diphenylphosphino)ferrocene dichloropalladium (II) (17 mg, 21.6 μmol, 0.1 equiv.) were dissolved in a mixture of DME (1.40 mL), EtOH (700 μL), and H₂O (350 μL). The mixture was degassed with nitrogen and heated to 90° C. for 16 hours. After cooling, the reaction mixture was diluted with water and extracted with EtOAc three times. The combined organic layers were washed with brine, dried over Na₂SO₄, filtered, and concentrated under reduced pressure. The crude material was purified by column chromatography (50-100% EtOAc in heptanes, a gradient elution) to afford the title compound (100 mg, 69%) as solid. m/z (ESI, +ve ion)=566.4 [M+H]⁺.

### Step 2. 3-(3-(4-fluorophenyl)-4-(6-(1,4,5,6-tetrahydropyridin-3-yl)furo[2,3-d]pyrimidin-4-yl)-1H-pyrazol-1-yl)thietane 1,1-dioxide

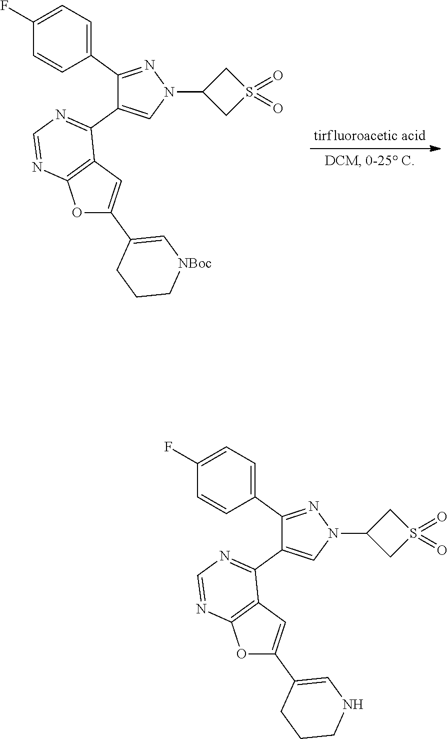 [SMILES: CC(C)(C)OC(=O)N1C=C(c2cc3c(-c4cn(C5CS(=O)(=O)C5)nc4-c4ccc(F)cc4)ncnc3o2)CCC1 ; O=S1(=O)CC(n2cc(-c3ncnc4oc(C5=CNCCC5)cc34)c(-c3ccc(F)cc3)n2)C1]

Tert-butyl 5-(4-(1-(1,1-dioxidothietan-3-yl)-3-(4-fluorophenyl)-1H-pyrazol-4-yl)furo[2,3-d]pyrimidin-6-yl)-3,4-dihydropyridine-1 (2H)-carboxylate (100 mg, 177 μmol) was dissolved in DCM (442 μL) and the mixture was cooled to 0° C. Trifluoroacetic acid (438 μL, 5.66 mmol, 32.0 equiv.) was added and the mixture was stirred at room temperature for 1 hour. The reaction mixture was evaporated, diluted with DCM. Activated charcoal (80 mg) was added to the mixture. The resulting mixture was stirred at room temperature for 30 minutes, filtered over Celite and washed with DCM. The filtrate was concentrated under reduced pressure to afford the title compound (65.5 mg, 80%) as a solid. ¹H NMR (400 MHz, DMSO-d₆) δ 8.60 (s, 1H), 8.50 (s, 1H), 7.61-7.54 (m, 2H), 7.24-7.16 (m, 3H), 6.55-6.50 (m, 1H), 5.76 (s, 1H), 5.54-5.44 (m, 1H), 4.93-4.78 (m, 4H), 3.19-3.13 (m, 2H), 2.15 (t, J=6.1 Hz, 2H), 1.84-1.75 (m, 2H). m/z (ESI, +ve ion)=466.3 [M+H]⁺.

### Step 3. 3-{4-[6-(1-acetyl-1,4,5,6-tetrahydropyridin-3-yl)furo[2,3-d]pyrimidin-4-yl]-3-(4-fluorophenyl)-1H-pyrazol-1-yl}-16-thietane-1,1-dione (155c)

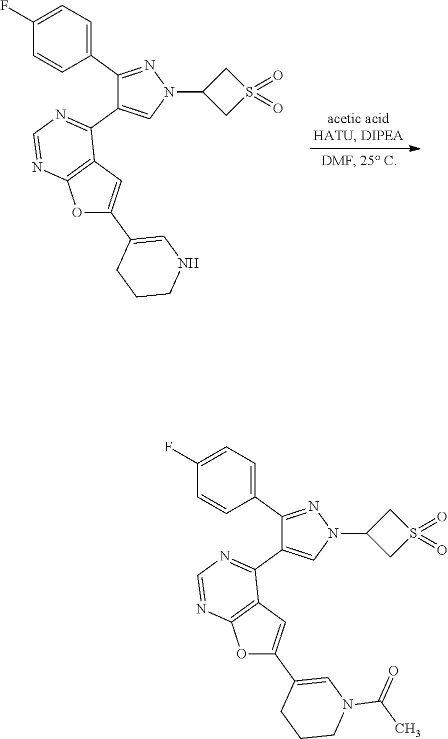 [SMILES: O=S1(=O)CC(n2cc(-c3ncnc4oc(C5=CNCCC5)cc34)c(-c3ccc(F)cc3)n2)C1 ; CC(=O)N1C=C(c2cc3c(-c4cn(C5CS(=O)(=O)C5)nc4-c4ccc(F)cc4)ncnc3o2)CCC1]

Acetic acid (22.4 μL, 387 μmol, 4.0 equiv.) and N,N-diisopropylethylamine (67.4 μL, 387 μmol, 4.0 equiv.) were added to a solution of 3-(3-(4-fluorophenyl)-4-(6-(1,4,5,6-tetrahydropyridin-3-yl)furo[2,3-d]pyrimidin-4-yl)-1H-pyrazol-1-yl)thietane 1,1-dioxide (45.0 mg, 96.7 μmol) in DMF (483 μL) at room temperature. HATU (55.7 mg, 145 μmol, 1.5 equiv.) was added to the mixture. The reaction mixture was stirred at room temperature for 1 h. The reaction mixture purified by prep-HPLC (Buchi Pure C850, Phenomenex Luna C18, 10 μm, 250 mm×21.2 mm, eluent: MeCN with 10 mM ammonium formate in water, a gradient of 10-100%) to afford the title compound (13.5 mg, 27%) as a solid. ¹H NMR (400 MHz, DMSO-d₆) δ 8.72 (d, J=3.9 Hz, 1H), 8.69 (s, 1H), 7.62-7.54 (m, 3H), 7.22 (t, J=8.4 Hz, 2H), 6.50 (d, J=3.7 Hz, 1H), 5.54-5.45 (m, 1H), 4.94-4.81 (m, 4H), 3.73-3.60 (m, 2H), 2.32 and 2.21 (s, 3H) 2.29 (d, J=5.8 Hz, 2H), 1.98-1.84 (m, 2H).

### Example 156. 4-[3-(4-fluorophenyl)-1-(3-fluoropropyl)-1H-pyrazol-4-yl]-6-phenylfuro[2,3-d]pyrimidine

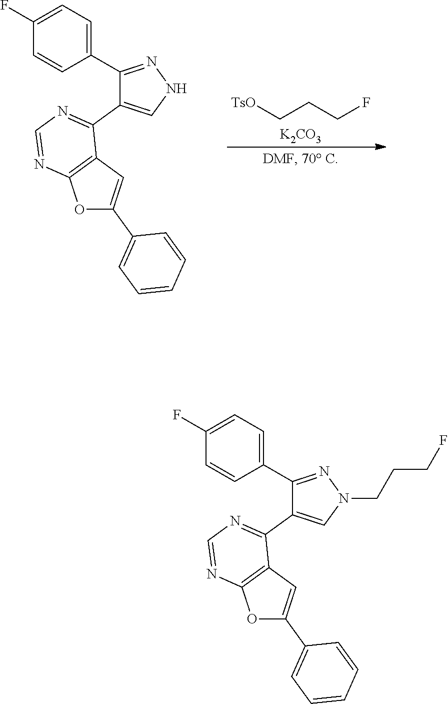

3-Fluoropropyl 4-methylbenzenesulfonate (79.8 mg, 337 μmol, 1.2 equiv.) was added to a mixture of 4-[3-(4-fluorophenyl)-1H-pyrazol-4-yl]-6-phenylfuro[2,3-d]pyrimidine (Intermediate N, 100 mg, 281 μmol) and potassium carbonate (79.1 mg, 561 μmol, 2.0 equiv.) in DMF (2.80 mL). The mixture was stirred at 70° C. for 72 hours, then diluted with water (20 mL) and extracted with EtOAc (3×10 mL). The combined organic layers were washed with water, brine, dried (Na₂SO₄), filtered and concentrated. The crude material was purified by column chromatography (EtOAc in hexanes, 0-30%, a gradient elution) and followed by preparative TLC (70% Et₂O in hexanes, isocratic elution) to afford the title compound (49.0 mg, 42%) as a solid. ¹H NMR (400 MHz, DMSO-d₆) δ 8.77 (s, 1H), 8.68 (s, 1H), 7.95-7.90 (m, 2H), 7.59 (ddd, J=14.8, 8.3, 6.0 Hz, 4H), 7.53-7.47 (m, 1H), 7.44 (s, 1H), 7.19 (t, J=8.9 Hz, 2H), 4.59 (dt, J=47.2, 5.7 Hz, 2H), 4.40 (t, J=7.1 Hz, 2H), 2.41-2.27 (m, 2H). m/z (ESI, +ve ion)=417.4 [M+H]⁺.

### Example 157. 4-[1-(3,3-difluoropropyl)-3-(4-fluorophenyl)-1H-pyrazol-4-yl]-2-phenylfuro[2,3-b]pyridine

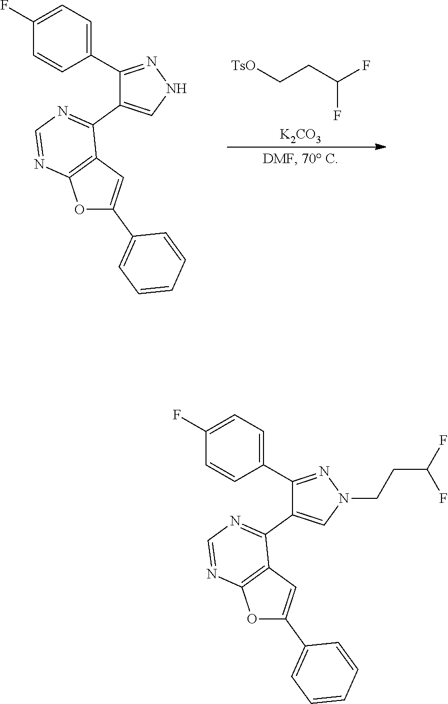 [SMILES: Fc1ccc(-c2n[nH]cc2-c2ncnc3oc(-c4ccccc4)cc23)cc1 ; "K2CO3" ; Cc1ccc(S(=O)(=O)OCCC(F)F)cc1 ; Fc1ccc(-c2nn(CCC(F)F)cc2-c2ncnc3oc(-c4ccccc4)cc23)cc1]

3,3-Difluoropropyl 4-methylbenzenesulfonate (105 mg, 421 μmol, 1.5 equiv.) was added to a mixture of 4-[3-(4-Fluorophenyl)-1H-pyrazol-4-yl]-6-phenylfuro[2,3-d]pyrimidine (Intermediate N, 100 mg, 281 μmol) and potassium carbonate (79.1 mg, 561 μmol, 2.0 equiv.) in DMF (2.80 mL). The mixture was stirred for 15 hours at 70° C., then diluted with water (20 mL) and extracted with EtOAc (3×100 mL). The combined organic layers were washed with water, brine, then dried (Na₂SO₄), filtered and concentrated. The crude material was purified by column chromatography (0-30% EtOAc in hexanes, a gradient elution) to provide the title compound (27.0 mg, 22%) as a solid. ¹H NMR (400 MHz, DMSO-d₆) δ 8.77 (s, 1H), 8.70 (s, 1H), 7.92 (d, J=7.3 Hz, 2H), 7.65-7.54 (m, 4H), 7.50 (t, J=7.3 Hz, 1H), 7.44 (s, 1H), 7.20 (t, J=8.9 Hz, 2H), 6.46-6.11 (m, 1H), 4.46 (t, J=7.1 Hz, 2H), 2.65-2.52 (m, 2H). m/z (ESI, +ve ion)=435.6 [M+H]⁺.

### Example 158. 3-[3-(4-fluorophenyl)-4-(6-phenylfuro[2,3-d]pyrimidin-4-yl)-1H-pyrazol-1-yl]-2,2-dimethylpropanamide

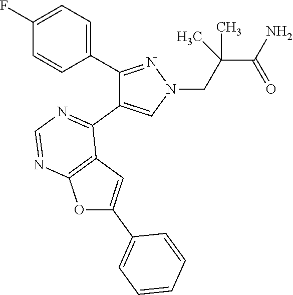 [SMILES: CC(C)(Cn1cc(-c2ncnc3oc(-c4ccccc4)cc23)c(-c2ccc(F)cc2)n1)C(N)=O]

### Step 1. Ethyl 3-(3-(4-fluorophenyl)-4-(6-phenylfuro[2,3-d]pyrimidin-4-yl)-1H-pyrazol-1-yl)-2,2-dimethylpropanoate

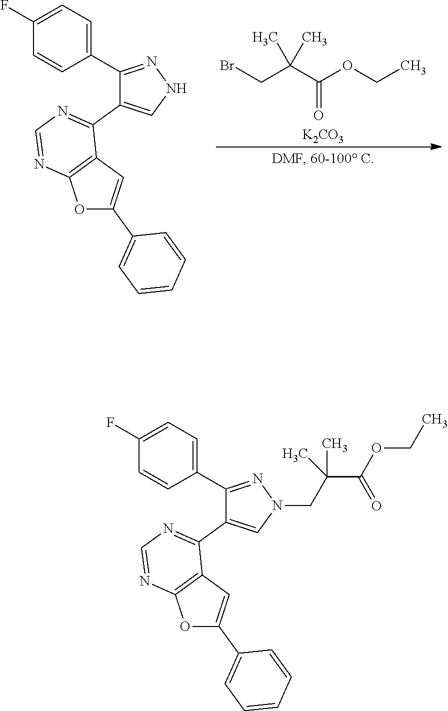

4-[3-(4-Fluorophenyl)-1H-pyrazol-4-yl]-6-phenylfuro[2,3-d]pyrimidine (Intermediate N, 200 mg, 561 μmol) and potassium carbonate (198 mg, 1.40 mmol, 2.5 equiv.) were suspended in DMF (2.80 mL). After 5 minutes, ethyl 3-bromo-2,2-dimethylpropanoate (148 mg, 673 μmol, 0.6 equiv.) was added. The reaction mixture was stirred at 60° C. for 72 hours. The reaction was heated to 100° C. and stirred for an additional 24 hours. Additional ethyl 3-bromo-2,2-dimethylpropanoate (74.1 mg, 337 μmol) was added and the reaction was left to stir at 100° C. for 24 hours. H₂O (10 mL) and EtOAc (10 mL) were added and the aqueous layer was extracted with additional EtOAc (3×20 mL). The combined organic layers were washed with a saturated solution of Na₂S₂O₃ (10 mL), H₂O (10 mL), brine (10 mL), dried over Na₂SO₄, filtered and evaporated. The crude material was purified by column chromatography (50-100% EtOAc in hexanes, a gradient elution) to provide the title compound (155 mg, 57%) as an oil. m/z (ESI, +ve ion)=485.4 [M+H]⁺.

### Step 2. 3-(3-(4-Fluorophenyl)-4-(6-phenylfuro[2,3-d]pyrimidin-4-yl)-1H-pyrazol-1-yl)-2,2-dimethylpropanoic acid

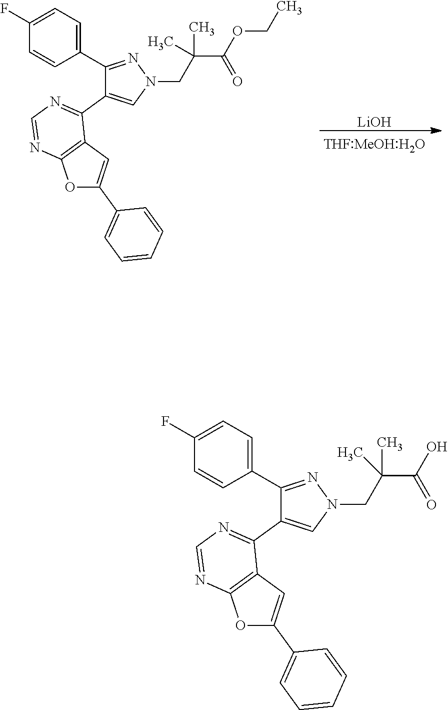 [SMILES: CCOC(=O)C(C)(C)Cn1cc(-c2ncnc3oc(-c4ccccc4)cc23)c(-c2ccc(F)cc2)n1 ; "LiOH" ; CC(C)(Cn1cc(-c2ncnc3oc(-c4ccccc4)cc23)c(-c2ccc(F)cc2)n1)C(=O)O]

Ethyl 3-(3-(4-fluorophenyl)-4-(6-phenylfuro[2,3-d]pyrimidin-4-yl)-1H-pyrazol-1-yl)-2,2-dimethylpropanoate (155 mg, 320 μmol) was dissolved in a mixture of THF (2.30 mL), MeOH (580 μL), and water (1.15 mL). Lithium hydroxide (39.1 mg, 1.60 mmol) was added and the mixture was stirred at room temperature for 2 hours. The mixture was diluted with water (5 mL) and EtOAc (5 mL). The layers were separated, and the aqueous layer was washed with EtOAc (2×5 mL). The aqueous layer was adjusted to pH 4 using 1 M HCl aqueous solution. The aqueous layer was extracted with EtOAc (3×5 mL). The combined organic layers were dried over Na₂SO₄, filtered, and evaporated to afford the title compound (146 mg, quant) as a solid. m/z (ESI, +ve ion)=457.4 [M+H]⁺.

### Step 3. 3-[3-(4-fluorophenyl)-4-(6-phenylfuro[2,3-d]pyrimidin-4-yl)-1H-pyrazol-1-yl]-2,2-dimethylpropanamide

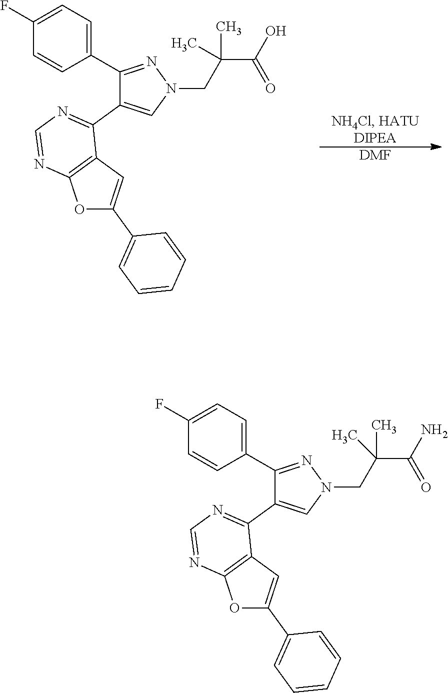

N,N-Diisopropylethylamine (139 μL, 800 μmol) and ammonium chloride (68.4 mg, 1.28 mmol) were added to a solution of 3-(3-(4-fluorophenyl)-4-(6-phenylfuro[2,3-d]pyrimidin-4-yl)-1H-pyrazol-1-yl)-2,2-dimethylpropanoic acid (146 mg, 320 μmol) in DMF (1.60 mL) at room temperature. Then, HATU (184 mg, 480 μmol) was added. The reaction mixture was stirred at room temperature for 1 hour. The material was directly purified by prep-HPLC (Buchi Pure C850, Phenomenex Luna C18, 10 μm, 250 mm×21.2 mm, eluent: MeCN in 10 mM ammonium formate in water, a gradient of 20-80%) to afford the title compound (35.5 mg, 24%) as a solid. ¹H NMR (400 MHz, DMSO-d₆) δ 8.78 (s, 1H), 8.47 (s, 1H), 7.93-7.87 (m, 2H), 7.62-7.53 (m, 4H), 7.53-7.47 (m, 1H), 7.30 (s, 1H), 7.28 (s, 1H), 7.23-7.16 (m, 2H), 7.12 (s, 1H), 4.39 (s, 2H), 1.20 (s, 6H). m/z (ESI, +ve ion)=456.5 [M+H]⁺.

### Example 161. 3-[3-(4-fluorophenyl)-4-(6-phenylfuro[2,3-d]pyrimidin-4-yl)-1H-pyrazol-1-yl]-1-imino-1λ6-thiolan-1-one

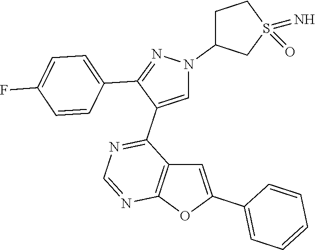 [SMILES: N=S1(=O)CCC(n2cc(-c3ncnc4oc(-c5ccccc5)cc34)c(-c3ccc(F)cc3)n2)C1]

### Step 1. 4-(3-(4-fluorophenyl)-1-(tetrahydrothiophen-3-yl)-1H-pyrazol-4-yl)-6-phenylfuro[2,3-d]pyrimidine

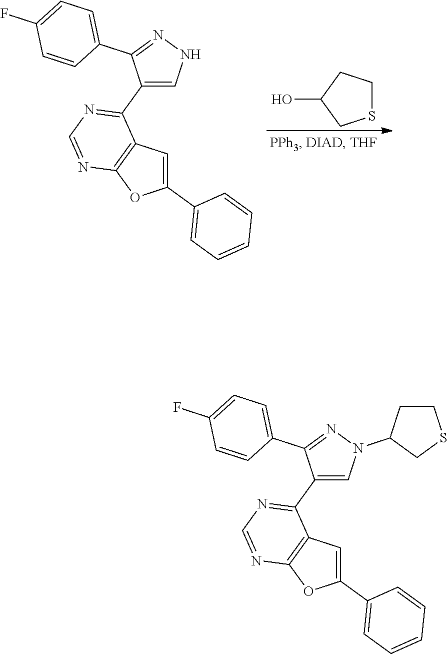 [SMILES: Fc1ccc(-c2n[nH]cc2-c2ncnc3oc(-c4ccccc4)cc23)cc1 ; OC1CCSC1 ; Fc1ccc(-c2nn(C3CCSC3)cc2-c2ncnc3oc(-c4ccccc4)cc23)cc1]

To a solution of 4-[3-(4-Fluorophenyl)-1H-pyrazol-4-yl]-6-phenylfuro[2,3-d]pyrimidine (Intermediate N, 163.2 mg, 0.458 mmol, 1 equiv.), thiolan-3-ol (62.4 mg, 0.599 mmol, 1.31 equiv.) and Ph₃P (196.7 mg, 0.750 mmol, 1.64 equiv.) in dry THF (10 mL) at 0° C. under nitrogen atmosphere was added DIAD (0.2 mL, 1.009 mmol, 2.20 equiv.). The resulting mixture was warmed to room temperature and stirred for 3 h. The mixture was concentrated under reduced pressure. The residue was purified by Prep-TLC (PE/EtOAc=3/2) to afford the title compound (crude, 193 mg, 95%) as an orange oil. m/z (ESI, +ve ion)=443.10 [M+H]⁺.

### Step 2. 3-[3-(4-fluorophenyl)-4-(6-phenylfuro[2,3-d]pyrimidin-4-yl)-1H-pyrazol-1-yl]-1-imino-1λ6-thiolan-1-one

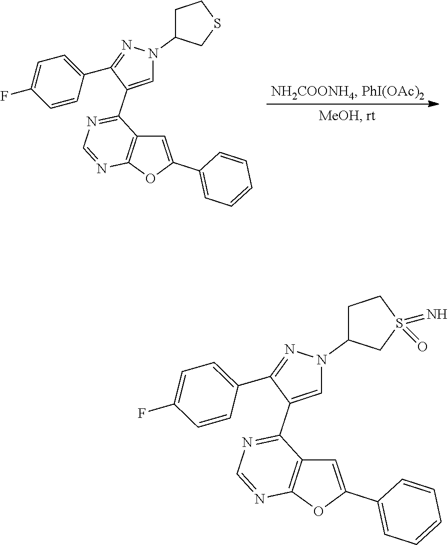 [SMILES: Fc1ccc(-c2nn(C3CCSC3)cc2-c2ncnc3oc(-c4ccccc4)cc23)cc1 ; N=S1(=O)CCC(n2cc(-c3ncnc4oc(-c5ccccc5)cc34)c(-c3ccc(F)cc3)n2)C1]

To the mixture of 4-(3-(4-fluorophenyl)-1-(tetrahydrothiophen-3-yl)-1H-pyrazol-4-yl)-6-phenylfuro[2,3-d]pyrimidine (78.3 mg, 0.177 mmol, 1 equiv.), ammonium carbamate (27.49 mg, 0.352 mmol, 1.99 equiv.) and PhI(OAc)₂ (142.48 mg, 0.442 mmol, 2.50 equiv.) was added MeOH (4 mL) under nitrogen atmosphere. The resulting mixture was stirred at room temperature overnight and then at 40° C. for an additional 3 h. The solvent was removed under reduced pressure. The residue was purified by prep-HPLC and prep-TLC to afford Example 161 (15.0 mg, 18%) as a white solid. m/z (ESI, +ve ion)=474.15 [M+H]⁺. ¹H NMR (400 MHz, Chloroform-d) δ 8.94 (s, 1H), 8.32 (s, 1H), 7.67-7.65 (m, 2H), 7.55 (dd, J=8.4, 5.2 Hz, 2H), 7.48-7.41 (m, 3H), 7.11 (t, J=8.6 Hz, 2H), 6.15 (s, 1H), 5.34 (s, 1H), 3.90-3.79 (m, 2H), 3.72-3.66 (m, 1H), 3.42-3.35 (m, 1H), 2.97-2.91 (m, 2H), 2.74 (brs, 1H).

### Example 162. (1s,3s)-3-{[3-(4-fluorophenyl)-4-(6-phenylfuro[2,3-d]pyrimidin-4-yl)-1H-pyrazol-1-yl]methyl}-1-methylcyclobutan-1-ol

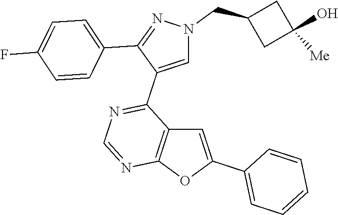 [SMILES: C[C@]1(O)C[C@@H](Cn2cc(-c3ncnc4oc(-c5ccccc5)cc34)c(-c3ccc(F)cc3)n2)C1]

### Step 1. 3-(hydroxymethyl)-1-methylcyclobutan-1-ol

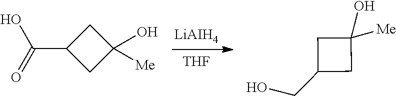

To a stirred solution of LiAlH₄ (291.60 mg, 7.685 mmol, 5 equiv.) in THF (1 mL) at 0° C. under nitrogen atmosphere was added 3-hydroxy-3-methylcyclobutane-1-carboxylic acid (200 mg, 1.537 mmol, 1 equiv.) in THF (0.5 mL) dropwise. The resulting mixture was stirred for 16 h at 65° C. under nitrogen atmosphere. The mixture was cooled to room temperature before being quenched by the addition of acetone (10 mL) at 0° C. To the above mixture was added H₂O (10 mL) and 15% aqueous solution of NaOH (10 mL) dropwise over 5 min at 0° C. The resulting mixture was stirred for an additional 2 h at 0° C. The mixture was filtered and the filter cake was washed with THF (2×10 mL). The filtrate was concentrated under reduced pressure to afford the title compound (170 mg, 95%) as a yellow oil that was used directly in next step.

### Step 2. (3-hydroxy-3-methylcyclobutyl)methyl 4-methylbenzenesulfonate

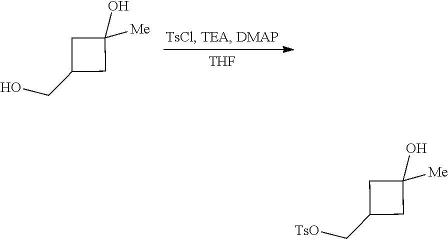

To a stirred solution of 3-(hydroxymethyl)-1-methylcyclobutan-1-ol (200 mg, 1.722 mmol, 1 equiv.) and TEA (348.46 mg, 3.444 mmol, 2 equiv.) in DCM (4 mL) was added TsCl (361.06 mg, 1.894 mmol, 1.1 equiv.) at room temperature under nitrogen. The resulting mixture was stirred for 16 h at room temperature under nitrogen then concentrated under reduced pressure. The residue was purified by silica gel column chromatography, eluted with PE/EtOAc (1/1) to afford the title compound (210 mg, 45%) as a colorless oil. No MS on LCMS. ¹H NMR (400 MHz, Chloroform-d) δ 7.84-7.78 (m, 2H), 7.41-7.34 (m, 2H), 4.06-4.00 (m, 2H), 2.48 (s, 3H), 2.26-2.10 (m, 3H), 1.92-1.75 (m, 2H), 1.37 (s, 3H).

### Step 3. (1s,3s)-3-{[3-(4-fluorophenyl)-4-(6-phenylfuro[2,3-d]pyrimidin-4-yl)-1H-pyrazol-1-yl]methyl}-1-methylcyclobutan-1-ol

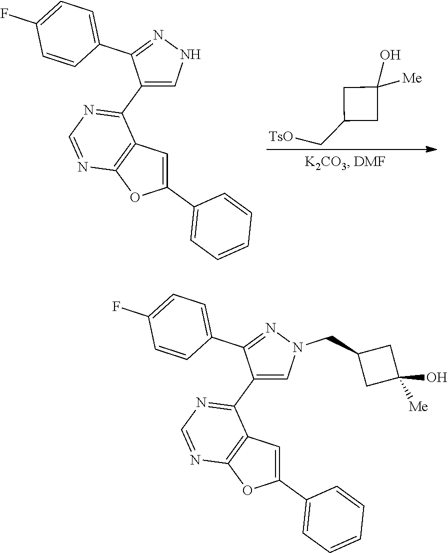 [SMILES: Fc1ccc(-c2n[nH]cc2-c2ncnc3oc(-c4ccccc4)cc23)cc1 ; Cc1ccc(S(=O)(=O)OCC2CC(C)(O)C2)cc1 ; C[C@]1(O)C[C@@H](Cn2cc(-c3ncnc4oc(-c5ccccc5)cc34)c(-c3ccc(F)cc3)n2)C1]

To a stirred solution of 4-[3-(4-Fluorophenyl)-1H-pyrazol-4-yl]-6-phenylfuro[2,3-d]pyrimidine (Intermediate N, 200 mg, 0.561 mmol, 1.00 equiv.) in DMF (3.3 mL) at room temperature under nitrogen was added K₂CO₃ (155.13 mg, 1.122 mmol, 2 equiv.). The resulting mixture was stirred for 15 min at room temperature under nitrogen. To the above mixture was added (3-hydroxy-3-methylcyclobutyl)methyl 4-methylbenzenesulfonate (182.07 mg, 0.673 mmol, 1.2 equiv.) at room temperature. The resulting mixture was stirred for additional 16 h at room temperature then quenched by the addition of water (30 mL) at 0° C. The mixture was extracted with EtOAc (3×30 mL). The combined organic layers were washed with brine (3×30 mL) and dried over anhydrous Na₂SO₄. After filtration, the filtrate was concentrated under reduced pressure. The crude product was purified by Prep-HPLC with the following conditions (Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 48% B to 63% B in 8 min, 63% B; Wavelength: 254 nm; RT1 (min): 6.52 to afford Example 162 (25.3 mg, 10%) as a white solid. m/z (ESI, +ve ion)=455.20 [M+H]⁺. ¹H NMR (400 MHz, Chloroform-d) δ 9.03 (s, 1H), 8.55 (s, 1H), 7.62-7.60 (m, 2H), 7.56-7.52 (m, 2H), 7.50-7.45 (m, 3H), 7.16-7.11 (m, 2H), 5.98 (s, 1H), 4.37 (d, J=7.2 Hz, 2H), 2.61-2.52 (m, 1H), 2.37-2.31 (m, 2H), 2.09-2.01 (m, 2H), 1.44 (s, 3H).

### Example 163. 4-{3-(4-fluorophenyl)-1-[(1s,3s)-3-(methanesulfonyl)cyclobutyl]-1H-pyrazol-4-yl}-6-phenylfuro[2,3-d]pyrimidine

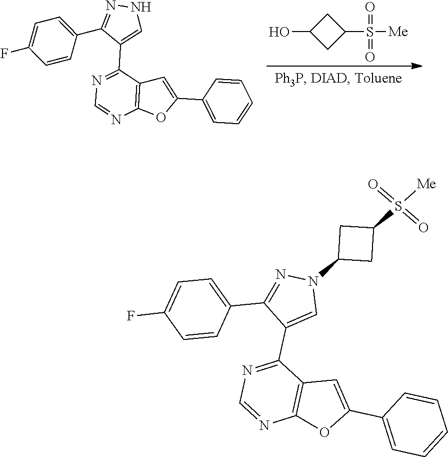 [SMILES: Fc1ccc(-c2n[nH]cc2-c2ncnc3oc(-c4ccccc4)cc23)cc1 ; CS(=O)(=O)C1CC(O)C1 ; CS(=O)(=O)[C@H]1C[C@@H](n2cc(-c3ncnc4oc(-c5ccccc5)cc34)c(-c3ccc(F)cc3)n2)C1]

To a stirred solution of 3-(methylsulfonyl)cyclobutanol (99 mg, 0.657 mmol, 1 equiv.), 4-[3-(4-Fluorophenyl)-1H-pyrazol-4-yl]-6-phenylfuro[2,3-d]pyrimidine (Intermediate N, 233.89 mg, 0.657 mmol, 1 equiv.) and PPh₃ (258.52 mg, 0.986 mmol, 1.5 equiv.) in toluene (8 mL) at 0° C. under nitrogen was added DIAD (265.73 mg, 1.314 mmol, 2 equiv.). The resulting mixture was stirred for 5 h at 100° C. then cooled down to room temperature. The resulting mixture was diluted with water (30 mL) and extracted with EtOAc (3×30 mL). The combined organic layers were washed with brine (2×30 mL) and dried over anhydrous Na₂SO₄. After filtration, the filtrate was concentrated under reduced pressure. The crude product was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 46% B to 56% B in 8 min, 56% B; Wavelength: 254 nm; RT1 (min): 7) to afford Example 163 (43.7 mg, 14%) as a white solid. m/z (ESI, +ve ion)=489.15 [M+H]⁺. ¹H NMR (400 MHz, Chloroform-d) δ 8.90 (s, 1H), 8.28 (s, 1H), 7.68-7.66 (m, 2H), 7.56-7.54 (m, 2H), 7.47-7.39 (m, 3H), 7.11-7.06 (m, 2H), 6.20 (s, 1H), 5.01-4.97 (m, 1H), 3.75-3.71 (m, 1H), 3.28-3.20 (m, 2H), 3.09-3.02 (m, 2H), 2.95 (s, 3H).

### Example 164. 3-{3-(4-fluorophenyl)-4-[6-(pyridin-3-yl)furo[2,3-d]pyrimidin-4-yl]-1H-pyrazol-1-yl}-1λ6-thietane-1,1-dione

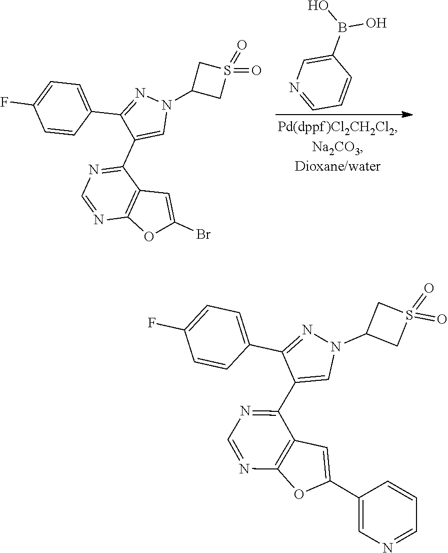 [SMILES: O=S1(=O)CC(n2cc(-c3ncnc4oc(Br)cc34)c(-c3ccc(F)cc3)n2)C1 ; OB(O)c1cccnc1 ; O=S1(=O)CC(n2cc(-c3ncnc4oc(-c5cccnc5)cc34)c(-c3ccc(F)cc3)n2)C1]

To a stirred solution of 3-(4-{6-bromofuro[2,3-d]pyrimidin-4-yl}-3-(4-fluorophenyl)pyrazol-1-yl)-1λ6-thietane-1,1-dione (Intermediate S, 120 mg, 0.259 mmol, 1 equiv.) and pyridin-3-ylboronic acid (70.05 mg, 0.570 mmol, 2.2 equiv.) in dioxane (3 mL) and water (0.75 mL) at room temperature were added Pd(dppf)Cl₂·CH₂Cl₂ (21.10 mg, 0.026 mmol, 0.1 equiv.) and Na₂CO₃ (82.36 mg, 0.777 mmol, 3 equiv.). The resulting mixture was stirred for 2 h at 100° C. under nitrogen atmosphere and then cooled to room temperature. The reaction was quenched by the addition of water (10 mL) and extracted with EtOAc (3×10 mL). The combined organic layers were washed with brine (3×5 mL) and dried over anhydrous Na₂SO₄. After filtration, the filtrate was concentrated under reduced pressure. The resulting solution was decolorized by the addition of active carbon. The crude product (120 mg) was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 33% B to 43% B in 8 min, 43% B; Wavelength: 254 nm; RT1 (min): 7) to afford Example 164 (46.2 mg, 38%) as a white solid. m/z (ESI, +ve ion)=462.05 [M+H]⁺. ¹H NMR (400 MHz, DMSO-d₆) δ 9.21-9.12 (m, 1H), 8.84 (s, 1H), 8.78 (s, 1H), 8.72-8.63 (m, 1H), 8.31-8.24 (m, 1H), 7.70-7.55 (m, 4H), 7.27-7.17 (m, 2H), 5.59-5.47 (m, 1H), 4.99-4.85 (m, 4H).

### Example 165. 3-{3-(4-fluorophenyl)-4-[6-(pyridin-4-yl)furo[2,3-d]pyrimidin-4-yl]-1H-pyrazol-1-yl}-1λ6-thietane-1,1-dione

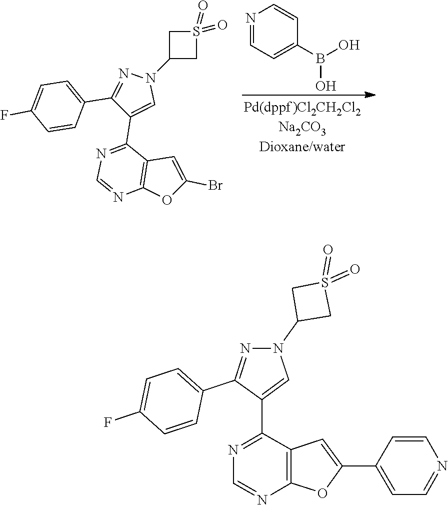

To a stirred solution of 3-(4-{6-bromofuro[2,3-d]pyrimidin-4-yl}-3-(4-fluorophenyl)pyrazol-1-yl)-1λ6-thietane-1,1-dione (Intermediate S, 120 mg, 0.259 mmol, 1 equiv.) and pyridin-4-ylboronic acid (70.05 mg, 0.570 mmol, 2.2 equiv.) in 1,4-dioxane (3.2 mL) and H₂O (0.8 mL) at room temperature were added Pd(dppf)Cl₂·CH₂Cl₂ (21.10 mg, 0.026 mmol, 0.1 equiv.) and Na₂CO₃ (82.36 mg, 0.777 mmol, 3.0 equiv.). The resulting mixture was stirred for 2 h at 100° C. under nitrogen atmosphere and then cooled to room temperature. The reaction was quenched by the addition of water (10 mL) and extracted with EtOAc (3×10 mL). The combined organic layers were washed with brine (3×10 mL) and dried over anhydrous Na₂SO₄. After filtration, the filtrate was concentrated under reduced pressure. The resulting solution was decolorized by the addition of active carbon. The crude product (120 mg) was purified by Prep-HPLC with the following conditions (Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 38% B to 43% B in 8 min, 43% B; Wavelength: 254 nm; RT1 (min): 7.53) to afford Example 165 (37.1 mg, 31%) as a white solid. m/z (ESI, +ve ion)=462.10 [M+H]⁺. ¹H NMR (400 MHz, DMSO-d₆) δ 8.87 (s, 1H), 8.83-8.72 (m, 3H), 7.89-7.80 (m, 2H), 7.72 (s, 1H), 7.68-7.59 (m, 2H), 7.28-7.10 (m, 2H), 5.68-5.37 (m, 1H), 5.14-4.77 (m, 4H).

### Example 166. (3R)-3-[3-(4-fluorophenyl)-4-(6-phenylfuro[2,3-d]pyrimidin-4-yl)-1H-pyrazol-1-yl]-1-methylpyrrolidin-2-one

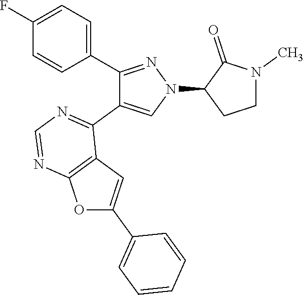 [SMILES: CN1CC[C@@H](n2cc(-c3ncnc4oc(-c5ccccc5)cc34)c(-c3ccc(F)cc3)n2)C1=O]

### Example 167. (3S)-3-[3-(4-fluorophenyl)-4-(6-phenylfuro[2,3-d]pyrimidin-4-yl)-1H-pyrazol-1-yl]-1-methylpyrrolidin-2-one

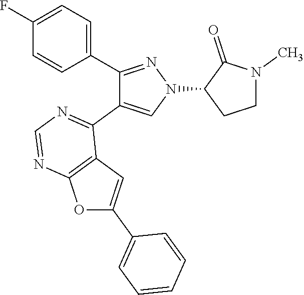 [SMILES: CN1CC[C@H](n2cc(-c3ncnc4oc(-c5ccccc5)cc34)c(-c3ccc(F)cc3)n2)C1=O]

The mixture of Example 148 was separated by chiral-HPLC using the following conditions: chiral separation conditions: column: Lux Amylos-2, 5 μm, 10×250 mm; mode: Isocratic; mobile phase: 60% IPA+10 mM ammonium formate in water/60% supercritical CO₂; flow rate: 10 mL/min; backpressure: 150 bar; column temperature: 40° C.; run time (min): 8, to afford Example 166 (15.8 mg, 12%) (absolute stereochemistry arbitrarily assigned) followed by Example 167 (14.3 mg, 11%) (absolute stereochemistry arbitrarily assigned) as solids. Example 166. ¹H NMR (400 MHz, DMSO-d₆) δ 8.79 (s, 1H), 8.72 (s, 1H), 7.92-7.88 (m, 2H), 7.59-7.53 (m, 4H), 7.52-7.47 (m, 1H), 7.35 (s, 1H), 7.22-7.16 (m, 2H), 5.29 (t, J=9.0 Hz, 1H), 3.61-3.54 (m, 1H), 3.53-3.44 (m, 1H), 2.86 (s, 3H), 2.70-2.60 (m, 2H). m/z (ESI, +ve ion)=454.4 [M+H]⁺. Example 167. ¹H NMR (400 MHz, DMSO-d₆) δ 8.79 (s, 1H), 8.72 (s, 1H), 7.93-7.86 (m, 2H), 7.62-7.53 (m, 4H), 7.53-7.47 (m, 1H), 7.35 (s, 1H), 7.23-7.14 (m, 2H), 5.29 (t, J=9.0 Hz, 1H), 3.57 (td, J=9.2, 3.1 Hz, 1H), 3.53-3.44 (m, 1H), 2.86 (s, 3H), 2.68-2.57 (m, 2H). m/z (ESI, +ve ion)=454.4 [M+H]⁺.

### Example 171. 3-[3-(4-fluorophenyl)-4-(8-phenyl-9H-purin-6-yl)-1H-pyrazol-1-yl]-1λ6-thiolane-1,1-dione

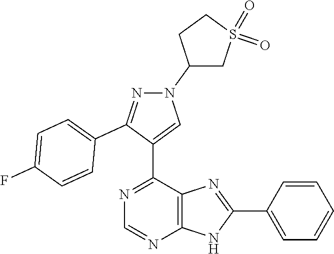 [SMILES: O=S1(=O)CCC(n2cc(-c3ncnc4[nH]c(-c5ccccc5)nc34)c(-c3ccc(F)cc3)n2)C1]

### Step 1. 3-[3-(4-fluorophenyl)-4-{9-[(4-methoxyphenyl)methyl]-8-phenylpurin-6-yl}pyrazol-1-yl]-1λ6-thiolane-1,1-dione

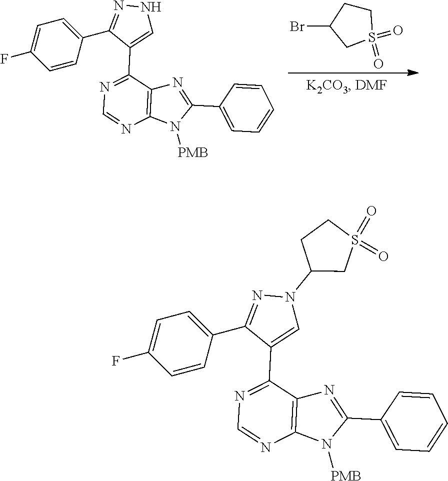

To a stirred mixture of 6-[3-(4-fluorophenyl)-1H-pyrazol-4-yl]-9-[(4-methoxyphenyl)methyl]-8-phenylpurine (Intermediate R, 195 mg, 0.409 mmol, 1 equiv.) and K₂CO₃ (113.11 mg, 0.818 mmol, 2 equiv.) in DMF (5 mL) at room temperature under nitrogen was added 3-bromo-1λ6-thiolane-1,1-dione (97.75 mg, 0.491 mmol, 1.2 equiv.) dropwise. The resulting mixture was stirred for 24 h at room temperature under nitrogen atmosphere then quenched by the addition of water (10 mL). The mixture was extracted with EtOAc (3×10 mL). The combined organic layers were washed with brine (2×5 mL) and dried over anhydrous Na₂SO₄. After filtration, the filtrate was concentrated under reduced pressure. The residue was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 32% B to 42% B in 8 min, 42% B; Wavelength: 254 nm; RT1 (min): 7) to afford the title compound (39 mg, 16.03%) as an off-white solid and afford 3-[5-(4-fluorophenyl)-4-{9-[(4-methoxyphenyl)methyl]-8-phenylpurin-6-yl}pyrazol-1-yl]-1,6-thiolane-1,1-dione (97 mg, 40%) as an off-white solid. m/z (ESI, +ve ion)=595.20 [M+H]⁺.

### Step 2. 3-[3-(4-fluorophenyl)-4-(8-phenyl-9H-purin-6-yl)-1H-pyrazol-1-yl]-1λ6-thiolane-1,1-dione

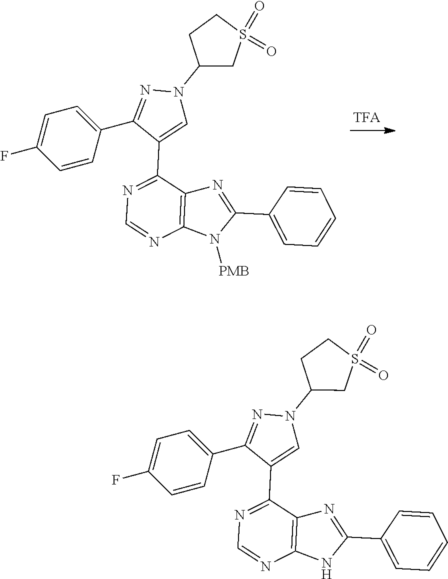

To an 8 mL round-bottom flask was added 3-[3-(4-fluorophenyl)-4-{9-[(4-methoxyphenyl)methyl]-8-phenylpurin-6-yl}pyrazol-1-yl]-1,6-thiolane-1,1-dione (22 mg, 0.037 mmol, 1 equiv.) and TFA (1 mL). The resulting mixture was stirred for 5 h at room temperature under nitrogen, then concentrated under reduced pressure. The residue was purified by Prep-HPLC with the following conditions (Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 40% B to 50% B in 8 min, 50% B; Wavelength: 254 nm; RT1 (min): 6.47) to afford Example 171 (2.8 mg, 16%) as a white solid. m/z (ESI, +ve ion)=475.10 [M+H]⁺. ¹H NMR (400 MHz, DMSO-d₆) δ 13.95 (s, 1H), 9.17 (s, 1H), 8.67 (s, 1H), 8.34-8.27 (m, 2H), 7.74 (s, 2H), 7.60 (m, 3H), 7.21 (t, J=8.8 Hz, 2H), 5.62-5.53 (m, 1H), 3.87 (m, 1H), 3.70-3.51 (m, 2H), 3.35 (s, 1H), 2.75 (m, 2H).

### Example 173. 3-{3-(4-fluorophenyl)-4-[6-(pyridin-2-yl)furo[2,3-d]pyrimidin-4-yl]-1H-pyrazol-1-yl}-1λ6-thietane-1,1-dione

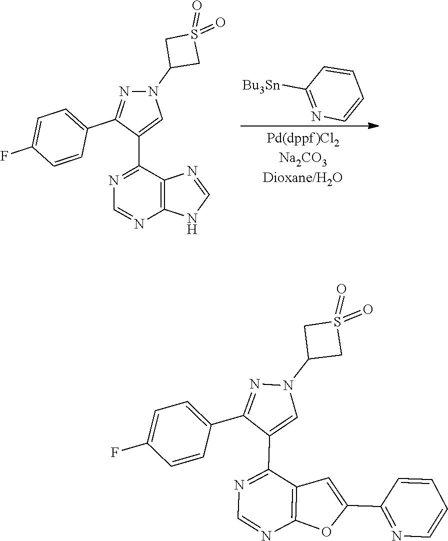

To a stirred solution of 3-(4-{6-bromofuro[2,3-d]pyrimidin-4-yl}-3-(4-fluorophenyl)pyrazol-1-yl)-1λ6-thietane-1,1-dione (Intermediate S, 200 mg, 0.432 mmol, 1 equiv.) and 2-(tributylstannyl)pyridine (476.80 mg, 1.296 mmol, 3 equiv.) in 1,4-dioxane (2 mL) and H₂O (0.5 mL) at room temperature under nitrogen was added Na₂CO₃ (91.51 mg, 0.864 mmol, 2 equiv.) and Pd(dppf)Cl₂·CH₂Cl₂ (35.17 mg, 0.043 mmol, 0.1 equiv.). The resulting mixture was stirred overnight at 80° C. The mixture was cooled to room temperature and quenched with water (12 mL). The mixture was extracted with CHCl₃ (3×15 mL). The combined organic layers were washed with brine (2×5 mL) and dried over anhydrous Na₂SO₄. After filtration, the filtrate was concentrated under reduced pressure. The crude product (120 mg) was purified by prep-HPLC with the following conditions (Column: Xselect CSH F-Phenyl OBD column, 19×250 mm, 5 μm; Mobile Phase A: water (0.1% FA), Mobile Phase B: ACN; Flow rate: 25 mL/min; Gradient: 39% B to 49% B in 11 min, 49% B; Wavelength: 254 nm; RT1 (min): 9.7) to afford Example 173 (26.8 mg, 13%) as a white solid. m/z (ESI+ve ion)=462.10 [M+H]⁺. ¹H NMR (400 MHz, Chloroform-d) δ 8.96 (s, 1H), 8.68-8.66 (m, 1H), 8.23 (s, 1H), 7.96-7.94 (m, 1H), 7.88-7.84 (m, 1H), 7.63-7.59 (m, 2H), 7.37-7.33 (m, 1H), 7.10-7.06 (m, 2H), 7.00 (s, 1H), 5.32-5.28 (m, 1H), 5.00-4.95 (m, 2H), 4.75-4.69 (m, 2H).

### Example 177. 3-[4-{6-[1-(cyclopropanesulfonyl)-1,2,3,6-tetrahydropyridin-4-yl]furo[2,3-d]pyrimidin-4-yl}-3-(4-fluorophenyl)-1H-pyrazol-1-yl]-1λ6-thietane-1,1-dione

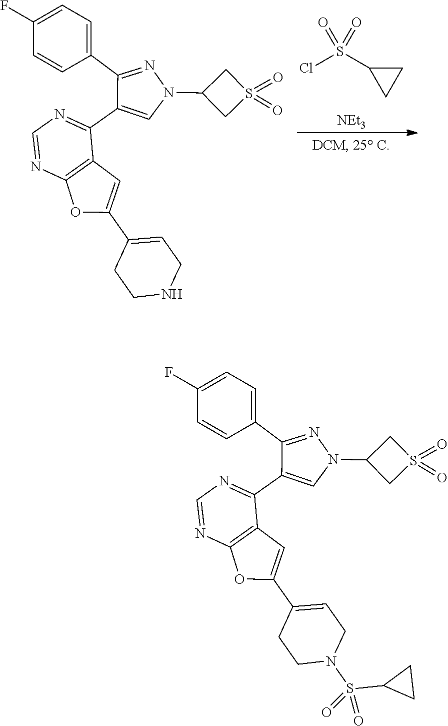

Cyclopropanesulfonyl chloride (45.3 mg, 322 μmol, 1.5 equiv.) was added to a mixture of 3-(3-(4-fluorophenyl)-4-(6-(1,2,3,6-tetrahydropyridin-4-yl)furo[2,3-d]pyrimidin-4-yl)-1H-pyrazol-1-yl)thietane 1,1-dioxide (Intermediate V, 100 mg, 215 μmol, 1.0 equiv.) and triethylamine (60.2 μL, 430 μmol, 2.0 equiv.) in DCM (4.77 mL) at room temperature, and the mixture was stirred at room temperature for 2 hours. The mixture was diluted with water (20 mL) and the aqueous layer was extracted with DCM (3×10 mL). The combined organic layers were washed with brine, dried over Na₂SO₄, filtered, and concentrated under reduced pressure. The crude material was purified by reverse phase chromatography (C18 column, MeCN in water with 10 mM ammonium formate, 10-100%, a gradient elution) to afford the title compound (21.7 mg, 18%) as a solid. ¹H NMR (400 MHz, DMSO-d₆) δ 8.75 (s, 1H), 8.68 (s, 1H), 7.59-7.50 (m, 2H), 7.21-7.14 (m, 2H), 6.65 (s, 1H), 6.60 (s, 1H), 5.47 (dq, J=8.2, 6.2 Hz, 1H), 4.89-4.81 (m, 4H), 4.01 (d, J=2.4 Hz, 2H), 3.44 (t, J=5.7 Hz, 2H), 2.69-2.59 (m, 1H), 2.44-2.40 (m, 2H), 0.98-0.90 (m, 4H). m/z (ESI, +ve ion)=566.4 [M+H]⁺.

### Example 178. 3-{4-[6-(3-cyclopropylphenyl)furo[2,3-d]pyrimidin-4-yl]-3-(4-fluorophenyl)-1H-pyrazol-1-yl}-16-thietane-1,1-dione

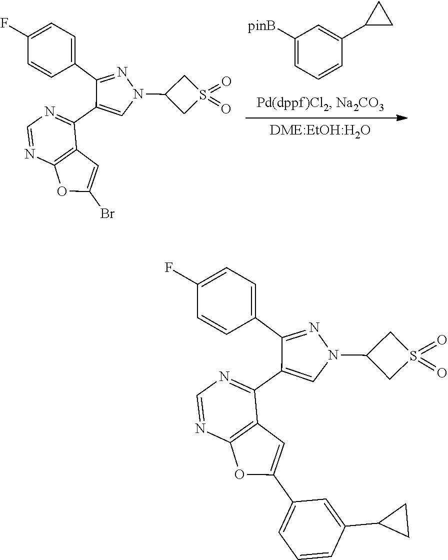

1,1′-Bis(diphenylphosphino)ferrocene dichloropalladium (II) (11.8 mg, 16.2 μmol, 0.1 equiv.) was added to a degassed mixture of 3-(4-{6-bromofuro[2,3-d]pyrimidin-4-yl}-3-(4-fluorophenyl)pyrazol-1-yl)-1,6-thietane-1,1-dione (Intermediate S, 75.0 mg, 162 μmol, 1.0 equiv), 3-cyclopropyl-benzeneboronic acid (31.5 mg, 194 μmol, 1.2 equiv.) and sodium carbonate (52.0 mg, 486 μmol, 3.0 equiv.) in a mixture of DME (2.25 mL), EtOH (750 μL) and H₂O (300 μL). The mixture was stirred for 45 minutes at 110° C. in a microwave reactor, then diluted with EtOAc (5 mL). The mixture was filtered and concentrated under reduced pressure. The crude material was purified by column chromatography (EtOAc in hexanes, 10-100%, a gradient elution), followed by reverse phase chromatography (C18 column, MeCN in water with 10 mM ammonium formate, 20-100%, a gradient elution) to afford the title compound (42.6 mg, 53%) as a solid. ¹H NMR (400 MHz, DMSO-d₆) δ 8.81 (s, 1H), 8.78 (s, 1H), 7.68-7.60 (m, 3H), 7.56 (app s, 1H), 7.43 (app t, J=7.7 Hz, 1H), 7.32 (s, 1H), 7.25-7.17 (m, 3H), 5.57-5.48 (m, 1H), 4.97-4.86 (m, 4H), 2.07-1.99 (m, 1H), 1.06-1.00 (m, 2H), 0.80-0.74 (m, 2H). m/z (ESI, +ve ion)=501.3 [M+H]⁺.

### Example 179. 4-[1-ethyl-3-(4-fluorophenyl)-1H-pyrazol-4-yl]-6-phenylfuro[2,3-d]pyrimidine

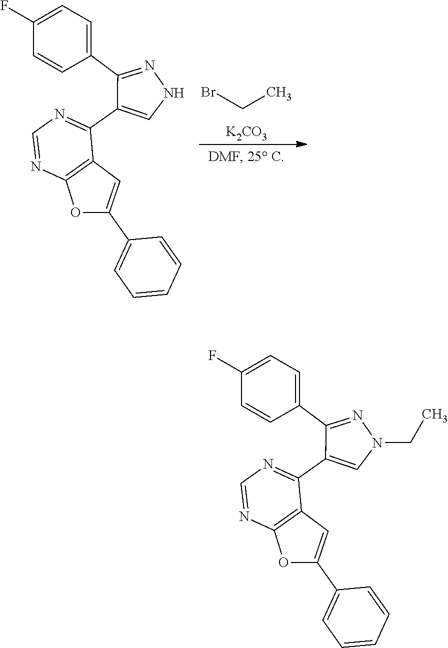

4-[3-(4-Fluorophenyl)-1H-pyrazol-4-yl]-6-phenylfuro[2,3-d]pyrimidine (Intermediate N, 100 mg, 281 μmol, 1.0 equiv.) was dissolved in DMF (935 μL).Sodium hydride (8.08 mg, 337 μmol, 1.2 equiv., 60% in dispersion in mineral oil) was added, followed by bromoethane (25.4 μL, 337 μmol, 1.2 equiv.). The mixture was stirred at room temperature for 16 hours. The mixture was diluted with water and the aqueous layer was extracted with EtOAc (3×10 mL). The combined organic layers were washed with water (3×10 mL) then with brine (10 mL) and concentrated under reduced pressure. The crude material was purified by column chromatography (EtOAc in hexanes, 0-100%, a gradient elution) to afford the title compound (35.4 mg, 33%) as a solid. ¹H NMR (400 MHz, DMSO-d₆) δ 8.76 (s, 1H), 8.63 (s, 1H), 7.95-7.88 (m, 2H), 7.63-7.53 (m, 4H), 7.53-7.46 (m, 1H), 7.44 (s, 1H), 7.23-7.15 (m, 2H), 4.31 (q, J=7.3 Hz, 2H), 1.53 (t, J=7.3 Hz, 3H). m/z (ESI, +ve ion)=385.2 [M+H]⁺.

### Example 181. 3-[3-(4-fluorophenyl)-4-{6-[1-(methanesulfonyl)-1,2,3,6-tetrahydropyridin-4-yl]furo[2,3-d]pyrimidin-4-yl}-1H-pyrazol-1-yl]-1λ6-thietane-1,1-dione

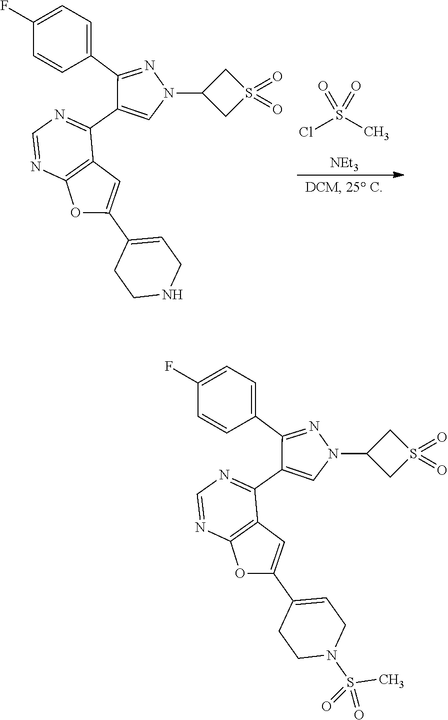

Methanesulfonyl chloride (16.7 μL, 215 μmol, 1.0 equiv.) was added to a mixture of 3-(3-(4-fluorophenyl)-4-(6-(1,2,3,6-tetrahydropyridin-4-yl)furo[2,3-d]pyrimidin-4-yl)-1H-pyrazol-1-yl)thietane 1,1-dioxide (Intermediate V, 100 mg, 215 μmol, 1.0 equiv.) and triethylamine (60.2 μL, 430 μmol, 2.0 equiv.) in DCM (4.77 mL) at room temperature. The reaction mixture was stirred at room temperature for 2 hours. The solvent was evaporated, and the crude material was purified by prep-HPLC (Buchi Pure C850, Phenomenex Luna C18, 10 μm, 250 mm×21.2 mm, eluent: MeOH in water with 10 mM ammonium bicarbonate, 35-100%, a gradient elution) to afford the title compound (29.6 mg, 25%) as a solid. ¹H NMR (400 MHz, DMSO-d₆) δ 8.79 (s, 1H), 8.72 (s, 1H), 7.63-7.55 (m, 2H), 7.25-7.17 (m, 2H), 6.72 (s, 1H), 6.64 (s, 1H), 5.51 (dq, J=8.2, 6.1 Hz, 1H), 4.94-4.82 (m, 4H), 3.99-3.94 (m, 2H), 3.40 (t, J=5.6 Hz, 2H), 2.96 (s, 3H). 2H hidden under DMSO peak. m/z (ESI, +ve ion)=544.3 [M+H]⁺.

### Example 184. cis-Diastereomer obtained as a racemate of 4-[1-(3-fluorooxan-4-yl)-3-(4-fluorophenyl)-1H-pyrazol-4-yl]-6-phenylfuro[2,3-d]pyrimidine

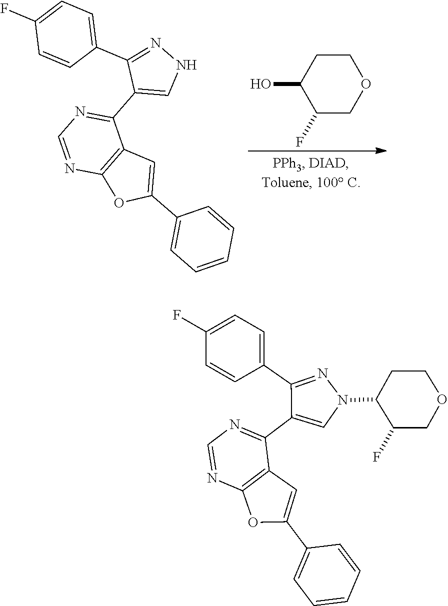

To a mixture of (trans)-3-fluorooxan-4-ol (21.6 mg, 0.180 mmol, 1 equiv.), 4-[3-(4-Fluorophenyl)-1H-pyrazol-4-yl]-6-phenylfuro[2,3-d]pyrimidine (Intermediate N, 53.5 mg, 0.150 mmol, 0.83 equiv.) and Ph₃P (59 mg, 0.225 mmol, 1.25 equiv.) was added toluene (2.5 mL) under nitrogen atmosphere. The reaction mixture was cooled to 0° C. then DIAD (59 μL, 0.298 mmol, 1.66 equiv.) was added dropwise at 0° C. The reaction was stirred at 0° C. for 30 minutes and heated to 100° C. The reaction was stirred at 100° C. overnight. After cooling to room temperature, the solvent was removed under reduced pressure. The residue was purified by silica gel column chromatography and eluted with 0-100% of EtOAc in PE to give 30 mg of crude product (˜80% purity) as a light yellow solid. The crude product was further purified by prep-HPLC with the following condition: (Column: Xselect CSH C18 OBD Column 30×150 mm 5 μm; Mobile Phase A: water (10 mmol/L NH₄HCO₃), Mobile Phase B: ACN; Flow rate: 60 mL/min; Gradient: 55% B to 61% B in 8 min, 61% B; Wavelength: 254 nm; RT1 (min): 6.4 to afford Example 184 (12.8 mg, 16%) as a white solid. m/z (ESI, +ve ion)=459.10 [M+H]⁺. ¹H NMR (400 MHz, Chloroform-d) δ 8.93 (s, 1H), 8.24 (d, J=1.6 Hz, 1H), 7.68-7.66 (m, 2H), 7.89-7.50 (m, 2H), 7.48-7.39 (m, 3H), 7.12-7.08 (m, 2H), 6.18 (s, 1H), 5.07 (d, J=48.8 Hz, 1H), 4.78-4.66 (m, 1H), 4.37-4.26 (m, 2H), 3.78-3.65 (m, 2H), 2.69-2.64 (m, 1H), 2.18 (dd, J=12.6, 2.6 Hz, 1H).

### Example 189. 4-{3-(4-fluorophenyl)-1-[(3R,5R)-1-(methanesulfonyl)-5-methylpyrrolidin-3-yl]-1H-pyrazol-4-yl}-6-phenylfuro[2,3-d]pyrimidine

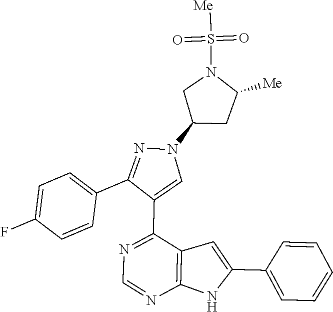 [SMILES: C[C@@H]1C[C@@H](n2cc(-c3ncnc4[nH]c(-c5ccccc5)cc34)c(-c3ccc(F)cc3)n2)CN1S(C)(=O)=O]

### Step 1. tert-butyl (2R,4S)-2-methyl-4-(tosyloxy)pyrrolidine-1-carboxylate

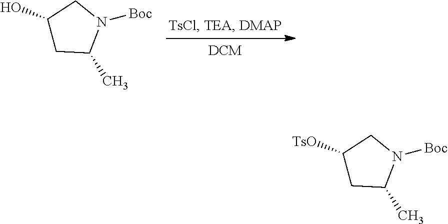 [SMILES: C[C@@H]1C[C@H](O)CN1C(=O)OC(C)(C)C ; Cc1ccc(S(=O)(=O)O[C@H]2C[C@@H](C)N(C(=O)OC(C)(C)C)C2)cc1]

To a stirred solution of tert-butyl (2R,4S)-4-hydroxy-2-methylpyrrolidine-1-carboxylate (300 mg, 1.491 mmol, 1 equiv.) in DCM (3 mL) at 20° C. under nitrogen atmosphere was added TEA (0.41 mL, 2.982 mmol, 2 equiv.) and DMAP (18.21 mg, 0.149 mmol, 0.1 equiv.). To this mixture at 0° C. was added TsCl (340.99 mg, 1.789 mmol, 1.2 equiv.). The reaction was stirred for an additional 16 hours at 20° C. and then quenched with water (20 mL) and extracted with DCM (3×25 mL). The combined organic layers were washed with brine (3×25 mL) and dried over anhydrous Na₂SO₄. After filtration, the filtrate was concentrated under reduced pressure to get the residue. The residue was purified by silica gel column chromatography and eluted with 8% EtOAc in PE to afford the title compound (430 mg, 81%) as white solid. m/z (ESI, +ve ion)=373.10 [M+NH₄].

### Step 2. tert-butyl (2R,4R)-4-(3-(4-fluorophenyl)-4-(6-phenylfuro[2,3-d]pyrimidin-4-yl)-1H-pyrazol-1-yl)-2-methylpyrrolidine-1-carboxylate

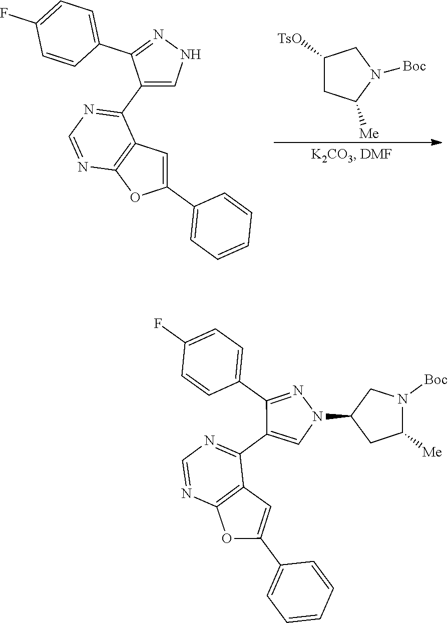 [SMILES: Fc1ccc(-c2n[nH]cc2-c2ncnc3oc(-c4ccccc4)cc23)cc1 ; Cc1ccc(S(=O)(=O)O[C@H]2C[C@@H](C)N(C(=O)OC(C)(C)C)C2)cc1 ; C[C@@H]1C[C@@H](n2cc(-c3ncnc4oc(-c5ccccc5)cc34)c(-c3ccc(F)cc3)n2)CN1C(=O)OC(C)(C)C]

To a stirred solution of 4-[3-(4-Fluorophenyl)-1H-pyrazol-4-yl]-6-phenylfuro[2,3-d]pyrimidine (Intermediate N, 100 mg, 0.281 mmol, 1.00 equiv.) and K₂CO₃ (77.56 mg, 0.562 mmol, 2 equiv.) in DMF (1 mL) at 20° C. under nitrogen was added tert-butyl (2R,4S)-2-methyl-4-[(4-methylbenzenesulfonyl)oxy]pyrrolidine-1-carboxylate (119.69 mg, 0.337 mmol, 1.2 equiv.). The resulting mixture was stirred for 16 hours at 60° C. under nitrogen atmosphere and then cooled to 20° C. The reaction mixture was quenched by the addition of water (5 mL) at 20° C. and extracted with EtOAc (3×10 mL). The combined organic layers were washed with brine (3×10 mL) and dried over anhydrous Na₂SO₄. After filtration, the filtrate was concentrated under reduced pressure. The residue was purified by Prep-TLC (EtOAc/PE=1/1) to afford the title compound (93 mg, 61%) as white solid. m/z (ESI, +ve ion)=540.20 [M+H]⁺.

### Step 3. 4-(3-(4-fluorophenyl)-1-((3R,5R)-5-methylpyrrolidin-3-yl)-1H-pyrazol-4-yl)-6-phenylfuro[2,3-d]pyrimidine

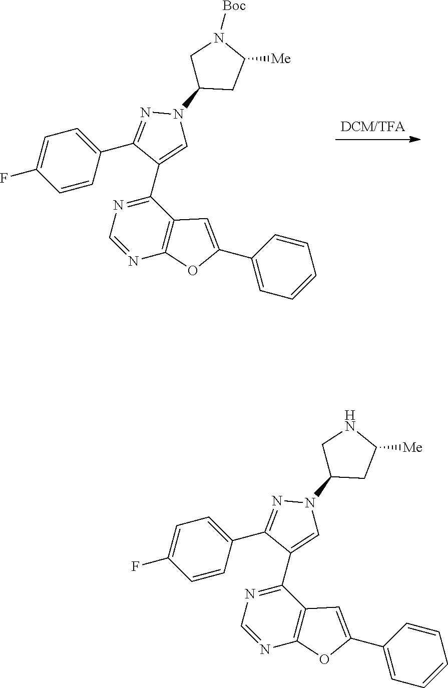 [SMILES: C[C@@H]1C[C@@H](n2cc(-c3ncnc4oc(-c5ccccc5)cc34)c(-c3ccc(F)cc3)n2)CN1C(=O)OC(C)(C)C ; C[C@@H]1C[C@@H](n2cc(-c3ncnc4oc(-c5ccccc5)cc34)c(-c3ccc(F)cc3)n2)CN1]

To a stirred solution of tert-butyl (2R,4R)-4-[3-(4-fluorophenyl)-4-{6-phenylfuro[2,3-d]pyrimidin-4-yl}pyrazol-1-yl]-2-methylpyrrolidine-1-carboxylate (93 mg, 0.172 mmol, 1 equiv.) in DCM (3 mL) was added TFA (1 mL) under nitrogen atmosphere. The resulting mixture was stirred for 30 min at 20° C. and then concentrated under reduced pressure. This resulted in the title compound TFA salt (75.74 mg, 99.99%) as a yellow oil. m/z (ESI, +ve ion)=440.00 [M+H]+

### Step 4. 4-{3-(4-fluorophenyl)-1-[(3R,5R)-1-(methanesulfonyl)-5-methylpyrrolidin-3-yl]-1H-pyrazol-4-yl}-6-phenylfuro[2,3-d]pyrimidine

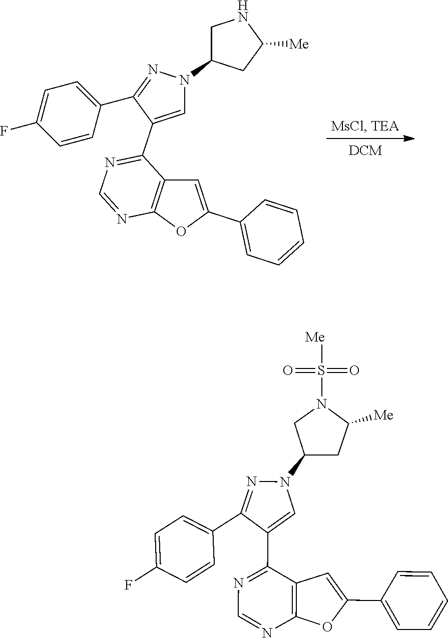 [SMILES: C[C@@H]1C[C@@H](n2cc(-c3ncnc4oc(-c5ccccc5)cc34)c(-c3ccc(F)cc3)n2)CN1 ; C[C@@H]1C[C@@H](n2cc(-c3ncnc4oc(-c5ccccc5)cc34)c(-c3ccc(F)cc3)n2)CN1S(C)(=O)=O]

To a stirred solution of 3-(4-fluorophenyl)-1-[(3R,5R)-5-methylpyrrolidin-3-yl]-4-{6-phenylfuro[2,3-d]pyrimidin-4-yl}pyrazole (93 mg, 0.212 mmol, 1 equiv.) and TEA (0.06 mL, 0.424 mmol, 2 equiv.) in DCM (1 mL) at 0° C. under nitrogen was added MsCl (29 mg, 0.254 mmol, 1.2 equiv.). The resulting mixture was stirred for 16 h at 30° C. and then quenched by the addition of water (5 mL) at 20° C. The resulting mixture was extracted with DCM (3×5 mL). The combined organic layers were washed with brine (3×5 mL) and dried over anhydrous Na₂SO₄. After filtration, the filtrate was concentrated under reduced pressure. The residue was purified by Prep-HPLC with the following conditions: Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 53% B to 63% B in 8 min, 63% B; Wavelength: 254 nm; RT1 (min): 7 to afford the title compound (39.9 mg, 35%) as a white solid. m/z (ESI, +ve ion)=518.20 [M+H]⁺. ¹H-NMR (400 MHz, Chloroform-d) δ 8.92 (s, 1H), 8.29 (s, 1H), 7.69-7.67 (d, J=8 Hz, 2H), 7.55-7.52 (m, 2H), 7.48-7.40 (m, 3H), 7.12-7.02 (m, 2H), 6.25 (s, 1H), 5.07-5.02 (m, 1H), 4.50-4.25 (m, 1H), 4.12-4.08 (m, 1H), 4.04-3.99 (m, 1H), 3.01-2.83 (m, 4H), 2.99-2.22 (m, 1H), 1.58 (d, J=6.8 Hz, 3H).

### Example 190. 3-[3-(4-fluorophenyl)-4-(6-{3-[(morpholin-4-yl)methyl]phenyl}furo[2,3-d]pyrimidin-4-yl)-1H-pyrazol-1-yl]-16-thietane-1,1-dione

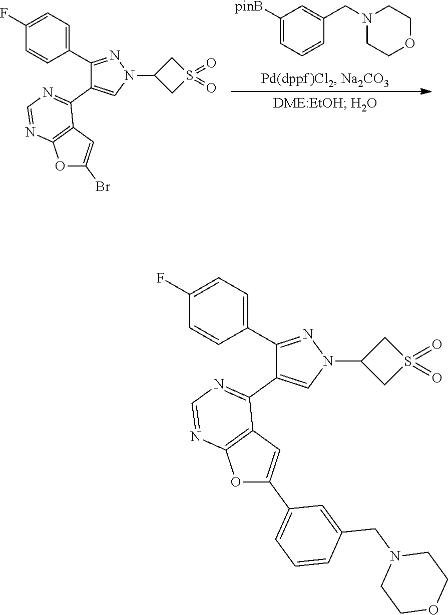

1,1′-Bis(diphenylphosphino)ferrocene dichloropalladium (II) (12.5 mg, 16.2 μmol, 0.1 equiv.) was added to a degassed mixture of 3-(4-{6-bromofuro[2,3-d]pyrimidin-4-yl}-3-(4-fluorophenyl)pyrazol-1-yl)-1,6-thietane-1,1-dione (Intermediate S, 75.0 mg, 162 μmol, 1.0 equiv.), 3-(4-morpholinomethyl)phenylboronic acid pinacol ester (62.6 mg, 202 μmol, 1.25 equiv.) and sodium carbonate (52.0 mg, 486 μmol, 3.0 equiv.) in a mixture of DME (2.25 mL), EtOH (750 μL) and H₂O (300 μL). The mixture was stirred for 45 minutes at 110° C. in a microwave reactor then diluted with EtOAc (5 mL). The mixture was filtered and concentrated under reduced pressure. The crude material was purified by reverse phase chromatography (C18 column, MeCN in water with 10 mM ammonium formate, 10-40%, a gradient elution) to afford the title compound (42.3 mg, 47%) as a solid. ¹H NMR (400 MHz, DMSO-d₆) δ 8.81 (s, 1H), 8.79 (s, 1H), 7.81 (s, 1H), 7.79 (d, 1H), 7.66-7.62 (m, 2H), 7.52 (t, J=7.6 Hz, 1H), 7.44 (d, J=7.6 Hz, 1H), 7.34 (s, 1H), 7.22 (m, 2H), 5.57-5.48 (m, 1H), 4.99-4.84 (m, 4H), 3.64-3.57 (m, 4H), 3.55 (s, 2H), 2.44-2.35 (m, 4H). m/z (ESI, +ve ion)=560.3 [M+H]⁺.

### Example 197. 4-{3-(4-fluorophenyl)-1-[(3S,5S)-1-(methanesulfonyl)-5-methylpyrrolidin-3-yl]-1H-pyrazol-4-yl}-6-phenylfuro[2,3-d]pyrimidine

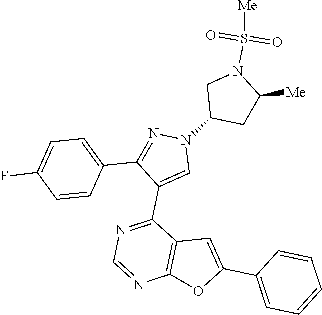 [SMILES: C[C@H]1C[C@H](n2cc(-c3ncnc4oc(-c5ccccc5)cc34)c(-c3ccc(F)cc3)n2)CN1S(C)(=O)=O]

### Step 1. tert-butyl (2S,4R)-2-methyl-4-[(4-methylbenzenesulfonyl)oxy]pyrrolidine-1-carboxylate

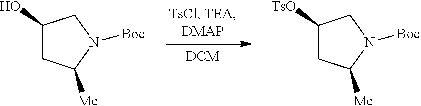 [SMILES: C[C@H]1C[C@@H](O)CN1C(=O)OC(C)(C)C ; Cc1ccc(S(=O)(=O)O[C@@H]2C[C@H](C)N(C(=O)OC(C)(C)C)C2)cc1]

To a stirred solution of tert-butyl (2S,4R)-4-hydroxy-2-methylpyrrolidine-1-carboxylate (400 mg, 1.987 mmol, 1 equiv.) and DMAP (24.28 mg, 0.199 mmol, 0.1 equiv.) in DCM (10 mL) at 0° C. under nitrogen were added TEA (301.67 mg, 2.981 mmol, 1.5 equiv.) and TsCl (454.66 mg, 2.384 mmol, 1.2 equiv.) dropwise. The resulting mixture was stirred for 2 h at room temperature under nitrogen atmosphere then quenched by the addition of sat. NH₄Cl (aq., 20 mL) at 0° C. The resulting mixture was diluted with water (30 mL) and extracted with DCM (3×50 mL). The combined organic layers were washed with brine (2×50 mL) and dried over anhydrous MgSO₄. After filtration, the filtrate was concentrated under reduced pressure. The residue was purified by Prep-TLC (PE/EtOAc 1/1) to afford the title compound (423 mg, 60%) as a colorless oil. No MS on LCMS. ¹H NMR (400 MHz, Chloroform-d) δ 7.80 (d, J=8.2 Hz, 2H), 7.36 (d, J=8.0 Hz, 2H), 5.02-5.00 (m, 1H), 3.94 (s, 1H), 3.61-3.59 (m, 1H), 3.49 (s, 1H), 2.46 (s, 3H), 2.23-2.20 (m, 1H), 1.92-1.88 (m, 1H), 1.44 (s, 9H), 1.27 (d, J=6.5 Hz, 3H)

### Step 2. tert-butyl (2S,4S)-4-[3-(4-fluorophenyl)-4-{6-phenylfuro[2,3-d]pyrimidin-4-yl}pyrazol-1-yl]-2-methylpyrrolidine-1-carboxylate

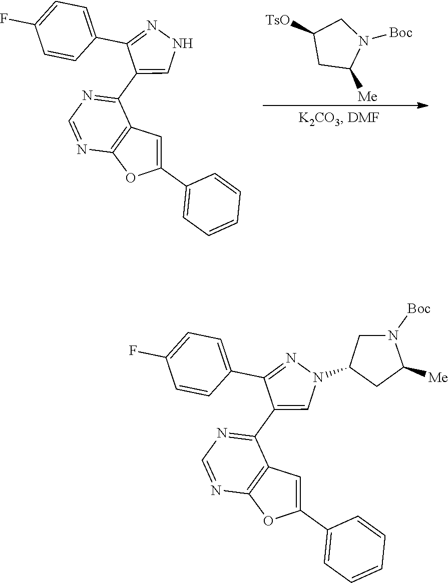 [SMILES: Fc1ccc(-c2n[nH]cc2-c2ncnc3oc(-c4ccccc4)cc23)cc1 ; Cc1ccc(S(=O)(=O)O[C@@H]2C[C@H](C)N(C(=O)OC(C)(C)C)C2)cc1 ; C[C@H]1C[C@H](n2cc(-c3ncnc4oc(-c5ccccc5)cc34)c(-c3ccc(F)cc3)n2)CN1C(=O)OC(C)(C)C]

To a stirred solution of 4-[3-(4-Fluorophenyl)-1H-pyrazol-4-yl]-6-phenylfuro[2,3-d]pyrimidine (Intermediate N, 150 mg, 0.421 mmol, 1.00 equiv.) in DMF (6 mL) at room temperature under nitrogen atmosphere was added K₂CO₃ (116.35 mg, 0.842 mmol, 2 equiv.). The resulting mixture was stirred for 15 min at room temperature. To the mixture at room temperature was added tert-butyl (2S,4R)-2-methyl-4-[(4-methylbenzenesulfonyl)oxy]pyrrolidine-1-carboxylate (179.54 mg, 0.505 mmol, 1.2 equiv.) dropwise. The resulting mixture was stirred for additional 16 h at 100° C., cooled to room temperature, and quenched by the addition of water (30 mL) at 0° C. The resulting mixture was extracted with DCM (3×30 mL). The combined organic layers were washed with brine (3×30 mL) and dried over anhydrous MgSO₄. After filtration, the filtrate was concentrated under reduced pressure. The residue was purified by Prep-TLC (PE/EtOAc 2/1) to afford the title compound (160 mg, 70%) as a yellow oil. m/z (ESI, +ve ion)=540.25 [M+H]⁺.

### Step 3. 3-(4-fluorophenyl)-1-[(3S,5S)-5-methylpyrrolidin-3-yl]-4-{6-phenylfuro[2,3-d]pyrimidin-4-yl}pyrazole

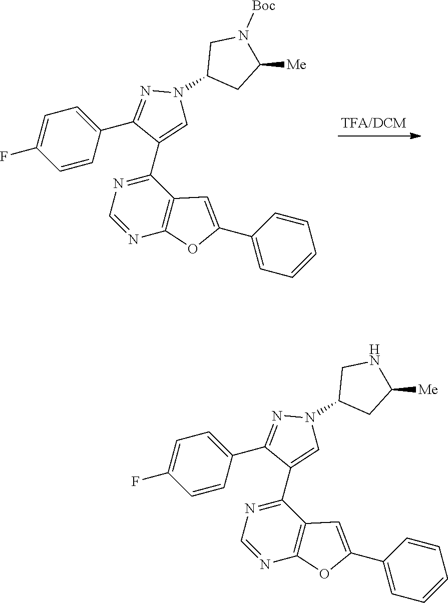 [SMILES: C[C@H]1C[C@H](n2cc(-c3ncnc4oc(-c5ccccc5)cc34)c(-c3ccc(F)cc3)n2)CN1C(=O)OC(C)(C)C ; C[C@H]1C[C@H](n2cc(-c3ncnc4oc(-c5ccccc5)cc34)c(-c3ccc(F)cc3)n2)CN1]

To a stirred solution of tert-butyl (2S,4S)-4-[3-(4-fluorophenyl)-4-{6-phenylfuro[2,3-d]pyrimidin-4-yl}pyrazol-1-yl]-2-methylpyrrolidine-1-carboxylate (165 mg, 0.306 mmol, 1 equiv.) in DCM (3 mL) at room temperature under nitrogen was added TFA (1 mL, 1.308 mmol) dropwise. The resulting mixture was stirred for 1 h at room temperature under nitrogen atmosphere then quenched by the addition of sat. NaHCO₃ (aq., 10 mL) at 0° C. The resulting mixture was diluted with water (20 mL) and extracted with EtOAc (3×30 mL). The combined organic layers were washed with brine (3×30 mL) and dried over anhydrous Na₂SO₄. After filtration, the filtrate was concentrated under reduced pressure. The residue was purified by Prep-TLC (PE/EtOAc 1/1) to afford the title compound (115 mg, 86%) as a yellow oil. m/z (ESI, +ve ion)=440.20 [M+H]+

### Step 4. 4-{3-(4-fluorophenyl)-1-[(3S,5S)-1-(methanesulfonyl)-5-methylpyrrolidin-3-yl]-1H-pyrazol-4-yl}-6-phenylfuro[2,3-d]pyrimidine

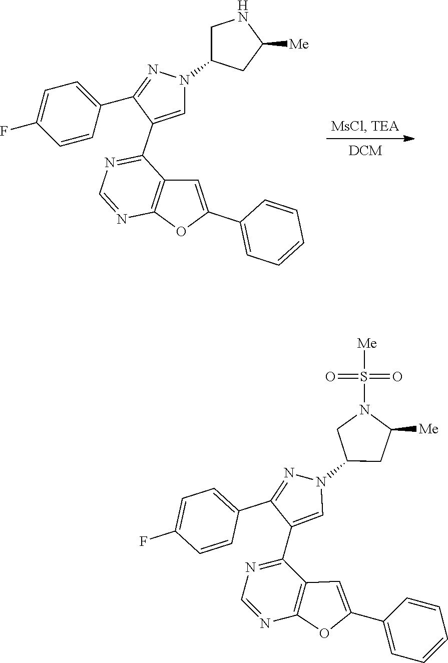 [SMILES: C[C@H]1C[C@H](n2cc(-c3ncnc4oc(-c5ccccc5)cc34)c(-c3ccc(F)cc3)n2)CN1 ; C[C@H]1C[C@H](n2cc(-c3ncnc4oc(-c5ccccc5)cc34)c(-c3ccc(F)cc3)n2)CN1S(C)(=O)=O]

To a stirred solution of 3-(4-fluorophenyl)-1-[(3S,5S)-5-methylpyrrolidin-3-yl]-4-{6-phenylfuro[2,3-d]pyrimidin-4-yl}pyrazole (110 mg, 0.250 mmol, 1 equiv.) in DCM (3.3 mL) at 0° C. under nitrogen was added TEA (76 mg, 0.750 mmol, 3 equiv.) dropwise. The resulting mixture was stirred for 20 min at 0° C. under nitrogen atmosphere. To this mixture at 0° C. was added MsCl (34.40 mg, 0.300 mmol, 1.2 equiv.) dropwise. The resulting mixture was stirred for an additional 1 h at 0° C., then quenched by the addition of sat. NH₄Cl (aq., 5 mL) at 0° C. The resulting mixture was diluted with water (25 mL) and extracted with EtOAc (3×30 mL). The combined organic layers were washed with brine (3×30 mL) and dried over anhydrous Na₂SO₄. After filtration, the filtrate was concentrated under reduced pressure. The crude product was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 53% B to 63% B in 8 min, 63% B; Wavelength: 254 nm; RT1 (min): 7) to afford Example 197 (44 mg, 34%) as a white solid. m/z (ESI+ve ion)=518.10 [M+H]⁺. ¹H-NMR (400 MHz, Chloroform-d) δ 9.01 (s, 1H), 8.63 (s, 1H), 7.66-7.63 (m, 2H), 7.55-7.46 (m, 5H), 7.14 (t, J=8.4 Hz, 2H), 6.09 (s, 1H), 5.08 (s, 1H), 4.33-4.26 (m, 1H), 4.12-3.99 (m, 2H), 2.91 (s, 4H), 2.32-2.42 (m, 1H), 1.50 (d, J=6.0 Hz, 3H).

### Example 198. 4-{3-(4-fluorophenyl)-1-[(3R,5S)-1-(methanesulfonyl)-5-methylpyrrolidin-3-yl]-1H-pyrazol-4-yl}-6-phenylfuro[2,3-d]pyrimidine

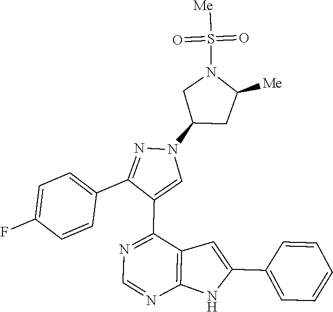 [SMILES: C[C@H]1C[C@@H](n2cc(-c3ncnc4[nH]c(-c5ccccc5)cc34)c(-c3ccc(F)cc3)n2)CN1S(C)(=O)=O]

### Step 1. tert-butyl (2S, 4S)-2-methyl-4-[(4-methylbenzenesulfonyl)oxy]pyrrolidine-1-carboxylate

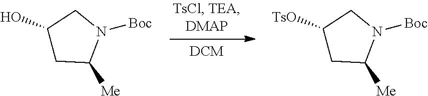 [SMILES: C[C@H]1C[C@H](O)CN1C(=O)OC(C)(C)C ; Cc1ccc(S(=O)(=O)O[C@H]2C[C@H](C)N(C(=O)OC(C)(C)C)C2)cc1]

To a stirred solution of tert-butyl (2S, 4S)-4-hydroxy-2-methylpyrrolidine-1-carboxylate (400 mg, 1.987 mmol, 1 equiv.) and DMAP (24.28 mg, 0.199 mmol, 0.1 equiv.) in DCM (10 mL) at 0° C. under nitrogen was added TEA (301.67 mg, 2.981 mmol, 1.5 equiv.) and TsCl (454.66 mg, 2.384 mmol, 1.2 equiv.). The resulting mixture was stirred for 2 h at room temperature under nitrogen atmosphere, then quenched by the addition of sat. NH₄Cl (aq., 20 mL) at 0° C. The resulting mixture was diluted with water (30 mL) and extracted with DCM (3×50 mL). The combined organic layers were washed with brine (2×50 mL) and dried over anhydrous MgSO₄. After filtration, the filtrate was concentrated under reduced pressure. The residue was purified by Prep-TLC (PE/EtOAc=1/1) to afford the title compound (465 mg, 66%) as a colorless oil. ¹H NMR (400 MHz, Chloroform-d) δ 7.81-7.75 (m, 2H), 7.35 (d, J=8.0 Hz, 2H), 4.98-4.96 (m, 1H), 3.98-3.91 (m, 1H), 3.63-3.59 (m, 1H), 3.42-3.38 (m, 1H), 2.45 (s, 3H), 2.29-2.26 (m, 1H), 1.77-1.69 (m, 1H), 1.43 (s, 9H), 1.21 (d, J=6.2 Hz, 3H).

### Step 2. tert-butyl (2S, 4R)-4-[3-(4-fluorophenyl)-4-{6-phenylfuro[2,3-d]pyrimidin-4-yl}pyrazol-1-yl]-2-methylpyrrolidine-1-carboxylate

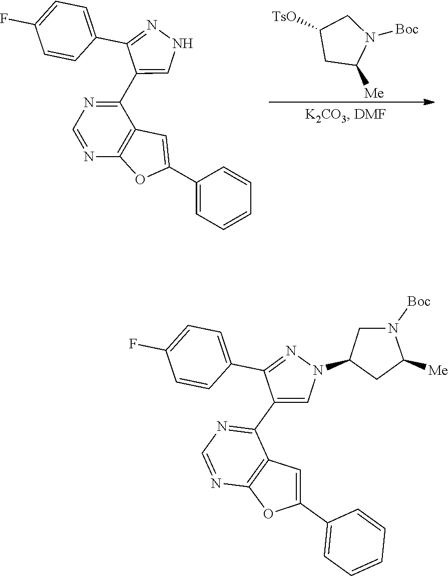 [SMILES: Fc1ccc(-c2n[nH]cc2-c2ncnc3oc(-c4ccccc4)cc23)cc1 ; Cc1ccc(S(=O)(=O)O[C@H]2C[C@H](C)N(C(=O)OC(C)(C)C)C2)cc1 ; C[C@H]1C[C@@H](n2cc(-c3ncnc4oc(-c5ccccc5)cc34)c(-c3ccc(F)cc3)n2)CN1C(=O)OC(C)(C)C]

To a stirred solution of 4-[3-(4-Fluorophenyl)-1H-pyrazol-4-yl]-6-phenylfuro[2,3-d]pyrimidine (Intermediate N, 150 mg, 0.421 mmol, 1.00 equiv.) in DMF (6 mL) at room temperature under nitrogen was added K₂CO₃ (116.35 mg, 0.842 mmol, 2 equiv.). The resulting mixture was stirred for 15 min at room temperature under nitrogen atmosphere. To this mixture was added tert-butyl (2S, 4S)-2-methyl-4-[(4-methylbenzenesulfonyl)oxy]pyrrolidine-1-carboxylate (149.62 mg, 0.421 mmol, 1 equiv.) at room temperature. The mixture was stirred for additional 16 h at 100° C. and then cooled to room temperature. The reaction was quenched by the addition of water (30 mL) at 0° C., then extracted with DCM (3×30 mL). The combined organic layers were washed with brine (3×30 mL) and dried over anhydrous MgSO₄. After filtration, the filtrate was concentrated under reduced pressure. The residue was purified by Prep-TLC (PE/EtOAc 2/1) to afford the title compound (115 mg, 51%) as a yellow oil. m/z (ESI, +ve ion)=540.15 [M+H]⁺.

### Step 3. 3-(4-fluorophenyl)-1-[(3R,5S)-5-methylpyrrolidin-3-yl]-4-{6-phenylfuro[2,3-d]pyrimidin-4-yl}pyrazole

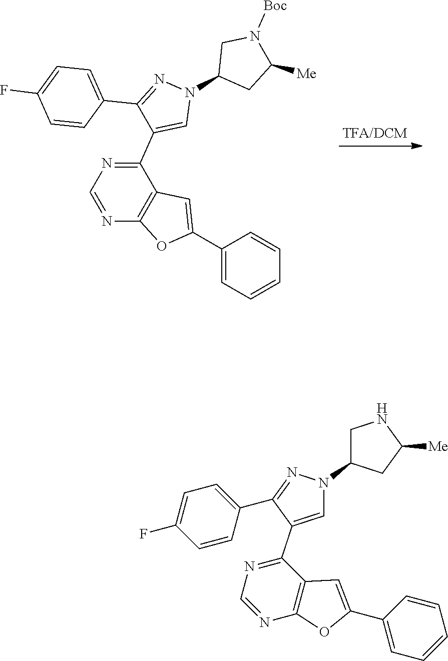 [SMILES: C[C@H]1C[C@@H](n2cc(-c3ncnc4oc(-c5ccccc5)cc34)c(-c3ccc(F)cc3)n2)CN1C(=O)OC(C)(C)C ; C[C@H]1C[C@@H](n2cc(-c3ncnc4oc(-c5ccccc5)cc34)c(-c3ccc(F)cc3)n2)CN1]

To a stirred solution of tert-butyl (2S,4R)-4-[3-(4-fluorophenyl)-4-{6-phenylfuro[2,3-d]pyrimidin-4-yl}pyrazol-1-yl]-2-methylpyrrolidine-1-carboxylate (110 mg, 0.204 mmol, 1 equiv.) in DCM (3 mL) at room temperature under nitrogen was added TFA (1 mL) dropwise. The resulting mixture was stirred for 1 h at room temperature under nitrogen. The reaction was quenched by the addition of sat. NaHCO₃ (aq., 10 mL) at 0° C. The resulting mixture was diluted with water (20 mL) and extracted with EtOAc (3×30 mL). The combined organic layers were washed with brine (3×30 mL) and dried over anhydrous Na₂SO₄. After filtration, the filtrate was concentrated under reduced pressure. The residue was purified by Prep-TLC (PE/EtOAc=1/1) to afford the title compound (89 mg, 99%) as a yellow oil. m/z (ESI, +ve ion)=440.15 [M+H]⁺

### Step 4. 4-{3-(4-fluorophenyl)-1-[(3R,5S)-1-(methanesulfonyl)-5-methylpyrrolidin-3-yl]-1H-pyrazol-4-yl}-6-phenylfuro[2,3-d]pyrimidine

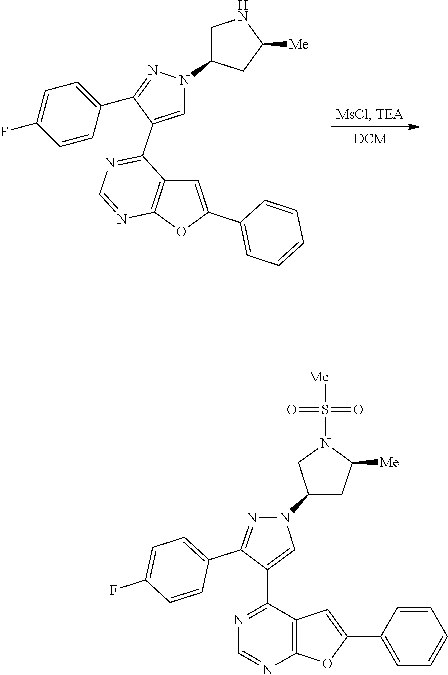 [SMILES: C[C@H]1C[C@@H](n2cc(-c3ncnc4oc(-c5ccccc5)cc34)c(-c3ccc(F)cc3)n2)CN1 ; C[C@H]1C[C@@H](n2cc(-c3ncnc4oc(-c5ccccc5)cc34)c(-c3ccc(F)cc3)n2)CN1S(C)(=O)=O]

To a stirred solution of 3-(4-fluorophenyl)-1-[(3R,5S)-5-methylpyrrolidin-3-yl]-4-{6-phenylfuro[2,3-d]pyrimidin-4-yl}pyrazole (90 mg, 0.205 mmol, 1 equiv.) in DCM (2.7 mL) was added TEA (62.17 mg, 0.615 mmol, 3 equiv.) under nitrogen atmosphere. To this mixture was added MsCl (28.15 mg, 0.246 mmol, 1.2 equiv.) dropwise at 0° C. The resulting mixture was stirred for additional 1 h at 0° C., then quenched by the addition of sat. NH₄Cl (aq., 5 mL) at 0° C. The resulting mixture was diluted with water (25 mL) and extracted with EtOAc (3×30 mL). The combined organic layers were washed with brine (3×30 mL) and dried over anhydrous Na₂SO₄. After filtration, the filtrate was concentrated under reduced pressure. The crude product was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 53% B to 63% B in 8 min, 63% B; Wavelength: 254 nm; RT1 (min): 7) to afford the title compound (41.6 mg, 39%) as a white solid. m/z (ESI+ve ion)=518.10 [M+H]⁺. ¹H-NMR (400 MHz, Chloroform-d) δ 9.01 (s, 1H), 8.56 (s, 1H), 7.64-7.61 (m, 2H), 7.56-7.52 (m, 2H), 7.50-7.45 (m, 3H), 7.14 (t, J=8.8 Hz, 2H), 6.02 (s, 1H), 4.96 (t, J=7.6 Hz, 1H), 4.18-4.14 (m, 2H), 3.94-3.88 (m, 1H), 3.01 (s, 3H), 2.91-2.83 (m, 1H), 2.48-2.39 (m, 1H), 1.52 (d, J=6.0 Hz, 3H).

### Example 200. 3-{3-(4-fluorophenyl)-4-[6-(1-methyl-1H-imidazol-4-yl)furo[2,3-d]pyrimidin-4-yl]-1H-pyrazol-1-yl}-16-thietane-1,1-dione

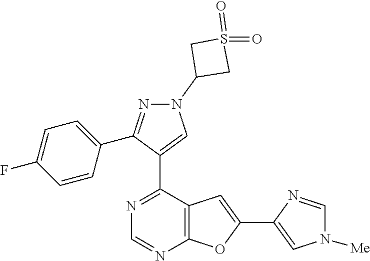 [SMILES: Cn1cnc(-c2cc3c(-c4cn(C5CS(=O)(=O)C5)nc4-c4ccc(F)cc4)ncnc3o2)c1]

### Step 1. 1-methyl-4-(tributylstannyl)imidazole

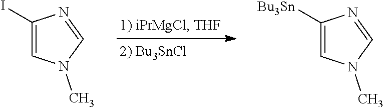

To a stirred solution of 4-iodo-1-methylimidazole (5 g, 24.038 mmol, 1 equiv.) in THF (50 mL, 617.138 mmol, 25.67 equiv.) at −10° C. under nitrogen was added ⁱPrMgCl·LiCl (18.03 mL, 36.057 mmol, 1.5 equiv, 2 M in THF) dropwise. The resulting mixture was stirred for 1 h at −10° C. under nitrogen. To the above mixture at −10° C. was added Bu₃SnCl (7.17 mL, 26.442 mmol, 1.1 equiv.) dropwise. The resulting mixture was stirred for an additional 16 h at room temperature, then quenched by the addition of sat. NH₄Cl (aq., 50 mL) and extracted with EtOAc (3×50 mL). The combined organic layers were washed with brine (3×50 mL) and dried over anhydrous Na₂SO₄. After filtration, the filtrate was concentrated under reduced pressure to give the title compound (8.9 g, quant) as a colorless oil. The crude product was used in the next step directly without further purification. m/z (ESI, +ve ion)=373.05 [M+H]⁺

### Step 2. 3-{3-(4-fluorophenyl)-4-[6-(1-methyl-1H-imidazol-4-yl)furo[2,3-d]pyrimidin-4-yl]-1H-pyrazol-1-yl}-16-thietane-1,1-dione

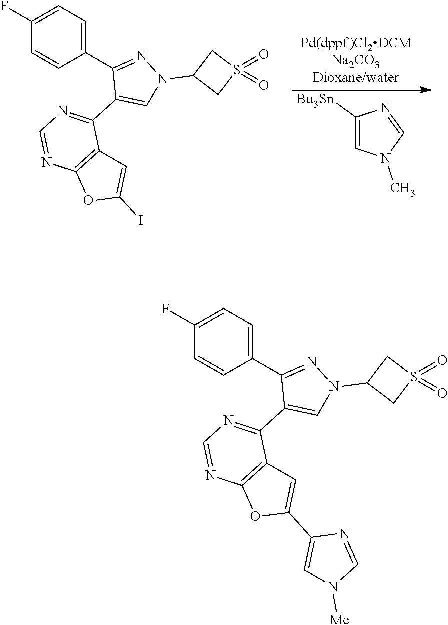

To a stirred solution of 3-[3-(4-fluorophenyl)-4-{6-iodofuro[2,3-d]pyrimidin-4-yl}pyrazol-1-yl]-1λ6-thietane-1,1-dione (Intermediate U, 5.5 g, 10.778 mmol, 1 equiv.) and Pd(dppf)Cl₂·DCM (0.88 g, 1.078 mmol, 0.1 equiv.) in 1,4-dioxane (82.5 mL) and H₂O (20.6 mL) at room temperature under nitrogen was added 1-methyl-4-(tributylstannyl)imidazole (6.00 g, 16.167 mmol, 1.5 equiv.) and Na₂CO₃ (3.39 g, 32.334 mmol, 3 equiv.). The resulting mixture was stirred for 3 h at 80° C. under nitrogen. The mixture was cooled to room temperature, then diluted with water (100 mL). The resulting mixture was extracted with EtOAc (3×100 mL). The combined organic layers were washed with brine (3×100 mL) and dried over anhydrous Na₂SO₄. After filtration, the filtrate was concentrated under reduced pressure. The residue was purified by silica gel column chromatography and eluted with DCM/MeOH (25/1) to afford crude product (3.6 g, contained 10% DCM). The crude product was re-crystallized from toluene (520 mL) to afford Example 200 (2.8 g, 58%) as a white solid. m/z (ESI, +ve ion)=465.00 [M+H]⁺, ¹H-NMR (400 MHz, Chloroform-d) δ 8.86 (s, 1H), 8.15 (s, 1H), 7.62-7.60 (m, 2H), 7.58 (s, 1H), 7.54 (s, 1H), 7.08-7.02 (m, 2H), 6.53 (s, 1H), 5.31-5.23 (m, 1H), 4.99-4.94 (m, 2H), 4.73-4.67 (m, 2H), 3.79 (s, 3H).

### Example 203. N-(3-{4-[1-(1,1-dioxo-1λ6-thietan-3-yl)-3-(4-fluorophenyl)-1H-pyrazol-4-yl]furo[2,3-d]pyrimidin-6-yl}phenyl)-N-methylmethanesulfonamide

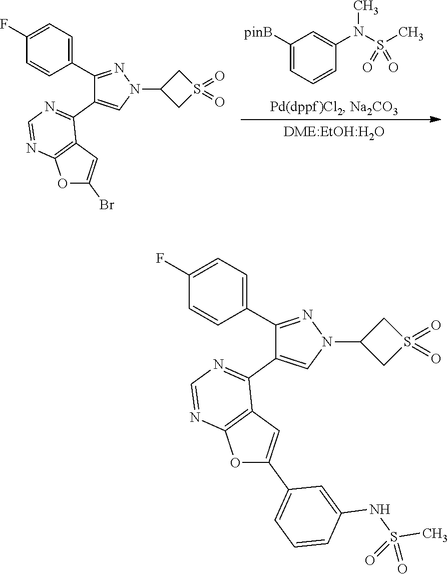

1,1′-Bis(diphenylphosphino)ferrocene dichloropalladium (II) (12.5 mg, 16.2 μmol, 0.1 equiv.) was added to a degassed mixture of 3-(4-{6-bromofuro[2,3-d]pyrimidin-4-yl}-3-(4-fluorophenyl)pyrazol-1-yl)-1λ6-thietane-1,1-dione (Intermediate S, 75.0 mg, 162 μmol, 1.0 equiv.), (3-(N-methylmethylsulfonamido)phenyl)boronic acid pinacol ester (66.3 mg, 202 μmol, 1.25 equiv.) and sodium carbonate (52.0 mg, 486 μmol, 3.0 equiv.) in a mixture of DME (2.25 mL), EtOH (750 μL) and H₂O (300 μL). The mixture was stirred for 75 minutes at 110° C. in a microwave reactor, then diluted with EtOAc (5 mL). The mixture was filtered and concentrated under reduced pressure. The crude material was purified by column chromatography (MeOH in DCM, 0-5%, a gradient elution), followed by reverse phase chromatography (C18 column, MeCN in water with 10 mM ammonium bicarbonate, 10-60%, a gradient elution) to afford the title compound (23.8 mg, 26%) as a solid. ¹H NMR (400 MHz, DMSO-d₆) δ 8.89 (s, 1H), 8.65 (s, 1H), 7.98 (dd, J=5.9, 3.6 Hz, 1H), 7.71-7.66 (m, 1H), 7.64-7.54 (m, 4H), 7.23-7.17 (m, 2H), 7.19 (s, 1H), 5.56-5.42 (m, 1H), 4.89 (app d, J=7.1 Hz, 4H), 3.11 (s, 3H), 3.11 (s, 3H). m/z (ESI, +ve ion)=568.2 [M+H]⁺.

### Example 205. 3-[3-(4-fluorophenyl)-4-(6-{3-[(4-methylpiperazin-1-yl)methyl]phenyl}furo[2,3-d]pyrimidin-4-yl)-1H-pyrazol-1-yl]-1λ6-thietane-1,1-dione

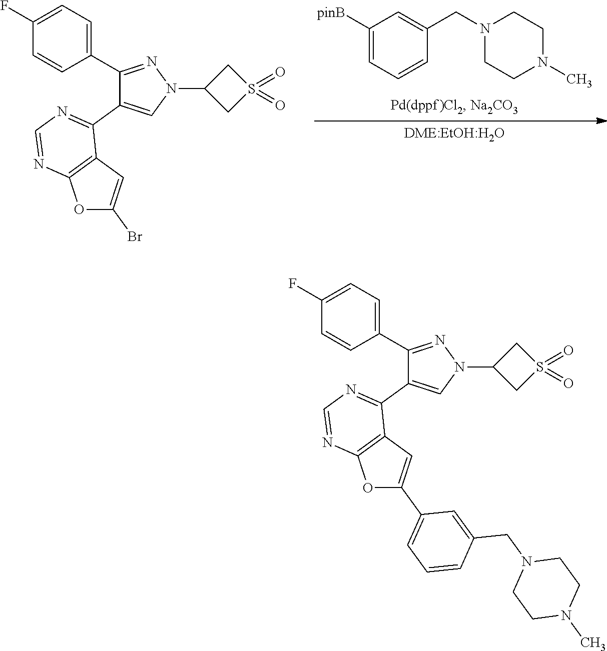

1,1′-Bis(diphenylphosphino)ferrocene dichloropalladium (II) (12.5 mg, 16.2 μmol, 0.1 equiv.) was added to a degassed mixture of 3-(4-{6-bromofuro[2,3-d]pyrimidin-4-yl}-3-(4-fluorophenyl)pyrazol-1-yl)-1,6-thietane-1,1-dione (Intermediate S, 75.0 mg, 162 μmol, 1.0 equiv.), 1-methyl-4-(3-(4,4,5,5-tetramethyl-1,3,2-dioxaborolan-2-yl)benzyl)piperazine (66.7 mg, 202 μmol, 1.25 equiv.) and sodium carbonate (52.0 mg, 486 μmol, 3.0 equiv.) in a mixture of DME (2.25 mL), EtOH (750 μL) and H₂O (300 μL). The mixture was stirred for 45 minutes at 110° C. in a microwave reactor then diluted with EtOAc (5 mL). The mixture was filtered and concentrated under reduced pressure. The crude material was purified by reverse phase chromatography (C18 column, MeCN in water with 10 mM ammonium formate, 10-40%, a gradient elution). The material was further purified by reverse phase chromatography (C18 column, MeCN in water with 10 mM ammonium bicarbonate, 10-40%, a gradient elution) to provide the title compound (37.5 mg, 40%) as a solid. ¹H NMR (400 MHz, DMSO-d₆) δ 8.81 (s, 1H), 8.79 (s, 1H), 7.79 (s, 1H), 7.78 (d, J=8.3 Hz, 1H), 7.64 (dd, J=8.5, 5.7 Hz, 2H), 7.51 (app t, J=7.5 Hz, 1H), 7.42 (d, J=7.4 Hz, 1H), 7.34 (s, 1H), 7.22 (app t, J=8.8 Hz, 2H), 5.58-5.45 (m, 1H), 5.00-4.83 (m, 4H), 3.54 (s, 2H), 2.46-2.25 (m, 8H), 2.17 (s, 3H). m/z (ESI, +ve ion)=573.3 [M+H]⁺.

### Example 208. 1-[5-{4-[3-(4-fluorophenyl)-1-(2-hydroxy-2-methylpropyl)-1H-pyrazol-4-yl]furo[2,3-d]pyrimidin-6-yl}-3,6-dihydropyridin-1 (2H)-yl]ethan-1-one

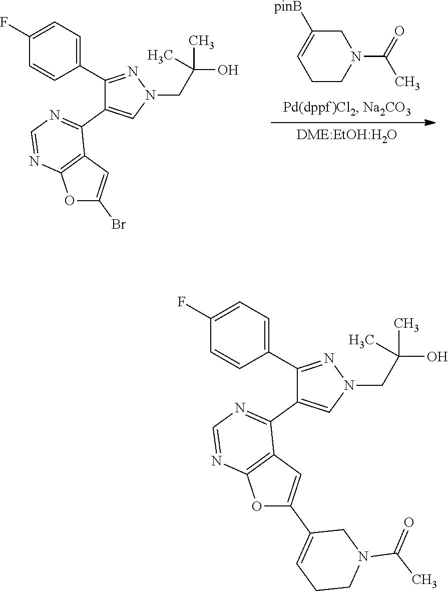

1-(4-(6-Bromofuro[2,3-d]pyrimidin-4-yl)-3-(4-fluorophenyl)-1H-pyrazol-1-yl)-2-methylpropan-2-ol (Product of Step 1 from Example 138, 113 mg, 262 μmol, 1.0 equiv.), 1-[5-(4,4,5,5-tetramethyl-1,3,2-dioxaborolan-2-yl)-3,6-dihydro-2H-pyridin-1-yl]ethanone (65.8 mg, 262 μmol, 1.0 equiv.), sodium carbonate (84.2 mg, 786 μmol, 3.0 equiv.) and 1,1′-bis(diphenylphosphino)ferrocene dichloropalladium (II) (20.2 mg, 26.2 μmol, 0.1 equiv.) were dissolved in DME (1.70 mL), EtOH (850 μL), and H₂O (425 μL). The mixture was purged with nitrogen then stirred at 85° C. for 16 hours. The reaction mixture was cooled to room temperature and diluted with water. The aqueous layer was extracted with EtOAc (3×20 mL). The combined organic layers were washed with brine and concentrated under reduced pressure. The crude material was purified by reverse phase chromatography (C18 column, MeCN in water with 10 mM ammonium formate, 10-100%, a gradient elution) to provide the title compound (56.7 mg, 46%) as a solid. ¹H NMR (400 MHz, DMSO-d₆, 90° C.) δ 8.71 (s, 1H), 8.38 (s, 1H), 7.60-7.52 (m, 2H), 7.17-7.07 (m, 2H), 6.68 (s, 1H), 6.54 (s, 1H), 4.55 (s, 1H), 4.25-4.13 (m, 4H), 3.57 (t, J=5.4 Hz, 2H), 2.37 (s, 2H), 2.06 (s, 3H), 1.18 (s, 6H). m/z (ESI, +ve ion)=476.4 [M+H]⁺.

### Example 210. 4-{1-[(3S,4S)-3-fluorooxan-4-yl]-3-(4-fluorophenyl)-1H-pyrazol-4-yl}-6-phenylfuro[2,3-d]pyrimidine

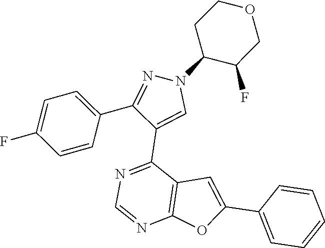 [SMILES: Fc1ccc(-c2nn([C@H]3CCOC[C@H]3F)cc2-c2ncnc3oc(-c4ccccc4)cc23)cc1]

### Example 211. 4-{1-[(3R,4R)-3-fluorooxan-4-yl]-3-(4-fluorophenyl)-1H-pyrazol-4-yl}-6-phenylfuro[2,3-d]pyrimidine

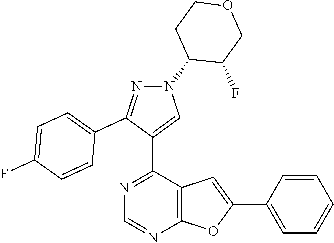 [SMILES: Fc1ccc(-c2nn([C@@H]3CCOC[C@@H]3F)cc2-c2ncnc3oc(-c4ccccc4)cc23)cc1]

The 4-(3-(4-fluorophenyl)-1-((cis)-3-fluorotetrahydro-2H-pyran-4-yl)-1H-pyrazol-4-yl)-6-phenylfuro[2,3-d]pyrimidine (Example 184, 72.7 mg, 0.159 mmol, 1 equiv.) was separated by Prep-Chiral-HPLC with the following conditions: Column: CHIRALPAK IG, 2×25 cm, 5 μm; Mobile Phase A: Hex:MTBE=1:1 (0.5% 2M NH₃-MeOH), Mobile Phase B: IPA-HPLC; Flow rate: 20 mL/min; Gradient: 10% B to 10% B in 17 min; Wavelength: 220/254 nm; RT1 (min): 11.87; RT2 (min): 14.75; Sample Solvent: MeOH:DCM=1:1-HPLC. The first product-containing fractions were collected and roto-evaporated in vacuo and lyophilized overnight to give Example 210 (27.8 mg, 38%) (absolute stereochemistry arbitrarily assigned) as a white solid. m/z (ESI, +ve ion)=459.30 [M+H]⁺. ¹H NMR (400 MHz, Chloroform-d) δ 8.94 (s, 1H), 8.26 (s, 1H), 7.68-7.66 (m, 2H), 7.59-7.54 (m, 2H), 7.48-7.40 (m, 3H), 7.14-7.08 (m, 2H), 6.17 (s, 1H), 5.07 (d, J=48.8 Hz, 1H), 4.78-4.66 (m, 1H), 4.38-4.26 (m, 2H), 3.78-3.66 (m, 2H), 2.69-2.64 (m, 1H), 2.19-2.03 (m, 1H). The second product-containing fractions were collected and roto-evaporated in vacuo and lyophilized to give Example 211 (72.5 mg, 37%) (absolute stereochemistry arbitrarily assigned) as a white solid. m/z (ESI, +ve ion)=459.25 [M+H]⁺. ¹H NMR (400 MHz, Chloroform-d) δ 8.95 (s, 1H), 8.29 (s, 1H), 7.68-7.65 (m, 2H), 7.59-7.54 (m, 2H), 7.48-7.40 (m, 3H), 7.14-7.08 (m, 2H), 6.16 (s, 1H), 5.08 (d, J=48.8 Hz, 1H), 4.78-4.66 (m, 1H), 4.38-4.26 (m, 2H), 3.78-3.65 (m, 2H), 2.69-2.64 (m, 1H), 2.19 (dd, J=12.8, 2.8 Hz, 1H).

### Example 212. 4-{3-(4-fluorophenyl)-1-[(3S,5R)-1-(methanesulfonyl)-5-methylpyrrolidin-3-yl]-1H-pyrazol-4-yl}-6-phenylfuro[2,3-d]pyrimidine

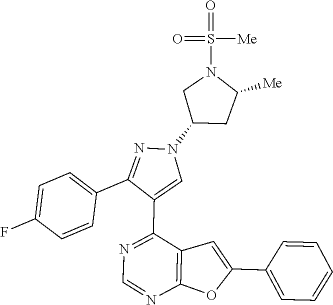 [SMILES: C[C@@H]1C[C@H](n2cc(-c3ncnc4oc(-c5ccccc5)cc34)c(-c3ccc(F)cc3)n2)CN1S(C)(=O)=O]

### Step 1. tert-butyl (2R,4R)-2-methyl-4-(tosyloxy)pyrrolidine-1-carboxylate

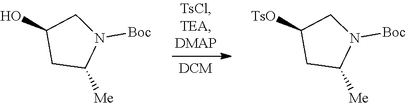 [SMILES: C[C@@H]1C[C@@H](O)CN1C(=O)OC(C)(C)C ; Cc1ccc(S(=O)(=O)O[C@@H]2C[C@@H](C)N(C(=O)OC(C)(C)C)C2)cc1]

To a stirred solution of tert-butyl (2R,4R)-4-hydroxy-2-methylpyrrolidine-1-carboxylate (300 mg, 1.491 mmol, 1 equiv.) in DCM (3 mL) at 20° C. under nitrogen was added TEA (0.41 mL, 2.982 mmol, 2 equiv.) and DMAP (18.21 mg, 0.149 mmol, 0.1 equiv.). To the mixture at 0° C. was added TsCl (340.99 mg, 1.789 mmol, 1.2 equiv.). The resulting mixture was stirred for an additional 16 hours at 20° C. then diluted with water (20 mL) and extracted with DCM (2×20 mL). The combined organic layers were washed with brine (2×20 mL) and dried over anhydrous Na₂SO₄. After filtration, the filtrate was concentrated under reduced pressure. The residue was purified by Prep-TLC (DCM/MeOH=10/1) to afford the title compound (401 mg, 76%) as a yellow oil. m/z (ESI, +ve ion)=373.15 [M+NH₄]+

### Step 2. tert-butyl (2R,4S)-4-(3-(4-fluorophenyl)-4-(6-phenylfuro[2,3-d]pyrimidin-4-yl)-1H-pyrazol-1-yl)-2-methylpyrrolidine-1-carboxylate

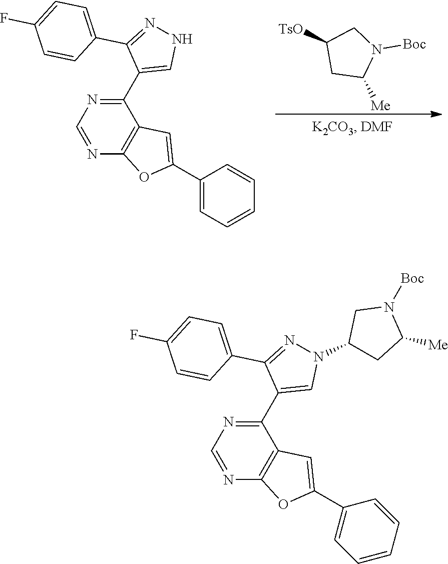 [SMILES: Fc1ccc(-c2n[nH]cc2-c2ncnc3oc(-c4ccccc4)cc23)cc1 ; Cc1ccc(S(=O)(=O)O[C@@H]2C[C@@H](C)N(C(=O)OC(C)(C)C)C2)cc1 ; C[C@@H]1C[C@H](n2cc(-c3ncnc4oc(-c5ccccc5)cc34)c(-c3ccc(F)cc3)n2)CN1C(=O)OC(C)(C)C]

To a stirred solution of 4-[3-(4-Fluorophenyl)-1H-pyrazol-4-yl]-6-phenylfuro[2,3-d]pyrimidine (Intermediate N, 385.15 mg, 1.081 mmol, 1.00 equiv.) and K₂CO₃ (298.74 mg, 2.162 mmol, 2 equiv.) in DMF (5 mL) at 20° C. under nitrogen was added tert-butyl (2R,4S)-2-methyl-4-[(4-methylbenzenesulfonyl)oxy]pyrrolidine-1-carboxylate (461 mg, 1.297 mmol, 1.2 equiv.). The resulting mixture was stirred for 16 hours at 60° C. under nitrogen then cooled down to 20° C. The reaction was quenched by the addition of water (5 mL) at 20° C. and then extracted with EtOAc (3×20 mL). The combined organic layers were washed with brine (3×20 mL) and dried over anhydrous Na₂SO₄. After filtration, the filtrate was concentrated under reduced pressure. The crude product was purified by Prep-HPLC with the following conditions (Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 82% B to 88% B in 8 min, 88% B; Wavelength: 254 nm; RT1 (min): 7.2 to afford the mixture (296 mg, contained 24% of N² regional isomer). The mixture was separated by Achiral SFC with the following conditions: Column: YMC-Actus SIL, 3×25 cm, 5 μm; Mobile Phase A: CO₂, Mobile Phase B: IPA:HEX=1:1 (0.1% 2M NH₃-MeOH); Flow rate: 60 mL/min; Gradient: isocratic 20% B; Column Temperature (° C.): 35; Back Pressure (bar): 100; Wavelength: 254 nm; RT1 (min): 4.78; RT2 (min): 6.23; Sample Solvent: EtOH-HPLC. The desired fractions (second peak) were combined and concentrated under reduced pressure to afford the title compound (207 mg, 35%) as yellow solid. m/z (ESI, +ve ion)=540.25 [M+H]⁺.

### Step 3. 3-(4-fluorophenyl)-1-[(3S,5R)-5-methylpyrrolidin-3-yl]-4-{6-phenylfuro[2,3-d]pyrimidin-4-yl}pyrazole

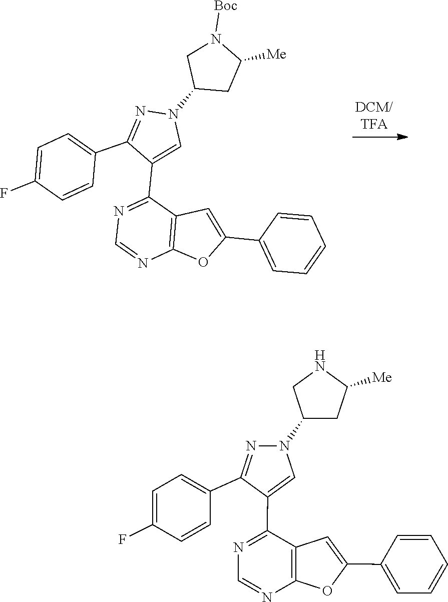 [SMILES: C[C@@H]1C[C@H](n2cc(-c3ncnc4oc(-c5ccccc5)cc34)c(-c3ccc(F)cc3)n2)CN1C(=O)OC(C)(C)C ; C[C@@H]1C[C@H](n2cc(-c3ncnc4oc(-c5ccccc5)cc34)c(-c3ccc(F)cc3)n2)CN1]

To a stirred solution of tert-butyl (2R,4S)-4-[3-(4-fluorophenyl)-4-{6-phenylfuro[2,3-d]pyrimidin-4-yl}pyrazol-1-yl]-2-methylpyrrolidine-1-carboxylate (207 mg, 0.384 mmol, 1 equiv.) in DCM (6 mL) at 20° C. under nitrogen was added TFA (2 mL). The resulting mixture was stirred for 30 min at 20° C. then concentrated under reduced pressure to give the title compound, TFA salt (160 mg, 95%) as a yellow oil. m/z (ESI, +ve ion)=440.15 [M+H]+

### Step 4. 4-{3-(4-fluorophenyl)-1-[(3S,5R)-1-(methanesulfonyl)-5-methylpyrrolidin-3-yl]-1H-pyrazol-4-yl}-6-phenylfuro[2,3-d]pyrimidine

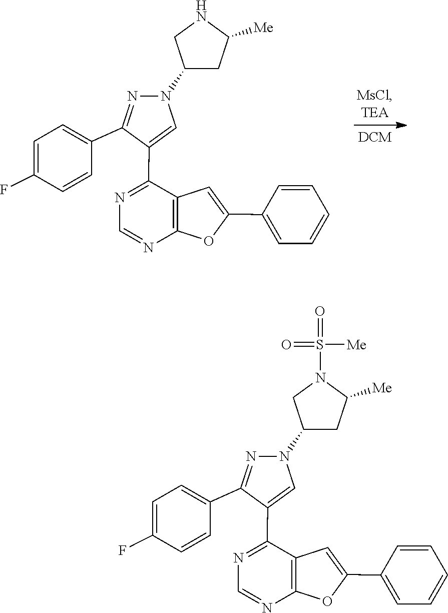 [SMILES: C[C@@H]1C[C@H](n2cc(-c3ncnc4oc(-c5ccccc5)cc34)c(-c3ccc(F)cc3)n2)CN1 ; C[C@@H]1C[C@H](n2cc(-c3ncnc4oc(-c5ccccc5)cc34)c(-c3ccc(F)cc3)n2)CN1S(C)(=O)=O]

To a stirred solution of 3-(4-fluorophenyl)-1-[(3S,5R)-5-methylpyrrolidin-3-yl]-4-{6-phenylfuro[2,3-d]pyrimidin-4-yl}pyrazole (420 mg, 0.956 mmol, 1 equiv.) and TEA (0.27 mL, 1.912 mmol, 2 equiv.) in DCM (5 mL) at 0° C. under nitrogen was added MsCl (0.11 mL, 1.434 mmol, 1.5 equiv.) dropwise. After the resulting mixture was stirred for 16 h at room temperature under nitrogen, it was quenched by the addition of water (5 mL) at 20° C. and extracted with DCM (3×5 mL). The combined organic layers were washed with brine (3×5 mL) and dried over anhydrous Na₂SO₄. After filtration, the filtrate was concentrated under reduced pressure. The crude product was purified by Prep-HPLC with the following conditions (Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 53% B to 68% B in 8 min, 68% B; Wavelength: 254 nm; RT1 (min): 6.83 to afford Example 212 (127 mg, 26%) as a white solid. m/z (ESI+ve ion)=518.10 [M+H]⁺. ¹H-NMR (400 MHz, Chloroform-d) δ 8.95 (s, 1H), 8.25 (s, 1H), 7.66-7.64 (m, 2H), 7.56-7.53 (m, 2H), 7.48-7.40 (m, 3H), 7.13-7.09 (t, J=16 Hz, 2H), 6.09 (s, 1H), 4.97-4.89 (m, 1H), 4.19-4.13 (m, 2H), 3.92-3.87 (m, 1H), 2.98 (s, 3H), 2.87-2.80 (m, 1H), 2.46-2.39 (m, 1H), 1.52-1.51 (d, J=4 Hz, 3H).

### Example 215. 6-{3-(4-fluorophenyl)-1-[3-(methanesulfonyl)propyl]-1H-pyrazol-4-yl}-8-phenyl-9H-purine

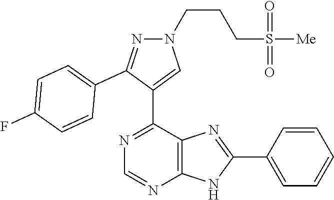 [SMILES: CS(=O)(=O)CCCn1cc(-c2ncnc3[nH]c(-c4ccccc4)nc23)c(-c2ccc(F)cc2)n1]

### Step 1. 6-[3-(4-fluorophenyl)-1-(3-methanesulfonylpropyl)pyrazol-4-yl]-9-[(4-methoxyphenyl)methyl]-8-phenylpurine

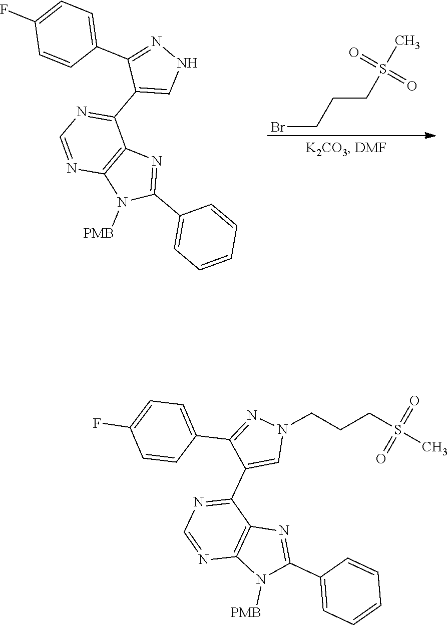

To a stirred mixture of 6-[3-(4-fluorophenyl)-1H-pyrazol-4-yl]-9-[(4-methoxyphenyl)methyl]-8-phenylpurine (Intermediate R, 100 mg, 0.210 mmol, 1 equiv.) and K₂CO₃ (58.01 mg, 0.420 mmol, 2 equiv.) in DMF (2.5 mL) at room temperature under nitrogen was added 1-bromo-3-methanesulfonylpropane (50.64 mg, 0.252 mmol, 1.2 equiv.) dropwise. The resulting mixture was stirred for 2 h at room temperature under nitrogen then quenched by the addition of water (10 mL) at room temperature. The resulting mixture was extracted with EtOAc (3×10 mL). The combined organic layers were washed with brine (2×15 mL) and dried over anhydrous Na₂SO₄. After filtration, the filtrate was concentrated under reduced pressure. The residue was purified by silica gel column chromatography, which eluted with PE/EtOAc (1/2) to afford the title compound (66 mg, 53%) as a yellow solid. m/z (ESI, +ve ion)=597.25 [M+H]+

### Step 2. 6-{3-(4-fluorophenyl)-1-[3-(methanesulfonyl)propyl]-1H-pyrazol-4-yl}-8-phenyl-9H-purine

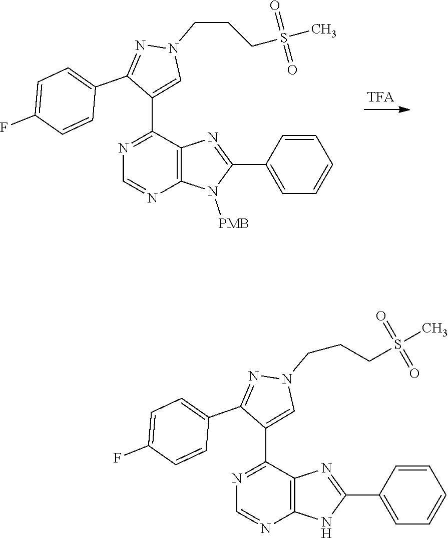

A mixture of 6-[3-(4-fluorophenyl)-1-(3-methanesulfonylpropyl)pyrazol-4-yl]-9-[(4-methoxyphenyl)methyl]-8-phenylpurine (60 mg, 0.101 mmol, 1 equiv.) in TFA (1.5 mL) was stirred for 5 h at room temperature under nitrogen. The resulting mixture was concentrated under vacuum. The residue was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 26% B to 36% B in 8 min, 36% B; Wavelength: 254 nm; RT1 (min): 7) to afford Example 215 (21.2 mg, 44%) as a white solid. m/z (ESI, +ve ion)=477.05 [M+H]⁺. ¹H NMR (400 MHz, DMSO-d₆) δ 13.88 (s, 1H), 9.03 (s, 1H), 8.66 (s, 1H), 8.33-8.25 (m, 2H), 7.78-7.68 (m, 2H), 7.65-7.54 (m, 3H), 7.25-7.14 (m, 2H), 4.47 (t, J=6.8 Hz, 2H), 3.29-3.21 (m, 2H), 3.03 (s, 3H), 2.36 (m, 2H).

### Example 216. 3-{3-(4-fluorophenyl)-4-[6-(1H-imidazol-4-yl)furo[2,3-d]pyrimidin-4-yl]-1H-pyrazol-1-yl}-16-thietane-1,1-dione

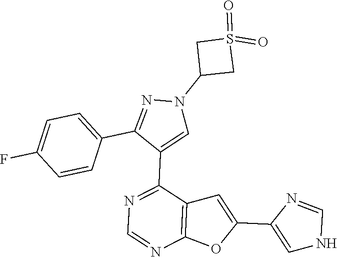 [SMILES: O=S1(=O)CC(n2cc(-c3ncnc4oc(-c5c[nH]cn5)cc34)c(-c3ccc(F)cc3)n2)C1]

### Step 1: 3-[3-(4-fluorophenyl)-4-{6-[1-(triphenylmethyl)imidazol-4-yl]furo[2,3-d]pyrimidin-4-yl}pyrazol-1-yl]-1λ6-thietane-1,1-dione

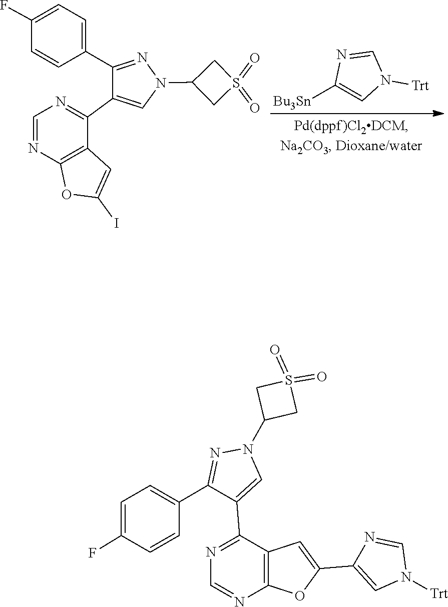

To a stirred solution of 3-[3-(4-fluorophenyl)-4-{6-iodofuro[2,3-d]pyrimidin-4-yl}pyrazol-1-yl]-1λ6-thietane-1,1-dione (Intermediate U, 200 mg, 0.392 mmol, 1 equiv.) and 4-(tributylstannyl)-1-(triphenylmethyl)imidazole (281.94 mg, 0.470 mmol, 1.2 equiv.) in 1,4-dioxane (4 mL) and water (1 mL) at room temperature under nitrogen was added Na₂CO₃ (83.08 mg, 0.784 mmol, 2 equiv.) and Pd(dppf)Cl₂·DCM (31.93 mg, 0.039 mmol, 0.1 equiv.). The resulting mixture was stirred for an additional 4 h at 80° C. then cooled to room temperature and quenched with water (20 mL). The resulting mixture was extracted with EtOAc (3×20 mL). The combined organic layers were washed with brine (2×10 mL) and dried over anhydrous Na₂SO₄. After filtration, the filtrate was concentrated under reduced pressure. The residue was purified by silica gel column chromatography and eluted with PE/EtOAc (1/1) to afford the title compound (200 mg, 74%) as a white solid. m/z (ESI, +ve ion)=693.30 [M+H]⁺.

### Step 2: 3-{3-(4-fluorophenyl)-4-[6-(1H-imidazol-4-yl)furo[2,3-d]pyrimidin-4-yl]-1H-pyrazol-1-yl}-16-thietane-1,1-dione

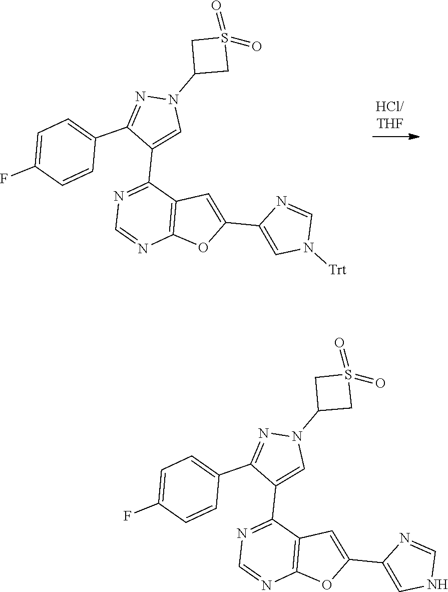

A solution of 3-[3-(4-fluorophenyl)-4-{6-[1-(triphenylmethyl)imidazol-4-yl]furo[2,3-d]pyrimidin-4-yl}pyrazol-1-yl]-1,6-thietane-1,1-dione (190 mg, 0.274 mmol, 1 equiv.) and HCl (6 M aq., 0.4 mL) in THF (2 mL) was stirred for 2 h at room temperature under nitrogen. The resulting mixture was made basic to pH 8 with saturated NaHCO₃ (aq.) and then extracted with EtOAc (3×10 mL). The combined organic layers were washed with brine (2×10 mL) and dried over anhydrous Na₂SO₄. After filtration, the filtrate was concentrated under reduced pressure. The crude product (220 mg) was purified by Prep-HPLC with the following conditions (Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 28% B to 40% B in 8 min, 40% B; Wavelength: 254 nm; RT1 (min): 6.72) to afford Example 216 (39.3 mg, 32%) as an off-white solid. m/z (ESI, +ve ion)=451.00 [M+H]⁺. ¹H NMR (400 MHz, DMSO-d₆) δ 12.60 (s, 1H), 8.78-8.73 (m, 2H), 7.87 (s, 1H), 7.80 (s, 1H), 7.65-7.62 (m, 2H), 7.23-7.19 (m, 2H), 6.95 (s, 1H), 5.54-5.51 (m, 1H), 4.95-4.83 (m, 4H).

### Example 217. 3-{3-(4-fluorophenyl)-4-[6-(1-methyl-1H-imidazol-5-yl)furo[2,3-d]pyrimidin-4-yl]-1H-pyrazol-1-yl}-16-thietane-1,1-dione

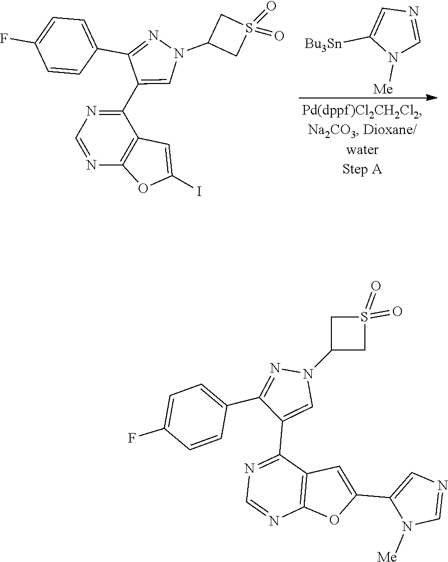

To a stirred solution of 3-[3-(4-fluorophenyl)-4-{6-iodofuro[2,3-d]pyrimidin-4-yl}pyrazol-1-yl]-1,6-thietane-1,1-dione (Intermediate U, 180 mg, 0.353 mmol, 1 equiv.) and 1-methyl-5-(tributylstannyl)imidazole (157.11 mg, 0.424 mmol, 1.2 equiv.) in 1,4-dioxane (3.2 mL) and H₂O (0.8 mL) at room temperature was added Na₂CO₃ (112.16 mg, 1.059 mmol, 3 equiv.) and Pd(dppf)Cl₂·CH₂Cl₂ (28.74 mg, 0.035 mmol, 0.1 equiv.). The resulting mixture was stirred for 3 h at 80° C. under nitrogen then cooled to room temperature. The reaction was quenched by the addition of water (10 mL) at room temperature and extracted with EtOAc (3×10 mL). The combined organic layers were washed with brine (3×10 mL) and dried over anhydrous Na₂SO₄. After filtration, the filtrate was concentrated under reduced pressure. The crude product (135 mg) was purified by Prep-HPLC with the following conditions (Column: YMC-Actus Triart C18 ExRS, 30×150 mm, 5 μm; Mobile Phase A: water (10 mmol/L NH₄HCO₃), Mobile Phase B: ACN; Flow rate: 60 mL/min; Gradient: 29% B to 29% B in 12 min, 29% B; Wavelength: 254 nm; RT1 (min): 10.2) to afford Example 217 (35.1 mg, 21%) as a white solid. m/z (ESI, +ve ion)=465.10 [M+H]⁺. H NMR (400 MHz, DMSO-d₆) δ 8.84 (s, 1H), 8.75 (s, 1H), 7.88 (s, 1H), 7.68-7.58 (m, 2H), 7.53 (s, 1H), 7.23 (t, J=8.0 Hz, 2H), 6.77 (s, 1H), 5.57-5.49 (m, 1H), 4.90 (d, J=8.0 Hz, 4H), 3.76 (s, 3H).

### Example 220. 4-{1-[(4,4-dimethyloxetan-2-yl)methyl]-3-(4-fluorophenyl)-1H-pyrazol-4-yl}-6-phenylfuro[2,3-d]pyrimidine

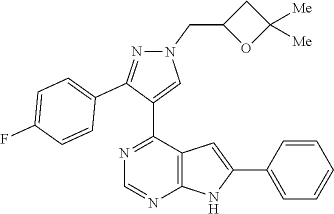 [SMILES: CC1(C)CC(Cn2cc(-c3ncnc4[nH]c(-c5ccccc5)cc34)c(-c3ccc(F)cc3)n2)O1]

### Step 1. (4,4-dimethyloxetan-2-yl)methyl 4-methylbenzenesulfonate

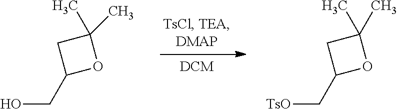

To a stirred solution of (4,4-dimethyloxetan-2-yl)methanol (200 mg, 1.722 mmol, 1 equiv.) and TsCl (393.88 mg, 2.066 mmol, 1.2 equiv.) in DCM (4 mL) at room temperature was added TEA (348.46 mg, 3.444 mmol, 2 equiv.) and DMAP (21.03 mg, 0.172 mmol, 0.1 equiv.). The resulting mixture was stirred for 16 h at room temperature under nitrogen then quenched by the addition of water (10 mL) at room temperature. The resulting mixture was extracted with EtOAc (3×10 mL). The combined organic layers were washed with brine (3×10 mL) and dried over anhydrous Na₂SO₄. After filtration, the filtrate was concentrated under reduced pressure. The residue was purified by silica gel column chromatography, eluted with PE/EtOAc (1/1) to afford the title compound (320 mg, 69%) as a white oil. No MS on LCMS. ¹H NMR (400 MHz, DMSO-d₆) δ 7.84-7.77 (m, 2H), 7.49 (d, J=8.0 Hz, 2H), 4.62-4.59 (m, 1H), 4.08 (d, J=4.2 Hz, 2H), 2.43 (s, 3H), 2.30-2.19 (m, 1H), 2.17-2.15 (m, 1H), 1.34 (s, 3H), 1.25 (s, 3H).

### Step 2. 4-{1-[(4,4-dimethyloxetan-2-yl)methyl]-3-(4-fluorophenyl)-1H-pyrazol-4-yl}-6-phenylfuro[2,3-d]pyrimidine

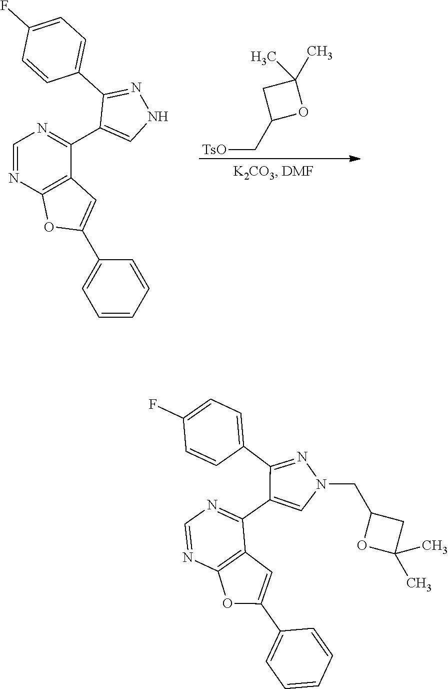 [SMILES: Fc1ccc(-c2n[nH]cc2-c2ncnc3oc(-c4ccccc4)cc23)cc1 ; Cc1ccc(S(=O)(=O)OCC2CC(C)(C)O2)cc1 ; CC1(C)CC(Cn2cc(-c3ncnc4oc(-c5ccccc5)cc34)c(-c3ccc(F)cc3)n2)O1]

To a stirred solution of 4-[3-(4-Fluorophenyl)-1H-pyrazol-4-yl]-6-phenylfuro[2,3-d]pyrimidine (Intermediate N, 200 mg, 0.561 mmol, 1 equiv.) and (4,4-dimethyloxetan-2-yl)methyl 4-methylbenzenesulfonate in DMF (2 mL) at room temperature was added K₂CO₃ (155.13 mg, 1.122 mmol, 2 equiv.). The resulting mixture was stirred for 18 h at 60° C. under nitrogen then cooled to room temperature. The reaction was quenched by the addition of water (10 mL) at room temperature and extracted with EtOAc (3×10 mL). The combined organic layers were washed with brine (3×10 mL) and dried over anhydrous Na₂SO₄. After filtration, the filtrate was concentrated under reduced pressure. The crude product (312 mg) was purified by Prep-HPLC with the following conditions (Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 55% B to 70% B in 8 min, 70% B; Wavelength: 254 nm; RT1 (min): 6.45 to afford Example 220 (135 mg, 52%) as a white solid. m/z (ESI, +ve ion)=455.30 [M+H]⁺. ¹H NMR (400 MHz, Methanol-d₄) δ 8.79 (d, J=2.0 Hz, 1H), 8.41 (d, J=9.6 Hz, 1H), 7.81-7.80 (m, 2H), 7.60-7.57 (m, 2H), 7.52-7.45 (m, 3H), 7.16-7.15 (m, 2H), 6.77 (d, J=16.4 Hz, 1H), 5.18-5.03 (m, 1H), 4.53 (d, J=4.8 Hz, 2H), 2.54-2.50 (m, 2H), 1.51 (s, 3H), 1.31 (d, J=2.8 Hz, 3H).

### Example 221. 4-[1-{[(2R)-4,4-dimethyloxetan-2-yl]methyl}-3-(4-fluorophenyl)-1H-pyrazol-4-yl]-6-phenylfuro[2,3-d]pyrimidine

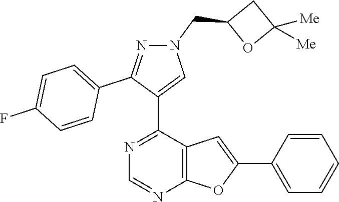 [SMILES: CC1(C)C[C@H](Cn2cc(-c3ncnc4oc(-c5ccccc5)cc34)c(-c3ccc(F)cc3)n2)O1]

### Example 222. 4-[1-{[(2S)-4,4-dimethyloxetan-2-yl]methyl}-3-(4-fluorophenyl)-1H-pyrazol-4-yl]-6-phenylfuro[2,3-d]pyrimidine

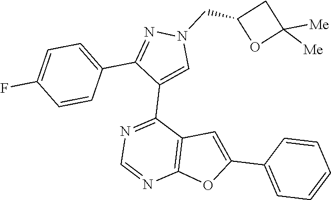 [SMILES: CC1(C)C[C@@H](Cn2cc(-c3ncnc4oc(-c5ccccc5)cc34)c(-c3ccc(F)cc3)n2)O1]

The racemic 1-[(4,4-dimethyloxetan-2-yl)methyl]-3-(4-fluorophenyl)-4-{6-phenylfuro[2,3-d]pyrimidin-4-yl}pyrazole (Example 220, 90 mg, 0.198 mmol, 1 equiv.) was separated by Prep-Chiral-HPLC with the following conditions: Column: CHIRAL ART Cellulose-SB, 2×25 cm, 5 μm; Mobile Phase A: Hex (0.5% 2M NH₃-MeOH)-HPLC, Mobile Phase B: EtOH-HPLC; Flow rate: 20 mL/min; Gradient: 20% B to 20% B in 12 min; Wavelength: 220/254 nm; RT1 (min): 9.47; RT2 (min): 10.74; Sample Solvent: MeOH:DCM=1:1-HPLC. The first product-containing fractions were concentrated under reduce pressure to give Example 221 (21.5 mg, 23%) (absolute stereochemistry arbitrarily assigned) as a white solid. m/z (ESI, +ve ion)=455.25 [M+H]⁺. ¹H NMR (400 MHz, Chloroform-d) δ 8.96 (s, 1H), 8.38 (s, 1H), 7.69-7.60 (m, 2H), 7.56-7.54 (m, 2H), 7.50-7.40 (m, 3H), 7.11 (t, J=8.4 Hz, 2H), 6.15 (s, 1H), 5.08 (s, 1H), 4.48 (d, J=4.8 Hz, 2H), 2.55-2.54 (m, 1H), 2.42-2.41 (m, 1H), 1.52 (s, 3H), 1.34 (s, 3H). The second product-containing fractions were concentrated under reduce pressure to give Example 222 (18.8 mg, 20%) (absolute stereochemistry arbitrarily assigned). m/z (ESI, +ve ion)=455.25 [M+H]⁺. ¹H NMR (400 MHz, Chloroform-d) δ 8.93 (s, 1H), 8.29 (s, 1H), 7.72-7.62 (m, 2H), 7.57 (t, J=6.4 Hz, 2H), 7.51-7.37 (m, 3H), 7.10 (t, J=8.4 Hz, 2H), 6.19 (s, 1H), 5.07 (s, 1H), 4.47 (d, J=4.8 Hz, 2H), 2.54-2.53 (m, 1H), 2.41-2.40 (m, 1H), 1.52 (s, 3H), 1.33 (s, 3H).

### Example 224. 3-{3-(4-fluorophenyl)-4-[6-(2-methyl-1,3-thiazol-5-yl)furo[2,3-d]pyrimidin-4-yl]-1H-pyrazol-1-yl}-16-thietane-1,1-dione

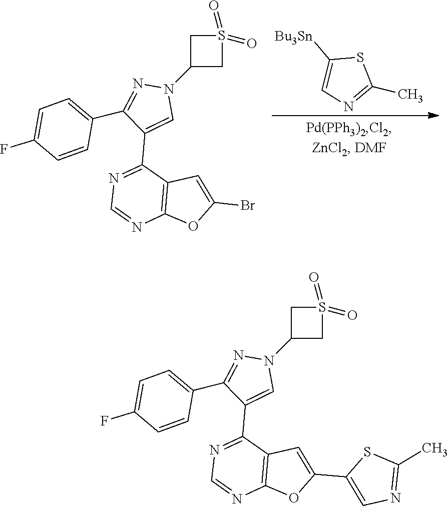

To a stirred solution of 3-(4-{6-bromofuro[2,3-d]pyrimidin-4-yl}-3-(4-fluorophenyl)pyrazol-1-yl 1,6-thietane-1,1-dione (Intermediate S, 100 mg, 0.216 mmol, 1 equiv.) and 2-methyl-5-(tributylstannyl)-1,3-thiazole (134.07 mg, 0.346 mmol, 1.6 equiv.) in DMF (2 mL) at room temperature under nitrogen was added Pd(PPh₃)₂Cl₂ (30.30 mg, 0.043 mmol, 0.2 equiv.) and ZnCl₂ (29.42 mg, 0.216 mmol, 1 equiv.). The resulting mixture was stirred for 4 h at 80° C. under nitrogen then cooled to room temperature. The reaction was quenched with water (40 mL) at room temperature and extracted with EtOAc (3×30 mL). The combined organic layers were washed with brine (2×10 mL) and dried over anhydrous Na₂SO₄. After filtration, the filtrate was concentrated under reduced pressure. The crude product (65 mg) was purified by Prep-HPLC with the following conditions (Column: XBridge Shield RP18 OBD Column, 30×150 mm, 5 μm; Mobile Phase A: water (10 mmol/L NH₄HCO₃), Mobile Phase B: ACN; Flow rate: 60 mL/min; Gradient: 40% B to 55% B in 8 min, 55% B; Wavelength: 254 nm; RT1 (min): 7.5 to afford Example 224 (41.5 mg, 40%) as a white solid. m/z (ESI, +ve ion)=482.00 [M+H]⁺. ¹H NMR (400 MHz, DMSO-d₆) δ 8.82 (s, 1H), 8.76 (s, 1H), 8.20 (s, 1H), 7.64-7.60 (m, 2H), 7.24-7.20 (m, 2H), 7.12 (s, 1H), 5.56-5.49 (m, 1H), 4.95-4.86 (m, 4H), 2.76 (s, 3H).

### Example 227. 4-[1-(5,5-dimethyloxolan-3-yl)-3-(4-fluorophenyl)-1H-pyrazol-4-yl]-6-phenylfuro[2,3-d]pyrimidine

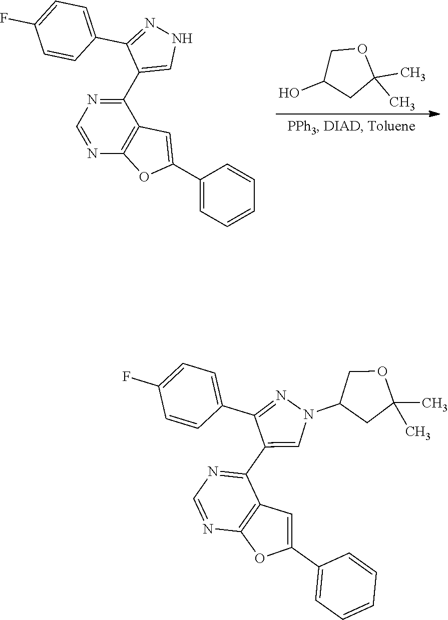

To a stirred solution of 4-[3-(4-fluorophenyl)-1H-pyrazol-4-yl]-6-phenylfuro[2,3-d]pyrimidine (Intermediate N, 200 mg, 0.561 mmol, 1.00 equiv.) and PPh₃ (220.81 mg, 0.842 mmol, 1.5 equiv.) in toluene (6 mL) at room temperature under nitrogen was added 5,5-dimethyloxolan-3-ol (78.23 mg, 0.673 mmol, 1.20 equiv.). To the above mixture was added DIAD (226.97 mg, 1.122 mmol, 2 equiv.) dropwise at 0° C. The resulting mixture was stirred for 2 h at 100° C. then cooled to room temperature. The resulting mixture was diluted with water (30 mL) and extracted with EtOAc (3×50 mL). The combined organic layers were washed with brine (3×50 mL) and dried over anhydrous Na₂SO₄. After filtration, the filtrate was concentrated under reduced pressure. The crude product was purified by Prep-HPLC with the following conditions (Column: YMC-Actus Triart C18 ExRS, 30×150 mm, 5 μm; Mobile Phase A: water (10 mmol/L NH₄HCO₃), Mobile Phase B: ACN; Flow rate: 60 mL/min; Gradient: 72% B to 72% B in 10 min, 72% B; Wavelength: 254 nm; RT1 (min): 8.8) to afford Example 227 (35 mg, 14%) as a white solid. m/z (ESI, +ve ion)=455.10 [M+H]⁺. ¹H NMR (400 MHz, Chloroform-d) δ 8.93 (s, 1H), 8.23 (s, 1H), 7.68-7.61 (m, 2H), 7.61-7.51 (m, 2H), 7.50-7.37 (m, 3H), 7.09 (t, J=8.8 Hz, 2H), 6.12 (s, 1H), 5.18-5.10 (m, 1H), 4.38-4.29 (m, 2H), 2.51-2.40 (m, 2H), 1.50 (s, 3H), 1.38 (s, 3H).

### Example 231. 4-{1-[2-(difluoromethoxy)ethyl]-3-(4-fluorophenyl)-1H-pyrazol-4-yl}-6-phenylfuro[2,3-d]pyrimidine

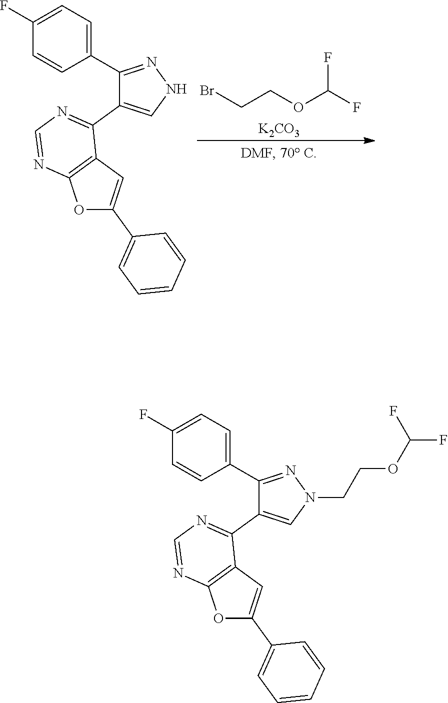

1-Bromo-2-(difluoromethoxy)ethane (73.9 mg, 401 μmol, 1.4 equiv.) was added to a mixture of 4-[3-(4-fluorophenyl)-1H-pyrazol-4-yl]-6-phenylfuro[2,3-d]pyrimidine (Intermediate N, 100 mg, 281 μmol, 1.0 equiv) and potassium carbonate (79.1 mg, 561 μmol, 2.0 equiv.) in DMF (2.80 mL). The mixture was stirred at 70° C. for 18 hours then diluted with water (20 mL) and extracted with EtOAc (3×10 mL). The combined organic layers were washed with water, brine, dried over Na₂SO₄, filtered, and concentrated under reduced pressure. The crude material was purified by prep-TLC (5% MeOH in DCM, isocratic elution), then by chiral-HPLC using the following conditions: 5 m, Cel-4, 10×250 mm; mode: isocratic; mobile phase: 20% MeOH+10 mM ammonium formate in water/supercritical CO₂; flow rate: 10 mL/min; backpressure: 150 bar; column temperature: 40° C.; run time (min): 13, to afford the title compound (57.0 mg, 45%) as a solid. ¹H NMR (400 MHz, DMSO-d₆) δ 8.78 (s, 1H), 8.67 (s, 1H), 7.94-7.86 (m, 2H), 7.56 (ddt, J=14.6, 9.4, 6.5 Hz, 5H), 7.35 (s, 2H), 7.20 (t, J=8.9 Hz, 2H), 6.74 (t, J=75.3 Hz, 1H), 4.55 (d, J=5.2 Hz, 2H), 4.38 (d, J=5.2 Hz, 2H). m/z (ESI, +ve ion)=451.3 [M+H]⁺.

### Example 232. 3-(3-{4-[1-(1,1-dioxo-1λ6-thietan-3-yl)-3-(4-fluorophenyl)-1H-pyrazol-4-yl]furo[2,3-d]pyrimidin-6-yl}phenyl)propanenitrile

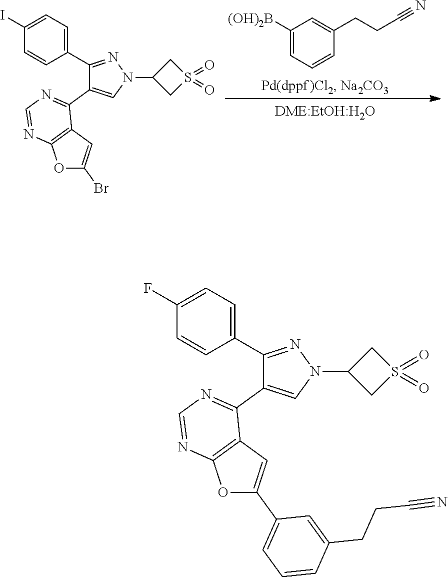

1,1′-Bis(diphenylphosphino)ferrocene dichloropalladium (II) (16.6 mg, 21.6 μmol, 0.1 equiv.) was added to a degassed mixture of 3-(4-{6-bromofuro[2,3-d]pyrimidin-4-yl}-3-(4-fluorophenyl)pyrazol-1-yl)-1λ6-thietane-1,1-dione (Intermediate S, 100 mg, 216 μmol, 1.0 equiv.), (3-(2-cyanoethyl)phenyl)boronic acid (48.3 mg, 276 μmol, 1.3 equiv.) and sodium carbonate (69.3 mg, 648 μmol, 3.0 equiv.) in a mixture of DME (3.0 mL), EtOH (1.0 mL) and H₂O (400 μL). The mixture was stirred for 45 minutes at 110° C. in a microwave reactor then diluted with EtOAc (5 mL). The mixture was filtered and concentrated under reduced pressure. The crude material was purified by column chromatography (EtOAc in hexanes, 50-100%, a gradient elution, followed by MeOH in DCM, 0-5%, a gradient elution). The material was further purified by reverse phase chromatography (C18 column, MeCN in water with 10 mM ammonium formate, 30-80%, a gradient elution) to provide the title compound (26.0 mg, 23%) as a solid. ¹H NMR (400 MHz, DMSO-d₆) δ 8.81 (s, 1H), 8.78 (s, 1H), 7.85 (s, 1H), 7.79 (d, J=7.8 Hz, 1H), 7.67-7.60 (m, 2H), 7.53 (t, J=7.7 Hz, 1H), 7.44 (d, J=7.7 Hz, 1H), 7.38 (s, 1H), 7.22 (t, J=8.9 Hz, 1H), 5.58-5.48 (m, 1H), 4.99-4.85 (m, 4H), 2.99 (t, J=6.9 Hz, 2H), 2.91 (t, J=6.8 Hz, 2H). m/z (ESI, +ve ion)=514.2 [M+H]⁺.

### Example 235. 3-{3-(4-fluorophenyl)-4-[6-(1,2-thiazol-3-yl)furo[2,3-d]pyrimidin-4-yl]-1H-pyrazol-1-yl}-1λ6-thietane-1,1-dione

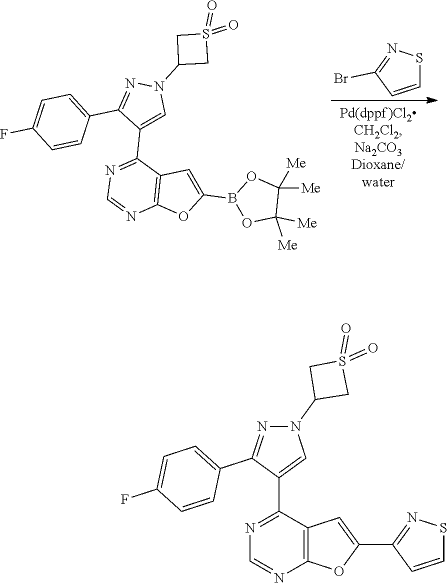 [SMILES: CC1(C)OB(c2cc3c(-c4cn(C5CS(=O)(=O)C5)nc4-c4ccc(F)cc4)ncnc3o2)OC1(C)C ; Brc1ccsn1 ; O=S1(=O)CC(n2cc(-c3ncnc4oc(-c5ccsn5)cc34)c(-c3ccc(F)cc3)n2)C1]

To a stirred solution of 3-[3-(4-fluorophenyl)-4-[6-(4,4,5,5-tetramethyl-1,3,2-dioxaborolan-2-yl)furo[2,3-d]pyrimidin-4-yl]pyrazol-1-yl]-1λ6-thietane-1,1-dione (Intermediate W, 300 mg, 0.588 mmol, 1 equiv.) and 3-bromo-1,2-thiazole (115.70 mg, 0.706 mmol, 1.2 equiv.) in 1,4-dioxane (3 mL) and H₂O (0.75 mL) at room temperature was added Na₂CO₃ (186.91 mg, 1.764 mmol, 3 equiv.) and Pd(dppf)Cl₂·CH₂Cl₂ (47.89 mg, 0.059 mmol, 0.1 equiv.). The resulting mixture was stirred for 3 h at 80° C. under nitrogen then cooled to room temperature. The reaction was quenched by the addition of water (10 mL) and then extracted with EtOAc (3×10 mL). The combined organic layers were washed with brine (3×10 mL) and dried over anhydrous Na₂SO₄. After filtration, the filtrate was concentrated under reduced pressure. The crude product (186 mg) was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 41% B to 51% B in 8 min, 51% B; Wavelength: 254 nm; RT1 (min): 7) to afford Example 235 (43.9 mg, 16%) as a white solid. m/z (ESI, +ve ion)=468.20 [M+H]⁺. H NMR (400 MHz, DMSO-d₆) δ 9.04 (s, 1H), 8.80 (d, J=4.0 Hz, 1H), 8.46 (s, 1H), 7.70 (d, J=4.0 Hz, 1H), 7.60-7.52 (m, 2H), 7.11 (t, J=8.0 Hz, 2H), 6.56 (s, 1H), 5.32 (m, 1H), 5.01-4.93 (m, 2H), 4.76-4.70 (m, 2H).

### Example 236. 3-{3-(4-fluorophenyl)-4-[6-(2-methyl-1,3-thiazol-4-yl)furo[2,3-d]pyrimidin-4-yl]-1H-pyrazol-1-yl}-16-thietane-1,1-dione

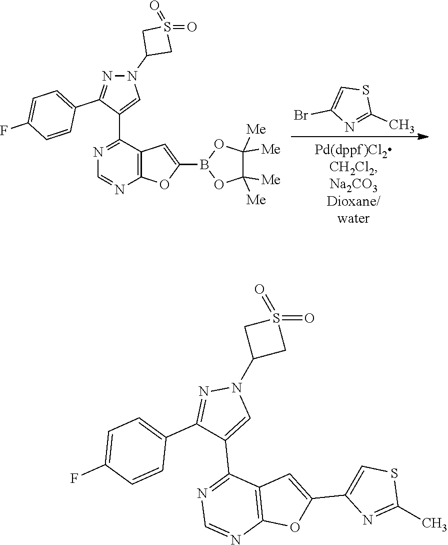

To a stirred solution of 3-[3-(4-fluorophenyl)-4-[6-(4,4,5,5-tetramethyl-1,3,2-dioxaborolan-2-yl)furo[2,3-d]pyrimidin-4-yl]pyrazol-1-yl]-1λ6-thietane-1,1-dione (Intermediate W, 200 mg, 0.392 mmol, 1 equiv.) and 4-bromo-2-methyl-1,3-thiazole (83.73 mg, 0.470 mmol, 1.2 equiv.) in 1,4-dioxane (2 mL) and H₂O (0.5 mL) was added Na₂CO₃ (124.61 mg, 1.176 mmol, 3 equiv.) and Pd(dppf)Cl₂·CH₂Cl₂ (31.92 mg, 0.039 mmol, 0.1 equiv.) at room temperature. The resulting mixture was stirred for 3 h at 80° C. under nitrogen then cooled to room temperature. The resulting mixture was quenched with water (10 mL) and extracted with EtOAc (3×10 mL). The combined organic layers were washed with brine (3×10 mL) and dried over anhydrous Na₂SO₄. After filtration, the filtrate was concentrated under reduced pressure. The crude product (120 mg) was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 41% B to 51% B in 8 min, 51% B; Wavelength: 254 nm; RT1 (min): 7) to afford Example 236 (37 mg, 20%) as an off-white solid. m/z (ESI, +ve ion)=482.20 [M+H]⁺. ¹H NMR (400 MHz, Chloroform-d) δ 8.95 (s, 1H), 8.30 (s, 1H), 7.70 (s, 1H), 7.63-7.49 (m, 2H), 7.09 (t, J=8.8 Hz, 2H), 6.59 (s, 1H), 5.30-5.27 (s, 1H), 4.97-4.95 (m, 2H), 4.72-4.71 (m, 2H), 2.78 (s, 3H).

### Example 237. 4-{1-[(3R)-5,5-dimethyloxolan-3-yl]-3-(4-fluorophenyl)-1H-pyrazol-4-yl}-6-phenylfuro[2,3-d]pyrimidine

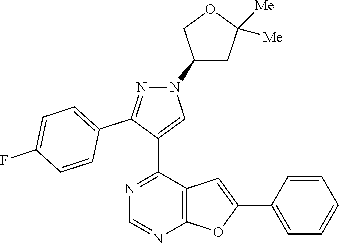 [SMILES: CC1(C)C[C@@H](n2cc(-c3ncnc4oc(-c5ccccc5)cc34)c(-c3ccc(F)cc3)n2)CO1]

### Example 238. 4-{1-[(3S)-5,5-dimethyloxolan-3-yl]-3-(4-fluorophenyl)-1H-pyrazol-4-yl}-6-phenylfuro[2,3-d]pyrimidine

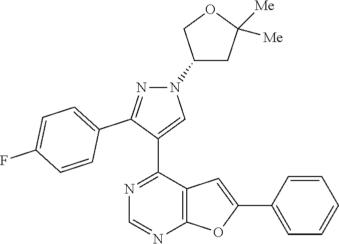 [SMILES: CC1(C)C[C@H](n2cc(-c3ncnc4oc(-c5ccccc5)cc34)c(-c3ccc(F)cc3)n2)CO1]

The racemic 1-(5,5-dimethyloxolan-3-yl)-3-(4-fluorophenyl)-4-{6-phenylfuro[2,3-d]pyrimidin-4-yl}pyrazole (Example 227, 35 mg, 0.077 mmol, 1 equiv.) was separated by Prep-Chiral-HPLC with the following conditions: Column: Lux 5 um Cellulose-4, 2.12×25 cm, 5 μm; Mobile Phase A: Hex (0.5% 2M NH₃-MeOH)-HPLC, Mobile Phase B: EtOH-HPLC; Flow rate: 20 mL/min; Gradient: 50% B to 50% B in 8 min; Wavelength: 220/254 nm; RT1 (min): 7.32; RT2 (min): 8.31; Sample Solvent: MeOH:MeCN=1:1-HPLC; Injection Volume: 0.6 mL. The first product-containing fractions were collected and roto-evaporated in vacuo and lyophilized overnight to give Example 237 (12.2 mg, 35%) (absolute stereochemistry arbitrarily assigned) as a white solid. m/z (ESI, +ve ion)=455.05 [M+H]⁺. ¹H NMR (400 MHz, Chloroform-d) δ 8.98 (s, 1H), 8.44 (s, 1H), 7.67-7.60 (m, 2H), 7.60-7.51 (m, 2H), 7.51-7.39 (m, 3H), 7.11 (t, J=8.4 Hz, 2H), 6.06 (s, 1H), 5.17-5.11 (m, 1H), 4.39-4.29 (m, 2H), 2.54-2.40 (m, 2H), 1.51 (s, 3H), 1.39 (s, 3H). The second product-containing fractions were collected and roto-evaporated in vacuo and lyophilized overnight to give Example 238 (13 mg, 37%) (absolute stereochemistry arbitrarily assigned) as a white solid. m/z (ESI, +ve ion)=455.05 [M+H]⁺. H NMR (400 MHz, Chloroform-d) δ 8.98 (s, 1H), 8.45 (s, 1H), 7.67-7.60 (m, 2H), 7.57-7.53 (m, 2H), 7.51-7.39 (m, 3H), 7.11 (t, J=8.8 Hz, 2H), 6.06 (s, 1H), 5.19-5.12 (m, 1H), 4.41-4.27 (m, 2H), 2.54-2.40 (m, 2H), 1.51 (s, 3H), 1.39 (s, 3H).

### Example 240. 3-{4-[6-(2,6-dimethylpyridin-3-yl)furo[2,3-d]pyrimidin-4-yl]-3-(4-fluorophenyl)-1H-pyrazol-1-yl}-16-thietane-1,1-dione

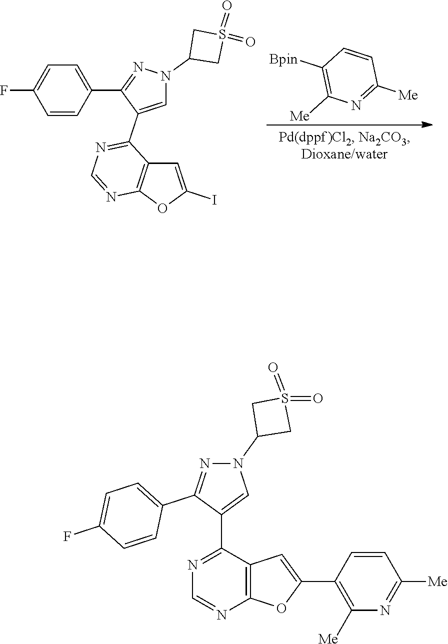

To a stirred solution of 2,6-dimethyl-3-(4,4,5,5-tetramethyl-1,3,2-dioxaborolan-2-yl)pyridine (68.53 mg, 0.294 mmol, 1.5 equiv.) and Na₂CO₃ (62.31 mg, 0.588 mmol, 3 equiv.) in dioxane (2 mL) and water (0.5 mL) at 80° C. under nitrogen was added 3-[3-(4-fluorophenyl)-4-{6-iodofuro[2,3-d]pyrimidin-4-yl}pyrazol-1-yl]-1,6-thietane-1,1-dione (Intermediate U, 100 mg, 0.196 mmol, 1.00 equiv.) and Pd(dppf)Cl₂ (14.34 mg, 0.020 mmol, 0.1 equiv.). The resulting mixture was stirred for 2 h at 80° C. then cooled to 20° C. The reaction was quenched with water (5 mL) at 20° C. and extracted with EtOAc (3×10 mL). The combined organic layers were washed with brine (3×10 mL) and dried over anhydrous Na₂SO₄. After filtration, the filtrate was concentrated under reduced pressure. The crude product (95 mg) was purified by Prep-HPLC with the following conditions (Column: XBridge Prep Phenyl OBD Column, 19×250 mm, 5 μm; Mobile Phase A: water (10 mmol/L NH₄HCO₃), Mobile Phase B: ACN; Flow rate: 25 mL/min; Gradient: 45% B to 55% B in 10 min, 55% B; Wavelength: 254 nm; RT1 (min): 9) to afford Example 240 (39.5 mg, 41%) as off-white solid. m/z (ESI, +ve ion)=490.00 [M+H]⁺. ¹H-NMR (400 MHz, DMSO-d₆) δ 8.88 (s, 1H), 8.76 (s, 1H), 8.06-8.04 (d, J=8 Hz, 1H), 7.64-7.61 (m, 2H), 7.30-7.28 (d, J=8 Hz, 1H), 7.24 (t, J=20 Hz, 2H), 6.89 (s, 1H), 5.55-5.50 (m, 1H), 4.92-4.85 (m, 4H), 3.31-3.28 (d, J=12 Hz, 3H), 2.57-2.50 (m, 3H).

### Example 242. 3-{4-[6-(1,3-dimethyl-1H-pyrazol-4-yl)furo[2,3-d]pyrimidin-4-yl]-3-(4-fluorophenyl)-1H-pyrazol-1-yl}-16-thietane-1,1-dione

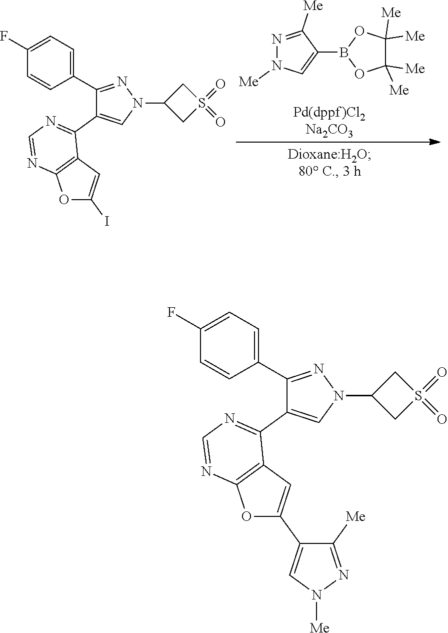

Sodium carbonate (29.1 mg, 0.27 mmol, 2.00 equiv), 3-[3-(4-fluorophenyl)-4-{6-iodofuro[2,3-d]pyrimidin-4-yl}pyrazol-1-yl]-1,6-thietane-1,1-dione (Intermediate U, 70 mg, 0.14 mmol, 1.00 equiv) and 1,3-dimethyl-4-(4,4,5,5-tetramethyl-1,3,2-dioxaborolan-2-yl)pyrazole (30.5 mg, 0.14 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (11.2 mg, 0.01 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by Column: Gemini 10 μm C18 110 Å LC Column, 250×30 mm; Mobile Phase A: water (10 mmol/L NH₄HCO₃), Mobile Phase B: ACN; Flow rate: 40 mL/min; Gradient: 35% B to 65% B in 24 min; Wavelength: 254 nm; RT1 (min): 13.4 to afford Example 242 (24 mg, 37%) as a white solid. m/z (ESI+ve ion)=479.15 [M+H]⁺.

¹H NMR (400 MHz, DMSO) δ 8.78 (s, 1H), 8.70 (s, 1H), 8.22 (s, 1H), 7.58 (d, J=7.2 Hz, 2H), 7.23 (s, 2H), 6.32 (s, 1H), 5.53 (d, J=7.7 Hz, 1H), 4.88 (s, 4H), 3.82 (s, 3H), 2.20 (s, 3H).

### Example 243. 3-{4-[6-(1,3-dimethyl-1H-pyrazol-5-yl)furo[2,3-d]pyrimidin-4-yl]-3-(4-fluorophenyl)-1H-pyrazol-1-yl}-16-thietane-1,1-dione

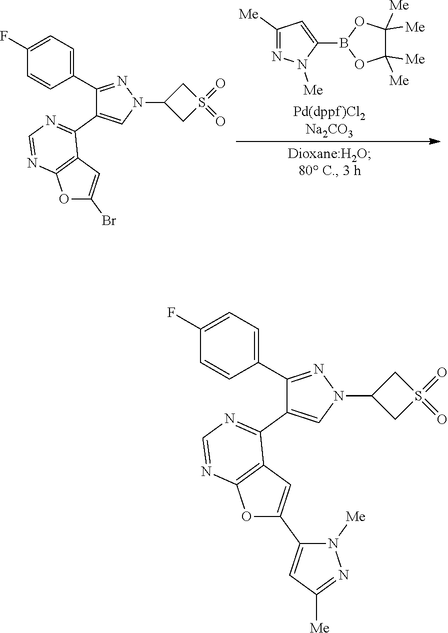 [SMILES: O=S1(=O)CC(n2cc(-c3ncnc4oc(Br)cc34)c(-c3ccc(F)cc3)n2)C1 ; Cc1cc(B2OC(C)(C)C(C)(C)O2)n(C)n1 ; Cc1cc(-c2cc3c(-c4cn(C5CS(=O)(=O)C5)nc4-c4ccc(F)cc4)ncnc3o2)n(C)n1]

Sodium carbonate (45.8 mg, 0.43 mmol, 2.00 equiv), 3-(4-{6-bromofuro[2,3-d]pyrimidin-4-yl}-3-(4-fluorophenyl)pyrazol-1-yl)-1,6-thietane-1,1-dione (Intermediate S, 100 mg, 0.22 mmol, 1.00 equiv) and 1,3-dimethyl-5-(4,4,5,5-tetramethyl-1,3,2-dioxaborolan-2-yl)pyrazole (47.9 mg, 0.22 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (17.6 mg, 0.02 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by Column: Gemini 10 μm C18 110 Å LC Column, 250×30 mm; Mobile Phase A: water (10 mmol/L NH₄HCO₃), Mobile Phase B: ACN; Flow rate: 40 mL/min; Gradient: 25% B to 65% B in 22 min; Wavelength: 254 nm; RT1 (min): 19.4 to afford Example 243 (70 mg, 68%) as a white solid. m/z (ESI+ve ion)=479.15 [M+H]⁺. ¹H NMR (400 MHz, DMSO) δ 8.86 (s, 1H), 8.76 (s, 1H), 7.61 (s, 2H), 7.22 (ddd, J=9.9, 6.2, 3.3 Hz, 2H), 6.95 (s, 1H), 6.61 (s, 1H), 5.52 (s, 1H), 4.90 (d, J=3.8 Hz, 4H), 3.90 (s, 3H), 2.20 (s, 3H).

### Example 244. 3-{3-(4-fluorophenyl)-4-[6-(1-methyl-1H-1,2,3-triazol-4-yl)furo[2,3-d]pyrimidin-4-yl]-1H-pyrazol-1-yl}-16-thietane-1,1-dione

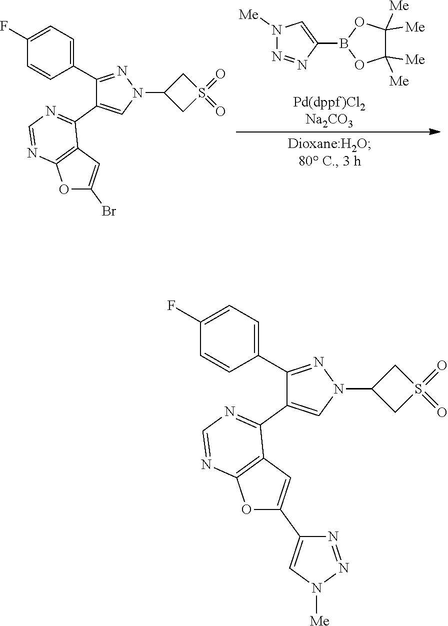 [SMILES: O=S1(=O)CC(n2cc(-c3ncnc4oc(Br)cc34)c(-c3ccc(F)cc3)n2)C1 ; Cn1cc(B2OC(C)(C)C(C)(C)O2)nn1 ; Cn1cc(-c2cc3c(-c4cn(C5CS(=O)(=O)C5)nc4-c4ccc(F)cc4)ncnc3o2)nn1]

Sodium carbonate (36.6 mg, 0.35 mmol, 2.00 equiv), 33-(4-{6-bromofuro[2,3-d]pyrimidin-4-yl}-3-(4-fluorophenyl)pyrazol-1-yl)-1,6-thietane-1,1-dione (Intermediate S, 80 mg, 0.17 mmol, 1.00 equiv) and 1-methyl-4-(4,4,5,5-tetramethyl-1,3,2-dioxaborolan-2-yl)triazole (36.1 mg, 0.17 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (14.1 mg, 0.02 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by Column: Gemini 10 μm C18 110 Å LC Column, 250×30 mm; Mobile Phase A: water (10 mmol/L NH₄HCO₃), Mobile Phase B: ACN; Flow rate: 40 mL/min; Gradient: 25% B to 65% B in 22 min; Wavelength: 254 nm; RT1 (min): 16.2 to afford Example 244 (32 mg, 40%) as a white solid. m/z (ESI+ve ion)=466.10 [M+H]⁺. ¹H NMR (400 MHz, DMSO) δ 8.81 (d, J=4.8 Hz, 2H), 8.72 (s, 1H), 7.63 (s, 2H), 7.32 (s, 1H), 7.22 (d, J=9.8 Hz, 2H), 5.53 (q, J=4.7 Hz, 1H), 5.02-4.79 (m, 4H), 4.23-4.09 (m, 3H).

### Example 245. 3-{3-(4-fluorophenyl)-4-[6-(2-methylphenyl)furo[2,3-d]pyrimidin-4-yl]-1H-pyrazol-1-yl}-16-thietane-1,1-dione

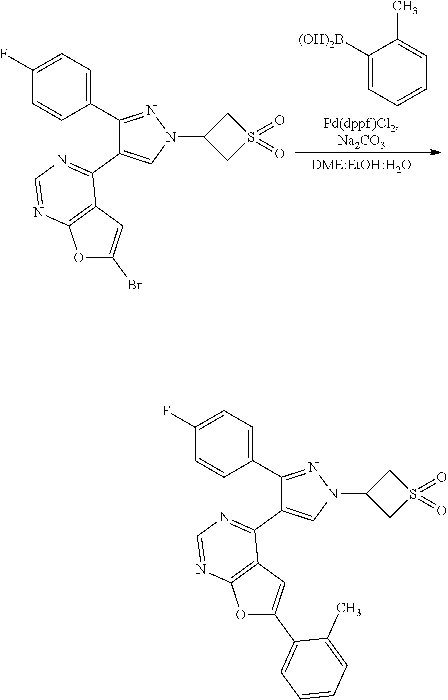

1,1′-Bis(diphenylphosphino)ferrocene dichloropalladium (II) (23.3 mg, 30.2 μmol, 0.1 equiv.) was added to a degassed mixture of 3-(4-{6-bromofuro[2,3-d]pyrimidin-4-yl}-3-(4-fluorophenyl)pyrazol-1-yl)-1λ6-thietane-1,1-dione (Intermediate S, 140 mg, 302 μmol, 1.0 equiv.), o-tolylboronic acid (55.4 mg, 387 μmol, 1.3 equiv.) and sodium carbonate (97.1 mg, 907 μmol, 3.0 equiv.) in a mixture of DME (4.20 mL), EtOH (1.40 mL) and H₂O (560 μL). The mixture was stirred for 45 minutes at 110° C. in a microwave reactor then diluted with water (20 mL) and extracted with EtOAc (3×10 mL). The combined organic layers were washed with brine, then dried over Na₂SO₄, filtered and concentrated. The crude material was purified by column chromatography (MeOH in DCM, 0-5%, a gradient elution), followed by reverse phase chromatography (C18 column, MeCN in water with 10 mM ammonium formate, 30-80%, a gradient elution) to provide the title compound (39.0 mg, 27%) as a solid. ¹H NMR (400 MHz, DMSO-d₆) δ 8.87 (s, 1H), 8.75 (s, 1H), 7.81-7.76 (m, 1H), 7.65-7.59 (m, 2H), 7.38 (dd, J=6.2, 2.5 Hz, 3H), 7.23 (t, J=8.9 Hz, 2H), 6.81 (s, 1H), 5.58-5.47 (m, 1H), 4.96-4.82 (m, 4H), 2.36 (s, 3H). m/z (ESI, +ve ion)=475.2 [M+H]⁺.

### Example 246. (1S,3S)-3-[3-(4-fluorophenyl)-4-(6-phenylfuro[2,3-d]pyrimidin-4-yl)-1H-pyrazol-1-yl]-1-imino-16-thiolan-1-one

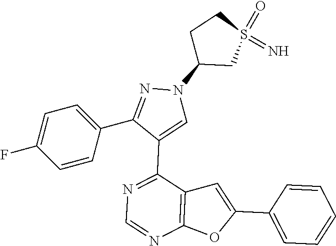 [SMILES: N=[S@]1(=O)CC[C@H](n2cc(-c3ncnc4oc(-c5ccccc5)cc34)c(-c3ccc(F)cc3)n2)C1]

### Example 247. (1R,3R)-3-[3-(4-fluorophenyl)-4-(6-phenylfuro[2,3-d]pyrimidin-4-yl)-1H-pyrazol-1-yl]-1-imino-1λ6-thiolan-1-one

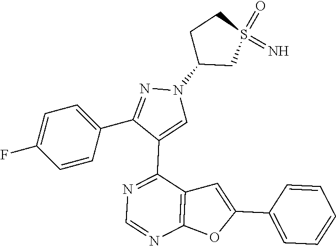 [SMILES: N=[S@]1(=O)CC[C@@H](n2cc(-c3ncnc4oc(-c5ccccc5)cc34)c(-c3ccc(F)cc3)n2)C1]

3-(3-(4-Fluorophenyl)-4-(6-phenylfuro[2,3-d]pyrimidin-4-yl)-1H-pyrazol-1-yl)-1-iminotetrahydro-1H-1λ6-thiophene 1-oxide (Example 161, 57.3 mg, 0.121 mmol, 1 equiv.) was separated by Prep-Chiral-HPLC with the following conditions: Column: CHIRALPAK IF, 2×25 cm, 5 μm; Mobile Phase A: Hex (0.5% 2M NH₃-MeOH)-HPLC, Mobile Phase B: EtOH:DCM=1:1-HPLC; Flow rate: 20 mL/min; Gradient: 30% B to 30% B in 27 min; Wavelength: 220/254 nm; RT1 (min): 18.60; RT2 (min): 21.83; Sample Solvent: MeOH:DCM=1:1-HPLC; The second product-containing fractions (major) were collected and separated by Prep-Chiral-HPLC again with the following conditions: Column: CHIRAL ART Cellulose-SC, 2×25 cm, 5 μm; Mobile Phase A: MtBE (0.5% 2M NH₃-MeOH)-HPLC, Mobile Phase B: EtOH:DCM=1:1-HPLC; Flow rate: 20 mL/min; Gradient: 30% B to 30% B in 13 min; Wavelength: 220/254 nm; RT1 (min): 9.26; RT2 (min): 12.40; Sample Solvent: MeOH:DCM=1:1-HPLC. The first product-containing fractions were collected and roto-evaporated in vacuo and lyophilized overnight to give Example 246 (23.2 mg, 40%) (absolute stereochemistry arbitrarily assigned) as a white solid. m/z (ESI, +ve ion)=474.25 [M+H]⁺. ¹H NMR (400 MHz, Chloroform-d) δ 8.94 (s, 1H), 8.32 (s, 1H), 7.66 (d, J=6.8 Hz, 2H), 7.55 (dd, J=8.4, 5.6 Hz, 2H), 7.48-7.41 (m, 3H), 7.11 (t, J=8.6 Hz, 2H), 6.15 (s, 1H), 5.38-5.30 (m, 1H), 3.90-3.79 (m, 2H), 3.73-3.66 (m, 1H), 3.42-3.35 (m, 1H), 2.99-2.89 (m, 2H), 2.41 (bs, 1H).

The second product-containing fractions were collected and roto-evaporated in vacuo and lyophilized overnight to give Example 247 (23.9 mg, 42%) (absolute stereochemistry arbitrarily assigned) as a white solid. m/z (ESI, +ve ion)=474.25 [M+H]⁺. ¹H NMR (400 MHz, Chloroform-d) δ 8.94 (s, 1H), 8.28 (s, 1H), 7.66 (dd, J=7.8, 1.8 Hz, 2H), 7.57-7.53 (m, 2H), 7.48-7.42 (m, 3H), 7.10 (t, J=8.6 Hz, 2H), 6.15 (s, 1H), 5.37-5.30 (m, 1H), 3.88-3.78 (m, 2H), 3.71-3.64 (m, 1H), 3.41-3.34 (m, 1H), 2.99-2.88 (m, 2H), 2.60 (bs, 1H).

### Example 248. 3-{3-(4-fluorophenyl)-4-[6-(1,3-thiazol-2-yl)furo[2,3-d]pyrimidin-4-yl]-1H-pyrazol-1-yl}-16-thietane-1,1-dione

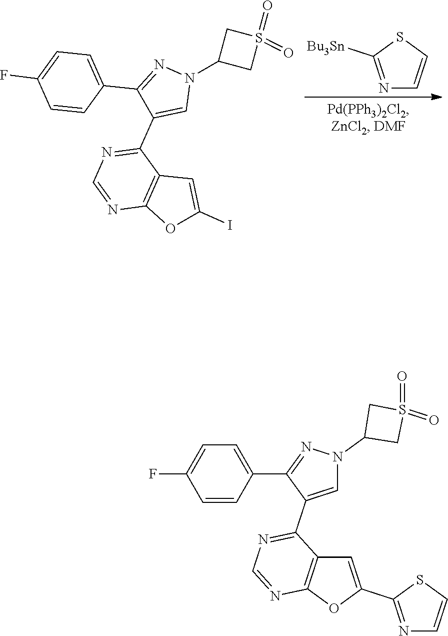

To a stirred solution of 3-[3-(4-fluorophenyl)-4-{6-iodofuro[2,3-d]pyrimidin-4-yl}pyrazol-1-yl]-1,6-thietane-1,1-dione (Intermediate U, 200 mg, 0.392 mmol, 1 equiv.) and Pd(PPh₃)₂Cl₂ (55.02 mg, 0.078 mmol, 0.2 equiv.) in DMF (3 mL) at room temperature under nitrogen was added 2-(tributylstannyl)-1,3-thiazole (175.98 mg, 0.470 mmol, 1.2 equiv.) and ZnCl₂ (53.41 mg, 0.392 mmol, 1 equiv.). The resulting mixture was stirred for 4 h at 80° C. under nitrogen then cooled to room temperature. The reaction was quenched with water (50 mL) and then extracted with EtOAc (3×40 mL). The combined organic layers were washed with brine (2×20 mL) and dried over anhydrous Na₂SO₄. After filtration, the filtrate was concentrated under reduced pressure. The crude product (200 mg) was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 38% B to 48% B in 8 min, 48% B; Wavelength: 254 nm; RT1 (min): 7) to afford Example 248 (27.2 mg, 14%) as an off-white solid. m/z (ESI, +ve ion)=467.95 [M+H]⁺. ¹H NMR (400 MHz, Chloroform-d) δ 9.01 (s, 1H), 8.32 (s, 1H), 7.98-7.97 (d, J=3.1 Hz, 1H), 7.58-7.53 (m, 3H), 7.13-7.08 (m, 2H), 6.67 (s, 1H), 5.35-5.27 (m, 1H), 4.99-4.94 (m, 2H), 4.76-4.70 (m, 2H).

### Example 250. 3-{3-(4-fluorophenyl)-4-[6-(1-methyl-1H-pyrazol-4-yl)furo[2,3-d]pyrimidin-4-yl]-1H-pyrazol-1-yl}-16-thietane-1,1-dione

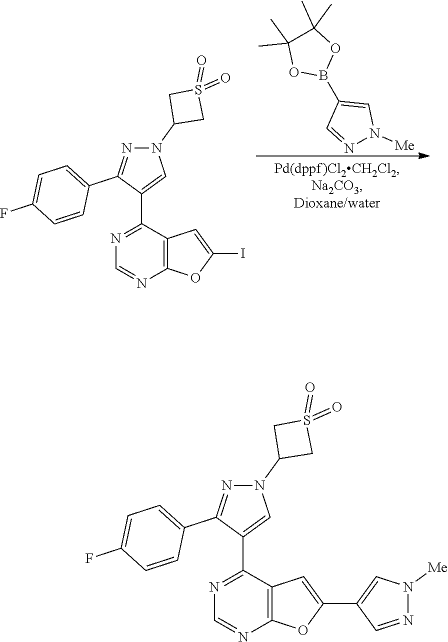 [SMILES: O=S1(=O)CC(n2cc(-c3ncnc4oc(I)cc34)c(-c3ccc(F)cc3)n2)C1 ; Cn1cc(B2OC(C)(C)C(C)(C)O2)cn1 ; Cn1cc(-c2cc3c(-c4cn(C5CS(=O)(=O)C5)nc4-c4ccc(F)cc4)ncnc3o2)cn1]

To a stirred solution of 3-[3-(4-fluorophenyl)-4-{6-iodofuro[2,3-d]pyrimidin-4-yl}pyrazol-1-yl]-1,6-thietane-1,1-dione (Intermediate U, 100 mg, 0.196 mmol, 1 equiv.) and 1-methyl-4-(4,4,5,5-tetramethyl-1,3,2-dioxaborolan-2-yl)pyrazole (48.93 mg, 0.235 mmol, 1.2 equiv.) in 1,4-dioxane (3.2 mL) and water (0.8 mL) were added Na₂CO₃ (62.31 mg, 0.588 mmol, 3 equiv.) and Pd(dppf)Cl₂·CH₂Cl₂ (15.96 mg, 0.020 mmol, 0.1 equiv.) at room temperature. The resulting mixture was stirred for 3 h at 80° C. under nitrogen then cooled to room temperature. The reaction was quenched by the addition of water (10 mL) and extracted with EtOAc (3×10 mL). The combined organic layers were washed with brine (3×10 mL) and dried over anhydrous Na₂SO₄. After filtration, the filtrate was concentrated under reduced pressure. The crude product (132 mg) was purified by Prep-HPLC with the following conditions (Column: Xselect CSH F-Phenyl OBD column, 19×250 mm, 5 μm; Mobile Phase A: water (0.1% FA), Mobile Phase B: ACN; Flow rate: 25 mL/min; Gradient: 48% B to 50% B in 8 min, 50% B; Wavelength: 254 nm; RT1 (min): 7) to afford Example 250 (38.1 mg, 40%) as an off-white solid. m/z (ESI, +ve ion)=465.20 [M+H]⁺. ¹H NMR (400 MHz, Chloroform-d) δ 8.90 (s, 1H), 8.26 (s, 1H), 7.69 (d, J=12 Hz, 2H), 7.56 (d, J=8.0 Hz, 2H), 7.09 (t, J=8.0 Hz, 2H), 5.81 (s, 1H), 5.31 (s, 1H), 4.95 (d, J=12.0 Hz, 2H), 4.72 (s, 2H), 3.98 (s, 3H).

### Example 251. 1-{4-[6-(1,3-dimethyl-1H-pyrazol-4-yl)furo[2,3-d]pyrimidin-4-yl]-3-(4-fluorophenyl)-1H-pyrazol-1-yl}-2-methylpropan-2-ol

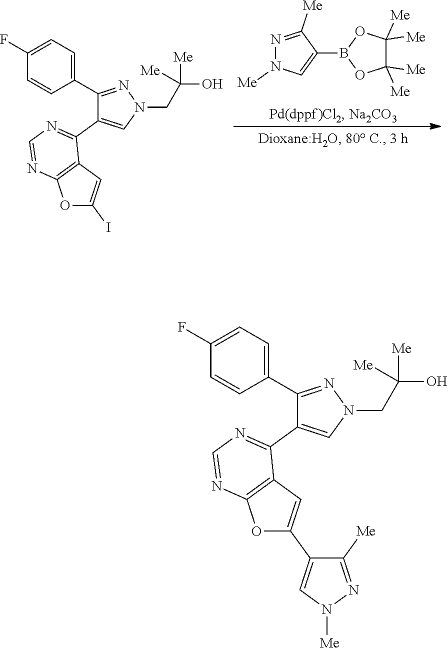 [SMILES: CC(C)(O)Cn1cc(-c2ncnc3oc(I)cc23)c(-c2ccc(F)cc2)n1 ; Cc1nn(C)cc1B1OC(C)(C)C(C)(C)O1 ; Cc1nn(C)cc1-c1cc2c(-c3cn(CC(C)(C)O)nc3-c3ccc(F)cc3)ncnc2o1]

Sodium carbonate (35.5 mg, 0.33 mmol, 2.00 equiv), 1-[3-(4-fluorophenyl)-4-{6-iodofuro[2,3-d]pyrimidin-4-yl}pyrazol-1-yl]-2-methylpropan-2-ol (Intermediate X, 80 mg, 0.17 mmol, 1.00 equiv) and 1,3-dimethyl-4-(4,4,5,5-tetramethyl-1,3,2-dioxaborolan-2-yl)pyrazole (37.1 mg, 0.17 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (13.7 mg, 0.02 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by Column: Gemini 10 μm C18 110 Å LC Column, 250×30 mm; Mobile Phase A: water (10 mmol/L NH₄HCO₃), Mobile Phase B: ACN; Flow rate: 40 mL/min; Gradient: 25% B to 70% B in 22 min; Wavelength: 254 nm; RT1 (min): 14.4 to afford Example 251 (34 mg, 46%) as a white solid. m/z (ESI+ve ion)=447.20 [M+H]⁺. ¹H NMR (400 MHz, DMSO) δ 8.75 (s, 1H), 8.38 (s, 1H), 8.20 (s, 1H), 7.54 (d, J=6.4 Hz, 2H), 7.20 (s, 2H), 6.24 (s, 1H), 4.86 (s, 1H), 4.18 (s, 2H), 3.82 (s, 3H), 2.17 (s, 3H), 1.18 (s, 6H).

### Example 252. 1-{3-(4-fluorophenyl)-4-[6-(1,3,5-trimethyl-1H-pyrazol-4-yl)furo[2,3-d]pyrimidin-4-yl]-1H-pyrazol-1-yl}-2-methylpropan-2-ol

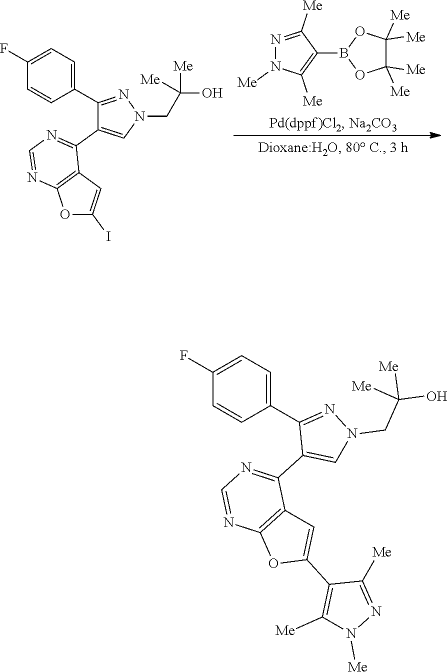 [SMILES: CC(C)(O)Cn1cc(-c2ncnc3oc(I)cc23)c(-c2ccc(F)cc2)n1 ; Cc1nn(C)c(C)c1B1OC(C)(C)C(C)(C)O1 ; Cc1nn(C)c(C)c1-c1cc2c(-c3cn(CC(C)(C)O)nc3-c3ccc(F)cc3)ncnc2o1]

Sodium carbonate (35.5 mg, 0.33 mmol, 2.00 equiv), 1-[3-(4-fluorophenyl)-4-{6-iodofuro[2,3-d]pyrimidin-4-yl}pyrazol-1-yl]-2-methylpropan-2-ol (Intermediate X, 80 mg, 0.17 mmol, 1.00 equiv) and 1,3,5-trimethyl-4-(4,4,5,5-tetramethyl-1,3,2-dioxaborolan-2-yl)pyrazole (39.5 mg, 0.17 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (13.7 mg, 0.02 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by Column: Gemini 10 μm C18 110 Å LC Column, 250×30 mm; Mobile Phase A: water (10 mmol/L NH₄HCO₃), Mobile Phase B: ACN; Flow rate: 40 mL/min; Gradient: 25% B to 65% B in 22 min; Wavelength: 254 nm; RT1 (min): 12.0 to afford Example 252 (36 mg, 47%) as a white solid. m/z (ESI+ve ion)=461.15 [M+H]⁺. ¹H NMR (400 MHz, DMSO) δ 8.77 (s, 1H), 8.37 (s, 1H), 7.60-7.45 (m, 2H), 7.28-7.02 (m, 2H), 6.15 (s, 1H), 4.85 (s, 1H), 4.17 (s, 2H), 3.71 (s, 3H), 2.37 (s, 3H), 2.16 (s, 3H), 1.17 (s, 6H).

### Example 253. 3-{3-(4-fluorophenyl)-4-[6-(6-methylpyridin-2-yl)furo[2,3-d]pyrimidin-4-yl]-1H-pyrazol-1-yl}-16-thietane-1,1-dione

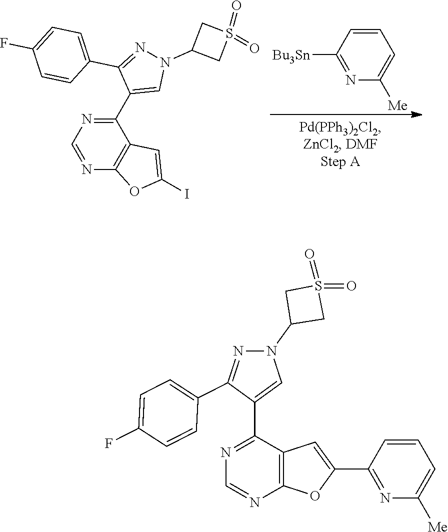

To a stirred solution of 3-[3-(4-fluorophenyl)-4-{6-iodofuro[2,3-d]pyrimidin-4-yl}pyrazol-1-yl]-1λ6-thietane-1,1-dione (Intermediate U, 100 mg, 0.196 mmol, 1 equiv.) and 2-methyl-6-(tributylstannyl)pyridine (119.83 mg, 0.314 mmol, 1.6 equiv.) in DMF (2 mL) at room temperature under nitrogen was added ZnCl₂ (26.6 mg, 0.196 mmol, 1 equiv.) and Pd(PPh₃)₂Cl₂ (6.8 mg, 0.039 mmol, 0.2 equiv.). The resulting mixture was stirred for 4 h at 80° C. under nitrogen then quenched with water (30 mL). The resulting mixture was extracted with EtOAc (3×20 mL). The combined organic layers were washed with brine (2×10 mL) and dried over anhydrous Na₂SO₄. After filtration, the filtrate was concentrated under reduced pressure. The crude product (200 mg) was purified by Prep-HPLC with the following conditions (Column: XSelect CSH Prep C18 OBD Column, 19×250 mm, 5 μm; Mobile Phase A: water (0.05% FA), Mobile Phase B: MeOH-HPLC; Flow rate: 25 mL/min; Gradient: 73% B to 74% B in 8 min, 74% B; Wavelength: 254 nm; RT1 (min): 6.6) to afford Example 253 (28 mg, 29%) as a white solid. m/z (ESI, +ve ion)=476.05 [M+H]⁺. ¹H NMR (400 MHz, Chloroform-d) δ 8.94 (s, 1H), 8.31 (s, 1H), 7.79 (s, 2H), 7.64-7.60 (m, 2H), 7.28-7.24 (m, 2H), 7.11-7.07 (m, 2H), 5.36-5.29 (m, 1H), 5.02-4.97 (m, 2H), 4.76-4.70 (m, 2H), 2.68 (s, 3H).

### Example 254. 1-{3-(4-fluorophenyl)-4-[6-(1-methyl-1H-imidazol-4-yl)furo[2,3-d]pyrimidin-4-yl]-1H-pyrazol-1-yl}-2-methylpropan-2-ol

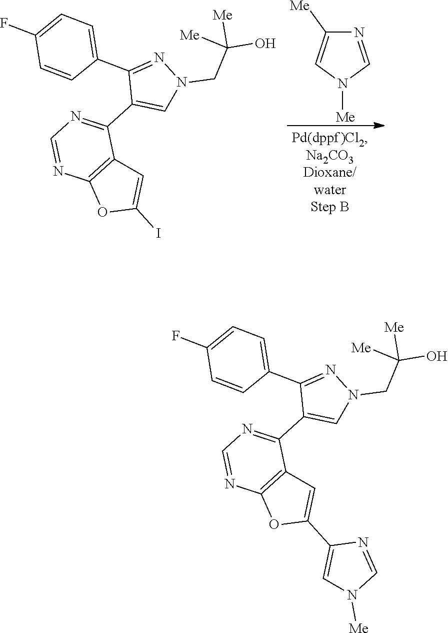 [SMILES: CC(C)(O)Cn1cc(-c2ncnc3oc(I)cc23)c(-c2ccc(F)cc2)n1 ; Cc1cn(C)cn1 ; Cn1cnc(-c2cc3c(-c4cn(CC(C)(C)O)nc4-c4ccc(F)cc4)ncnc3o2)c1]

To a stirred solution of 1-[3-(4-fluorophenyl)-4-{6-iodofuro[2,3-d]pyrimidin-4-yl}pyrazol-1-yl]-2-methylpropan-2-ol (Intermediate X, 120 mg, 0.251 mmol, 1 equiv.) and 1-methyl-4-(tributylstannyl)imidazole (111.75 mg, 0.301 mmol, 1.2 equiv.) in 1,4-dioxane (3.2 mL) and H₂O (0.8 mL) was added Na₂CO₃ (79.78 mg, 0.753 mmol, 3 equiv.) and Pd(dppf)Cl₂·CH₂Cl₂ (20.44 mg, 0.025 mmol, 0.1 equiv.) at room temperature. After the resulting mixture was stirred for 3 h at 80° C. under nitrogen, it was cooled down to room temperature. The reaction was quenched by the addition of water (10 mL) and extracted with EtOAc (3×10 mL). The combined organic layers were washed with brine (3×10 mL) and dried over anhydrous Na₂SO₄. After filtration, the filtrate was concentrated under reduced pressure. The crude product (230 mg) was purified by Prep-HPLC with the following conditions (Column: XSelect CSH Prep C18 OBD Column, 19×250 mm, 5 μm; Mobile Phase A: water (10 mmol/L NH₄HCO₃), Mobile Phase B: ACN; Flow rate: 25 mL/min; Gradient: 40% B to 40% B in 8 min, 40% B; Wavelength: 254 nm; RT1 (min): 8.4 to afford Example 254 (40.4 mg, 37%) as a white solid. m/z (ESI, +ve ion)=433.30 [M+H]⁺. ¹H-NMR (400 MHz, DMSO-d₆) δ 8.72 (s, 1H), 8.48 (s, 1H), 7.82-7.76 (m, 2H), 7.65-7.56 (m, 2H), 7.24-7.14 (m, 2H), 6.81 (s, 1H), 4.84 (s, 1H), 4.20 (s, 2H), 3.74 (s, 3H), 1.19 (s, 6H).

### Example 255. 1-{3-(4-fluorophenyl)-4-[6-(pyridin-3-yl)furo[2,3-d]pyrimidin-4-yl]-1H-pyrazol-1-yl}-2-methylpropan-2-ol

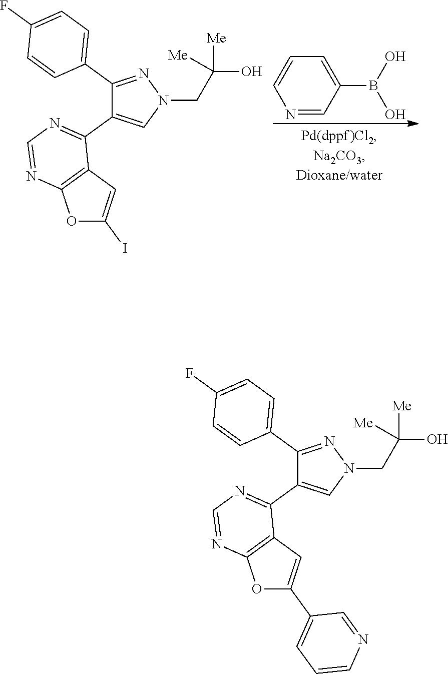

To a stirred solution of 1-[3-(4-fluorophenyl)-4-{6-iodofuro[2,3-d]pyrimidin-4-yl}pyrazol-1-yl]-2-methylpropan-2-ol (Intermediate X, 120 mg, 0.251 mmol, 1 equiv.) and pyridin-3-ylboronic acid (37.01 mg, 0.301 mmol, 1.2 equiv.) in 1,4-dioxane (3.2 mL) and H₂O (0.8 mL) was added Na₂CO₃ (79.78 mg, 0.753 mmol, 3 equiv.) and Pd(dppf)Cl₂·CH₂Cl₂ (20.44 mg, 0.025 mmol, 0.1 equiv.) at room temperature. The resulting mixture was stirred for 3 h at 80° C. under nitrogen then cooled down to room temperature. The reaction was quenched by the addition of water (10 mL) and extracted with EtOAc (3×10 mL). The combined organic layers were washed with brine (3×10 mL) and dried over anhydrous Na₂SO₄. After filtration, the filtrate was concentrated under reduced pressure. The crude product (145 mg) was purified by Prep-HPLC with the following conditions (Column: XSelect CSH Prep C18 OBD Column, 19×250 mm, 5 μm; Mobile Phase A: water (10 mmol/L NH₄HCO₃), Mobile Phase B: ACN; Flow rate: 25 mL/min; Gradient: 46% B to 46% B in 8 min, 46% B; Wavelength: 254 nm; RT1 (min): 8.7) to afford Example 255 (57.5 mg, 53%) as a white solid. m/z (ESI, +ve ion)=430.25 [M+H]⁺. ¹H NMR (400 MHz, DMSO-d₆) δ 9.12 (d, J=2.4 Hz, 1H), 8.82 (s, 1H), 8.70-8.64 (m, 1H), 8.53 (s, 1H), 8.30-8.24 (m, 1H), 7.65-7.57 (m, 3H), 7.51 (s, 1H), 7.25-7.15 (m, 2H), 4.85 (s, 1H), 4.21 (s, 2H), 1.21 (s, 6H).

### Example 256. 4-{3-(4-fluorophenyl)-1-[(1s,3s)-3-(methanesulfonyl)cyclobutyl]-1H-pyrazol-4-yl}-6-(1-methyl-1H-pyrazol-4-yl)furo[2,3-d]pyrimidine

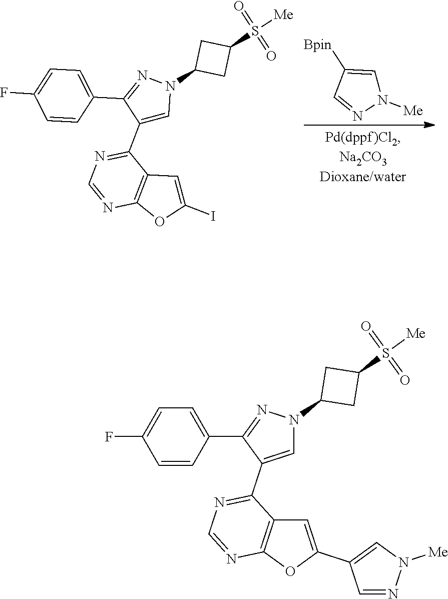

To a stirred mixture of 3-(4-fluorophenyl)-4-{6-iodofuro[2,3-d]pyrimidin-4-yl}-1-[(cis)-3-methanesulfonylcyclobutyl]pyrazole (Intermediate Y, 80 mg, 0.149 mmol, 1 equiv.) and 1-methyl-4-(4,4,5,5-tetramethyl-1,3,2-dioxaborolan-2-yl)pyrazole (46.38 mg, 0.223 mmol, 1.5 equiv.) in dioxane (1 mL) and water (0.2 mL) at room temperature under nitrogen was added Pd(dppf)Cl₂·CH₂Cl₂ (24.21 mg, 0.030 mmol, 0.2 equiv.) and Na₂CO₃ (47.25 mg, 0.447 mmol, 3 equiv.). The resulting mixture was stirred for 3 h at 80° C. under nitrogen then quenched with water (20 mL) at room temperature. The resulting mixture was extracted with EtOAc (3×10 mL). The combined organic layers were washed with brine (1×20 mL) and dried over anhydrous Na₂SO₄. After filtration, the filtrate was concentrated under reduced pressure. The residue was purified by Prep-HPLC with the following conditions (Column: XSelect CSH Prep C18 OBD Column, 19×250 mm, 5 μm; Mobile Phase A: water (0.05% FA), Mobile Phase B: MeOH-HPLC; Flow rate: 25 mL/min; Gradient: 63% B to 63% B in 8 min, 63% B; Wavelength: 220 nm; RT1 (min): 7.3 to afford Example 256 (19.1 mg, 26%) as a white solid. m/z (ESI, +ve ion)=493.05 [M+H]⁺. ¹H NMR (400 MHz, DMSO-d₆) δ 8.73 (s, 1H), 8.58 (s, 1H), 8.31 (s, 1H), 7.93 (s, 1H), 7.63-7.52 (m, 2H), 7.24-7.15 (m, 2H), 6.91 (s, 1H), 5.10-5.06 (m, 1H), 3.97 (t, J=8.4 Hz, 1H), 3.93 (s, 3H), 3.00-2.91 (m, 5H), 2.90-2.78 (m, 2H).

### Example 257. 1-{3-(4-fluorophenyl)-4-[6-(pyridin-2-yl)furo[2,3-d]pyrimidin-4-yl]-1H-pyrazol-1-yl}-2-methylpropan-2-ol

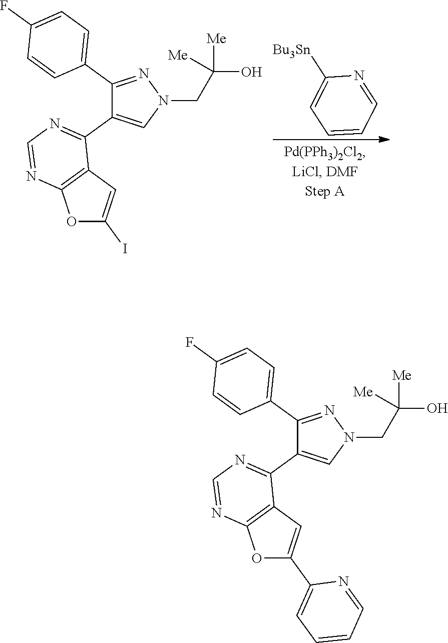

To a stirred solution of 1-[3-(4-fluorophenyl)-4-{6-iodofuro[2,3-d]pyrimidin-4-yl}pyrazol-1-yl]-2-methylpropan-2-ol (Intermediate X, 130 mg, 0.272 mmol, 1 equiv.) and 2-(tributylstannyl)pyridine (250.17 mg, 0.680 mmol, 2.5 equiv.) in DMF (3 mL) was added LiCl (11.52 mg, 0.272 mmol, 1 equiv.) and Pd(PPh₃)₂Cl₂ (19.08 mg, 0.027 mmol, 0.1 equiv.) at room temperature. The resulting mixture was stirred for 5 h at 80° C. under nitrogen then cooled down to room temperature. The reaction was quenched by the addition of water (10 mL) and extracted with EtOAc (3×10 mL). The combined organic layers were washed with brine (3×10 mL) and dried over anhydrous Na₂SO₄. After filtration, the filtrate was concentrated under reduced pressure. The crude product (200 mg) was purified by Prep-HPLC with the following conditions (Column: XSelect CSH Prep C18 OBD Column, 19×250 mm, 5 μm; Mobile Phase A: water (10 mmol/L NH₄HCO₃), Mobile Phase B: MeOH-HPLC; Flow rate: 25 mL/min; Gradient: 70% B to 72% B in 8 min, 72% B; Wavelength: 254 nm; RT1 (min): 8) to afford Example 257 (35.4 mg, 30%) as a white solid. m/z (ESI, +ve ion)=430.15 [M+H]⁺. ¹H NMR (400 MHz, DMSO-d₆) δ 8.96 (s, 1H), 8.70 (d, J=4.8 Hz, 1H), 8.28 (s, 1H), 8.04 (d, J=7.6 Hz, 1H), 7.98 (t, J=8.0 Hz, 1H), 7.68-7.60 (m, 2H), 7.44 (t, J=6.4 Hz, 2H), 7.08 (t, J=8.8 Hz, 2H), 4.25 (s, 2H), 1.33 (s, 6H).

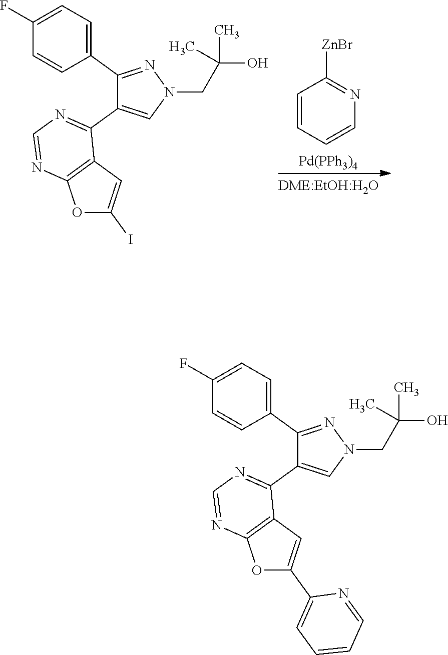

2-Pyridylzinc bromide solution (0.5 M in THF, 261 μL, 131 μmol, 1.25 equiv.) was added to a degassed mixture of 1-[3-(4-Fluorophenyl)-4-{6-iodofuro[2,3-d]pyrimidin-4-yl}pyrazol-1-yl]-2-methylpropan-2-ol (Intermediate X, 50.0 mg, 105 μmol, 1.0 equiv.) and tetrakis(triphenylphosphine)palladium (0) (1.23 mg, 1.05 μmol, 0.01 equiv.) The mixture was stirred at room temperature for 16 h. The mixture was diluted with saturated aqueous NH₄C₁, the aqueous layer was extracted with EtOAc (3×10 mL). The combined organic layers were washed with saturated aqueous NaHCO₃, brine, then dried (Na₂SO₄), filtered and concentrated under reduced pressure. The crude was purified by column chromatography (MeOH in DCM, 0-10%, a gradient elution), to provide the title compound (27.7 mg, 62%) as a solid.

### Example 258.1-{4-[6-(1,5-dimethyl-1H-pyrazol-4-yl)furo[2,3-d]pyrimidin-4-yl]-3-(4-fluorophenyl)-1H-pyrazol-1-yl}-2-methylpropan-2-ol

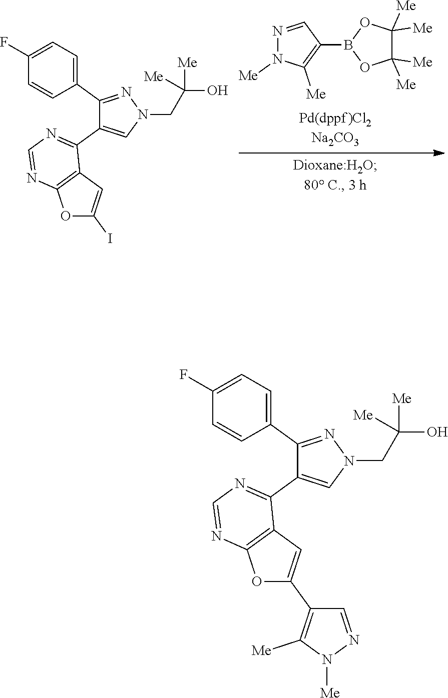 [SMILES: CC(C)(O)Cn1cc(-c2ncnc3oc(I)cc23)c(-c2ccc(F)cc2)n1 ; Cc1c(B2OC(C)(C)C(C)(C)O2)cnn1C ; Cc1c(-c2cc3c(-c4cn(CC(C)(C)O)nc4-c4ccc(F)cc4)ncnc3o2)cnn1C]

Sodium carbonate (35.5 mg, 0.33 mmol, 2.00 equiv), 1-[3-(4-fluorophenyl)-4-(6-iodofuro[2,3-d]pyrimidin-4-yl)pyrazol-1-yl]-2-methyl-propan-2-ol (Intermediate X, 80 mg, 0.17 mmol, 1.00 equiv) and 1,5-dimethyl-4-(4,4,5,5-tetramethyl-1,3,2-dioxaborolan-2-yl)pyrazole (37.2 mg, 0.17 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (13.7 mg, 0.02 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by Column: Gemini 10 μm C18 110 Å LC Column, 250×30 mm; Mobile Phase A: water (10 mmol/L NH₄HCO₃), Mobile Phase B: ACN; Flow rate: 40 mL/min; Gradient: 35% B to 60% B in 22 min; Wavelength: 254 nm; RT1 (min): 12.0 to afford Example 258 (31 mg, 42%) as a white solid. m/z (ESI+ve ion)=447.20 [M+H]⁺.

¹H NMR (400 MHz, DMSO) δ 8.74 (s, 1H), 8.40 (s, 1H), 7.79 (s, 1H), 7.55 (s, 2H), 7.19 (s, 2H), 6.42 (s, 1H), 4.86 (s, 1H), 4.18 (s, 2H), 3.81 (s, 3H), 2.38 (s, 3H), 1.18 (s, 6H).

### Example 259. 4-{3-(4-fluorophenyl)-1-[(1s,3s)-3-(methanesulfonyl)cyclobutyl]-1H-pyrazol-4-yl}-6-(1-methyl-1H-imidazol-4-yl)furo[2,3-d]pyrimidine

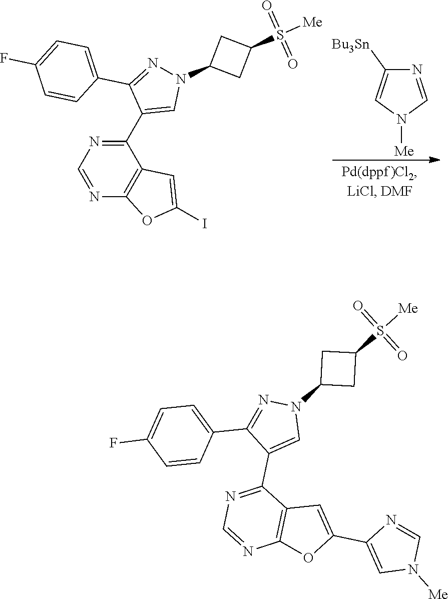

To a stirred solution of 3-(4-fluorophenyl)-4-{6-iodofuro[2,3-d]pyrimidin-4-yl}-1-[(cis)-3-methanesulfonylcyclobutyl]pyrazole (Intermediate Y, 90 mg, 0.167 mmol, 1 equiv.) and 1-methyl-4-(tributylstannyl)imidazole (93.08 mg, 0.251 mmol, 1.5 equiv.) in DMF (2 mL) were added Pd(dppf)Cl₂·CH₂Cl₂ (27.24 mg, 0.033 mmol, 0.2 equiv.) and LiCl (21.26 mg, 0.501 mmol, 3 equiv.) at room temperature. The resulting mixture was stirred for 2 h at 80° C. under nitrogen then cooled down to room temperature. The reaction was quenched by the addition of water (10 mL). The resulting mixture was extracted with EtOAc (3×10 mL). The combined organic layers were washed with brine (3×10 mL) and dried over anhydrous Na₂SO₄. After filtration, the filtrate was concentrated under reduced pressure. The crude product (60 mg) was purified by Prep-HPLC with the following conditions (Column: XBridge Prep OBD C18 Column, 30×150 mm, 5 μm; Mobile Phase A: water (10 mmol/L NH₄HCO₃), Mobile Phase B: ACN; Flow rate: 60 mL/min; Gradient: 26% B to 36% B in 8 min, 36% B; Wavelength: 254 nm; RT1 (min): 7 to afford Example 259 (35.8 mg, 43%) as a white solid. m/z (ESI, +ve ion)=493.10 [M+H]⁺. ¹H-NMR (400 MHz, DMSO-d₆) δ 8.72 (s, 1H), 8.65 (s, 1H), 7.81-7.77 (m, 2H), 7.62-7.56 (m, 2H), 7.20 (t, J=8.8 Hz, 2H), 6.85 (s, 1H), 5.07 (t, J=8.4 Hz, 1H), 3.99-3.91 (m, 1H), 3.75 (s, 3H), 3.00-3.92 (m, 5H), 2.84-2.79 (m, 2H).

### Example 260. 1-[5-{4-[3-(4-fluorophenyl)-1-(2-hydroxy-2-methylpropyl)-1H-pyrazol-4-yl]furo[2,3-d]pyrimidin-6-yl}-3,4-dihydropyridin-1 (2H)-yl]ethan-1-one

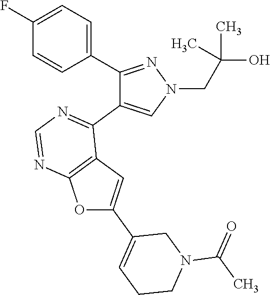 [SMILES: CC(=O)N1CCC=C(c2cc3c(-c4cn(CC(C)(C)O)nc4-c4ccc(F)cc4)ncnc3o2)C1]

### Step 1. tert-Butyl 3-(4-(3-(4-fluorophenyl)-1-(2-hydroxy-2-methylpropyl)-1H-pyrazol-4-yl)furo[2,3-d]pyrimidin-6-yl)-5,6-dihydropyridine-1 (2H)-carboxylate

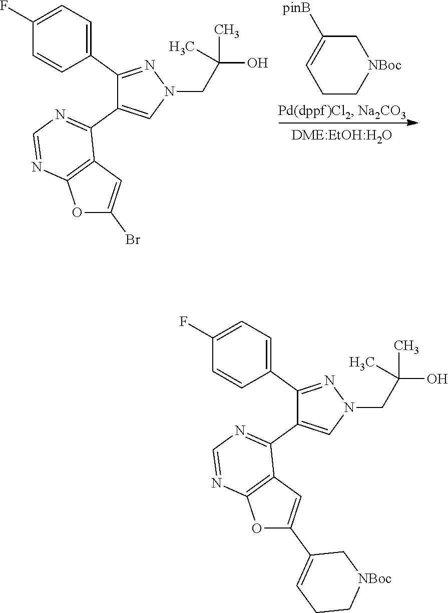

1-(4-(6-Bromofuro[2,3-d]pyrimidin-4-yl)-3-(4-fluorophenyl)-1H-pyrazol-1-yl)-2-methylpropan-2-ol (Product of Step 1 from Example 138, 355 mg, 742 μmol, 1.0 equiv.), tert-butyl 5-(4,4,5,5-tetramethyl-1,3,2-dioxaborolan-2-yl)-3,4-dihydropyridine-1 (2H)-carboxylate (290 mg, 891 μmol, 1.20 equiv.), sodium carbonate (238 mg, 2.23 mmol, 3.0 equiv.) and 1,1′-bis(diphenylphosphino)ferrocene dichloropalladium (II) (57.2 mg, 74.2 μmol, 0.1 equiv.) were dissolved in DME (4.81 mL), EtOH (2.41 mL) and H₂O (1.20 mL). The mixture was degassed with nitrogen and stirred at 85° C. for 16 hours. The mixture was cooled to room temperature, diluted with water and the aqueous layer was extracted with EtOAc (3×20 mL). The combined organic layers were washed with brine and concentrated under reduced pressure. The crude material was purified by column chromatography (EtOAc in heptanes, 30-100%, a gradient elution), to provide the title compound (224 mg, 57%) as a solid. m/z (ESI, +ve ion)=534.3 [M+H]⁺.

### Step 2. 1-(3-(4-Fluorophenyl)-4-(6-(1,2,5,6-tetrahydropyridin-3-yl)furo[2,3-d]pyrimidin-4-yl)-1H-pyrazol-1-yl)-2-methylpropan-2-ol

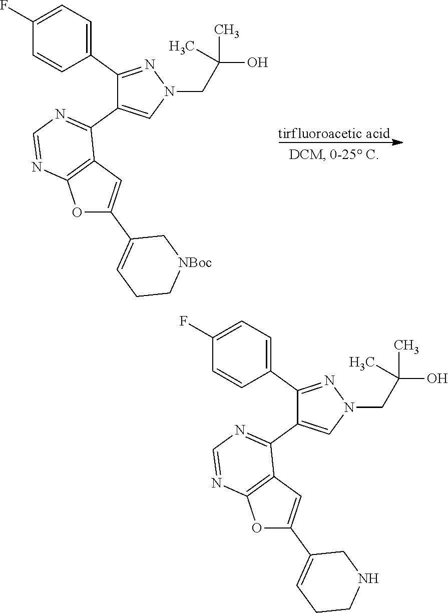

Tert-butyl 3-(4-(3-(4-fluorophenyl)-1-(2-hydroxy-2-methylpropyl)-1H-pyrazol-4-yl)furo[2,3-d]pyrimidin-6-yl)-5,6-dihydropyridine-1 (2H)-carboxylate (224 mg, 420 μmol, 1.0 equiv.) was dissolved in DCM (1.05 mL). The solution was cooled to 0° C., then trifluoroacetic acid (325 μL, 4.20 mmol, 10.0 equiv.) was added. The reaction mixture was stirred at room temperature for 1 hour. The solvent was evaporated, and the pH was adjusted to 7-8 using 15% w/w aqueous NaOH solution. The aqueous phase was extracted with EtOAc (×3), the combined organic layers were washed with brine, dried with Na₂SO₄, filtered, and evaporated to afford the title compound (182 mg, 100%) as a solid. m/z (ESI, +ve ion)=434.2 [M+H]⁺.

### Step 3. 1-[5-{4-[3-(4-fluorophenyl)-1-(2-hydroxy-2-methylpropyl)-1H-pyrazol-4-yl]furo[2,3-d]pyrimidin-6-yl}-3,4-dihydropyridin-1 (2H)-yl]ethan-1-one

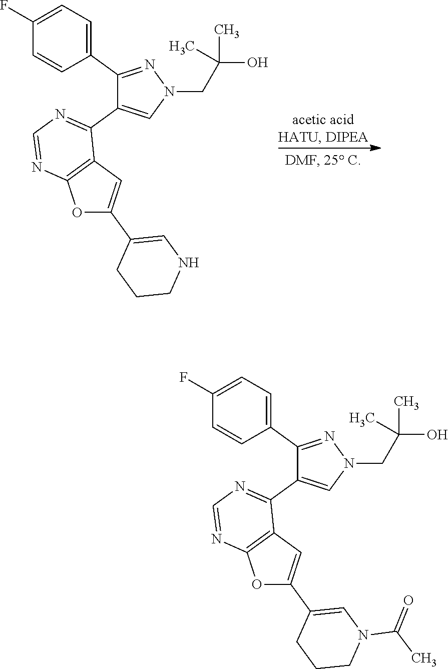 [SMILES: CC(C)(O)Cn1cc(-c2ncnc3oc(C4=CNCCC4)cc23)c(-c2ccc(F)cc2)n1 ; "acetic acidHATU, DIPEA" ; CC(=O)N1C=C(c2cc3c(-c4cn(CC(C)(C)O)nc4-c4ccc(F)cc4)ncnc3o2)CCC1]

Acetic acid (48.0 μL, 830 μmol, 4.0 equiv.) and N,N-diisopropylethylamine (145 μL, 830 μmol, 4.0 equiv.) were added to a solution of 1-(3-(4-fluorophenyl)-4-(6-(1,2,5,6-tetrahydropyridin-3-yl)furo[2,3-d]pyrimidin-4-yl)-1H-pyrazol-1-yl)-2-methylpropan-2-ol (90.0 mg, 208 μmol, 1.0 equiv.) in DMF (1.04 mL) at room temperature. HATU (120 mg, 311 μmol, 1.5 equiv.) was added and the reaction mixture was stirred at room temperature for 1 hour. The crude material was purified by reverse phase chromatography (C18 column, MeCN in water with 10 mM ammonium formate, 5-50%, a gradient elution) to afford the title compound (13.6 mg, 14%) as a solid. ¹H NMR (400 MHz, DMSO-d₆) δ 8.69 (d, J=3.7 Hz, 1H), 8.42 (s, 1H), 7.58-7.53 (m, 3H), 7.19 (t, J=8.5 Hz, 2H), 6.41 (s, 1H), 4.84 (s, 1H), 4.17 (s, 2H), 3.72-3.60 (m, 2H), 2.32 and 2.21 (s, 3H), 2.30-2.22 (m, 2H), 1.97-1.82 (m, 2H), 1.18 (s, 6H). Note: coalescence observed for peaks at 2.32 and 2.21 ppm at 90° C. m/z (ESI, +ve ion)=476.3 [M+H]⁺.

### Example 261. 1-{3-(4-fluorophenyl)-4-[6-(1-methyl-1H-pyrazol-4-yl)furo[2,3-d]pyrimidin-4-yl]-1H-pyrazol-1-yl}-2-methylpropan-2-ol

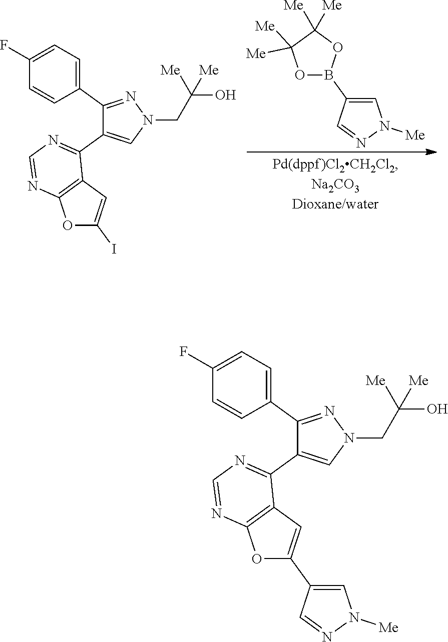 [SMILES: CC(C)(O)Cn1cc(-c2ncnc3oc(I)cc23)c(-c2ccc(F)cc2)n1 ; Cn1cc(B2OC(C)(C)C(C)(C)O2)cn1 ; Cn1cc(-c2cc3c(-c4cn(CC(C)(C)O)nc4-c4ccc(F)cc4)ncnc3o2)cn1]

To a stirred solution of 1-[3-(4-fluorophenyl)-4-{6-iodofuro[2,3-d]pyrimidin-4-yl}pyrazol-1-yl]-2-methylpropan-2-ol (Intermediate X, 100 mg, 0.209 mmol, 1 equiv) and 1-methyl-4-(4,4,5,5-tetramethyl-1,3,2-dioxaborolan-2-yl)pyrazole (52.21 mg, 0.251 mmol, 1.2 equiv) in 1,4-dioxane (0.5 mL) and water (0.1 mL) was added Pd(dppf)Cl₂·CH₂Cl₂ (34.07 mg, 0.042 mmol, 0.2 equiv) and Na₂CO₃ (66.48 mg, 0.627 mmol, 3 equiv) at room temperature under nitrogen atmosphere. The resulting mixture was stirred for 3 h at 80° C. The mixture was allowed to cool down to room temperature. Water (10 mL) was added and the resulting mixture was extracted with EtOAc (3×30 mL). The combined organic layers were washed with brine (20 mL), dried over anhydrous Na₂SO₄, and filtered. The filtrate was concentrated under reduced pressure, and the residue was purified by prep-HPLC with the following conditions (Column: XBridge Prep OBD C18 Column, 30×150 mm, 5 m; Mobile Phase A: water (10 mmol/L NH₄HCO₃), Mobile Phase B: ACN; Flow rate: 60 mL/min; Gradient: 28% B to 38% B in 8 min, 38% B; Wavelength: 254 nm; RT1 (min): 7) to afford the title compound (31.2 mg, 34%) as a white solid. m/z (ESI, +ve ion)=433.15 [M+H]⁺. ¹H NMR (400 MHz, DMSO-d₆) δ 8.72 (s, 1H), 8.42 (s, 1H), 8.31 (s, 1H), 7.92 (s, 1H), 7.60-7.57 (m, 2H), 7.21-7.16 (m, 2H), 6.83 (s, 1H), 4.84 (s, 1H), 4.19 (s, 2H), 3.93 (s, 3H), 1.20 (s, 6H).

### Example 262. 1-{4-[6-(2-chlorophenyl)furo[2,3-d]pyrimidin-4-yl]-3-(4-fluorophenyl)-1H-pyrazol-1-yl}-2-methylpropan-2-ol

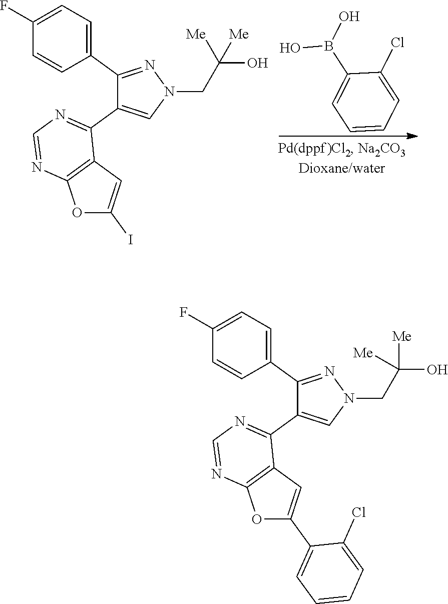

A solution of 1-[3-(4-fluorophenyl)-4-{6-iodofuro[2,3-d]pyrimidin-4-yl}pyrazol-1-yl]-2-methylpropan-2-ol (Intermediate X, 100 mg, 0.209 mmol, 1 equiv), 2-chlorophenylboronic acid (39.23 mg, 0.251 mmol, 1.2 equiv), Na₂CO₃ (66.48 mg, 0.627 mmol, 3 equiv) and Pd(dppf)Cl₂ (30.60 mg, 0.042 mmol, 0.2 equiv) in dioxane (4.00 mL) and water (1.00 mL) was stirred for 2 h at 80° C. under nitrogen atmosphere. The resulting mixture was diluted with water (30 mL). The mixture was extracted with DCM (3×50 mL). The combined organic layers were washed with brine (30 mL) and dried over anhydrous Na₂SO₄. After filtration, the filtrate was concentrated under reduced pressure. The crude product was purified by prep-HPLC with the following conditions Column: XBridge Prep Phenyl OBD Column, 19×150 mm, 5 m; Mobile Phase A: water (10 mmol/L NH₄HCO₃), Mobile Phase B: ACN; Flow rate: 25 mL/min; Gradient: 57% B to 62% B in 8 min, 62% B; Wavelength: 254 nm; RT1 (min): 7.5 to afford the title compound (44.7 mg, 46%) as an off-white solid. m/z (ESI, +ve ion)=463.05 [M+H]⁺. ¹H NMR (400 MHz, Chloroform-d) δ 8.98 (s, 1H), 8.16 (s, 1H), 8.07-8.00 (m, 1H), 7.60-7.51 (m, 2H), 7.49-7.29 (m, 3H), 7.13-7.04 (m, 2H), 6.78 (s, 1H), 4.24 (s, 2H), 1.33 (s, 6H).

### Example 263. 4-[1-(3,3-difluoropropyl)-3-(4-fluorophenyl)-1H-pyrazol-4-yl]-6-(1-methyl-1H-imidazol-4-yl)furo[2,3-d]pyrimidine

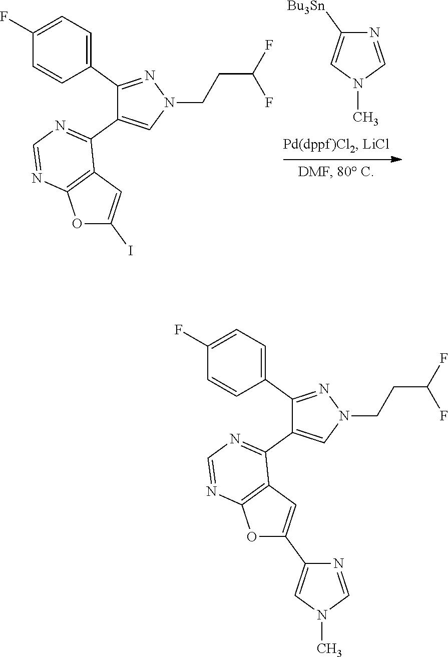

1,1′-Bis(diphenylphosphino)ferrocene dichloropalladium (II) (15.9 mg, 20.7 μmol, 0.1 equiv.) was added to a degassed mixture of 4-(1-(3,3-difluoropropyl)-3-(4-fluorophenyl)-1H-pyrazol-4-yl)-6-iodofuro[2,3-d]pyrimidine (Intermediate Z, 100 mg, 207 μmol, 1 equiv.), lithium chloride (8.76 mg, 207 μmol, 1.0 equiv.) and N-methyl-4-(tributylstannyl)imidazole (94.4 μL, 258 μmol, 1.25 equiv.) in DMF (2.37 mL). The mixture was stirred for 2 h at 80° C. then cooled to room temperature, diluted with water (20 mL), and extracted with EtOAc (3×10 mL). The combined organic layers were washed with brine, then dried (Na₂SO₄), filtered and concentrated under reduced pressure. The crude product was purified by column chromatography (acetone in hexanes, 0-80%, a gradient elution) twice to provide the title compound (44.4 mg, 49%) as a solid. ¹H NMR (400 MHz, DMSO-d₆) δ 8.69 (s, 1H), 8.69 (s, 1H), 7.80 (s, 1H), 7.80 (s, 1H), 7.59 (dd, J=8.5, 5.7 Hz, 2H), 7.18 (t, J=8.8 Hz, 2H), 6.94 (s, 1H), 6.27 (tt, J=56.3, 4.3 Hz, 1H), 4.45 (t, J=7.1 Hz, 2H), 3.74 (s, 3H), 2.61-2.50 (m, 2H). m/z (ESI, +ve ion)=439.10 [M+H]⁺.

### Example 264. 4-{1-[(1s,3s)-3-fluorocyclobutyl]-3-(4-fluorophenyl)-1H-pyrazol-4-yl}-6-(1-methyl-1H-imidazol-4-yl)furo[2,3-d]pyrimidine

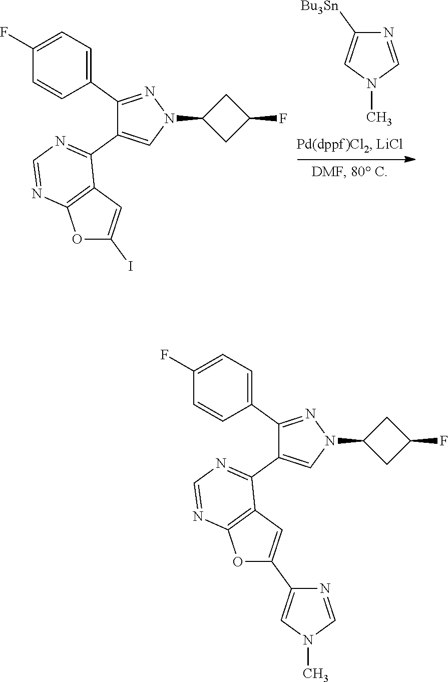

1,1′-Bis(diphenylphosphino)ferrocene dichloropalladium (II) (16.1 mg, 20.9 μmol, 0.1 equiv.) was added to a degassed mixture of 4-(1-((1s,3s)-3-fluorocyclobutyl)-3-(4-fluorophenyl)-1H-pyrazol-4-yl)-6-iodofuro[2,3-d]pyrimidine (Intermediate AA, 100 mg, 209 μmol, 1.0 equiv.), N-methyl-4-(tributylstannyl)imidazole (95.6 μL, 261 μmol, 1.25 equiv.) and lithium chloride (8.87 mg, 209 μmol, 1.0 equiv.) in DMF (2.40 mL). The mixture was stirred for 2 h at 80° C. then cooled to room temperature and diluted with water (10 mL) and EtOAc (10 mL). The mixture was extracted with EtOAc (3×15 mL), washed with water and brine then dried (Na₂SO₄), filtered and concentrated under reduced pressure. The crude product was purified by reverse phase chromatography (C18 column, MeCN in water with 10 mM ammonium bicarbonate, 5-60%, a gradient elution) followed by reverse phase chromatography (C18 column, MeCN in water with 10 mM ammonium bicarbonate, 10-50%, a gradient elution) to provide the title compound (30.3 mg, 34%) as a solid. ¹H NMR (400 MHz, DMSO-d₆) δ 8.70 (s, 1H), 8.70 (s, 1H), 7.79 (s, 1H), 7.79 (s, 1H), 7.64-7.54 (m, 2H), 7.19 (t, J=8.7 Hz, 2H), 6.92 (s, 1H), 5.19-4.93 (m, 1H), 4.64 (p, J=7.9 Hz, 1H), 3.73 (s, 3H), 3.08-2.76 (m, 4H). m/z (ESI, +ve ion)=433.2 [M+H]⁺.

### Example 265. 3-{3-(4-fluorophenyl)-4-[6-(1-methyl-1H-imidazol-4-yl)furo[2,3-d]pyrimidin-4-yl]-1H-pyrazol-1-yl}propanenitrile

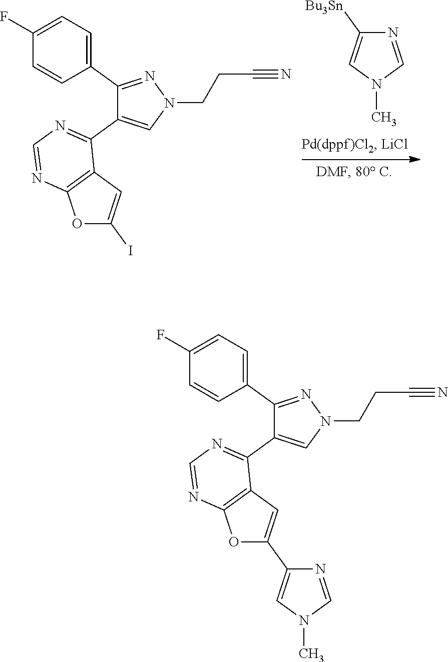

1,1′-Bis(diphenylphosphino)ferrocene dichloropalladium (II) (21.8 mg, 28.3 μmol, 0.1 equiv.) was added to a degassed mixture of 3-(3-(4-fluorophenyl)-4-(6-iodofuro[2,3-d]pyrimidin-4-yl)-1H-pyrazol-1-yl)propanenitrile (Intermediate AB, 140 mg, 283 μmol, 1.0 equiv.), lithium chloride (12.0 mg, 283 μmol, 1.0 equiv.) and N-methyl-4-(tributylstannyl)imidazole (129 μL, 354 umol, 1.25 equiv.) in DMF (3.25 mL). The mixture was stirred for 2 h at 80° C. The reaction mixture was cooled to room temperature, diluted with water (20 mL), and extracted with EtOAc (3×10 mL). The combined organic layers were washed with brine, dried (MgSO₄), filtered and concentrated under reduced pressure. The crude material was purified by reverse phase (C18 column, MeCN in water with 10 mM ammonium formate, 30-70%, a gradient elution) to provide the title compound (16.0 mg, 13%) as a solid. ¹H NMR (400 MHz, DMSO-d₆) δ 8.72 (s, 1H), 8.71 (s, 1H), 7.80 (s, 2H), 7.62-7.59 (m, 2H), 7.21-7.17 (m, 2H), 6.92 (s, 1H), 4.57 (t, J=6.4 Hz, 2H), 3.74 (s, 3H), 3.23 (t, J=6.4 Hz, 2H). m/z (ESI, +ve ion)=414.2 [M+H]⁺.

### Example 266. 2,2-difluoro-3-{3-(4-fluorophenyl)-4-[6-(1-methyl-1H-imidazol-4-yl)furo[2,3-d]pyrimidin-4-yl]-1H-pyrazol-1-yl}propan-1-ol

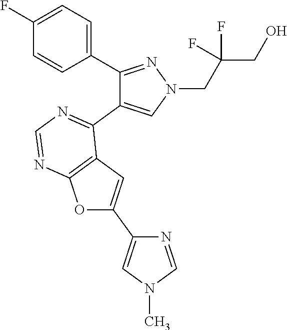 [SMILES: Cn1cnc(-c2cc3c(-c4cn(CC(F)(F)CO)nc4-c4ccc(F)cc4)ncnc3o2)c1]

### Step A. 4-(1-(3-((di-tert-butyl(phenyl)silyl)oxy)-2,2-difluoropropyl)-3-(4-fluorophenyl)-1H-pyrazol-4-yl)-6-(1-methyl-1H-imidazol-4-yl)furo[2,3-d]pyrimidine

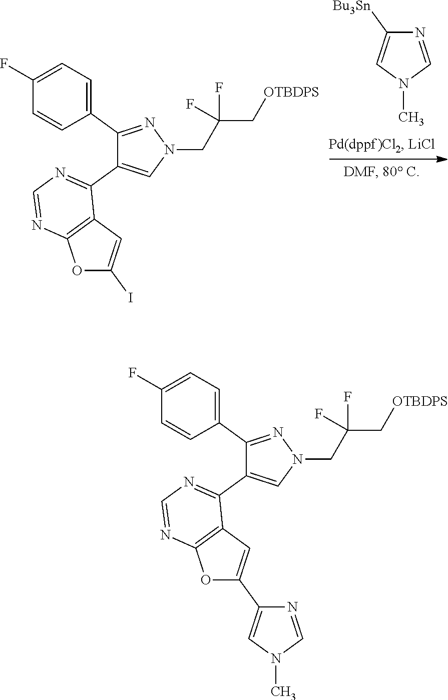

1,1′-Bis(diphenylphosphino)ferrocene dichloropalladium (II) (15.6 mg, 20.3 μmol, 0.1 equiv.) was added to a degassed mixture of 4-(1-(3-((tert-butyldiphenylsilyl)oxy)-2,2-difluoropropyl)-3-(4-fluorophenyl)-1H-pyrazol-4-yl)-6-iodofuro[2,3-d]pyrimidine (Intermediate AC, 150 mg, 203 μmol, 1.0 equiv.), lithium chloride (8.62 mg, 203 μmol, 1.0 equiv.) and N-methyl-4-(tributylstannyl)imidazole (92.8 μL, 254 μmol, 1.25 equiv.) in DMF (2.33 mL). The mixture was stirred for 2 h at 80° C. then cool to room temperature, diluted with water (20 mL), and extracted with EtOAc (3×10 mL). The combined organic layers were washed with water, brine, then dried (Na₂SO₄), filtered and concentrated under reduced pressure. The crude material was purified by column chromatography (acetone in hexanes, 0-70%, a gradient elution) twice to provide the title compound (100 mg, 64%) as a solid. ¹H NMR (400 MHz, CDCl₃) δ 8.82 (s, 1H), 8.08 (s, 1H), 7.67 (d, J=7.1 Hz, 4H), 7.56 (dd, J=8.5, 5.6 Hz, 2H), 7.49 (s, 1H), 7.44-7.37 (m, 3H), 7.31 (t, J=7.4 Hz, 4H), 7.02 (t, J=8.7 Hz, 2H), 6.57 (s, 1H), 4.79 (t, J=12.7 Hz, 2H), 3.82 (t, J=11.9 Hz, 2H), 3.76 (s, 3H), 1.10 (s, 9H). m/z (ESI, +ve ion)=693.4 [M+H]⁺.

### Step B. 2,2-difluoro-3-{3-(4-fluorophenyl)-4-[6-(1-methyl-1H-imidazol-4-yl)furo[2,3-d]pyrimidin-4-yl]-1H-pyrazol-1-yl}propan-1-ol

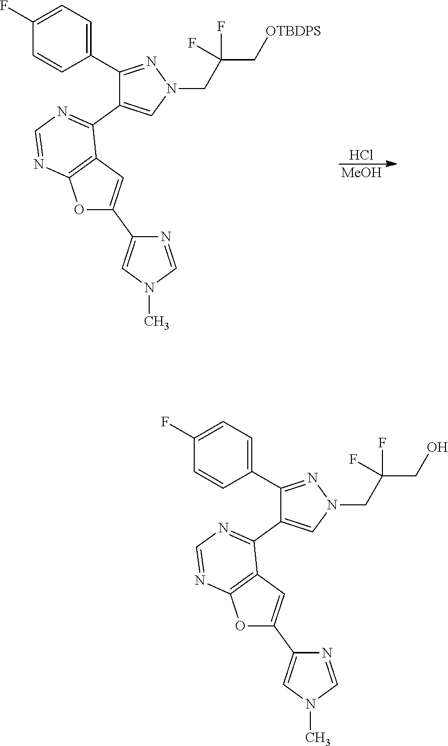

Hydrochloric acid (962 μL, 2.89 mmol, 25 equiv.) was added to a mixture of 4-(1-(3-((tert-butyldiphenylsilyl)oxy)-2,2-difluoropropyl)-3-(4-fluorophenyl)-1H-pyrazol-4-yl)-6-(1-methyl-1H-imidazol-4-yl)furo[2,3-d]pyrimidine (80.0 mg, 115 μmol, 1.0 equiv.) in MeOH (400 μL). The mixture was stirred for 6 h then hydrochloric acid (962 μL, 2.89 mmol, 25 equiv.) was added. The mixture was stirred for 15 h then diluted with saturated aqueous NaHCO₃ (20 mL) and extracted with EtOAc (3×10 mL). The combined organic layers were washed with brine, then dried (Na₂SO₄), filtered and concentrated under reduced pressure. The crude material was purified by reverse phase chromatography (C18 column, MeCN in water with 10 mM ammonium formate, 10-60%, a gradient elution) to provide the title compound (34.6 mg, 66%) as a solid. ¹H NMR (400 MHz, DMSO-d₆) δ 8.73 (s, 1H), 8.64 (s, 1H), 7.80 (s, 2H), 7.59 (dd, J=8.3, 5.8 Hz, 2H), 7.19 (t, J=8.8 Hz, 2H), 6.81 (s, 1H), 5.80 (t, J=6.1 Hz, 1H), 4.86 (t, J=14.1 Hz, 2H), 3.82-3.67 (m, 2H), 3.74 (s, 3H). m/z (ESI, +ve ion)=454.14 [M+H]⁺.

### Example 267. 1-[4-{6-[5-(difluoromethyl)-1-methyl-1H-pyrazol-4-yl]furo[2,3-d]pyrimidin-4-yl}-3-(4-fluorophenyl)-1H-pyrazol-1-yl]-2-methylpropan-2-ol

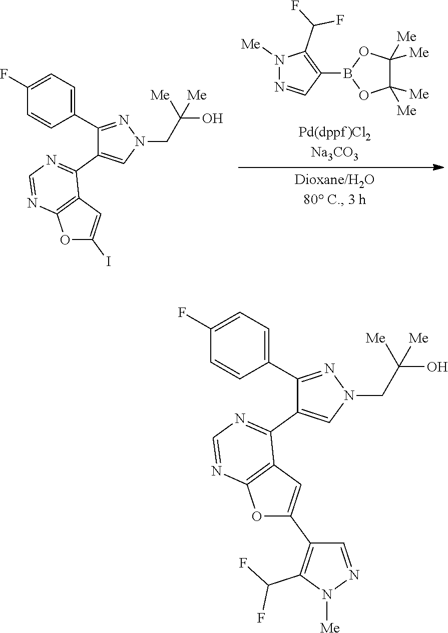 [SMILES: CC(C)(O)Cn1cc(-c2ncnc3oc(I)cc23)c(-c2ccc(F)cc2)n1 ; Cn1ncc(B2OC(C)(C)C(C)(C)O2)c1C(F)F ; Cn1ncc(-c2cc3c(-c4cn(CC(C)(C)O)nc4-c4ccc(F)cc4)ncnc3o2)c1C(F)F]

Sodium carbonate (44.3 mg, 0.42 mmol, 2.00 equiv), 1-[3-(4-fluorophenyl)-4-{6-iodofuro[2,3-d]pyrimidin-4-yl}pyrazol-1-yl]-2-methylpropan-2-ol (Intermediate X, 100 mg, 0.21 mmol, 1.00 equiv) and 5-(difluoromethyl)-1-methyl-4-(4,4,5,5-tetramethyl-1,3,2-dioxaborolan-2-yl)pyrazole (54.0 mg, 0.21 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (17.1 mg, 0.02 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by reverse phase chromatography (Column: Gemini 10 μm C18 110 Å LC Column, 250×50 mm; Mobile Phase A: water (10 mmol/L NH₄HCO₃), Mobile Phase B: ACN; Flow rate: 120 mL/min; Gradient: 20% B to 65% B in 22 min; Wavelength: 254 nm; RT1 (min): 19.5) to provide the title compound (46 mg, 46%) as a white solid. m/z (ESI+ve ion)=483.20 [M+H]⁺. ¹H NMR (400 MHz, DMSO) δ 8.81 (s, 1H), 8.41 (s, 1H), 8.03 (s, 1H), 7.56 (d, J=16.4 Hz, 2H), 7.39 (d, J=51.5 Hz, 1H), 7.18 (t, J=9.4 Hz, 2H), 6.78 (s, 1H), 4.85 (s, 1H), 4.18 (s, 2H), 4.04 (s, 3H), 1.18 (s, 6H).

### Example 268. 3-{3-(4-fluorophenyl)-4-[6-(pyridin-3-yl)furo[2,3-d]pyrimidin-4-yl]-1H-pyrazol-1-yl}propanenitrile

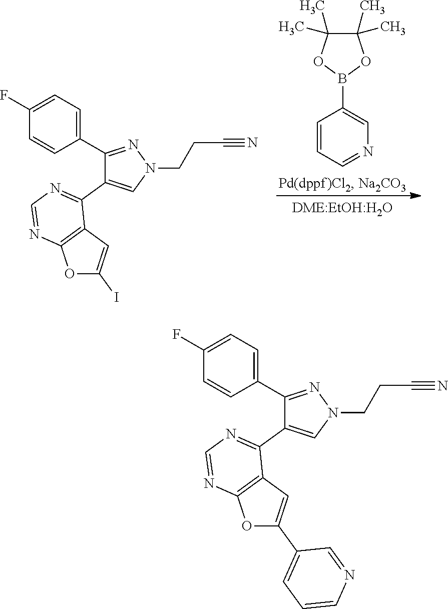

1,1′-Bis(diphenylphosphino)ferrocene dichloropalladium (II) (8.39 mg, 10.9 μmol, 0.05 equiv.) was added to a degassed mixture of 3-(4,4,5,5-tetramethyl-1,3,2-dioxaborolane-2-yl)pyridine (55.2 mg, 261 μmol, 1.2 equiv.), 3-(3-(4-fluorophenyl)-4-(6-iodofuro[2,3-d]pyrimidin-4-yl)-1H-pyrazol-1-yl)propanenitrile (Intermediate AB, 100 mg, 218 μmol, 1.0 equiv.) and sodium carbonate (69.9 mg, 653 μmol, 3.0 equiv.) in DME (1.41 mL), EtOH (706 μL) and H₂O (353 μL). The mixture was stirred for 1.5 h at 90° C. under microwave. The mixture was diluted with water (20 mL). The aqueous layer was extracted with EtOAc (3×20 mL). The combined organic layers were washed with brine, then dried (MgSO₄), filtered and concentrated under reduced pressure. The crude material was purified by column chromatography (methanol in DCM, 1-10%, a gradient elution) then by reverse phase chromatography (C18 column, MeCN in water with 10 mM ammonium formate, 10-100%, a gradient elution), to provide the title compound (41.0 mg, 45%) as a solid. ¹HNMR (400 MHz, DMSO-d6) δ 9.08 (d, J=1.7 Hz, 1H), 8.79 (s, 1H), 8.69 (s, 1H), 8.63 (dd, J=4.8, 1.6 Hz, 1H), 8.25-8.21 (m, 1H), 7.62-7.54 (m, 3H), 7.53 (s, 1H), 7.20-7.14 (m, 2H), 4.54 (t, J=6.4 Hz, 2H), 3.22 (t, J=6.4 Hz, 2H). m/z (ESI, +ve ion)=411.2 [M+H]⁺.

### Example 269. 3-{3-(4-fluorophenyl)-4-[6-(pyridin-2-yl)furo[2,3-d]pyrimidin-4-yl]-1H-pyrazol-1-yl}propanenitrile

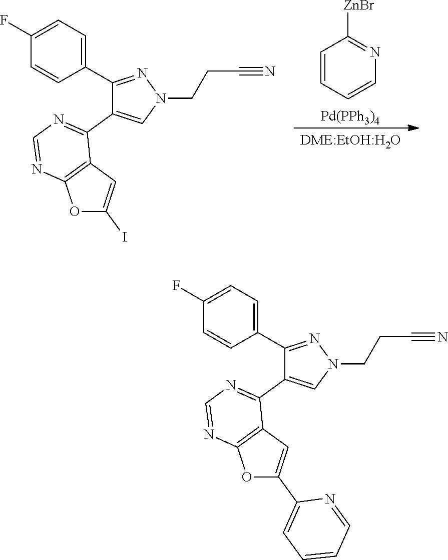

2-Pyridylzinc bromide solution (0.5 M in THF, 544 μL, 272 μmol, 1.25 equiv.) was added to a degassed mixture of 3-(3-(4-fluorophenyl)-4-(6-iodofuro[2,3-d]pyrimidin-4-yl)-1H-pyrazol-1-yl)propanenitrile (Intermediate AB, 100 mg, 218 μmol) and tetrakis(triphenylphosphine)palladium (0) (2.57 mg, 2.18 μmol, 0.01 equiv.). The mixture was stirred for 18 h at 25° C. The crude material was directly purified by column chromatography (methanol in DCM, 1-10%, a gradient elution) and then by reverse phase chromatography (C18 column, MeCN in water with 10 mM ammonium formate, 10-90%, a gradient elution) to provide the title compound (38.0 mg, 41%) as a solid. ¹H NMR (400 MHz, DMSO-d₆) δ 8.79 (s, 2H), 8.70-8.68 (m, 1H), 8.01-7.94 (m, 2H), 7.62-7.58 (m, 2H), 7.57 (s, 1H), 7.48-7.45 (m, 1H), 7.20-7.13 (m, 2H), 4.55 (t, J=6.4 Hz, 2H), 3.21 (t, J=6.4 Hz, 2H). m/z (ESI, +ve ion)=411.2 [M+H]⁺.

### Example 270. (1r,3r)-3-{3-(4-fluorophenyl)-4-[6-(1-methyl-1H-imidazol-4-yl)furo[2,3-d]pyrimidin-4-yl]-1H-pyrazol-1-yl}cyclobutane-1-carbonitrile

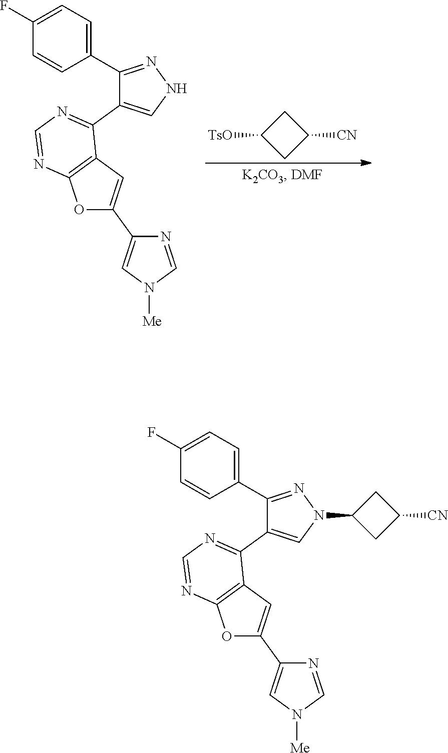 [SMILES: Cn1cnc(-c2cc3c(-c4c[nH]nc4-c4ccc(F)cc4)ncnc3o2)c1 ; Cc1ccc(S(=O)(=O)O[C@H]2C[C@@H](C#N)C2)cc1 ; Cn1cnc(-c2cc3c(-c4cn([C@H]5C[C@H](C#N)C5)nc4-c4ccc(F)cc4)ncnc3o2)c1]

To a stirred solution of 3-(4-fluorophenyl)-4-[6-(1-methylimidazol-4-yl)furo[2,3-d]pyrimidin-4-yl]-1H-pyrazole (Intermediate AE, 100 mg, 0.278 mmol, 1 equiv) and (cis)-3-cyanocyclobutyl 4-methylbenzenesulfonate (Intermediate AD, 104.61 mg, 0.417 mmol, 1.5 equiv) in DMF (3 mL) was added K₂CO₃ (115.06 mg, 0.834 mmol, 3 equiv) at room temperature. The resulting mixture was stirred for 16 h at 80° C. The mixture was allowed to cool to room temperature. The resulting mixture was diluted with water (20 mL). The mixture was extracted with EtOAc (3×20 mL). The combined organic layers were washed with brine (3×10 mL) and dried over anhydrous Na₂SO₄. After filtration, the filtrate was concentrated under reduced pressure. The residue was purified by prep-TLC (PE:EtOAc=1:1) to afford the crude product (120 mg). It was purified by prep-HPLC with the following conditions (Column: XBridge Prep OBD C18 Column, 30×150 mm, 5 m; Mobile Phase A: water (10 mmol/L NH₄HCO₃), Mobile Phase B: ACN; Flow rate: 60 mL/min; Gradient: 35% B to 45% B in 10 min, 45% B; Wavelength: 220 nm; RT1 (min): 7.50-8.28) to afford the title compound (37.3 mg, 31%) as a white solid. m/z (ESI, +ve ion)=440.10 [M+H]⁺. ¹H NMR (400 MHz, DMSO-d₆) δ 8.70 (d, J=2.8 Hz, 2H), 7.78 (d, J=4.8 Hz, 2H), 7.66-7.56 (m, 2H), 7.19 (d, J=8.8 Hz, 2H), 6.92 (s, 1H), 5.43-5.31 (m, 1H), 3.74 (s, 3H), 3.63-3.51 (m, 1H), 3.12-3.00 (m, 2H), 2.92-2.81 (m, 2H).

### Example 271. 1-{3-(4-fluorophenyl)-4-[6-(2-methoxypyridin-3-yl)furo[2,3-d]pyrimidin-4-yl]-1H-pyrazol-1-yl}-2-methylpropan-2-ol

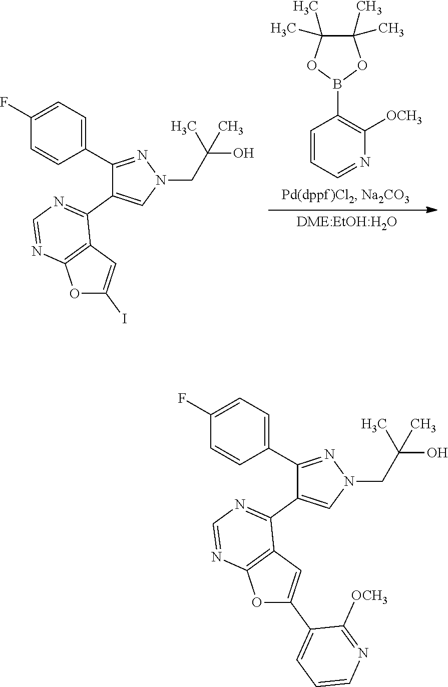 [SMILES: CC(C)(O)Cn1cc(-c2ncnc3oc(I)cc23)c(-c2ccc(F)cc2)n1 ; COc1ncccc1B1OC(C)(C)C(C)(C)O1 ; COc1ncccc1-c1cc2c(-c3cn(CC(C)(C)O)nc3-c3ccc(F)cc3)ncnc2o1]

1,1-Bis(diphenylphosphino)ferrocene dichloropalladium (II) (16.1 mg, 20.9 μmol, 0.1 equiv.) was added to a degassed mixture of 1-[3-(4-Fluorophenyl)-4-{6-iodofuro[2,3-d]pyrimidin-4-yl}pyrazol-1-yl]-2-methylpropan-2-ol (Intermediate X, 100 mg, 209 μmol, 1.00 equiv.), 2-methoxy-3-(4,4,5,5-tetramethyl-1,3,2-dioxaborolan-2-yl)pyridine (51.6 mg, 220 μmol, 1.05 equiv.), and sodium carbonate (67.2 mg, 627 μmol, 3 equiv.) in a mixture of DME (851 μL), EtOH (681 μL) and H₂O (511 μL). The mixture was stirred at 85° C. for 16 h then cooled to room temperature and filtered through Celite® and rinsed with EtOAc. The solvents were removed under reduced pressure. The residue was diluted with saturated aqueous NH₄Cl (10 mL) and EtOAc (10 mL). The aqueous layer was extracted with EtOAc (3×10 mL). The combined organic layers were dried (Na₂SO₄), filtered and concentrated. The crude material was purified by column chromatography (acetone in hexanes, 10-100%, a gradient elution) followed by reverse chromatography (C18 column, MeCN in water with 10 mM ammonium formate, 5-100%, a gradient elution) to provide the title compound (37.5 mg, 39%) as a solid. ¹H NMR (400 MHz, DMSO-d₆) δ 8.87 (s, 1H), 8.41 (s, 1H), 8.29 (dd, J=7.6, 1.7 Hz, 1H), 8.26 (dd, J=4.9, 1.7 Hz, 1H), 7.57 (dd, J=8.6, 5.6 Hz, 2H), 7.21 (m, J=7.6, 6.9 Hz, 3H), 6.83 (s, 1H), 4.87 (s, 1H), 4.21 (s, 2H), 3.97 (s, 3H), 1.19 (s, 6H). m/z (ESI, +ve ion)=460.4 [M+H]⁺.

### Example 272. 1-{4-[6-(1,5-dimethyl-1H-imidazol-4-yl)furo[2,3-d]pyrimidin-4-yl]-3-(4-fluorophenyl)-1H-pyrazol-1-yl}-2-methylpropan-2-ol

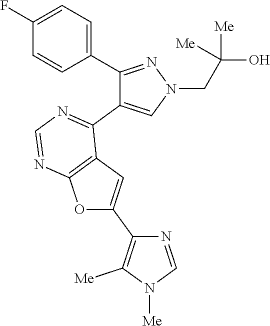 [SMILES: Cc1c(-c2cc3c(-c4cn(CC(C)(C)O)nc4-c4ccc(F)cc4)ncnc3o2)ncn1C]

### Step A. 1,5-dimethyl-4-(tributylstannyl)imidazole

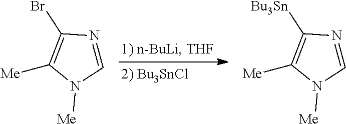

To a stirred solution of 4-bromo-1,5-dimethylimidazole (74 mg, 0.423 mmol, 1 equiv) in THF (5 mL) was added 2.5 M n-BuLi in hexanes (0.34 mL, 0.846 mmol, 2 equiv) dropwise at 0° C. under nitrogen atmosphere. The resulting mixture was stirred for 1 h at room temperature under nitrogen atmosphere. To the above mixture was added Bu₃SnCl (413 mg, 1.27 mmol, 3 equiv) dropwise over 0.5 h at 0° C. The resulting mixture was stirred for additional 1 h at room temperature. The reaction was quenched by the addition of sat. NH₄Cl (aq., 30 mL) at room temperature. The resulting mixture was extracted with EtOAc (2×60 mL). The combined organic layers were washed with brine (3×20 mL) and dried over anhydrous Na₂SO₄. After filtration, the filtrate was concentrated under reduced pressure to give the title compound (170 mg, 73%) as a colorless oil. The crude product was used in next step directly without further purification.

### Step B. 1-{4-[6-(1,5-dimethyl-1H-imidazol-4-yl)furo[2,3-d]pyrimidin-4-yl]-3-(4-fluorophenyl)-1H-pyrazol-1-yl}-2-methylpropan-2-ol

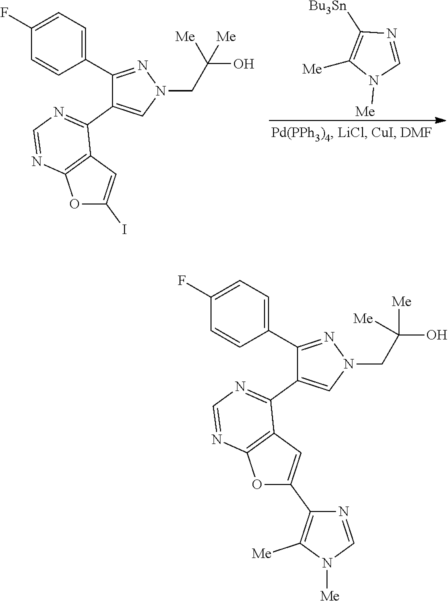

A solution of 1-[3-(4-fluorophenyl)-4-{6-iodofuro[2,3-d]pyrimidin-4-yl}pyrazol-1-yl]-2-methylpropan-2-ol (Intermediate X, 100 mg, 0.209 mmol, 1.00 equiv) in DMF (4 mL) was treated with 1,5-dimethyl-4-(tributylstannyl)imidazole (161 mg, 0.418 mmol, 2 equiv), Pd(PPh₃)₄ (48.3 mg, 0.042 mmol, 0.2 equiv), LiCl (17.73 mg, 0.418 mmol, 2 equiv) and CuI (19.9 mg, 0.104 mmol, 0.5 equiv) for 2 hours at 80° C. under nitrogen atmosphere with stirring. The mixture was allowed to cool to room temperature. To this mixture was added 100 mL EtOAc and the mixture was washed with brine (3×100 mL) and dried over anhydrous sodium sulfate. After filtration, the filtrate was concentrated under reduced pressure. The residue was purified by prep-TLC, eluted with DCM:MeOH=10:1 to afford crude product. The crude product was purified by prep-HPLC with the following conditions: Column: XBridge Shield RP18 OBD Column, 30×150 mm, 5 m; Mobile Phase A: water (10 mmol/L NH₄HCO₃), Mobile Phase B: ACN; Flow rate: 60 mL/min; Gradient: 30% B to 40% B in 8 min, 40% B; Wavelength: 254 nm; RT1 (min): 7.5 to afford the title compound (25 mg, 27%) as an off-white solid. m/z (ESI, +ve ion)=447.20 [M+H]⁺. ¹H NMR (400 MHz, DMSO-d₆) δ 8.72 (s, 1H), 8.45 (s, 1H), 7.72 (s, 1H), 7.61-7.56 (m, 2H), 7.19 (t, J=8.8 Hz, 2H), 6.62 (s, 1H), 4.84 (s, 1H), 4.19 (s, 2H), 3.61 (s, 3H), 2.46 (s, 3H), 1.18 (s, 6H).

### Example 273. 4-{1-[(1R)-2,2-difluorocyclopropyl]-3-(4-fluorophenyl)-1H-pyrazol-4-yl}-6-(1-methyl-1H-imidazol-4-yl)furo[2,3-d]pyrimidine (absolute stereochemistry arbitrarily assigned)

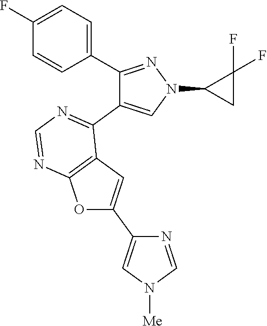 [SMILES: Cn1cnc(-c2cc3c(-c4cn([C@@H]5CC5(F)F)nc4-c4ccc(F)cc4)ncnc3o2)c1]

### Example 274. 4-{1-[(1S)-2,2-difluorocyclopropyl]-3-(4-fluorophenyl)-1H-pyrazol-4-yl}-6-(1-methyl-1H-imidazol-4-yl)furo[2,3-d]pyrimidine (absolute stereochemistry arbitrarily assigned)

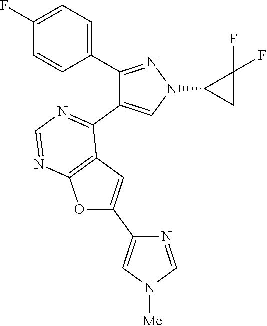 [SMILES: Cn1cnc(-c2cc3c(-c4cn([C@H]5CC5(F)F)nc4-c4ccc(F)cc4)ncnc3o2)c1]

### Step A. 1-(2,2-difluorocyclopropyl)-3-(4-fluorophenyl)-4-[6-(1-methylimidazol-4-yl)furo[2,3-d]pyrimidin-4-yl]pyrazole

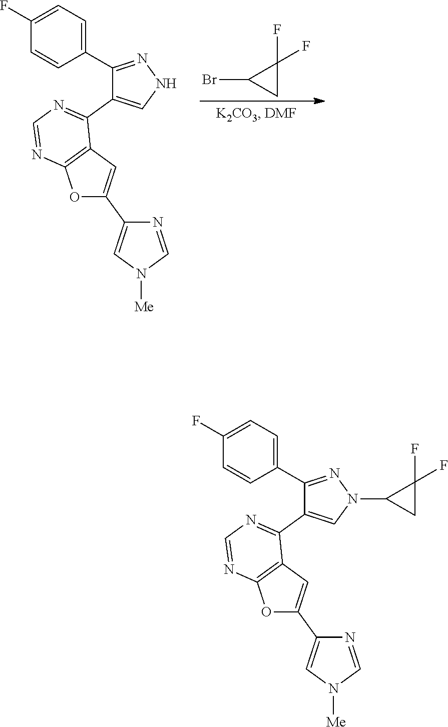 [SMILES: Cn1cnc(-c2cc3c(-c4c[nH]nc4-c4ccc(F)cc4)ncnc3o2)c1 ; FC1(F)CC1Br ; Cn1cnc(-c2cc3c(-c4cn(C5CC5(F)F)nc4-c4ccc(F)cc4)ncnc3o2)c1]

To a stirred mixture of 3-(4-fluorophenyl)-4-[6-(1-methylimidazol-4-yl)furo[2,3-d]pyrimidin-4-yl]-1H-pyrazole (Intermediate AE, 200 mg, 0.555 mmol, 1 equiv) and K₂CO₃ (153 mg, 1.11 mmol, 2 equiv) in DMF (2 mL) was added 2-bromo-1,1-difluorocyclopropane (105 mg, 0.67 mmol, 1.2 equiv) dropwise at room temperature under nitrogen atmosphere. The resulting mixture was stirred for 2 h at 60° C. under nitrogen atmosphere. The mixture was allowed to cool down to room temperature. The reaction was quenched by the addition of water (20 mL) at room temperature. The resulting mixture was extracted with EtOAc (3×40 mL). The combined organic layers were washed with brine (30 mL) and dried over anhydrous Na₂SO₄. After filtration, the filtrate was concentrated under reduced pressure. The residue was purified by silica gel column chromatography and eluted with DCM:MeOH (20:1) to afford the title compound (130 mg, 54%) as a white solid. m/z (ESI, +ve ion)=437.10 [M+H]⁺. ¹H-NMR (400 MHz, Chloroform-d) δ 8.84 (s, 1H), 8.14 (s, 1H), 7.61-7.54 (m, 3H), 7.42 (s, 1H), 7.07-7.02 (m, 2H), 6.74 (s, 1H), 4.29-4.24 (m, 1H), 3.81 (s, 3H), 2.43-2.36 (m, 1H), 2.26-2.16 (m, 1H).

### Step B. 4-{1-[(1R)-2,2-difluorocyclopropyl]-3-(4-fluorophenyl)-1H-pyrazol-4-yl}-6-(1-methyl-1H-imidazol-4-yl)furo[2,3-d]pyrimidine (absolute stereochemistry arbitrarily assigned) and 4-{1-[(1S)-2,2-difluorocyclopropyl]-3-(4-fluorophenyl)-1H-pyrazol-4-yl}-6-(1-methyl-1H-imidazol-4-yl)furo[2,3-d]pyrimidine (absolute stereochemistry arbitrarily assigned)

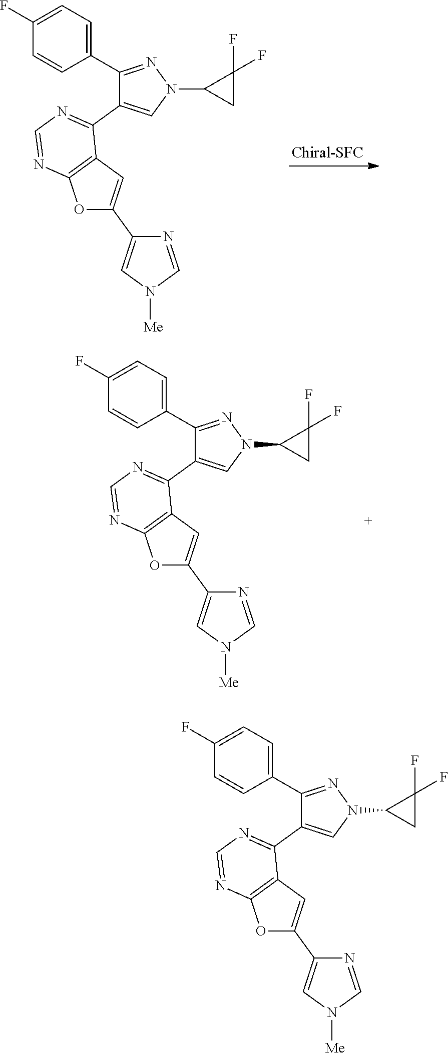

The racemic 1-(2,2-difluorocyclopropyl)-3-(4-fluorophenyl)-4-[6-(1-methylimidazol-4-yl)furo[2,3-d]pyrimidin-4-yl]pyrazole (80 mg, 0.183 mmol, 1 equiv) was separated by prep-Chiral-HPLC with the following conditions: Column: CHIRAL ART Amylose-C NEO, 3×25 cm, 5 m; Mobile Phase A: CO₂, Mobile Phase B: IPA (0.5% 2M NH₃-MeOH); Flow rate: 100 mL/min; Gradient: isocratic 35% B; Column Temperature (° C.): 35; Back Pressure (bar): 100; Wavelength: 220 nm; RT1 (min): 3.77; RT2 (min): 4.95; Sample Solvent: IPA (0.5% 2M NH₃-MeOH). The first product-containing fractions were collected and roto-evaporated in vacuo and lyophilized overnight to give Example 273 (29.7 mg, 37%) (absolute stereochemistry arbitrarily assigned) as a white solid. m/z (ESI, +ve ion)=437.15 [M+H]⁺. ¹H-NMR (400 MHz, Chloroform-d) δ 8.84 (s, 1H), 8.13 (s, 1H), 7.65-7.54 (m, 3H), 7.42 (s, 1H), 7.07-7.03 (m, 2H), 6.69 (s, 1H), 4.28-4.22 (m, 1H), 3.81 (s, 3H), 2.45-2.35 (m, 1H), 2.26-2.17 (m, 1H). The second product-containing fractions were collected and roto-evaporated in vacuo and lyophilized overnight to give Example 274 (30.8 mg, 39%) (absolute stereochemistry arbitrarily assigned) as a white solid. m/z (ESI, +ve ion)=437.20 [M+H]⁺. ¹H-NMR (400 MHz, Chloroform-d) δ 8.84 (s, 1H), 8.13 (s, 1H), 7.60-7.56 (m, 3H), 7.42 (s, 1H), 7.07-7.02 (m, 2H), 6.66 (s, 1H), 4.28-4.21 (m, 1H), 3.80 (s, 3H), 2.45-2.34 (m, 1H), 2.26-2.18 (m, 1H).

### Example 275. 1-{3-(4-fluorophenyl)-4-[6-(1-methyl-1H-pyrazol-3-yl)furo[2,3-d]pyrimidin-4-yl]-1H-pyrazol-1-yl}-2-methylpropan-2-ol

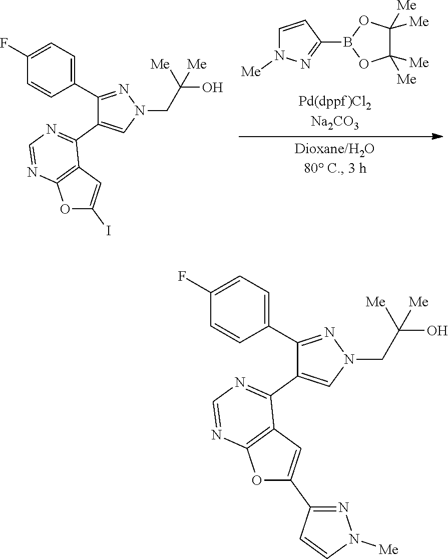 [SMILES: CC(C)(O)Cn1cc(-c2ncnc3oc(I)cc23)c(-c2ccc(F)cc2)n1 ; Cn1ccc(B2OC(C)(C)C(C)(C)O2)n1 ; Cn1ccc(-c2cc3c(-c4cn(CC(C)(C)O)nc4-c4ccc(F)cc4)ncnc3o2)n1]

Sodium carbonate (44.3 mg, 0.42 mmol, 2.00 equiv), 1-[3-(4-fluorophenyl)-4-{6-iodofuro[2,3-d]pyrimidin-4-yl}pyrazol-1-yl]-2-methylpropan-2-ol (Intermediate X, 100 mg, 0.21 mmol, 1.00 equiv) and 1-methyl-3-(4,4,5,5-tetramethyl-1,3,2-dioxaborolan-2-yl)pyrazole (43.5 mg, 0.21 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (17.1 mg, 0.02 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by reverse phase chromatography (Column: Gemini 10 μm C18 110 Å LC Column, 250×50 mm; Mobile Phase A: water (10 mmol/L NH₄HCO₃), Mobile Phase B: ACN; Flow rate: 120 mL/min; Gradient: 20% B to 65% B in 20 min; Wavelength: 254 nm; RT1 (min): 16.4) to provide the title compound (40 mg, 44%) as a white solid. m/z (ESI+ve ion)=433.25 [M+H]⁺. ¹H NMR (400 MHz, DMSO) δ 8.75 (s, 1H), 8.51 (s, 1H), 7.89 (s, 1H), 7.68-7.51 (m, 2H), 7.18 (t, J=8.7 Hz, 2H), 7.05 (s, 1H), 6.75 (s, 1H), 4.86 (s, 1H), 4.19 (s, 2H), 3.94 (s, 3H), 1.18 (s, 6H).

### Example 276. 1-{3-(4-fluorophenyl)-4-[6-(5-methoxypyridin-2-yl)furo[2,3-d]pyrimidin-4-yl]-1H-pyrazol-1-yl}-2-methylpropan-2-ol

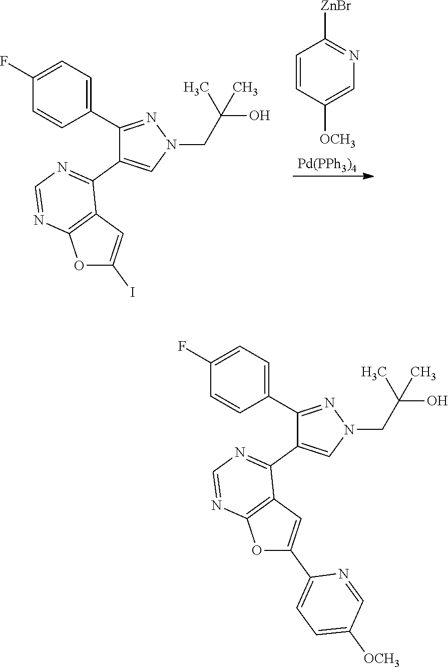

(5-Methoxypyridin-2-yl)zinc(II) bromide (0.5 M in THF, 1 mL, 500 μmol, 2.40 equiv.) was added to a degassed mixture 1-[3-(4-Fluorophenyl)-4-{6-iodofuro[2,3-d]pyrimidin-4-yl}pyrazol-1-yl]-2-methylpropan-2-ol (Intermediate X, 209 μmol, 1.00 equiv.) and tetrakis(triphenylphosphine)palladium (0) (4.83 mg, 4.18 μmol, 0.02 equiv). The mixture was stirred at room temperature for 16 h then diluted with saturated aqueous NH₄Cl (10 mL) and EtOAc (10 mL). The aqueous layer was extracted with EtOAc (3×10 mL). The combined organic layers were dried (Na₂SO₄), filtered and and concentrated under reduced pressure. The crude mixture was purified by column chromatography (EtOAc in hexanes, 10-100%, a gradient elution) followed by reverse chromatography (C18 column, MeCN in water with 10 mM ammonium formate, 5-100%, a gradient elution), to provide the title compound (49.8 mg, 51%) as a solid. ¹H NMR (400 MHz, ACN-d₃) δ 8.73 (s, 1H), 8.34 (s, 1H), 8.25 (s, 1H), 7.89 (d, J=8.8 Hz, 1H), 7.63-7.58 (m, 2H), 7.42 (dd, J=8.8, 2.9 Hz, 1H), 7.07 (t, J=8.9 Hz, 2H), 7.00 (s, 1H), 4.20 (s, 2H), 3.90 (s, 3H), 1.22 (s, 6H). m/z (ESI, +ve ion)=460.4 [M+H]⁺.

### Example 277. 1-{3-(4-fluorophenyl)-4-[6-(5-fluoropyridin-2-yl)furo[2,3-d]pyrimidin-4-yl]-1H-pyrazol-1-yl}-2-methylpropan-2-ol

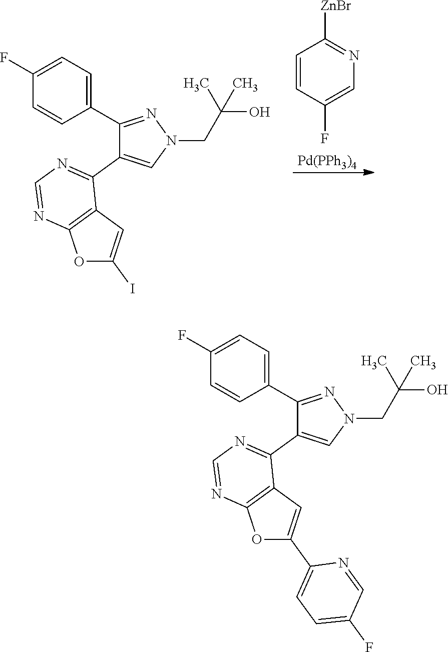

5-Fluoro-2-pyridylzinc bromide solution (0.5 M in THF, 627 μL, 314 μmol, 1.5 equiv) was added to a degassed mixture 1-[3-(4-Fluorophenyl)-4-{6-iodofuro[2,3-d]pyrimidin-4-yl}pyrazol-1-yl]-2-methylpropan-2-ol (Intermediate X, 100 mg, 209 μmol, 1.0 equiv) and tetrakis(triphenylphosphine)palladium (0) (2.47 mg, 2.09 μmol, 0.01 equiv). The mixture was stirred at room temperature for 24 h. Tetrakis(triphenylphosphine)palladium(0) (2.47 mg, 2.09 μmol, 0.01 equiv) and 5-fluoro-2-pyridylzinc bromide solution (0.5 M in THF, 627 μL, 314 μmol, 1.5 equiv) were added and the mixture was stirred at room temperature for 24 h and then diluted with saturated aqueous NH₄Cl (2 mL). The aqueous layer was extracted with EtOAc (2×5 mL). The combined organic layers were washed with brine, then dried (MgSO₄), filtered and concentrated under reduced pressure. The crude material was purified by column chromatography (EtOAc in hexanes, 15-100%, a gradient elution), to provide the title compound (54.7 mg, 58%) as a solid. ¹H NMR (400 MHz, DMSO-d₆) δ 8.82 (s, 1H), 8.75 (d, J=2.9 Hz, 1H), 8.57 (s, 1H), 8.08 (dd, J=8.8, 4.4 Hz, 1H), 7.95 (td, J=8.7, 2.9 Hz, 1H), 7.64-7.57 (m, 2H), 7.45 (s, 1H), 7.23-7.14 (m, 2H), 4.87 (s, 1H), 4.20 (s, 2H), 1.19 (s, 6H). m/z (ESI, +ve ion)=448.3 [M+H]⁺.

### Example 278. (1r,3r)-3-{3-(4-fluorophenyl)-4-[6-(1-methyl-1H-imidazol-4-yl)furo[2,3-d]pyrimidin-4-yl]-1H-pyrazol-1-yl}cyclobutan-1-ol

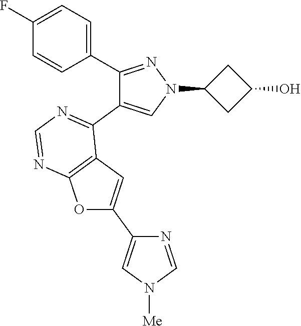 [SMILES: Cn1cnc(-c2cc3c(-c4cn([C@H]5C[C@H](O)C5)nc4-c4ccc(F)cc4)ncnc3o2)c1]

### Step A. 3-(4-fluorophenyl)-4-[6-(1-methylimidazol-4-yl)furo[2,3-d]pyrimidin-4-yl]-1-[3-(benzyloxy)cyclobutyl]pyrazole

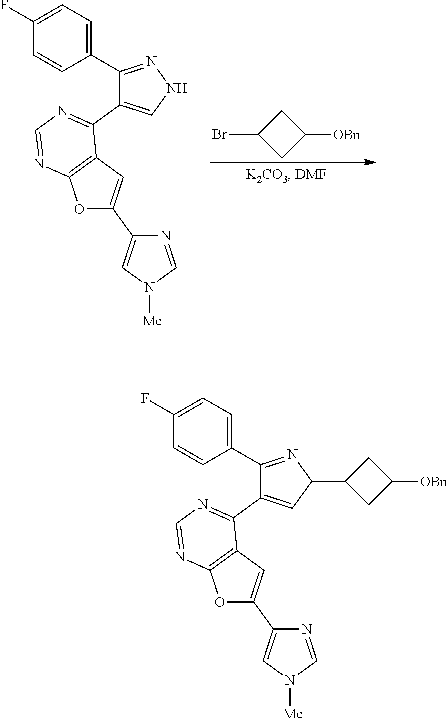 [SMILES: Cn1cnc(-c2cc3c(-c4c[nH]nc4-c4ccc(F)cc4)ncnc3o2)c1 ; BrC1CC(OCc2ccccc2)C1 ; Cn1cnc(-c2cc3c(C4=CC(C5CC(OCc6ccccc6)C5)N=C4c4ccc(F)cc4)ncnc3o2)c1]

To a stirred mixture of 3-(4-fluorophenyl)-4-[6-(1-methylimidazol-4-yl)furo[2,3-d]pyrimidin-4-yl]-1H-pyrazole (Intermediate AE, 350 mg, 0.971 mmol, 1 equiv) and K₂CO₃ (269 mg, 1.94 mmol, 2 equiv) in DMF (3 mL) was added [(3-bromocyclobutoxy)methyl]benzene (281 mg, 1.17 mmol, 1.2 equiv) at room temperature under nitrogen atmosphere. The resulting mixture was stirred for 2 h at 100° C. under nitrogen atmosphere. The reaction was quenched with water (10 mL) at room temperature. The resulting mixture was extracted with EtOAc (3×10 mL). The combined organic layers were washed with brine (15 mL) and dried over anhydrous Na₂SO₄. After filtration, the filtrate was concentrated under reduced pressure. The residue was purified by silica gel column chromatography eluting with DCM:MeOH (10:1) to afford the crude compound as a yellow solid and then further purified by prep-TLC (DCM:MeOH 10:1) to afford the title compound (218 mg, 43%) as an off-white solid. m/z (ESI, +ve ion)=521.20 [M+H]⁺.

### Step B. 3-(4-fluorophenyl)-4-[6-(1-methylimidazol-4-yl)furo[2,3-d]pyrimidin-4-yl]-1-[(trans)-3-(benzyloxy)cyclobutyl]pyrazole

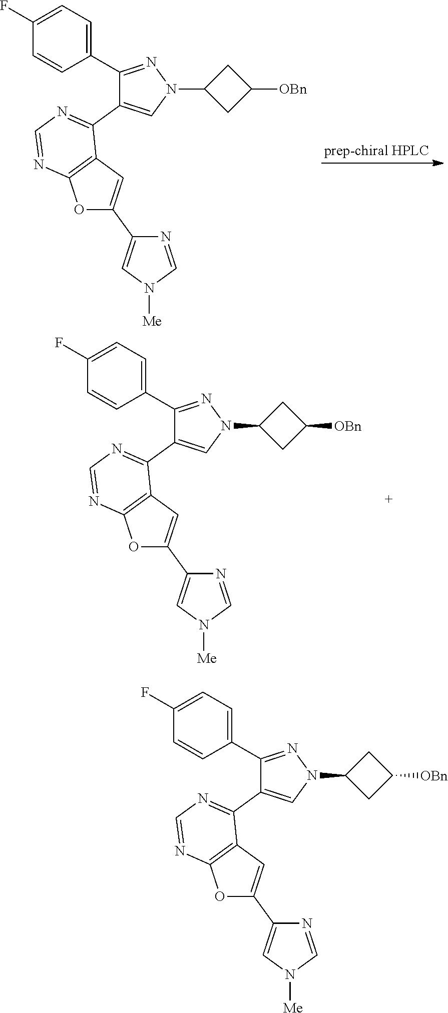

The 1-[3-(benzyloxy)cyclobutyl]-3-(4-fluorophenyl)-4-[6-(1-methylimidazol-4-yl)furo[2,3-d]pyrimidin-4-yl]pyrazole (218 mg, 0.419 mmol, 1 equiv) was separated by prep-Chiral-HPLC with the following conditions: Column: CHIRALPAK IG, 2×25 cm, 5 μm; Mobile Phase A: Hex (0.5% 2M NH₃-MeOH)-HPLC, Mobile Phase B: MeOH:DCM=1:1-HPLC; Flow rate: 20 mL/min; Gradient: 40% B to 40% B in 11 min; Wavelength: 220/254 nm; RT1 (min): 7.57; RT2 (min): 9.97; Sample Solvent: MeOH:DCM=1:1-HPLC. The first product-containing fractions were collected and roto-evaporated in vacuo and lyophilized overnight to give 3-(4-fluorophenyl)-4-[6-(1-methylimidazol-4-yl)furo[2,3-d]pyrimidin-4-yl]-1-[(cis)-3-(benzyloxy)cyclobutyl]pyrazole (70 mg, 32%) as a white solid. The second product-containing fractions were collected and roto-evaporated in vacuo and lyophilized overnight to give the title compound (138 mg, 63%) as a white solid. m/z (ESI, +ve ion)=521.20 [M+H]⁺.

### Step C. (1r,3r)-3-{3-(4-fluorophenyl)-4-[6-(1-methyl-1H-imidazol-4-yl)furo[2,3-d]pyrimidin-4-yl]-1H-pyrazol-1-yl}cyclobutan-1-ol

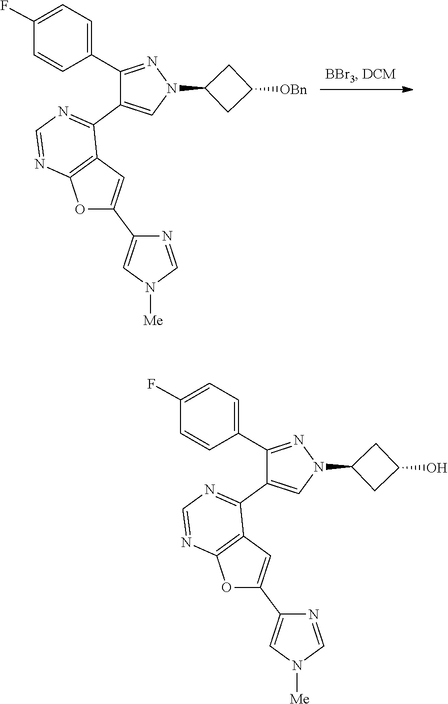 [SMILES: Cn1cnc(-c2cc3c(-c4cn([C@H]5C[C@H](OCc6ccccc6)C5)nc4-c4ccc(F)cc4)ncnc3o2)c1 ; Cn1cnc(-c2cc3c(-c4cn([C@H]5C[C@H](O)C5)nc4-c4ccc(F)cc4)ncnc3o2)c1]

To a stirred mixture of 3-(4-fluorophenyl)-4-[6-(1-methylimidazol-4-yl)furo[2,3-d]pyrimidin-4-yl]-1-[(trans)-3-(benzyloxy)cyclobutyl]pyrazole (70 mg, 0.134 mmol, 1 equiv) in DCM (1 mL) was added BBr₃ (124.64 mg, 0.496 mmol, 3.7 equiv) dropwise at 0° C. under nitrogen atmosphere. The resulting mixture was stirred for 3 h at room temperature under nitrogen atmosphere. The reaction was quenched by the addition of sat. NaHCO₃ (aq., 10 mL) at room temperature. The resulting mixture was extracted with EtOAc (3×10 mL). The combined organic layers were washed with brine (15 mL) and dried over anhydrous Na₂SO₄. After filtration, the filtrate was concentrated under reduced pressure. The residue was purified by reverse flash chromatography with the following conditions: column, C18 silica gel; mobile phase, MeCN in Water (10 mmol/L NH₄HCO₃), 20% to 60% gradient in 20 min; detector, UV 254 nm, to provide the title compound (25.8 mg, 43%) as a white solid. m/z (ESI, +ve ion)=431.20 [M+H]⁺. ¹H NMR (400 MHz, DMSO-d₆) δ 8.70 (s, 1H), 8.67 (s, 1H), 7.82-7.72 (m, 2H), 7.64-7.56 (m, 2H), 7.24-7.14 (m, 2H), 6.87 (s, 1H), 5.26 (d, J=4.8 Hz, 1H), 5.20-5.11 (m, 1H), 4.54 (d, J=4.4 Hz, 1H), 3.74 (s, 3H), 2.86-2.75 (m, 2H), 2.52-2.41 (m, 2H).

### Example 279. (1r,3r)-3-{4-[6-(1,3-dimethyl-1H-pyrazol-4-yl)furo[2,3-d]pyrimidin-4-yl]-3-(4-fluorophenyl)-1H-pyrazol-1-yl}cyclobutane-1-carbonitrile

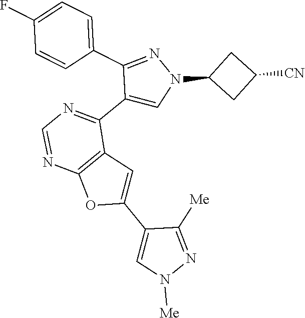 [SMILES: Cc1nn(C)cc1-c1cc2c(-c3cn([C@H]4C[C@H](C#N)C4)nc3-c3ccc(F)cc3)ncnc2o1]

### Step A. 4-[6-(1,3-dimethylpyrazol-4-yl)furo[2,3-d]pyrimidin-4-yl]-3-(4-fluorophenyl)-1H-pyrazole

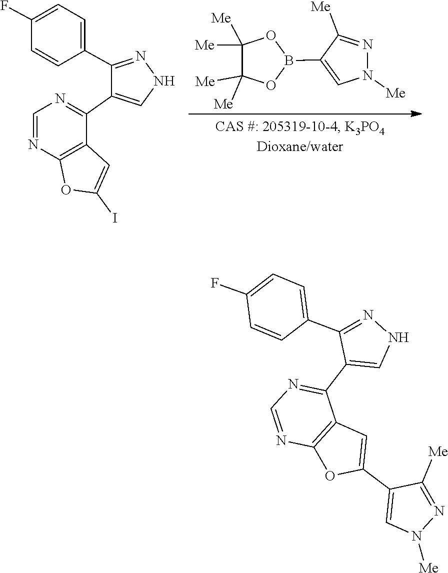 [SMILES: Fc1ccc(-c2n[nH]cc2-c2ncnc3oc(I)cc23)cc1 ; Cc1nn(C)cc1B1OC(C)(C)C(C)(C)O1 ; Cc1nn(C)cc1-c1cc2c(-c3c[nH]nc3-c3ccc(F)cc3)ncnc2o1]

A solution of 3-(4-fluorophenyl)-4-{6-iodofuro[2,3-d]pyrimidin-4-yl}-1H-pyrazole (Intermediate T, 1.1 g, 2.708 mmol, 1 equiv) in 1,4-dioxane (28 mL) and water (7 mL) was treated with dichloro[9,9-dimethyl-4,5-bis(diphenylphosphino)xanthene]palladium(II) (21 g, 0.271 mmol, 0.1 equiv) and K₃PO₄ (1.15 g, 5.416 mmol, 2 equiv) for 16 hour at 80° C. under nitrogen atmosphere with stirring. The reaction was quenched by the addition of saturated ammonium chloride aqueous solution (100 mL). The resulting mixture was extracted with ethyl acetate (3×200 mL). The combined organic layers were washed with brine (3×200 mL) and dried over anhydrous sodium sulfate. After filtration, the filtrate was concentrated under reduced pressure. The residue was purified by silica gel column chromatography and eluted with 20%-40% methanol in dichloromethane to provide the title compound (600 mg, 49%) as a yellow solid. m/z (ESI, +ve ion)=374.95 [M+H]⁺.

### Step B. (1r,3r)-3-{4-[6-(1,3-dimethyl-1H-pyrazol-4-yl)furo[2,3-d]pyrimidin-4-yl]-3-(4-fluorophenyl)-1H-pyrazol-1-yl}cyclobutane-1-carbonitrile

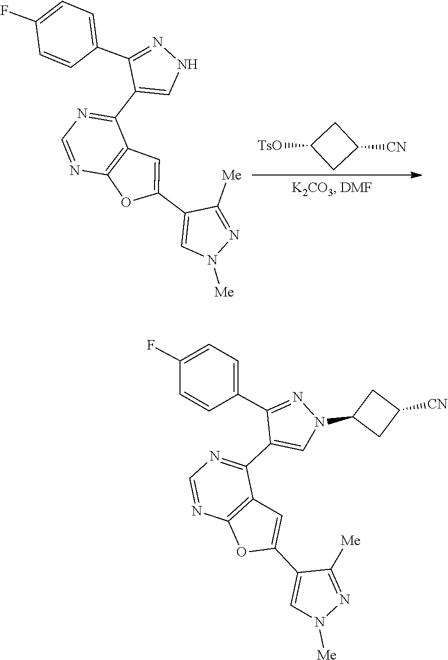 [SMILES: Cc1nn(C)cc1-c1cc2c(-c3c[nH]nc3-c3ccc(F)cc3)ncnc2o1 ; Cc1ccc(S(=O)(=O)O[C@H]2C[C@@H](C#N)C2)cc1 ; Cc1nn(C)cc1-c1cc2c(-c3cn([C@H]4C[C@H](C#N)C4)nc3-c3ccc(F)cc3)ncnc2o1]

A solution of 4-[6-(1,3-dimethylpyrazol-4-yl)furo[2,3-d]pyrimidin-4-yl]-3-(4-fluorophenyl)-1H-pyrazole (120 mg, 0.321 mmol, 1 equiv) in DMF (6 mL) was treated with (cis)-3-cyanocyclobutyl 4-methylbenzenesulfonate (Intermediate AD, 121 mg, 0.482 mmol, 1.5 equiv) and K₂CO₃ (132.90 mg, 0.963 mmol, 3 equiv) for 16 hours at 80° C. under nitrogen atmosphere with stirring. The mixture was allowed to cool to room temperature. The reaction solution was purified by prep-HPLC with the following conditions: Column: XBridge Shield RP18 OBD Column, 30×150 mm, 5 m; Mobile Phase A: Water (10 mmol/L NH₄HCO₃), Mobile Phase B: ACN; Flow rate: 60 mL/min; Gradient: 35% B to 45% B in 10 min, 45% B; Wavelength: 254 nm; RT1 (min): 8; which provided the title compound (34.0 mg, 23%) as an off-white solid. m/z (ESI, +ve ion)=454.15 [M+H]⁺. ¹H NMR (400 MHz, DMSO-d₆) δ 8.86 (s, 1H), 8.05 (s, 1H), 7.71 (s, 1H), 7.53 (t, J=6.8 Hz, 2H), 7.08 (t, J=8.4 Hz, 2H), 5.66 (s, 1H), 5.25-5.13 (m, 1H), 3.88 (s, 3H), 3.47-3.35 (m, 1H), 3.30-3.15 (m, 2H), 3.09-2.89 (m, 2H), 2.13 (s, 3H).

### Example 280. 1-[4-{6-[5-(difluoromethyl)-1,3-dimethyl-1H-pyrazol-4-yl]furo[2,3-d]pyrimidin-4-yl}-3-(4-fluorophenyl)-1H-pyrazol-1-yl]-2-methylpropan-2-ol

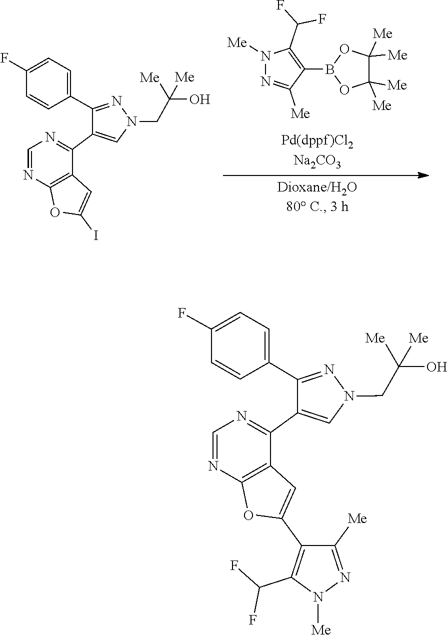 [SMILES: CC(C)(O)Cn1cc(-c2ncnc3oc(I)cc23)c(-c2ccc(F)cc2)n1 ; Cc1nn(C)c(C(F)F)c1B1OC(C)(C)C(C)(C)O1 ; Cc1nn(C)c(C(F)F)c1-c1cc2c(-c3cn(CC(C)(C)O)nc3-c3ccc(F)cc3)ncnc2o1]

Sodium carbonate (44.3 mg, 0.42 mmol, 2.00 equiv), 1-[3-(4-fluorophenyl)-4-{6-iodofuro[2,3-d]pyrimidin-4-yl}pyrazol-1-yl]-2-methylpropan-2-ol (Intermediate X, 100 mg, 0.21 mmol, 1.00 equiv) and 5-(difluoromethyl)-1,3-dimethyl-4-(4,4,5,5-tetramethyl-1,3,2-dioxaborolan-2-yl)pyrazole (56.9 mg, 0.21 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (17.1 mg, 0.02 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by reverse phase chromatography (Column: Gemini 10 μm C18 110 Å LC Column, 250×50 mm; Mobile Phase A: water (10 mmol/L NH₄HCO₃), Mobile Phase B: ACN; Flow rate: 120 mL/min; Gradient: 20% B to 75% B in 22 min; Wavelength: 254 nm; RT1 (min): 21) to provide the title compound (46 mg, 44%) as a white solid. m/z (ESI+ve ion)=497.15 [M+H]⁺. ¹H NMR (400 MHz, DMSO) δ 8.83 (s, 1H), 8.41 (s, 1H), 7.55 (dd, J=8.7, 5.7 Hz, 2H), 7.37 (t, J=51.8 Hz, 1H), 7.19 (t, J=8.9 Hz, 2H), 6.48 (s, 1H), 4.83 (s, 1H), 4.18 (s, 2H), 3.96 (s, 3H), 2.20 (s, 3H), 1.18 (s, 6H).

### Example 281. 1-{3-(4-fluorophenyl)-4-[6-(6-fluoropyridin-2-yl)furo[2,3-d]pyrimidin-4-yl]-1H-pyrazol-1-yl}-2-methylpropan-2-ol

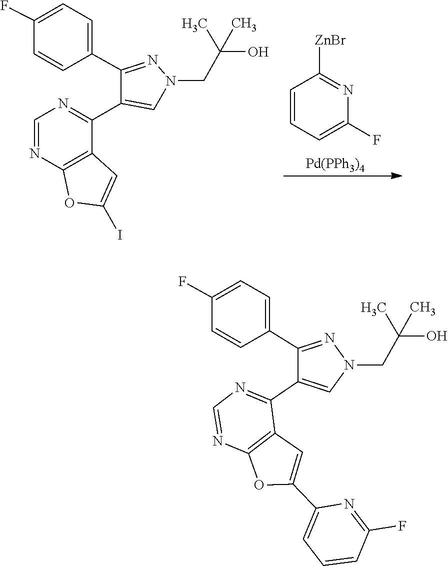

6-Fluoro-2-pyridylzinc bromide solution (0.5 M in THF, 2.92 mL, 1.46 mmol, 7.0 equiv) was added to a degassed mixture of 1-[3-(4-Fluorophenyl)-4-{6-iodofuro[2,3-d]pyrimidin-4-yl}pyrazol-1-yl]-2-methylpropan-2-ol (Intermediate X, 100 mg, 209 μmol, 1.0 equiv) and tetrakis(triphenylphosphine)palladium (0) (24.7 mg, 20.9 μmol, 0.1 equiv). The mixture was stirred for 2 hours and then diluted with water (10 mL). The aqueous layer was extracted with EtOAc (2×25 mL). The combined organic layers were washed with brine then dried (MgSO₄), filtered and concentrated under reduced pressure. The crude product was purified by column chromatography (EtOAc in hexane, 0-100%, a gradient elution) to provide the title compound (28.7 mg, 29%) as a solid. ¹H NMR (400 MHz, DMSO-d₆) δ 8.84 (s, 1H), 8.60 (s, 1H), 8.20 (dd, J=15.9, 8.2 Hz, 1H), 7.94 (dd, J=7.5, 2.3 Hz, 1H), 7.65-7.57 (m, 2H), 7.50 (s, 1H), 7.30 (dd, J=8.2, 2.1 Hz, 1H), 7.24-7.12 (m, 2H), 4.87 (s, 1H), 4.21 (s, 2H), 1.19 (s, 6H). m/z (ESI, +ve ion)=448.2 [M+H]⁺.

### Example 282. 1-{4-[6-(5-fluoro-6-methylpyridin-2-yl)furo[2,3-d]pyrimidin-4-yl]-3-(4-fluorophenyl)-1H-pyrazol-1-yl}-2-methylpropan-2-ol

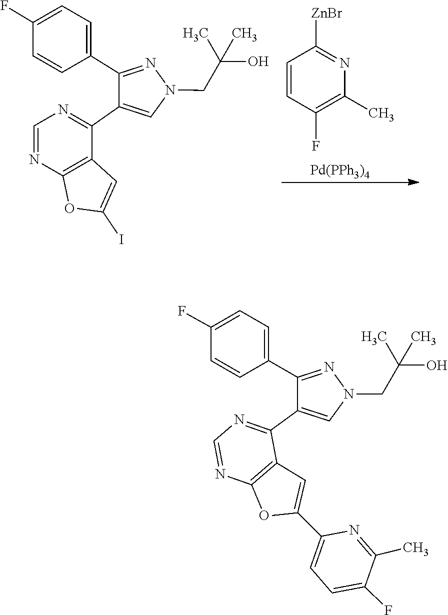

5-Fluoro-6-methyl-2-pyridylzinc bromide solution (0.5 M in THF, 418 μL, 209 μmol, 2.00 equiv.) was added to a degassed mixture of 1-[3-(4-fluorophenyl)-4-{6-iodofuro[2,3-d]pyrimidin-4-yl}pyrazol-1-yl]-2-methylpropan-2-ol (Intermediate X, 50.0 mg, 105 μmol, 1.00 equiv.) and tetrakis(triphenylphosphine)palladium (0) (1.23 mg, 1.05 μmol, 0.01 equiv.). The mixture was stirred at room temperature for 2 days. The mixture was diluted with saturated aqueous NH₄Cl. The aqueous layer was extracted with EtOAc (3×10 mL). The combined organic layers were washed with saturated aqueous NaHCO₃, brine then dried (Na₂SO₄), filtered and concentrated under reduced pressure. The crude material was purified by reverse phase chromatography (C18 column, MeCN in water with 10 mM ammonium formate, 10-100%, a gradient elution) followed by column chromatography (EtOAc in heptanes, 0-100%, a gradient elution) to provide the title compound (26.5 mg, 55%) as a solid. ¹H NMR (400 MHz, DMSO-d₆) δ 8.82 (s, 1H), 8.56 (s, 1H), 7.90 (dd, J=8.6, 3.8 Hz, 1H), 7.83 (t, J=8.9 Hz, 1H), 7.63-7.57 (m, 2H), 7.29 (s, 1H), 7.23-7.16 (m, 2H), 4.86 (s, 1H), 4.21 (s, 2H), 2.54 (d, J=2.8 Hz, 3H), 1.19 (s, 6H). MeOH. m/z (ESI, +ve ion)=462.2 [M+H]⁺.

### Example 283. 3-{4-[3-(4-fluorophenyl)-1-(2-hydroxy-2-methylpropyl)-1H-pyrazol-4-yl]furo[2,3-d]pyrimidin-6-yl}pyridin-2-ol

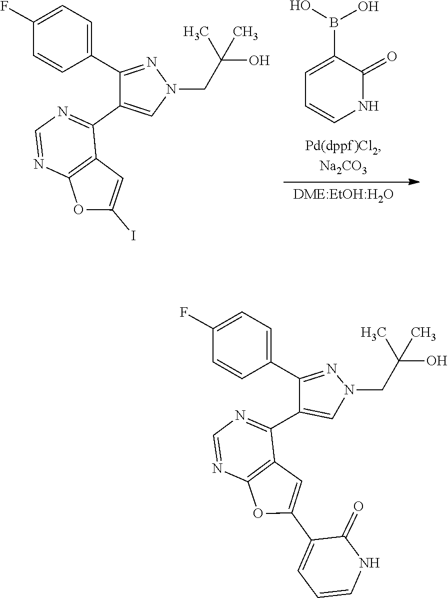

1,1′-Bis(diphenylphosphino)ferrocene dichloropalladium (II) (16.1 mg, 20.9 μmol, 0.1 equiv.) was added to a degassed mixture of 1-[3-(4-fluorophenyl)-4-{6-iodofuro[2,3-d]pyrimidin-4-yl}pyrazol-1-yl]-2-methylpropan-2-ol (Intermediate X, 100 mg, 209 μmol, 1.0 equiv.), (2-oxo-1,2-dihydropyridin-3-yl)boronic acid (32.0 mg, 230 μmol, 1.1 equiv.) and sodium carbonate (67.2 mg, 627 μmol, 3.0 equiv.) in a mixture of DME (1.36 mL), EtOH (678 μL) and H₂O (339 μL). The mixture was degassed and stirred at 85° C. for 16 hours. The mixture was cooled to room temperature and diluted with water. The aqueous layer was extracted with EtOAc (3×20 mL). The combined organic layers were washed with brine then dried (Na₂SO₄), filtered and concentrated under reduced pressure. The crude material was purified by reverse phase chromatography (C18 column, MeCN in water with 10 mM ammonium formate, 10-50%, a gradient elution) to provide the title compound (21.0 mg, 23%) as a solid. ¹H NMR (400 MHz, DMSO-d₆) δ 12.24 (s, 1H), 8.78 (s, 1H), 8.37 (s, 1H), 8.14 (dd, J=7.2, 2.0 Hz, 1H), 7.60-7.51 (m, 3H), 7.42 (s), 7.20-7.11 (m, 1H), 6.47-6.41 (m, 2H), 4.86 (s, 1H), 4.20 (s, 2H), 1.17 (s, 6H). m/z (ESI, +ve ion)=446.2 [M+H]⁺.

### Example 284. 1-(4-(6-(7,7-dimethyl-6,7-dihydro-5H-pyrazolo[5,1-b][,3]oxazin-3-yl)furo[2,3-d]pyrimidin-4-yl)-3-(4-fluorophenyl)-1H-pyrazol-1-yl)-2-methylpropan-2-ol

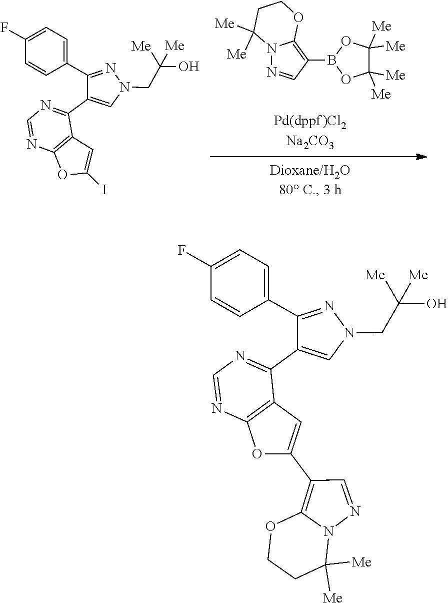 [SMILES: CC(C)(O)Cn1cc(-c2ncnc3oc(I)cc23)c(-c2ccc(F)cc2)n1 ; CC1(C)CCOc2c(B3OC(C)(C)C(C)(C)O3)cnn21 ; CC(C)(O)Cn1cc(-c2ncnc3oc(-c4cnn5c4OCCC5(C)C)cc23)c(-c2ccc(F)cc2)n1]

Sodium carbonate (44.3 mg, 0.42 mmol, 2.00 equiv), 1-[3-(4-fluorophenyl)-4-{6-iodofuro[2,3-d]pyrimidin-4-yl}pyrazol-1-yl]-2-methylpropan-2-ol (Intermediate X, 100 mg, 0.21 mmol, 1.00 equiv) and 7,7-dimethyl-3-(4,4,5,5-tetramethyl-1,3,2-dioxaborolan-2-yl)-5,6-dihydropyrazolo[5,1-b][1,3]oxazine (58.2 mg, 0.21 mmol, 1.00 equiv) were suspended in a mixture of 1,4-dioxane (5 mL) and water (1 mL) under nitrogen atmosphere. The mixture was degassed with nitrogen for 5 min and [1,1′-bis(diphenylphosphino)ferrocene]palladium(II) dichloride dichloromethane adduct (17.1 mg, 0.02 mmol, 0.10 equiv) was added. The mixture was further degassed for 5 min before the reaction mixture was heated at 80° C. for 3 h under positive nitrogen pressure. After cooling to rt, the mixture was filtered through a pad of celite and washed with EtOAc. The solvent was removed under reduced pressure to give the crude material as an oil. The residue was purified by reverse phase chromatography (Column: Gemini 10 μm C18 110 Å LC Column, 250×50 mm; Mobile Phase A: water (10 mmol/L NH₄HCO₃), Mobile Phase B: ACN; Flow rate: 120 mL/min; Gradient: 20% B to 65% B in 20 min; Wavelength: 254 nm; RT1 (min): 19.5) to provide the title compound (30 mg, 29%) as a white solid. m/z (ESI+ve ion)=503.15 [M+H]⁺. ¹H NMR (400 MHz, DMSO-d₆) δ 8.70 (s, 1H), 8.35 (s, 1H), 7.75 (s, 1H), 7.58-7.49 (m, 2H), 7.24-7.15 (m, 2H), 6.11 (s, 1H), 4.83 (s, 1H), 4.54-4.44 (m, 2H), 4.18 (s, 2H), 2.20 (t, J=5.4 Hz, 2H), 1.52 (s, 6H), 1.17 (s, 6H).

### Example 285. 3-{3-(4-fluorophenyl)-4-[6-(2-methoxypyridin-3-yl)furo[2,3-d]pyrimidin-4-yl]-1H-pyrazol-1-yl}propanenitrile

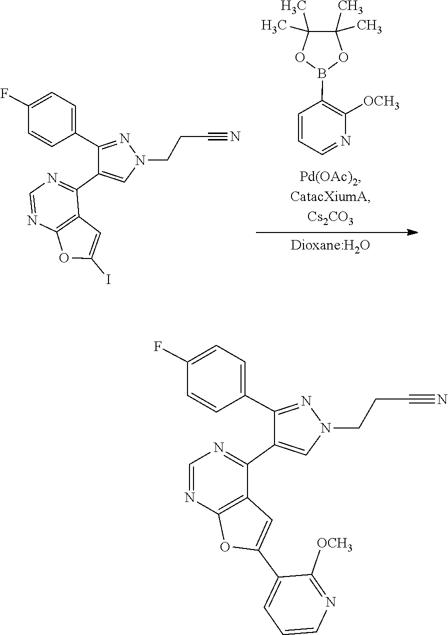 [SMILES: N#CCCn1cc(-c2ncnc3oc(I)cc23)c(-c2ccc(F)cc2)n1 ; COc1ncccc1B1OC(C)(C)C(C)(C)O1 ; COc1ncccc1-c1cc2c(-c3cn(CCC#N)nc3-c3ccc(F)cc3)ncnc2o1]

Palladium(II) acetate (2.44 mg, 10.9 μmol, 0.05 equiv.), butyldi-1-adamantylphosphine (8.22 mg, 21.8 μmol, 0.1 equiv.) were added to a degassed mixture of 3-(3-(4-fluorophenyl)-4-(6-iodofuro[2,3-d]pyrimidin-4-yl)-1H-pyrazol-1-yl)propanenitrile (Intermediate AB, 100 mg, 218 μmol, 1.0 equiv.), 2-methoxy-3-(4,4,5,5-tetramethyl-[1,3,2]dioxaborolan-2-yl)-pyridine (64.7 mg, 261 μmol, 1.20 equiv.) and cesium carbonate (145 mg, 436 μmol, 2.0 equiv.) in a mixture of dioxane (500 μL) and H₂O (50 μL). The mixture was stirred at 85° C. for 2 h and then cooled to room temperature and filtered through Celite® and rinsed with EtOAc. The filtrate was concentrated under reduced pressure and diluted with saturated aqueous NH₄Cl (10 mL) and EtOAc (10 mL). The aqueous layer was extracted with EtOAc (3×10 mL). The combined organic layers were washed with brine then dried (Na₂SO₄), filtered and concentrated under reduced pressure. The crude material was purified by column chromatography (EtOAc in heptanes, 0-100%, a gradient elution) then by column chromatography (THF in heptanes, 10-100% a gradient elution), followed by reverse chromatography (C18 column, MeCN in water with 10 mM ammonium formate, 5-100%, a gradient elution) to provide the title compound (12.8 mg, 13%) as a solid. ¹H NMR (400 MHz, DMSO-d₆) δ 8.88 (s, 1H), 8.63 (s, 1H), 8.30 (dd, J=7.6, 1.8 Hz), 8.27 (dd, J=4.9, 1.8 Hz), 7.58 (dd, J=8.6, 5.6 Hz, 2H), 7.27-7.17 (m, 3H), 6.89 (s, 1H), 4.59 (t, J=6.3 Hz, 2H), 3.97 (s, 3H), 3.23 (s, 2H). m/z (ESI, +ve ion)=441.4 [M+H]⁺.

### Example 286. 2,2-difluoro-3-{3-(4-fluorophenyl)-4-[6-(2-methoxypyridin-3-yl)furo[2,3-d]pyrimidin-4-yl]-1H-pyrazol-1-yl}propan-1-ol

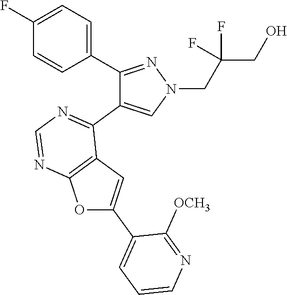 [SMILES: COc1ncccc1-c1cc2c(-c3cn(CC(F)(F)CO)nc3-c3ccc(F)cc3)ncnc2o1]

### Step A. 2,2-Difluoro-3-(3-(4-fluorophenyl)-4-(6-iodofuro[2,3-d]pyrimidin-4-yl)-1H-pyrazol-1-yl)propan-1-ol

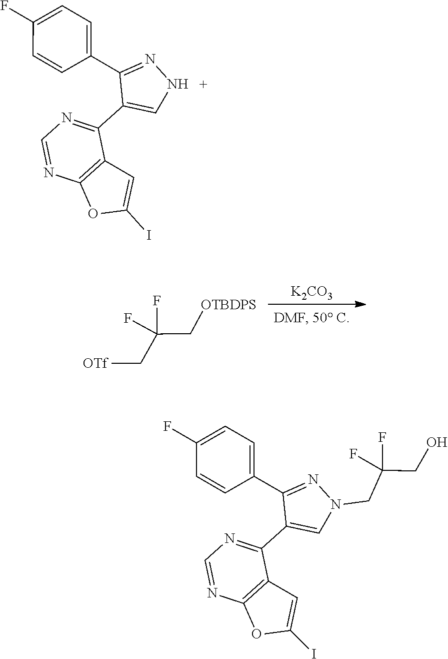

3-((tert-Butyldiphenylsilyl)oxy)-2,2-difluoropropyl trifluoromethanesulfonate (1.16 mL, 2.95 mmol, 1.2 equiv.) was added to a mixture of 3-(4-fluorophenyl)-4-{6-iodofuro[2,3-d]pyrimidin-4-yl}-1H-pyrazole (Intermediate T, 1.00 g, 2.46 mmol, 1.0 equiv.) and potassium carbonate (694 mg, 4.92 mmol, 2.0 equiv.) in DMF (24.6 mL). The mixture was stirred for 5 h at 50° C. then 2 days at room temperature. The mixture was cooled to room temperature, diluted with water (200 mL), and extracted with EtOAc (3×100 mL). The combined organic layers were washed with water, brine, then dried (Na₂SO₄), filtered and concentrated. The crude was purified by column chromatography (EtOAc in heptanes, 0-100%, a gradient elution), to provide the title compound (830 mg, 67%) as a solid. ¹H NMR (400 MHz, DMSO-d₆) δ 8.68 (s, 1H), 8.57 (s, 1H), 7.55-7.49 (m, 2H), 7.18-7.12 (m, 2H), 7.10 (s, 1H), 5.75 (t, J=6.1 Hz, 1H), 4.78 (t, J=14.1 Hz, 2H), 3.69 (tt, J=16.5, 8.1 Hz, 2H). Contains 10% of N² regioisomer. m/z (ESI, +ve ion)=501.0 [M+H]⁺.

### Step B. 2,2-difluoro-3-{3-(4-fluorophenyl)-4-[6-(2-methoxypyridin-3-yl)furo[2,3-d]pyrimidin-4-yl]-1H-pyrazol-1-yl}propan-1-ol

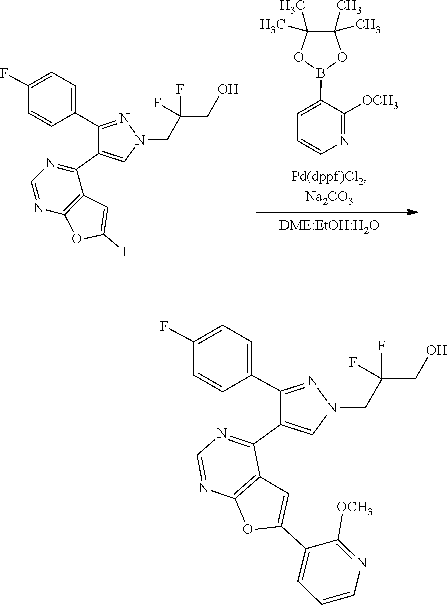 [SMILES: OCC(F)(F)Cn1cc(-c2ncnc3oc(I)cc23)c(-c2ccc(F)cc2)n1 ; COc1ncccc1B1OC(C)(C)C(C)(C)O1 ; COc1ncccc1-c1cc2c(-c3cn(CC(F)(F)CO)nc3-c3ccc(F)cc3)ncnc2o1]

1,1′-Bis(diphenylphosphino)ferrocene dichloropalladium (II) (23.1 mg, 30.0 μmol, 0.1 equiv.) was added to a degassed mixture of 2,2-difluoro-3-(3-(4-fluorophenyl)-4-(6-iodofuro[2,3-d]pyrimidin-4-yl)-1H-pyrazol-1-yl)propan-1-ol (150 mg, 300 μmol, 1.0 equiv.), 2-methoxy-3-(4,4,5,5-tetramethyl-[1,3,2]dioxaborolan-2-yl)-pyridine (81.6 mg, 330 μmol, 1.1 equiv.) and sodium carbonate (96.3 mg, 900 μmol, 3.0 equiv.) in a mixture of DME (1.94 mL), EtOH (972 μL) and H₂O (486 μL). The mixture was stirred at 85° C. for 16 hours. The mixture was cooled to room temperature and diluted with water. The aqueous layer was extracted with EtOAc (3×20 mL). The combined organic layers were washed with brine, then dried (Na₂SO₄), filtered and concentrated under reduced pressure. The crude material was purified by reverse phase chromatography (C18 column, MeCN in water with 10 mM ammonium formate, 10-100%, a gradient elution) followed by column chromatography (MeCN in DCM, 0-50%, a gradient elution) followed by Prep-HPLC (MeCN in AmF, 45-100%, a gradient elution) to provide the title compound (27.5 mg, 19%) as a solid. ¹H NMR (400 MHz, DMSO-d₆) δ 8.90 (s, 1H), 8.54 (s, 1H), 8.29 (dd, J=7.6, 1.8 Hz, 1H), 8.26 (dd, J=4.9, 1.8 Hz, 1H), 7.62-7.52 (m, 2H), 7.28-7.17 (m, 3H), 6.79 (s, 1H), 5.82 (t, J=5.9 Hz, 1H), 4.89 (t, J=14.2 Hz, 2H), 3.96 (s, 3H), 3.76 (td, J=13.5, 5.0 Hz, 2H). m/z (ESI, +ve ion)=482.1 [M+H]⁺.

### Example 287. 3-(4-{1-[(1s,3s)-3-fluorocyclobutyl]-3-(4-fluorophenyl)-1H-pyrazol-4-yl}furo[2,3-d]pyrimidin-6-yl)pyridin-2-ol

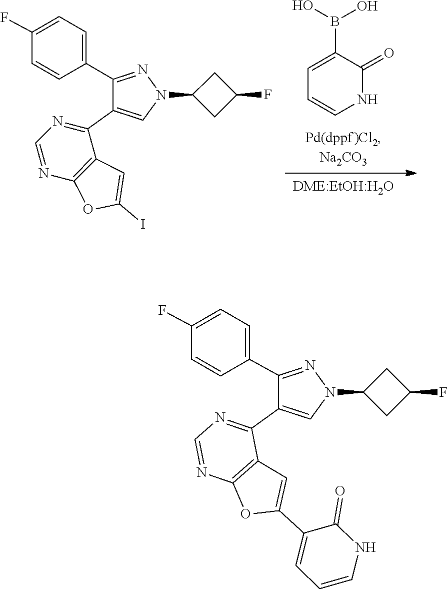

1,1′-Bis(diphenylphosphino)ferrocene dichloropalladium (II) (24.2 mg, 31.4 μmol, 0.1 equiv.) was added to a degassed mixture of 4-(1-((1s,3s)-3-fluorocyclobutyl)-3-(4-fluorophenyl)-1H-pyrazol-4-yl)-6-iodofuro[2,3-d]pyrimidine (Intermediate AA, 150 mg, 314 μmol, 1.0 equiv.), (2-oxo-1,2-dihydropyridin-3-yl)boronic acid (47.9 mg, 345 μmol, 1.1 equiv.) and sodium carbonate (101 mg, 941 mol, 3.0 equiv.) in a mixture of DME (2.03 mL), EtOH (1.02 mL) and H₂ (509 μL). The mixture was stirred at 85 HC for 16 hours. The mixture was cooled to room temperature and diluted with water and the aqueous layer was extracted with EtOAc (3×20 mL). The combined organic layers were washed with brine, then dried (Na₂SO₄), filtered and concentrated under reduced pressure. The crude material was purified by reverse phase chromatography (C18 column, MeCN in water with 10 mM ammonium formate, 20-100%, a gradient elution) to provide title compound (32.8 mg, 23H) as a solid. ¹HNMR (400 MHz, DMSO-d₆) δ 12.26 (s, 1H), 8.79 (s, 1H), 8.60 (s, 1H), 8.14 (dd, J=7.2, 2.0 Hz, 1H), 7.60-7.53 (m, 3H), 7.40 (s, 1H), 7.20-7.13 (m, 2H), 6.44 (t, J=6.8 Hz, 1H), 5.06 (dp, J=56.7, 6.8 Hz, 1H), 4.75-4.63 (i, 1H), 3.06-2.95 (m, 2H), 2.95-2.77 (m, 2H). m/z (ESI, +ve ion)=446.2 [M+H]⁺.

The compounds in Table 2 were prepared using materials and methods analogous to those disclosed herein and methods known to those having ordinary skill in the art.

TABLE 2

| Example | | |
|---|---|---|
| No. | Name | Analytical Data |
| 1 | 4-[1-(4-fluorophenyl)-1H- | ¹H-NMR (400 MHz, Chloroform-d) d 12.51 (1H, |
| | pyrazol-5-yl]-2-phenyl-1H- | s), 8.19 (1H, d, J = 5.0 Hz), 7.86 (1H, d, J = 1.5 |
| | pyrrolo[2,3-b]pyridine | Hz), 7.81 (2H, d, J = 7.8 Hz), (7.51 (2H), 7.41 |
| | | (1H), 7.33 (2H, m), 7.01 (2H), 6.82 (1H, d, J = |
| | | 1.3 Hz), 6.78 (1H, d, J = 5.1 Hz), 6.69 (1H, s). |
| | | m/z (ESI, +ve ion) = 355.1 [M + H]⁺. |
| 20 | 1-[3-(4-fluorophenyl)-4-(2- | ¹H NMR (400 MHz, DMSO-d6) δ 8.94 (s, 1H), |
| | phenyl[1,3]oxazolo[5,4- | 8.78 (s, 1H), 8.28-8.23 (m, 2H), 7.75-7.61 (m, |
| | d]pyrimidin-7-yl)-1H- | 5H), 7.20 (t, J = 8.9 Hz, 2H), 4.53 (t, J = 6.7 Hz, |
| | pyrazol-1-yl]pentan-3-one | 2H), 3.17 (t, J = 6.7 Hz, 2H), 2.57-2.51 (m, |
| | | 2H), 0.95 (t, J = 7.3 Hz, 3H). m/z (ESI, +ve |
| | | ion) = 442.3 [M + H]⁺. |
| 22 | 4-[5-(4-fluorophenyl)-4-(2- | ¹H NMR (400 MHz, DMSO-d₆) δ 8.68 (s, 1H), |
| | phenyl[1,3]oxazolo[5,4- | 8.66 (s, 1H), 8.21 (dd, J = 8.3, 1.3 Hz, 2H), 7.75- |
| | d]pyrimidin-7-yl)-1H- | 7.62 (m, 4H), 7.55 (dd, J = 8.8, 5.5 Hz, 2H), |
| | pyrazol-1-yl]-2-methylbutan- | 7.35 (t, J = 8.9 Hz, 2H), 4.38 (s, 1H), 4.15-4.04 |
| | 2-ol | (m, 2H), 1.91-1.76 (m, 2H), 1.02 (s, 6H). m/z |
| | | (ESI, +ve ion) = 444.3 [M + H]⁺. |
| 24 | 1-{2-[3-(4-fluorophenyl)-4- | ¹H NMR (400 MHz, DMSO-d₆) δ 8.76 (s, 1H), |
| | (6-phenylfuro[2,3- | 8.64 (s, 1H), 7.93 (d, J = 7.9 Hz, 2H), 7.66-7.53 |
| | d]pyrimidin-4-yl)-1H- | (m, 4H), 7.53-7.47 (m, 1H), 7.45 (d, J = 1.3 Hz, |
| | pyrazol-1- | 1H), 7.19 (t, J = 8.0 Hz, 2H), 4.71 (t, J = 4.4 Hz, |
| | yl]ethyl}cyclopropan-1-ol | 1H), 4.34 (t, J = 6.9 Hz, 2H), 3.56-3.43 (m, |
| | | 2H), 2.15-2.00 (m, 2H). m/z (ESI, +ve ion) = |
| | | 441.2 [M + H]⁺ |
| 25 | [3-(4-fluorophenyl)-4-(2- | ¹H NMR (400 MHz, DMSO-d₆) δ 9.11 (s, 1H), |
| | phenyl[1,3]oxazolo[5,4- | 8.82 (s, 1H), 8.29-8.24 (m, 2H), 7.75-7.65 (m, |
| | d]pyrimidin-7-yl)-1H- | 5H), 7.23 (t, J = 8.9 Hz, 2H), 5.76 (s, 2H). m/z |
| | pyrazol-1-yl]acetonitrile | (ESI, +ve ion) = 397.2 [M + H]⁺ |
| 26 | 7-[1-(4,4-difluorobutyl)-3-(4- | ¹H NMR (400 MHz, DMSO-d₆) δ 8.97 (s, 1H), |
| | fluorophenyl)-1H-pyrazol-4- | 8.79 (s, 1H), 8.29-8.23 (m, 2H), 7.75-7.64 (m, |
| | yl]-2-phenyl[1,3]oxazolo[5,4- | 5H), 7.21 (t, J = 8.9 Hz, 2H), 6.17 (tt, J = 56.8, |
| | d]pyrimidine | 4.2 Hz, 1H), 4.41 (t, J = 6.8 Hz, 2H), 2.10-2.00 |
| | | (m, 2H), 1.99-1.81 (m, 2H). m/z (ESI, +ve |
| | | ion) = 450.3 [M + H]⁺. |
| 28 | 4-[4-(4-fluorophenyl)-5-(8- | m/z (ESI +ve ion) = 430.15 [M + H]⁺. ¹H NMR |
| | phenyl-9H-purin-6-yl)-2H- | (400 MHz, Methanol-d₄) δ 8.88 (s, 1H), 8.19- |
| | 1,2,3-triazol-2-yl]butan-1-ol | 8.17 (m, 2H), 7.81-7.77 (m, 2H), 7.58-7.56 (m, |
| | | 3H), 7.14-7.09 (m, 2H), 4.73-4.69 (m, 2H), |
| | | 3.68-3.65 (m, 2H), 2.27-2.19 (m, 2H), 1.75- |
| | | 1.68 (m, 2H). |
| 30 | [3-(4-fluorophenyl)-4-(6- | ¹H NMR (400 MHz, DMSO-d₆) δ 8.82 (s, 1H), |
| | phenylfuro[2,3-d]pyrimidin- | 8.71 (s, 1H), 7.93-7.88 (m, 2H), 7.64-7.47 (m, |
| | 4-yl)-1H-pyrazol-1- | 5H), 7.37 (s, 1H), 7.25-7.18 (m, 2H), 5.67 (s, |
| | yllacetonitrile | 2H). m/z (ESI, +ve ion) = 396.2 [M + H]⁺. |
| 32 | 4-{1-[(3-fluorooxetan-3- | ¹H NMR (400 MHz, DMSO-d₆) δ 8.79 (s, 1H), |
| | yl)methyl]-3-(4- | 8.68 (s, 1H), 7.93-7.87 (m, 2H), 7.64-7.52 (m, |
| | fluorophenyl)-1H-pyrazol-4- | 4H), 7.49 (t, J = 7.4 Hz, 1H), 7.32 (s, 1H), 7.20 |
| | yl}-6-phenylfuro[2,3- | (t, J = 8.9 Hz, 2H), 4.97 (d, J = 8.5 Hz, 1H), |
| | d]pyrimidine | 4.94-4.84 (m, 3H), 4.76 (d, J = 8.3 Hz, 1H), 4.71 |
| | | (d, J = 8.4 Hz, 1H). m/z (ESI, +ve ion) = 445.2 |
| | | [M + H]⁺. |
| 34 | 4-[3-(4-fluorophenyl)-4-(6- | ¹H NMR (400 MHz, DMSO-d₆) δ 8.75 (s, 1H), |
| | phenylfuro[2,3-d]pyrimidin- | 8.66 (s, 1H), 7.96-7.91 (m, 2H), 7.64-7.58 (m, |
| | 4-yl)-1H-pyrazol-1-yl]-2- | 2H), 7.58-7.53 (m, 2H), 7.52-7.48 (m, 1H), |
| | methylbutan-2-ol | 7.47 (s, 1H), 7.22-7.15 (m, 2H), 4.54 (s, 1H), |
| | | 4.42-4.32 (m, 2H), 2.12-2.02 (m, 2H), 1.20 (s, |
| | | 6H). m/z (ESI, +ve ion) = 443.2 [M + H]⁺. |
| 36 | 3-[4-(4-fluorophenyl)-5-(6- | m/z (ESI, +ve ion) = 443.15 [M + H]⁺. ¹H-NMR |
| | phenylfuro[2,3-d]pyrimidin- | (400 MHz, Chloroform-d) δ 8.90 (s, 1H), 8.24- |
| | 4-yl)-1,3-oxazol-2-yl]-N- | 8.19 (m, 2H), 8.03-7.97 (m, 2H), 7.58-7.43 (m, |
| | methylpropanamide | 3H) 7.39 (s, 1H), 7.32-7.19 (m, 2H), 5.7 (s, 1H), |
| | | 3.66-3.39 (m, 2H), 2.96-2.82 (m, 5H) |
| 37 | 3-[4-(4-fluorophenyl)-5-(6- | m/z (ESI, +ve ion) = 457.15 [M + H]⁺. ¹H NMR |
| | phenylfuro[2,3-d]pyrimidin- | (400 MHz, Chloroform-d) δ 8.90-8.89 (d, J = |
| | 4-yl)-1,3-oxazol-2-yl]-N,N- | 5.6Hz, 1H), 8.25-8.21 (m, 2H), 8.01-7.99 (m, |
| | dimethylpropanamide | 2H), 7.56-7.46 (m, 3H), 7.43 (s, 1H), 7.19-7.15 |
| | | (m, 2H), 3.44-3.40 (m, 2H), 3.12-3.08 (d, J = |
| | | 13.6 Hz, 3H), 3.03-3.00 (m, 5H) |
| 39 | 4-[3-(4-fluorophenyl)-1- | m/z (ESI, +ve ion) = 435.00 [M + H]⁺. ¹H NMR |
| | (methanesulfonyl)-1H- | (400 MHz, DMSO-d₆) δ 8.97 (s, 1H), 8.86 (s, |
| | pyrazol-4-yl]-6- | 1H), 8.02-7.95 (m, 2H), 7.69-7.60 (m, 3H), |
| | phenylfuro[2,3-d]pyrimidine | 7.62-7.53 (m, 2H), 7.56-7.47 (m, 1H), 7.30- |
| | | 7.19 (m, 2H), 3.79 (s, 3H) |
| 41 | 3-[3-(4-fluorophenyl)-4-(6- | m/z (ESI, +ve ion) = 475.15 [M + H]⁺. ¹H NMR |
| | phenylfuro[2,3-d]pyrimidin- | (400 MHz, Chloroform-d) δ 8.94 (s, 1H), 8.18 (s, |
| | 4-yl)-1H-pyrazol-1-yl]-1λ6- | 1H), 7.67-7.65 (m, 2H), 7.57-7.53 (m, 2H), |
| | thiolane-1,1-dione | 7.48-7.40 (m, 3H), 7.13-7.08 (m, 2H), 6.12 (s, |
| | | 1H), 5.28-5.21 (m, 1H), 3.85-3.79 (m, 1H), |
| | | 3.74-3.60 (m, 2H), 3.34-3.27 (m, 1H), 2.98- |
| | | 2.85 (m, 2H) |
| 44 | 4-[4-(4-fluorophenyl)-5-(6- | m/z (ESI, +ve ion) = 444.30 [M + H]⁺. ¹H-NMR |
| | phenylfuro[2,3-d]pyrimidin- | (400 MHz, Chloroform-d) δ 9.03 (s, 1H), 8.18- |
| | 4-yl)-1,3-oxazol-2-yl]-2- | 8.14 (m, 2H), 7.93 (d, J = 7.2 Hz, 2H), 7.55- |
| | methylbutan-2-ol | 7.46 (m, 3H), 7.22-7.14 (m, 3H), 3.21-3.17 (m, |
| | | 2H), 2.20-2.14 (m, 2H), 1.39 (s, 6H) |
| 49 | 3-[4-(4-fluorophenyl)-5-(6- | m/z (ESI, +ve ion) = 429.15 [M + H]⁺. ¹H NMR |
| | phenylfuro[2,3-d]pyrimidin- | (400 MHz, Chloroform-d) δ 8.91 (s, 1H), 8.23- |
| | 4-yl)-1,3-oxazol-2- | 8.20 (m, 2H), 8.00-7.98 (m, 2H), 7.56-7.53 (m, |
| | yl]propanamide | 2H), 7.50-7.46 (m, 1H), 7.37 (s, 1H), 7.20-7.15 |
| | | (m, 2H), 5.78 (s, 1H), 5.43 (s, 1H), 3.43-3.39 |
| | | (m, 2H), 2.94-2.91 (m, 2H) |
| 50 | 2-[3-(4-fluorophenyl)-4-(6- | m/z (ESI, +ve ion) = 492.30 [M + H]⁺. ¹H-NMR |
| | phenylfuro[2,3-d]pyrimidin- | (400 MHz, DMSO-d₆) δ 8.77 (d, J = 9.6 Hz, 2H), |
| | 4-yl)-1H-pyrazol-1-yl]-N,N- | 7.92 (d, J = 7.2 Hz, 2H), 7.65-7.51 (m, 6H), |
| | dimethylethane-1- | 7.21 (t, J = 8.8 Hz, 2H), 4.69 (t, J = 7.2 Hz, 2H), |
| | sulfonamide | 3.78 (t, J = 7.2 Hz, 2H), 2.81 (s, 6H) |
| 51 | 3-(4-fluorophenyl)-N,N- | m/z (ESI, +ve ion) = 464.15 [M + H]⁺. ¹H NMR |
| | dimethyl-4-(6- | (400 MHz, DMSO-d₆) δ 8.96 (s, 1H), 8.84 (s, |
| | phenylfuro[2,3-d]pyrimidin- | 1H), 8.03-7.96 (m, 2H), 7.65-7.55 (m, 4H), |
| | 4-yl)-1H-pyrazole-1- | 7.58-7.46 (m, 2H), 7.29-7.19 (m, 2H), 3.03 (s, |
| | sulfonamide | 6H) |
| 53 | 3-{[3-(4-fluorophenyl)-4-(6- | m/z (ESI, +ve ion) = 489.15 [M + H]⁺. ¹H NMR |
| | phenylfuro[2,3-d]pyrimidin- | (400 MHz, Chloroform-d) δ 8.80 (s, 1H), 8.27 (s, |
| | 4-yl)-1H-pyrazol-1- | 1H), 7.76-7.73 (m, 2H), 7.51-7.41 (m, 5H), |
| | yl]methyl}-1λ6-thiolane-1,1- | 7.28-7.24 (m, 2H), 6.36 (s, 1H), 4.30-4.28 (d, J = |
| | dione | 7.12 Hz, 2H), 3.24-3.02 (m, 4H), 2.85-2.79 |
| | | (m, 1H), 2.28-2.26 (m, 1H), 1.92-1.87 (m, 1H) |
| 54 | (1r,3r)-3-{[5-(4- | m/z (ESI, +ve ion) = 441.30 [M + H]⁺. ¹H NMR |
| | fluorophenyl)-4-(6- | (400 MHz, Chloroform-d) δ 8.77 (s, 1H), 8.21 (s, |
| | phenylfuro[2,3-d]pyrimidin- | 1H), 7.76-7.69 (m, 2H), 7.52-7.38 (m, 5H), |
| | 4-yl)-1H-pyrazol-1- | 7.28-7.18 (m, 2H), 6.34 (s, 1H), 4.36-4.29 (m, |
| | yl]methyl}cyclobutan-1-ol | 1H), 4.21 (d, J = 8.0 Hz, 2H), 2.81-2.70 (m, |
| | | 1H), 2.22-2.12 (m, 2H), 2.10-1.98 (m, 2H) |
| 56 | 4-[5-(4-fluorophenyl)-1- | m/z (ESI, + ve ion) = 435.10 [M + H]⁺. ¹H NMR |
| | (methanesulfonyl)-1H- | (400 MHz, DMSO-d₆) δ 8.77 (s, 1H), 8.70 (s, |
| | pyrazol-4-yl]-6- | 1H), 7.91-7.84 (m, 2H), 7.64-7.45 (m, 5H), |
| | phenylfuro[2,3-d]pyrimidine | 7.33-7.24 (m, 2H), 7.14 (s, 1H), 3.64 (s, 3H) |
| 57 | 4-[4-(4-fluorophenyl)-5-(2- | m/z (ESI, +ve ion) = 445.15 [M + H]⁺. ¹H-NMR |
| | phenyl[1,3]oxazolo[5,4- | (400 MHz, DMSO-d₆) δ 8.99 (s, 1H), 8.18-8.16 |
| | d]pyrimidin-7-yl)-1,3-oxazol- | (m, 2H), 8.09-8.04 (m, 2H), 7.75-7.64 (m, 3H), |
| | 2-yl]-2-methylbutan-2-ol | 7.29-7.23 (m, 2H), 4.44 (s, 1H), 3.07-3.03 (m, |
| | | 2H), 2.03-1.19 (m, 2H), 1.21 (s, 6H) |
| 59 | 4-{3-(4-fluorophenyl)-1-[(3- | ¹H NMR (400 MHz, DMSO-d₆) δ 8.78 (s, 1H), |
| | methyloxetan-3-yl)methyl]- | 8.69 (s, 1H), 7.96-7.84 (m, 2H), 7.60 (dd, J = |
| | 1H-pyrazol-4-yl}-6- | 8.0, 5.3 Hz, 2H), 7.55 (d, J = 7.7 Hz, 2H), 7.49 (t, |
| | phenylfuro[2,3-d]pyrimidine | J = 7.3 Hz, 1H), 7.35 (s, 1H), 7.19 (t, J = 8.8 Hz, |
| | | 2H), 4.71 (d, J = 5.9 Hz, 2H), 4.51 (s, 2H), 4.32 |
| | | (d, J = 6.0 H, 2Hz), 1.29 (s, 3H). m/z (ESI, +ve |
| | | ion) = 441.5 [M + H]⁺ |
| 66 | 4-[4-(4-fluorophenyl)-5-(6- | m/z (ESI, +ve ion) = 460.15 [M + H]⁺. ¹H-NMR |
| | phenylthieno[2,3- | (400 MHz, DMSO-d₆) δ 9.01 (s, 1H), 8.12-8.07 |
| | d]pyrimidin-4-yl)-1,3-oxazol- | (m, 3H), 7.86-7.84 (m, 2H), 7.57-7.48 (m, 3H), |
| | 2-yl]-2-methylbutan-2-ol | 7.29-7.25 (m, 2H), 4.45 (s, 1H), 3.10-3.07 (m, |
| | | 2H), 2.03-1.99 (m, 2H), 1.21 (s, 6H) |
| 67 | 4-[3-(4-fluorophenyl)-4-(6- | m/z (ESI, +ve ion) = 459.15 [M + H]⁺. ¹H NMR |
| | phenylthieno[2,3- | (400 MHz, Chloroform-d) δ 9.05 (s, 1H), 8.10 (s, |
| | d]pyrimidin-4-yl)-1H- | 1H), 7.52-7.49 (m, 2H), 7.44-7.36 (m, 5H), |
| | pyrazol-1-yl]-2-methylbutan- | 7.06-7.02 (m, 2H), 6.94 (s, 1H), 4.49-4.45 (m, |
| | 2-ol | 2H), 2.25-2.21 (m, 2H), 1.37 (s, 6H) |
| 68 | 3-[3-(4-fluorophenyl)-4-(6- | m/z (ESI, +ve ion) = 431.15 [M + H]⁺. ¹H NMR |
| | phenylthieno[2,3- | (400 MHz, Chloroform-d) δ 9.04 (s, 1H), 8.02 (s, |
| | d]pyrimidin-4-yl)-1H- | 1H), 7.53-7.48 (m, 2H), 7.43-7.35 (m, 5H), |
| | pyrazol-1-yl]propan-1-ol | 7.07-7.00 (m, 2H), 6.96 (s, 1H), 4.48-4.45 (m, |
| | | 2H), 3.82-3.78 (m, 2H), 2.55-2.53 (m, 1H), |
| | | 2.26-2.20 (m, 2H) |
| 71 | 4-[1-(4-fluorobutyl)-3-(4- | ¹H NMR (400 MHz, DMSO-d₆) δ 8.77 (s, 1H), |
| | fluorophenyl)-1H-pyrazol-4- | 8.66 (s, 1H), 7.94-7.89 (m, 2H), 7.63-7.53 (m, |
| | yl]-6-phenylfuro[2,3- | 4H), 7.52-7.47 (m, 1H), 7.41 (s, 1H), 7.19 (t, J = |
| | d]pyrimidine | 8.9 Hz, 2H), 4.57 (app t, J = 6.0 Hz, 1H), 4.45 |
| | | (app t, J = 6.0 Hz, 1H), 4.32 (t, J = 7.0 Hz, 2H), |
| | | 2.07-1.98 (m, 2H), 1.81-1.65 (m, 2H). m/z |
| | | (ESI, +ve ion) = 431.5 [M + H]⁺ |
| 74 | 3-[4-(4-fluorophenyl)-5-(6- | m/z (ESI, +ve ion) = 432.10 [M + H]⁺. ¹H-NMR |
| | phenylthieno[2,3- | (400 MHz, DMSO-d₆) δ 9.01 (s, 1H), 8.11-8.07 |
| | d]pyrimidin-4-yl)-1,3-oxazol- | (m, 3H), 7.86 (d, J = 7.2 Hz, 2H), 7.57-7.48 (m, |
| | 2-yl]propan-1-ol | 3H), 7.30-7.25 (m, 2H), 4.68-4.66 (m, 1H), |
| | | 3.62-3.58 (m, 2H), 3.11-3.08 (m, 2H), 2.07- |
| | | 2.01 (m, 2H) |
| 75 | 4-{3-(4-fluorophenyl)-1-[(3- | m/z (ESI, +ve ion) = 457.15 [M + H]⁺. ¹H NMR |
| | methoxyoxetan-3-yl)methyl]- | (400 MHz, Chloroform-d) δ 8.94 (s, 1H), 8.15 (s, |
| | 1H-pyrazol-4-yl}-6- | 1H), 7.67-7.64 (m, 2H), 7.57-7.54 (m, 2H), |
| | phenylfuro[2,3-d]pyrimidine | 7.48-7.40 (m, 3H), 7.12-7.08 (m, 2H), 6.13 (s, |
| | | 1H), 4.88-4.87 (d, J = 7.36 Hz, 2H), 4.73-4.71 |
| | | (m, 4H), 3.52 (s, 3H) |
| 79 | 4-{3-(4-fluorophenyl)-1- | m/z (ESI, +ve ion) = 449.10 [M + H]⁺. ¹H NMR |
| | [(methanesulfonyl)methyl]- | (400 MHz, DMSO-d₆) δ 8.85 (s, 1H), 8.78 (s, |
| | 1H-pyrazol-4-yl}-6- | 1H), 7.89-7.87 (m, 2H), 7.66-7.62 (m, 2H), |
| | phenylfuro[2,3-d]pyrimidine | 7.57 (t, J = 7.6 Hz, 2H), 7.51 (t, J = 7.2 Hz, 1H), |
| | | 7.23-7.18 (m, 3H), 5.90 (s, 2H), 3.21 (s, 3H) |
| 80 | 7-[3-(4-fluorophenyl)-1- | m/z (ESI, +ve ion) = 414.25 [M + H]⁺. ¹H NMR |
| | (oxetan-3-yl)-1H-pyrazol-4- | (400 MHz, Chloroform-d) δ 8.92 (s, 1H), 8.83 (s, |
| | yl]-2-phenyl[1,3]oxazolo[5,4- | 1H), 8.29-8.27 (m, 2H), 7.81-7.76 (m, 2H), |
| | d]pyrimidine | 7.67-7.54 (m, 3H), 7.15-7.10 (m, 2H), 5.71- |
| | | 5.64 (m, 1H), 5.30-5.27 (m, 2H), 5.19-5.15 (m, 2H) |
| 81 | 4-[5-(4-fluorophenyl)-2- | m/z (ESI, +ve ion) = 414.15 [M + H]⁺. ¹H-NMR |
| | (oxetan-3-yl)-2H-1,2,3- | (400 MHz, Chloroform-d) δ 8.93 (s, 1H), 7.97- |
| | triazol-4-yl]-6- | 7.94 (m, 2H), 7.94-7.88 (m, 2H), 7.55-7.46 (m, |
| | phenylfuro[2,3-d]pyrimidine | 3H), 7.41 (s, 1H), 7.19-7.13 (m, 2H), 6.03-5.96 |
| | | (m, 1H), 5.41-5.38 (m, 2H), 5.25-5.21 (m, 2H) |
| 82 | 4-[4-(4-fluorophenyl)-5-(6- | m/z (ESI, +ve ion) = 444.20 [M + H]⁺. ¹H-NMR |
| | phenylfuro[2,3-d]pyrimidin- | (400 MHz, DMSO-d₆) δ 8.86 (s, 1H), 8.04 (d, J = |
| | 4-yl)-2H-1,2,3-triazol-2-yl]- | 7.6 Hz, 2H), 7.89-7.85 (m, 2H), 7.78 (s, 1H), |
| | 2-methylbutan-2-ol | 7.61-7.51 (m, 3H), 7.29-7.25 (m, 2H), 4.75- |
| | | 4.71 (m, 2H), 4.61 (s, 1H), 2.21-2.17 (m, 2H), |
| | | 1.21 (s, 6H) |
| 83 | 4-[3-(4-fluorophenyl)-4-(6- | m/z (ESI, +ve ion) = 444.20 [M + H]⁺. ¹H-NMR |
| | phenylfuro[2,3-d]pyrimidin- | (400 MHz, DMSO-d₆) δ 8.86 (s, 1H), 8.04 (d, J = |
| | 4-yl)-1H-pyrazol-1-yl]-1λ6- | 7.6 Hz, 2H), 7.89-7.85 (m, 2H), 7.78 (s, 1H), |
| | thiane-1, 1-dione | 7.61-7.51 (m, 3H), 7.29-7.25 (m, 2H), 4.75- |
| | | 4.71 (m, 2H), 4.61 (s, 1H), 2.21-2.17 (m, 2H), |
| | | 1.21 (s, 6H) |
| 84 | 3-[3-(4-fluorophenyl)-4-(6- | m/z (ESI, +ve ion) = 489.15 [M + H]⁺. ¹H-NMR |
| | phenylfuro[2,3-d]pyrimidin- | (400 MHz, DMSO-d₆) δ 8.76 (d, J = 8.4 Hz, 2H), |
| | 4-yl)-1H-pyrazol-1-yl]-1λ6- | 7.94 (d, J = 7.2 Hz, 2H), 7.65-7.56 (m, 4H), |
| | thiane-1,1-dione | 7.53-7.49 (m, 2H), 7.23-7.19 (m, 2H), 4.92- |
| | | 4.85 (m, 1H), 3.87-3.73 (m, 2H), 3.31-3.19 (m, |
| | | 2H), 2.31-2.17 (m, 3H), 2.00-1.94 (m, 1H) |
| 85 | 4-[3-(4-fluorophenyl)-1- | m/z (ESI, +ve ion) = 427.15 [M + H]⁺. ¹H NMR |
| | (oxolan-3-yl)-1H-pyrazol-4- | (400 MHz, Chloroform-d) δ 8.93 (s, 1H), 8.18 (s, |
| | yl]-6-phenylfuro[2,3- | 1H), 7.65-7.63 (m, 2H), 7.63-7.53 (m, 2H), |
| | d]pyrimidine | 7.47-7.29 (m, 3H), 7.12-7.07 (m, 2H), 6.09 (s, |
| | | 1H), 5.15-5.12 (m, 1H), 4.30-4.22 (m, 2H), |
| | | 4.30-4.15 (m, 1H), 4.06-4.00 (m, 1H), 2.61- |
| | | 2.51 (m, 2H) |
| 86 | 1-{3-[3-(4-fluorophenyl)-4- | m/z (ESI, +ve ion) = 454.30 [M + H]⁺. ¹H NMR |
| | (6-phenylfuro[2,3- | (400 MHz, Chloroform-d) δ 8.93 (s, 1H), 8.18 (s, |
| | d]pyrimidin-4-yl)-1H- | 1H), 7.66-7.64 (m, 2H), 7.59-7.55 (m, 2H), |
| | pyrazol-1-yl]azetidin-1- | 7.48-7.40 (m, 3H), 7.14-7.09 (m, 2H), 6.10 (s, |
| | yl}ethan-1-one | 1H), 5.29-5.22 (m, 1H), 4.77-4.74 (m, 1H), |
| | | 4.69-4.64 (m, 1H), 4.60-4.56 (m, 2H), 2.00 (s, 3H) |
| 87 | 4-{5-(4-fluorophenyl)-2-[2- | m/z (ESI, +ve ion) = 464.10 [M + H]⁺. ¹H-NMR |
| | (methanesulfonyl)ethyl]-2H- | (400 MHz, Chloroform-d) δ 8.91 (s, 1H), 7.98- |
| | 1,2,3-triazol-4-yl}-6- | 7.96 (m, 2H), 7.91-7.85 (m, 2H), 7.56-7.46 (m, |
| | phenylfuro[2,3-d]pyrimidine | 3H), 7.41 (s, 1H), 7.18-7.13 (m, 2H), 5.18-5.15 |
| | | (m, 2H), 3.91-3.88 (m, 2H), 2.96 (s, 3H) |
| 88 | 1-[4-(4-fluorophenyl)-5-(6- | m/z (ESI, +ve ion) = 430.20 [M + H]⁺. ¹H-NMR |
| | phenylfuro[2,3-d]pyrimidin- | (400 MHz, Chloroform-d) δ 8.92 (s, 1H), 7.99- |
| | 4-yl)-2H-1,2,3-triazol-2-yl]- | 7.87 (m, 4H), 7.57-7.43 (m, 3H), 7.39 (s, 1H), |
| | 2-methylpropan-2-ol | 7.21-7.11 (m, 2H), 4.65 (s, 2H), 1.38 (s, 6H) |
| 89 | 4-{3-(4-fluorophenyl)-1-[1- | m/z (ESI, +ve ion) = 504.30 [M + H]⁺. ¹H NMR |
| | (methanesulfonyl)pyrrolidin- | (400 MHz, DMSO-d₆) δ 8.79 (s, 1H), 8.70 (s, |
| | 3-yl]-1H-pyrazol-4-yl}-6- | 1H), 7.92-7.90 (m, 2H), 7.64-7.55 (m, 4H), |
| | phenylfuro[2,3-d]pyrimidine | 7.52-7.48 (m, 1H), 7.39 (s, 1H), 7.23-7.18 (m, |
| | | 2H), 5.26-5.20 (m, 1H), 3.90-3.87 (m, 1H), |
| | | 3.80-3.76 (m, 1H), 3.62-3.59 (m, 1H), 3.55- |
| | | 3.49 (m, 1H), 2.99 (s, 3H), 2.58-2.50 (m, 2H) |
| 92 | 1-{3-[3-(4-fluorophenyl)-4- | m/z (ESI, +ve ion) = 468.30 [M + H]⁺. ¹H NMR |
| | (6-phenylfuro[2,3- | (400 MHz, Chloroform-d) δ 8.80-8.69 (m, 1H), |
| | d]pyrimidin-4-yl)-1H- | 8.44-8.31 (m, 1H), 7.78-7.77 (m, 2H), 7.58- |
| | pyrazol-1-yl]pyrrolidin-1- | 7.32 (m, 5H), 7.15-7.10 (m, 2H), 6.68-6.65 (m, |
| | yl}ethan-1-one | 1H), 4.84-4.82 (m, 1H), 4.04-3.32 (m, 4H), |
| | | 2.66-2.15 (m, 2H), 2.14-2.13 (d, J = 7.84 Hz, 3H) |
| 93 | 4-[3-(4-fluorophenyl)-1-(2- | ¹H NMR (400 MHz, DMSO-6) δ 8.79 (s, 1H), |
| | methoxy-2-methylpropyl)- | 8.48 (s, 1H), 7.88 (d, J = 7.2 Hz, 2H), 7.63-7.53 |
| | 1H-pyrazol-4-yl]-6- | (m, 4H), 7.52-7.45 (m, 1H), 7.24 (s, 1H), 7.19 |
| | phenylfuro[2,3-d]pyrimidine | (app t, J = 8.9 Hz, 2H), 4.30 (s, 2H), 3.24 (s, 3H), |
| | | 1.21 (s, 6H). m/z (ESI, +ve ion) = 443.5 [M + H]⁺ |
| 98 | 3-{[3-(4-fluorophenyl)-4-(6- | m/z (ESI, +ve ion) = 475.25 [M + H]⁺. ¹H NMR |
| | phenylfuro[2,3-d]pyrimidin- | (400 MHz, Chloroform-d) δ 8.94 (s, 1H), 8.13 (s, |
| | 4-yl)-1H-pyrazol-1- | 1H), 7.66-7.65 (m, 2H), 7.55-7.52 (m, 2H), |
| | yl]methyl}-1λ6-thietane-1,1- | 7.47-7.42 (m, 3H), 7.12-7.08 (m, 2H), 6.44 (s, |
| | dione | 1H), 4.57-4.55 (d, J = 7.6 Hz, 2H), 4.38-4.33 |
| | | (m,2H), 4.12-4.07 (m, 2H), 3.51 (s, 1H) |
| 99 | (3R)-3-[3-(4-fluorophenyl)-4- | m/z (ESI, +ve ion) = 489.15 [M + H]⁺. ¹H-NMR |
| | (6-phenylfuro[2,3- | (400 MHz, Chloroform-d) δ 8.93 (s, 1H), 8.16 (s, |
| | d]pyrimidin-4-yl)-1H- | 1H), 7.67-7.64 (m, 2H), 7.55-7.51 (m, 2H), |
| | pyrazol-1-yl]-1λ6-thiane-1,1- | 7.48-7.39 (m, 3H), 7.12-7.08 (m, 2H), 6.11 (s, |
| | dione | 1H), 4.88-4.87 (m, 1H), 3.76-3.69 (m, 1H), |
| | (absolute stereochemistry | 3.62-3.58 (m, 1H), 3.23-3.18 (m, 1H), 3.09- |
| | arbitrarily assigned) | 3.05 (m, 1H), 2.44 (d, J = 8.4 Hz, 1H), 2.36- |
| | | 2.02 (m, 3H) |
| 100 | (3S)-3-[3-(4-fluorophenyl)-4- | m/z (ESI, +ve ion) = 489.15 [M + H]⁺. ¹H-NMR |
| | (6-phenylfuro[2,3- | (400 MHz, Chloroform-d) δ 8.93 (s, 1H), 8.16 (s, |
| | d]pyrimidin-4-yl)-1H- | 1H), 7.67-7.64 (m, 2H), 7.55-7.51 (m, 2H), |
| | pyrazol-1-yl]-1λ6-thiane-1,1- | 7.48-7.39 (m, 3H), 7.12-7.08 (m, 2H), 6.11 (s, |
| | dione | 1H), 4.88-4.87 (m, 1H), 3.76-3.69 (m, 1H), |
| | (absolute stereochemistry | 3.62-3.58 (m, 1H), 3.23-3.18 (m, 1H), 3.09- |
| | arbitrarily assigned) | 3.05 (m, 1H), 2.44 (d, J = 8.4 Hz, 1H), 2.36- |
| | | 2.02 (m, 3H) |
| 105 | 4-{[3-(4-fluorophenyl)-4-(6- | [00286] m/z (ESI, +ve ion) = 503.30 [M + |
| | phenylfuro[2,3-d]pyrimidin- | H]⁺. ¹H-NMR (400 MHz, Chloroform-d) δ |
| | 4-yl)-1H-pyrazol-1- | 8.97 (s, 1H), 8.26 (s, 1H), 7.65-7.63 (m, 2H), |
| | yl]methyl}-1λ6-thiane-1,1- | 7.56-7.52 (m, 2H), 7.47-7.41 (m, 3H), 7.13- |
| | dione | 7.09 (m, 2H), 6.07 (s, 1H), 4.21 (d, J = 7.2 Hz, |
| | | 2H), 3.14-2.97 (m, 4H), 2.45-2.32 (m, 1H), |
| | | 2.18-1.96 (m, 5H). |
| 107 | 4-[1-(difluoromethyl)-3-(4- | ¹H NMR (400 MHz, DMSO-d₆) δ 9.06 (s, 1), |
| | fluorophenyl)-1H-pyrazol-4- | 8.84 (s, 1H), 8.14-7.82 (m, 3H), 7.65-7.55 (m, |
| | yl]-6-phenylfuro[2,3- | 4H), 7.54 (s, 1H), 7.50 (t, J = 7.3 Hz, 1H), 7.23 |
| | d]pyrimidine | (t, J = 8.9 Hz, 2H). m/z (ESI, +ve ion) = 407.4 |
| | | [M + H]⁺ |
| 112 | 3-((5-(4-fluorophenyl)-4-(6- | m/z (ESI, +ve ion) = 475.20 [M + H]⁺. ¹H NMR |
| | phenylfuro[2,3-d]pyrimidin- | (400 MHz, Chloroform-d) δ 8.81 (s, 1H), 8.26 (s, |
| | 4-yl)-1H-pyrazol-1- | 1H), 7.75-7.73 (m, 2H), 7.55-7.42 (m, 5H), |
| | yl)methyl)thietane 1,1- | 7.28-7.25 (m, 2H), 6.34 (s, 1H), 4.45-4.44 (d, J = |
| | dioxide | 7.76 Hz, 2H), 4.27-4.23 (m, 2H), 3.96-3.91 |
| | | (m, 2H), 3.18-3.14 (m, 1H) |
| 113 | 4-{3-(4-fluorophenyl)-1-[2- | m/z (ESI, +ve ion) = 471.30 [M + H]⁺ ¹H NMR |
| | (3-methoxyoxetan-3- | (400 MHz, Chloroform-d) δ 8.91 (s, 1H), 8.06 (s, |
| | yl)ethyl]-1H-pyrazol-4-yl}-6- | 1H), 7.67-7.65 (m, 2H), 7.58-7.56 (m, 2H), |
| | phenylfuro[2,3-d]pyrimidine | 7.55-7.39 (m, 3H), 7.12-7.08 (m, 2H), 6.12 (s, |
| | | 1H), 4.74 (d, J = 7.2 Hz, 2H), 4.35-4.31 (m, |
| | | 4H), 3.41 (s, 3H), 2.66 (t, J = 7.2 Hz, 2H) |
| 114 | 4-{3-(4-fluorophenyl)-1-[1- | m/z (ESI, +ve ion) = 490.25 [M + H]⁺. ¹H NMR |
| | (methanesulfonyl)azetidin-3- | (400 MHz, DMSO-d₆) δ 8.81 (s, 1H), 8.74 (s, |
| | yl]-1H-pyrazol-4-yl}-6- | 1H), 7.93-7.91 (m, 2H), 7.69-7.65 (m, 2H), |
| | phenylfuro[2,3-d]pyrimidine | 7.60-7.56 (m, 2H), 7.53-7.51 (m, 1H), 7.43 (s, |
| | | 1H), 7.24-7.20 (m, 2H), 5.49-5.42 (m, 1H), |
| | | 4.53-4.49 (m, 2H), 4.43-4.39 (m, 2H), 3.21 (s, 3H) |
| 115 | 3-{[3-(4-fluorophenyl)-4-(6- | m/z (ESI, +ve ion) = 503.30 [M + H]⁺. ¹H-NMR |
| | phenylfuro[2,3-d]pyrimidin- | (400 MHz, Chloroform-d) δ 8.94 (s, 1H), 8.13 (s, |
| | 4-yl)-1H-pyrazol-1- | 1H), 7.67-7.64 (m, 2H), 7.56-7.52 (m, 2H), |
| | yl]methyl}-1λ6-thiane-1,1- | 7.48-7.42 (m, 3H), 7.13-7.08 (m, 2H), 6.11 (s, |
| | dione | 1H), 4.32-4.31 (m, 2H), 3.21-3.18 (m, 1H), |
| | | 3.13-3.08 (m, 1H), 2.99-2.91 (m, 3H), 2.24- |
| | | 2.15 (m, 2H), 2.01 (d, J = 16 Hz, 1H), 1.43-1.40 |
| | | (m, 1H) |
| 117 | 3-[3-(4-fluorophenyl)-4-(6- | ¹H NMR (400 MHz, DMSO-d₆) δ 8.76 (s, 1H), |
| | phenylfuro[2,3-d]pyrimidin- | 8.60 (s, 1H), 8.00-7.95 (m, 1H), 7.94-7.88 (m, |
| | 4-yl)-1H-pyrazol-1-yl]-N- | 2H), 7.63-7.53 (m, 4H), 7.53-7.45 (m, 1H), |
| | methylpropanamide | 7.42 (s, 1H), 7.22-7.14 (m, 2H), 4.49 (t, J = 7.1 |
| | | Hz, 2H), 2.80 (t, J = 7.1 Hz, 2H), 2.59 (d, J = 4.6 |
| | | Hz, 3Hz). m/z (ESI, +ve ion) = 442.3 [M + H]⁺ |
| 122 | 4-[3-(4-fluorophenyl)-1- | m/z (ESI, +ve ion) = 441.25 [M + H] +. ¹H NMR |
| | (oxan-3-yl)-1H-pyrazol-4-yl]- | (400 MHz, Chloroform-d) δ 8.93 (s, 1H), 8.23 (s, |
| | 6-phenylfuro[2,3- | 1H), 7.65-7.63 (m, 2H), 7.57-7.54 (m, 2H), |
| | d]pyrimidine | 7.47-7.41 (m, 3H), 7.12-7.01 (m, 2H), 6.10 (s, |
| | | 1H), 4.49-4.45 (m, 1H), 4.28-4.24 (m, 1H), |
| | | 3.96-3.90 (m, 2H), 3.69-3.63 (m, 1H), 2.36- |
| | | 2.29 (m, 2H), 1.91-1.82 (m, 2H) |
| 123 | 4-{1-[(3,3- | m/z (ESI, +ve ion) = 461.25 [M + H]⁺. ¹H NMR |
| | difluorocyclobutyl)methyl]-3- | (400 MHz, Chloroform-d) δ 8.92 (s, 1H), 8.06 (s, |
| | (4-fluorophenyl)-1H-pyrazol- | 1H), 7.66-7.63 (m, 2H), 7.56-7.53 (m, 2H), |
| | 4-yl}-6-phenylfuro[2,3- | 7.47-7.39 (m, 3H), 7.13-7.08 (m, 2H), 6.09 (s, |
| | d]pyrimidine | 1H), 4.39-4.37 (d, J = 6.88 Hz, 2H), 2.91-2.79 |
| | | (m, 3H), 2.58-2.47 (m, 2H) |
| 124 | 4-[1-{[(1r,3r)-3- | m/z (ESI, +ve ion) = 443.20 [M + H]⁺. ¹H NMR |
| | fluorocyclobutyl]methyl}-3- | (400 MHz, Chloroform-d) δ 8.91 (s, 1H), 8.16 (d, |
| | (4-fluorophenyl)-1H-pyrazol- | J = 1.2 Hz, 1H), 7.70-7.61 (m, 2H), 7.61-7.51 |
| | 4-yl]-6-phenylfuro[2,3- | (m, 2H), 7.49-7.36 (m, 3H), 7.14-7.04 (m, 2H), |
| | d]pyrimidine | 6.19 (s, 1H), 4.69-4.44 (m, 2H), 4.38-4.31 (m, |
| | | 1H), 1.19-1.13 (m, 1H), 0.77-0.71 (m, 2H), |
| | | 0.65-0.58 (m, 1H), 0.53-0.41 (m, 1H) |
| 128 | 1-{(3R)-3-[3-(4- | m/z (ESI, +ve ion) = 468.20 [M + H]⁺. ¹H NMR |
| | fluorophenyl)-4-(6- | (400 MHz, Chloroform-d) δ 8.93 (s, 1H), 8.15- |
| | phenylfuro[2,3-d]pyrimidin- | 8.12 (d, J = 10.4 Hz, 1H), 7.66-7.63 (m, 2H), |
| | 4-yl)-1H-pyrazol-1- | 7.56-7.52 (m, 2H), 7.48-7.41 (m, 3H), 7.16- |
| | yl]pyrrolidin-1-yl}ethan-1- | 7.07 (m, 2H), 6.11-6.07 (d, J = 17.2 Hz, 1H), |
| | one | 5.12-5.04 (m, 1H), 4.14-4.04 (m, 2H), 3.91- |
| | (absolute stereochemistry | 3.72 (m, 2H), 2.73-2.54 (m, 2H), 2.16-2.14 (m, |
| | arbitrarily assigned) | J = 7.2 Hz, 3H) |
| 129 | 1-{(3S)-3-[3-(4- | m/z (ESI, +ve ion) = 468.20 [M + H]⁺. ¹H NMR |
| | fluorophenyl)-4-(6- | (400 MHz, Chloroform-d) δ 8.92 (s, 1H), 8.12- |
| | phenylfuro[2,3-d]pyrimidin- | 8.09 (d, J = 13.5 Hz, 1H), 7.66-7.63 (m, 2H), |
| | 4-yl)-1H-pyrazol-1- | 7.56-7.52 (m, 2H), 7.47-7.41 (m, 3H), 7.12- |
| | yl]pyrrolidin-1-yl}ethan-1- | 7.07 (m, 2H), 6.12-6.07 (d, J = 18.9 Hz, 1H), |
| | one | 5.10-5.04 (m, 1H), 4.14-4.04 (m, 2H), 3.91- |
| | (absolute stereochemistry | 3.86 (m, 1H), 3.74-3.71 (m, 1H), 2.62-2.54 (m, |
| | arbitrarily assigned) | 2H), 2.15-2.14 (m, J = 6.8 Hz, 3H) |
| 131 | 6-{3-(4-fluorophenyl)-1-[2- | m/z (ESI, +ve ion) = 463.10 [M + H]⁺. ¹H NMR |
| | (methanesulfonyl)ethyl]-1H- | (400 MHz, DMSO-d₆) δ 9.10 (s, 1H), 8.66 (s, |
| | pyrazol-4-yl}-8-phenyl-9H- | 1H), 8.34-8.27 (m, 2H), 7.75-7.71 (m, 2H), |
| | purine | 7.64-7.58 (m, 3H), 7.20 (t, J = 8.8 Hz, 2H), 4.80 |
| | | (t, J = 7.2 Hz, 2H), 3.88 (t, J = 7.2 Hz, 2H), 3.03 |
| | | (s, 3H) |
| 132 | tert-butyl 3-({4-[1-(1,1- | ¹H NMR (500 MHz, DMSO-d₆) δ 8.83 (s, 1H), |
| | dioxo-1λ6-thietan-3-yl)-3-(4- | 8.75 (s, 1H), 7.65-7.56 (m, 2H), 7.34 (s, 1H), |
| | fluorophenyl)-1H-pyrazol-4- | 7.24-7.15 (m, 2H), 5.47 (tt, J = 8.8, 5.5 Hz, |
| | yl]furo[2,3-d]pyrimidin-6- | 1H), 4.95-4.83 (m, 4H), 4.21 (t, J = 8.1 Hz, |
| | yl}ethynyl)azetidine-1- | 2H), 3.92-3.87 (m, 2H), 3.87-3.80 (m, 1H), |
| | carboxylate | 1.40 (s, 9H). m/z (ESI, +ve ion) = 564.4 [M + H]⁺ |
| 133 | 3-[4-{6-[(azetidin-3- | Formate salt: ¹H NMR (500 MHz, DMSO-d₆) δ |
| | yl)ethynyl]furo[2,3- | 8.83 (s, 1H), 8.75 (s, 1H), 8.21 (s, 1H), 7.67- |
| | d]pyrimidin-4-yl}-3-(4- | 7.55 (m, 2H), 7.29 (s, 1H), 7.25-7.14 (m, 2H), |
| | fluorophenyl)-1H-pyrazol-1- | 5.47 (tt, J = 8.7, 5.5 Hz, 1H), 4.98-4.82 (m, |
| | yl]-1λ6-thietane-1,1-dione | 4H), 3.90-3.82 (m, 1H), 3.79 (app t, J = 7.7 Hz, |
| | | 2H), 3.69 (app t, J = 7.3 Hz, 2H). m/z (ESI, +ve |
| | | ion) = 464.3 [M + H]⁺ |
| 139 | tert-butyl 5-{4-[1-(1,1-dioxo- | ¹H NMR (400 MHz, DMSO-d₆) δ 8.70 (s, 1H), |
| | 1λ6-thietan-3-yl)-3-(4- | 8.69 (s, 1H), 7.71 (br s, 1H), 7.63-7.53 (m, 2H), |
| | fluorophenyl)-1H-pyrazol-4- | 7.26-7.15 (m, 2H), 6.45 (s, 1H), 5.50 (dq, J = |
| | yl]furo[2,3-d]pyrimidin-6- | 8.4, 6.1 Hz, 1H), 4.94-4.81 (m, 4H), 3.60-3.55 |
| | yl}-3,4-dihydropyridine- | (m, 2H), 2.25 (app t, J = 5.9 Hz), 1.93-1.83 (m, |
| | 1(2H)-carboxylate | 2H), 1.50 (s, 9H). m/z (ESI, +ve ion) = 566.4 |
| | | [M + H]⁺ |
| 142 | 4-[1-(4,4- | ¹H NMR (400 MHz, DMSO-d₆) δ 8.77 (s, 1H), |
| | difluorocyclohexyl)-3-(4- | 8.64 (s, 1H), 7.96-7.91 (m, 2H), 7.63-7.54 (m, |
| | fluorophenyl)-1H-pyrazol-4- | 4H), 7.52-7.44 (m, 2H), 7.23-7.14 (m, 2H), |
| | yl]-6-phenylfuro[2,3- | 4.64-4.53 (m, 1H), 2.30-2.13 (m, 8H). m/z |
| | d]pyrimidine | (ESI, +ve ion) = 475.4 [M + H]⁺ |
| 145 | 4-[3-(4-fluorophenyl)-4-(8- | m/z (ESI, +ve ion) = 443.20 [M + H]⁺. ¹H NMR |
| | phenyl-9H-purin-6-yl)-1H- | (400 MHz, DMSO-d₆) δ 8.98 (s, 1H), 8.64 (s, |
| | pyrazol-1-yl]-2-methylbutan- | 1H), 8.32-8.25 (m, 2H), 7.76-7.68 (m, 2H), |
| | 2-ol | 7.64-7.56 (m, 3H), 7.18 (t, J = 8.8 Hz, 2H), 4.54 |
| | | (s, 1H), 4.44-4.36 (m, 2H), 2.11-2.02 (m, 2H), |
| | | 1.20 (s, 6H) |
| 146 | tert-butyl 5-{4-[1-(1,1-dioxo- | ¹H NMR (400 MHz, DMSO-d₆) δ 8.78 (s, 1H), |
| | 1λ6-thietan-3-yl)-3-(4- | 8.73 (s, 1H), 7.64-7.54 (m, 2H), 7.21 (t, J = 8.8 |
| | fluorophenyl)-1H-pyrazol-4- | Hz, 2H), 6.96-6.37 (m, 1H), 6.74 (s, 1H), 5.58- |
| | yl]furo[2,3-d]pyrimidin-6- | 5.43 (m, 1H), 4.97-4.80 (m, 4H), 4.13 (br s, |
| | yl}-3,6-dihydropyridine- | 2H), 3.50 (s, 2H), 2.34 (s, 2H), 1.44 (s, 9H). m/z |
| | 1(2H)-carboxylate | (ESI, +ve ion) = 566.3 [M + H]⁺ |
| 149 | 3-{4-[6-(4-acetylpiperazin-1- | ¹H NMR (400 MHz, DMSO-d₆) δ 8.56 (s, 1H), |
| | yl)furo[2,3-d]pyrimidin-4-yl]- | 8.49 (s, 1H), 7.58 (dd, J = 8.7, 5.6 Hz, 2H), 7.20 |
| | 3-(4-fluorophenyl)-1H- | (t, J = 8.9 Hz, 2H), 5.54-5.44 (m, 1H), 5.49 (s, |
| | pyrazol-1-yl}-1λ6-thietane- | 1H), 4.92-4.81 (m, 4H), 3.58 (s, 4H), 3.38- |
| | 1,1-dione | 3.33 (m, 2H), 3.30-3.25 (m, 2H), 2.05 (s, 3H). |
| | | m/z (ESI, +ve ion) = 511.3 [M + H]⁺ |
| 154 | 3-{3-(4-fluorophenyl)-4-[6- | ¹H NMR (400 MHz, DMSO-d₆) δ 8.60 (s, 1H), |
| | (1,4,5,6-tetrahydropyridin-3- | 8.50 (s, 1H), 7.61-7.54 (m, 2H), 7.24-7.16 (m, |
| | yl)furo[2,3-d]pyrimidin-4-yl]- | 3H), 6.55-6.50 (m, 1H), 5.76 (s, 1H), 5.54- |
| | 1H-pyrazol-1-yl}-1λ6- | 5.44 (m, 1H), 4.93-4.78 (m, 4H), 3.19-3.13 |
| | thietane-1,1-dione | (m, 2H), 2.15 (t, J = 6.1 Hz, 2H), 1.84-1.75 (m, |
| | | 2H). m/z (ESI, +ve ion) = 466.3 [M + H]⁺ |
| 159 | 3-{3-(4-fluorophenyl)-4-[6- | ¹H NMR (400 MHz, DMSO-d₆) δ 8.76 (s, 1H), |
| | (1,2,5,6-tetrahydropyridin-3- | 8.70 (s, 1H), 7.59 (dd, J = 8.8, 5.6 Hz, 2H), 7.21 |
| | yl)furo[2,3-d]pyrimidin-4-yl]- | (t, J = 8.9 Hz, 2H), 6.67 (s, 1H), 6.50 (s, 1H), |
| | 1H-pyrazol-1-yl}-1λ6- | 5.55-5.44 (m, 1H), 4.97-4.77 (m, 4H), 3.40 (s, |
| | thietane-1,1-dione | 2H), 2.83 (t, J = 5.4 Hz, 2H), 2.27-2.17 (m, |
| | | 2H). m/z (ESI, +ve ion) = 466.3 [M + H]⁺ |
| 160 | 3-{3-(4-fluorophenyl)-4-[6- | ¹H NMR (400 MHz, DMSO-d₆) δ 8.74 (s, 1H), |
| | (2-oxopiperidin-1- | 8.63 (s, 1H), 7.57 (dd, J = 8.7, 5.6 Hz, 2H), 7.19 |
| | yl)furo[2,3-d]pyrimidin-4-yl]- | (t, J = 8.9 Hz, 2H), 6.69 (s, 1H), 5.58-5.48 (m, |
| | 1H-pyrazol-1-yl}-1λ6- | 1H), 4.95-4.78 (m, 4H), 3.88 (t, J = 6.0 Hz, |
| | thietane-1,1-dione | 2H), 2.55-2.51 (m, 2H), 1.95-1.86 (m, 2H), |
| | | 1.86-1.76 (m, 2H). m/z (ESI, +ve ion) = 482.3 |
| | | M + H]⁺ |
| 168 | 3-{3-(4-fluorophenyl)-4-[6- | ¹H NMR (400 MHz, DMSO-d₆) δ 8.56 (s, 1H), |
| | (6-oxa-2-azaspiro[3.4]octan- | 8.48 (s, 1H), 7.57 (dd, J = 8.8, 5.6 Hz, 2H), 7.20 |
| | 2-yl)furo[2,3-d]pyrimidin-4- | (t, J = 8.9 Hz, 2H), 5.53-5.44 (m, 1H), 5.29 (s, |
| | yl]-1H-pyrazol-1-yl}-1λ6- | 1H), 4.93-4.80 (m, 4H), 4.00 (s, 4H), 3.82 (s, |
| | thietane-1,1-dione | 2H), 3.72 (t, J = 7.0 Hz, 2H), 2.17 (t, J = 7.0 Hz, |
| | | 2H). m/z (ESI, +ve ion) = 496.3 [M + H]+ |
| 169 | 3-[3-(4-fluorophenyl)-4-{6- | ¹H NMR (400 MHz, DMSO-d₆) δ 8.85 (s, 1H), |
| | [3- | 8.81 (s, 1H), 8.24-8.18 (m, 2H), 7.89-7.79 (m, |
| | (trifluoromethyl)phenyl]furo[ | 2H), 7.66 (s, 1H), 7.68-7.62 (m, 2H), 7.25- |
| | 2,3-d]pyrimidin-4-yl}-1H- | 7.16 (m, 2H), 5.57-5.49 (m, 1H), 4.98-4.84 |
| | pyrazol-1-yl]-1λ6-thietane- | (m, 4H). m/z (ESI, +ve ion) = 529.2 [M + H]+ |
| | 1,1-dione | |
| 170 | 4-[1-(3,3-difluorocyclobutyl)- | m/z (ESI, +ve ion) = 447.00 [M + H]⁺. ¹H NMR |
| | 3-(4-fluorophenyl)-1H- | (400 MHz, Chloroform-d) δ 8.93 (s, 1H), 8.15 (s, |
| | pyrazol-4-yl]-6- | 1H), 7.68-7.61 (m, 2H), 7.65-7.52 (m, 2H), |
| | phenylfuro[2,3-d]pyrimidine | 7.49-7.37 (m, 3H), 7.17-7.05 (m, 2H), 6.09 (s, |
| | | 1H), 4.85 (m, 1H), 3.50-3.40 (m, 2H), 3.23- |
| | | 3.17 (m, 2H) |
| 172 | 4-{3-(4-fluorophenyl)-1- | m/z (ESI, +ve ion) = 467.10 [M + H]⁺. ¹H-NMR |
| | [(1R,3r,5S)-8- | (400 MHz, Chloroform-d) δ 8.93 (s, 1H), 8.21 (s, |
| | oxabicyclo[3.2.1]octan-3-yl]- | 1H), 7.65-7.63 (m, 2H), 7.58-7.55 (m, 2H), |
| | 1H-pyrazol-4-yl}-6- | 7.46-7.40 (m, 3H), 7.12-7.07 (m, 2H), 6.09 (s, |
| | phenylfuro[2,3-d]pyrimidine | 1H), 4.62-4.56 (m, 1H), 4.53 (s, 2H), 2.63-2.50 |
| | | (m, 4H), 2.04-1.90 (m, 2H), 1.88-1.82 (m, 2H) |
| 174 | 3-{3-(4-fluorophenyl)-4-[6- | m/z (ESI, +ve ion) = 463.10 [M + H]⁺. ¹H NMR |
| | (pyridazin-4-yl)furo[2,3- | (400 MHz, Chloroform-d) δ 9.74 (s, 1H), 9.48- |
| | d]pyrimidin-4-yl]-1H- | 9.39 (m, 1H), 8.91 (s, 1H), 8.80 (s, 1H), 8.14- |
| | pyrazol-1-yl}-1λ6-thietane- | 8.06 (m, 1H), 7.99 (s, 1H), 7.75-7.55 (m, 2H), |
| | 1,1-dione | 7.28-7.14 (m, 2H), 5.74-5.31 (m, 1H), 5.20- |
| | | 4.62 (m, 4H). |
| 175 | 3-{3-(4-fluorophenyl)-4-[6- | m/z (ESI, +ve ion) = 463.15 [M + H]⁺. ¹H NMR |
| | (pyrimidin-5-yl)furo[2,3- | (400 MHz, DMSO-d₆) δ 9.32 (s, 2H), 9.28 (s, |
| | d]pyrimidin-4-yl]-1H- | 1H) 8.88 (s, 1H), 8.77 (s, 1H), 7.75 (s, 1H), 7.68- |
| | pyrazol-1-yl}-1λ6-thietane- | 7.58 (m, 2H), 7.33-7.08 (m, 2H), 5.54 (s, 1H), |
| | 1,1-dione | 4.99-4.73 (m, 4H) |
| 176 | 3-{4-[1-(1,1-dioxo-1λ6- | ¹H NMR (400 MHz, DMSO-d₆) δ 8.82 (s, 2H), |
| | thietan-3-yl)-3-(4- | 8.44 (s, 1H), 8.19 (s, 1H), 8.05 (d, J = 7.8 Hz, |
| | fluorophenyl)-1H-pyrazol-4- | 1H), 7.98 (d, J = 7.8 Hz, 1H), 7.70-7.60 (m, |
| | yl]furo[2,3-d]pyrimidin-6- | 3H), 7.60-7.52 (m, 1H), 7.56 (s, 1H), 7.27- |
| | yl}benzamide | 7.16 (m, 2H), 5.58-5.48 (m, 1H), 4.98-4.83 |
| | | (m, 4H). m/z (ESI, +ve ion) = 504.3 [M + H]⁺ |
| 180 | 3-{4-[6-(1-benzoyl-1,2,3,6- | ¹H NMR (400 MHz, DMSO-d₆) δ 8.78 (s, 1H), |
| | tetrahydropyridin-4- | 8.71 (s, 1H), 7.59 (dd, J = 8.7, 5.6 Hz, 2H), 7.54- |
| | yl)furo[2,3-d]pyrimidin-4-yl]- | 7.40 (m, 6H), 7.22 (t, J = 8.9 Hz, 2H), 6.70 (s, |
| | 3-(4-fluorophenyl)-1H- | 1H), 5.56-5.45 (m, 1H), 4.94-4.82 (m, 4H), |
| | pyrazol-1-yl}-1λ6-thietane- | 4.36 (s, 1H), 4.17 (s, 1H), 3.86 (s, 1H), 3.54 (s, |
| | 1,1-dione | 1H), 2.44 (s, 2H). m/z (ESI, +ve ion) = 570.4 |
| | | [M + H]⁺ |
| 182 | 3-[3-(4-fluorophenyl)-4-{6- | ¹H NMR (400 MHz, DMSO-d₆) δ 8.89 (s, 1H), |
| | [2- | 8.77 (s, 1H), 7.97 (d, J = 7.9 Hz, 1H), 7.90-7.84 |
| | (trifluoromethyl)phenyl]furo[2,3- | (m, 2H), 7.82-7.74 (m, 1H), 7.66-7.59 (m, |
| | d]pyrimidin-4-yl}-1H- | 2H), 7.21 (app t, J = 8.8 Hz, 2H), 7.08 (s, 1H), |
| | pyrazol-1-yl]-1λ6-thietane- | 5.58-5.38 (m, 1H), 4.96-4.74 (m, 4H). m/z |
| | 1,1-dione | (ESI, +ve ion) = 529.2 [M + H]⁺ |
| 183 | 2-{4-[1-(1,1-dioxo-1λ6- | ¹H NMR (400 MHz, DMSO-d₆) δ 8.82 (s, 1H), |
| | thietan-3-yl)-3-(4- | 8.73 (s, 1H), 7.95 (br s, 1H), 7.78-7.72 (m, 1H), |
| | fluorophenyl)-1H-pyrazol-4- | 7.64-7.50 (m, 6H), 7.21 (app t, J = 8.9 Hz, 2H), |
| | yl]furo[2,3-d]pyrimidin-6- | 7.08 (s, 1H), 5.59-5.46 (m, 1H), 4.96-4.83 (m, |
| | yl}benzamide | 4H). m/z (ESI, +ve ion) = 504.3 [M + H]⁺ |
| 185 | 4-{3-(4-fluorophenyl)-1-[3- | m/z (ESI, +ve ion) = 503.05 [M + H]⁺. ¹H NMR |
| | (methanesulfonyl)cyclopentyl]- | (400 MHz, Chloroform-d) δ 8.91 (s, 1H), 8.09 (s, |
| | 1H-pyrazol-4-yl}-6- | 1H), 7.71-7.62 (m, 2H), 7.58-7.50 (m, 2H), |
| | phenylfuro[2,3-d]pyrimidine | 7.49-7.36 (m, 3H), 7.09 (t, J = 8.4 Hz, 2H), 6.14 |
| | | (s, 1H), 5.17-4.93 (m, 1H), 3.94-3.92 (m, 1H), |
| | | 2.96 (s, 3H), 2.77-12.75 (m, 2H), 2.57-2.21 |
| | | (m, 4H) |
| 186 | 4-{3-(4-fluorophenyl)-1- | m/z (ESI, +ve ion) = 467.15 [M + H]⁺. ¹H-NMR |
| | [(1R,3s,5S)-8- | (400 MHz, Chloroform-d) δ 8.92 (s, 1H), 8.12 (s, |
| | oxabicyclo[3.2.1]octan-3-yl]- | 1H), 7.80-7.34 (m, 7H), 7.11-7.06 (m, 2H), |
| | 1H-pyrazol-4-yl}-6- | 6.12 (s, 1H), 4.79-4.70 (m, 1H), 4.67-4.60 (m, |
| | phenylfuro[2,3-d]pyrimidine | 2H), 2.23-2.27 (m, 2H), 2.21-2.11 (m, 4H), |
| | | 1.94-1.92 (m, 2H) |
| 187 | 3-{3-(4-fluorophenyl)-4-[6- | m/z (ESI, +ve ion) = 463.05 [M + H]⁺. ¹H NMR |
| | (pyridazin-3-yl)furo[2,3- | (400 MHz, DMSO-d₆) δ 9.74 (s, 1H), 9.41 (s, |
| | d]pyrimidin-4-yl]-1H- | 1H), 8.91 (s, 1H), 8.80 (s, 1H), 8.11-8.09 (m, |
| | pyrazol-1-yl}-1λ6-thietane- | 1H), 7.99 (s, 1H), 7.67-7.64 (m, 2H), 7.22 (t, J = |
| | 1,1-dione | 9.2 Hz, 2H), 5.55-5.50 (m, 1H), 4.93 (d, J = 7.2 |
| | | Hz, 4H) |
| 188 | 3-{3-(4-fluorophenyl)-4-[6- | m/z (ESI, +ve ion) = 463.05 [M + H]⁺. ¹H NMR |
| | (pyrazin-2-yl)furo[2,3- | (400 MHz, DMSO-d₆) 9.24 (d, J = 1.6 Hz, 1H), |
| | d]pyrimidin-4-yl]-1H- | 8.89-8.82 (m, 2H), 8.81 (s, 1H), 8.81-8.75 (m, |
| | pyrazol-1-yl}-1λ6-thietane- | 1H), 7.76 (s, 1H), 7.72-7.57 (m, 2H), 7.22 (t, J = |
| | 1,1-dione | 8.8 Hz, 2H), 5.56-5.51 (m, 1H), 4.94-4.89 (m, 4H) |
| 191 | 3-{4-[8-(1-acetyl-1,2,3,6- | ¹H NMR(400 MHz, DMSO-d₆) δ 9.03 (s, 1H), |
| | tetrahydropyridin-4-yl)-9H- | 8.61 (s, 1H), 7.73-7.69 (m, 2H), 7.22-7.17 (m, |
| | purin-6-yl]-3-(4- | 2H), 6.93 (s, 1H), 5.66-5.61 (m, 1H), 4.86- |
| | fluorophenyl)-1H-pyrazol-1- | 4.84 (m, 4H), 4.28 (app s, 1H), 4.21 (app s, 1H), |
| | yl}-1λ6-thietane-1,1-dione | 3.68-3.63 (m, 2H), 2.73 (app s, 1H), 2.63 (app |
| | | s, 1H), 2.10 and 2.07 (s, 3H). Note: coalescence |
| | | observed for peaks at 2.10 and 2.07 ppm at 90° C. |
| | | m/z (ESI, +ve ion) = 508.3 [M + H]⁺ |
| 192 | 4-{3-(4-fluorophenyl)-1- | m/z (ESI, +ve ion) = 503.15 [M + H]⁺. ¹H NMR |
| | [(1S,3R)-3- | (400 MHz, Chloroform-d) δ 8.96 (s, 1H), 8.31 (s, |
| | (methanesulfonyl)cyclopentyl]- | 1H), 7.70-7.60 (m, 2H), 7.59-7.51 (m, 2H), |
| | 1H-pyrazol-4-yl}-6- | 7.50-7.41 (m, 3H), 7.12 (t, J = 8.8 Hz, 2H), 6.08 |
| | phenylfuro[2,3-d]pyrimidine | (s, 1H), 5.22-4.82 (m, 1H), 4.05-3.76 (m, 1H), |
| | (absolute stereochemistry | 2.98 (s, 3H), 2.76-2.74 (m, 2H), 2.61-2.18 (m, |
| | arbitrarily assigned) | 4H) |
| 193 | 4-{3-(4-fluorophenyl)-1- | m/z (ESI, +ve ion) = 503.15 [M + H]⁺. ¹H NMR |
| | [(1R,3S)-3- | (400 MHz, Chloroform-d) δ 9.00 (s, 1H), 8.49 (s, |
| | (methanesulfonyl)cyclopentyl]- | 1H), 7.63-7.62 (m, 2H), 7.54-7.53 (m, 2H), |
| | 1H-pyrazol-4-yl}-6- | 7.50-7.40 (m, 3H), 7.13 (t, J = 8.4 Hz, 2H), 6.03 |
| | phenylfuro[2,3-d]pyrimidine | (s, 1H), 5.14-4.82 (m, 1H), 4.01-3.98 (m, 1H), |
| | (absolute stereochemistry | 2.98 (s, 3H), 2.77-2.74 (m, 2H), 2.55-2.38 (m, |
| | arbitrarily assigned) | 4H) |
| 194 | 4-{3-(4-fluorophenyl)-1- | m/z (ESI, +ve ion) = 489.10 [M + H]⁺. ¹H NMR |
| | [(1r,3r)-3- | (400 MHz, Chloroform-d) δ 8.91 (s, 1H), 8.07 (s, |
| | (methanesulfonyl)cyclobutyl]- | 1H), 7.68-7.65 (m, 2H), 7.60-7.56(m, 2H), |
| | 1H-pyrazol-4-yl}-6- | 7.48-7.39 (m, 3H), 7.14-7.08 (m, 2H), 6.15 (s, |
| | phenylfuro[2,3-d]pyrimidine | 1H), 5.27-5.18 (m, 1H), 3.99-3.92 (m, 1H), |
| | | 3.34-3.26 (m, 2H), 3.17-3.10 (m, 2H), 2.97 (s, 3H) |
| 195 | 3-{3-(4-fluorophenyl)-4-[6- | m/z (ESI, +ve ion) = 463.00 [M + H]⁺. ¹H NMR |
| | (pyrimidin-2-yl)furo[2,3- | (400 MHz, Chloroform-d) δ 9.04 (s, 1H), 8.85- |
| | d]pyrimidin-4-yl]-1H- | 8.84 (d, J = 4.8 Hz, 2H), 8.26 (s, 1H), 7.59-7.54 |
| | pyrazol-1-yl}-1λ6-thietane- | (m, 2H), 7.31-7.28 (m, 1H), 7.11-7.06 (m, 2H), |
| | 1,1-dione | 7.01 (s, 1H), 5.34-5.28 (m, 1H), 5.00-4.94 (m, |
| | | 2H), 4.76-4.70 (m, 2H) |
| 196 | 3-{3-(4-fluorophenyl)-4-[6- | m/z (ESI, +ve ion) = 463.00 [M + H]⁺. ¹H NMR |
| | (pyrimidin-4-yl)furo[2,3- | (400 MHz, Chloroform-d) 9.24 (s, 1H), 9.02 (s, |
| | d]pyrimidin-4-yl]-1H- | 1H), 8.91 (s, 1H), 8.25 (s, 1H), 7.85-7.83 (d, J = |
| | pyrazol-1-yl}-1λ6-thietane- | 5.2 Hz, 1H), 7.59-7.54 (m, 2H), 7.12-7.01 (m, |
| | 1,1-dione | 2H), 7.00 (s, 1H), 5.37-5.28 (m, 1H), 4.99-4.94 |
| | | (m, 2H), 4.76-4.70 (m, 2H) |
| 199 | 2,5-anhydro-1,3,4-trideoxy-3- | m/z (ESI, +ve ion) = 441.15 [M + H]⁺. ¹H NMR |
| | [3-(4-fluorophenyl)-4-(6- | (400 MHz, Chloroform-d) δ 8.92 (s, 1H), 8.13 (s, |
| | phenylfuro[2,3-d]pyrimidin- | 1H), 7.66-7.62 (m, 2H), 7.59-7.53 (m, 2H), |
| | 4-yl)-1H-pyrazol-1- | 7.47-7.38 (m, 3H), 7.10 (t, J = 8.6 Hz, 2H), 6.09 |
| | yl]pentitol | (s, 1H), 4.53-4.50 (m, 1H), 4.38-4.32 (m, 1H), |
| | | 4.24-4.16 (m, 2H), 2.68-2.56 (m, 2H), 1.43 (d, |
| | | J = 6.4 Hz, 3H). |
| 201 | 3-{3-(4-fluorophenyl)-4-[6- | m/z (ESI, +ve ion) = 465.10 [M + H]⁺. ¹H NMR |
| | (1-methyl-1H-imidazol-2- | (400 MHz, DMSO-d₆) δ 8.84 (s, 1H), 8.75 (s, |
| | yl)furo[2,3-d]pyrimidin-4-yl]- | 1H), 7.89 (s, 1H), 7.65-7.57 (m, 2H), 7.53 (d, J = |
| | 1H-pyrazol-1-yl}-1λ6- | 2.0 Hz, 1H), 7.28-7.19 (m, 2H), 6.77 (s, 1H), |
| | thietane-1,1-dione | 5.59-5.47 (m, 1H), 4.90 (d, J = 8.0 Hz, 4H), |
| | | 3.75 (s, 3H) |
| 202 | 4-[1-(2,2-dimethyloxan-4-yl)- | m/z (ESI, +ve ion) = 469.20 [M + H]⁺. ¹H NMR |
| | 3-(4-fluorophenyl)-1H- | (400 MHz, Chloroform-d) δ 8.92 (s, 1H), 8.12 (s, |
| | pyrazol-4-yl]-6- | 1H), 7.61 (d, J = 7.2 Hz, 2H), 7.57-7.51 (m, |
| | phenylfuro[2,3-d]pyrimidine | 2H), 7.47-7.38 (m, 3H), 7.13 (t, J = 8.4 Hz, 2H), |
| | | 5.98 (s, 1H), 4.74-4.64 (m, 1H), 4.04-3.97 (m, |
| | | 1H), 3.87 (t, J = 12.0 Hz, 1H), 2.28-2.20 (m, |
| | | 2H), 2.19-1.98 (m, 2H), 1.37 (s, 6H) |
| 204 | 4-{1-[(4,4- | ¹H NMR (400 MHz, DMSO-d₆) δ 8.77 (s, 1H), |
| | difluorocyclohexyl)methyl]- | 8.65 (s, 1H), 7.91 (dd, J = 5.2, 3.3 Hz, 2H), 7.63- |
| | 3-(4-fluorophenyl)-1H- | 7.53 (m, 4H), 7.53-7.46 (m, 1H), 7.36 (s, 1H), |
| | pyrazol-4-yl}-6- | 7.23-7.14 (m, 2H), 4.21 (d, J = 7.2 Hz, 2H), |
| | phenylfuro[2,3-d]pyrimidine | 2.22-2.11 (m, 1H), 2.11-1.98 (m, 2H), 1.95- |
| | | 1.65 (m, 4H), 1.42-1.28 (m, 2H). m/z (ESI, +ve |
| | | ion) = 489.5 [M + H]⁺ |
| 206 | 3-{4-[6- | ¹H NMR (400 MHz, DMSO-d₆) δ 8.81 (s, 1H), |
| | (cyclobutylethynyl)furo[2,3- | 8.75 (s, 1H), 7.62 (dd, J = 8.8, 5.6 Hz, 2H), 7.28 |
| | d]pyrimidin-4-yl]-3-(4- | (s, 1H), 7.21 (app t, J = 8.9 Hz, 2H), 5.53-5.40 |
| | fluorophenyl)-1H-pyrazol-1- | (m, 1H), 4.98-4.78 (m, 4H), 3.53-3.38 (m, |
| | yl}-1λ6-thietane-1,1-dione | 1H), 2.41-2.27 (m, 2H), 2.27-2.11 (m, 2H), |
| | | 2.09-1.83 (m, 2H). m/z (ESI, +ve ion) = 463.2 |
| | | [M + H]⁺ |
| 207 | 4-[1-(2,2- | ¹H NMR (400 MHz, DMSO-d₆) δ 8.80 (s, 1H), |
| | difluorocyclohexyl)-3-(4- | 8.61 (s, 1H), 7.93-7.87 (m, 2H), 7.61-7.53 (m, |
| | fluorophenyl)-1H-pyrazol-4- | 4H), 7.52-7.47 (m, 1H), 7.25 (s, 1H), 7.23- |
| | yl]-6-phenylfuro[2,3- | 7.15 (m, 2H), 5.02-4.88 (m, 1H), 2.47-2.35 |
| | d]pyrimidine | (m, 1H), 2.30-2.15 (m, 2H), 2.12-1.78 (m, |
| | | 3H), 1.67-1.48 (m, 2H). m/z (ESI, +ve ion) = |
| | | 475.8 [M + H]⁺ |
| 209 | 3-{4-[6-(1-acetyl-1,2,5,6- | ¹H NMR (400 MHz, DMSO-d₆, 90° C.) δ 8.71 (s, |
| | tetrahydropyridin-3- | 1H), 8.38 (s, 1H), 7.60-7.52 (m, 2H), 7.17- |
| | yl)furo[2,3-d]pyrimidin-4-yl]- | 7.07 (m, 2H), 6.68 (s, 1H), 6.54 (s, 1H), 4.55 (s, |
| | 3-(4-fluorophenyl)-1H- | 1H), 4.25-4.13 (m, 4H), 3.57 (t, J = 5.4 Hz, |
| | pyrazol-1-yl}-1λ6-thietane- | 2H), 2.37 (s, 2H), 2.06 (s, 3H), 1.18 (s, 6H). m/z |
| | 1,1-dione | (ESI, +ve ion) = 476.4 [M + H]⁺ |
| 213 | 4-(3-(4-fluorophenyl)-1- | m/z (ESI, +ve ion) = 441.10 [M + H]⁺. ¹H NMR |
| | ((2S,3R)-2- | (400 MHz, Chloroform-d) δ 8.94 (s, 1H), 8.20 (s, |
| | methyltetrahydrofuran-3-yl)- | 1H), 7.65-7.62 (m, 2H), 7.59-7.54 (m, 2H), |
| | 1H-pyrazol-4-yl)-6- | 7.48-7.40 (m, 3H), 7.11 (t, J = 8.6 Hz, 2H), 6.08 |
| | phenylfuro[2,3-d]pyrimidine | (s, 1H), 4.56-4.51 (m, 1H), 4.38-4.32 (m, 1H), |
| | (absolute stereochemistry | 4.24-4.15 (m, 2H), 2.68-2.57 (m, 2H), 1.43 (d, |
| | arbitrarily assigned) | J = 6.0 Hz, 3H) |
| 214 | 4-(3-(4-fluorophenyl)-1- | m/z (ESI, +ve ion) = 441.05 [M + H]⁺. ¹H NMR |
| | ((2R,3S)-2- | (400 MHz, Chloroform-d) δ 8.95 (s, 1H), 8.25 (s, |
| | methyltetrahydrofuran-3-yl)- | 1H), 7.65-7.63 (m, 2H), 7.58-7.54 (m, 2H), |
| | 1H-pyrazol-4-yl)-6- | 7.48-7.40 (m, 3H), 7.11 (t, J = 8.6 Hz, 2H), 6.07 |
| | phenylfuro[2,3-d]pyrimidine | (s, 1H), 4.56-4.53 (m, 1H), 4.38-4.32 (m, 1H), |
| | (absolute stereochemistry | 4.24-4.15 (m, 2H), 2.68-2.57 (m, 2H), 1.43 (d, |
| | arbitrarily assigned) | J = 6.0 Hz, 3H) |
| 218 | 3-{3-(4-fluorophenyl)-4-[6- | m/z (ESI, +ve ion) = 467.95 [M + H]⁺. ¹H NMR |
| | (1,2-thiazol-5-yl)furo[2,3- | (400 MHz, Chloroform-d) δ 9.01 (s, 1H), 8.52- |
| | d]pyrimidin-4-yl]-1H- | 8.51 (d, J = 1.7 Hz, 1H), 8.37 (s, 1H), 7.56-7.52 |
| | pyrazol-1-yl}-1λ6-thietane- | (m, 2H), 7.46-7.45 (d, J = 1.8 Hz, 1H), 7.16- |
| | 1,1-dione | 7.12 (m, 2H), 6.04 (s, 1H), 5.33 (s, 1H), 4.97- |
| | | 4.94 (m, 2H), 4.77-4.75 (m, 2H) |
| 219 | 4-{1-[(1r,3r)-3- | m/z (ESI, +ve ion) = 429.05 [M + H]⁺. ¹H NMR |
| | fluorocyclobutyl]-3-(4- | (400 MHz, DMSO-d₆) δ 8.78 (s, 1H), 8.70 (s, |
| | fluorophenyl)-1H-pyrazol-4- | 1H), 7.94-7.92 (d, J = 7.6 Hz, 2H), 7.65-7.45 |
| | yl}-6-phenylfuro[2,3- | (m, 6H), 7.23-7.20 (m, 2H), 5.60-5.46 (m, 1H), |
| | d]pyrimidine | 5.28 (s, 1H), 3.04-2.95 (m, 2H), 2.90-2.82 (m, 2H) |
| 223 | 3-{3-(4-fluorophenyl)-4-[6- | m/z (ESI, +ve ion) = 467.00 [M + H]⁺. ¹H NMR |
| | (3-methyl-1,2,4-oxadiazol-5- | (400 MHz, Chloroform-d) δ 9.10 (s, 1H), 8.28 (s, |
| | yl)furo[2,3-d]pyrimidin-4-yl]- | 1H), 7.55-7.51 (m, 2H), 7.13-7.09 (m, 2H), |
| | 1H-pyrazol-1-yl}-1λ6- | 6.77 (s, 1H), 5.36-5.28 (m, 1H), 4.98-4.92 (m, |
| | thietane-1,1-dione | 2H), 4.77-4.71 (m, 2H), 2.52 (s, 3H) |
| 225 | 4-{1-[(4R)-2,2-dimethyloxan- | m/z (ESI, +ve ion) = 469.30 [M + H]⁺. ¹H NMR |
| | 4-yl]-3-(4-fluorophenyl)-1H- | (400 MHz, Chloroform-d) δ 8.92 (s, 1H), 8.12 (s, |
| | pyrazol-4-yl}-6- | 1H), 7.63 (d, J = 7.2 Hz, 2H), 7.57-7.53 (m, |
| | phenylfuro[2,3-d]pyrimidine | 2H), 7.47-7.38 (m, 3H), 7.10 (t, J = 8.4 Hz, 2H), |
| | (absolute stereochemistry | 6.05 (s, 1H), 4.74-4.63 (m, 1H), 4.02-3.96 (m, |
| | arbitrarily assigned) | 1H), 3.87 (t, J = 12.0 Hz, 1H), 2.28-2.18 (m, |
| | | 2H), 2.16-1.94 (m, 2H), 1.37 (d, J = 4.0 Hz, 6H) |
| 226 | 4-{1-[(4S)-2,2-dimethyloxan- | m/z (ESI, +ve ion) = 469.30 [M + H]⁺. ¹H NMR |
| | 4-yl]-3-(4-fluorophenyl)-1H- | (400 MHz, Chloroform-d) δ 9.04 (s, 1H), 8.71 (s, |
| | pyrazol-4-yl}-6- | 1H), 7.62-7.58 (m, 2H), 7.57-7.52 (m, 2H), |
| | phenylfuro[2,3-d]pyrimidine | 7.51-7.46 (m, 3H), 7.29-7.02 (m, 2H), 5.94 (s, |
| | (absolute stereochemistry | 1H), 4.74-4.67 (m, 1H), 4.02-3.97 (m, 1H), |
| | arbitrarily assigned) | 3.91-3.84 (m, 1H), 2.28-2.18 (m, 2H), 2.16- |
| | | 2.10 (m, 1H), 2.10-1.99 (m, 1H), 1.37 (d, J = |
| | | 4.0 Hz, 6H) |
| 228 | 2-{4-[1-(1,1-dioxo-1λ6- | ¹H NMR (400 MHz, DMSO-d₆) δ 8.84 (s, 1H), |
| | thietan-3-yl)-3-(4- | 8.72 (s, 1H), 7.93-7.86 (m, 1H), 7.68-7.51 (m, |
| | fluorophenyl)-1H-pyrazol-4- | 4H), 7.40-7.35 (m, 1H), 7.25-7.15 (m, 2H), |
| | yl]furo[2,3-d]pyrimidin-6- | 7.04 (s, 1H), 5.58-5.45 (m, 1H), 4.99-4.82 (m, |
| | yl}-N,N-dimethylbenzamide | 4H), 3.00 (s, 3H), 2.70 (s, 3H). m/z (ESI, +ve |
| | | ion) = 532.2 [M + H]⁺ |
| 229 | 2-{4-[1-(1,1-dioxo-1λ6- | ¹H NMR (400 MHz, DMSO-d₆) δ 8.82 (s, 1H), |
| | thietan-3-yl)-3-(4- | 8.72 (s, 1H), 8.40 (d, J = 3.9 Hz, 1H), 7.79 (d, J = |
| | fluorophenyl)-1H-pyrazol-4- | 7.7 Hz, 1H), 7.66-7.45 (m, 5H), 7.21 (app t, J = |
| | yl]furo[2,3-d]pyrimidin-6- | 8.8 Hz, 2H), 7.04 (s, 1H), 5.57-5.47 (m, 1H), |
| | yl}-N-methylbenzamide | 4.97-4.82 (m, 4H), 2.73 (d, J = 4.6 Hz, 3H). |
| | | m/z (ESI, +ve ion) = 518.2 [M + H]⁺ |
| 230 | 4-{1-[(1R)-2,2- | ¹H NMR (400 MHz, DMSO-d₆) δ 8.80 (s, 1H), |
| | difluorocyclohexyl]-3-(4- | 8.61 (s, 1H), 7.93-7.88 (m, 2H), 7.61-7.53 (m, |
| | fluorophenyl)-1H-pyrazol-4- | 4H), 7.53-7.46 (m, 1H), 7.25 (s, 1H), 7.20 (t, J = |
| | yl}-6-phenylfuro[2,3- | 8.9 Hz, 2H), 4.94 (dt, J = 27.3, 13.8 Hz, 1H), |
| | d]pyrimidine | 2.46-2.35 (m, 1H), 2.30-2.14 (m, 2H), 2.12- |
| | (absolute stereochemistry | 1.79 (m, 3H), 1.67-1.52 (m, 2H). m/z (ESI, +ve |
| | arbitrarily assigned) | ion) = 475.4 [M + H]⁺ |
| 233 | 4-{1-[(1S)-2,2- | ¹H NMR (400 MHz, DMSO-d₆) δ 8.80 (s, 1H), |
| | difluorocyclohexyl]-3-(4- | 8.61 (s, 1H), 7.94-7.88 (m, 2H), 7.58 (ddd, J = |
| | fluorophenyl)-1H-pyrazol-4- | 14.5, 6.8, 5.4 Hz, 4H), 7.53-7.46 (m, 1H), 7.25 |
| | yl}-6-phenylfuro[2,3- | (s, 1H), 7.24-7.15 (m, 2H), 5.02-4.89 (m, 1H), |
| | d]pyrimidine | 2.45-2.36 (m, 1H), 2.33-2.15 (m, 2H), 2.13- |
| | (absolute stereochemistry | 1.80 (m, 3H), 1.65-1.52 (m, 2H). m/z (ESI, +ve |
| | arbitrarily assigned) | ion) = 475.4 [M + H]⁺ |
| 234 | 3-{3-(4-fluorophenyl)-4-[6- | m/z (ESI, +ve ion) = 466.20 [M + H]⁺. ¹H NMR |
| | (5-methyl-1,3-oxazol-2- | (400 MHz, Chloroform-d) δ 9.04 (s, 1H), 8.42 (s, |
| | yl)furo[2,3-d]pyrimidin-4-yl]- | 1H), 7.54 (s, 2H), 7.11 (s, 2H), 6.97 (s, 1H), 6.45 |
| | 1H-pyrazol-1-yl}-1λ6- | (s, 1H), 5.33 (s, 1H), 4.96 (d, J = 12.0 Hz, 2H), |
| | thietane-1,1-dione | 4.74 (s, 2H), 2.44 (s, 3H) |
| 239 | 5-{4-[1-(1, 1-dioxo-1λ6- | m/z (ESI, +ve ion) = 519.05 [M + H]⁺. ¹H NMR |
| | thietan-3-yl)-3-(4- | (400 MHz, DMSO-d₆) δ 9.25 (d, J = 2.4 Hz, 1H), |
| | fluorophenyl)-1H-pyrazol-4- | 9.07 (d, J = 2.0 Hz, 1H), 8.86 (s, 2H), 8.81 (s, |
| | yl]furo[2,3-d]pyrimidin-6- | 1H), 8.69 (s, 1H), 7.76 (s, 1H), 7.70-7.62 (m, |
| | yl}-N-methylpyridine-3- | 2H), 7.22 (t, J = 8.8 Hz, 2H), 5.60-5.48 (m, 1H), |
| | carboxamide | 4.98-4.86 (m, 4H), 2.87 (d, J = 4.4 Hz, 3H) |
| 241 | 3-{3-(4-fluorophenyl)-4-[6- | m/z (ESI, +ve ion) = 475.95 [M + H]⁺. ¹H-NMR |
| | (3-methylpyridin-2- | (400 MHz, Chloroform-d) δ 9.01 (s, 1H), 8.56- |
| | yl)furo[2,3-d]pyrimidin-4-yl]- | 8.55 (d, J = 4 Hz, 1H), 8.26 (s, 1H), 7.66-7.58 |
| | 1H-pyrazol-1-yl}-1λ6- | (m, 3H), 7.30 (s, 1H), 7.10-7.05 (m, 2H), 6.85 |
| | thietane-1,1-dione | (s, 1H), 5.34-5.26 (m, 1H), 5.00-4.94 (m, 2H), |
| | | 4.75-4.69 (m, 2H), 2.56 (s, 3H) |
| 249 | 3-{3-(4-fluorophenyl)-4-[6- | m/z (ESI, +ve ion) = 476.00 [M + H]⁺. ¹H-NMR |
| | (2-methylpyridin-3- | (400 MHz, DMSO-d₆) δ 8.90 (s, 1H), 8.77 (s, |
| | yl)furo[2,3-d]pyrimidin-4-yl]- | 1H), 8.56-8.55 (d, J = 4 Hz, 1H), 8.17 (t, J = 4 |
| | 1H-pyrazol-1-yl}-1λ6- | Hz, 1H), 7.64-7.61 (m, 2H), 7.46-7.42 (m, |
| | thietane-1,1-dione | 1H), 7.24 (t, J = 16 Hz, 2H), 6.98 (s, 1H), 5.55- |
| | | 5.50 (m, 1H), 4.93-4.89 (t, J = 16 Hz, 4H), 2.58 |
| | | (s, 3H) |

The biochemical in vitro inhibitory activity of compounds disclosed herein were measured against an EGFR L858R/C797S mutant (EGFR LR/CS), and EGFR exon 19 deletion/C797S mutant (d19/CS) as described below. The inhibitory activity of compounds disclosed herein were measured in BaF3 cells expressing wild-type (WT) EGFR, am EGFR L858R/C797S mutant (EGFR LR/CS), and an EGFR exon 19 deletion/C797S mutant (d19/CS) as described below.

### Biological Activity Assay B: Inhibitory Activity of Compounds in a Biochemical Assay Against EGFR L858R/C797S Protein (“EGFR LR/CS” in Table 3)

The inhibitory activity of the compounds disclosed herein were measured in a biochemical assay utilizing a recombinant human EGFR L858R/C797S double mutant protein, utilizing detection of chelation-enhanced fluorescence using the sulfonamido-oxine (Sox) chromophore covalently attached to an optimized kinase substrate.

The following materials were used: EGFR L858R C797S (Carna Biosciences, Cat #08-563); phosphosens peptide substrate (AssayQuant, Cat #AQT0794); adenosine triphosphate (ThermoFisher, Cat #R0441); assay Buffer: 50 mM HEPES, pH=7.5, 10 mM MgCl₂, 1 mM EGTA, 0.01% BSA, 0.01% Brij-35, 1 mM DTT; 384-well Polypropylene Plates (Greiner Cat #781201); 384-well Black Low Volume Plates (Greiner Cat #784076); TopSeal-A Plus (PerkinElmer Cat #6050185); Dimethyl Sulfoxide (Sigma, Cat #D2650); 1 M HEPES (VWR Chemicals, Cat #J848); 2M Magnesium Chloride (Quality Biological, Cat #340-034-721); 0.5M Ethylene Glycol Tetra Acetic Acid (Alfa Aesar, Cat #J60767); 30% Bovine Albumin, Fraction V (Alfa Aesar, Cat #165569); 10% Brij-35, (EMD Millipore, Cat #203728); 1M Dithiothreitol, (Invitrogen, Cat #P2325)

In Row A of a 384-well polypropylene plate, was added 48 μL of DMSO. In wells A1 and A24, were added 12 μL of DMSO. Column 1 (16 replicates) served as enzyme+DMSO total signal (0% inhibition). Column 24 (16 replicates) served as no enzyme+DMSO background (100% inhibition). To a well in Row A were added 12 μL of each 10 mM test compound stock (100% DMSO). The master compound plate was transferred to the Bravo and run the “PPAR FRET cmpd serial dilution and intermediate dilution.pro” protocol to generate 16-point serial dilutions (3.16× or 0.5 log dilutions) of each compound. The final intermediate dilution plate contained 4×16-point compound serial dilutions in assay buffer. The DMSO concentration was 10%. The intermediate compound dilution plate was transferred to the Bravo and the “5 uL dispense into ARP.pro” protocol was run. To the black low volume assay ready plates, were added 5 μL of 40 uM Phosphosens peptide sensor/4 mM ATP working solution to each well. The plates were spun for 1 minute at 1200 rpm in an Eppendorf 5810R table-top centrifuge. Next, 10 μl of 2.0 nM EGFR L858R/C797S working solution was added to all wells in columns 1-23. To column 24, was added 10 μL of assay buffer. The final DMSO concentration was 2.5%. The plates were spun for 1 minute at 1200 rpm in an Eppendorf 5810R table-top centrifuge. The final concentrations of EGFR L858R/C797S, Phosphosens peptide substrate and ATP were 1 nM, 10 μM and 1 mM respectively. The plates were sealed with the TopSeal-A Plus plate seals. The plates were read on a BMG Clariostar (AssayQuant protocol) or Tecan Spark (AssayQuant_Phosphosens protocol) in kinetic mode for 3 h with 2 min intervals using the following settings: Excitation 360-20 nm, Dichroic auto 426, Emission 492-20 nm. Set for 90 cycles with 120 second interval. Raw data files containing 90 data points/well were exported from the plate readers in Excel and uploaded into Dotmatics using the “Thermo low conc EGFR L858R/C797S” protocol. The slope of each curve was constructed using Dotmatics in each well, and each IC₅₀s was calculated using a 4-parameter fit model.

Biological Activity Assay C: Inhibitory Activity of Compounds in a Biochemical Assay Against EGFR Exon 19 Deletion/C797S Protein (“EGFR d19/CS” in Table 3)

The inhibitory activity of the compounds disclosed herein were measured in a biochemical assay utilizing a recombinant human EGFR exon 19 deletion/C797S double mutant protein, utilizing detection of chelation-enhanced fluorescence using the sulfonamido-oxine (Sox) chromophore covalently attached to an optimized kinase substrate.

The following materials were used: EGFR d19-C797S (Carna Biosciences, Cat #08-564) phosphosens peptide substrate (AssayQuant, Cat #AQT0794); adenosine triphosphate (ThermoFisher, Cat #R0441); assay Buffer: 50 mM HEPES, pH=7.5, 10 mM MgCl₂, 1 mM EGTA, 0.01% BSA, 0.01% Brij-35, 1 mM DTT; 384-well Polypropylene Plates (Greiner Cat #781201); 384-well Black Low Volume Plates (Greiner Cat #784076); TopSeal-A Plus (PerkinElmer Cat #6050185); Dimethyl Sulfoxide (Sigma, Cat #D2650); 1 M HEPES (VWR Chemicals, Cat #J848); 2M Magnesium Chloride (Quality Biological, Cat #340-034-721); 0.5M Ethylene Glycol Tetra Acetic Acid (Alfa Aesar, Cat #J60767); 30% Bovine Albumin, Fraction V (Alfa Aesar, Cat #165569); 10% Brij-35, (EMD Millipore, Cat #203728); 1M Dithiothreitol, (Invitrogen, Cat #P2325)

In Row A of a 384-well polypropylene plate, was added 48 μL of DMSO. In wells A1 and A24, were added 12 μL of DMSO. Column 1 (16 replicates) served as enzyme+DMSO total signal (0% inhibition). Column 24 (16 replicates) served as no enzyme+DMSO background (100% inhibition). To a well in Row A were added 12 μL of each 10 mM test compound stock (100% DMSO). The master compound plate was transferred to the Bravo and run the “PPAR FRET cmpd serial dilution and intermediate dilution.pro” protocol to generate 16-point serial dilutions (3.16× or 0.5 log dilutions) of each compound. The final intermediate dilution plate contained 4×16-point compound serial dilutions in assay buffer. The DMSO concentration was 10%. The intermediate compound dilution plate was transferred to the Bravo and the “5 uL dispense into ARP.pro” protocol was run. To the black low volume assay ready plates, were added 5 μL of 40 uM Phosphosens peptide sensor/4 mM ATP working solution to each well. The plates were spun for 1 minute at 1200 rpm in an Eppendorf 5810R table-top centrifuge. Next, 10 μl of 2.0 nM EGFR d19/C797S working solution was added to all wells in columns 1-23. To column 24, was added 10 μL of assay buffer. The final DMSO concentration was 2.5%. The plates were spun for 1 minute at 1200 rpm in an Eppendorf 5810R table-top centrifuge. The final concentrations of EGFR d19/C797S, Phosphosens peptide substrate and ATP were 0.25 nM, 10 μM and 1 mM respectively. The plates were sealed with the TopSeal-A Plus plate seals. The plates were read on a BMG Clariostar (AssayQuant protocol) or Tecan Spark (AssayQuant_Phosphosens protocol) in kinetic mode for 3 h with 2 min intervals using the following settings: Excitation 360-20 nm, Dichroic auto 426, Emission 492-20 nm. Set for 90 cycles with 120 second interval. Raw data files containing 90 data points/well were exported from the plate readers in Excel and uploaded into Dotmatics using the Carna low conc EGFR d19/C797S protocol. The slope of each curve was constructed using Dotmatics in each well, and each IC50s was calculated using a 4-parameter fit model.

Biological Activity Assay D: Inhibitory Activity of Compounds in a BaF3 Assay Against EGFR Wild-Type (“BaF3 WT” in Table 3), L858R/C797s Mutant Protein (“BaF3 d19/CS” in Table 3), and Exon 19 Deletion/C797S Protein (“BaF3 d19/CS” in Table 3)

The inhibitory effect of compounds disclosed herein on cell proliferation (CP) was measured in isogenic Ba/F3 cell lines carrying either EGFR wild type (WT), EGFR LR_C797S or d19_C797S mutations.

The following materials were used: Cell Line: Ba/F3_EGFR WT, BaF3_EGFR d19/CS and BaF3_EGFR LR/CS were engineered using non-viral vectors to express WT EGFR, d19/CS EGFR or LR/CS EGFR, respectively; Culture Media: RPMI+10% FBS+2 mM L-Glut+100 μg/mL Primocin (or 1% Pen/Strep)+10 ng/mL human EGF+0.5 μg/mL puromycin; RPMI1640 with phenol red (Corning, Cat #: 15-040-CV); Fetal Bovine Serum (FBS) (Omega Scientific, Cat #: FB-11); Human Epidermal Growth Factor (hEGF) (Cell Signaling Cat #8916SC); L-Glutamine (Corning, Cat #: 25-005-CI); Penicillin/Streptomycin Solution, 100× (Corning, Cat #: 30-002-CI); Primocin (invivogen, Cat #: ant-pm-1; Assay Media: Same as culture medium. DMSO (Sigma, Cat #: D8418); 96-well Non-Sterile Polypropylene V Bottom Plates (Corning, Cat #: 3363); 384-well Sterile White with Flat Clear Bottom Plates (Greiner, 781098); CellTiter-Glo® 2.0 Cell Viability Assay (Promega, Cat #G9243).

One frozen vial of cells should be thawed into one T25 flask in 10 mL media. Keep the flask upright in the incubator.

Method: One frozen vial of cells was thawed into one T25 flask in 10 mL media and the flask was kept upright in the incubator. Cells were split 1:10 every two to three days when cells reached confluency of around 1×10⁶ cells/mL. Bravo protocol files were used with Greiner 384-well plates for all steps of compound preparation, cell plating and compound addition. Eight compounds per plate were tested at varying concentrations (generally 5-0 μM or 20-0 μM) with 1/3 serial dilutions, each concentration run in quadruplet in a 384-well plate. 16 μL of DMSO were added (for compounds starting at 20 μM) or 28 μL DMSO (for compounds starting at 5 μM) to 96-well V-bottom polypropylene plate: wells 1A-1H (8 compounds/plate). 16 μL 10 mM compound DMSO solution were added (for compounds starting at 20 μM) or 4 μL (for compounds starting at 5 μM) to column 1 for a starting concentration of 2.5 mM or 0.625 mM. 20 μL DMSO were added to the remainder of the wells (columns 2-12) in the 96-well V-bottom plate. A Bravo liquid handler was used to make 1:3 serial dilutions from columns 1 to 11 (12 remains as DMSO only) to prepare the DMSO dilution plate. 144 μL media were added to each well of a new 96-well V-bottom polypropylene plate to prepare the intermediate dilution plate. A Bravo or 96-head Integra liquid handler was used to add and mix 6 μL of test compound from the DMSO dilution plate to the intermediate dilution plate. (1:25 dilution). Cells were transferred from a flask into a 50 mL conical, vortex gently and the number of cells were counted, with a target cell number of about 1×10⁶ cells per mL. The cells were diluted in culture medium in 50 mL conical or T25 flask, and the cell suspension was added to BRAVO reservoir and then use Bravo protocol to plate the appropriate number of cells shown below in 36 μl of medium per well as follows: Ba/F3-WT-EGFR 2000 cells per well of a 384 well plate; Ba/F3-LR/CS-EGFR 2000 cells per well of a 384 well plate; and Ba/F3-d19/CS-EGFR 1500 cells per well of a 384 well plate. An extra 10 mL of dead volume was utilized in each reservoir.

Using a Bravo liquid handler, 4 μl of test compound solution was added per well from the intermediate dilution plate to plated cells (1:10 dilution from the intermediate plate, final compound dilution 1:250 and final DMSO concentration per well is 0.4%). Using the 96 BRAVO Head, 25 μl were aspirated from the intermediate dilution plate and 4 μl each were dispensed into 4 wells (square pattern) in the 384-well plate. Any remaining test compound is dispensed into a secondary plate to prevent cross contamination of cells in the intermediate compound plate. After the compounds were added, the plate was shaken at 600 rpm gently for 60 see, and the plates were incubated in a tissue culture incubator, 37° C., 5% CO₂, for ˜ 72 hours. After incubation, CTG 2.0 reagent was allowed to equilibrate to room temperature (˜3 hours) and cells to reach room temperature (˜10-15 minutes) and reagent was added to cells (40 μL/well) using a Bravo liquid handler. The plates were shaken to allow proper mixing (˜5 min) and let settle for 10 minutes and were read on Tecan plate reader using the appropriate luminescence method. Data from plate reader was transferred to an excel file and entered directly into Dotmatics using the protocols under EGFR Project/Cell Proliferation/named: “BaF3_EGFRwt_CP”, “BaF3_EGFR_d19/CS_CP”, “BaF3_EGFR_LR/CS_CP”. Curves were fit with a 4-parameter model and IC₅₀s calculated by the Dotmatics software. DMSO wells were averaged to define the high control (100% growth). The average of the highest concentration of the positive control compound (afatinib) was used to define the 0% growth and used to lock the bottom of the curves. The tops of the curves were not fixed. The IC₅₀s were calculated as the compound concentration that gives 50% growth.

TABLE 3

| Example | EGFR LR/CS | EGFR d19/CS | BaF3 LR/CS | BaF3 d19/CS | BaF3 WT |
|---|---|---|---|---|---|
| No. | IC₅₀ (nM) | IC₅₀ (nM) | IC₅₀ (nM) | IC₅₀ (nM) | IC₅₀ (nM) |
| 1 | N/A | N/A | 349 | 210.8 | 2568 |
| 2 | N/A | N/A | 89.49 | 103.7 | 723.7 |
| 3 | 3.149 | 1.662 | 130.1 | 169 | 807.7 |
| 4 | 1.293 | 0.4664 | 32.13 | 36.44 | 1403 |
| 5 | 1.201 | 0.4497 | 80.31 | 90.63 | 2433 |
| 6 | 0.2527 | 0.148 | 4.402 | 5.814 | 1104 |
| 7 | N/A | N/A | 694.2 | 508.4 | 4713 |
| 8 | 1.092 | 1.138 | 19.64 | 19.63 | 753.7 |
| 9 | 0.099 | 0.121 | 0.6301 | 0.5471 | 15.44 |
| 10 | 0.152 | 0.113 | 1.627 | 1.315 | 74.05 |
| 11 | 0.5206 | 0.408 | 6.905 | 5.793 | 594.2 |
| 12 | 1.079 | 0.7322 | 25.32 | 21.36 | 483.2 |
| 13 | 0.272 | 0.1734 | 4.1 | 3.708 | 255.1 |
| 14 | 7.575 | 3.299 | 120.2 | 113.3 | 1354 |
| 15 | 0.2702 | 0.2072 | 1.025 | 0.9485 | 64.73 |
| 16 | 1.47 | 1.57 | 4.384 | 3.391 | 815.8 |
| 17 | 8.44 | 3.461 | 153.3 | 157.1 | 1744 |
| 18 | 0.1409 | 0.0926 | 2.057 | 1.834 | 71.43 |
| 19 | 0.2425 | 0.1697 | 0.4017 | 0.2441 | 8.669 |
| 20 | 4.915 | 4.192 | 577.9 | 685.5 | 5337 |
| 21 | 5.2 | 5.474 | 375.2 | 539.4 | 18510 |
| 22 | 196.8 | 90.89 | 3660 | 4104 | 20000 |
| 23 | 1.096 | 0.6334 | 69.93 | 31.92 | 2649 |
| 24 | 1.13 | 0.9089 | 124.5 | 44.66 | 2491 |
| 25 | 12.99 | 7.724 | 726 | 810.4 | 3486 |
| 26 | 4.062 | 4.223 | 562.4 | 486.4 | 6411 |
| 27 | 8.647 | 3.118 | 208.5 | 464.1 | 11710 |
| 28 | 10.76 | 4.158 | 493.3 | 884.6 | 17820 |
| 29 | 0.1107 | 0.0769 | 1.294 | 1.217 | 88.38 |
| 30 | 1.715 | 1.877 | 260.5 | 319.1 | 4438 |
| 31 | 0.5209 | 0.4462 | 97.94 | 66.45 | 3325 |
| 32 | 1.356 | 1.257 | 129.6 | 113.6 | 4729 |
| 33 | 0.2118 | 0.1191 | 1.155 | 1.08 | 37.95 |
| 34 | 1.169 | 1.594 | 225.2 | 122 | 7784 |
| 35 | 0.9579 | 0.5399 | 134.5 | 184.8 | 5947 |
| 36 | 1.127 | 1.346 | 208.8 | 339.4 | 20000 |
| 37 | 1.866 | 2.595 | 317.9 | 514.4 | 6275 |
| 38 | 0.4338 | 0.8559 | 120.7 | 44.8 | 2172 |
| 39 | 6.145 | 5.186 | 161.1 | 147.3 | 1923 |
| 40 | 0.327 | 0.192 | 36.4 | 22 | 2097 |
| 41 | 0.518 | 0.375 | 61.64 | 34.73 | 2388 |
| 42 | 0.527 | 0.335 | 57.47 | 37.52 | 2093 |
| 43 | 0.39 | 0.24 | 73.17 | 40.05 | 2241 |
| 44 | 2.764 | 1.786 | 262.6 | 421 | 6654 |
| 45 | 1.12 | 1.509 | 185.1 | 37.48 | 2289 |
| 46 | 0.3894 | 0.3169 | 41.15 | 21.09 | 2155 |
| 47 | 0.594 | 0.473 | 92.45 | 72.47 | 4530 |
| 48 | 0.4 | 0.374 | 60.38 | 47.92 | 2206 |
| 49 | 3.415 | 1.766 | 132.2 | 249.9 | 20000 |
| 50 | 0.635 | 1.017 | 240.6 | 59.15 | 2078 |
| 51 | 17.12 | 27.23 | 770.3 | 972.1 | 10690 |
| 52 | 0.307 | 0.368 | 106.4 | 20.78 | 1943 |
| 53 | 0.462 | 0.538 | 121.8 | 40.84 | 2130 |
| 54 | 15.8 | 20.08 | 992.4 | 836.9 | 8863 |
| 55 | 0.247 | 0.29 | 78.53 | 21.42 | 1284 |
| 56 | 3.578 | 4.583 | 223.5 | 184.1 | 5492 |
| 57 | 16.84 | 10.35 | 689.2 | 1294 | 7387 |
| 58 | 0.349 | 0.177 | 58.16 | 43.71 | 2101 |
| 59 | 1.269 | 0.752 | 235.6 | 212.7 | 5948 |
| 60 | 0.526 | 0.419 | 88.26 | 118.5 | 2267 |
| 61 | 0.601 | 0.907 | 73.2 | 24.4 | 1139 |
| 62 | 0.527 | 0.703 | 45 | 30.1 | 1148 |
| 63 | 0.474 | 0.459 | 35.4 | 48.5 | 1274 |
| 64 | 0.779 | 1.104 | 99.1 | 62.9 | 1457 |
| 65 | 0.263 | 0.341 | 24.3 | 19.9 | 589 |
| 66 | 14.54 | 8.121 | 2686 | 1291 | >20000 |
| 67 | 15.64 | 18.61 | 1629 | 1051 | 8042 |
| 68 | 5.489 | 2.252 | 203 | 723 | N/A |
| 69 | 0.452 | 0.378 | 40.2 | 68.6 | 2339 |
| 70 | 0.328 | 0.346 | 7.9 | 11.5 | 681 |
| 71 | 1.254 | 1.621 | 84.1 | 151 | 2625 |
| 72 | 0.65 | 0.36 | 26.1 | 24.2 | 1335 |
| 73 | 0.77 | 0.48 | 80.9 | 56 | 2492 |
| 74 | 9.37 | 2.9 | 267 | 920 | >20000 |
| 75 | 1.01 | 1.49 | 131 | 150 | 5915 |
| 76 | 0.94 | 0.7 | 45.5 | 31 | 2025 |
| 77 | 1.36 | 1.61 | 139 | 72.2 | 5179 |
| 78 | 0.48 | 0.37 | 23.2 | 18.4 | 2730 |
| 79 | 2.64 | 1.99 | 116 | 111 | 10210 |
| 80 | 6.38 | 3.25 | 143 | 388 | N/A |
| 81 | 2.58 | 1.12 | 241 | 330 | 13510 |
| 82 | 4.33 | 2.86 | 392 | 597 | >20000 |
| 83 | 6.32 | 8.54 | 529 | 640 | >20000 |
| 84 | 6.21 | 10.8 | 566 | 779 | >20000 |
| 85 | 0.58 | 0.39 | 27.5 | 33.8 | 1859 |
| 86 | 6.25 | 6.67 | 390 | 429 | 7383 |
| 87 | 1.56 | 1.34 | 140 | 196 | 2814 |
| 88 | 1.22 | 0.64 | 301 | 238 | 3737 |
| 89 | 0.38 | 0.3 | 19.9 | 20.4 | 373 |
| 90 | 0.51 | 0.24 | 36.2 | 64.9 | 1508 |
| 91 | 0.37 | 0.23 | 23.8 | 39.9 | 1276 |
| 92 | 0.95 | 0.84 | 231 | 255 | 6218 |
| 93 | 0.91 | 0.73 | 184 | 450 | 4291 |
| 94 | 0.17 | 0.11 | 22.4 | 27.4 | 1116 |
| 95 | 0.75 | 0.32 | 116 | 95.2 | 2088 |
| 96 | 0.39 | 0.62 | 73.8 | 150 | 1478 |
| 97 | 0.33 | 0.34 | 39.2 | 79.4 | 1187 |
| 98 | 0.82 | 1.27 | 161 | 212 | 1861 |
| 99 | 5.7 | 8.51 | 822 | 701 | >20000 |
| 100 | 2.76 | 4.23 | 407 | 319 | 13320 |
| 101 | 0.39 | 0.27 | 8.69 | 4.96 | 115 |
| 102 | 0.35 | 0.2 | 21.1 | 16.5 | 161 |
| 103 | 0.16 | 0.13 | 8.35 | 17.5 | 229 |
| 104 | 1.67 | 0.46 | 174 | 98 | 10550 |
| 105 | 0.75 | 1.69 | 136 | 152 | 1697 |
| 106 | 0.45 | 0.38 | 55.7 | 64.7 | 1016 |
| 107 | 9.83 | 6.94 | 610 | 314 | 3370 |
| 108 | 1.59 | 0.45 | 130 | 105 | 1525 |
| 109 | 0.4 | 0.18 | 15.7 | 23.6 | 667 |
| 110 | 0.69 | 0.33 | 68.7 | 83.1 | 1377 |
| 111 | 0.45 | 0.41 | 53.7 | 114 | 1136 |
| 112 | 12.9 | 12.5 | 741 | 1147 | 7149 |
| 113 | 1.27 | 1.49 | 256 | 315 | 2187 |
| 114 | 1.62 | 0.55 | 132 | 137 | 3316 |
| 115 | 0.41 | 0.31 | 31.6 | 110 | 703 |
| 116 | 0.37 | 0.23 | 22.9 | 47.6 | 1511 |
| 117 | 0.47 | 0.27 | 103 | 93.7 | 1271 |
| 118 | 0.56 | 0.25 | 38.3 | 50.5 | 1024 |
| 119 | 0.7 | 0.34 | 35.5 | 50.9 | 1341 |
| 120 | N/A | 0.12 | 8.82 | 17.1 | 633 |
| 121 | N/A | 0.14 | 12.6 | 29.8 | 1176 |
| 122 | N/A | 0.55 | 68 | 113.9 | 1846 |
| 123 | N/A | 2.89 | 96.1 | 260 | 3886 |
| 124 | N/A | 3.02 | 230 | 611 | 4274 |
| 125 | N/A | 0.21 | 11.8 | 52.1 | 741 |
| 126 | N/A | 0.18 | 14.6 | 58.2 | 720 |
| 127 | N/A | 0.09 | 15.8 | 24.6 | 3680 |
| 128 | N/A | 0.41 | 105 | 125 | 1738 |
| 129 | N/A | 0.2 | 66.6 | 107 | 1606 |
| 130 | N/A | 0.12 | 10.1 | 12 | 2345 |
| 131 | N/A | 1.04 | 81.9 | 162 | 2999 |
| 132 | N/A | 1.51 | 1157 | 860 | 5489 |
| 133 | N/A | 7.1 | 2186 | 1191 | 7551 |
| 134 | N/A | 0.3 | 33.3 | 57.1 | 1031 |
| 135 | N/A | 0.33 | 17.1 | 31.9 | 1157 |
| 136 | N/A | 0.97 | 81.5 | 127 | 3050 |
| 137 | N/A | 0.69 | 40.5 | 71.3 | 2496 |
| 138 | N/A | 0.5 | 94.4 | 63.7 | 1390 |
| 139 | N/A | 8.55 | 574 | 551 | 10309 |
| 140 | N/A | 3.32 | 1632 | 916 | 5682 |
| 141 | N/A | 0.25 | 43.6 | 66.1 | 921 |
| 142 | N/A | 5.19 | 313 | 398 | 6074 |
| 143 | N/A | 0.38 | 59.8 | 94.3 | 663 |
| 144 | N/A | 2.41 | 33.6 | 89.6 | 1608 |
| 145 | N/A | 0.55 | 83.6 | 114 | 3206 |
| 146 | N/A | 4.18 | 927.8 | 525.4 | 7878 |
| 147 | N/A | 0.26 | 73.27 | 68.57 | 1383 |
| 148 | N/A | 0.61 | 69.76 | 92.35 | 1507 |
| 149 | N/A | 3.72 | 2770 | 1155 | >20,000 |
| 150 | N/A | 1.42 | 42.19 | 69.89 | 3317 |
| 151 | N/A | 1.45 | 16.15 | 68.11 | 1869 |
| 152 | N/A | 1.33 | 22.52 | 88.94 | 2022 |
| 153 | N/A | 1.04 | 22.26 | 60.41 | 1799 |
| 154 | N/A | 0.75 | 629.6 | 439.8 | 16300 |
| 155 | N/A | 0.41 | 64.98 | 63.79 | 1164 |
| 156 | N/A | 0.31 | 18.77 | 30.51 | 1481 |
| 157 | N/A | 0.57 | 15.23 | 40.78 | 1588 |
| 158 | N/A | 0.26 | 15.13 | 33.02 | 782.1 |
| 159 | N/A | 3.79 | 511.2 | 267.4 | 6049 |
| 160 | N/A | 4.99 | 751.3 | 359.2 | 14210 |
| 161 | N/A | 0.14 | 4.93 | 13.05 | 564.9 |
| 162 | N/A | 0.44 | 10.55 | 63.87 | 1363 |
| 163 | N/A | 0.27 | 10.13 | 22.06 | 619.2 |
| 164 | N/A | 0.29 | 26.34 | 36.33 | 887.6 |
| 165 | N/A | 0.26 | 20.6 | 37.02 | 1093 |
| 166 | N/A | 0.29 | 19.56 | 53.83 | 1349 |
| 167 | N/A | 0.83 | 32.84 | 161.7 | 1641 |
| 168 | N/A | 15.59 | 3572 | 1147 | >20,000 |
| 169 | N/A | 3.08 | 399 | 461.2 | 11320 |
| 170 | N/A | 1.37 | 97.13 | 213.4 | 4839 |
| 171 | N/A | 0.06 | 7.74 | 26.59 | 1812 |
| 172 | N/A | 1.44 | 33.12 | 198.9 | 7048 |
| 173 | N/A | 0.11 | 17.64 | 28.95 | 800.2 |
| 174 | N/A | 3.72 | 770.5 | 490.9 | 9073 |
| 175 | N/A | 6.08 | 472.2 | 248 | 11700 |
| 176 | N/A | 0.36 | 134.1 | 139.9 | 2544 |
| 177 | N/A | 0.42 | 42.02 | 56.41 | 1594 |
| 178 | N/A | 2.27 | 89.74 | 186.9 | 5200 |
| 179 | N/A | 0.39 | 17.21 | 41.02 | 2197 |
| 180 | N/A | 0.39 | 127.8 | 160.6 | 2924 |
| 181 | N/A | 0.27 | 79.29 | 97.03 | 2108 |
| 182 | N/A | 1.65 | 443.6 | 391.5 | >20,000 |
| 183 | N/A | 0.14 | 144.8 | 145.7 | 5420 |
| 184 | N/A | 0.26 | 30.7 | 57.56 | 1769 |
| 185 | N/A | 1.43 | 255.5 | 395.2 | 6977 |
| 186 | N/A | 0.63 | 185.7 | 348.4 | 4447 |
| 187 | N/A | 3.36 | 1236 | 588.6 | 9025 |
| 188 | N/A | 2.23 | 488.9 | 245.9 | 11700 |
| 189 | N/A | 0.26 | 5.51 | 5.86 | 131.5 |
| 190 | N/A | 0.18 | 63.95 | 59.09 | 2161 |
| 191 | N/A | 0.65 | >20,000 | >20,000 | >20,000 |
| 192 | N/A | 0.97 | 195.7 | 260.2 | 3072 |
| 193 | N/A | 1.64 | 173.7 | 258.8 | 3041 |
| 194 | N/A | 0.83 | 174.7 | 273.5 | 3612 |
| 195 | N/A | 6.89 | 798.7 | 364.8 | 6002 |
| 196 | N/A | 0.89 | 212.3 | 133 | 1740 |
| 197 | N/A | 0.45 | 12.78 | 46.45 | 138.6 |
| 198 | N/A | 0.72 | 17.93 | 51.85 | 702.7 |
| 199 | N/A | 0.92 | 85.44 | 294.1 | 3337 |
| 200 | N/A | 0.13 | 5.04 | 8.78 | 412 |
| 201 | N/A | 0.52 | 200.3 | 141.5 | 1577 |
| 202 | N/A | 1.48 | 219.4 | 511.9 | 2354 |
| 203 | N/A | 0.14 | 10.55 | 17.06 | 288.7 |
| 204 | N/A | 10.19 | 288.2 | 1223 | 8128 |
| 205 | N/A | 0.11 | 51.05 | 49.7 | 1281 |
| 206 | N/A | 6.02 | 2505 | 1108 | >20,000 |
| 207 | N/A | 3.26 | 140.2 | 632.7 | 4392 |
| 208 | N/A | 0.4 | 154.6 | 95.71 | 1681 |
| 209 | N/A | 0.44 | 295.5 | 160.2 | 1937 |
| 210 | N/A | 0.5 | 61.06 | 92.23 | 1694 |
| 211 | N/A | 0.35 | 18.27 | 20.15 | 885.8 |
| 212 | N/A | 0.37 | 6.71 | 5.28 | 87.78 |
| 213 | N/A | 0.51 | 80.91 | 168 | 2607 |
| 214 | N/A | 0.88 | 135.8 | 302.8 | 2507 |
| 215 | N/A | 0.26 | 30.83 | 83.49 | 2336 |
| 216 | N/A | 0.06 | 31.14 | 58.45 | 2127 |
| 217 | N/A | 0.276 | 146.9 | 98.9 | 1243 |
| 218 | N/A | 0.36 | 158 | 103 | 1444 |
| 219 | N/A | 1.53 | 221.2 | 200.4 | 4374 |
| 220 | N/A | 0.55 | 37.19 | 110.6 | 1038 |
| 221 | N/A | 0.33 | 20.67 | 51.77 | 709.5 |
| 222 | N/A | 0.59 | 76.75 | 182.2 | 1253 |
| 223 | N/A | 21.6 | 2203 | 893.2 | >20,000 |
| 224 | N/A | 0.14 | 26.63 | 37.69 | 1450 |
| 225 | N/A | 4.88 | 502.5 | 885.8 | 18940 |
| 226 | N/A | 1.86 | 236.3 | 412.6 | 10200 |
| 227 | N/A | 0.63 | 53.27 | 42.32 | 2666 |
| 228 | N/A | 2.13 | 666.8 | 384 | 4558 |
| 229 | N/A | 0.67 | 291.8 | 169.2 | 3765 |
| 230 | N/A | 5.1 | 88.46 | 287 | 2369 |
| 231 | N/A | 0.46 | 17.95 | 35.37 | 660.2 |
| 232 | N/A | 0.23 | 9.42 | 9.99 | 300.9 |
| 233 | N/A | 4.09 | 227.7 | 414.3 | 6040 |
| 234 | N/A | 3.6 | 437.6 | 308.8 | 2121 |
| 235 | N/A | 0.24 | 41.78 | 41.29 | 836.8 |
| 236 | N/A | 0.16 | 11.17 | 17.1 | 924.3 |
| 237 | N/A | 0.74 | 35.02 | 73.92 | 2496 |
| 238 | N/A | 0.62 | 31 | 52.14 | 2070 |
| 239 | N/A | 1.73 | 1495 | 1282 | 12450 |
| 240 | N/A | 0.34 | 57.89 | 66.4 | 7559 |
| 241 | N/A | 1.2 | 188.7 | 130.6 | 1515 |
| 242 | N/A | 0.18 | 8.34 | 10.76 | 533.3 |
| 243 | N/A | 0.37 | 93.04 | 87.84 | 1199 |
| 244 | N/A | 0.55 | 91.71 | 96.25 | 1044 |
| 245 | N/A | 0.31 | 82.07 | 69.86 | 3210 |
| 246 | N/A | 0.06 | 10.61 | 26.94 | 875.5 |
| 247 | N/A | 0.09 | 8.8 | 26.93 | 1363 |
| 248 | N/A | 0.42 | 100 | 92.74 | 2549 |
| 249 | N/A | 0.25 | 103.4 | 103.5 | 4514 |
| 250 | N/A | 0.05 | 3.54 | 9.26 | 696.3 |
| 251 | N/A | 0.2 | 8.08 | 12.8 | 744.6 |
| 252 | N/A | 0.09 | 8.87 | 21.87 | 630 |
| 253 | N/A | 0.2 | 51.6 | 87.66 | 7741 |
| 254 | N/A | 0.11 | 3.05 | 5.01 | 442 |
| 255 | N/A | 0.47 | 50.95 | 32.44 | 1696 |
| 256 | N/A | 0.19 | 5.23 | 11.22 | 403.2 |
| 257 | N/A | 0.2 | 18.21 | 20.75 | 1012 |
| 258 | N/A | 0.16 | 12.68 | 18.53 | 976.1 |
| 259 | N/A | 0.1 | 4.44 | 14.42 | 497.5 |
| 260 | N/A | 0.35 | 17.01 | 33.01 | 1921 |
| 261 | N/A | 0.206 | 4.527 | 10.06 | 550 |
| 262 | N/A | 0.396 | 78.24 | 47.04 | 4062 |
| 263 | N/A | 0.153 | 2.359 | 6.174 | 234.6 |
| 264 | N/A | 0.135 | 6.543 | 8.851 | 448.1 |
| 265 | N/A | 0.078 | 1.433 | 1.462 | 69.87 |
| 266 | N/A | 0.094 | 2.131 | 2.503 | 135.2 |
| 267 | N/A | 0.185 | 22.2 | 15.97 | 687.6 |
| 268 | N/A | 0.104 | 16.52 | 13.35 | 413.6 |
| 269 | N/A | 0.082 | 8.631 | 7.718 | 391.9 |
| 270 | N/A | 0.227 | 1.878 | 1.325 | 133.5 |
| 271 | N/A | 0.252 | 20.61 | 14.12 | 1604 |
| 272 | N/A | 0.486 | 13.47 | 15.72 | 668.2 |
| 273 | N/A | 0.291 | 22.87 | 24.77 | 833.5 |
| 274 | N/A | 0.213 | 16.09 | 23.47 | 720.6 |
| 275 | N/A | 0.207 | 13.38 | 11.9 | 551.3 |
| 276 | N/A | 0.07 | 18.98 | 13.46 | 677 |
| 277 | N/A | 0.332 | 74.41 | 40.01 | 1501 |
| 278 | N/A | 0.123 | 5.141 | 4.306 | 272.2 |
| 279 | N/A | 0.106 | 8.887 | 5.643 | 475.6 |
| 280 | N/A | 0.288 | 38.25 | 31.7 | 856.8 |
| 281 | N/A | 0.425 | 80.7 | 57.12 | 1838 |
| 282 | N/A | 0.307 | 62.9 | 40.71 | 1983 |
| 283 | N/A | 0.113 | 9.793 | 9.467 | 656.6 |
| 284 | N/A | 0.203 | 11.21 | 10.15 | 1004 |
| 285 | N/A | 0.228 | 4.863 | 3.745 | 321.4 |
| 286 | N/A | 0.203 | 20.15 | 15.77 | 1113 |
| 287 | N/A | 0.213 | 8.635 | 8.854 | 613.1 |

1. A compound of Formula (XII), 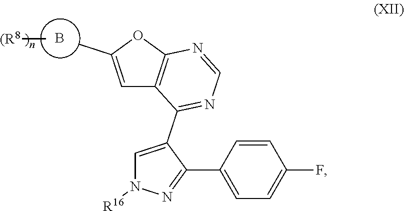
wherein:
B is aryl, heteroaryl, heterocycloalkyl, or C₃-C₁₂ cycloalkyl;
each R⁸ is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, heterocycloalkenyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1a};
R¹⁶ is selected from hydrogen, deuterium, halogen, —CN, oxo, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, —OC₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{1b};
each R^{1a} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NR^{b}S(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl;
or two R^{1a} on the same atom are taken together to form an oxo;
each R^{1b} is independently selected from deuterium, halogen, —CN, —NO₂, —OH, —OR^{a}, —OC(═O)R^{a}, —OC(═O)OR^{b}, —OC(═O)NR^{c}R^{d}, —SH, —SR^{a}, —S(═O)R^{a}, —S(═O)₂R^{a}, —S(═O)₂NR^{c}R^{d}, —NR^{c}R^{d}, —NR^{b}C(═O)NR^{c}R^{d}, —NR^{b}C(═O)R^{a}, —NR^{b}C(═O)OR^{a}, —NRS(═O)₂R^{a}, —C(═O)R^{a}, —C(═O)OR^{b}, —C(═O)NR^{c}R^{d}, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl; wherein each C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, and heteroaryl is optionally and independently substituted with one or more R^{a};
or two R^{1b} on the same atom are taken together to form an oxo;
each R^{a} and R^{b} is independently selected from hydrogen, deuterium, halogen, —CN, —NO₂, —OH, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C₃-C₁₀cycloalkyl), C₁-C₆alkyl(heterocycloalkyl), C₁-C₆alkyl(C6-C₁₀aryl), and C₁-C₆alkyl(heteroaryl);
wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
each R^{c} and R^{d} are independently selected from hydrogen, C₁-C₆alkyl, C₁-C₆haloalkyl, C₁-C₆deuteroalkyl, C₁-C₆hydroxyalkyl, C₁-C₆aminoalkyl, C₁-C₆heteroalkyl, C₂-C₆alkenyl, C₂-C₆alkynyl, C₃-C₁₀cycloalkyl, heterocycloalkyl, C₆-C₁₀aryl, heteroaryl, C₁-C₆alkyl(C3-C₁₀cycloalkyl), C₁-C₆alkyl(heterocycloalkyl), C₁-C₆alkyl(C6-C₁₀aryl), and C₁-C₆alkyl(heteroaryl); wherein each of the C₁-C₆alkyl, C₂-C₆alkenyl, C₂-C₆alkynyl, C₃-C₁₀cycloalkyl, heterocycloalkyl, C₆-C₁₀aryl, and heteroaryl is independently optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl;
or R^{c} and R^{d} are taken together with the atom to which they are attached to form a heterocycloalkyl optionally substituted with one or more oxo, deuterium, halogen, —CN, —OH, —OCH₃, —S(═O)CH₃, —S(═O)₂CH₃, —S(═O)₂NH₂, —S(═O)₂NHCH₃, —S(═O)₂N(CH₃)₂, —NH₂, —NHCH₃, —N(CH₃)₂, —C(═O)CH₃, —C(═O)OH, —C(═O)OCH₃, C₁-C₆alkyl, C₁-C₆haloalkyl, C₁-C₆deuteroalkyl, C₁-C₆hydroxyalkyl, C₁-C₆aminoalkyl, or C₁-C₆heteroalkyl; and
n is 0, 1, 2, 3, 4, or 5; or a pharmaceutically acceptable salt thereof.

2. A compound according to claim 1, or a pharmaceutically acceptable salt thereof, wherein B is aryl.

3. A compound according to claim 1, or a pharmaceutically acceptable salt thereof, wherein B is heteroaryl.

4. A compound according to claim 1, or a pharmaceutically acceptable salt thereof, wherein B is heterocycloalkyl.

5. A compound according to claim 1, or a pharmaceutically acceptable salt thereof, wherein B is C₃-C₁₂ cycloalkyl.

6. A compound according to any one of claims 1 to 5, or a pharmaceutically acceptable salt thereof, wherein n is 1, 2, or 3.

7. A compound according to claim 6, or a pharmaceutically acceptable salt thereof, wherein n is 1.

8. A compound according to claim 3, or a pharmaceutically acceptable salt thereof, wherein B is pyridinyl, pyrazolyl, pyrimidinyl, pyrazinyl, pyridazinyl, imidazolyl, thiazolyl, isothiazolyl, oxazolyl, isoxazolyl, oxadiazolyl, triazolyl, thiadiazolyl, or tetrazolyl.

9. A compound according to claim 4, or a pharmaceutically acceptable salt thereof, wherein B is 3,6-dihydropyridinyl, piperazinyl, tetrahydropyridinyl, piperidinyl, tetrahydrofuranyl, dihydropyranyl, tetrahydropyranyl, dioxalanyl, or 2-azaspiro[3.4]octan-2-yl.

10. A compound according to any one of claims 1 to 9, or a pharmaceutically acceptable salt thereof, wherein R¹⁶ is selected from ethyl, 1,1-dioxo-1λ6-thietan-3-yl, 4-butanamidyl, 1-methylpyrrolidin-2-onyl, 3-fluorocyclobutyl, 3-fluorooxan-4-yl, 3-fluoropropyl, 3,3-difluoropropyl, 2,2-dimethylpropanamidinyl, 1-imino-1,6-thiolan-1-onyl, 1-methylcyclobutan-1-ol, 3,3-difluorocyclobutyl, 1,6-thiolane-1,1-dionyl, 1-(3-fluorooxan-4-yl), 1-(methanesulfonyl)-5-methylpyrrolidin-3-yl, 2-hydroxy-2-methylpropyl, 2-fluorocyclohexyl, 3-(methanesulfonyl)propyl, 4,4-dimethyloxetan-2-yl)methyl, 5,5-dimethyloxolan-3-yl, 1-[2-(difluoromethoxy)]ethyl, 3-cyanopropyl, 3-(methanesulfonyl)cyclobutyl, cyclobutan-1-ol, cyclobutane-1-carbonitrile, 2,2-difluoropropan-1-ol, and 2,2-difluorocyclopropyl.

11. A compound selected from the group consisting of: 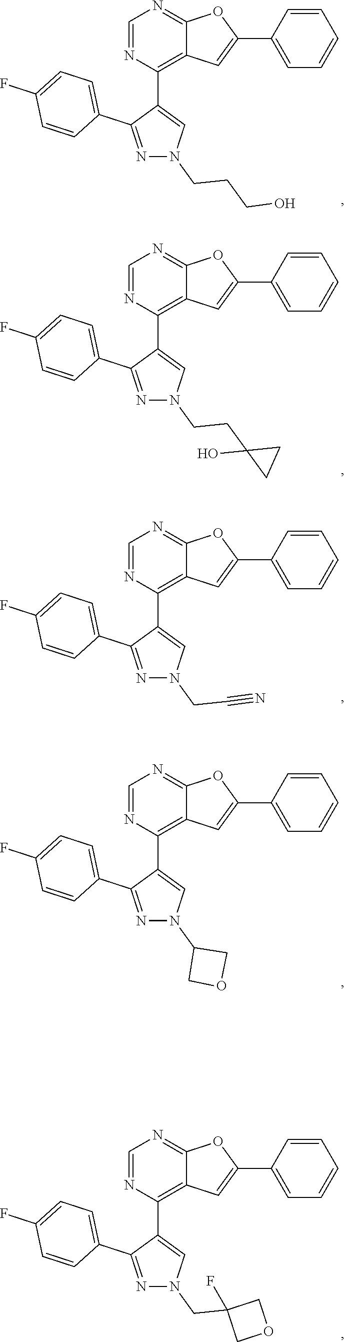 [SMILES: OCCCn1cc(-c2ncnc3oc(-c4ccccc4)cc23)c(-c2ccc(F)cc2)n1 ; OC1(CCn2cc(-c3ncnc4oc(-c5ccccc5)cc34)c(-c3ccc(F)cc3)n2)CC1 ; N#CCn1cc(-c2ncnc3oc(-c4ccccc4)cc23)c(-c2ccc(F)cc2)n1 ; Fc1ccc(-c2nn(C3COC3)cc2-c2ncnc3oc(-c4ccccc4)cc23)cc1 ; Fc1ccc(-c2nn(CC3(F)COC3)cc2-c2ncnc3oc(-c4ccccc4)cc23)cc1] 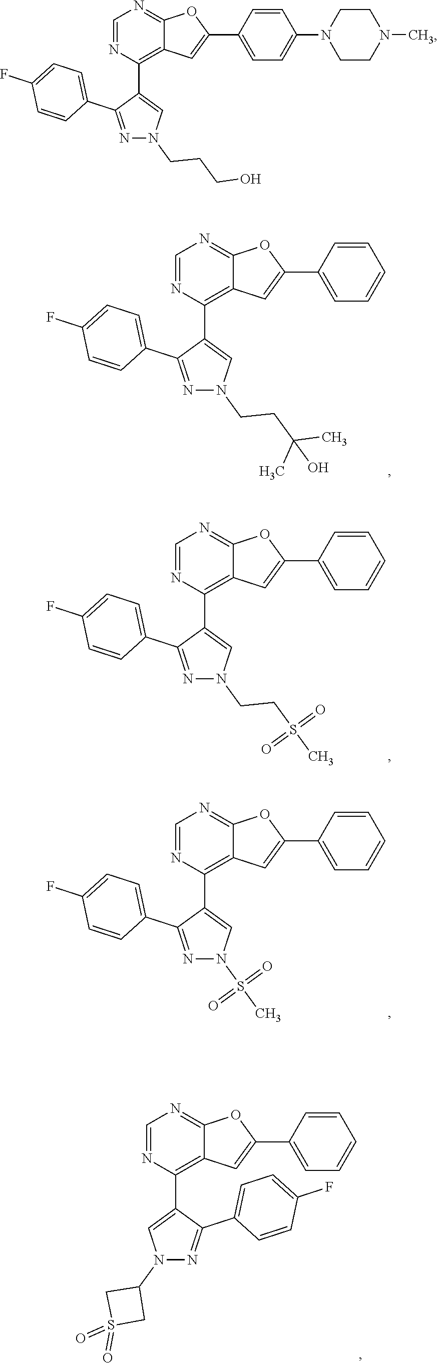 [SMILES: CN1CCN(c2ccc(-c3cc4c(-c5cn(CCCO)nc5-c5ccc(F)cc5)ncnc4o3)cc2)CC1 ; CC(C)(O)CCn1cc(-c2ncnc3oc(-c4ccccc4)cc23)c(-c2ccc(F)cc2)n1 ; CS(=O)(=O)CCn1cc(-c2ncnc3oc(-c4ccccc4)cc23)c(-c2ccc(F)cc2)n1 ; CS(=O)(=O)n1cc(-c2ncnc3oc(-c4ccccc4)cc23)c(-c2ccc(F)cc2)n1 ; O=S1(=O)CC(n2cc(-c3ncnc4oc(-c5ccccc5)cc34)c(-c3ccc(F)cc3)n2)C1] 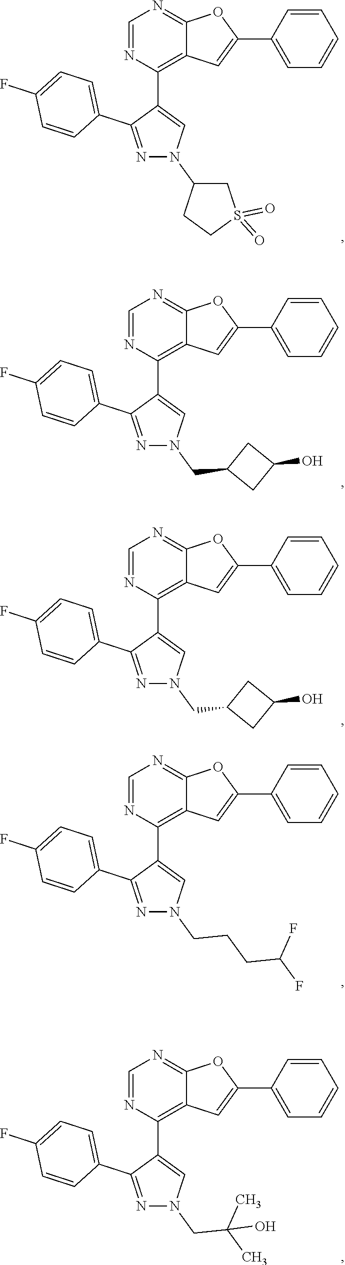 [SMILES: O=S1(=O)CCC(n2cc(-c3ncnc4oc(-c5ccccc5)cc34)c(-c3ccc(F)cc3)n2)C1 ; O[C@H]1C[C@@H](Cn2cc(-c3ncnc4oc(-c5ccccc5)cc34)c(-c3ccc(F)cc3)n2)C1 ; O[C@H]1C[C@H](Cn2cc(-c3ncnc4oc(-c5ccccc5)cc34)c(-c3ccc(F)cc3)n2)C1 ; Fc1ccc(-c2nn(CCCC(F)F)cc2-c2ncnc3oc(-c4ccccc4)cc23)cc1 ; CC(C)(O)Cn1cc(-c2ncnc3oc(-c4ccccc4)cc23)c(-c2ccc(F)cc2)n1] 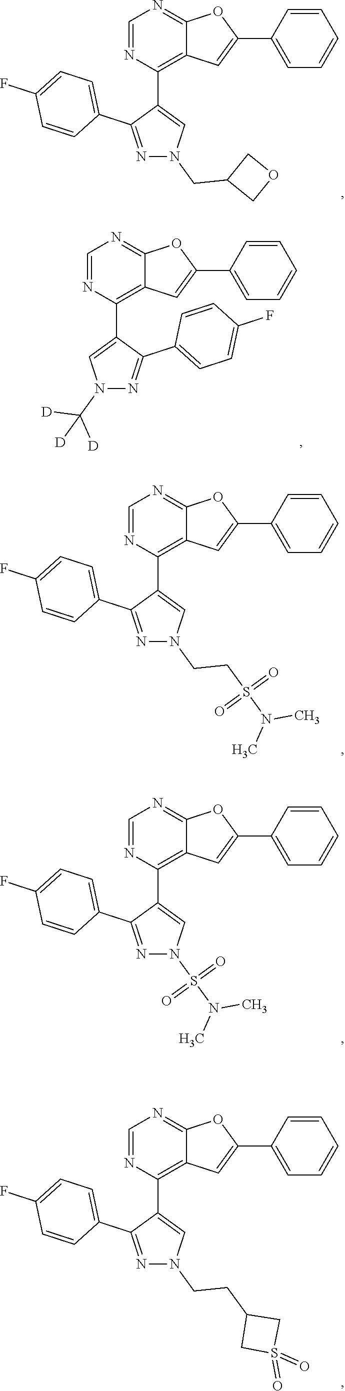 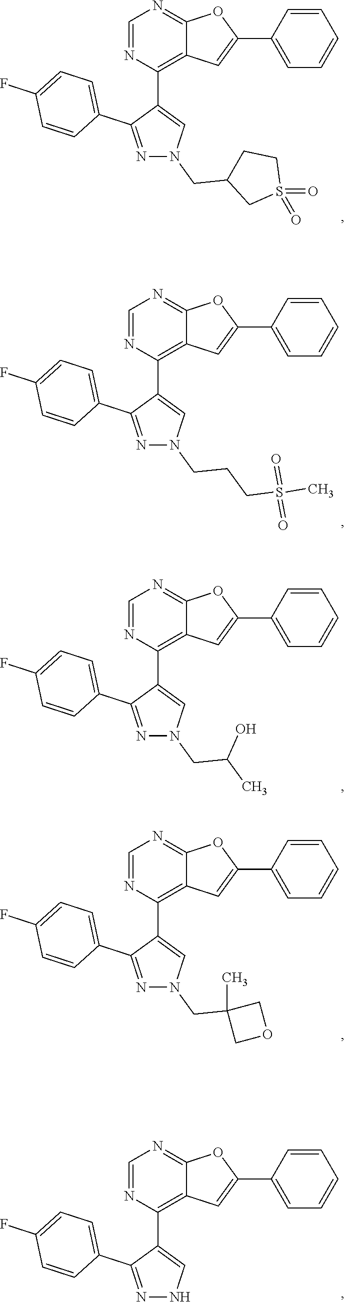 [SMILES: O=S1(=O)CCC(Cn2cc(-c3ncnc4oc(-c5ccccc5)cc34)c(-c3ccc(F)cc3)n2)C1 ; CS(=O)(=O)CCCn1cc(-c2ncnc3oc(-c4ccccc4)cc23)c(-c2ccc(F)cc2)n1 ; CC(O)Cn1cc(-c2ncnc3oc(-c4ccccc4)cc23)c(-c2ccc(F)cc2)n1 ; CC1(Cn2cc(-c3ncnc4oc(-c5ccccc5)cc34)c(-c3ccc(F)cc3)n2)COC1 ; Fc1ccc(-c2n[nH]cc2-c2ncnc3oc(-c4ccccc4)cc23)cc1] 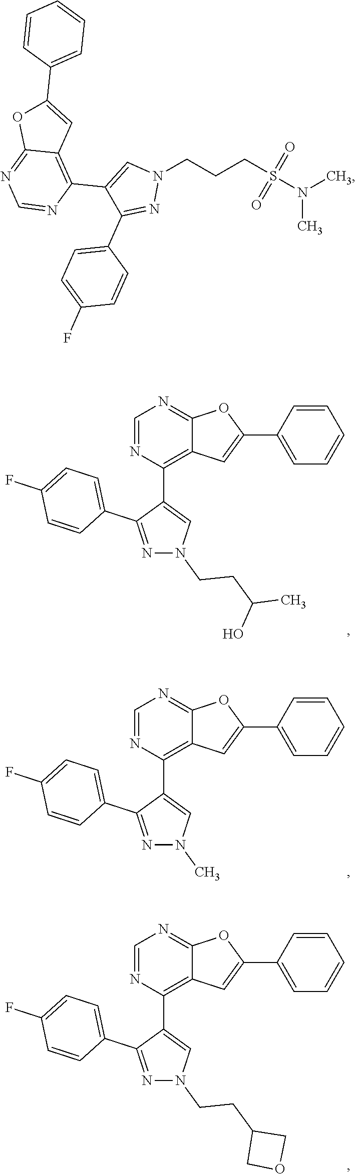 [SMILES: CN(C)S(=O)(=O)CCCn1cc(-c2ncnc3oc(-c4ccccc4)cc23)c(-c2ccc(F)cc2)n1 ; CC(O)CCn1cc(-c2ncnc3oc(-c4ccccc4)cc23)c(-c2ccc(F)cc2)n1 ; Cn1cc(-c2ncnc3oc(-c4ccccc4)cc23)c(-c2ccc(F)cc2)n1 ; Fc1ccc(-c2nn(CCC3COC3)cc2-c2ncnc3oc(-c4ccccc4)cc23)cc1] 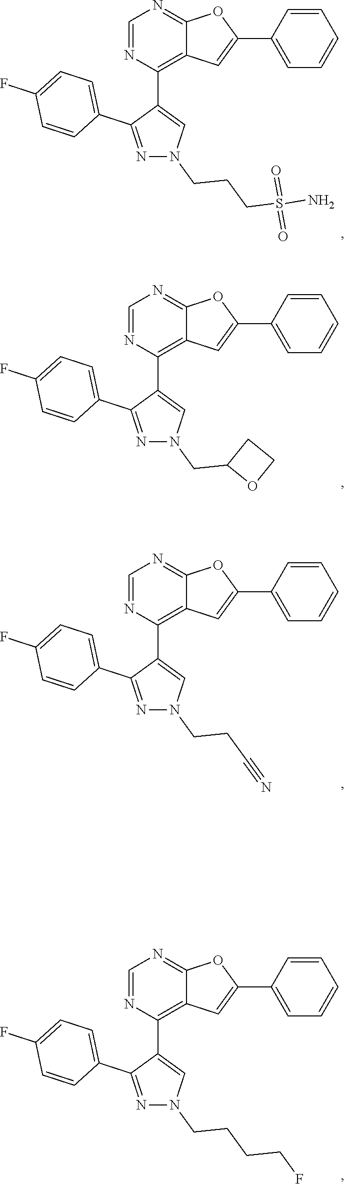 [SMILES: NS(=O)(=O)CCCn1cc(-c2ncnc3oc(-c4ccccc4)cc23)c(-c2ccc(F)cc2)n1 ; Fc1ccc(-c2nn(CC3CCO3)cc2-c2ncnc3oc(-c4ccccc4)cc23)cc1 ; N#CCCn1cc(-c2ncnc3oc(-c4ccccc4)cc23)c(-c2ccc(F)cc2)n1 ; FCCCCn1cc(-c2ncnc3oc(-c4ccccc4)cc23)c(-c2ccc(F)cc2)n1] 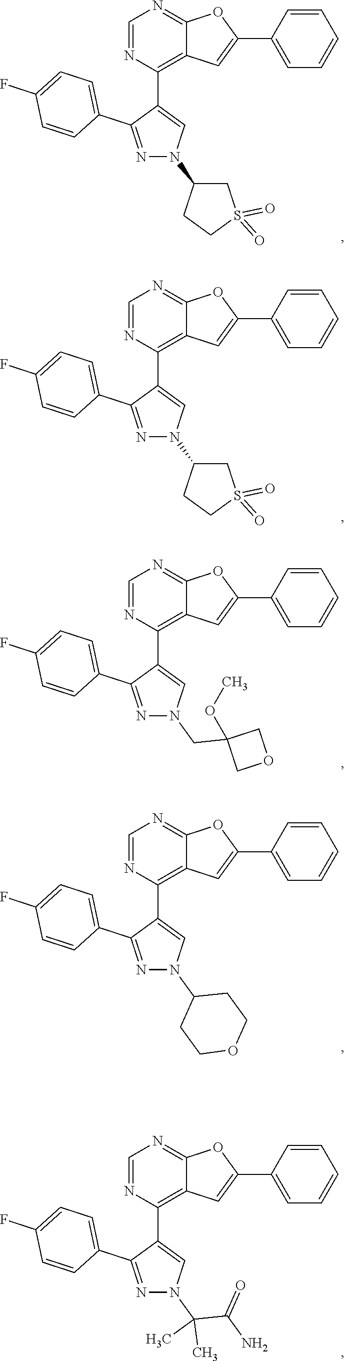 [SMILES: O=S1(=O)CC[C@@H](n2cc(-c3ncnc4oc(-c5ccccc5)cc34)c(-c3ccc(F)cc3)n2)C1 ; O=S1(=O)CC[C@H](n2cc(-c3ncnc4oc(-c5ccccc5)cc34)c(-c3ccc(F)cc3)n2)C1 ; COC1(Cn2cc(-c3ncnc4oc(-c5ccccc5)cc34)c(-c3ccc(F)cc3)n2)COC1 ; Fc1ccc(-c2nn(C3CCOCC3)cc2-c2ncnc3oc(-c4ccccc4)cc23)cc1 ; CC(C)(C(N)=O)n1cc(-c2ncnc3oc(-c4ccccc4)cc23)c(-c2ccc(F)cc2)n1] 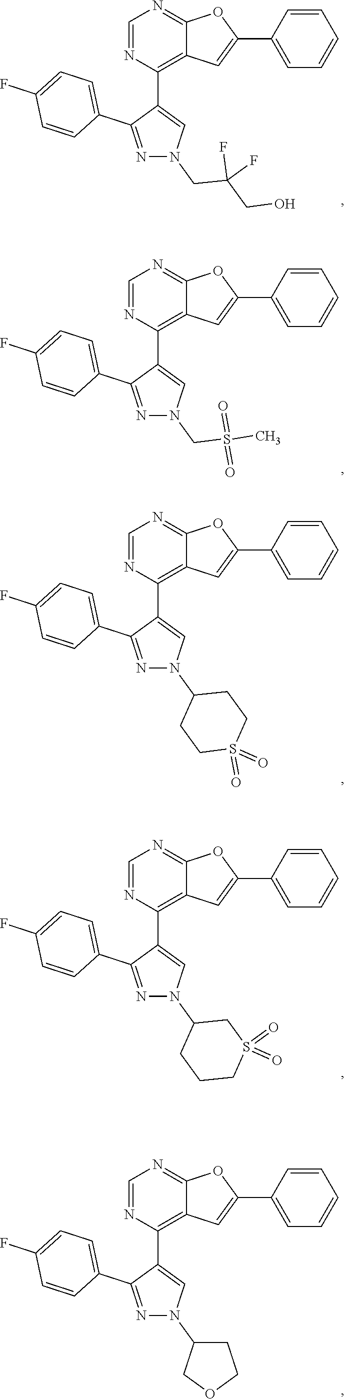 [SMILES: OCC(F)(F)Cn1cc(-c2ncnc3oc(-c4ccccc4)cc23)c(-c2ccc(F)cc2)n1 ; CS(=O)(=O)Cn1cc(-c2ncnc3oc(-c4ccccc4)cc23)c(-c2ccc(F)cc2)n1 ; O=S1(=O)CCC(n2cc(-c3ncnc4oc(-c5ccccc5)cc34)c(-c3ccc(F)cc3)n2)CC1 ; O=S1(=O)CCCC(n2cc(-c3ncnc4oc(-c5ccccc5)cc34)c(-c3ccc(F)cc3)n2)C1 ; Fc1ccc(-c2nn(C3CCOC3)cc2-c2ncnc3oc(-c4ccccc4)cc23)cc1] 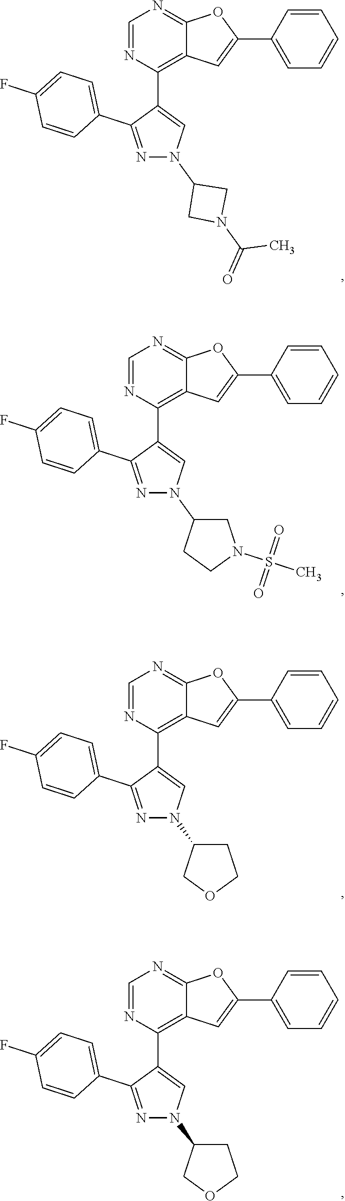 [SMILES: CC(=O)N1CC(n2cc(-c3ncnc4oc(-c5ccccc5)cc34)c(-c3ccc(F)cc3)n2)C1 ; CS(=O)(=O)N1CCC(n2cc(-c3ncnc4oc(-c5ccccc5)cc34)c(-c3ccc(F)cc3)n2)C1 ; Fc1ccc(-c2nn([C@@H]3CCOC3)cc2-c2ncnc3oc(-c4ccccc4)cc23)cc1 ; Fc1ccc(-c2nn([C@H]3CCOC3)cc2-c2ncnc3oc(-c4ccccc4)cc23)cc1] 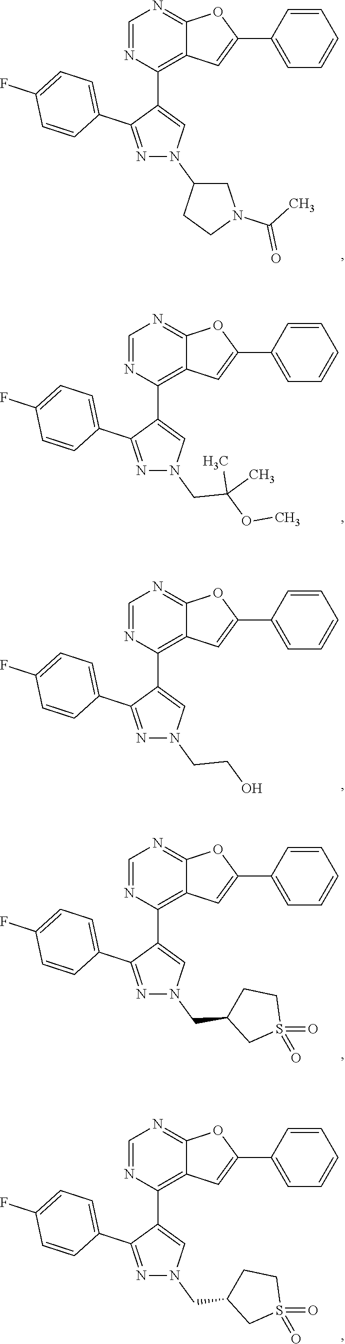 [SMILES: CC(=O)N1CCC(n2cc(-c3ncnc4oc(-c5ccccc5)cc34)c(-c3ccc(F)cc3)n2)C1 ; COC(C)(C)Cn1cc(-c2ncnc3oc(-c4ccccc4)cc23)c(-c2ccc(F)cc2)n1 ; OCCn1cc(-c2ncnc3oc(-c4ccccc4)cc23)c(-c2ccc(F)cc2)n1 ; O=S1(=O)CC[C@H](Cn2cc(-c3ncnc4oc(-c5ccccc5)cc34)c(-c3ccc(F)cc3)n2)C1 ; O=S1(=O)CC[C@@H](Cn2cc(-c3ncnc4oc(-c5ccccc5)cc34)c(-c3ccc(F)cc3)n2)C1] 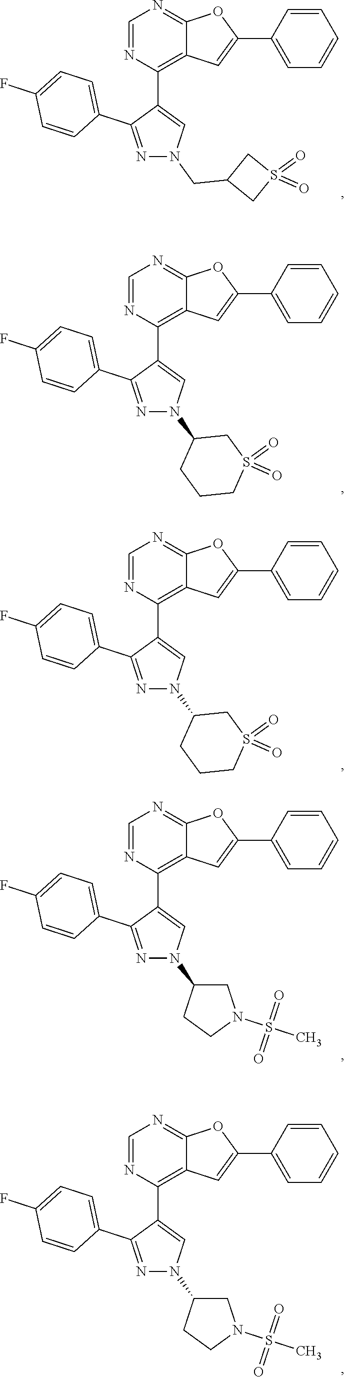 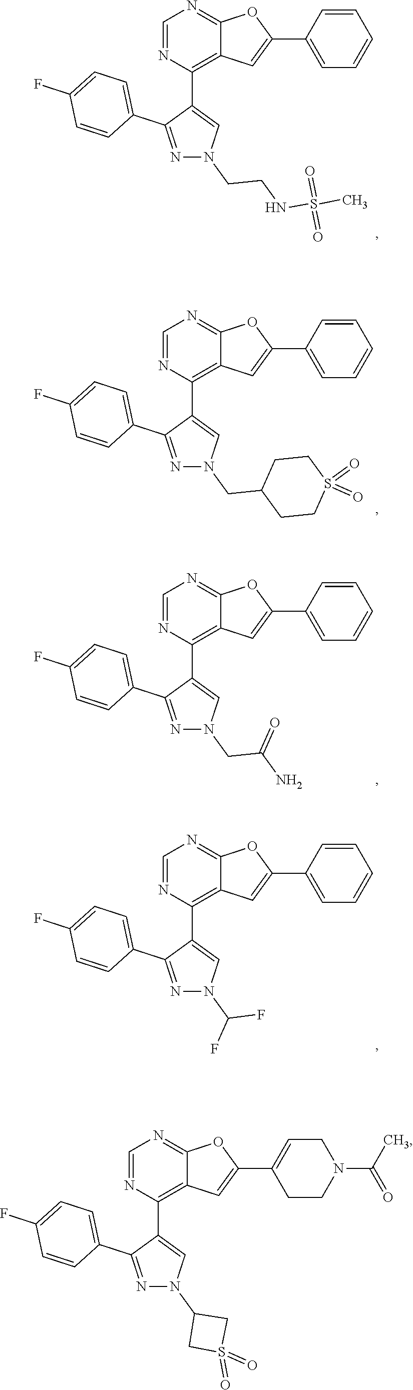 [SMILES: CS(=O)(=O)NCCn1cc(-c2ncnc3oc(-c4ccccc4)cc23)c(-c2ccc(F)cc2)n1 ; O=S1(=O)CCC(Cn2cc(-c3ncnc4oc(-c5ccccc5)cc34)c(-c3ccc(F)cc3)n2)CC1 ; NC(=O)Cn1cc(-c2ncnc3oc(-c4ccccc4)cc23)c(-c2ccc(F)cc2)n1 ; Fc1ccc(-c2nn(C(F)F)cc2-c2ncnc3oc(-c4ccccc4)cc23)cc1 ; CC(=O)N1CC=C(c2cc3c(-c4cn(C5CS(=O)(=O)C5)nc4-c4ccc(F)cc4)ncnc3o2)CC1] 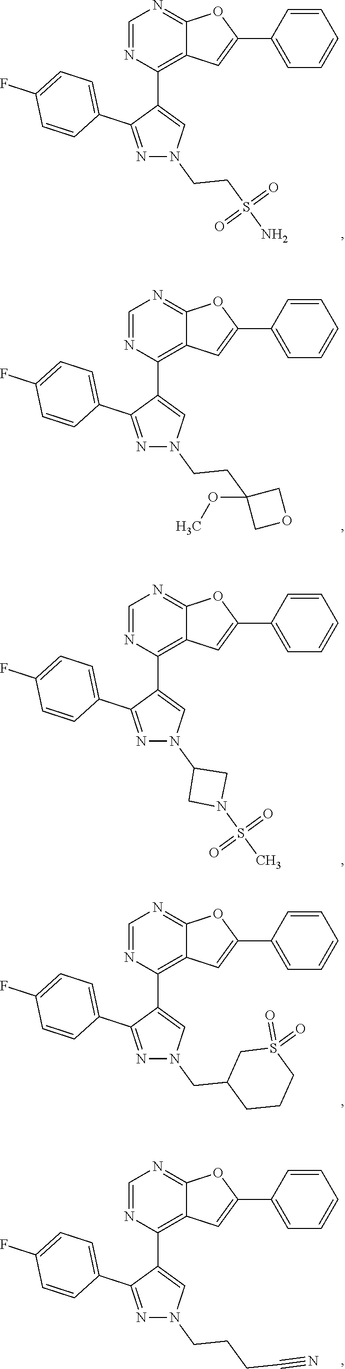 [SMILES: NS(=O)(=O)CCn1cc(-c2ncnc3oc(-c4ccccc4)cc23)c(-c2ccc(F)cc2)n1 ; COC1(CCn2cc(-c3ncnc4oc(-c5ccccc5)cc34)c(-c3ccc(F)cc3)n2)COC1 ; CS(=O)(=O)N1CC(n2cc(-c3ncnc4oc(-c5ccccc5)cc34)c(-c3ccc(F)cc3)n2)C1 ; O=S1(=O)CCCC(Cn2cc(-c3ncnc4oc(-c5ccccc5)cc34)c(-c3ccc(F)cc3)n2)C1 ; N#CCCCn1cc(-c2ncnc3oc(-c4ccccc4)cc23)c(-c2ccc(F)cc2)n1] 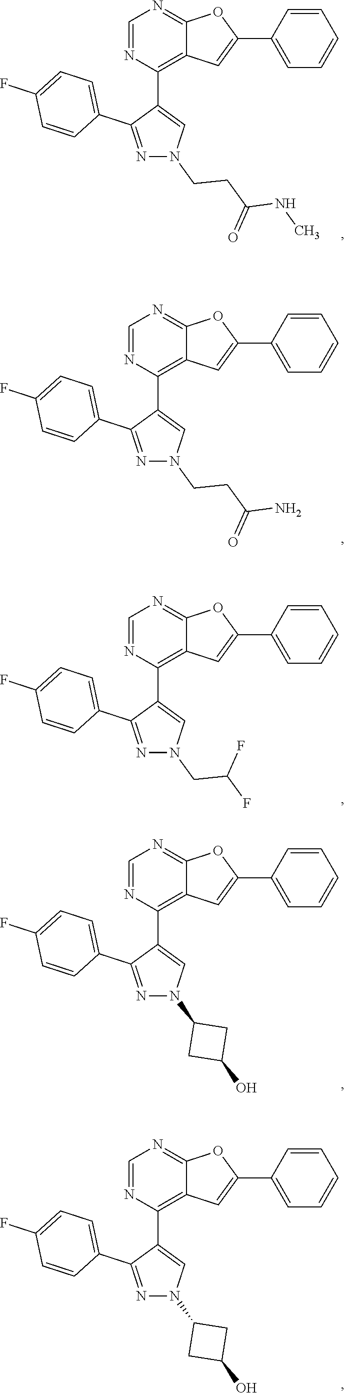 [SMILES: CNC(=O)CCn1cc(-c2ncnc3oc(-c4ccccc4)cc23)c(-c2ccc(F)cc2)n1 ; NC(=O)CCn1cc(-c2ncnc3oc(-c4ccccc4)cc23)c(-c2ccc(F)cc2)n1 ; Fc1ccc(-c2nn(CC(F)F)cc2-c2ncnc3oc(-c4ccccc4)cc23)cc1 ; O[C@H]1C[C@@H](n2cc(-c3ncnc4oc(-c5ccccc5)cc34)c(-c3ccc(F)cc3)n2)C1 ; O[C@H]1C[C@H](n2cc(-c3ncnc4oc(-c5ccccc5)cc34)c(-c3ccc(F)cc3)n2)C1] 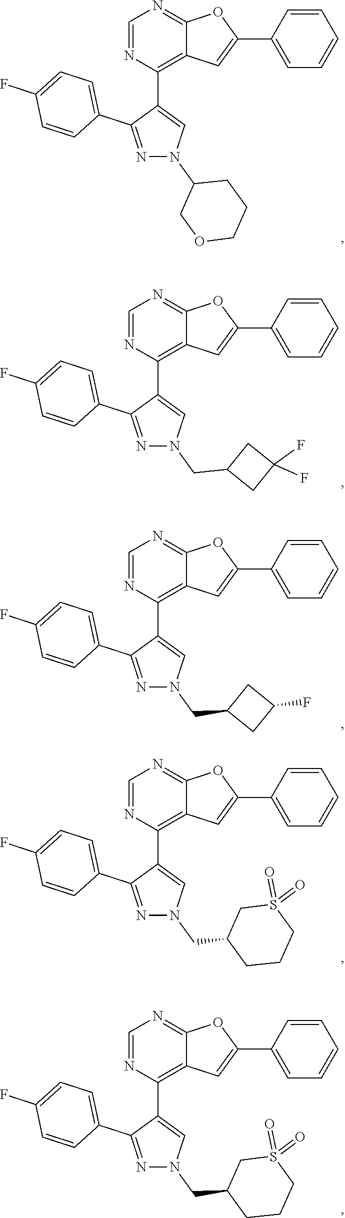 [SMILES: Fc1ccc(-c2nn(C3CCCOC3)cc2-c2ncnc3oc(-c4ccccc4)cc23)cc1 ; Fc1ccc(-c2nn(CC3CC(F)(F)C3)cc2-c2ncnc3oc(-c4ccccc4)cc23)cc1 ; Fc1ccc(-c2nn(C[C@H]3C[C@H](F)C3)cc2-c2ncnc3oc(-c4ccccc4)cc23)cc1 ; O=S1(=O)CCC[C@H](Cn2cc(-c3ncnc4oc(-c5ccccc5)cc34)c(-c3ccc(F)cc3)n2)C1 ; O=S1(=O)CCC[C@@H](Cn2cc(-c3ncnc4oc(-c5ccccc5)cc34)c(-c3ccc(F)cc3)n2)C1] 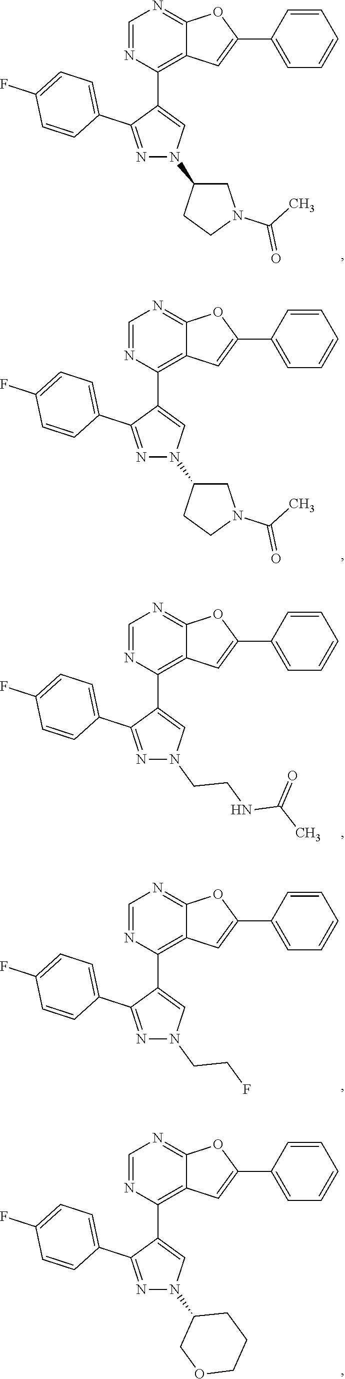 [SMILES: CC(=O)N1CC[C@@H](n2cc(-c3ncnc4oc(-c5ccccc5)cc34)c(-c3ccc(F)cc3)n2)C1 ; CC(=O)N1CC[C@H](n2cc(-c3ncnc4oc(-c5ccccc5)cc34)c(-c3ccc(F)cc3)n2)C1 ; CC(=O)NCCn1cc(-c2ncnc3oc(-c4ccccc4)cc23)c(-c2ccc(F)cc2)n1 ; FCCn1cc(-c2ncnc3oc(-c4ccccc4)cc23)c(-c2ccc(F)cc2)n1 ; Fc1ccc(-c2nn([C@@H]3CCCOC3)cc2-c2ncnc3oc(-c4ccccc4)cc23)cc1] 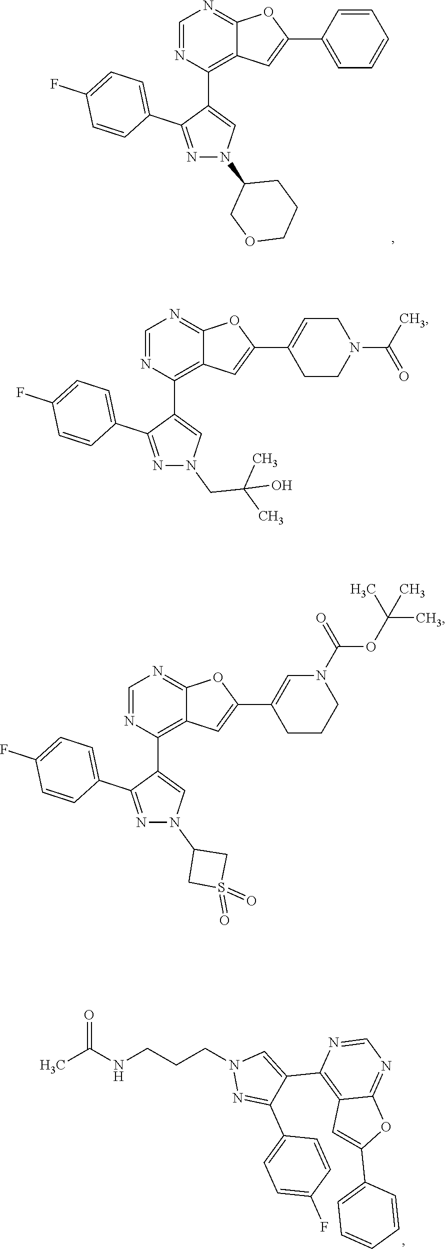 [SMILES: Fc1ccc(-c2nn([C@H]3CCCOC3)cc2-c2ncnc3oc(-c4ccccc4)cc23)cc1 ; CC(=O)N1CC=C(c2cc3c(-c4cn(CC(C)(C)O)nc4-c4ccc(F)cc4)ncnc3o2)CC1 ; CC(C)(C)OC(=O)N1C=C(c2cc3c(-c4cn(C5CS(=O)(=O)C5)nc4-c4ccc(F)cc4)ncnc3o2)CCC1 ; CC(=O)NCCCn1cc(-c2ncnc3oc(-c4ccccc4)cc23)c(-c2ccc(F)cc2)n1] 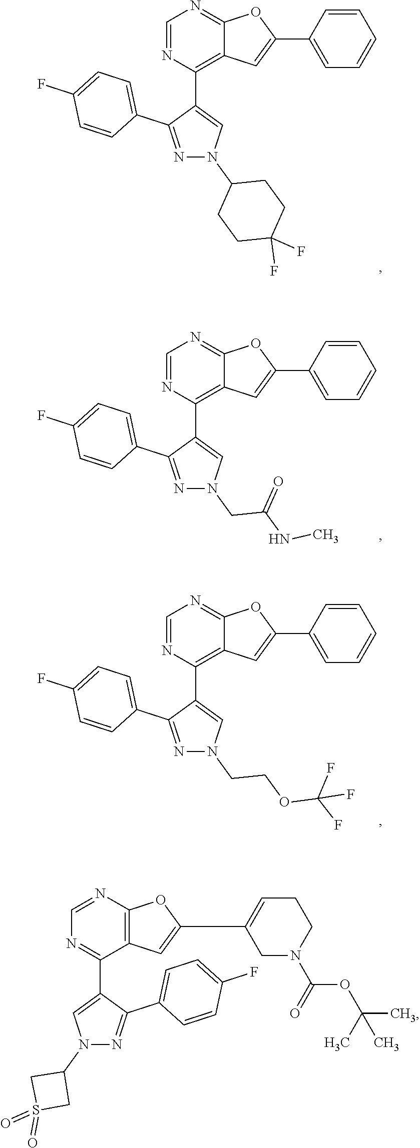 [SMILES: Fc1ccc(-c2nn(C3CCC(F)(F)CC3)cc2-c2ncnc3oc(-c4ccccc4)cc23)cc1 ; CNC(=O)Cn1cc(-c2ncnc3oc(-c4ccccc4)cc23)c(-c2ccc(F)cc2)n1 ; Fc1ccc(-c2nn(CCOC(F)(F)F)cc2-c2ncnc3oc(-c4ccccc4)cc23)cc1 ; CC(C)(C)OC(=O)N1CCC=C(c2cc3c(-c4cn(C5CS(=O)(=O)C5)nc4-c4ccc(F)cc4)ncnc3o2)C1] 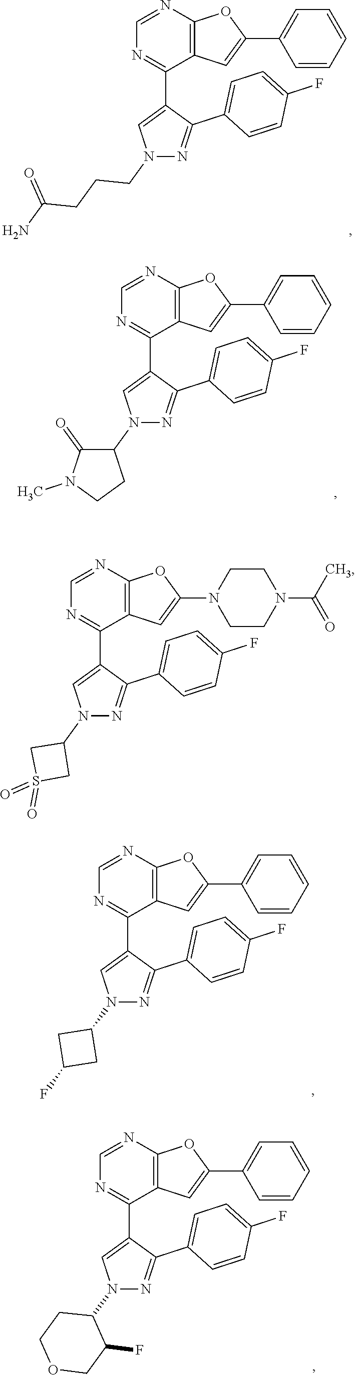 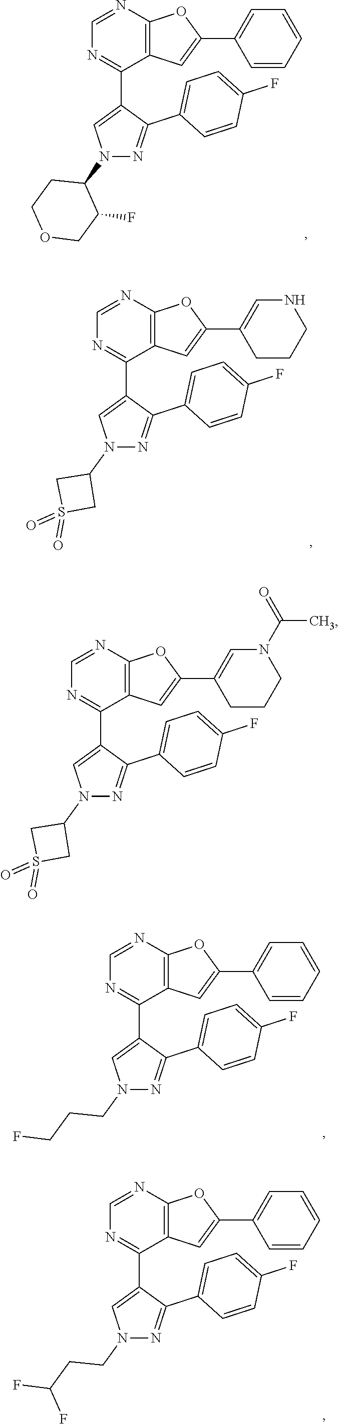 [SMILES: Fc1ccc(-c2nn([C@@H]3CCOC[C@H]3F)cc2-c2ncnc3oc(-c4ccccc4)cc23)cc1 ; O=S1(=O)CC(n2cc(-c3ncnc4oc(C5=CNCCC5)cc34)c(-c3ccc(F)cc3)n2)C1 ; CC(=O)N1C=C(c2cc3c(-c4cn(C5CS(=O)(=O)C5)nc4-c4ccc(F)cc4)ncnc3o2)CCC1 ; FCCCn1cc(-c2ncnc3oc(-c4ccccc4)cc23)c(-c2ccc(F)cc2)n1 ; Fc1ccc(-c2nn(CCC(F)F)cc2-c2ncnc3oc(-c4ccccc4)cc23)cc1] 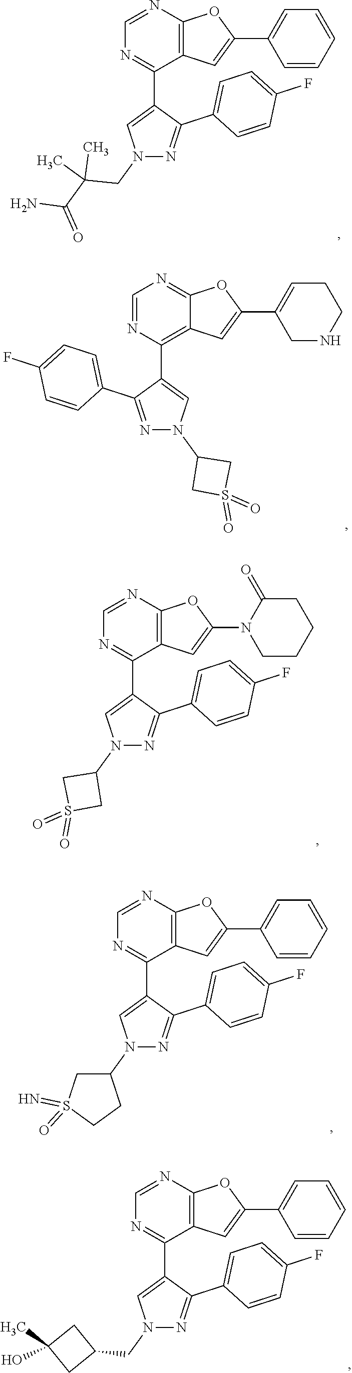 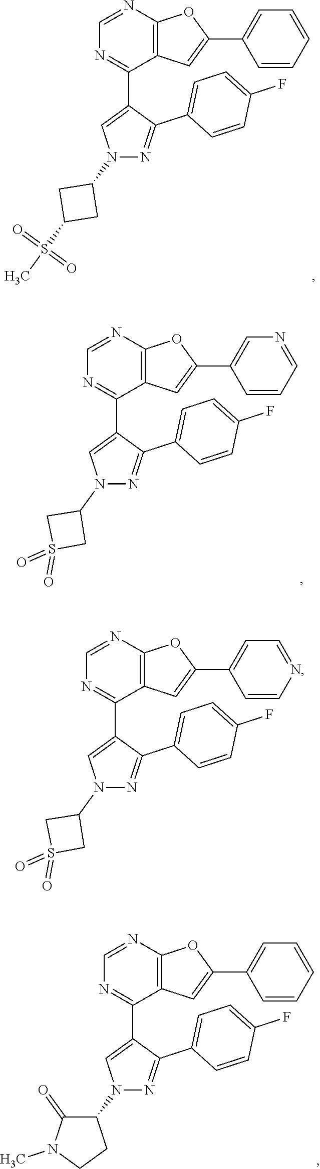 [SMILES: CS(=O)(=O)[C@H]1C[C@@H](n2cc(-c3ncnc4oc(-c5ccccc5)cc34)c(-c3ccc(F)cc3)n2)C1 ; O=S1(=O)CC(n2cc(-c3ncnc4oc(-c5cccnc5)cc34)c(-c3ccc(F)cc3)n2)C1 ; O=S1(=O)CC(n2cc(-c3ncnc4oc(-c5ccncc5)cc34)c(-c3ccc(F)cc3)n2)C1 ; CN1CC[C@@H](n2cc(-c3ncnc4oc(-c5ccccc5)cc34)c(-c3ccc(F)cc3)n2)C1=O] 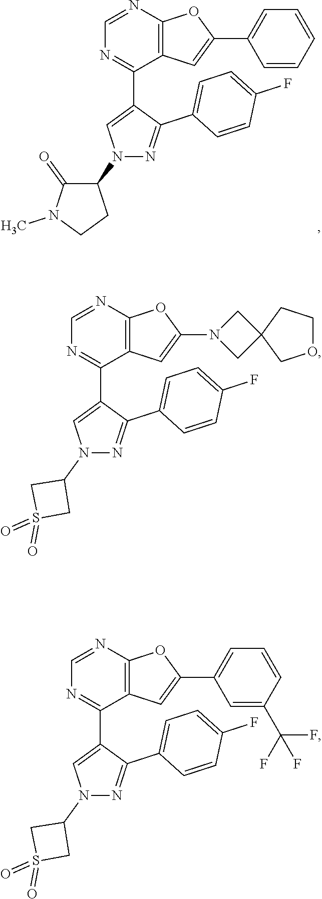 [SMILES: CN1CC[C@H](n2cc(-c3ncnc4oc(-c5ccccc5)cc34)c(-c3ccc(F)cc3)n2)C1=O ; O=S1(=O)CC(n2cc(-c3ncnc4oc(N5CC6(CCOC6)C5)cc34)c(-c3ccc(F)cc3)n2)C1 ; O=S1(=O)CC(n2cc(-c3ncnc4oc(-c5cccc(C(F)(F)F)c5)cc34)c(-c3ccc(F)cc3)n2)C1] 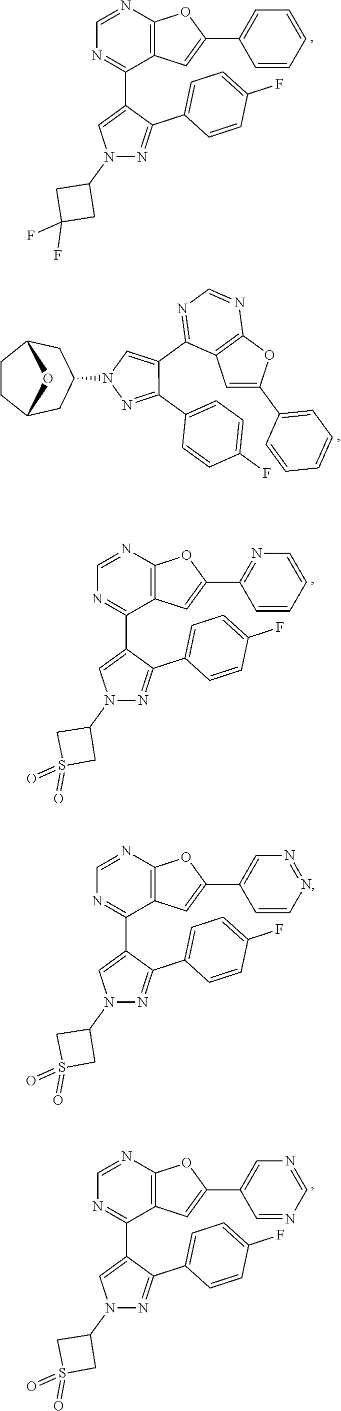 [SMILES: Fc1ccc(-c2nn(C3CC(F)(F)C3)cc2-c2ncnc3oc(-c4ccccc4)cc23)cc1 ; Fc1ccc(-c2nn([C@H]3C[C@H]4CC[C@@H](C3)O4)cc2-c2ncnc3oc(-c4ccccc4)cc23)cc1 ; O=S1(=O)CC(n2cc(-c3ncnc4oc(-c5ccccn5)cc34)c(-c3ccc(F)cc3)n2)C1 ; O=S1(=O)CC(n2cc(-c3ncnc4oc(-c5ccnnc5)cc34)c(-c3ccc(F)cc3)n2)C1 ; O=S1(=O)CC(n2cc(-c3ncnc4oc(-c5cncnc5)cc34)c(-c3ccc(F)cc3)n2)C1] 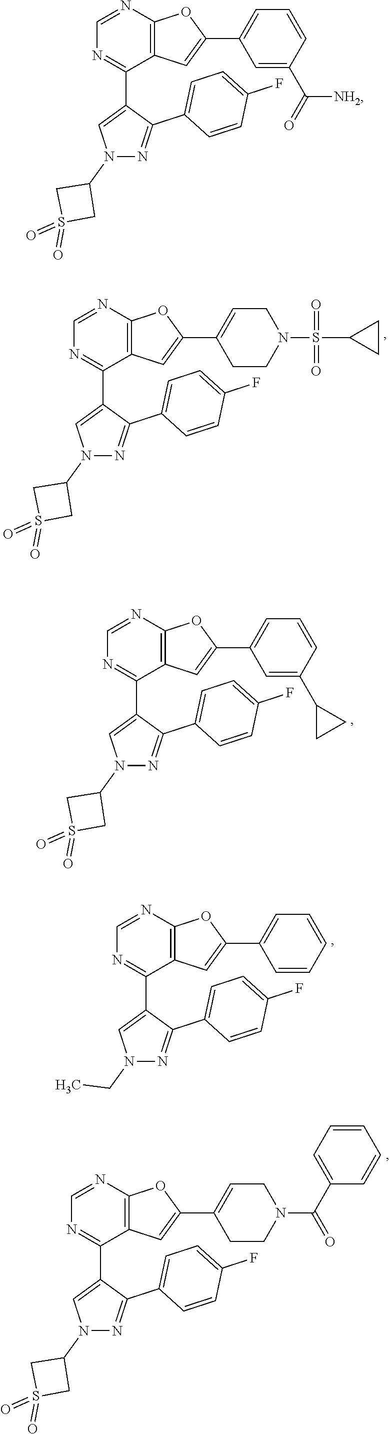 [SMILES: NC(=O)c1cccc(-c2cc3c(-c4cn(C5CS(=O)(=O)C5)nc4-c4ccc(F)cc4)ncnc3o2)c1 ; O=S1(=O)CC(n2cc(-c3ncnc4oc(C5=CCN(S(=O)(=O)C6CC6)CC5)cc34)c(-c3ccc(F)cc3)n2)C1 ; O=S1(=O)CC(n2cc(-c3ncnc4oc(-c5cccc(C6CC6)c5)cc34)c(-c3ccc(F)cc3)n2)C1 ; CCn1cc(-c2ncnc3oc(-c4ccccc4)cc23)c(-c2ccc(F)cc2)n1 ; O=C(c1ccccc1)N1CC=C(c2cc3c(-c4cn(C5CS(=O)(=O)C5)nc4-c4ccc(F)cc4)ncnc3o2)CC1] 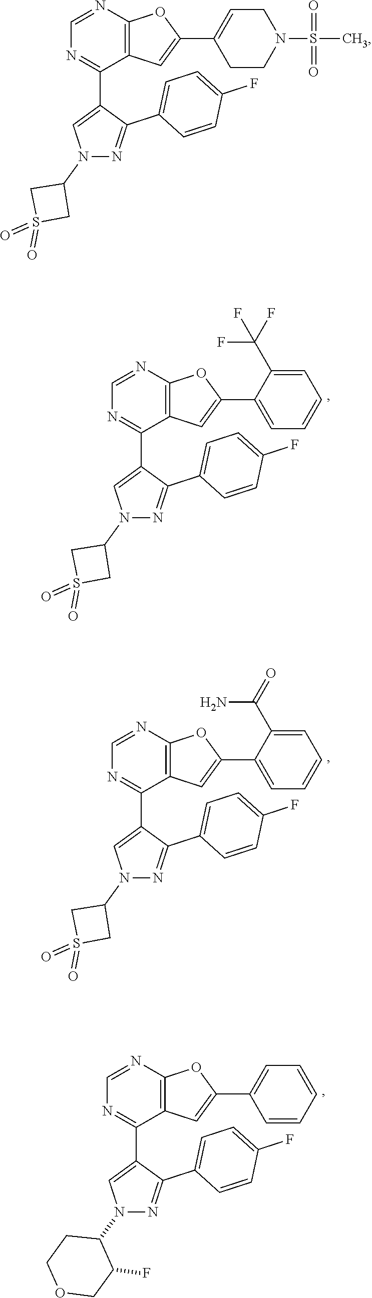 [SMILES: CS(=O)(=O)N1CC=C(c2cc3c(-c4cn(C5CS(=O)(=O)C5)nc4-c4ccc(F)cc4)ncnc3o2)CC1 ; O=S1(=O)CC(n2cc(-c3ncnc4oc(-c5ccccc5C(F)(F)F)cc34)c(-c3ccc(F)cc3)n2)C1 ; NC(=O)c1ccccc1-c1cc2c(-c3cn(C4CS(=O)(=O)C4)nc3-c3ccc(F)cc3)ncnc2o1 ; Fc1ccc(-c2nn([C@H]3CCOC[C@H]3F)cc2-c2ncnc3oc(-c4ccccc4)cc23)cc1] 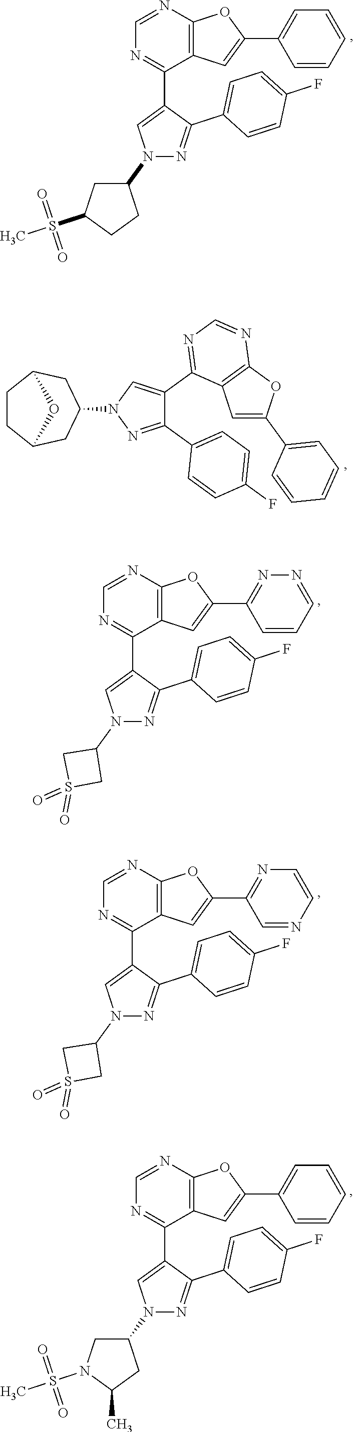 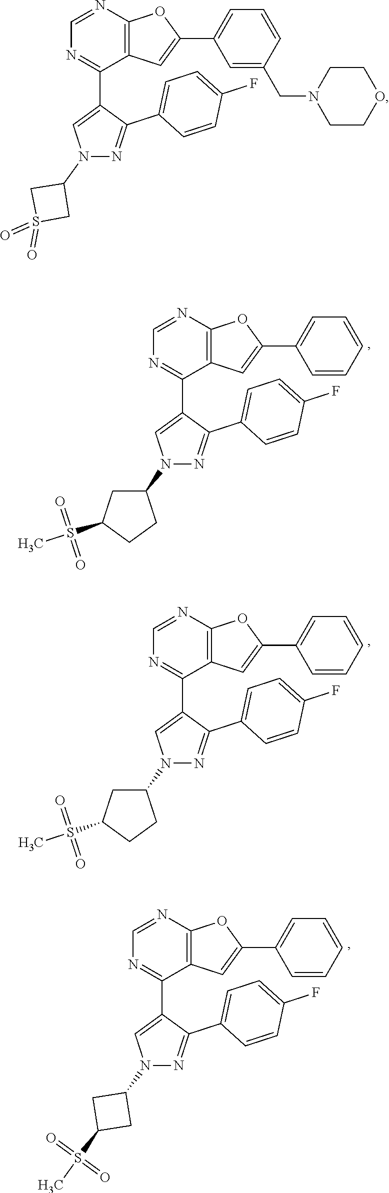 [SMILES: O=S1(=O)CC(n2cc(-c3ncnc4oc(-c5cccc(CN6CCOCC6)c5)cc34)c(-c3ccc(F)cc3)n2)C1 ; CS(=O)(=O)[C@@H]1CC[C@H](n2cc(-c3ncnc4oc(-c5ccccc5)cc34)c(-c3ccc(F)cc3)n2)C1 ; CS(=O)(=O)[C@H]1CC[C@@H](n2cc(-c3ncnc4oc(-c5ccccc5)cc34)c(-c3ccc(F)cc3)n2)C1 ; CS(=O)(=O)[C@H]1C[C@H](n2cc(-c3ncnc4oc(-c5ccccc5)cc34)c(-c3ccc(F)cc3)n2)C1] 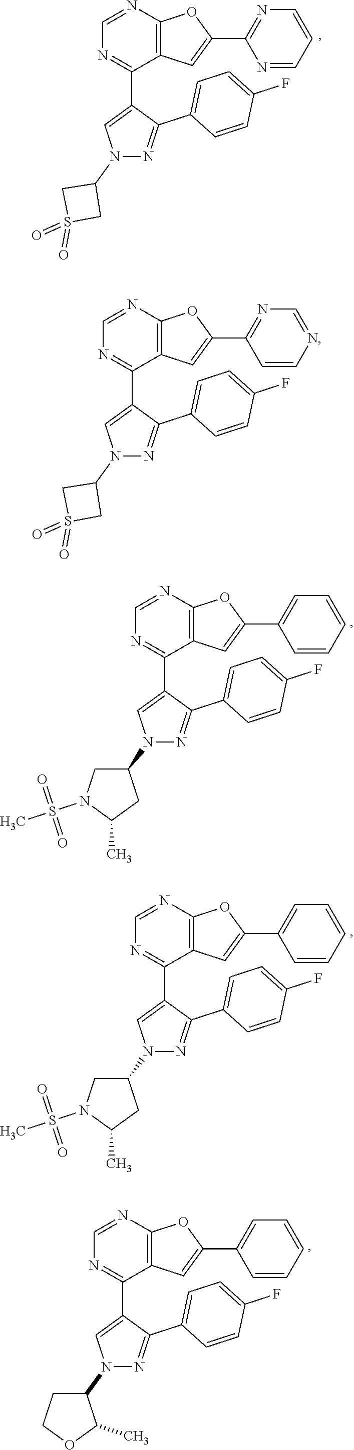 [SMILES: O=S1(=O)CC(n2cc(-c3ncnc4oc(-c5ncccn5)cc34)c(-c3ccc(F)cc3)n2)C1 ; O=S1(=O)CC(n2cc(-c3ncnc4oc(-c5ccncn5)cc34)c(-c3ccc(F)cc3)n2)C1 ; C[C@H]1C[C@H](n2cc(-c3ncnc4oc(-c5ccccc5)cc34)c(-c3ccc(F)cc3)n2)CN1S(C)(=O)=O ; C[C@H]1C[C@@H](n2cc(-c3ncnc4oc(-c5ccccc5)cc34)c(-c3ccc(F)cc3)n2)CN1S(C)(=O)=O ; C[C@@H]1OCC[C@H]1n1cc(-c2ncnc3oc(-c4ccccc4)cc23)c(-c2ccc(F)cc2)n1] 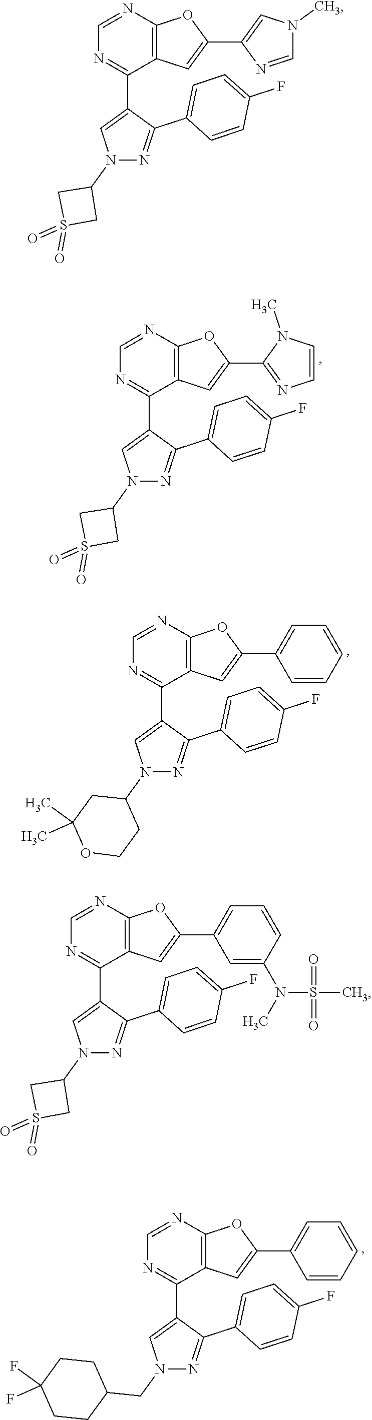 [SMILES: Cn1cnc(-c2cc3c(-c4cn(C5CS(=O)(=O)C5)nc4-c4ccc(F)cc4)ncnc3o2)c1 ; Cn1ccnc1-c1cc2c(-c3cn(C4CS(=O)(=O)C4)nc3-c3ccc(F)cc3)ncnc2o1 ; CC1(C)CC(n2cc(-c3ncnc4oc(-c5ccccc5)cc34)c(-c3ccc(F)cc3)n2)CCO1 ; CN(c1cccc(-c2cc3c(-c4cn(C5CS(=O)(=O)C5)nc4-c4ccc(F)cc4)ncnc3o2)c1)S(C)(=O)=O ; Fc1ccc(-c2nn(CC3CCC(F)(F)CC3)cc2-c2ncnc3oc(-c4ccccc4)cc23)cc1] 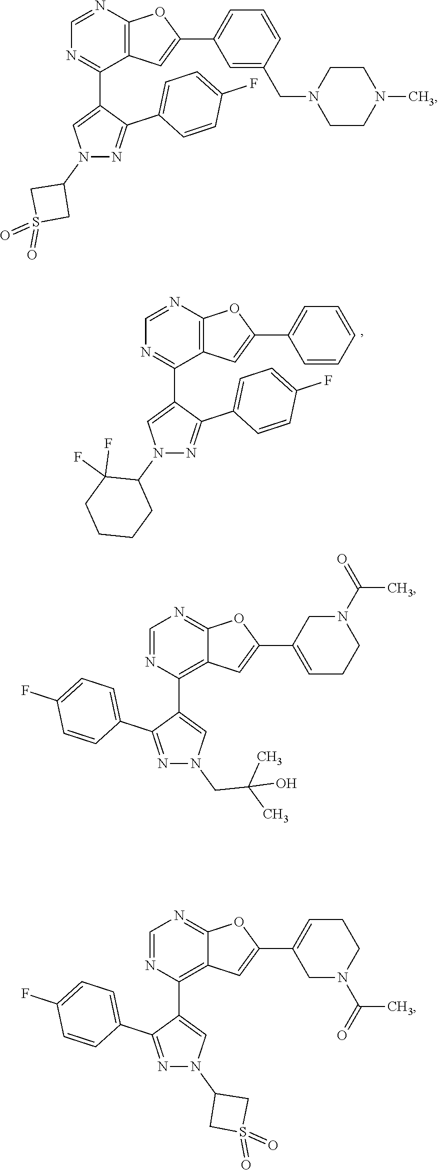 [SMILES: CN1CCN(Cc2cccc(-c3cc4c(-c5cn(C6CS(=O)(=O)C6)nc5-c5ccc(F)cc5)ncnc4o3)c2)CC1 ; Fc1ccc(-c2nn(C3CCCCC3(F)F)cc2-c2ncnc3oc(-c4ccccc4)cc23)cc1 ; CC(=O)N1CCC=C(c2cc3c(-c4cn(CC(C)(C)O)nc4-c4ccc(F)cc4)ncnc3o2)C1 ; CC(=O)N1CCC=C(c2cc3c(-c4cn(C5CS(=O)(=O)C5)nc4-c4ccc(F)cc4)ncnc3o2)C1] 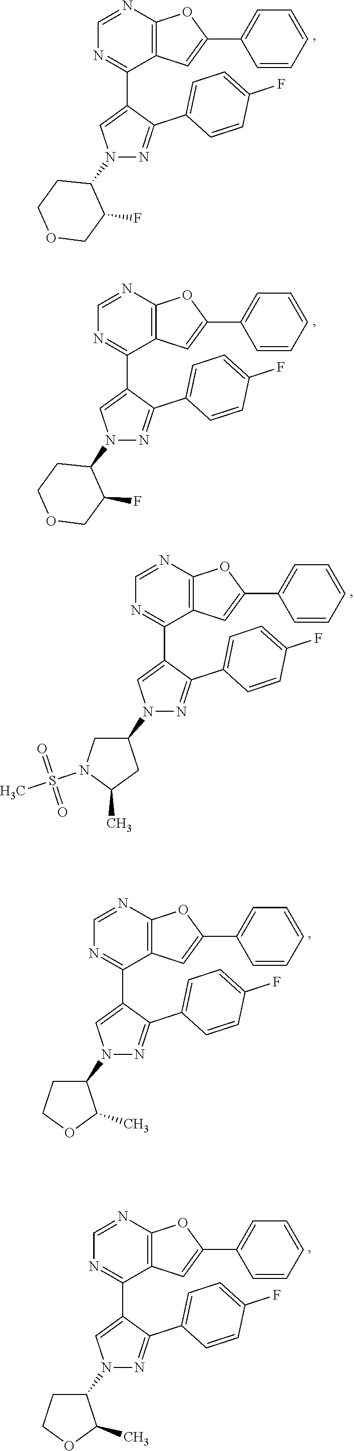 [SMILES: Fc1ccc(-c2nn([C@H]3CCOC[C@H]3F)cc2-c2ncnc3oc(-c4ccccc4)cc23)cc1 ; Fc1ccc(-c2nn([C@@H]3CCOC[C@@H]3F)cc2-c2ncnc3oc(-c4ccccc4)cc23)cc1 ; C[C@@H]1C[C@H](n2cc(-c3ncnc4oc(-c5ccccc5)cc34)c(-c3ccc(F)cc3)n2)CN1S(C)(=O)=O ; C[C@@H]1OCC[C@H]1n1cc(-c2ncnc3oc(-c4ccccc4)cc23)c(-c2ccc(F)cc2)n1 ; C[C@H]1OCC[C@@H]1n1cc(-c2ncnc3oc(-c4ccccc4)cc23)c(-c2ccc(F)cc2)n1] 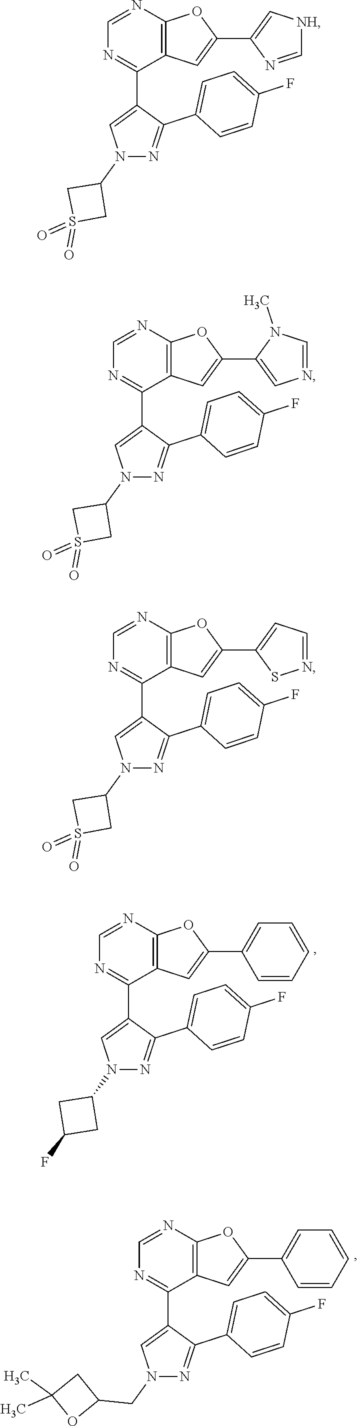 [SMILES: O=S1(=O)CC(n2cc(-c3ncnc4oc(-c5c[nH]cn5)cc34)c(-c3ccc(F)cc3)n2)C1 ; Cn1cncc1-c1cc2c(-c3cn(C4CS(=O)(=O)C4)nc3-c3ccc(F)cc3)ncnc2o1 ; O=S1(=O)CC(n2cc(-c3ncnc4oc(-c5ccns5)cc34)c(-c3ccc(F)cc3)n2)C1 ; Fc1ccc(-c2nn([C@H]3C[C@H](F)C3)cc2-c2ncnc3oc(-c4ccccc4)cc23)cc1 ; CC1(C)CC(Cn2cc(-c3ncnc4oc(-c5ccccc5)cc34)c(-c3ccc(F)cc3)n2)O1] 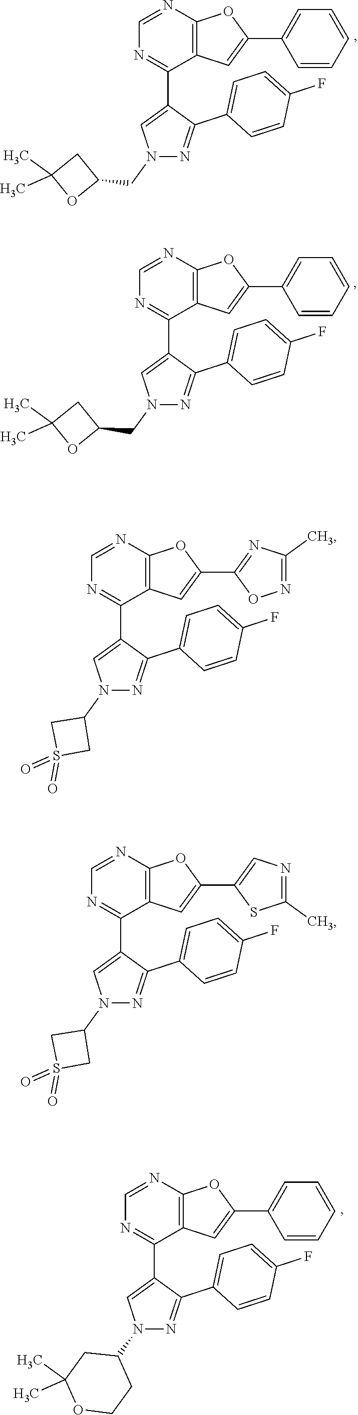 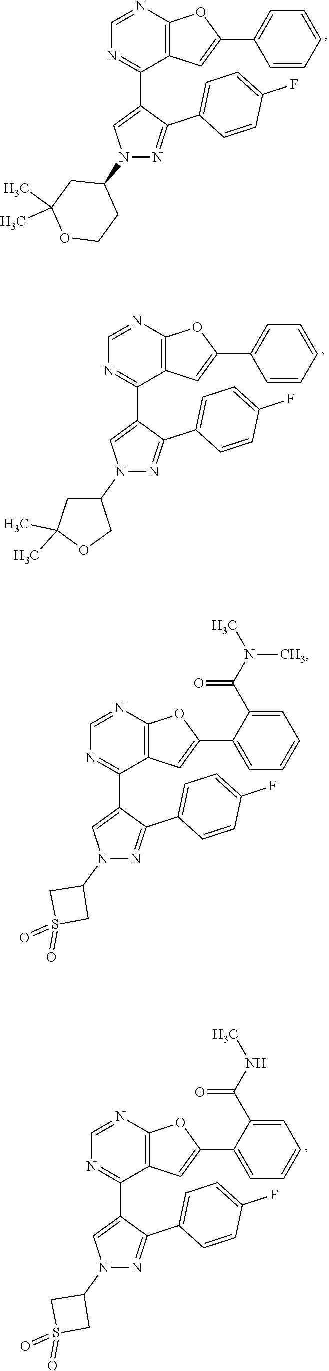 [SMILES: CC1(C)C[C@@H](n2cc(-c3ncnc4oc(-c5ccccc5)cc34)c(-c3ccc(F)cc3)n2)CCO1 ; CC1(C)CC(n2cc(-c3ncnc4oc(-c5ccccc5)cc34)c(-c3ccc(F)cc3)n2)CO1 ; CN(C)C(=O)c1ccccc1-c1cc2c(-c3cn(C4CS(=O)(=O)C4)nc3-c3ccc(F)cc3)ncnc2o1 ; CNC(=O)c1ccccc1-c1cc2c(-c3cn(C4CS(=O)(=O)C4)nc3-c3ccc(F)cc3)ncnc2o1] 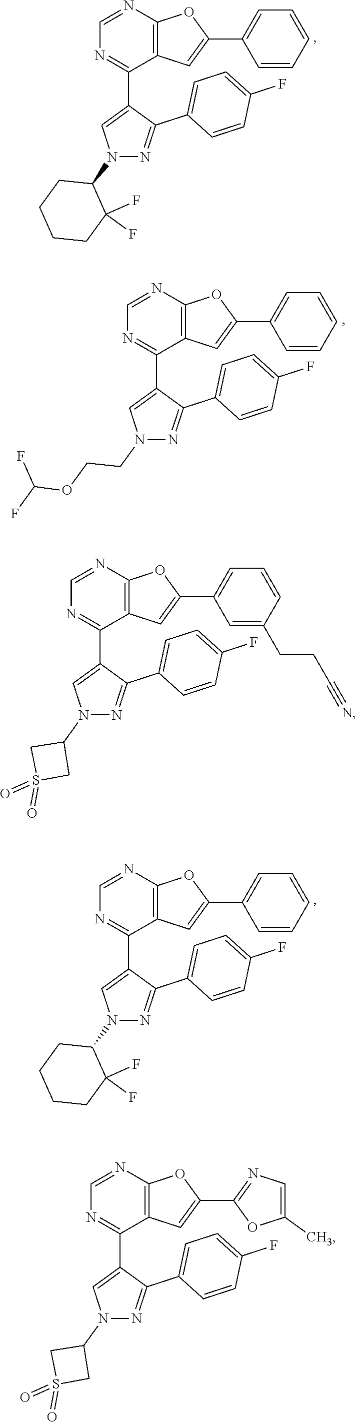 [SMILES: Fc1ccc(-c2nn([C@@H]3CCCCC3(F)F)cc2-c2ncnc3oc(-c4ccccc4)cc23)cc1 ; Fc1ccc(-c2nn(CCOC(F)F)cc2-c2ncnc3oc(-c4ccccc4)cc23)cc1 ; N#CCCc1cccc(-c2cc3c(-c4cn(C5CS(=O)(=O)C5)nc4-c4ccc(F)cc4)ncnc3o2)c1 ; Fc1ccc(-c2nn([C@H]3CCCCC3(F)F)cc2-c2ncnc3oc(-c4ccccc4)cc23)cc1 ; Cc1cnc(-c2cc3c(-c4cn(C5CS(=O)(=O)C5)nc4-c4ccc(F)cc4)ncnc3o2)o1] 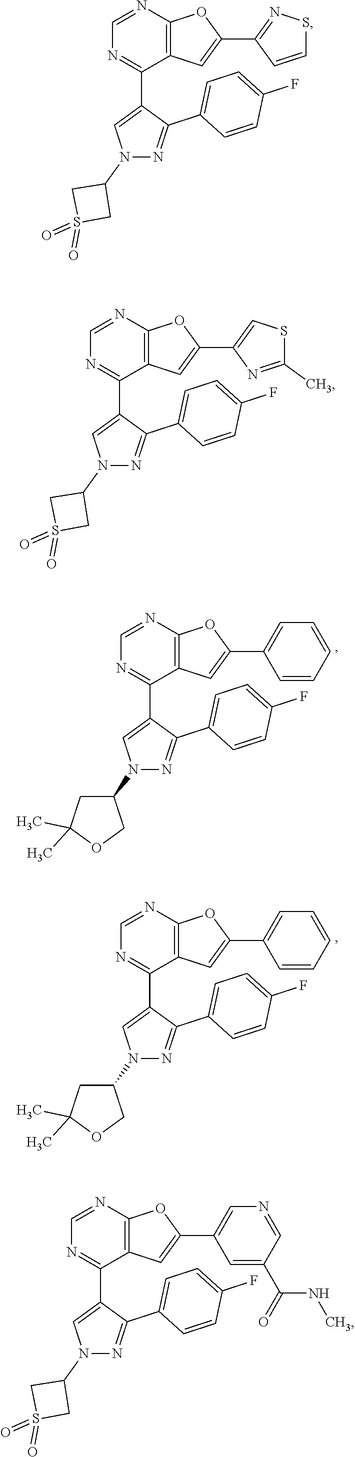 [SMILES: O=S1(=O)CC(n2cc(-c3ncnc4oc(-c5ccsn5)cc34)c(-c3ccc(F)cc3)n2)C1 ; Cc1nc(-c2cc3c(-c4cn(C5CS(=O)(=O)C5)nc4-c4ccc(F)cc4)ncnc3o2)cs1 ; CC1(C)C[C@@H](n2cc(-c3ncnc4oc(-c5ccccc5)cc34)c(-c3ccc(F)cc3)n2)CO1 ; CC1(C)C[C@H](n2cc(-c3ncnc4oc(-c5ccccc5)cc34)c(-c3ccc(F)cc3)n2)CO1 ; CNC(=O)c1cncc(-c2cc3c(-c4cn(C5CS(=O)(=O)C5)nc4-c4ccc(F)cc4)ncnc3o2)c1] 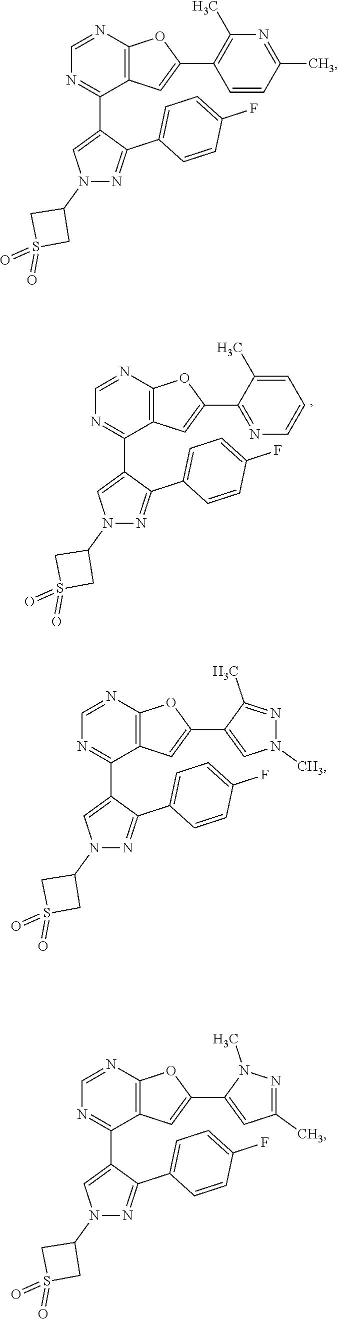 [SMILES: Cc1ccc(-c2cc3c(-c4cn(C5CS(=O)(=O)C5)nc4-c4ccc(F)cc4)ncnc3o2)c(C)n1 ; Cc1cccnc1-c1cc2c(-c3cn(C4CS(=O)(=O)C4)nc3-c3ccc(F)cc3)ncnc2o1 ; Cc1nn(C)cc1-c1cc2c(-c3cn(C4CS(=O)(=O)C4)nc3-c3ccc(F)cc3)ncnc2o1 ; Cc1cc(-c2cc3c(-c4cn(C5CS(=O)(=O)C5)nc4-c4ccc(F)cc4)ncnc3o2)n(C)n1] 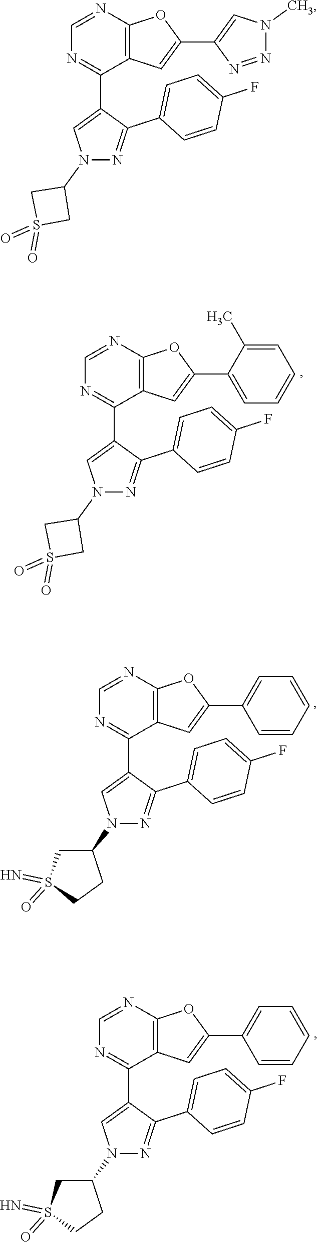 [SMILES: Cn1cc(-c2cc3c(-c4cn(C5CS(=O)(=O)C5)nc4-c4ccc(F)cc4)ncnc3o2)nn1 ; Cc1ccccc1-c1cc2c(-c3cn(C4CS(=O)(=O)C4)nc3-c3ccc(F)cc3)ncnc2o1 ; N=[S@]1(=O)CC[C@H](n2cc(-c3ncnc4oc(-c5ccccc5)cc34)c(-c3ccc(F)cc3)n2)C1 ; N=[S@@]1(=O)CC[C@@H](n2cc(-c3ncnc4oc(-c5ccccc5)cc34)c(-c3ccc(F)cc3)n2)C1] 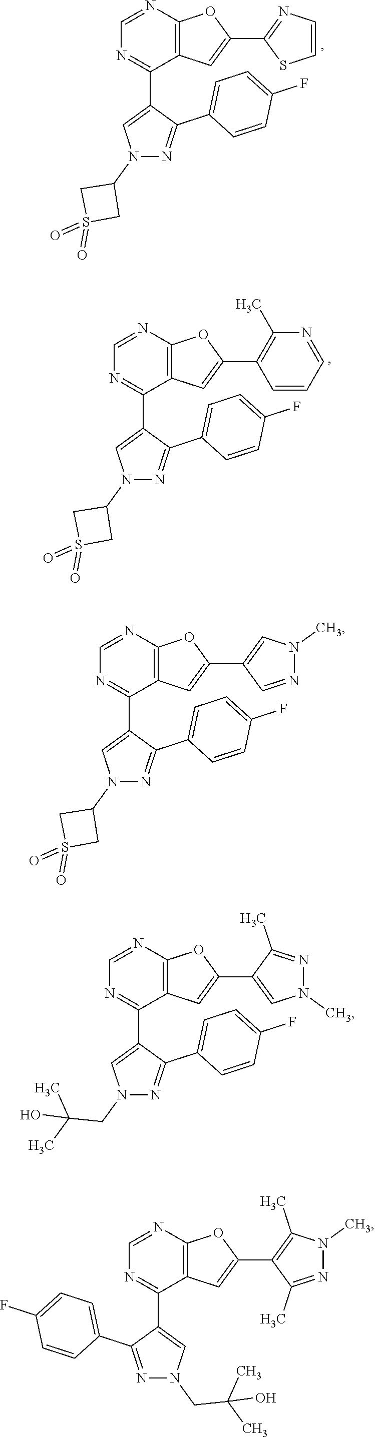 [SMILES: O=S1(=O)CC(n2cc(-c3ncnc4oc(-c5nccs5)cc34)c(-c3ccc(F)cc3)n2)C1 ; Cc1ncccc1-c1cc2c(-c3cn(C4CS(=O)(=O)C4)nc3-c3ccc(F)cc3)ncnc2o1 ; Cn1cc(-c2cc3c(-c4cn(C5CS(=O)(=O)C5)nc4-c4ccc(F)cc4)ncnc3o2)cn1 ; Cc1nn(C)cc1-c1cc2c(-c3cn(CC(C)(C)O)nc3-c3ccc(F)cc3)ncnc2o1 ; Cc1nn(C)c(C)c1-c1cc2c(-c3cn(CC(C)(C)O)nc3-c3ccc(F)cc3)ncnc2o1] 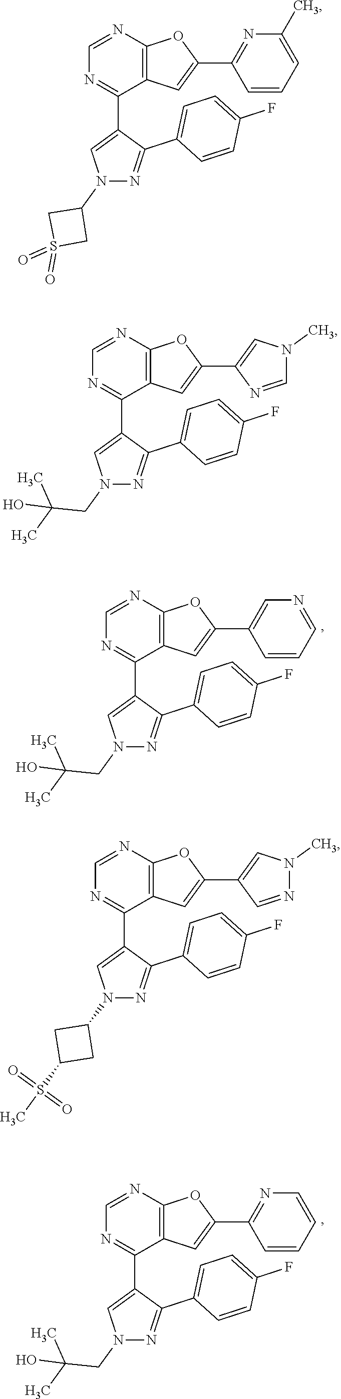 [SMILES: Cc1cccc(-c2cc3c(-c4cn(C5CS(=O)(=O)C5)nc4-c4ccc(F)cc4)ncnc3o2)n1 ; Cn1cnc(-c2cc3c(-c4cn(CC(C)(C)O)nc4-c4ccc(F)cc4)ncnc3o2)c1 ; CC(C)(O)Cn1cc(-c2ncnc3oc(-c4cccnc4)cc23)c(-c2ccc(F)cc2)n1 ; Cn1cc(-c2cc3c(-c4cn([C@H]5C[C@@H](S(C)(=O)=O)C5)nc4-c4ccc(F)cc4)ncnc3o2)cn1 ; CC(C)(O)Cn1cc(-c2ncnc3oc(-c4ccccn4)cc23)c(-c2ccc(F)cc2)n1] 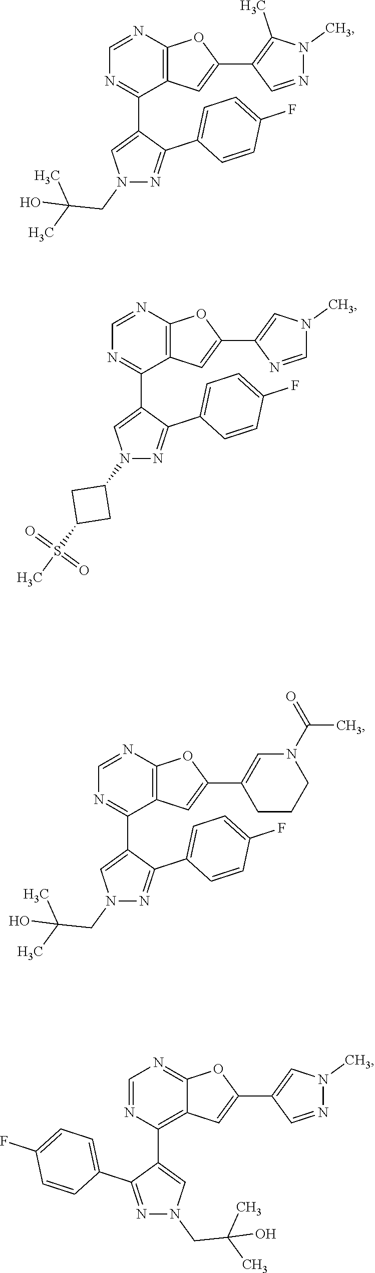 [SMILES: Cc1c(-c2cc3c(-c4cn(CC(C)(C)O)nc4-c4ccc(F)cc4)ncnc3o2)cnn1C ; Cn1cnc(-c2cc3c(-c4cn([C@H]5C[C@@H](S(C)(=O)=O)C5)nc4-c4ccc(F)cc4)ncnc3o2)c1 ; CC(=O)N1C=C(c2cc3c(-c4cn(CC(C)(C)O)nc4-c4ccc(F)cc4)ncnc3o2)CCC1 ; Cn1cc(-c2cc3c(-c4cn(CC(C)(C)O)nc4-c4ccc(F)cc4)ncnc3o2)cn1] 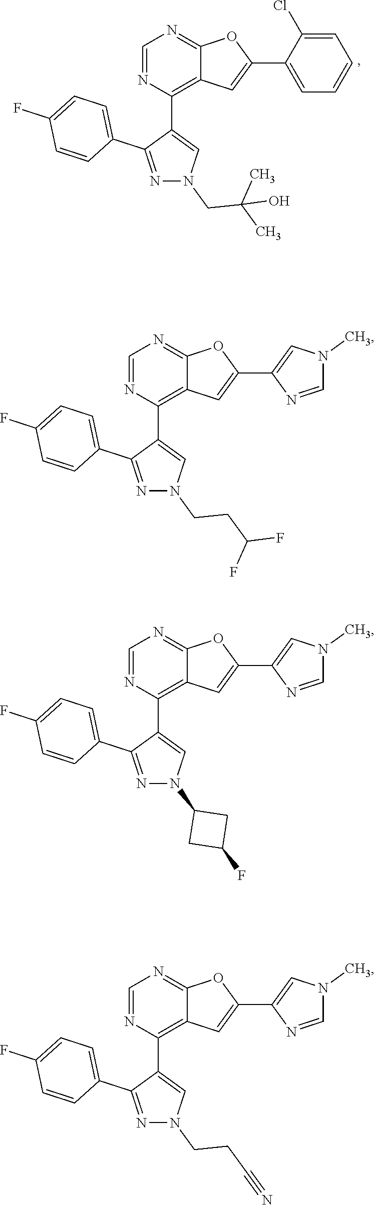 [SMILES: CC(C)(O)Cn1cc(-c2ncnc3oc(-c4ccccc4Cl)cc23)c(-c2ccc(F)cc2)n1 ; Cn1cnc(-c2cc3c(-c4cn(CCC(F)F)nc4-c4ccc(F)cc4)ncnc3o2)c1 ; Cn1cnc(-c2cc3c(-c4cn([C@H]5C[C@@H](F)C5)nc4-c4ccc(F)cc4)ncnc3o2)c1 ; Cn1cnc(-c2cc3c(-c4cn(CCC#N)nc4-c4ccc(F)cc4)ncnc3o2)c1] 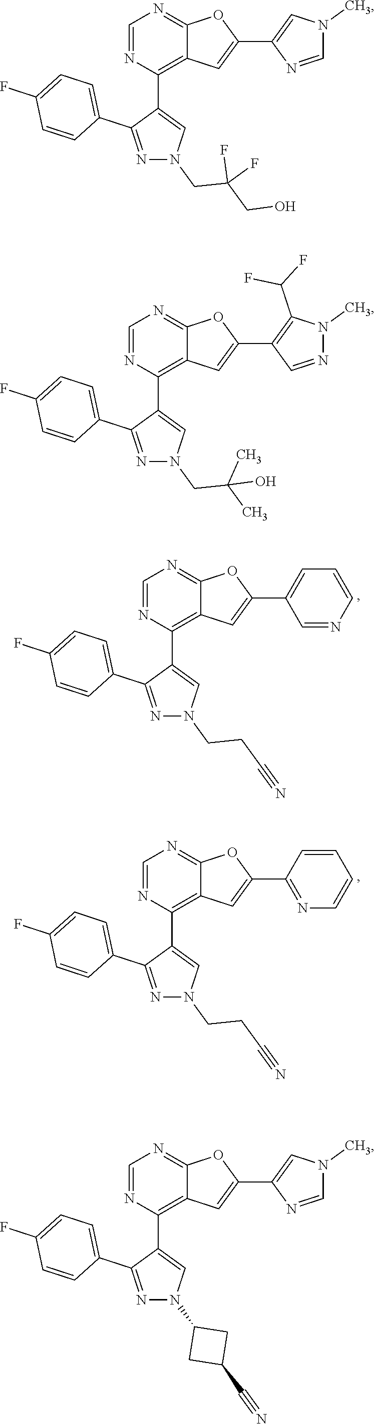 [SMILES: Cn1cnc(-c2cc3c(-c4cn(CC(F)(F)CO)nc4-c4ccc(F)cc4)ncnc3o2)c1 ; Cn1ncc(-c2cc3c(-c4cn(CC(C)(C)O)nc4-c4ccc(F)cc4)ncnc3o2)c1C(F)F ; N#CCCn1cc(-c2ncnc3oc(-c4cccnc4)cc23)c(-c2ccc(F)cc2)n1 ; N#CCCn1cc(-c2ncnc3oc(-c4ccccn4)cc23)c(-c2ccc(F)cc2)n1 ; Cn1cnc(-c2cc3c(-c4cn([C@H]5C[C@H](C#N)C5)nc4-c4ccc(F)cc4)ncnc3o2)c1] 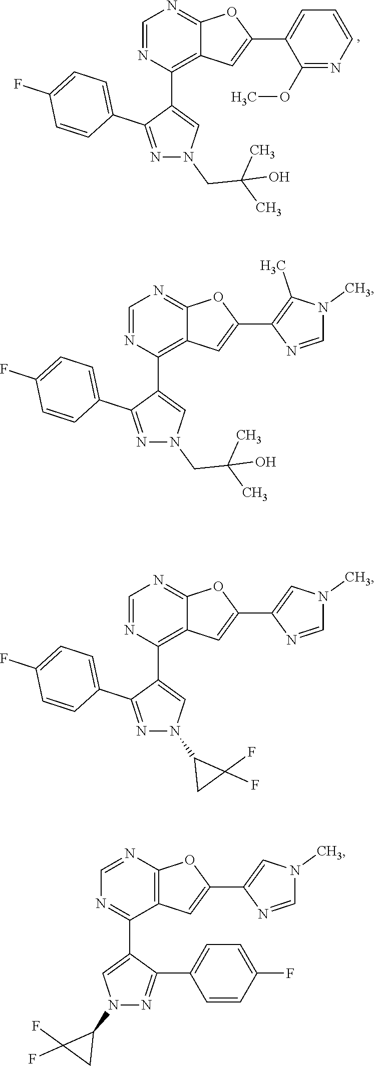 [SMILES: COc1ncccc1-c1cc2c(-c3cn(CC(C)(C)O)nc3-c3ccc(F)cc3)ncnc2o1 ; Cc1c(-c2cc3c(-c4cn(CC(C)(C)O)nc4-c4ccc(F)cc4)ncnc3o2)ncn1C ; Cn1cnc(-c2cc3c(-c4cn([C@H]5CC5(F)F)nc4-c4ccc(F)cc4)ncnc3o2)c1 ; Cn1cnc(-c2cc3c(-c4cn([C@H]5CC5(F)F)nc4-c4ccc(F)cc4)ncnc3o2)c1] 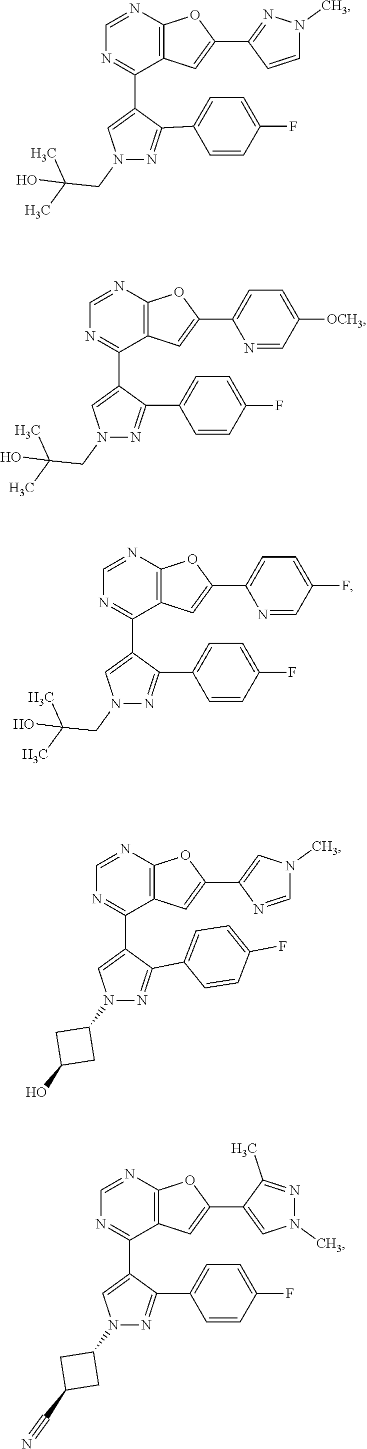 [SMILES: Cn1ccc(-c2cc3c(-c4cn(CC(C)(C)O)nc4-c4ccc(F)cc4)ncnc3o2)n1 ; COc1ccc(-c2cc3c(-c4cn(CC(C)(C)O)nc4-c4ccc(F)cc4)ncnc3o2)nc1 ; CC(C)(O)Cn1cc(-c2ncnc3oc(-c4ccc(F)cn4)cc23)c(-c2ccc(F)cc2)n1 ; Cn1cnc(-c2cc3c(-c4cn([C@H]5C[C@H](O)C5)nc4-c4ccc(F)cc4)ncnc3o2)c1 ; Cc1nn(C)cc1-c1cc2c(-c3cn([C@H]4C[C@H](C#N)C4)nc3-c3ccc(F)cc3)ncnc2o1] 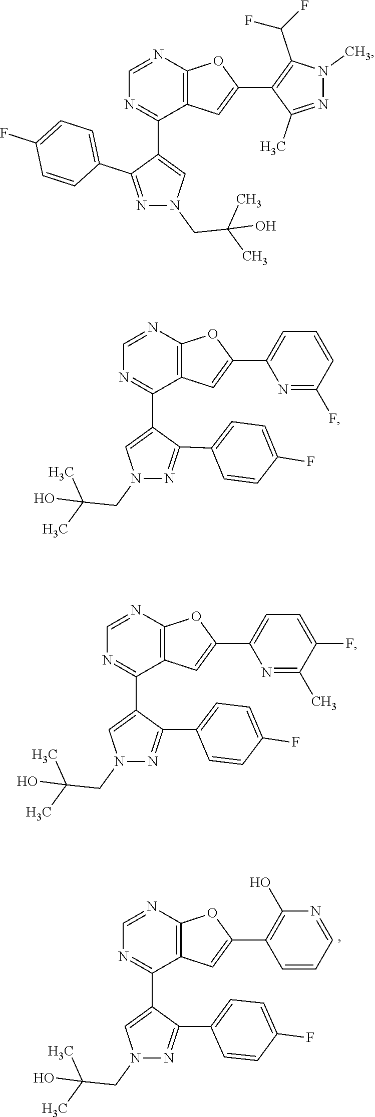 [SMILES: Cc1nn(C)c(C(F)F)c1-c1cc2c(-c3cn(CC(C)(C)O)nc3-c3ccc(F)cc3)ncnc2o1 ; CC(C)(O)Cn1cc(-c2ncnc3oc(-c4cccc(F)n4)cc23)c(-c2ccc(F)cc2)n1 ; Cc1nc(-c2cc3c(-c4cn(CC(C)(C)O)nc4-c4ccc(F)cc4)ncnc3o2)ccc1F ; CC(C)(O)Cn1cc(-c2ncnc3oc(-c4cccnc4O)cc23)c(-c2ccc(F)cc2)n1] 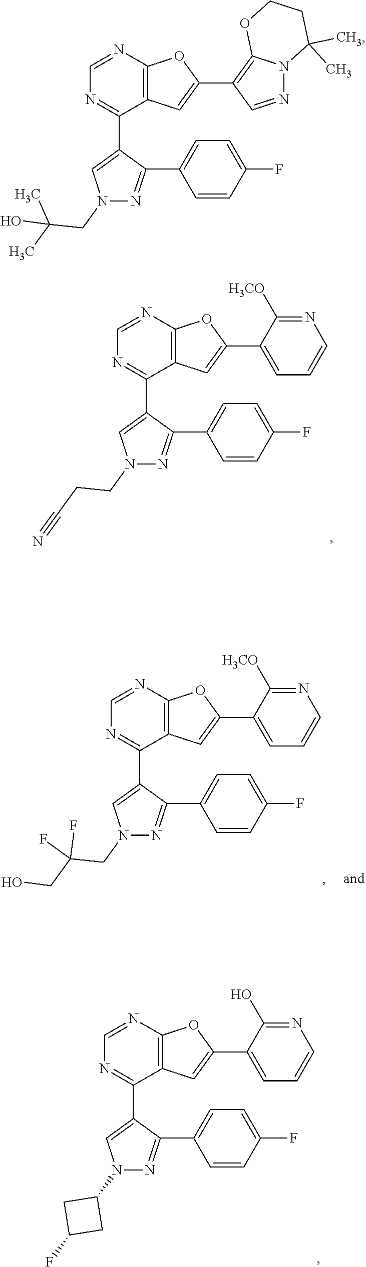 [SMILES: CC(C)(O)Cn1cc(-c2ncnc3oc(-c4cnn5c4OCCC5(C)C)cc23)c(-c2ccc(F)cc2)n1 ; COc1ncccc1-c1cc2c(-c3cn(CCC#N)nc3-c3ccc(F)cc3)ncnc2o1 ; COc1ncccc1-c1cc2c(-c3cn(CC(F)(F)CO)nc3-c3ccc(F)cc3)ncnc2o1 ; Oc1ncccc1-c1cc2c(-c3cn([C@H]4C[C@@H](F)C4)nc3-c3ccc(F)cc3)ncnc2o1]
or a pharmaceutically acceptable salt thereof.

12. A method of treating cancer in a subject, wherein the cancer has been determined to comprise a C797S mutation in the epidermal growth factor receptor (EGFR) protein, comprising administering to the subject a compound according to any one of claims 1 to 11, or a pharmaceutically acceptable salt thereof.

13. A method of treating cancer in a subject, wherein the cancer has been determined to comprise L858R and C797S mutations in the epidermal growth factor receptor (EGFR) protein, comprising administering to the subject a compound according to any one of claims 1 to 11, or a pharmaceutically acceptable salt thereof.

14. A method of treating metastatic cancer in a subject, wherein the metastatic cancer has been determined to comprise exon 19 deletion and C797S mutations in the epidermal growth factor receptor (EGFR) protein, comprising administering to the subject a compound according to any one of claims 1 to 11, or a pharmaceutically acceptable salt thereof.

15. The method of any one of claim 12 to 14, wherein the cancer is selected from the group consisting of breast cancer, colon cancer, lung cancer, non-small cell lung cancer, gastric cancer, prostate cancer, and colorectal cancer.

16. The method of any one of claims 12 to 15, wherein the cancer comprises one or more central nervous system (CNS) metastases.

17. Use of a composition comprising a compound according to any one of claims 1 to 11, or a pharmaceutically acceptable salt thereof, for manufacture of a medicament for treatment of a subject having cancer, wherein the cancer comprises a C797S mutation in the epidermal growth factor receptor (EGFR) protein.

18. Use of a composition comprising a compound according to any one of claims 1 to 11, or a pharmaceutically acceptable salt thereof, for manufacture of a medicament for the treatment of cancer in a subject, wherein the cancer comprises (a) one or more central nervous system (CNS) metastases, and (b) a C797S mutation in the epidermal growth factor receptor (EGFR) protein.
